(19) United States
(12) Patent Application Publication
(10) Pub. No.: US 2020/0247812 A1
(43) Pub. Date: Aug. 6, 2020

(54) HETEROCYCLIC COMPOUNDS AND METHODS OF USE

(71) Applicant: Integral Biosciences Private Limited, Noida (IN)

(72) Inventors: Sarvajit Chakravarty, Edmond, OK (US); Dhananjay Pendharkar, Greater Noida (IN); Brahmam Pujala, Greater Noida (IN); Anil Kumar Agarwal, Bangalore (IN); Varun Kumar, Pandav Nagar (IN); Satish Kumar Arya, Noida (IN)

(21) Appl. No.: 16/635,029

(22) PCT Filed: Jul. 9, 2019
(86) PCT No.: PCT/IB2019/055840

§ 371 Date: Jan. 29, 2020

(30) Foreign Application Priority Data
Jul. 10, 2018 (IN) 201811025792

(51) Int. Cl. C07D 487/04; C07D 401/12; C07D 471/04; C07D 519/00; C07D 401/14; C07D 413/14; C07D 417/14; C07D 409/14; C07D 407/14; C07D 498/04; C07D 513/04; A61P 35/00

(52) U.S. Cl. CPC C07D 487/04; C07D 401/12; C07D 471/04; C07D 519/00; C07D 401/14; A61P 35/00; C07D 417/14; C07D 409/14; C07D 407/14; C07D 498/04; C07D 513/04; C07D 413/14

(57) ABSTRACT

The present invention discloses compounds useful in treatment of conditions associated with excessive activity of transforming growth factor beta (TGF-β), particularly type I or activin-like kinase 5 (ALK 5). Specifically, the present invention discloses compound of formula (I)

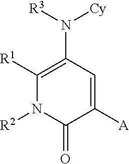

which exhibit inhibitory activity against ALK 5. Method of treating conditions associated with excessive activity (ALK 5) with such compound is disclosed. Uses thereof, pharmaceutical composition, and kits are also disclosed.

### CROSS REFERENCE TO RELATED APPLICATION

This application claims the priority benefit of INDIAN Provisional Patent Application No. 201811025792, filed Jul. 10, 2018, the disclosures of which are incorporated herein by reference in their entireties.

### FIELD OF THE INVENTION

The present invention relates generally to compounds useful in treatment of conditions associated with excessive activity of transforming growth factor beta (TGF-β), particularly type 1 or activin-like kinase 5 (ALK 5). The invention also provides method of using said compounds and pharmaceutically acceptable compositions comprising compounds of the present invention.

### BACKGROUND OF THE INVENTION

Transforming growth factors, including TGF-β, play a key role in controlling cellular functions such as proliferation, differentiation, migration, extracellular matrix production, apoptosis, adhesion, and development. Dysregulated TGF-β signaling has been identified as a key factor in a number of pathological disorders.

TGF-β and other cytokines signal through a complex of two structurally and functionally distinct transmembrane receptor serine/threonine kinases, known as type 1 and type 2 TGF-β receptors, resulting in activation of TGF-β mediated pathways. The type 1 TGF-β receptor is also known as activin-like kinase 5 (ALK 5). Inhibition of ALK 5 antagonizes TGF-β mediated pathways and provides diverse biological effects in clinical applications, including the treatment of cancer, fibrosis, cardiovascular disorders, wound healing, and many others.

TGF-β superfamily of conserved cytokines, growth factors, and morphogens consists of isoforms TGF-β1 (ALK 5), TGF-β2, and TGF-β3. These proteins are pleiotropic modulators that regulates various biological processes including cell growth and differentiation, stimulating cell proliferation, inducing apoptosis, hematopoiesis, embryonic and bone development, immune and inflammatory responses and extracellular matrix formation (Roberts and Sporn Handbook of Experimental Pharmacology (1990) 95:419-58; Massague, et aI., Ann. Rev. Cell. Biol. (1990) 6:597-646). Among the various isoforms, TGF-β1 inhibits the growth of epithelial cells and stimulates the proliferation of mesenchymal cells. Activin, inhibin, bone morphogenic protein, and Mullerian inhibiting substance (MIS) are other members of this superfamily. TGF-β superfamily members are responsible for initiation of intracellular signaling pathways that ultimately leads to gene expressions that regulate cell cycle. The TGF-β superfamily also control proliferative responses, cell adhesion, intercellular communication, cellular migration and relate with extracellular matrix proteins that mediate outside-in cell signaling.

As TGF-β regulates many biological processes, dysregulation in its activity can be harmful. For example, TGF-β stimulates the proliferation of mesenchymal cells under normal conditions, however when dysregulated, TGF-β can be an autocrine growth factor for many tumors. Similarly, overexpression of TGF-β can lead to an excessive accumulation of extracellular matrix resulting in fibroproliferative diseases. Hence, there is need to develop compounds that modulate the TGF-β signaling pathway.

Inhibition of TGF-β intracellular signaling pathway can be primarily recognized for treatment of fibroproliferative diseases and cancer. Fibroproliferative diseases mainly involve kidney disorders that are associated with dysregulation of TGF-β activity and excessive fibrosis. The excessive fibrosis includes glomerulonephritis (GN) in form of mesangial proliferative GN, immune GN, and crescentic GN. Diabetic nephropathy, renal interstitial fibrosis and renal fibrosis in transplant patients receiving cyclosporine, and HIV-associated nephropathy are other renal conditions with enhanced TGF-β activity. Dermatomyositis, Eosinophilic fasciitis (Shulman's Syndrome), morphea, polymyositis, progressive systemic sclerosis, scleroderma, or those associated with the occurrence of Raynaud's syndrome are forms of collagen vascular disorders. In addition, excessive TGF-β activity can also contribute to lung fibroses that in turn include adult respiratory distress syndrome, chronic obstructive pulmonary disease (COPD), idiopathic pulmonary fibrosis and interstitial pulmonary fibrosis. Interstitial pulmonary fibrosis is usually associated with allergies, chemical contact, scleroderma and autoimmune disorders, such as systemic lupus erythematosus. Rheumatoid arthritis is another autoimmune disorder associated with fibroproliferative characteristics. Surgical eye procedures can also be linked with fibroproliferative conditions. These procedures include cataract extraction with intraocular lens implantation, post glaucoma drainage surgery and retinal reattachment surgery accompanying proliferative vitreoretinopathy.

Progression of various cancers is also mediated through members of the TGF-β superfamily (M. P. de Caestecker, E. Piek, and A. B. Roberts, J. National Cancer Inst., 92(17), 1388-1402 (2000) as these members are overexpressed significantly in many tumors. (Derynck, Trends Biochem. Sci., 1994, 19, 548-553). For example, TGF-β1 or ALK 5 is known to inhibit tumor formation by inhibition of the proliferation of non-transformed cells, however it promotes tumor growth once the tumor is formed. (N. Dumont and C. L. Arteaga, Breast Cancer Res., Vol. 2, 125-132 (2000)). Therefore, inhibition of the TGF-β pathway may be useful for the treatment of different types of cancer such as colorectal cancer, lung cancer and skin cancer. Particularly, TGF-β inhibition can be utilized for treatment of breast cancer, brain cancer, pancreatic cancer and glioma.

Many TGF-β inhibitors based on dihydropyrrolopyrazole, pyrazole, quinazoline, and imidazole cores were discovered by several companies including Eli Lilly, Scios, Biogen Idec, GlaxoSmithKline, Kyoto Pharmaceutical University and Kirin Brewery Company (Current Pharmaceutical Biotechnology, 2011, 12, 2190-2202). Among the reported small molecule TGF-β inhibitors, Eli Lilly's LY2157299, Medpacto's TEW 7197 and Merck's M7824 were progressed to clinical trials. Fresolimumab, developed by Cambridge Antibody Technology, is a human monoclonal antibody targeting all isoforms of TGF-β which is currently in phase 2 clinical trials.

Small molecule modulator of TGF-β are described in patents/patent applications WO2005/065691 (PCT/US2004/043503), U.S. Ser. No. 13/508,090, WO2011/146287A1, WO2004/065392, WO2009/050183, and WO2009/133070 (see for more examples). Antibodies to TGF-β have been described in U.S. Pat. Nos. 7,527,791; 7,927.593; 7,494,651; 7,369,111; 7,151,169; 6,492,497; 6,419,928; 6,090,383; 5,783,185; 5,772,998; 5,571,714; and 7,723,486.

There remains a continuing need for new therapies for the treatment of diseases and disorders related to the TGF-β dysregulation. Therefore, the present invention provides inhibitors of ALK 5.

### SUMMARY OF THE INVENTION

In one aspect, the present invention provides a compound of formula (I):

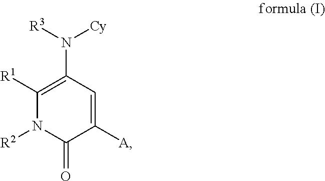

or a tautomer, salt, polymorph, solvate or stereoisomer thereof, wherein

Cy is

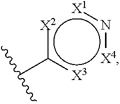

wherein

X¹ is N, CH or CR⁴;

X² is N, CH or CR⁵;

X³ is N, CH or CR⁶;

X⁴ is N, CH or CR⁷; wherein 0, 1 or 2 of X¹, X², X³ and X⁴ are N;

A is C₆ aryl, 5- to 6-membered heteroaryl, C₃-C₆ cycloalkyl or 5- to 6-membered heterocyclyl. wherein A is optionally substituted with 0 to 3 R⁹;

R¹ is independently hydrogen, halogen, —CN, C₁-C₆ alkyl, C₂-C₆ alkenyl, C₂-C₆ alkynyl, C₃-C₆ cycloalkyl, 3- to 6-membered heterocyclyl, 5- to 6-membered heteroaryl, C₁-C₃ alkoxy, C₁-C₃ haloalkoxy, C₁-C₃ haloalkyl, —OR¹¹, NR¹¹—N¹², C(O)NR¹²R¹³, —C(O)R¹¹, —OC(O)R¹¹, —C(O)OR¹¹, —OC(O)NR¹²R¹³, NR¹¹C(O)R¹², —NR¹¹C(O)OR¹², —NR¹¹C(O)NR¹²R¹³, —SR¹¹, —S(O)R¹¹, —S(O)₂R¹¹, NR¹¹S(O)R¹², —C(O)NR¹¹S(O)R¹², —NR¹¹S(O)₂R¹², —C(O)NR¹¹S(O)₂R¹², —S(O)NR¹²R¹³, —S(O)₂NR¹²R¹³, —(C₁-C₃ alkylene)CF₃, —(C₁-C₃ alkylene)OR¹¹, (C₁-C₃ alkylene) cycloalkyl, —(C₁-C₃ alkylene)NR¹²R¹³, —(C₁-C₃ alkylene)C(O)R¹¹, —(C₁-C₃ alkylene)C(O)OR¹¹, —(C₁-C₃ alkylene)C(O)NR¹²R¹³, —(C₁-C₃ alkylene) —C(O)OR¹¹, —(C₁-C₃ alkylene)OC(O)NR¹²R¹³, —(C₁-C₃ alkylene)S(O)R¹¹ or —(C₁-C₃ alkylene)S(O)₂R¹¹, wherein each R¹ is optionally substituted with R¹⁰;

R² is hydrogen, C₁-C₆ alkyl, C₂-C₆ alkenyl, C₂-C₆ alkynyl, C₃-C₆ cycloalkyl, 3- to 6-membered heterocyclyl, C₁-C₃ alkoxy, C₁-C₃ haloalkoxy, —C(O) R¹¹ or —(C₁-C₃ alkylene)OR¹¹;

or R¹ and R² are taken together with the atom to which they are attached to form a 5- to 7-membered heterocyclyl containing 1 to 3 heteroatom independently selected from N, O or S; each of which is optionally substituted by 0 to 3 R⁸;

R³ is hydrogen or C₁-C₆ alkyl;

R⁴, R⁵, R⁶ and R⁷ are independently hydrogen, halogen, oxo, —CN, C₁-C₆ alkyl, C₁-C₆ cycloalkyl, C₁-C₃ alkoxy, C₁-C₃ haloalkoxy, C₁-C₃ haloalkyl, 3- to 6-membered heterocyclyl, 5- to 6-membered heteroaryl, —NR¹¹R¹², S(O)₂NR¹²R¹³, —NR¹¹S(O)₂R¹², —C(O)NR¹²R¹³, —NR¹¹C(O)R¹² or —C(O)OR¹¹, each of which is optionally substituted by halogen, oxo, —CN, —OR¹⁵ or —NR¹⁵R¹⁶;

or R⁴ and R⁵ are taken together with the atom to which they are attached to form a 5- to 6-membered cycloalkyl or C₆ aryl; or 5- to 6-membered heterocyclyl or heteroaryl containing 1 to 3 heteroatom independently selected from N, O or S; each of which is optionally substituted by 0 to 3 R⁸;

each R⁸ is independently hydrogen, halogen, oxo, —CN, C₁-C₆ alkyl, C₁-C₆ cycloalkyl, C₁-C₃ alkoxy, C₁-C₃ haloalkoxy, C₁-C₃ haloalkyl, 3- to 6-membered heterocyclyl, —NR¹¹R¹², —C(O)NR¹²R¹³, —NR¹¹C(O)R¹², —C(O)OR¹¹, —OR¹¹, —(C₁-C₃ alkylene)NR¹¹R¹² or —(C₁-C₃ alkylene)3- to 6-membered heterocyclyl, each of which is optionally substituted by C₁-C₆ alkyl, halogen, oxo, —CN, —OR¹⁵ or —NR¹⁵R¹⁶;

each R⁹ is independently halogen, —CN, C₁-C₆ alkyl, C₃-C₆ cycloalkyl, 3- to 6-membered heterocyclyl, C₁-C₃haloalkoxy, C₁-C₃haloalkyl, —OR¹¹, —NR¹¹R¹², —C(O)NR¹²R¹³, —C(O)R¹¹, —SR¹¹, —S(O)R¹² or —S(O)₂R¹¹, each of which is optionally substituted by halogen, oxo, —CN, —OR¹⁵ or —NR¹⁵R¹⁶;

R¹⁰ is independently hydrogen, halogen, oxo, —CN, C₁-C₆ alkyl, C₃-C₆ cycloalkyl, 3- to 6-membered heterocyclyl, C₁-C₃haloalkoxy, C₁-C₃haloalkyl, —OR¹⁵, —NR¹⁵ R¹⁶, C(O)NR¹⁵R¹⁶, —C(O)R¹⁵, —SR¹⁵, —S(O)R¹⁵ or —S(O)₂R¹⁵;

R¹¹, R¹² and R¹³ are independently hydrogen, C₁-C₆ alkyl, C₂-C₆ alkenyl, C₂-C₆ alkynyl, C₃-C₆ cycloalkyl, 3- to 6-membered heterocyclyl, 5- to 6-membered heteroaryl or —(C₁-C₃ alkylene)OR¹⁵, each of which is optionally substituted with C₃-C₆ cycloalkyl, halogen, oxo, C₁-C₃ alkoxy, 3- to 6-membered heterocyclyl, —OR¹⁵, —NR¹⁵R¹⁶, —C(O)NR¹⁵R¹⁶, —NR¹⁵C(O)R¹⁶, —C(O)R¹⁵, —S(O)₂R¹⁵—C(O)NR¹⁵S(O)₂R¹⁶ or C₁-C₆ alkyl optionally substituted by halogen, oxo, CN, —NH₂ or —OH;

or R¹¹ and R¹² are taken together with the atom to which they attached to form a 3- to 6-membered heterocyclyl optionally substituted by C₁-C₆ alkyl, halogen, oxo, —CN, —OR¹⁵ or —NR¹⁵R¹⁶;

or R¹² and R¹³ are taken together with the atom to which they attached to form a 3- to 6-membered heterocyclyl optionally substituted by C₁-C₆ alkyl, halogen, oxo, —CN, —NH₂ or —OH;

R¹⁵ and R¹⁶ are each independently hydrogen, C₁-C₆ alkyl, C₂-C₆ alkenyl, or C₂-C₆ alkynyl, wherein each is optionally substituted by C₁-C₆ alkyl, halogen, oxo, —CN, —NH₂ or —OH;

or R¹⁵ and R¹⁶ are taken together with the atom to which they attached to form a 3- to 6-membered heterocyclyl optionally substituted by halogen, oxo, —CN, —NH₂ or —OH;

provided that:
i) when R¹ is hydrogen and R⁴ and R⁵ are taken together with the atom to which they attached to form a 5-membered heteroaryl then R⁸ is other than 6-membered heterocyclyl; or
ii) when R³ is —CH₃ then R⁴ or R⁷ is not —NR¹¹R¹².

In some embodiments, the compound of formula (I) or a tautomer, salt, polymorph, solvate or stereoisomer thereof, is a compound of formula (II), (II-A) to (II-X), (III), (III-A) to (III-P), (IV), (IV-A) to (IV-P), (V) or (V-A) to (V-L) or a tautomer, salt, polymorph, solvate or stereoisomer thereof, as detailed herein.

In another aspect, the present invention provides method of treating a disease or disorder associated with excessive activity of transforming growth factor beta (TGF-β), particularly type 1 or activin-like kinase 5 (ALK 5) in an individual in need thereof, wherein the method comprises administering to the individual an effective amount of a compound of the present invention (collectively, a compound of formula (I), (II), (II-A) to (II-X), (III), (M-A) to (III-P), (IV), (IV-A) to (IV-P), (V) or (V-A) to (V-L)), or a tautomer, salt, polymorph, solvate or stereoisomer thereof.

In another aspect, the present invention provides method of treating cancer in an individual in need thereof, wherein the method comprises administering to the individual an effective amount of a compound of the present invention (collectively, a compound of formula (I), (II), (II-A) to (II-X), (III), (III-A) to (III-P), (IV), (IV-A) to (IV-P), (V) or (V-A) to (V-L)), or a tautomer, salt, polymorph, solvate or stereoisomer thereof.

In another aspect, the present invention provides method of treating a disease or disorder associated with excessive activity of transforming growth factor beta (TGF-β), particularly type 1 or activin-like kinase 5 (ALK 5) in an individual in need thereof, wherein the method comprises administering to the individual an effective amount of a compound of the present invention (collectively, a compound of formula (I), (II), (II-A) to (II-X), (III), (M-A) to (III-P), (IV), (IV-A) to (IV-P), (V) or (V-A) to (V-L)), or a tautomer, salt, polymorph, solvate or stereoisomer thereof in combination with another therapeutic agent.

In another aspect, the present invention also provides pharmaceutical compositions, comprising a compound of the present invention (collectively, a compound of formula (I), (II), (II-A) to (II-X), (III), (III-A) to (III-P), (IV), (IV-A) to (IV-P), (V) or (V-A) to (V-L)) and at least one pharmaceutically acceptable excipient.

In another aspect, the present invention provides method of treating a disease or disorder associated with excessive activity of transforming growth factor beta (TGF-β), particularly type 1 or activin-like kinase 5 (ALK 5) in an individual in need thereof, wherein the method comprises administering to the individual an effective amount of a pharmaceutical composition comprising a compound of the present invention (collectively, a compound of formula (I), (II), (II-A) to (II-X), (III), (III-A) to (III-P), (IV), (IV-A) to (IV-P), (V) or (V-A) to (V-L)), or a tautomer, salt, polymorph, solvate or stereoisomer thereof.

In another aspect, the present invention provides processes for preparing compounds and intermediates thereof disclosed in the present invention.

### DETAILED DESCRIPTION OF THE INVENTION

### Definitions

“Alkyl” refers to and includes saturated linear and branched univalent hydrocarbon structures and combination thereof, having the number of carbon atoms designated (i.e., C₁-C₁₀ means one to ten carbons). Particular alkyl groups are those having 1 to 20 carbon atoms (a “C₁-C₂₀ alkyl”). More particular alkyl groups are those having 1 to 8 carbon atoms (a “C₁-C₈ alkyl”), 3 to 8 carbon atoms (a “C₃-C₈ alkyl”), 1 to 6 carbon atoms (a “C₁-C₆ alkyl”), 1 to 5 carbon atoms (a “C₁-C₅ alkyl”), or 1 to 4 carbon atoms (a “C₁-C₄ alkyl”). Examples of alkyl include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like.

“Alkenyl” as used herein refers to an unsaturated linear or branched univalent hydrocarbon chain or combination thereof, having at least one site of olefinic unsaturation (i.e., having at least one moiety of the formula C═C) and having the number of carbon atoms designated (i.e., C₂-C₁₀ means two to ten carbon atoms). The alkenyl group may be in “cis” or “trans” configurations, or alternatively in “E” or “Z” configurations. Particular alkenyl groups are those having 2 to 20 carbon atoms (a “C₂-C₂₀ alkenyl”), having 2 to 8 carbon atoms (a “C₂-C₈ alkenyl”), having 2 to 6 carbon atoms (a “C₂-C₆ alkenyl”), or having 2 to 4 carbon atoms (a “C₂-C₄ alkenyl”). Examples of alkenyl include, but are not limited to, groups such as ethenyl (or vinyl), prop-1-enyl, prop-2-enyl (or allyl), 2-methylprop-1-enyl, but-1-enyl, but-2-enyl, but-3-enyl, buta-1,3-dienyl, 2-methylbuta-1,3-dienyl, homologs and isomers thereof, and the like.

“Alkylene” as used herein refers to the same residues as alkyl, but having bivalency. Particular alkylene groups are those having 1 to 6 carbon atoms (a “C₁-C₆ alkylene”), 1 to 5 carbon atoms (a “C₁-C₅ alkylene”), 1 to 4 carbon atoms (a “C₁-C₄ alkylene”) or 1 to 3 carbon atoms (a “C₁-C₃ alkylene”). Examples of alkylene include, but are not limited to, groups such as methylene (—CH₂—), ethylene (—CH₂CH₂—), propylene (—CH₂CH₂CH₂—), butylene (—CH₂CH₂CH₂CH₂—), and the like.

“Alkynyl” as used herein refers to an unsaturated linear or branched univalent hydrocarbon chain or combination thereof, having at least one site of acetylenic unsaturation (i.e., having at least one moiety of the formula CC) and having the number of carbon atoms designated (i.e., C₂-C₁₀ means two to ten carbon atoms). Particular alkynyl groups are those having 2 to 20 carbon atoms (a “C₂-C₂₀ alkynyl”), having 2 to 8 carbon atoms (a “C₂-C₈ alkynyl”), having 2 to 6 carbon atoms (a “C₂-C₆ alkynyl”), or having 2 to 4 carbon atoms (a “C₂-C₄ alkynyl”). Examples of alkynyl include, but are not limited to, groups such as ethynyl (or acetylenyl), prop-1-ynyl, prop-2-ynyl (or propargyl), but-1-ynyl, but-2-ynyl, but-3-ynyl, homologs and isomers thereof, and the like.

“Aryl” refers to and includes polyunsaturated aromatic hydrocarbon groups. Aryl may contain additional fused rings (e.g., from 1 to 3 rings), including additionally fused aryl, heteroaryl, cycloalkyl, and/or heterocyclyl rings. In one variation, the aryl group contains from 6 to 14 annular carbon atoms. Examples of aryl groups include, but are not limited to, phenyl, naphthyl, biphenyl, and the like.

“Carbonyl” refers to the group C═O.

“Cycloalkyl” refers to and includes cyclic univalent hydrocarbon structures, which may be fully saturated, mono- or polyunsaturated, but which are non-aromatic, having the number of carbon atoms designated (e.g., C₁-C₁₀ means one to ten carbons). Cycloalkyl can consist of one ring, such as cyclohexyl, or multiple rings, such as adamantly, but excludes aryl groups. A cycloalkyl comprising more than one ring may be fused, spiro or bridged, or combinations thereof. A preferred cycloalkyl is a cyclic hydrocarbon having from 3 to 13 annular carbon atoms. A more preferred cycloalkyl is a cyclic hydrocarbon having from 3 to 8 annular carbon atoms (a “C₃-C₈ cycloalkyl”). Examples of cycloalkyl include, but are not limited to, cyclopropyl, cyclobutyl, cyclopentyl, cyclohexyl, 1-cyclohexenyl, 3-cyclohexenyl, cycloheptyl, norbornyl, and the like.

“Halo” or “halogen” refers to elements of the Group 17 series having atomic number 9 to 85. Preferred halo groups include fluoro, chloro, bromo and iodo. Where a residue is substituted with more than one halogen, it may be referred to by using a prefix corresponding to the number of halogen moieties attached, e.g., dihaloaryl, dihaloalkyl, trihaloaryl etc. refer to aryl and alkyl substituted with two (“di”) or three (“tri”) halo groups, which may be but are not necessarily the same halo; thus 4-chloro-3-fluorophenyl is within the scope of dihaloaryl. An alkyl group in which each hydrogen is replaced with a halo group is referred to as a “perhaloalkyl.” A preferred perhaloalkyl group is trifluoroalkyl (—CF₃). Similarly, “perhaloalkoxy” refers to an alkoxy group in which a halogen takes the place of each H in the hydrocarbon making up the alkyl moiety of the alkoxy group. An example of a perhaloalkoxy group is trifluoromethoxy (—OCF₃).

“Heteroaryl” refers to and includes unsaturated aromatic cyclic groups having from 1 to 10 annular carbon atoms and at least one annular heteroatom, including but not limited to heteroatoms such as nitrogen, oxygen and sulfur, wherein the nitrogen and sulfur atoms are optionally oxidized, and the nitrogen atom(s) are optionally quaternized. A heteroaryl group can be attached to the remainder of the molecule at an annular carbon or at an annular heteroatom. Heteroaryl may contain additional fused rings (e.g., from 1 to 3 rings), including additionally fused aryl, heteroaryl, cycloalkyl, and/or heterocyclyl rings. Examples of heteroaryl groups include, but are not limited to, pyridyl, pyrimidyl, imidazolyl, pyrrolyl, pyrazolyl, 1,2,4-triazole, thiophenyl, furanyl, thiazolyl, isothiazolyl, 1,3,4-thiadiazolyl oxazolyl, isoxazolyl, 1,3,4-oxadiazolyl, 1,2,3-triazolyl, pyridazinyl, pyrrolopyridinyl, pyrrolopyridazinyl, pyrrolopyrimidinyl, pyrazolopyridinyl, pyrazolopyrimidinyl, dihydropyrrolopyridinyl, dihydrocyclopentapyridinyl, imidazopyridinyl, purinyl, quinolinyl, 1,8-naphthyridinyl, 1,7-naphthyridinyl, 1,6-naphthyridinyl, 1,5-naphthyridinyl, quinazolinyl, pyridopyrimidinyl, cinnolinyl and pyridopyridazinyl and the like.

“Heterocycle” or “heterocyclyl” refers to a saturated or an unsaturated non-aromatic group having from 1 to 10 annular carbon atoms and from 1 to 4 annular heteroatoms, such as nitrogen, sulfur or oxygen, and the like, wherein the nitrogen and sulfur atoms are optionally oxidized, and the nitrogen atom(s) are optionally quaternized. A heterocyclyl group may have a single ring or multiple condensed rings, but excludes heteroaryl groups. A heterocycle comprising more than one ring may be fused, spiro or bridged, or any combination thereof. In fused ring systems, one or more of the fused rings can be aryl or heteroaryl. Examples of heterocyclyl groups include, but are not limited to, aziridinyl, azetidinyl, oxetanyl, morpholinyl, thiomorpholinyl, azepanyl tetrahydropyranyl, dihydropyranyl, piperidinyl, piperazinyl, pyrrolidinyl, thiazolinyl, thiazolidinyl, tetrahydrofuranyl, tetrahydrothiophenyl, and the like.

“Oxo” refers to the moiety ═O.

“ALK” refers to activin-like kinase, these include one or more like ALK1, ALK2, ALK3, ALK4, ALK5 or ALK6. More specifically the term “ALK” refers to ALK5.

“TGF-beta” refers to transforming growth factor beta.

“Optionally substituted” unless otherwise specified means that a group may be unsubstituted or substituted by one or more (e.g., 1, 2, 3, 4 or 5) of the substituents listed for that group in which the substituents may be the same of different. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 2 to 5, 3 to 5, 2 to 3, 2 to 4, 3 to 4, 1 to 3, 1 to 4 or 1 to 5 substituents.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” or “treating” is an approach for obtaining beneficial or desired results including clinical results. For example, beneficial or desired results include, but are not limited to, one or more of the following: decreasing symptoms resulting from the disease, increasing the quality of life of those suffering from the disease, decreasing the dose of other medications required to treat the disease, delaying the progression of the disease, and/or prolonging survival of individuals. In reference to cancers or other unwanted cell proliferation, beneficial or desired results include shrinking a tumor (reducing tumor size); decreasing the growth rate of the tumor (such as to suppress tumor growth); reducing the number of cancer cells; inhibiting, retarding or slowing to some extent and preferably stopping cancer cell infiltration into peripheral organs; inhibiting (slowing to some extent and preferably stopping) tumor metastasis; inhibiting tumor growth; preventing or delaying occurrence and/or recurrence of tumor; and/or relieving to some extent one or more of the symptoms associated with the cancer. In some embodiments, beneficial or desired results include preventing or delaying occurrence and/or recurrence, such as of unwanted cell proliferation.

As used herein, “delaying development of a disease” means to defer, hinder, slow, retard, stabilize, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

As used herein, an “effective dosage” or “effective amount” of compound or salt thereof or pharmaceutical composition is an amount sufficient to effect beneficial or desired results. For prophylactic use, beneficial or desired results include results such as eliminating or reducing the risk, lessening the severity of, or delaying the onset of the disease, including biochemical, histological and/or behavioral symptoms of the disease, its complications and intermediate pathological phenotypes presenting during development of the disease. For therapeutic use, beneficial or desired results include ameliorating, palliating, lessening, delaying or decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In reference to cancers or other unwanted cell proliferation, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay development. In some embodiments, an effective amount is an amount sufficient to prevent or delay occurrence and/or recurrence. An effective amount can be administered in one or more administrations, in the case of cancer, the effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer. An effective dosage can be administered in one or more administrations. For purposes of this disclosure, an effective dosage of compound or a salt thereof, or pharmaceutical composition is an amount sufficient to accomplish prophylactic or therapeutic treatment either directly or indirectly. It is intended and understood that an effective dosage of a compound or salt thereof, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

As used herein, the term “individual” is a mammal, including humans. An individual includes, but is not limited to, human, bovine, horse, feline, canine, rodent, or primate. In some embodiments, the individual is human. The individual (such as a human) may have advanced disease or lesser extent of disease, such as low tumor burden. In some embodiments, the individual is at an early stage of a proliferative disease (such as cancer). In some embodiments, the individual is at an advanced stage of a proliferative disease (such as an advanced cancer).

Reference to “about” a value or parameter herein includes (and describes) embodiments that are directed to that value or parameter per se. For example, description referring to “about X” includes description of “X”.

It is understood that aspects and variations described herein also include “consisting” and/or “consisting essentially of” aspects and variations.

### Compounds

In one aspect, the present invention provides a compound of formula (I):

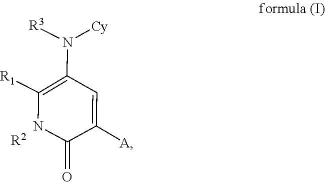

or a tautomer, salt, polymorph, solvate or stereoisomer thereof, wherein

Cy is

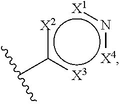

wherein

X¹ is N, CH or CR⁴;

X² is N, CH or CR⁵;

X³ is N, CH or CR⁶;

X⁴ is N, CH or CR⁷; wherein 0, 1 or 2 of X¹, X², X³ and X⁴ are N;

A is C₆ aryl, 5- to 6-membered heteroaryl, C₃-C₆ cycloalkyl or 5- to 6-membered heterocyclyl, wherein A is optionally substituted with 0 to 3 R⁹;

R¹ is independently hydrogen, halogen, —CN, C₁-C₆ alkyl, C₂-C₆ alkenyl, C₂-C₆ alkynyl, C₃-C₆ cycloalkyl, 3- to 6-membered heterocyclyl, 5- to 6-membered heteroaryl, C₁-C₃ alkoxy, C₁-C₃haloalkoxy, C₁-C₃haloalkyl, —OR¹¹, —NR¹¹R¹², —C(O)NR¹²R¹³, —C(O)R¹¹, —OC(O)R¹¹, —C(O)OR¹¹, —OC(O)NR¹²R¹³, —NR¹¹C(O)R¹², —NR¹¹C(O)OR¹², —NR¹¹C(O)NR¹²R¹³, —SR¹¹, —S(O)R¹¹, —S(O)₂R¹¹, —NR¹¹S(O)R¹², —C(O)NR¹¹S(O)R¹², —NR¹¹S(O)₂R¹², —C(O)NR¹¹S(O)₂R¹², —S(O)NR¹²R¹³, —S(O)₂NR¹²R¹³, —(C₁-C₃ alkylene)CF₃, —(C₁-C₃ alkylene)OR¹¹, (C₁-C₃ alkylene) cycloalkyl, —(C₁-C₃ alkylene)NR¹²R¹³, —(C₁—C₃ alkylene)C(O)R¹¹, —(C₁-C₃ alkylene)C(O)OR¹¹, —(C₁-C₃ alkylene)C(O)NR¹²R¹³, —(C₁-C₃ alkylene) —C(O)OR¹¹, —(C₁-C₃ alkylene)OC(O)NR¹²R¹³, —(C₁-C₃ alkylene)S(O)R¹¹ or —(C₁-C₃ alkylene)S(O)₂R¹¹, wherein each R¹ is optionally substituted with R¹⁰;

R² is hydrogen, C₁-C₆ alkyl, C₂-C₆ alkenyl, C₂-C₆ alkynyl, C₃-C₆ cycloalkyl, 3- to 6-membered heterocyclyl, C₁-C₃ alkoxy, C₁-C₃haloalkoxy, —C(O) R¹¹ or —(C₁-C₃ alkylene)OR¹¹;

or R¹ and R² are taken together with the atom to which they are attached to form a 5- to 7-membered heterocyclyl containing 1 to 3 heteroatom independently selected from N, O or S; each of which is optionally substituted by 0 to 3 R⁸;

R³ is hydrogen or C₁-C₆ alkyl;

R⁴, R⁵, R⁶ and R⁷ are independently hydrogen, halogen, oxo, —CN, C₁-C₆ alkyl, C₁-C₆ cycloalkyl, C₁-C₃ alkoxy, C₁-C₃ haloalkoxy, C₁-C₃ haloalkyl, 3- to 6-membered heterocyclyl, 5- to 6-membered heteroaryl, —NR¹¹R¹², S(O)₂NR¹²R¹³, —NR¹¹S(O)₂R¹², —C(O)NR¹²R¹³, NR¹¹C(O)R¹² (or —C(O)OR¹¹, each of which is optionally substituted by halogen, oxo, —CN, —OR¹⁵ or —NR¹⁵R¹⁶;

or R⁴ and R⁵ are taken together with the atom to which they are attached to form a 5- to 6-membered cycloalkyl or C₆ aryl; or 5- to 6-membered heterocyclyl or heteroaryl containing 1 to 3 heteroatom independently selected from N, O or S; each of which is optionally substituted by 0 to 3 R⁸;

each R⁸ is independently hydrogen, halogen, oxo, —CN, C₁-C₆ alkyl, C₁-C₆ cycloalkyl, C₁-C₃ alkoxy, C₁-C₃ haloalkoxy, C₁-C₃ haloalkyl, 3- to 6-membered heterocyclyl, —NR¹¹R¹², —C(O)NR¹²R¹³, —NR¹¹C(O)R¹², —C(O)OR¹¹, —OR¹¹; —(C₁-C₃ alkylene)NR¹¹R¹² or —(C₁-C₃ alkylene)3- to 6-membered heterocyclyl, each of which is optionally substituted by C₁-C₆ alkyl, halogen, oxo, —CN, —OR¹⁵ or —NR¹⁵R¹⁶;

each R⁹ is independently halogen, —CN, C₁-C₆ alkyl, C₃-C₆ cycloalkyl, 3- to 6-membered heterocyclyl, C₁-C₃ haloalkoxy, C₁-C₃haloalkyl, —OR¹¹, NR¹¹R¹², C(O)NR¹²R¹³, C(O)R¹¹, SR¹¹, S(O)R¹² or S(O)₂R¹¹, each of which is optionally substituted by halogen, oxo, —CN, —OR¹⁵ or —NR¹⁵R¹⁶;

R¹⁰ is independently hydrogen, halogen, oxo, —CN, C₁-C₆ alkyl, C₃-C₆ cycloalkyl, 3- to 6-membered heterocyclyl, C₁-C₃haloalkoxy, C₁-C₃ haloalkyl, —OR¹⁵, —NR¹⁵ R¹⁶, —C(O)NR¹⁵R¹⁶, —C(O)R¹⁵, —SR¹⁵, —S(O)R¹⁵ or —S(O)₂R¹⁵;

R¹¹, R¹² and R¹³ are independently hydrogen, C₁-C₆ alkyl, C₂-C₆ alkenyl, C₂-C₆ alkynyl, C₃-C₆ cycloalkyl, 3- to 6-membered heterocyclyl, 5- to 6-membered heteroaryl or —(C₁-C₃ alkylene)OR¹⁵, each of which is optionally substituted with C₃-C₆ cycloalkyl, halogen, oxo, C₁-C₃ alkoxy, 3- to 6-membered heterocyclyl, —OR¹⁵, —C(O)NR¹⁵R¹⁶, —NR¹⁵C(O)R¹⁶, —C(O)R¹⁵, —S(O)₂R¹⁵, —C(O)NR¹⁵S(O)₂R¹⁶ or C₁-C₆ alkyl optionally substituted by halogen, oxo, CN, —NH₂ or —OH;

or R¹¹ and R¹² are taken together with the atom to which they attached to form a 3- to 6-membered heterocyclyl optionally substituted by C₁-C₆ alkyl, halogen, oxo, —CN, —OR¹⁵ or —NR¹⁵R¹⁶;

or R¹² and R¹³ are taken together with the atom to which they attached to form a 3- to 6-membered heterocyclyl optionally substituted by C₁-C₆ alkyl, halogen, oxo, —CN, —NH₂ or —OH;

R¹⁵ and R¹⁶ are each independently hydrogen, C₁-C₆ alkyl, C₂-C₆ alkenyl, or C₂-C₆ alkynyl, wherein each is optionally substituted by C₁-C₆ alkyl, halogen, oxo, —CN, —NH₂ or —OH;

or R¹⁵ and R¹⁶ are taken together with the atom to which they attached to form a 3- to 6-membered heterocyclyl optionally substituted by halogen, oxo, —CN, —NH₂ or —OH;

provided that:
i) when R¹ is hydrogen and R⁴ and R⁵ are taken together with the atom to which they attached to form a 5-membered heteroaryl then R⁸ is other than 6-membered heterocyclyl; or
ii) when R³ is —CH₃ then R⁴ or R⁷ is not —NR¹¹R¹².

In some embodiments provided is a compound of formula (I):

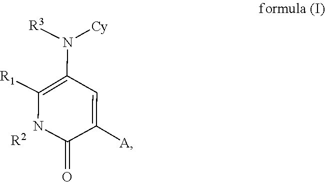

or a tautomer, salt, polymorph, solvate or stereoisomer thereof, wherein

Cy is

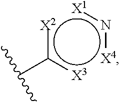

wherein

X¹ is N, CH or CR⁴;

X² is N, CH or CR⁵;

X³ is N, CH or CR⁶;

X⁴ is N, CH or CR⁷; wherein 0, 1 or 2 of X¹, X², X³ and X⁴ are N;

A is C₆ aryl, 5- to 6-membered heteroaryl, C₃-C₆ cycloalkyl or 5- to 6-membered heterocyclyl, wherein A is optionally substituted with 0 to 3 R⁹;

R¹ is independently hydrogen, halogen, —CN, C₁-C₆ alkyl, C₂-C₆ alkenyl, C₂-C₆ alkynyl, C₃-C₆ cycloalkyl, 3- to 6-membered heterocyclyl, 5- to 6-membered heteroaryl, C₁-C₃ alkoxy, C₁-C₃ haloalkoxy, C₁-C₃ haloalkyl, —OR¹¹, —NR¹¹R¹², —C(O)NR¹²R¹³, —C(O)R¹¹, —OC(O)R¹¹, —C(O)OR¹¹, —OC(O)NR¹²R¹³, —NR¹¹C(O)R¹², —NR¹¹C(O)OR¹², —NR¹¹C(O)NR¹²R¹³, —SR¹¹, —S(O)R¹¹, —S(O)₂R¹¹, —NR¹¹S(O)R¹², —C(O)NR¹¹S(O)R¹², —NR¹¹S(O)₂R¹², —C(O)NR¹¹S(O)₂R¹², —S(O)NR¹²R¹³, —S(O)₂NR¹²R¹³, —(C₁-C₃ alkylene)CF₃, —(C₁-C₃ alkylene)OR¹¹, (C₁-C₃ alkylene) cycloalkyl, —(C₁-C₃ alkylene)NR¹²R¹³, —(C₁-C₃ alkylene)C(O)R¹¹, —(C₁-C₃ alkylene)C(O)OR¹¹, —(C₁-C₃ alkylene)C(O)NR¹²R¹³, —(C₁-C₃ alkylene) —C(O)OR¹¹, —(C₁-C₃ alkylene)OC(O)NR¹²R¹³; —(C₁-C₃ alkylene)S(O)R¹¹, —(C₁-C₃ alkylene)S(O)₂R¹¹, wherein each R¹ is optionally substituted with R¹⁰;

R² is hydrogen, C₁-C₆ alkyl, C₂-C₆ alkenyl, C₂-C₆ alkynyl, C₃-C₆ cycloalkyl, 3- to 6-membered heterocyclyl, C₁-C₃ alkoxy, C₁-C₃haloalkoxy, —C(O) R¹¹ or —(C₁-C₃ alkylene)OR¹¹;

or R¹ and R² are taken together with the atom to which they are attached to form a 5- to 7-membered heterocyclyl containing 1 to 3 heteroatom independently selected from N, O or S; each of which is optionally substituted by 0 to 3 R⁸;

R³ is hydrogen or C₁-C₆ alkyl;

R⁴ and R⁷ are independently hydrogen, halogen, oxo, —CN, C₁-C₆ alkyl, C₁-C₆ cycloalkyl, C₁-C₃ alkoxy, C₁-C₃ haloalkoxy, C₁-C₃ haloalkyl, 3- to 6-membered heterocyclyl, —S(O)₂NR¹²R¹³, —NR¹¹S(O)₂R¹², —C(O)NR¹²R¹³, —NR¹¹C(O)R¹², —C(O)OR¹¹;

R⁵ and R⁶ are independently —NR¹¹R¹² or R⁴ or R⁷;

or R⁴ and R⁵ are taken together with the atom to which they are attached to form a 5- to 6-membered cycloalkyl or C₆ aryl; or 5- to 6-membered heterocyclyl or heteroaryl containing 1 to 3 heteroatom independently selected from N, O or S; each of which is optionally substituted by 0 to 3 R⁸;

each R⁸ is independently hydrogen, halogen, oxo, —CN, C₁-C₆ alkyl, C₁-C₆ cycloalkyl, C₁-C₃ alkoxy, C₁-C₃ haloalkoxy, C₁-C₃ haloalkyl, 3- to 6-membered heterocyclyl, —NR¹¹R¹², —C(O)NR¹²R¹³, —NR¹¹C(O)R¹², —C(O)OR¹¹;

each R⁹ is independently halogen, —CN, C₁-C₆ alkyl, C₃-C₆ cycloalkyl, 3- to 6-membered heterocyclyl, C₁-C₃ haloalkoxy, C₁-C₃ haloalkyl, —OR¹¹, —NR¹¹R¹², C(O)NR¹²R¹³, —C(O)R¹¹ , —SR¹¹, —S(O)R¹², —S(O)₂R¹¹;

R¹⁰ is independently hydrogen, halogen, oxo, —CN, C₁-C₆ alkyl, C₃-C₆ cycloalkyl, 3- to 6-membered heterocyclyl, C₁-C₃ haloalkoxy, C₁-C₃ haloalkyl, —OR¹⁵, —NR¹⁵ R¹⁶, —C(O)NR¹⁵R¹⁶, —C(O)R¹⁵, —SR¹⁵, —S(O)R¹⁵, —S(O)₂R¹⁵;

R¹¹, R¹² and R¹³ are independently hydrogen, C₁-C₆ alkyl, C₂-C₆ alkenyl, C₂-C₆ alkynyl, C₃-C₆ cycloalkyl or 3- to 6-membered heterocyclyl, —(C₁-C₃ alkylene)OR¹⁵, each of which is optionally substituted with C₃-C₆ cycloalkyl, oxo, C₁-C₃ alkoxy, 3- to 6-membered heterocyclyl, —OR¹⁵, —NR¹⁵R¹⁶, —C(O)NR¹⁵ R¹⁶, NR¹⁵C(O)R¹⁶, C(O)R¹⁵, S(O)₂R¹⁵, or —C(O)NR¹⁵S(O)₂R¹⁶, C₁-C₆ alkyl optionally substituted by halogen, oxo, CN, —NH₂ or —OH;

or R¹² and R¹³ are taken together with the atom to which they attached to form a 3- to 6-membered heterocyclyl optionally substituted by halogen, oxo, —CN, —NH₂ or —OH;

R¹⁵ and R¹⁶ are each independently hydrogen, C₁-C₆ alkyl, C₂-C₆ alkenyl, or C₂-C₆ alkynyl, wherein each is optionally substituted by halogen, oxo, —CN, —NH₂ or —OH;

or R¹⁵ and R¹⁶ are taken together with the atom to which they attached to form a 3- to 6-membered heterocyclyl optionally substituted by halogen, oxo, —CN, —NH₂ or —OH.

In some embodiments of a compound of formula (I), Cy is

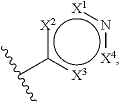

wherein

X¹ is N, CH or CR⁴;

X² is N, CH or CR⁵;

X³ is N, CH or CR⁶;

X⁴ is N, CH or CR⁷; wherein 0, 1 or 2 of X¹, X², X³ and X⁴ are N;

R⁴, R⁵, R⁶ and R⁷ are independently hydrogen, halogen, oxo, —CN, C₁-C₆ alkyl, C₁-C₆ cycloalkyl, C₁-C₃ alkoxy, C₁-C₃ haloalkoxy, C₁-C₃ haloalkyl, 3- to 6-membered heterocyclyl, 5- to 6-membered heteroaryl, —NR¹¹R¹², —S(O)₂NR¹²R¹³, NR¹¹S(O)₂R¹², C(O)NR¹²R¹³, NR¹¹C(O)R¹² or —C(O)OR¹¹, each of which is optionally substituted by halogen, oxo, —CN, —OR¹⁵ or —NR¹⁵R¹⁶;

R¹¹, R¹², R¹³, R¹⁵ and R¹⁶ are as defined herein.

provided that when R³ is —CH₃ then R⁴ or R⁷ is not —NR¹¹R¹².

In some embodiments of a compound of formula (I), Cy is

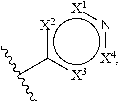

wherein X¹, X², X³, X⁴ are CH. In some embodiments of a compound of formula (I), Cy is

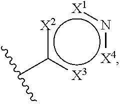

wherein X¹, X², X⁴ are CH and X³ is N. In some embodiments of a compound of formula (I), Cy is

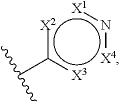

wherein X¹, X², X³ are CH and X⁴ is N.

In some embodiments of a compound of formula (I), Cy is

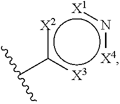

wherein X², X³, X⁴ are CH and X¹ is CR⁴. In some embodiments of a compound of formula (I), Cy is

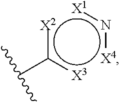

wherein X², X⁴ are CH; X³ is N and X¹ is CR⁴. In some embodiments of a compound of formula (I), Cy is

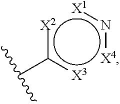

wherein X², X³ are CH; X⁴ is N and X² is CR⁴.

In some embodiments of a compound of formula (I), Cy is

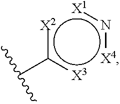

wherein X¹, X³, X⁴ are CH and X² is CR⁵. In some embodiments of a compound of formula (I), Cy is

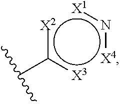

wherein X¹, X⁴ are CH; X³ is N and X² is CR⁵. In some embodiments of a compound of formula (I), Cy is

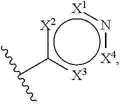

wherein X¹, X³ are CH; X⁴ is N and X² is CR⁵.

In some embodiments of a compound of formula (I), Cy is an optionally substituted 6-membered heteroaryl selected from pyridazinyl, pyridyl or pyrimidyl.

In some embodiments of a compound of formula (I), Cy is selected from the group consisting of:

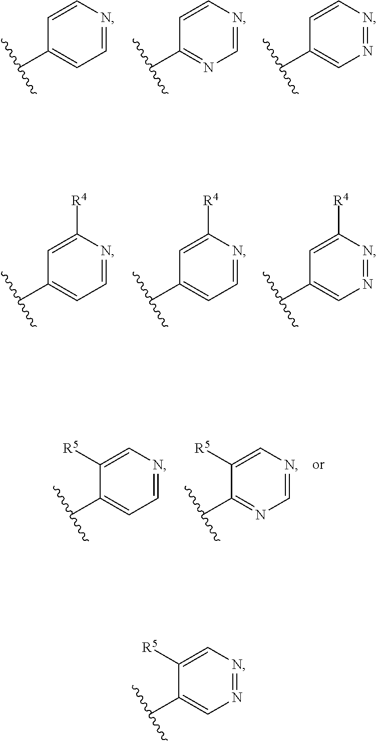

wherein the wavy lines denote attachment points; R⁴ and R⁵ are as defined herein.

In some embodiments of a compound of formula (I), Cy is

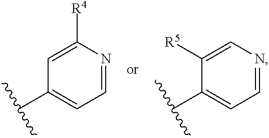

wherein the wavy lines denote attachment points; R⁴ and R⁵ are as defined herein.

In some embodiments of a compound of formula (I), R⁴ is selected from the group consisting of: —NH₂,

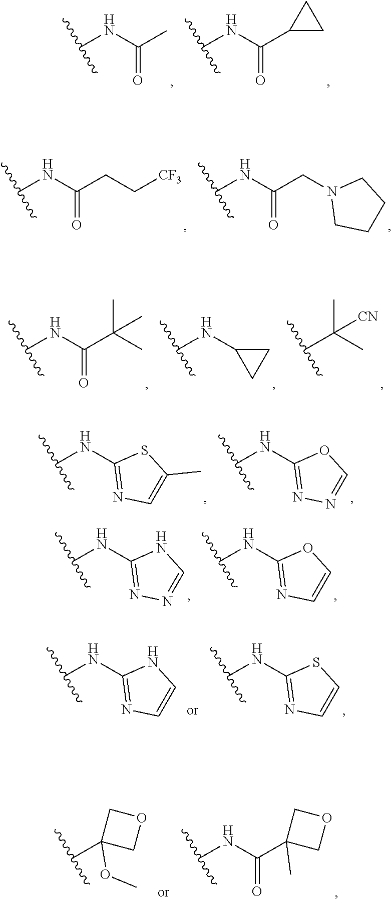

wherein the wavy lines denote attachment points.

In some embodiments of a compound of formula (I), R⁴ is

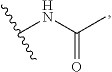 [SMILES: *NC(C)=O]

In some embodiments of a compound of formula (I), R⁵ is selected from the group consisting of:

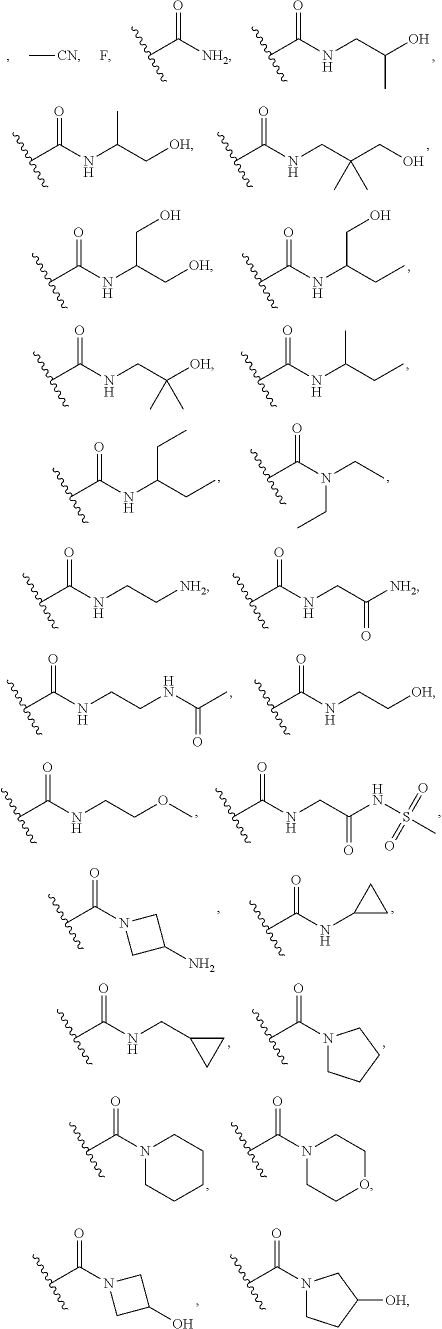

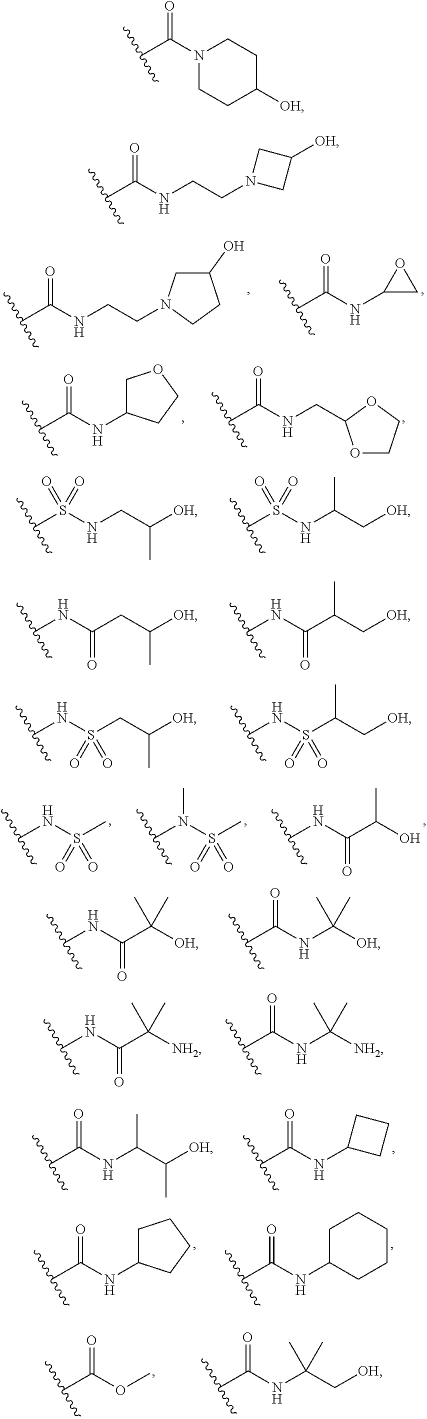

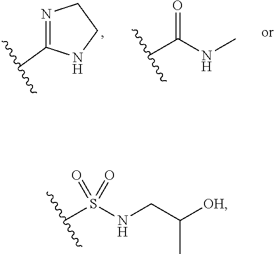

wherein the wavy lines denote attachment points.

In some embodiments of a compound of formula (I), R⁵ is selected from the group consisting of:

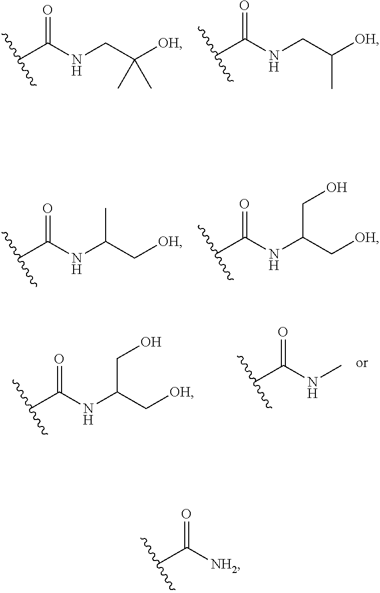

wherein the wavy lines denote attachment points.

In some embodiments of a compound of formula (I), R⁵ is

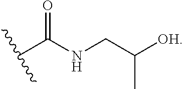 [SMILES: *C(=O)NCC(C)O]

In some embodiments of a compound of formula (I), R⁵ is

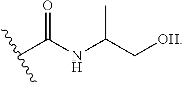 [SMILES: *C(=O)NC(C)CO]

In some embodiments of a compound of formula (I), R⁵ is

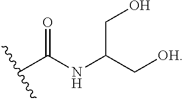 [SMILES: *C(=O)NC(CO)CO]

In some embodiments of a compound of formula (I), R⁵ is

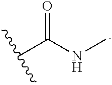 [SMILES: *C(=O)NC]

In some embodiments of a compound of formula (I), R⁵ is

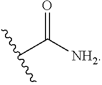 [SMILES: *C(N)=O]

In some embodiments of a compound of formula (I), Cy is

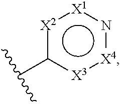

wherein

X¹ is CR⁴;

X² is CR⁵;

X³ is N, CH or CR⁶;

X⁴ is N, CH or CR⁷; wherein 0, 1 or 2 of X¹, X², X³ and X⁴ are N;

R⁴ and R⁵ are taken together with the atom to which they attached to form a 5-membered cycloalkyl; or 5-membered heterocyclyl or heteroaryl containing 1 to 3 heteroatom independently selected from N, O or S; each of which is optionally substituted by 0 to 3 R⁸;

R⁶, R⁷ and R⁸ are as defined herein.

provided that when R¹ is hydrogen and R⁴ and R⁵ are taken together with the atom to which they attached to form a 5-membered heteroaryl then R⁸ is other than 6-membered heterocyclyl.

In some embodiments of a compound of formula (I), Cy is

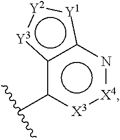

wherein

X³ is N, CH or CR⁶;

X⁴ is N, CH or CR⁷;

Y¹ and Y³ are independently N, NH, NR⁸, CH or CR⁸, S, O;

Y² is N, CH or CR⁸;

R⁶, R⁷ and R⁸ are as defined herein.

In some embodiments of a compound of formula (I), Cy is

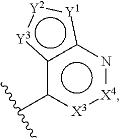

wherein X³, X⁴, Y² and Y³ are CH; and Y¹ is NH. In some embodiments of a compound of formula (I), Cy is

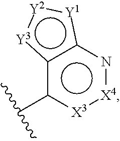

wherein X³, X⁴, Y¹ and Y² are CH; and Y³ is NH. In some embodiments of a compound of formula (I), Cy is

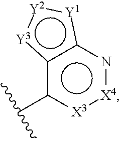

wherein X³, X⁴ and Y³ are CH; Y¹ is NH; and Y² is N. In some embodiments of a compound of formula (I), Cy is

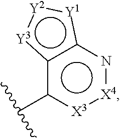

wherein X³, X⁴ and Y² are CH; Y¹ is NH; and Y³ is N. In some embodiments of a compound of formula (I), Cy is

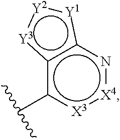

wherein X³, X⁴ and Y¹ are CH; Y² is N; and Y³ is NH. In some embodiments of a compound of formula (I), Cy is

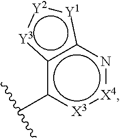

wherein X³, Y² and Y³ are CH; X⁴ is N; and Y¹ is NH. In some embodiments of compound of formula (I), Cy is

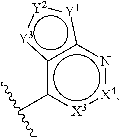

wherein X³, Y¹ and Y² are CH; X⁴ is N; and Y³ is NH. In some embodiments of compound of formula (I), Cy is

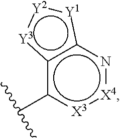

wherein X³ and Y³ are CH; X⁴ and Y² is N; and Y¹ is NH. In some embodiments of a compound of formula (I), Cy is

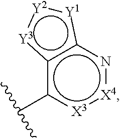

wherein X³ and Y² are CH; X⁴ and Y³ is N; and Y¹ is NH. In some embodiments of a compound of formula (I), Cy is

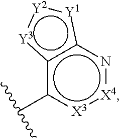

wherein X³, X⁴ and Y² are CH; Y¹ is NH and Y³ is CR⁸. In some embodiments of a compound of formula (I), Cy is

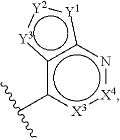

wherein X⁴, Y² and Y³ are CH; Y¹ is NH and X³ is CR⁶. In some embodiments of a compound of formula (I), Cy is

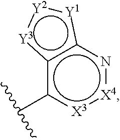

wherein X³, X⁴, and Y² are CH; Y¹ is NH and Y³ is CR⁸.

In some embodiments of a compound of formula (I), Cy is an optionally substituted pyrrolopyridinyl, pyrrolopyridazinyl, pyrrolopyrimidinyl, pyrazolopyridinyl, pyrazolopyrimidinyl, dihydropyrrolopyridinyl, imidazopyridinyl or purinyl.

In some embodiments of a compound of formula (I), Cy is selected from the group consisting of

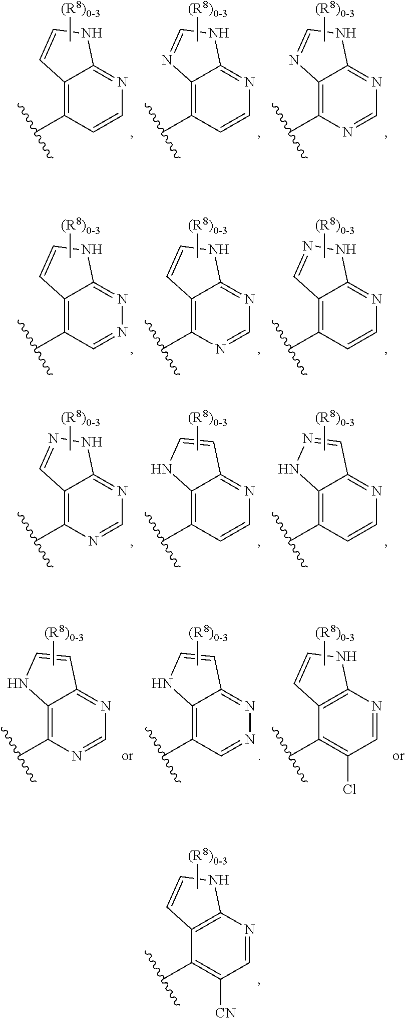

wherein the wavy lines denote attachment points; R⁸ is as defined herein.

In some embodiments of a compound of formula (I), Cy is selected from the group consisting of

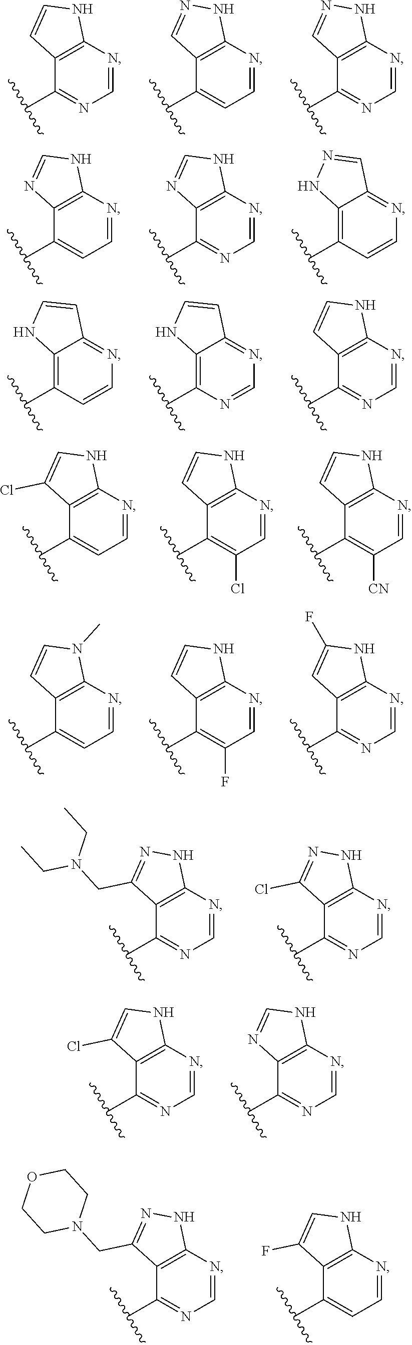

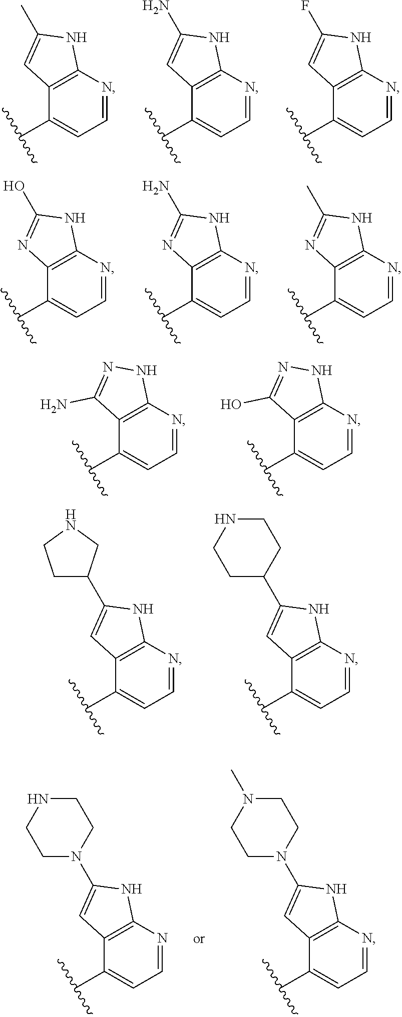

wherein the wavy lines denote attachment points.

In some embodiments of a compound of formula (I), Cy is

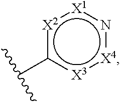

wherein

X¹ is CR⁴;

X² is CR⁵;

X³ is N, CH or CR⁶;

X⁴ is N, CH or CR⁷; wherein 0, 1 or 2 of X¹, X², X³ and X⁴ are N;

R⁴ and R⁵ are taken together with the atom to which they attached to form a 6-membered cycloalkyl or C₆ aryl; or 6-membered heterocyclyl or heteroaryl containing 1 to 3 heteroatom independently selected from N, O or S; each of which is optionally substituted by 0 to 3 R⁸;

R⁶, R⁷ and R⁸ are as defined herein.

In some embodiments of a compound of formula (I), Cy is

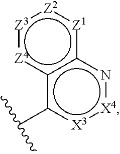

wherein

X³ is N, CH or CR⁶;

X⁴ is N, CH or CR⁷;

Z¹, Z², Z³ and Z⁴ are independently N, CH or CR⁸;

R⁶, R⁷ and R⁸ are as defined herein.

In some embodiments of a compound of formula (I), Cy is

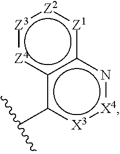

wherein X³, X⁴, Z¹, Z², Z³ and Z⁴ are CH. In some embodiments of a compound of formula (I), Cy is

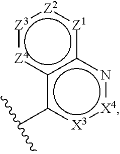

wherein X³, X⁴, Z², Z³ and Z⁴ are CH; and Z¹ is N. In some embodiments of a compound of Formula (I), Cy

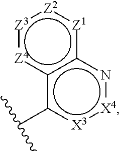

is wherein X³, X⁴, Z¹, Z³ and Z⁴ are CH; and Z² is CR⁸. In some embodiments of a compound of formula (I), Cy is

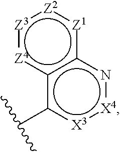

wherein X³, X⁴, Z¹, Z² and Z⁴ are CH; and Z³ is CR⁸. In some embodiments of a compound of Formula (I), Cy

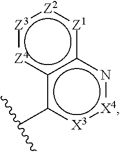

is wherein X⁴, Z¹, Z², Z³ and Z⁴ are CH; and X³ is CR⁶. In some embodiments of a compound of Formula (I), Cy is

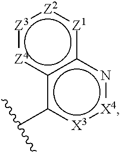

wherein X³, X⁴, Z¹, Z² and Z⁴ are CH; and Z³ is N. In some embodiments of a compound of Formula (I), Cy is

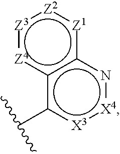

wherein X⁴, Z¹, Z² and Z⁴ are CH; and X³ and Z³ is N.

In some embodiments of a compound of formula (I), Cy is an optionally substituted quinolinyl, 1,8-naphthyridinyl, 1,7-naphthyridinyl, 1,6-naphthyridinyl, 1,5-naphthyridinyl, quinazolinyl, pyridopyrimidinyl, cinnolinyl or pyridopyridazinyl.

In some embodiments of a compound of formula (I), Cy is selected from the group consisting of

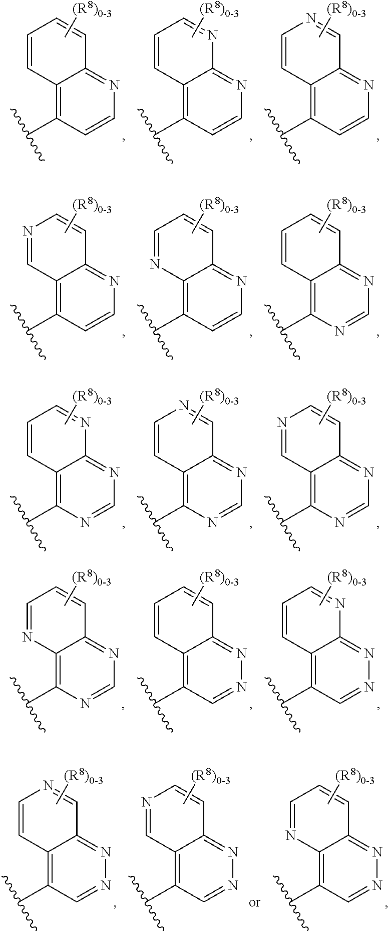

wherein the wavy lines denote attachment points; R⁸ is as defined herein.

In some embodiments of a compound of formula (I), Cy is selected from the group consisting of

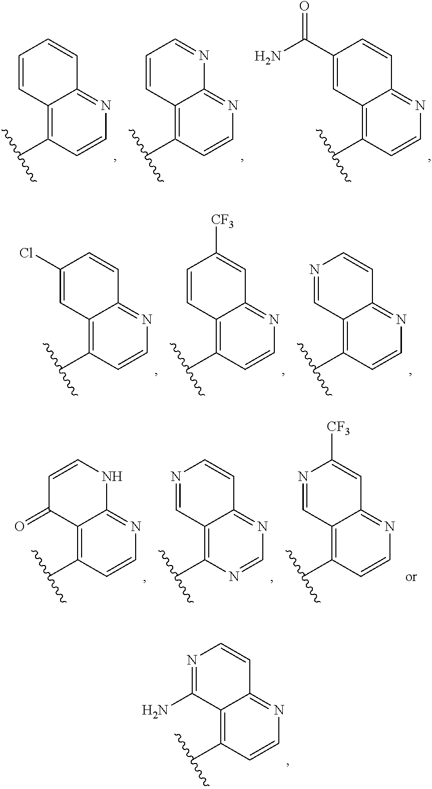

wherein the wavy lines denote attachment points.

In some embodiments of a compound of formula (I), Cy is

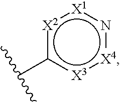

wherein

X¹ is CR⁴;

X² is CR⁵;

X³ is N, CH or CR⁶;

X⁴ is N, CH or CR⁷; wherein 0, 1 or 2 of X¹, X², X³ and X⁴ are N;

R⁴ and R⁵ are taken together with the atom to which they attached to form a 5 to 6-membered cycloalkyl or 5 to 6-membered heterocyclyl containing 1 to 3 heteroatom independently selected from N, O or S; each of which is optionally substituted by 0 to 3 R⁸;

R⁶, R⁷ and R⁸ are as defined herein.

In some embodiments of a compound of formula (I), Cy is

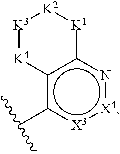

wherein

X³ is N, CH or CR⁶;

X⁴ is N, CH or CR⁷;

K¹ is S, O, NH, NR⁸, CH₂, CHR⁸, C═O or CR⁸R^{8′};

K² is NH, NR⁸, CH₂, CHR⁸, C═O or CR⁸R^{8′};

K³ is NH, NR⁸, CH₂, CHR⁸, C═O or CR⁸R^{8′} or absent;

K⁴ is S, O, NH, NR⁸, CH₂, CHR⁸, C═O or CR⁸R^{8′};

provided that:

i) no more than two of K¹, K², K³ and K⁴ are NH or NR⁸;

ii) if K³ is absent, then at least one of K¹ and K⁴ is not O or S;

iii) no more than two of K¹, K², K³ and K⁴ is C═O;

R⁸ and R^{8′} are independently hydrogen, halogen, oxo, —CN, C₁-C₆ alkyl, C₁-C₆ cycloalkyl, C₁-C₃ alkoxy, C₁-C₃ haloalkoxy, C₁-C₃ haloalkyl, 3- to 6-membered heterocyclyl, NR¹¹R¹² C(O)NR¹²R¹³, —NR¹¹C(O)R¹², —C(O)OR¹¹, —OR¹¹, —(C₁-C₃ alkylene)NR¹¹R¹² or —(C₁-C₃ alkylene)3- to 6-membered heterocyclyl, each of which is optionally substituted by C₁-C₆ alkyl, halogen, oxo, —CN, —OR¹⁵ or —NR¹⁵R¹⁶;

R¹¹, R¹², R¹³, R¹⁵ and R¹⁶ are as defined herein.

In some embodiments of a compound of formula (I), Cy is

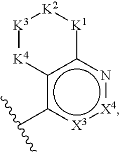

wherein X³ is N, X⁴ is CH, K¹ is NH, K² is C═O, K³ is absent and K⁴ is CH₂. In some embodiments of a compound of formula (I), Cy is

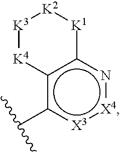

wherein X³ and X⁴ are CH, K¹ is NH, K² is C═O, K³ is absent and K⁴ is CH₂. In some embodiments of a compound of formula (I), Cy is

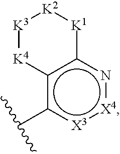

wherein X³ and X⁴ are CH, K¹ is NH, K² is C═O, K³ is absent and K⁴ is CR⁸R^{8′}.

In some embodiments of a compound of formula (I), Cy is selected from the group consisting of

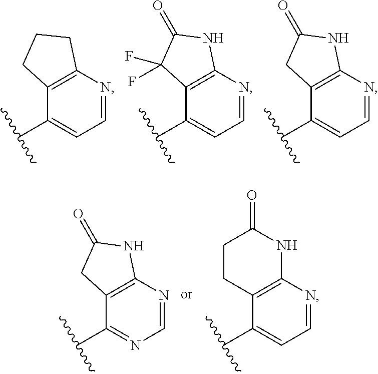

wherein the wavy lines denote attachment points.

In some embodiments of a compound of formula (I), Cy is

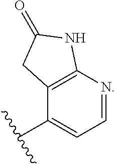 [SMILES: *c1ccnc2c1CC(=O)N2]

In some embodiments of a compound of formula (I), Cy is selected from the group consisting of

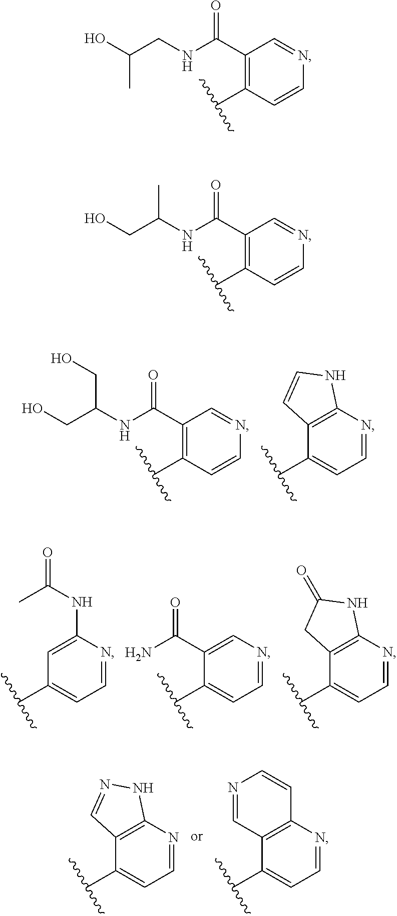

wherein the wavy lines denote attachment points.

It is understood that each description of Cy may be combined with each description of R¹, R² and R³ the same as if each and every combination were specifically and individually listed. It is similarly understood that each description of Cy may be combined with each description of A (and further with each description of R¹, R² and/or R³) the same as if each and every combination were specifically and individually listed. For example, in one aspect, it is understood that each description of Cy may be combined in one aspect with a variation in which R¹ is isopropyl, R² is hydrogen and R³ is hydrogen. In one such variation, Cy is as defined in any variation herein, R¹ is isopropyl, R² is hydrogen, R³ is hydrogen and A is phenyl or pyridyl optionally substituted by 0 to 3 R⁹. In another variation, Cy is as defined in any variation herein, R¹, R² and R³ are as defined in any variation herein and A is phenyl or pyridyl optionally substituted by 0 to 3 R⁹.

In some embodiments of a compound of formula (I), A is C₆ aryl, 5- to 6-membered heteroaryl, C₃-C₆ cycloalkyl or 5- to 6-membered heterocyclyl, wherein A is optionally substituted with 0 to 3 R⁹ groups which may be same or different. In some embodiments of a compound of formula (I), A is C₆ aryl optionally substituted with 0 to 3 R⁹ groups which may be same or different. In some embodiments of a compound of formula (I), A is phenyl optionally substituted with 0 to 3 R⁹ groups which may be same or different. In some embodiments of a compound of formula (I), A is 5- to 6-membered heteroaryl optionally substituted with 0 to 3 R⁹ groups which may be same or different. In some embodiments of a compound of formula (I), A is a 5-membered heteroaryl selected from the group consisting of furanyl, oxazolyl, thiophenyl, pyrazolyl, isoxazolyl, 1,3,4-oxadiazolyl, imidazolyl, thiazolyl, isothiazolyl, and 1,3,4-thiadiazolyl, each of which is optionally substituted with 0 to 3 R⁹ groups which may be same or different. In some embodiments of a compound of formula (I), A is a 6-membered heteroaryl optionally substituted with 0 to 3 R⁹ groups which may be same or different. In some embodiments of a compound of formula (I), A is pyridyl or pyrimidinyl, each of which is optionally substituted with 0 to 3 R⁹ groups which may be same or different.

In some embodiments of a compound of formula (I), A is optionally substituted with 0 to 3 R⁹ groups which may be same or different. In some embodiments of a compound of formula (I), A is unsubstituted. In some embodiments of a compound of formula (I), A is substituted with one R⁹. In some embodiments of a compound of formula (I), A is substituted with two R⁹. In some embodiments of a compound of formula (I), A is substituted with three R⁹. Each R⁹ is independently selected from the group consisting of halogen, —CN, C₁-C₆ alkyl, C₃-C₆ cycloalkyl, 3- to 6-membered heterocyclyl, C₁-C₃ haloalkoxy, C₁-C₃ haloalkyl, —OR¹¹, —NR¹¹ R¹², C(O)NR¹²R¹³, —C(O)R^{1′}, —SR¹¹, —S(O)R¹² or —S(O)₂R¹¹, each of which is optionally substituted by halogen, oxo, —CN, —OR¹⁵ or —NR¹⁵R¹⁶. In some embodiments of a compound of formula (I), each R⁹ is independently selected from the group consisting of halogen, C₁-C₆ alkyl, C₁-C₃ haloalkoxy, C₁-C₃ haloalkyl, —CN, —OR¹¹, —NR¹¹R¹², C(O)NR¹²R¹³. In some embodiments of a compound of formula (I), each R⁹ is independently selected from the group consisting of Cl, F, —CH₃, —NHCH₃, —CN, —C(O)NH₂, —CF₃, —OCH₃—OCF₃ or —CH₂OH. In some embodiments of a compound of formula (I), A is phenyl ring optionally substituted with 0 to 3 R⁹ groups, each R⁹ is independently selected from the group consisting of halogen, —CN, C₁-C₆ alkyl, C₃-C₆ cycloalkyl, 3- to 6-membered heterocyclyl, C₁-C₃ haloalkoxy, C₁-C₃ haloalkyl, —OR¹¹, —NR¹¹R¹², —C(O) NR¹² R¹³, —C(O) R¹¹, —SR¹¹, —S(O)R¹² or —S(O)₂R¹¹, each of which is optionally substituted by halogen, oxo, —CN, —OR¹⁵ or —NR¹⁵R¹⁶. In some embodiments of a compound of formula (I), each R⁹ is independently selected from the group consisting of halogen, C₁-C₆ alkyl, C₁-C₃ haloalkoxy, C₁-C₃ haloalkyl, —CN, —OR¹¹, —NR¹¹R¹², —C(O)NR¹²R¹³. In some embodiments of a compound of formula (I), each R⁹ is independently selected from the group consisting of Cl, F, —CH₃, —NHCH₃, —CN, —C(O)NH₂, —CF₃, —OCH₃, —OCF₃ or —CH₂OH. In some embodiments of a compound of formula (I), A is unsubstituted. In some embodiments of a compound of formula (I), A is substituted with one R⁹. In some embodiments of a compound of formula (I), A is substituted with two R⁹. In some embodiments of a compound of formula (I), A is substituted with three R⁹.

In some embodiments of a compound of formula (I), A is

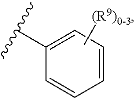

wherein each R⁹ may be the same or different. In some embodiments of a compound of formula (I), A is selected from the group consisting of

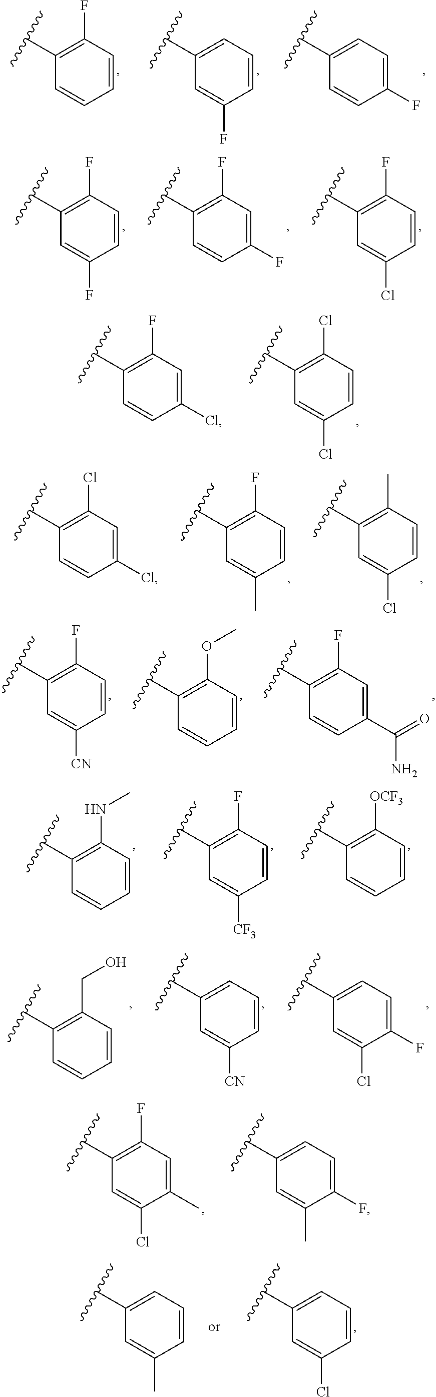

wherein wavy lines denotes point of attachment.

In some embodiments of a compound of formula (I), A is 5-membered heteroaryl optionally substituted with 0 to 3 R⁹ groups which may be same or different. In some embodiments of a compound of formula (I), A is selected from the group consisting of

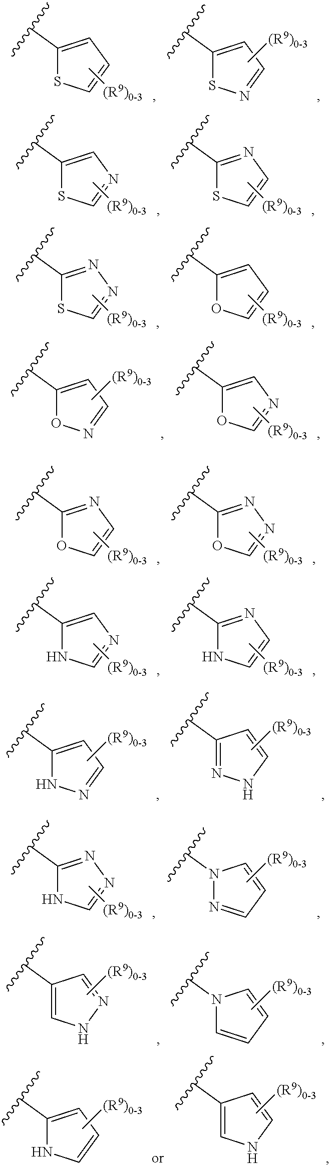

wherein the wavy lines denote attachment points and each R⁹ may be the same or different. In some embodiments of a compound of formula (I), R⁹ is methyl.

In some embodiments of a compound of formula (I), A is selected from the group consisting of

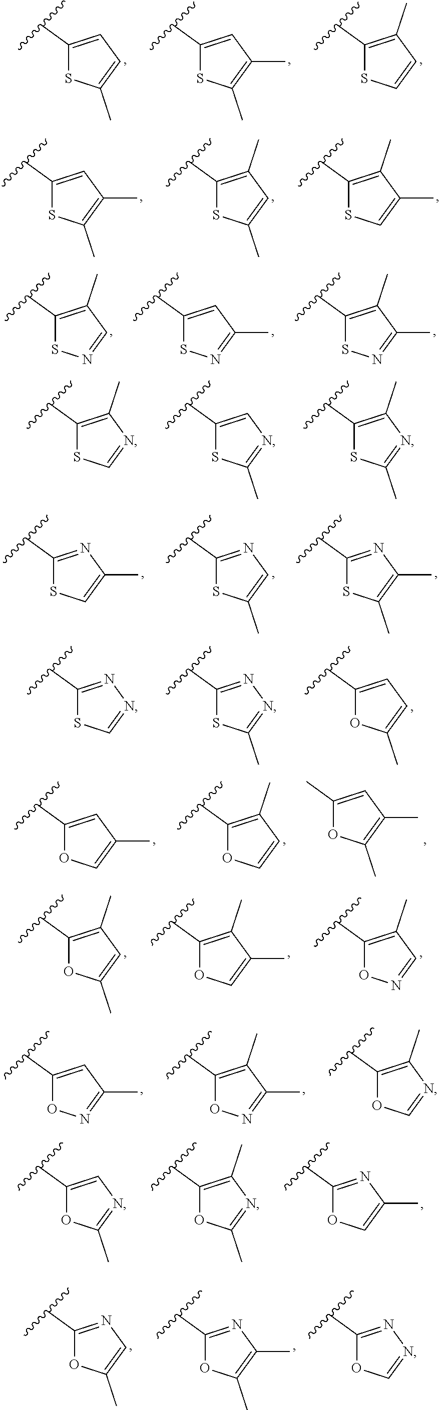

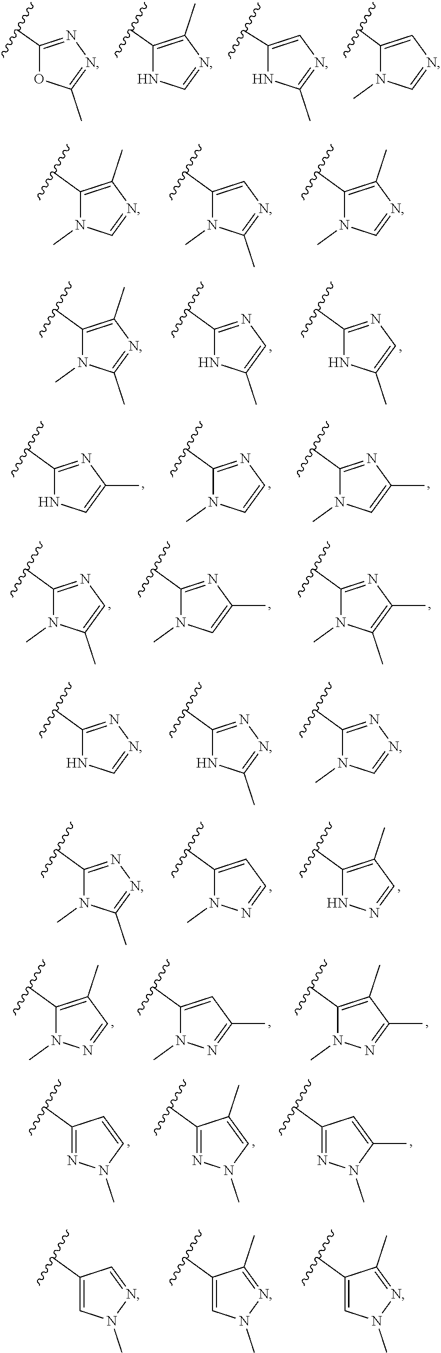

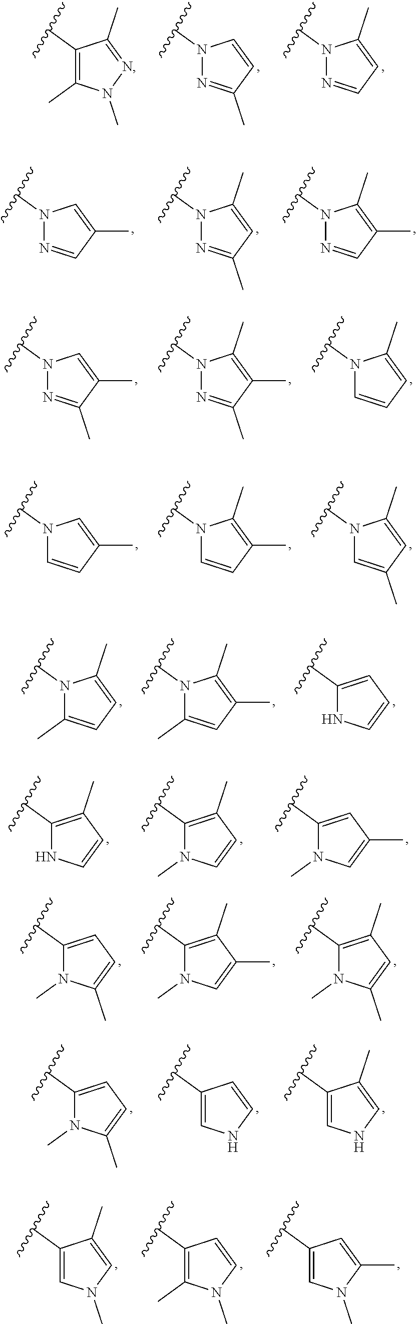

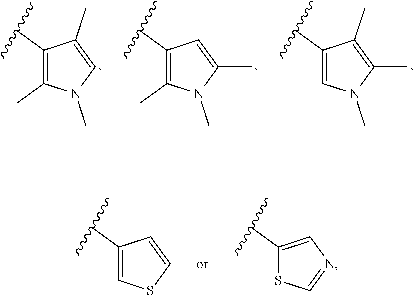

wherein the wavy lines denote attachment points.

In some embodiments of a compound of formula (I), A is 6-membered heteroaryl optionally substituted with 0 to 3 R⁹ groups which may be same or different. In some embodiments of a compound of formula (I), A is a pyridyl or pyrimidyl optionally substituted with 0 to 3 R⁹ groups which may be same or different. In some embodiments of a compound of formula (I), wherein each R⁹ is independently selected from the group consisting of Cl, F, —CH₃, —NHCH₃, —CN, —C(O)NH₂, —CF₃, —OCH₃, —OCF₃.

In some embodiments of a compound of formula (I), A is

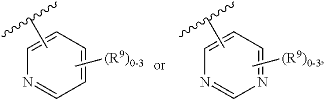

each R⁹ may be the same or different. In some embodiments of a compound of formula (I), A is selected from the group consisting of

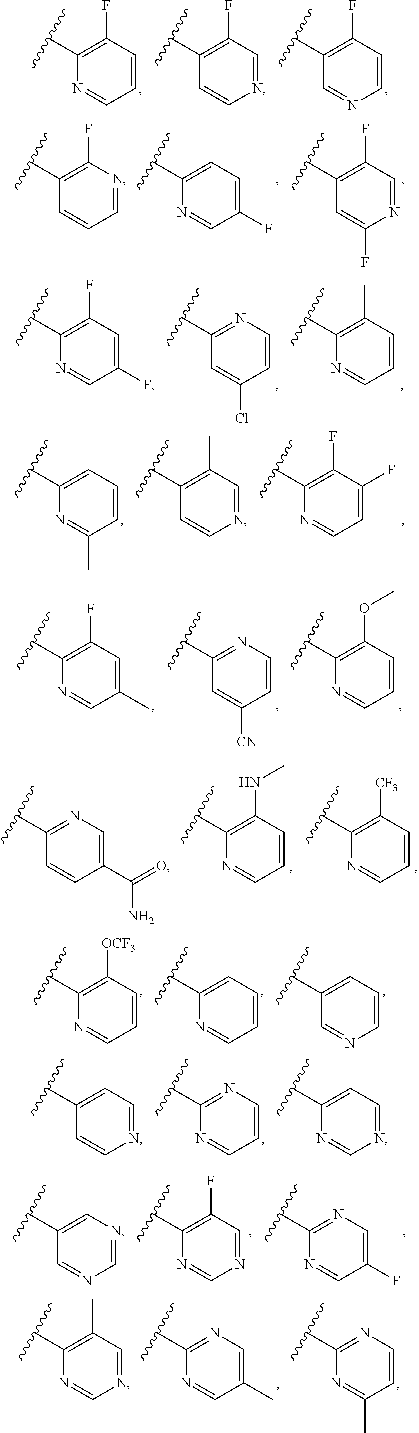

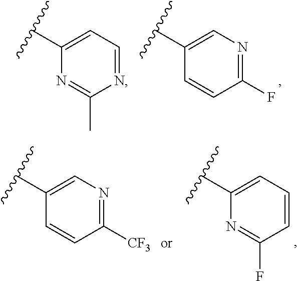

wherein the wavy lines denote attachment points.

In some embodiments of a compound of formula (I), A is selected from the group consisting of

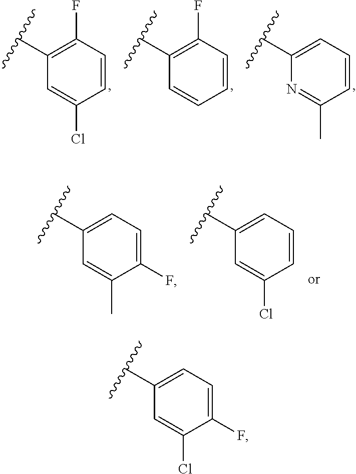

wherein the wavy lines denote attachment points.

In some embodiments of a compound of formula (I), A is

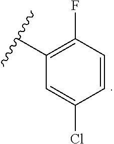 [SMILES: *c1cc(Cl)ccc1F]

In some embodiments of a compound of formula (I), A is

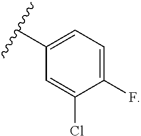 [SMILES: *c1ccc(F)c(Cl)c1]

In some embodiments of a compound of formula (I), A is

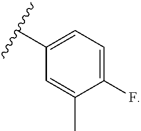 [SMILES: *c1ccc(F)c(C)c1]

In some embodiments of a compound of formula (I), A is

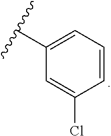 [SMILES: *c1cccc(Cl)c1]

It is understood that each description of A may be combined with each description of R¹, R² and/or R³ the same as if each and every combination were specifically and individually listed. It is similarly understood that each description of A may be combined with each description of Cy (and further with each description of R¹, R² and/or R³) the same as if each and every combination were specifically and individually listed. For example, in one aspect, it is understood that each description of A may be combined in one aspect with a variation in which R¹ is isopropyl, R² is hydrogen and R³ is hydrogen. In one such variation, A is as defined in any variation herein, R¹ is isopropyl, R² is hydrogen, R³ is hydrogen and Cy is optionally substituted pyridyl or pyrrolopyridinyl.

In some embodiments of a compound of formula (I), R¹ is independently hydrogen, halogen, —CN, C₁-C₆ alkyl, C₂-C₆ alkenyl, C₂-C₆ alkynyl, C₃-C₆ cycloalkyl, 3- to 6-membered heterocyclyl, 5- to 6-membered heteroaryl, C₁-C₃ alkoxy, C₁-C₃ haloalkoxy, C₁-C₃ haloalkyl, —OR, —NR¹¹R¹², —C(O)NR¹²R¹³, —C(O)R¹¹, —OC(O)R¹¹, —C(O)OR¹¹, —OC(O)NR¹²R¹³, —NR¹¹C(O)R¹², —NR¹¹C(O)OR¹², —NR¹¹C(O)NR¹²R¹³, —SR¹¹, —S(O)R¹¹, —S(O)₂R¹¹, —NR¹¹S(O)R¹², —C(O)NR¹¹S(O)R¹², —NR¹¹S(O)₂R¹², —C(O)NR¹¹S(O)₂R¹², —S(O)NR¹²R¹³, —S(O)₂NR¹²R¹³, —(C₁-C₃ alkylene)CF₃, —(C₁-C₃ alkylene)OR¹¹, (C₁-C₃ alkylene)cycloalkyl, —(C₁-C₃ alkylene)NR¹²R¹³, —(C₁-C₃ alkylene)C(O)R¹¹, —(C₁-C₃ alkylene)C(O)OR¹¹, —(C₁-C₃ alkylene)C(O)NR¹²R¹³, —(C₁-C₃ alkylene) —C(O)OR¹¹, —(C₁-C₃ alkylene)OC(O)NR¹²R¹³, —(C₁-C₃ alkylene)S(O)R¹¹, —(C₁-C₃ alkylene)S(O)₂R¹¹, wherein each R¹ is independently optionally substituted with R¹⁰. In some embodiments of a compound of formula (I), R¹ is hydrogen. In some embodiments of a compound of formula (I), R¹ is C₁-C₆ alkyl optionally substituted with R¹⁰. In some embodiments of a compound of formula (I), R¹ is methyl, ethyl or isopropyl, each of which is optionally substituted with R¹⁰. In some embodiments of a compound of formula (I), R¹ is C₃-C₆ cycloalkyl optionally substituted with R¹⁰. In some embodiments of a compound of formula (I), R¹ is cyclopropyl, cyclobutyl, cyclopentyl or cyclohexyl, each of which is optionally substituted with R⁶. In some embodiments of a compound of formula (I), R¹ is 3- to 6-membered heterocyclyl optionally substituted with R¹⁰. In some embodiments of a compound of formula (I), R¹ is aziridinyl, azetidinyl, oxetanyl, morpholinyl, thiomorpholinyl, piperidinyl, piperazinyl, pyrrolidinyl or azepanyl, each of which is optionally substituted with R¹⁰. In some embodiments of a compound of formula (I), R¹ is 5- to 6-membered heteroaryl optionally substituted with R¹⁰. In some embodiments of a compound of formula (I), R¹ is imidazolyl or pyrrolyl, each of which is optionally substituted with R¹⁰. In some embodiments of a compound of formula (I), R¹ is ethyl, isopropyl, cyclopropyl. cyclobutyle, —CN, —C₁, —OCH₃, —OCF₃, CF₃, —N(CH₃)₂, —CONH₂, —CH₂NH₂ or —SO₂CH₃ or —SCH₃.

In some embodiments of a compound of formula (I), R¹ is ethyl. In some embodiments of a compound of formula (I), R¹ is isopropyl. In some embodiments of a compound of formula (I), R¹ is cyclopropyl. In some embodiments of a compound of formula (I), R¹ is —N(CH₃)₂. In some embodiments of a compound of formula (I), R¹ is —OCH₃.

It is understood that each description of R¹ may be combined with each description of R², R³, A and Cy the same as if each and every combination of R¹ with R², R³, A and Cy were specifically and individually listed.

In some embodiments of a compound of formula (I), R² is independently hydrogen, C₁-C₆ alkyl, C₂-C₆ alkenyl, C₂-C₆ alkynyl, C₃-C₆ cycloalkyl, 3- to 6-membered heterocyclyl, C₁-C₃ alkoxy, C₁-C₃ haloalkoxy, —C(O) R¹¹ or —(C₁-C₃ alkylene)OR¹¹. In some embodiments of a compound of formula (I), R² is hydrogen. In some embodiments of a compound of formula (I), R² is C₁-C₆ alkyl. In some embodiments of a compound of formula (I), R² is methyl.

It is understood that each description of R² may be combined with each description of R¹, R³, A and Cy the same as if each and every combination of R² with R¹, R³, A and Cy were specifically and individually listed.

In some embodiments of a compound of formula (I), wherein R¹ and R² are taken together with the atom to which they attached to form a 5- to 7-membered heterocyclyl containing 1 to 3 heteroatom independently selected from N, O or S; each of which is optionally substituted by 0 to 3 R⁸. In some embodiments of a compound of formula (I), R¹ and R² are taken together to form

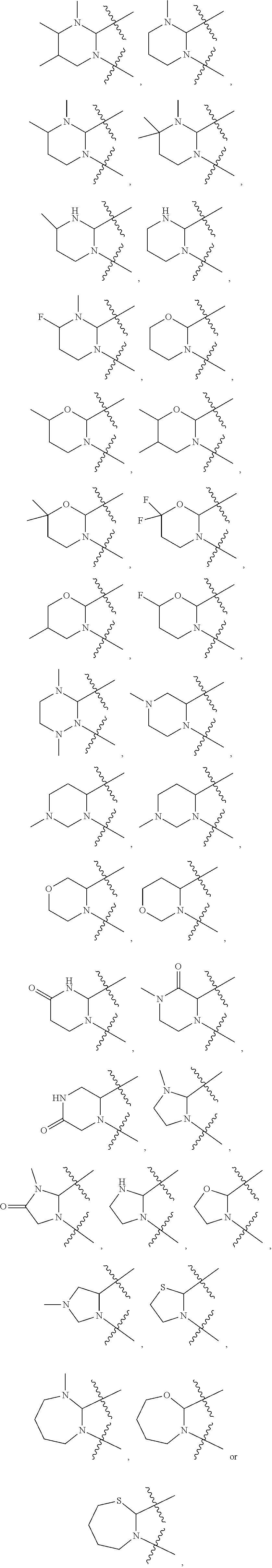

wherein the wavy lines denote attachment points.

In some embodiments of a compound of formula (I), R¹ and R² are taken together to form

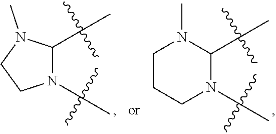

wherein the wavy lines denote attachment points.

It is understood that each description of a ring formed by taking together R¹ and R² as described herein may be combined with each description of, R³, A and Cy the same as if each and every combination of such ring with R³, A and Cy were specifically and individually listed.

In some embodiments of a compound of formula (I), wherein R³ is hydrogen. In some embodiments of a compound of formula (I), wherein R³ is C₁-C₆ alkyl. In some embodiments of a compound of formula (I), wherein R³ is —CH₃.

In some embodiments, provided is a compound of formula (II):

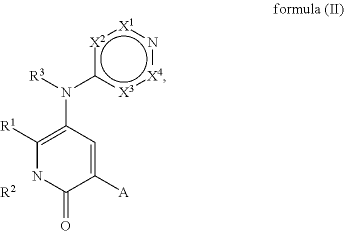

or a tautomer, salt, polymorph, solvate or stereoisomer thereof, wherein

X¹ is N, CH or CR⁴;

X² is N, CH or CR⁵;

X³ is N, CH or CR⁶;

X⁴ is N, CH or CR⁷; wherein 0, 1 or 2 of X¹, X², X³ and X⁴ are N;

R⁴, R⁵, R⁶ and R⁷ are independently hydrogen, halogen, oxo, —CN, C₁-C₆ alkyl, C₁-C₆ cycloalkyl, C₁-C₃ alkoxy, C₁-C₃ haloalkoxy, C₁-C₃ haloalkyl, 3- to 6-membered heterocyclyl, 5- to 6-membered heteroaryl, —NR¹¹R¹², —S(O)₂NR¹²R¹³, —NR¹¹S(O)₂R¹², —C(O)NR¹²R¹³, —NR¹¹C(O)R¹², —C(O)OR¹¹, each of which is optionally substituted by halogen, oxo, —CN, —OR¹⁵ or —NR¹⁵R¹⁶;

A, R¹, R², R³, R¹¹, R¹², R¹³, R¹⁵ and R¹⁶ are as defined herein. provided that when R³ is —CH₃ then R⁴ or R⁷ is not —NR¹¹R¹².

In some embodiments, provided is a compound of Formula (II-A) to (II-X):

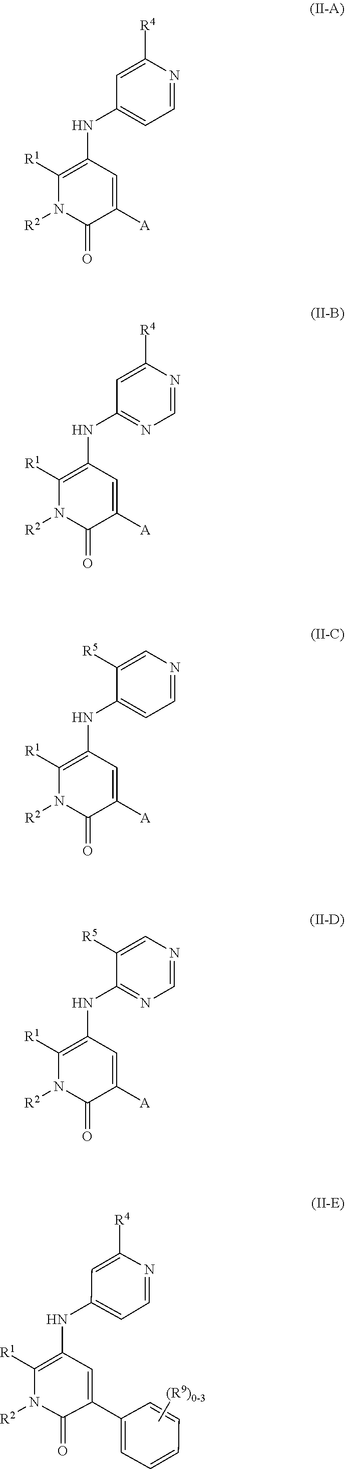

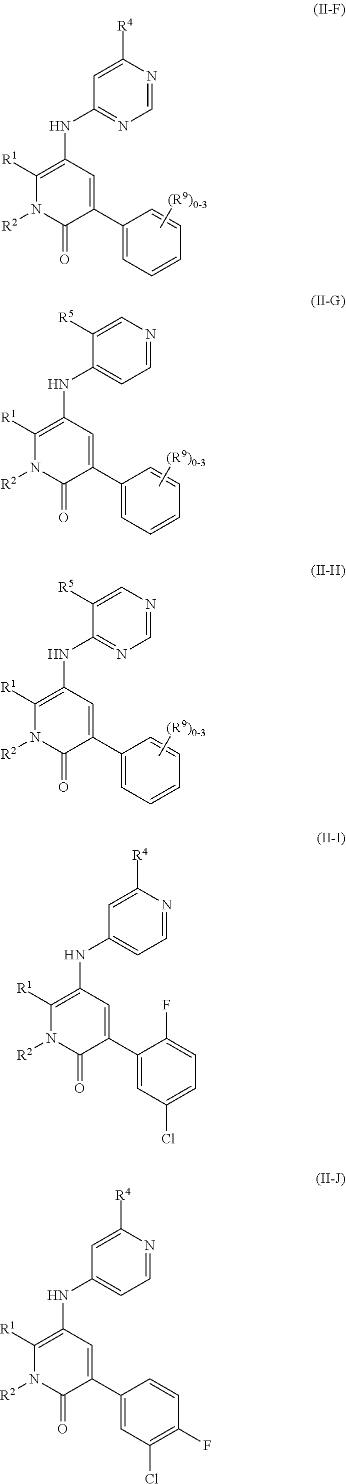

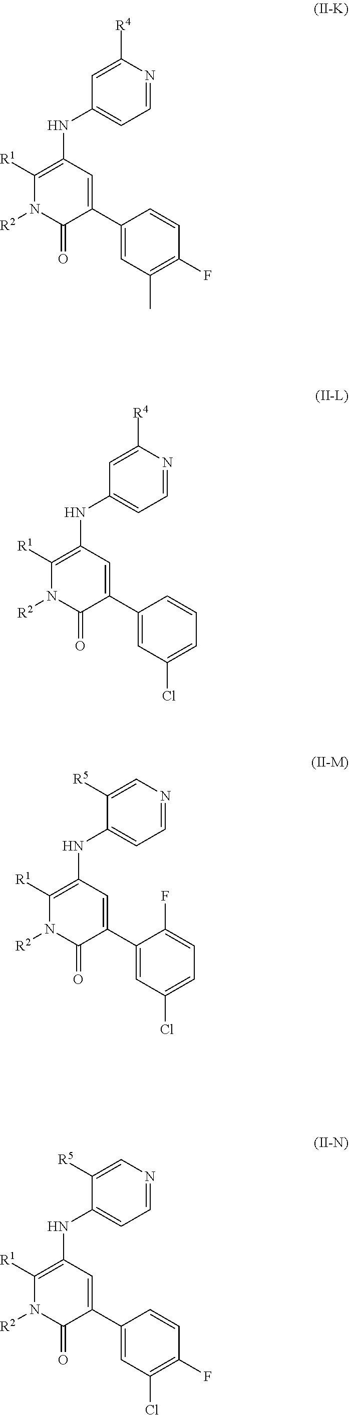

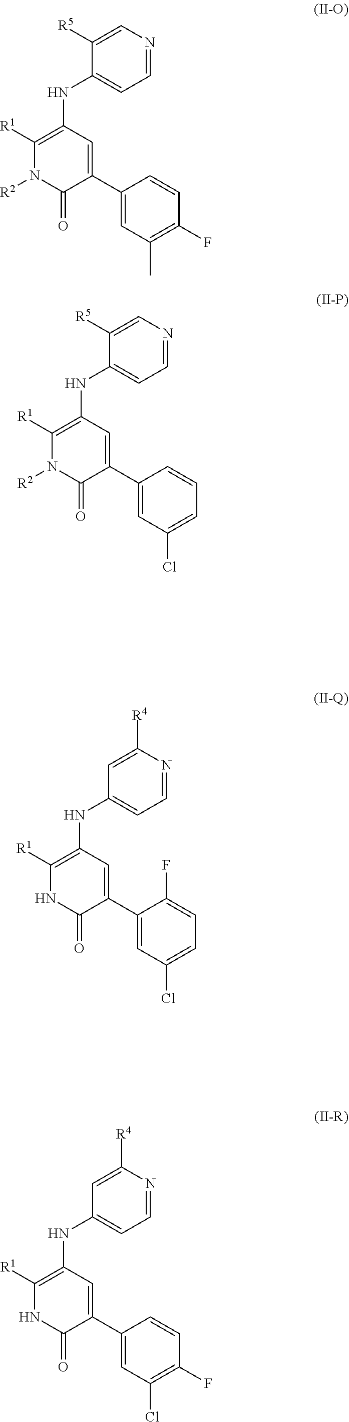

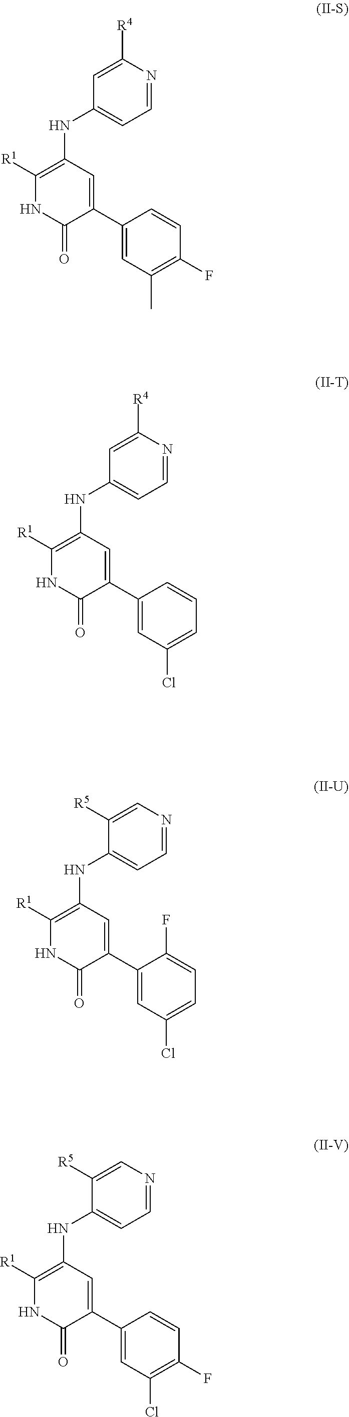

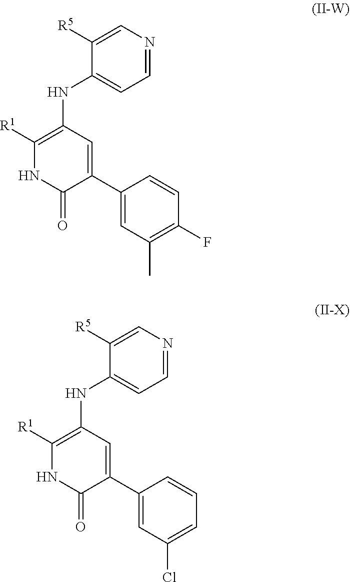

wherein A, R¹, R², R⁴, R⁵ and R⁹ are as defined for formula (I).

In some embodiments, provided is a compound of Formula (III):

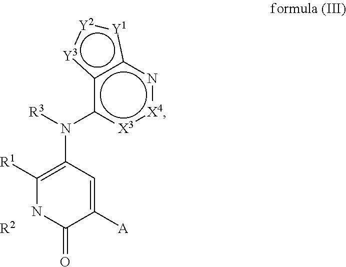

or a tautomer, salt, polymorph, solvate or stereoisomer thereof, wherein:

X³ is CH, N or CR⁶;

X⁴ is CH, N or CR⁷;

Y¹ and Y³ are independently N, NH, NR⁸, CH or CR⁸, S, O;

Y² is N, CH or CR⁸;

A, R¹, R², R³, R⁶, R⁷ and R⁸ are as defined herein.

provided that when R¹ is hydrogen then R⁸ is other than 6-membered heterocyclyl.

In some embodiments, provided is a compound of Formula (III-A) to (III-P):

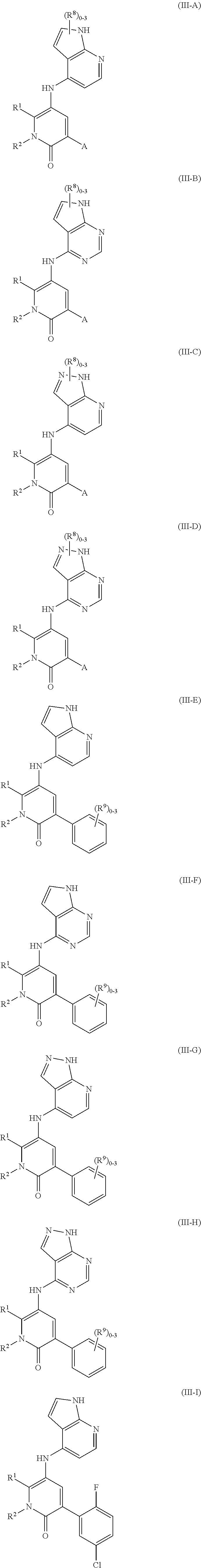

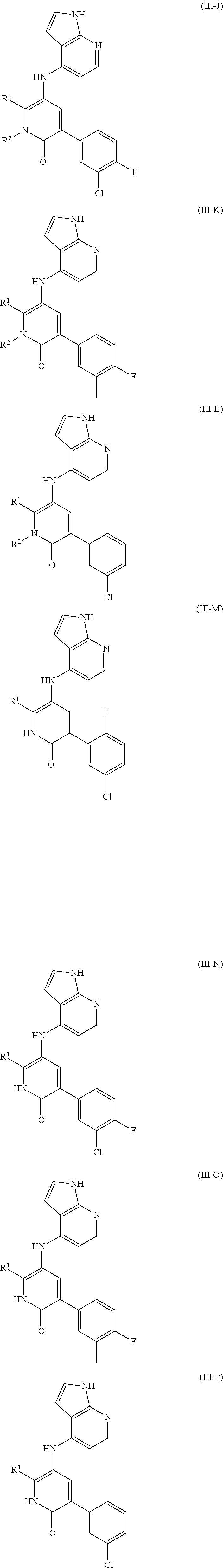

wherein A, R¹, R², R⁸ and R⁹ are as defined for formula (I).

In some embodiments, provided is a compound of formula (IV):

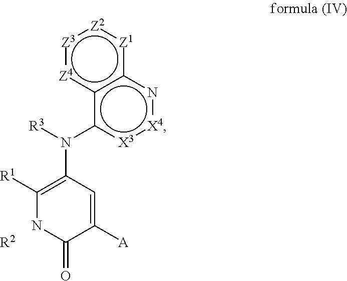

or a tautomer, salt, polymorph, solvate or stereoisomer thereof, wherein:

X³ is N, CH or CR⁶;

X⁴ is N, CH or CR⁷;

Z¹, Z², Z³ and Z⁴ are independently N, CH or CR⁸;

A, R¹, R², R³, R⁶, R⁷ and R⁸ are as defined herein.

In some embodiments, provided is a compound of Formula (IV-A) to (IV-P):

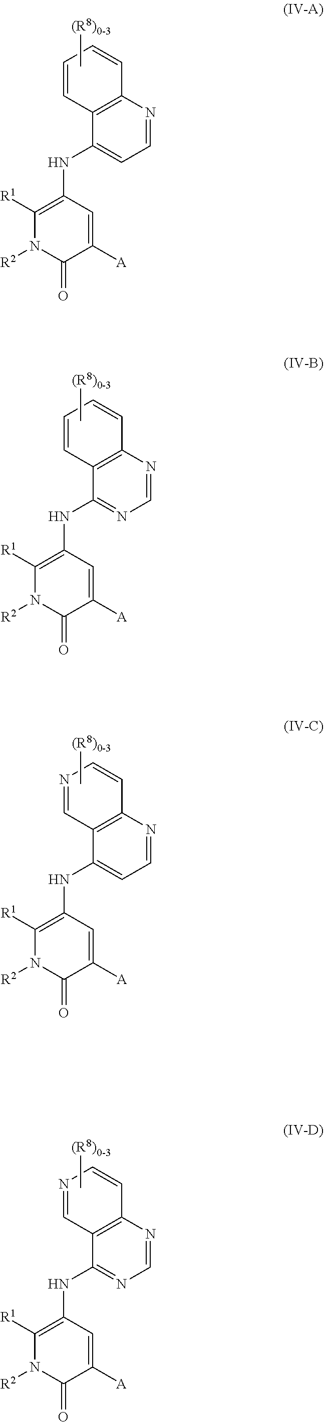

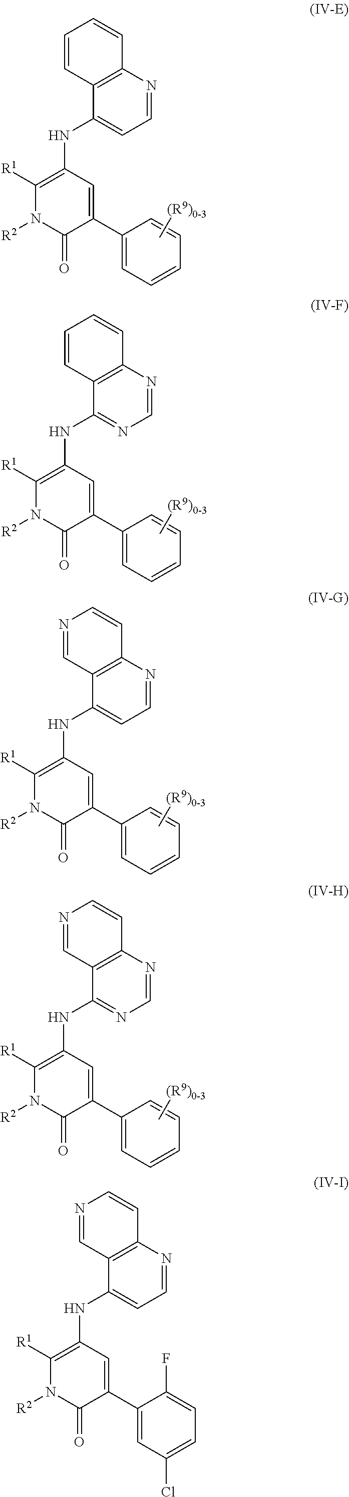

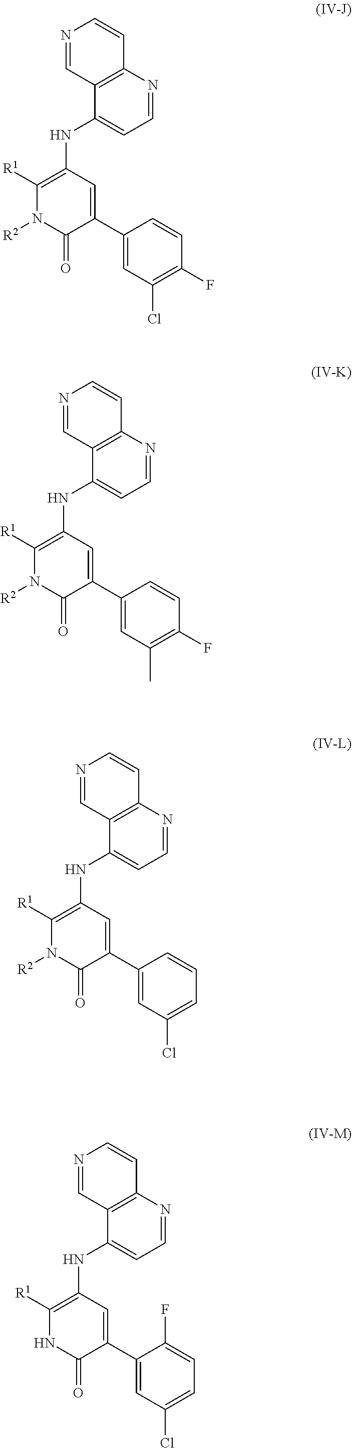

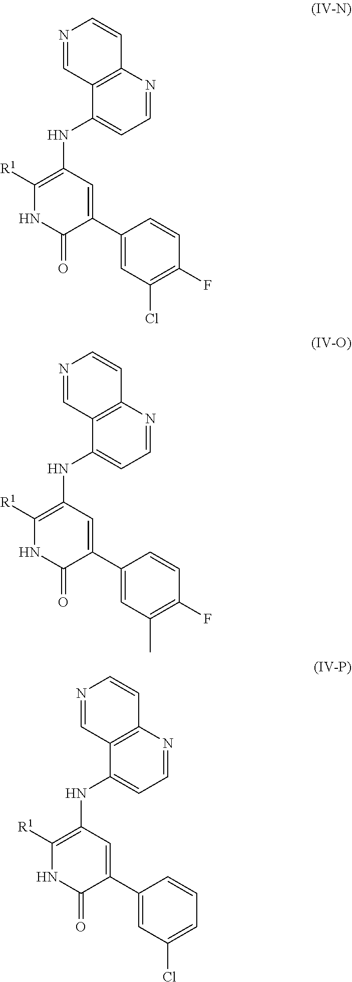

wherein A, R¹, R², R⁸ and R⁹ are as defined for formula (I).

In some embodiments, provided is a compound of formula (V):

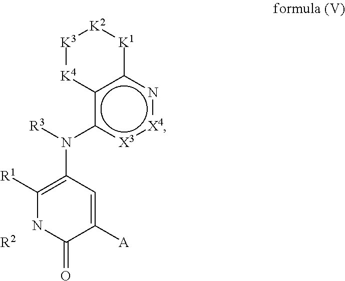

or a tautomer, salt, polymorph, solvate or stereoisomer thereof, wherein:

X³ is N, CH or CR⁶;

X⁴ is N, CH or CR⁷;

K¹ is S, O, NH, NR⁸, CH₂, CHR⁸, C═O or CR⁸R^{8′};

K² is NH, NR⁸, CH₂, CHR⁸, C═O or CR⁸R^{8′};

K³ is NH, NR⁸, CH₂, CHR⁸, C═O or CR⁸R^{8′} or absent;

K⁴ is S, O, NH, NR⁸, CH₂, CHR⁸, C═O or CR⁸R^{8′};
provided that:
i) no more than two of K¹, K², K³ and K⁴ are NH or NR⁸;
ii) if K³ is absent, then at least one of K¹ and K⁴ is not O or S;
iii) no more than two of K¹, K², K³ and K⁴ is C═O;

R⁸ and R^{8′} are independently hydrogen, halogen, oxo, —CN, C₁-C₆ alkyl, C₁-C₆ cycloalkyl, C₁-C₃ alkoxy, C₁-C₃ haloalkoxy, C₁-C₃ haloalkyl, 3- to 6-membered heterocyclyl, —NR¹¹R¹², —C(O)NR¹²R¹³, —NR¹¹C(O)R¹², —C(O)OR¹¹, —OR¹¹, —(C₁-C₃ alkylene)NR¹¹R¹² or —(C₁-C₃ alkylene)3- to 6-membered heterocyclyl, each of which is optionally substituted by C₁-C₆ alkyl, halogen, oxo, —CN, —OR¹⁵ or —NR¹⁵R¹⁶;

A, R¹, R², R³, R¹¹, R¹², R¹³, R¹⁵ and R¹⁶ are as defined herein.

In some embodiments, provided is a compound of Formula (V-A) to (V-L):

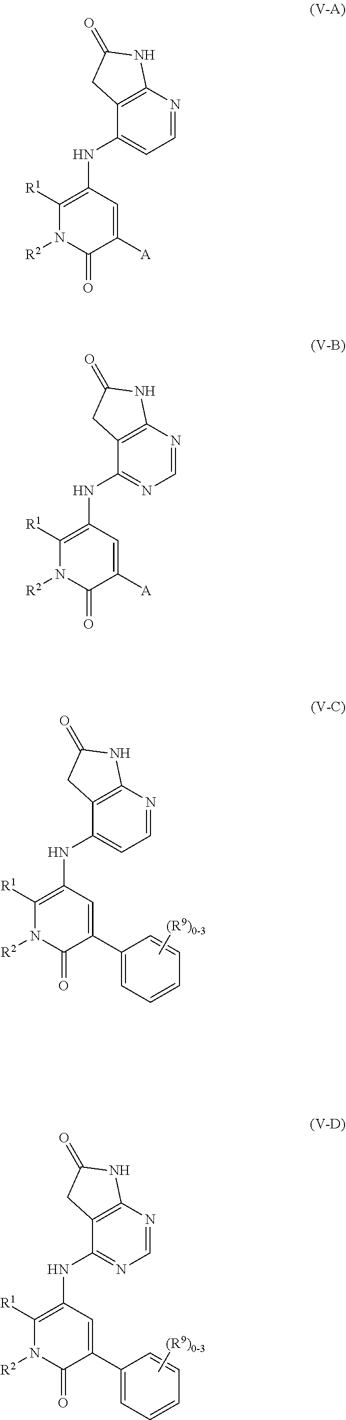

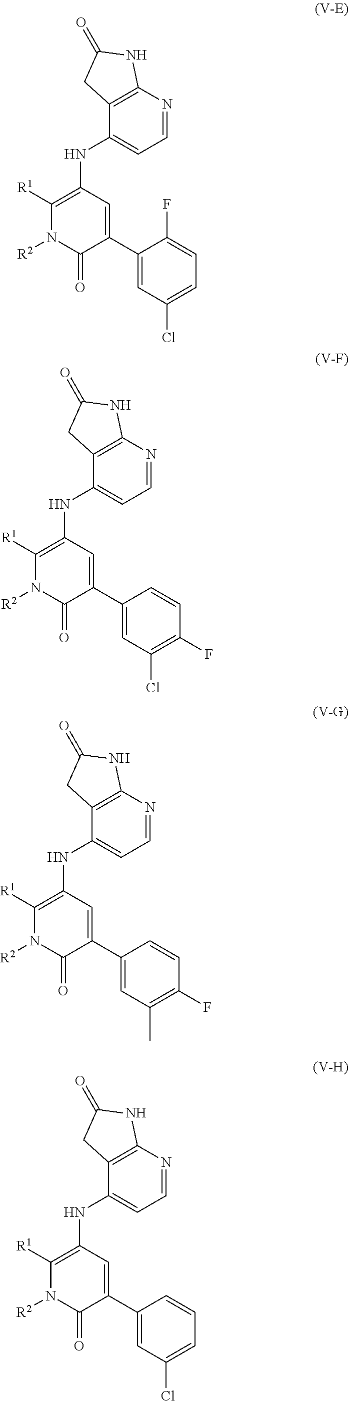

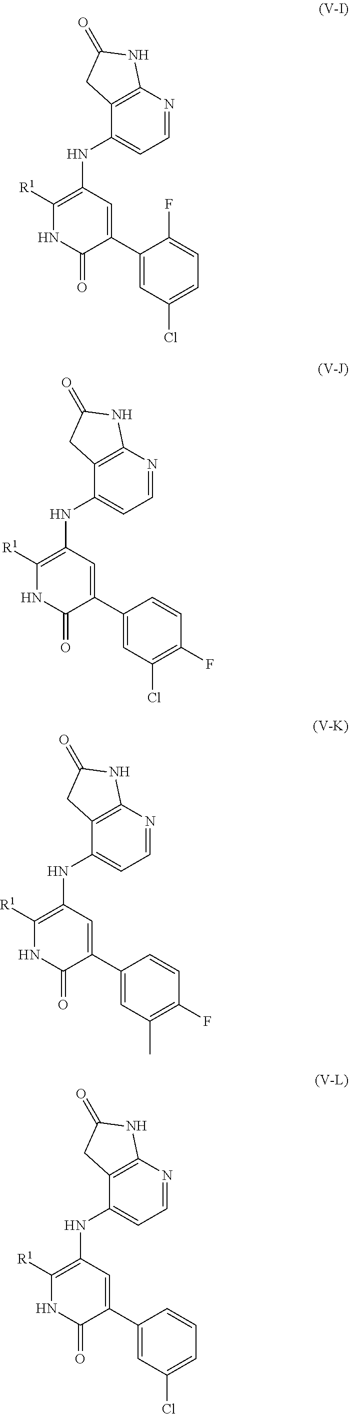

wherein A, R¹, R² and R⁹ are as defined for formula (I).

In some embodiments of a compound of formula (I), R² is hydrogen, R¹ is hydrogen, halogen, —CN, C₁-C₆ alkyl, C₂-C₆ alkenyl, C₂-C₆ alkynyl, C₃-C₆ cycloalkyl, 3- to 6-membered heterocyclyl, 5- to 6-membered heteroaryl, C₁-C₃ alkoxy, C₁-C₃ haloalkoxy, C₁-C₃ haloalkyl, —OR¹¹, —NR¹¹ R¹², —C(O)NR¹² R¹³, —C(O)R¹¹, —S(O)₂R¹¹, wherein each R¹ is optionally substituted with R¹⁰. In some embodiments of a compound of formula (I), R² is hydrogen, R¹ is hydrogen, halogen, —CN, C₁-C₆ alkyl, C₃-C₆ cycloalkyl, 3- to 6-membered heterocyclyl, C₁-C₃ haloalkoxy, C₁-C₃ haloalkyl, —OR¹¹, —NR¹¹ R¹², —C(O) NR¹² R¹³, —C(O) R¹¹, —S(O)₂R¹¹, wherein each R¹ is optionally substituted with R¹⁰. In some embodiments of a compound of formula (I), R² is hydrogen, R¹ is hydrogen, isopropyl, or —N(CH₃)₂. In some embodiments of a compound of formula (I), R² is hydrogen, R¹ is ethyl, isopropyl, cyclopropyl, —N(CH₃)₂ or —OCH₃.

In some embodiments of a compound of formula (I), R² is methyl, R¹ is hydrogen, halogen, —CN, C₁-C₆ alkyl, C₂-C₆ alkenyl, C₂-C₆ alkynyl, C₃-C₆ cycloalkyl, 3- to 6-membered heterocyclyl, 5- to 6-membered heteroaryl, C₁-C₃ alkoxy, C₁-C₃ haloalkoxy, C₁-C₃ haloalkyl, —OR¹¹, —NR¹¹ R¹², —C(O) NR¹² R¹³, —C(O) R¹¹, —S(O)₂R¹¹, wherein each R¹ is optionally substituted with R¹⁰. In some embodiments of a compound of formula (I), R² is methyl, R¹ is hydrogen, halogen, —CN, C₁-C₆ alkyl, C₃-C₆ cycloalkyl, 3- to 6-membered heterocyclyl, C₁₋C₃ haloalkoxy, C₁-C₃ haloalkyl, —OR¹¹, —NR¹¹ R¹², —C(O) NR¹² R¹³, —C(O) R¹¹, —S(O)₂R¹¹, wherein each R¹ is optionally substituted with R¹⁰. In some embodiments of a compound of formula (I), R² is methyl, R¹ is hydrogen, isopropyl, or —N(CH₃)₂.

In some embodiments of a compound of formula (I), R² is hydrogen, R¹ is hydrogen, ethyl, isopropyl, cyclopropyl, —OCH₃ or —N(CH₃)₂, A is C₆ aryl, 5- to 6-membered heteroaryl, C₃-C₆ cycloalkyl or 5- to 6-membered heterocyclyl, wherein A is optionally substituted with 0 to 3 R⁹ groups which may be same or different. In some embodiments of a compound of formula (I), R² is hydrogen, R¹ is hydrogen, ethyl, isopropyl, cyclopropyl, —OCH₃ or —N(CH₃)₂, A is phenyl, pyridyl, pyrimidinyl, furanyl, oxazolyl, thiophenyl, pyrazolyl, isoxazolyl, 1,3,4-oxadiazolyl, imidazolyl, thiazolyl, isothiazolyl, and 1,3,4-thiadiazolyl, each of which is optionally substituted with 0 to 3 R³ groups which may be same or different. In some embodiments of a compound of formula (I), R² is hydrogen, R¹ is hydrogen, ethyl, isopropyl, cyclopropyl, —OCH₃ or —N(CH₃)₂, A is phenyl or pyridyl, each of which is optionally substituted with 0 to 3 R⁹ groups which may be same or different. In some embodiments of a compound of formula (I), R² is hydrogen, R¹ is hydrogen, ethyl, isopropyl, cyclopropyl, —OCH₃ or —N(CH₃)₂, A is phenyl substituted with 0 to 3 halogen which may be same or different. In some embodiments of a compound of formula (I), R² is hydrogen, R¹ is hydrogen, ethyl, isopropyl, cyclopropyl, —OCH₃ or —N(CH₃)₂, A is phenyl substituted with C₁, F or both. In some embodiments of a compound of formula (I), R² is hydrogen, R¹ is hydrogen, ethyl, isopropyl, cyclopropyl, —OCH₃ or —N(CH₃)₂, A is pyridyl substituted with methyl, F or CF₃.

In some embodiments of a compound of formula (I), R² is methyl, R¹ is hydrogen, isopropyl, or —N(CH₃)₂, A is C₆ aryl, 5- to 6-membered heteroaryl, C₃-C₆ cycloalkyl or 5- to 6-membered heterocyclyl, wherein A is optionally substituted with 0 to 3 R⁹ groups which may be same or different. In some embodiments of a compound of formula (I), R² is methyl, R¹ is hydrogen, isopropyl, or —N(CH₃)₂, A is phenyl, pyridyl, pyrimidinyl, furanyl, oxazolyl, thiophenyl, pyrazolyl, isoxazolyl, 1,3,4-oxadiazolyl, imidazolyl, thiazolyl, isothiazolyl, and 1,3,4-thiadiazolyl, each of which is optionally substituted with 0 to 3 R⁹ groups which may be same or different. In some embodiments of a compound of formula (I), R² is methyl, R¹ is hydrogen, isopropyl, or —N(CH₃)₂, A is phenyl or pyridyl, each of which is optionally substituted with 0 to 3 R⁹ groups which may be same or different. In some embodiments of a compound of formula (I), R² is methyl, R¹ is hydrogen, isopropyl, or —N(CH₃)₂, A is phenyl substituted with Cl, F or both. In some embodiments of a compound of formula (I), R² is methyl, R¹ is hydrogen, isopropyl, or —N(CH₃)₂, A is pyridyl substituted with methyl.

In some embodiments of a compound of formula (I), R² is hydrogen, R¹ is hydrogen, ethyl, isopropyl, cyclopropyl, —OCH₃ or —N(CH₃)₂, A is phenyl substituted with Cl, F or both, Cy is optionally substituted pyridyl or pyrimidyl. In some embodiments of a compound of formula (I), R² is hydrogen, R¹ is hydrogen, ethyl, isopropyl, cyclopropyl, —OCH₃ or —N(CH₃)₂, A is phenyl substituted with C₁, F or both, Cy is optionally substituted pyridyl. In some embodiments of a compound of formula (I), R² is hydrogen, R¹ is hydrogen, ethyl, isopropyl, cyclopropyl, —OCH₃ or —N(CH₃)₂, A is phenyl substituted with Cl, F or both, Cy is optionally substituted pyrrolopyridinyl, pyrrolopyridazinyl, pyrrolopyrimidinyl, pyrazolopyridinyl, pyrazolopyrimidinyl, dihydropyrrolopyridinyl, dihydrocyclopentapyridinyl, imidazopyridinyl, purinyl, quinolinyl, 1,8-naphthyridinyl, 1,7-naphthyridinyl, 1,6-naphthyridinyl, 1,5-naphthyridinyl, quinazolinyl, pyridopyrimidinyl, cinnolinyl or pyridopyridazinyl. In some embodiments of a compound of formula (I), R² is hydrogen, R¹ is hydrogen, ethyl, isopropyl, cyclopropyl, —OCH₃ or —N(CH₃)₂, A is phenyl substituted with Cl, F or both, Cy is optionally substituted pyrrolopyridinyl or quinolinyl. In some embodiments of a compound of formula (I), R² is hydrogen, R¹ is hydrogen, ethyl, isopropyl, cyclopropyl, —OCH₃ or —N(CH₃)₂, A is phenyl substituted with Cl, F or both, Cy is optionally substituted pyrrolopyridinyl.

In some embodiments of a compound of formula (I), R² is methyl, R¹ is hydrogen, isopropyl, or —N(CH₃)₂, A is phenyl substituted with Cl, F or both, Cy is optionally substituted pyridyl or pyrimidyl. In some embodiments of a compound of formula (I), R² is hydrogen, R¹ is methyl, isopropyl, or —N(CH₃)₂, A is phenyl substituted with Cl, F or both, Cy is optionally substituted pyridyl. In some embodiments of a compound of formula (I), R² is methyl, R¹ is hydrogen, isopropyl, or —N(CH₃)₂, A is phenyl substituted with C₁, F or both, Cy is optionally substituted pyrrolopyridinyl, pyrrolopyridazinyl, pyrrolopyrimidinyl, pyrazolopyridinyl, pyrazolopyrimidinyl, dihydropyrrolopyridinyl, dihydrocyclopentapyridinyl, imidazopyridinyl, purinyl, quinolinyl, 1,8-naphthyridinyl, 1,7-naphthyridinyl, 1,6-naphthyridinyl, 1,5-naphthyridinyl, quinazolinyl, pyridopyrimidinyl, cinnolinyl or pyridopyridazinyl. In some embodiments of a compound of formula (I), R² is methyl, R¹ is hydrogen, isopropyl, or —N(CH₃)₂, A is phenyl substituted with C₁, F or both, Cy is optionally substituted pyrrolopyridinyl or quinolinyl. In some embodiments of a compound of formula (I), R² is methyl, R¹ is hydrogen, isopropyl, or —N(CH₃)₂, A is phenyl substituted with Cl, F or both, Cy is optionally substituted pyrrolopyridinyl.

In some embodiments of a compound of formula (I), R² is hydrogen, R¹ is hydrogen, ethyl, isopropyl, or cyclopropyl, —OCH₃—N(CH₃)₂, A is pyridyl substituted with methyl, Cy is optionally substituted pyridyl or pyrimidyl. In some embodiments of a compound of formula (I), R² is hydrogen, R¹ is hydrogen, ethyl, isopropyl, or cyclopropyl, —OCH₃—N(CH₃)₂, A is pyridyl substituted with methyl, Cy is optionally substituted pyridyl. In some embodiments of a compound of formula (I), R² is hydrogen, R¹ is hydrogen, ethyl, isopropyl, cyclopropyl, —OCH₃ or —N(CH₃)₂, A is pyridyl substituted with methyl, Cy is optionally substituted pyrrolopyridinyl, pyrrolopyridazinyl, pyrrolopyrimidinyl, pyrazolopyridinyl, pyrazolopyrimidinyl, dihydropyrrolopyridinyl, dihydrocyclopentapyridinyl, imidazopyridinyl, purinyl, quinolinyl, 1,8-naphthyridinyl, 1,7-naphthyridinyl, 1,6-naphthyridinyl, 1,5-naphthyridinyl, quinazolinyl, pyridopyrimidinyl, cinnolinyl or pyridopyridazinyl. In some embodiments of a compound of formula (I), R² is hydrogen, R¹ is hydrogen, ethyl, isopropyl, cyclopropyl, —OCH₃ or —N(CH₃)₂, A is pyridyl substituted with methyl, Cy is optionally substituted pyrrolopyridinyl or quinolinyl. In some embodiments of a compound of formula (I), R² is hydrogen, R¹ is hydrogen, ethyl, isopropyl, cyclopropyl, —OCH₃ or —N(CH₃)₂, A is pyridyl substituted with methyl, Cy is optionally substituted pyrrolopyridinyl.

In some embodiments of a compound of formula (I), R² is methyl, R¹ is hydrogen, isopropyl, or —N(CH₃)₂, A is pyridyl substituted with methyl, Cy is optionally substituted pyridyl or pyrimidyl. In some embodiments of a compound of formula (I), R² is methyl, R¹ is hydrogen, isopropyl, or —N(CH₃)₂, A is pyridyl substituted methyl, Cy is optionally substituted pyrrolopyridinyl, pyrrolopyridazinyl, pyrrolopyrimidinyl, pyrazolopyridinyl, pyrazolopyrimidinyl, dihydropyrrolopyridinyl, dihydrocyclopentapyridinyl, imidazopyridinyl, purinyl, quinolinyl, 1,8-naphthyridinyl, 1,7-naphthyridinyl, 1,6-naphthyridinyl, 1,5-naphthyridinyl, quinazolinyl, pyridopyrimidinyl, cinnolinyl or pyridopyridazinyl. In some embodiments of a compound of formula (I), R² is methyl, R¹ is hydrogen, isopropyl, or —N(CH₃)₂, A is pyridyl substituted with methyl, Cy is optionally substituted pyrrolopyridinyl or quinolinyl. In some embodiments of a compound of formula (I), R² is methyl, R¹ is hydrogen, isopropyl, or —N(CH₃)₂, A is pyridyl substituted with methyl, Cy is optionally substituted pyrrolopyridinyl.

Particular compounds described below are not intended to be limiting; rather, these embodiments and variations are intended to provide examples of compounds within the scope of Formula (I), (II), (II-A) to (II-X), (III), (III-A) to (III-P), (IV), (IV-A) to (IV-P), (V) or (V-A) to (V-L).

Also provided are salts of compounds referred to herein, such as pharmaceutically acceptable salts. The invention also includes any or all of the stereochemical forms, including any enantiomeric or diastereomeric forms, and any tautomers or other forms of the compounds described.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. Unless otherwise stated, “substantially pure” intends a composition that contains no more than 35% impurity, wherein the impurity denotes a compound other than the compound comprising the majority of the composition or a salt thereof. In some embodiments, a composition of substantially pure compound or a salt thereof is provided wherein the composition contains no more than 25%, 20%, 15%, 10%, or 5% impurity. In some embodiments, a composition of substantially pure compound or a salt thereof is provided wherein the composition contains or no more than 3%, 2%, 1% or 0.5% impurity.

Representative compounds of the present invention (collectively, a compound of formula (I), (II), (II-A) to (II-X), (III), (III-A) to (III-P), (IV), (IV-A) to (IV-P), (V) or (V-A) to (V-L)) are listed in table 1 and table 2. It is understood that individual enantiomers and diastereomers are included in the generic compound structures shown in table 1 and table 2. Specific synthetic methods for preparing compounds of Table 1 are provided example herein.

TABLE 1
Compounds

| | |
|---|---|
| 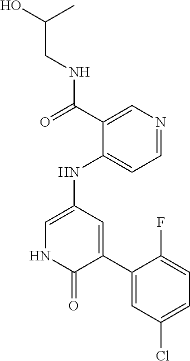 [SMILES: CC(O)CNC(=O)c1cnccc1Nc1c[nH]c(=O)c(-c2cc(Cl)ccc2F)c1] | 1.1 |
| 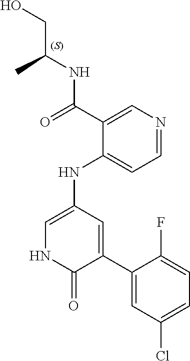 | 1.2 |
| 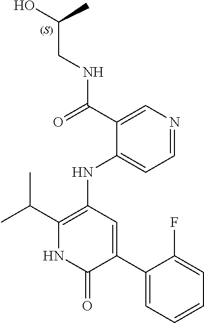 | 1.3 |
| 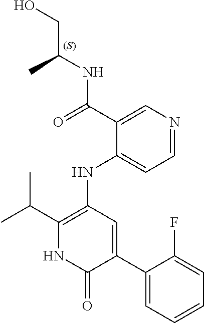 | 1.4 |
| 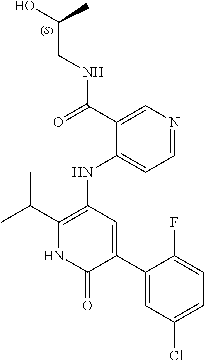 | 1.5 |
| 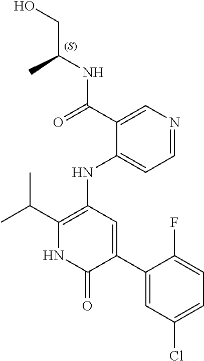 | 1.6 |
| 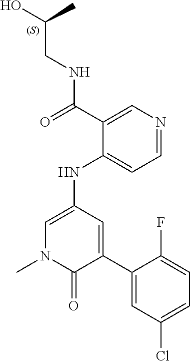 | 1.7 |
| 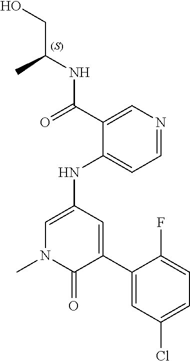 | 1.8 |
| 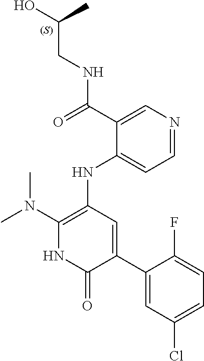 | 1.9 |
| 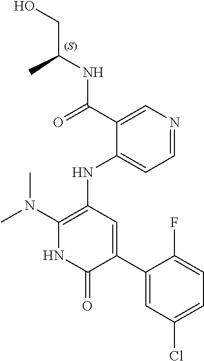 | 1.10 |
| 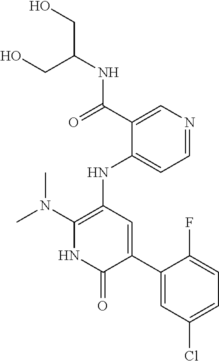 [SMILES: CN(C)c1[nH]c(=O)c(-c2cc(Cl)ccc2F)cc1Nc1ccncc1C(=O)NC(CO)CO] | 1.11 |
| 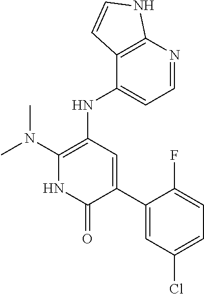 [SMILES: CN(C)c1[nH]c(=O)c(-c2cc(Cl)ccc2F)cc1Nc1ccnc2[nH]ccc12] | 1.12 |
| 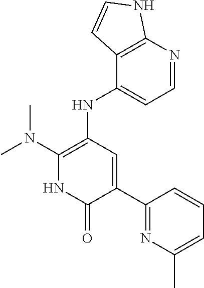 [SMILES: Cc1cccc(-c2cc(Nc3ccnc4[nH]ccc34)c(N(C)C)[nH]c2=O)n1] | 1.13 |
| 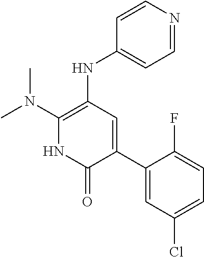 [SMILES: CN(C)c1[nH]c(=O)c(-c2cc(Cl)ccc2F)cc1Nc1ccncc1] | 1.14 |
| 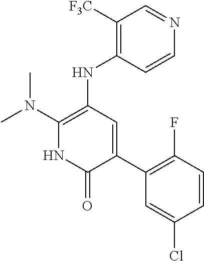 [SMILES: CN(C)c1[nH]c(=O)c(-c2cc(Cl)ccc2F)cc1Nc1ccncc1C(F)(F)F] | 1.15 |
| 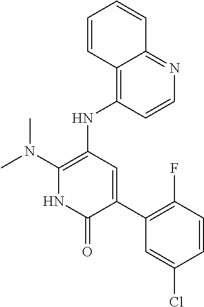 [SMILES: CN(C)c1[nH]c(=O)c(-c2cc(Cl)ccc2F)cc1Nc1ccnc2ccccc12] | 1.16 |
| 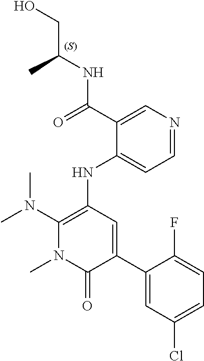 | 1.17 |
| 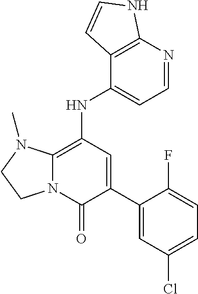 [SMILES: CN1CCn2c1c(Nc1ccnc3[nH]ccc13)cc(-c1cc(Cl)ccc1F)c2=O] | 1.18 |
| 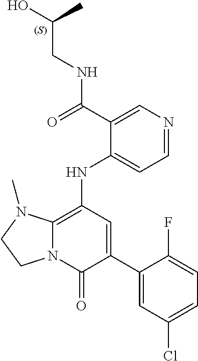 | 1.19 |
| 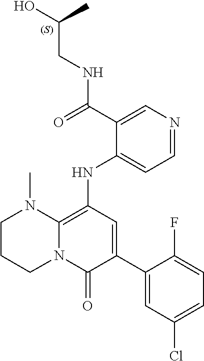 | 1.20 |
| 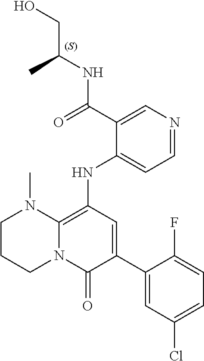 | 1.21 |
| 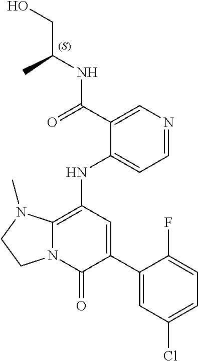 | 1.22 |
| 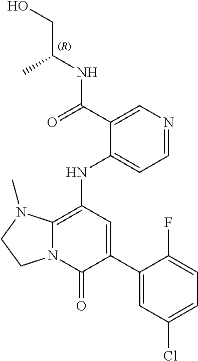 | 1.23 |
| 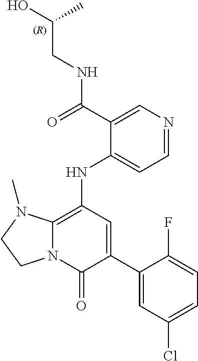 | 1.24 |
| 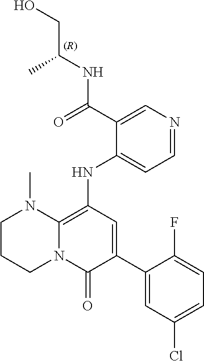 | 1.25 |
| 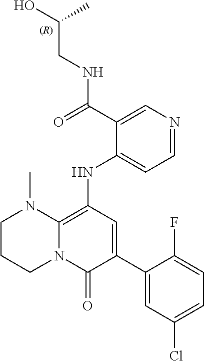 | 1.26 |
| 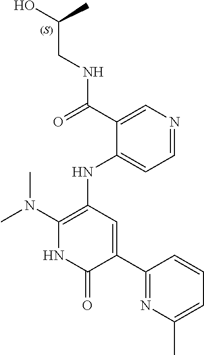 | 1.27 |
| 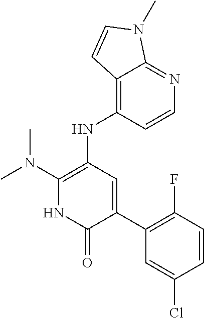 [SMILES: CN(C)c1[nH]c(=O)c(-c2cc(Cl)ccc2F)cc1Nc1ccnc2c1ccn2C] | 1.28 |
| 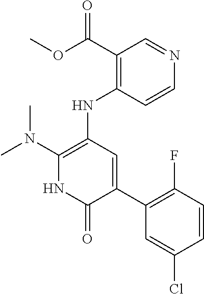 [SMILES: COC(=O)c1cnccc1Nc1cc(-c2cc(Cl)ccc2F)c(=O)[nH]c1N(C)C] | 1.29 |
| 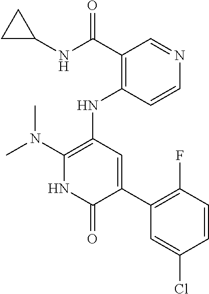 [SMILES: CN(C)c1[nH]c(=O)c(-c2cc(Cl)ccc2F)cc1Nc1ccncc1C(=O)NC1CC1] | 1.30 |
| 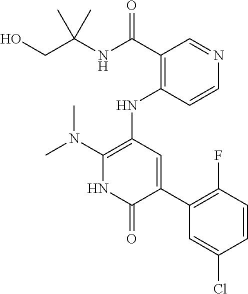 [SMILES: CN(C)c1[nH]c(=O)c(-c2cc(Cl)ccc2F)cc1Nc1ccncc1C(=O)NC(C)(C)CO] | 1.31 |
| 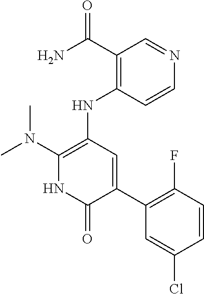 [SMILES: CN(C)c1[nH]c(=O)c(-c2cc(Cl)ccc2F)cc1Nc1ccncc1C(N)=O] | 1.32 |
| 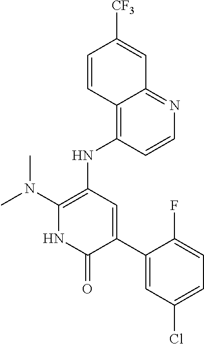 [SMILES: CN(C)c1[nH]c(=O)c(-c2cc(Cl)ccc2F)cc1Nc1ccnc2cc(C(F)(F)F)ccc12] | 1.33 |
| 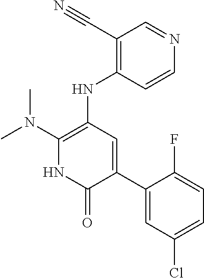 [SMILES: CN(C)c1[nH]c(=O)c(-c2cc(Cl)ccc2F)cc1Nc1ccncc1C#N] | 1.34 |
| 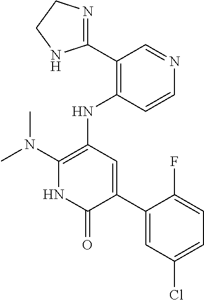 [SMILES: CN(C)c1[nH]c(=O)c(-c2cc(Cl)ccc2F)cc1Nc1ccncc1C1=NCCN1] | 1.35 |
| 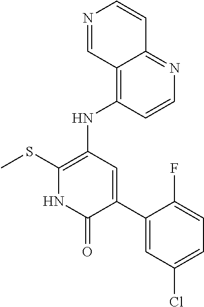 [SMILES: CSc1[nH]c(=O)c(-c2cc(Cl)ccc2F)cc1Nc1ccnc2ccncc12] | 1.36 |
| 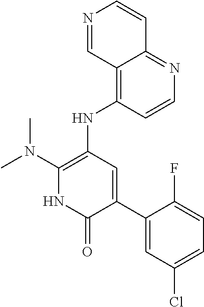 [SMILES: CN(C)c1[nH]c(=O)c(-c2cc(Cl)ccc2F)cc1Nc1ccnc2ccncc12] | 1.37 |
| 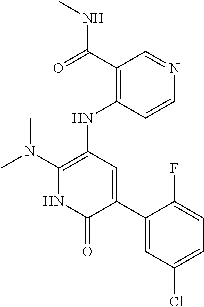 [SMILES: CNC(=O)c1cnccc1Nc1cc(-c2cc(Cl)ccc2F)c(=O)[nH]c1N(C)C] | 1.38 |
| 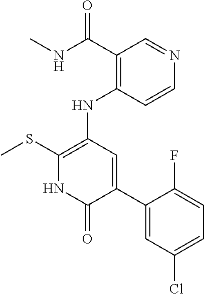 [SMILES: CNC(=O)c1cnccc1Nc1cc(-c2cc(Cl)ccc2F)c(=O)[nH]c1SC] | 1.39 |
| 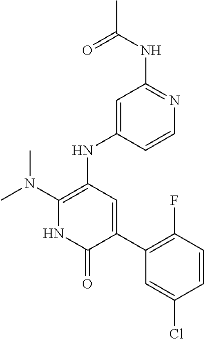 [SMILES: CC(=O)Nc1cc(Nc2cc(-c3cc(Cl)ccc3F)c(=O)[nH]c2N(C)C)ccn1] | 1.40 |
| 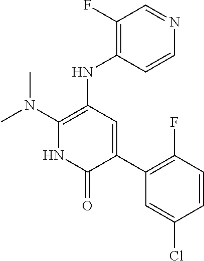 [SMILES: CN(C)c1[nH]c(=O)c(-c2cc(Cl)ccc2F)cc1Nc1ccncc1F] | 1.41 |
| 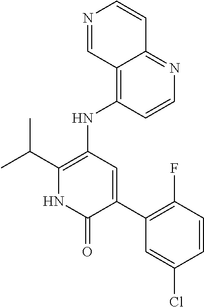 [SMILES: CC(C)c1[nH]c(=O)c(-c2cc(Cl)ccc2F)cc1Nc1ccnc2ccncc12] | 1.42 |
| 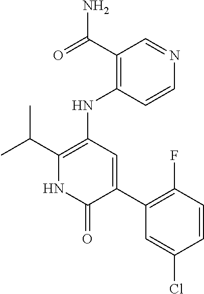 [SMILES: CC(C)c1[nH]c(=O)c(-c2cc(Cl)ccc2F)cc1Nc1ccncc1C(N)=O] | 1.43 |
| 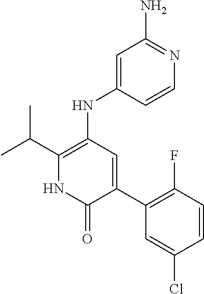 [SMILES: CC(C)c1[nH]c(=O)c(-c2cc(Cl)ccc2F)cc1Nc1ccnc(N)c1] | 1.44 |
| 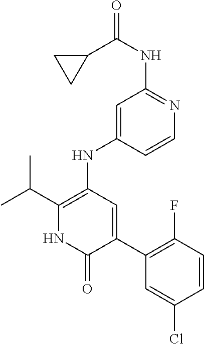 [SMILES: CC(C)c1[nH]c(=O)c(-c2cc(Cl)ccc2F)cc1Nc1ccnc(NC(=O)C2CC2)c1] | 1.45 |
| 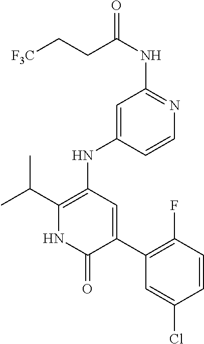 [SMILES: CC(C)c1[nH]c(=O)c(-c2cc(Cl)ccc2F)cc1Nc1ccnc(NC(=O)CCC(F)(F)F)c1] | 1.46 |
| 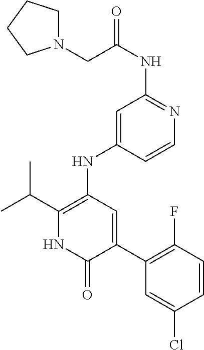 [SMILES: CC(C)c1[nH]c(=O)c(-c2cc(Cl)ccc2F)cc1Nc1ccnc(NC(=O)CN2CCCC2)c1] | 1.47 |
| 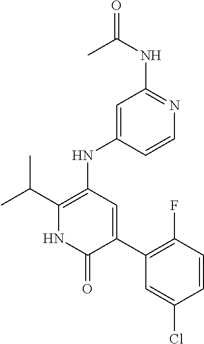 [SMILES: CC(=O)Nc1cc(Nc2cc(-c3cc(Cl)ccc3F)c(=O)[nH]c2C(C)C)ccn1] | 1.48 |
| 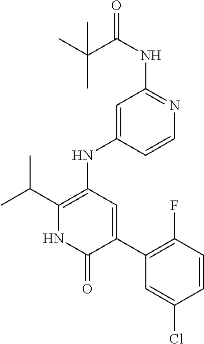 [SMILES: CC(C)c1[nH]c(=O)c(-c2cc(Cl)ccc2F)cc1Nc1ccnc(NC(=O)C(C)(C)C)c1] | 1.49 |
| 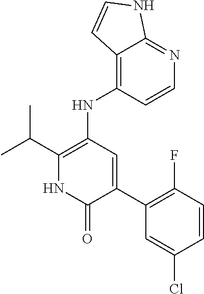 [SMILES: CC(C)c1[nH]c(=O)c(-c2cc(Cl)ccc2F)cc1Nc1ccnc2[nH]ccc12] | 1.50 |
| 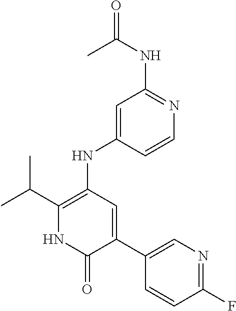 [SMILES: CC(=O)Nc1cc(Nc2cc(-c3ccc(F)nc3)c(=O)[nH]c2C(C)C)ccn1] | 1.51 |
| 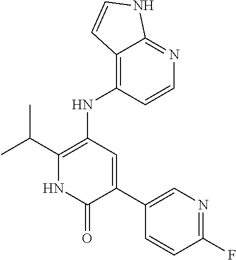 [SMILES: CC(C)c1[nH]c(=O)c(-c2ccc(F)nc2)cc1Nc1ccnc2[nH]ccc12] | 1.52 |
| 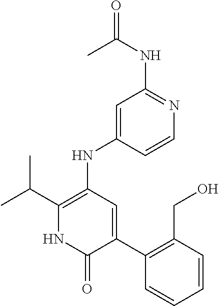 [SMILES: CC(=O)Nc1cc(Nc2cc(-c3ccccc3CO)c(=O)[nH]c2C(C)C)ccn1] | 1.53 |
| 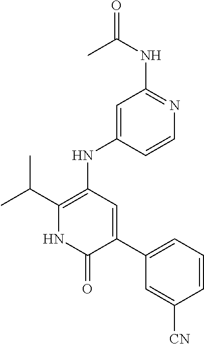 [SMILES: CC(=O)Nc1cc(Nc2cc(-c3cccc(C#N)c3)c(=O)[nH]c2C(C)C)ccn1] | 1.54 |
| 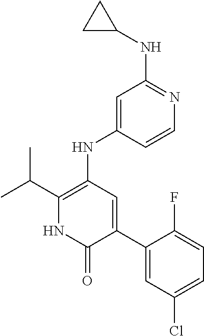 [SMILES: CC(C)c1[nH]c(=O)c(-c2cc(Cl)ccc2F)cc1Nc1ccnc(NC2CC2)c1] | 1.55 |
| 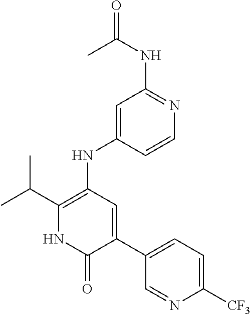 [SMILES: CC(=O)Nc1cc(Nc2cc(-c3ccc(C(F)(F)F)nc3)c(=O)[nH]c2C(C)C)ccn1] | 1.56 |
| 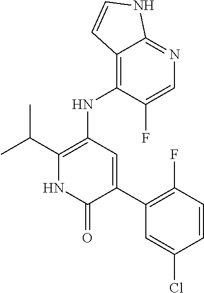 [SMILES: CC(C)c1[nH]c(=O)c(-c2cc(Cl)ccc2F)cc1Nc1c(F)cnc2[nH]ccc12] | 1.57 |
| 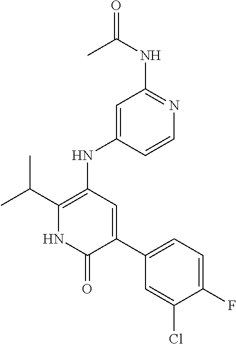 [SMILES: CC(=O)Nc1cc(Nc2cc(-c3ccc(F)c(Cl)c3)c(=O)[nH]c2C(C)C)ccn1] | 1.58 |
| 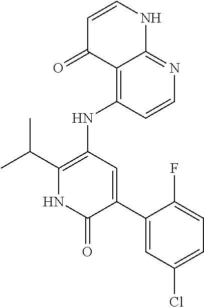 [SMILES: CC(C)c1[nH]c(=O)c(-c2cc(Cl)ccc2F)cc1Nc1ccnc2[nH]ccc(=O)c12] | 1.59 |
| 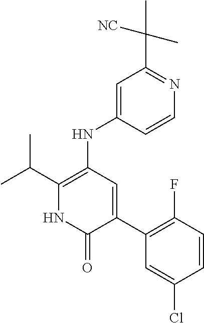 [SMILES: CC(C)c1[nH]c(=O)c(-c2cc(Cl)ccc2F)cc1Nc1ccnc(C(C)(C)C#N)c1] | 1.60 |
| 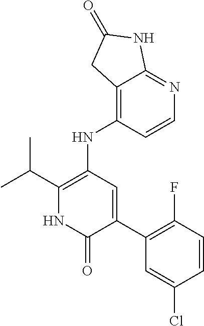 [SMILES: CC(C)c1[nH]c(=O)c(-c2cc(Cl)ccc2F)cc1Nc1ccnc2c1CC(=O)N2] | 1.61 |
| 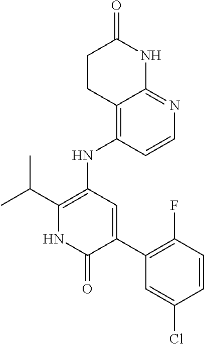 [SMILES: CC(C)c1[nH]c(=O)c(-c2cc(Cl)ccc2F)cc1Nc1ccnc2c1CCC(=O)N2] | 1.62 |
| 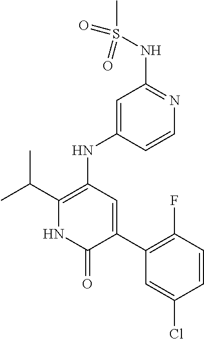 [SMILES: CC(C)c1[nH]c(=O)c(-c2cc(Cl)ccc2F)cc1Nc1ccnc(NS(C)(=O)=O)c1] | 1.63 |
| 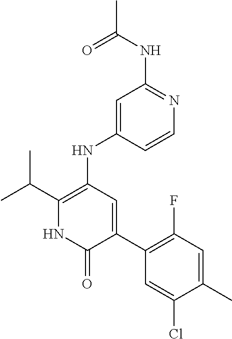 [SMILES: CC(=O)Nc1cc(Nc2cc(-c3cc(Cl)c(C)cc3F)c(=O)[nH]c2C(C)C)ccn1] | 1.64 |
| 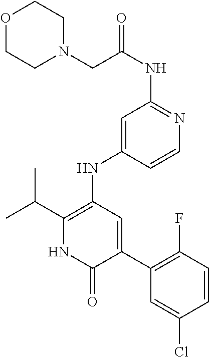 [SMILES: CC(C)c1[nH]c(=O)c(-c2cc(Cl)ccc2F)cc1Nc1ccnc(NC(=O)CN2CCOCC2)c1] | 1.65 |
| 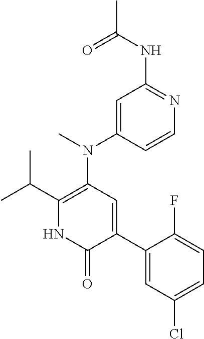 [SMILES: CC(=O)Nc1cc(N(C)c2cc(-c3cc(Cl)ccc3F)c(=O)[nH]c2C(C)C)ccn1] | 1.66 |
| 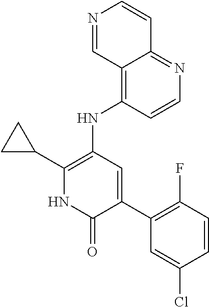 [SMILES: O=c1[nH]c(C2CC2)c(Nc2ccnc3ccncc23)cc1-c1cc(Cl)ccc1F] | 1.67 |
| 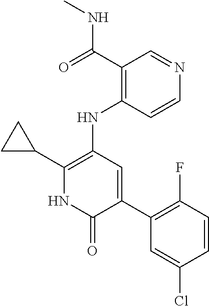 [SMILES: CNC(=O)c1cnccc1Nc1cc(-c2cc(Cl)ccc2F)c(=O)[nH]c1C1CC1] | 1.68 |
| 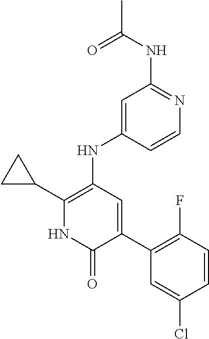 [SMILES: CC(=O)Nc1cc(Nc2cc(-c3cc(Cl)ccc3F)c(=O)[nH]c2C2CC2)ccn1] | 1.69 |
| 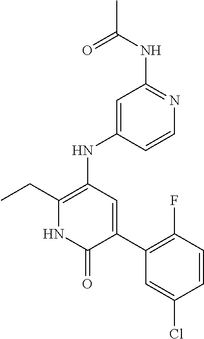 [SMILES: CCc1[nH]c(=O)c(-c2cc(Cl)ccc2F)cc1Nc1ccnc(NC(C)=O)c1] | 1.70 |
| 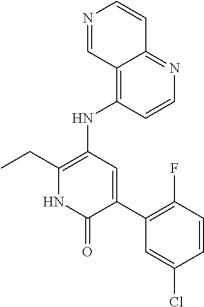 [SMILES: CCc1[nH]c(=O)c(-c2cc(Cl)ccc2F)cc1Nc1ccnc2ccncc12] | 1.71 |
| 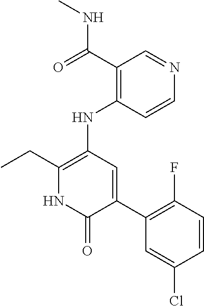 [SMILES: CCc1[nH]c(=O)c(-c2cc(Cl)ccc2F)cc1Nc1ccncc1C(=O)NC] | 1.72 |
| 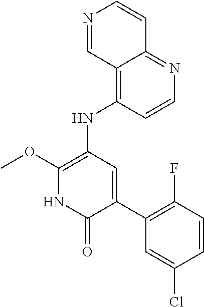 [SMILES: COc1[nH]c(=O)c(-c2cc(Cl)ccc2F)cc1Nc1ccnc2ccncc12] | 1.73 |
| 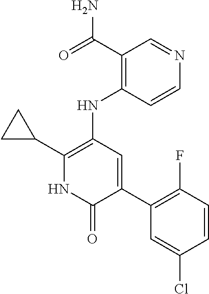 [SMILES: NC(=O)c1cnccc1Nc1cc(-c2cc(Cl)ccc2F)c(=O)[nH]c1C1CC1] | 1.74 |
| 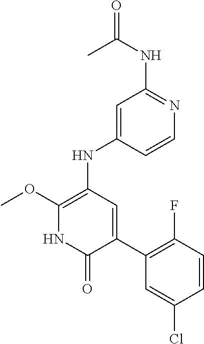 [SMILES: COc1[nH]c(=O)c(-c2cc(Cl)ccc2F)cc1Nc1ccnc(NC(C)=O)c1] | 1.75 |
| 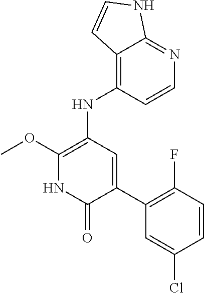 [SMILES: COc1[nH]c(=O)c(-c2cc(Cl)ccc2F)cc1Nc1ccnc2[nH]ccc12] | 1.76 |
| 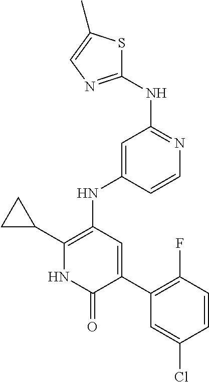 [SMILES: Cc1cnc(Nc2cc(Nc3cc(-c4cc(Cl)ccc4F)c(=O)[nH]c3C3CC3)ccn2)s1] | 1.77 |
| 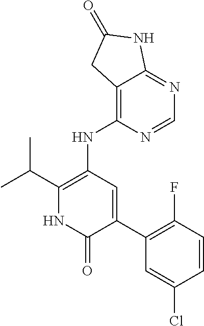 [SMILES: CC(C)c1[nH]c(=O)c(-c2cc(Cl)ccc2F)cc1Nc1ncnc2c1CC(=O)N2] | 1.78 |
| 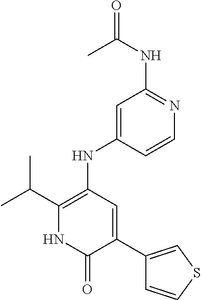 [SMILES: CC(=O)Nc1cc(Nc2cc(-c3ccsc3)c(=O)[nH]c2C(C)C)ccn1] | 1.79 |
| 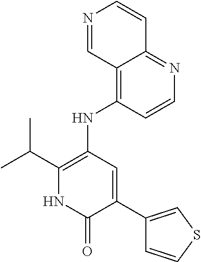 [SMILES: CC(C)c1[nH]c(=O)c(-c2ccsc2)cc1Nc1ccnc2ccncc12] | 1.80 |
| 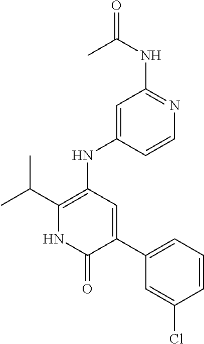 [SMILES: CC(=O)Nc1cc(Nc2cc(-c3cccc(Cl)c3)c(=O)[nH]c2C(C)C)ccn1] | 1.81 |
| 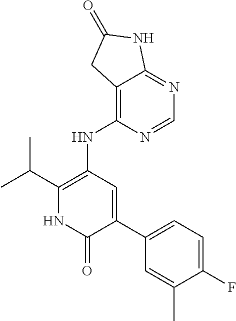 [SMILES: Cc1cc(-c2cc(Nc3ncnc4c3CC(=O)N4)c(C(C)C)[nH]c2=O)ccc1F] | 1.82 |
| 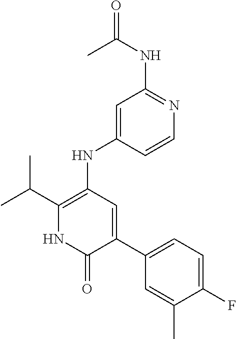 [SMILES: CC(=O)Nc1cc(Nc2cc(-c3ccc(F)c(C)c3)c(=O)[nH]c2C(C)C)ccn1] | 1.83 |
| 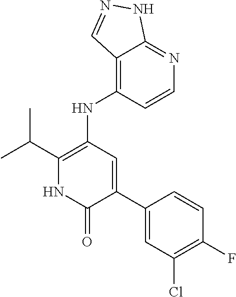 [SMILES: CC(C)c1[nH]c(=O)c(-c2ccc(F)c(Cl)c2)cc1Nc1ccnc2[nH]ncc12] | 1.84 |
| 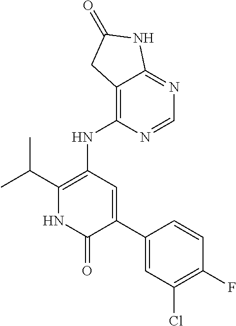 [SMILES: CC(C)c1[nH]c(=O)c(-c2ccc(F)c(Cl)c2)cc1Nc1ncnc2c1CC(=O)N2] | 1.85 |
| 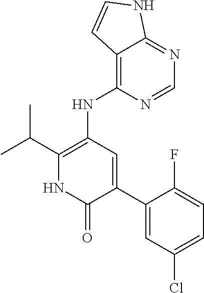 [SMILES: CC(C)c1[nH]c(=O)c(-c2cc(Cl)ccc2F)cc1Nc1ncnc2[nH]ccc12] | 1.86 |
| 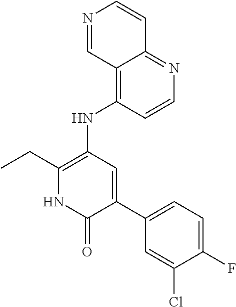 [SMILES: CCc1[nH]c(=O)c(-c2ccc(F)c(Cl)c2)cc1Nc1ccnc2ccncc12] | 1.87 |
| 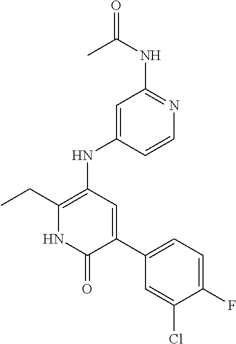 [SMILES: CCc1[nH]c(=O)c(-c2ccc(F)c(Cl)c2)cc1Nc1ccnc(NC(C)=O)c1] | 1.88 |
| 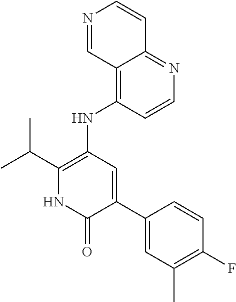 [SMILES: Cc1cc(-c2cc(Nc3ccnc4ccncc34)c(C(C)C)[nH]c2=O)ccc1F] | 1.89 |
| 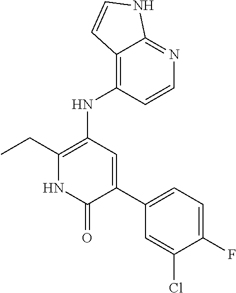 [SMILES: CCc1[nH]c(=O)c(-c2ccc(F)c(Cl)c2)cc1Nc1ccnc2[nH]ccc12] | 1.90 |

The compounds illustrated in table 2 can be prepared in a manner analogous to the techniques used in connection with the preparation of the table 1 compounds and in accordance, using appropriate, analogous starting materials and by utilizing the general synthetic schemes illustrated below.

TABLE 2
Compounds

| | |
|---|---|
| 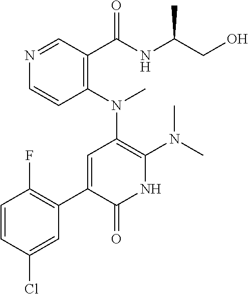 [SMILES: C[C@@H](CO)NC(=O)c1cnccc1N(C)c1cc(-c2cc(Cl)ccc2F)c(=O)[nH]c1N(C)C] | 2.1 |
| 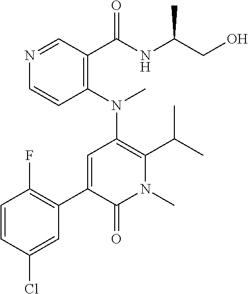 [SMILES: CC(C)c1c(N(C)c2ccncc2C(=O)N[C@@H](C)CO)cc(-c2cc(Cl)ccc2F)c(=O)n1C] | 2.2 |
| 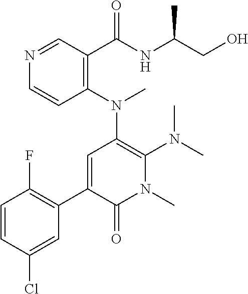 [SMILES: C[C@@H](CO)NC(=O)c1cnccc1N(C)c1cc(-c2cc(Cl)ccc2F)c(=O)n(C)c1N(C)C] | 2.3 |
| 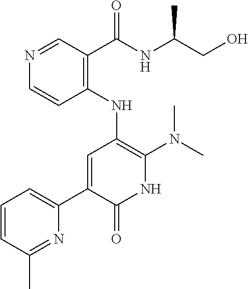 [SMILES: Cc1cccc(-c2cc(Nc3ccncc3C(=O)N[C@@H](C)CO)c(N(C)C)[nH]c2=O)n1] | 2.4 |
| 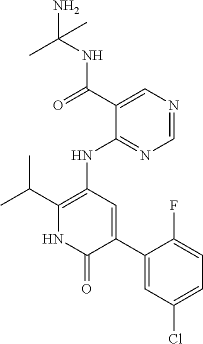 [SMILES: CC(C)c1[nH]c(=O)c(-c2cc(Cl)ccc2F)cc1Nc1ncncc1C(=O)NC(C)(C)N] | 2.5 |
| 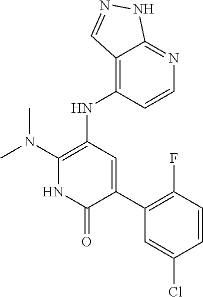 [SMILES: CN(C)c1[nH]c(=O)c(-c2cc(Cl)ccc2F)cc1Nc1ccnc2[nH]ncc12] | 2.6 |
| 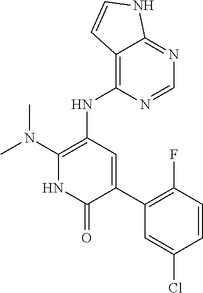 [SMILES: CN(C)c1[nH]c(=O)c(-c2cc(Cl)ccc2F)cc1Nc1ncnc2[nH]ccc12] | 2.7 |
| 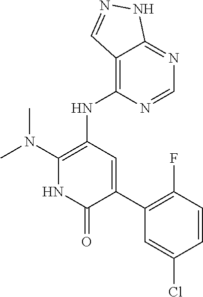 [SMILES: CN(C)c1[nH]c(=O)c(-c2cc(Cl)ccc2F)cc1Nc1ncnc2[nH]ncc12] | 2.8 |
| 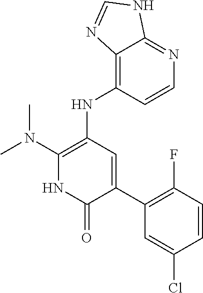 [SMILES: CN(C)c1[nH]c(=O)c(-c2cc(Cl)ccc2F)cc1Nc1ccnc2[nH]cnc12] | 2.9 |
| 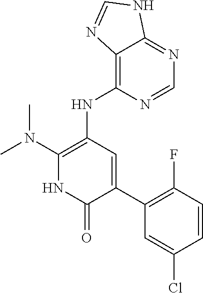 [SMILES: CN(C)c1[nH]c(=O)c(-c2cc(Cl)ccc2F)cc1Nc1ncnc2[nH]cnc12] | 2.10 |
| 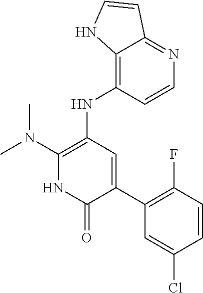 [SMILES: CN(C)c1[nH]c(=O)c(-c2cc(Cl)ccc2F)cc1Nc1ccnc2cc[nH]c12] | 2.11 |
| 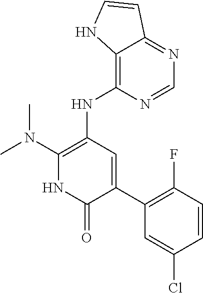 [SMILES: CN(C)c1[nH]c(=O)c(-c2cc(Cl)ccc2F)cc1Nc1ncnc2cc[nH]c12] | 2.12 |
| 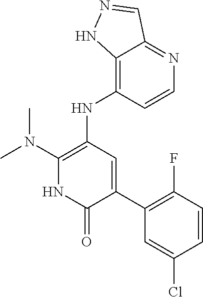 [SMILES: CN(C)c1[nH]c(=O)c(-c2cc(Cl)ccc2F)cc1Nc1ccnc2cn[nH]c12] | 2.13 |
| 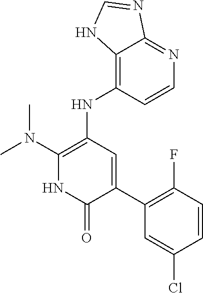 [SMILES: CN(C)c1[nH]c(=O)c(-c2cc(Cl)ccc2F)cc1Nc1ccnc2nc[nH]c12] | 2.14 |
| 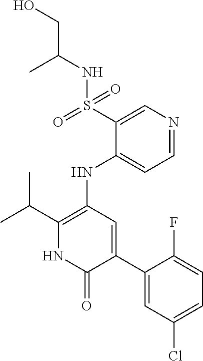 [SMILES: CC(CO)NS(=O)(=O)c1cnccc1Nc1cc(-c2cc(Cl)ccc2F)c(=O)[nH]c1C(C)C] | 2.15 |
| 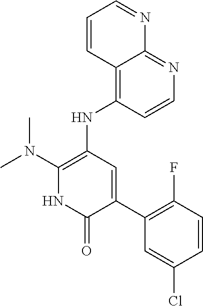 [SMILES: CN(C)c1[nH]c(=O)c(-c2cc(Cl)ccc2F)cc1Nc1ccnc2ncccc12] | 2.16 |
| 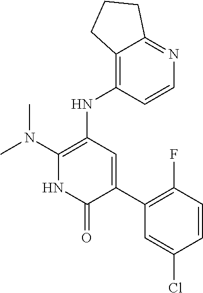 [SMILES: CN(C)c1[nH]c(=O)c(-c2cc(Cl)ccc2F)cc1Nc1ccnc2c1CCC2] | 2.17 |
| 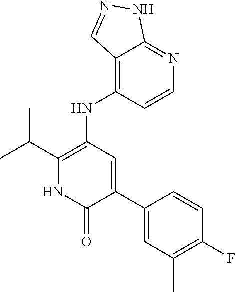 [SMILES: Cc1cc(-c2cc(Nc3ccnc4[nH]ncc34)c(C(C)C)[nH]c2=O)ccc1F] | 2.18 |
| 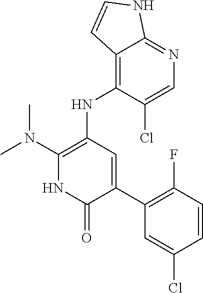 [SMILES: CN(C)c1[nH]c(=O)c(-c2cc(Cl)ccc2F)cc1Nc1c(Cl)cnc2[nH]ccc12] | 2.19 |
| 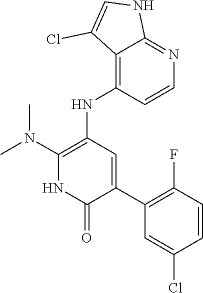 [SMILES: CN(C)c1[nH]c(=O)c(-c2cc(Cl)ccc2F)cc1Nc1ccnc2[nH]cc(Cl)c12] | 2.20 |
| 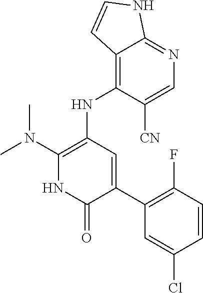 [SMILES: CN(C)c1[nH]c(=O)c(-c2cc(Cl)ccc2F)cc1Nc1c(C#N)cnc2[nH]ccc12] | 2.21 |
| 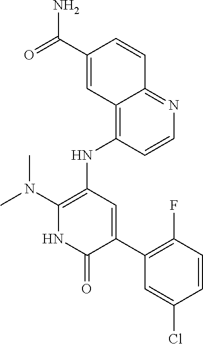 [SMILES: CN(C)c1[nH]c(=O)c(-c2cc(Cl)ccc2F)cc1Nc1ccnc2ccc(C(N)=O)cc12] | 2.22 |
| 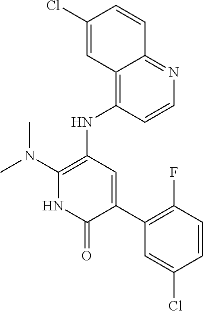 [SMILES: CN(C)c1[nH]c(=O)c(-c2cc(Cl)ccc2F)cc1Nc1ccnc2ccc(Cl)cc12] | 2.23 |
| 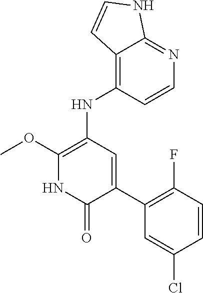 [SMILES: COc1[nH]c(=O)c(-c2cc(Cl)ccc2F)cc1Nc1ccnc2[nH]ccc12] | 2.24 |
| 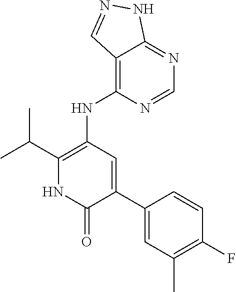 [SMILES: Cc1cc(-c2cc(Nc3ncnc4[nH]ncc34)c(C(C)C)[nH]c2=O)ccc1F] | 2.25 |
| 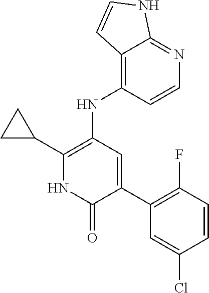 [SMILES: O=c1[nH]c(C2CC2)c(Nc2ccnc3[nH]ccc23)cc1-c1cc(Cl)ccc1F] | 2.26 |
| 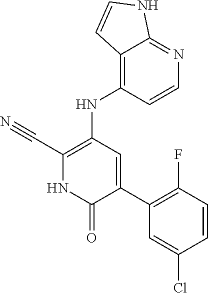 [SMILES: N#Cc1[nH]c(=O)c(-c2cc(Cl)ccc2F)cc1Nc1ccnc2[nH]ccc12] | 2.27 |
| 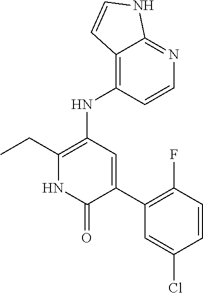 [SMILES: CCc1[nH]c(=O)c(-c2cc(Cl)ccc2F)cc1Nc1ccnc2[nH]ccc12] | 2.28 |
| 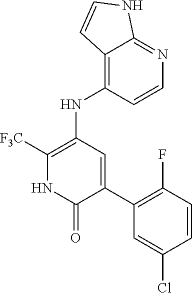 [SMILES: O=c1[nH]c(C(F)(F)F)c(Nc2ccnc3[nH]ccc23)cc1-c1cc(Cl)ccc1F] | 2.29 |
| 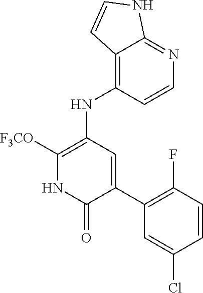 [SMILES: O=c1[nH]c(OC(F)(F)F)c(Nc2ccnc3[nH]ccc23)cc1-c1cc(Cl)ccc1F] | 2.30 |
| 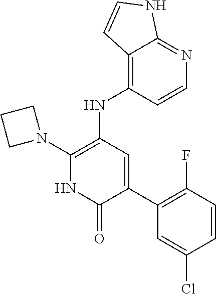 [SMILES: O=c1[nH]c(N2CCC2)c(Nc2ccnc3[nH]ccc23)cc1-c1cc(Cl)ccc1F] | 2.31 |
| 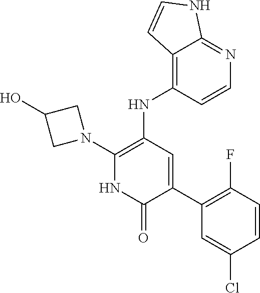 [SMILES: O=c1[nH]c(N2CC(O)C2)c(Nc2ccnc3[nH]ccc23)cc1-c1cc(Cl)ccc1F] | 2.32 |
| 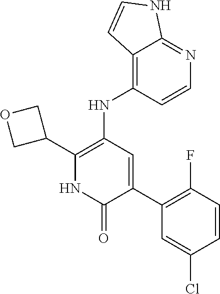 [SMILES: O=c1[nH]c(C2COC2)c(Nc2ccnc3[nH]ccc23)cc1-c1cc(Cl)ccc1F] | 2.33 |
| 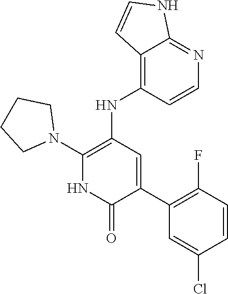 [SMILES: O=c1[nH]c(N2CCCC2)c(Nc2ccnc3[nH]ccc23)cc1-c1cc(Cl)ccc1F] | 2.34 |
| 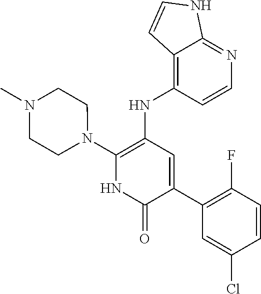 [SMILES: CN1CCN(c2[nH]c(=O)c(-c3cc(Cl)ccc3F)cc2Nc2ccnc3[nH]ccc23)CC1] | 2.35 |
| 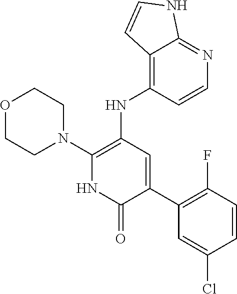 [SMILES: O=c1[nH]c(N2CCOCC2)c(Nc2ccnc3[nH]ccc23)cc1-c1cc(Cl)ccc1F] | 2.36 |
| 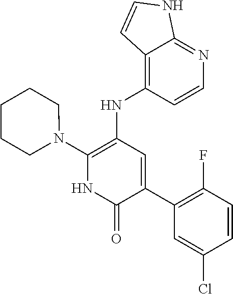 [SMILES: O=c1[nH]c(N2CCCCC2)c(Nc2ccnc3[nH]ccc23)cc1-c1cc(Cl)ccc1F] | 2.37 |
| 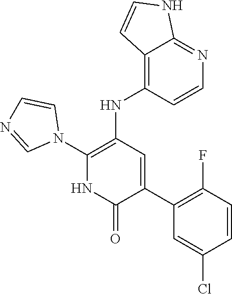 [SMILES: O=c1[nH]c(-n2ccnc2)c(Nc2ccnc3[nH]ccc23)cc1-c1cc(Cl)ccc1F] | 2.38 |
| 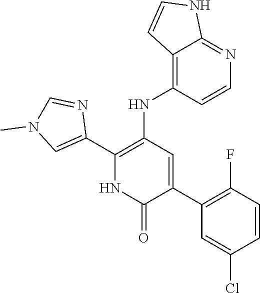 [SMILES: Cn1cnc(-c2[nH]c(=O)c(-c3cc(Cl)ccc3F)cc2Nc2ccnc3[nH]ccc23)c1] | 2.39 |
| 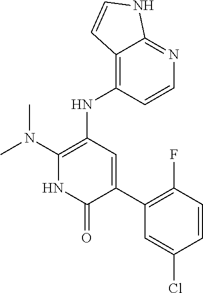 [SMILES: CN(C)c1[nH]c(=O)c(-c2cc(Cl)ccc2F)cc1Nc1ccnc2[nH]ccc12] | 2.40 |
| 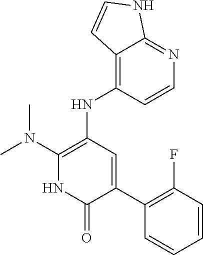 [SMILES: CN(C)c1[nH]c(=O)c(-c2ccccc2F)cc1Nc1ccnc2[nH]ccc12] | 2.41 |
| 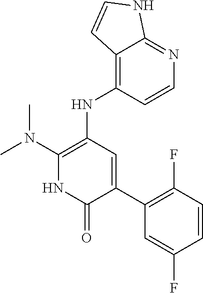 [SMILES: CN(C)c1[nH]c(=O)c(-c2cc(F)ccc2F)cc1Nc1ccnc2[nH]ccc12] | 2.42 |
| 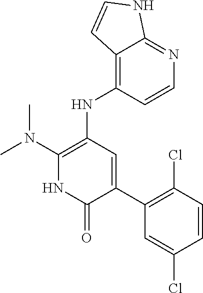 [SMILES: CN(C)c1[nH]c(=O)c(-c2cc(Cl)ccc2Cl)cc1Nc1ccnc2[nH]ccc12] | 2.43 |
| 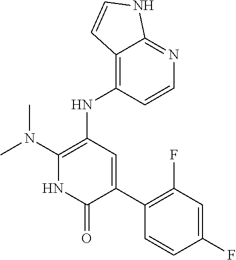 [SMILES: CN(C)c1[nH]c(=O)c(-c2ccc(F)cc2F)cc1Nc1ccnc2[nH]ccc12] | 2.44 |
| 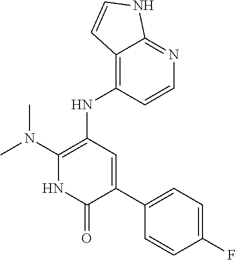 [SMILES: CN(C)c1[nH]c(=O)c(-c2ccc(F)cc2)cc1Nc1ccnc2[nH]ccc12] | 2.45 |
| 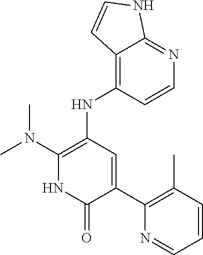 [SMILES: Cc1cccnc1-c1cc(Nc2ccnc3[nH]ccc23)c(N(C)C)[nH]c1=O] | 2.46 |
| 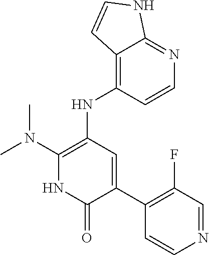 [SMILES: CN(C)c1[nH]c(=O)c(-c2ccncc2F)cc1Nc1ccnc2[nH]ccc12] | 2.47 |
| 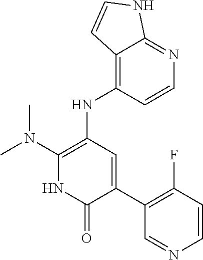 [SMILES: CN(C)c1[nH]c(=O)c(-c2cnccc2F)cc1Nc1ccnc2[nH]ccc12] | 2.48 |
| 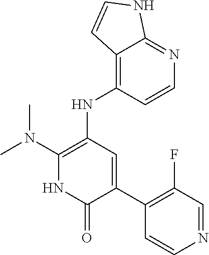 [SMILES: CN(C)c1[nH]c(=O)c(-c2ccncc2F)cc1Nc1ccnc2[nH]ccc12] | 2.49 |
| 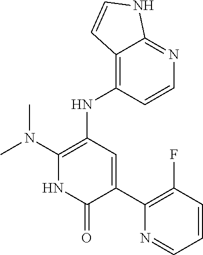 [SMILES: CN(C)c1[nH]c(=O)c(-c2ncccc2F)cc1Nc1ccnc2[nH]ccc12] | 2.50 |
| 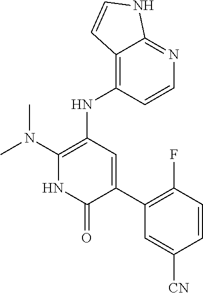 [SMILES: CN(C)c1[nH]c(=O)c(-c2cc(C#N)ccc2F)cc1Nc1ccnc2[nH]ccc12] | 2.51 |
| 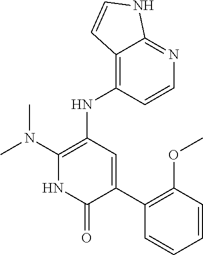 | 2.52 |
| 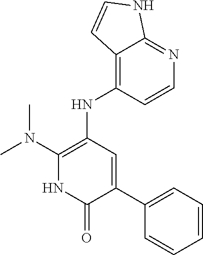 [SMILES: CN(C)c1[nH]c(=O)c(-c2ccccc2)cc1Nc1ccnc2[nH]ccc12] | 2.53 |
| 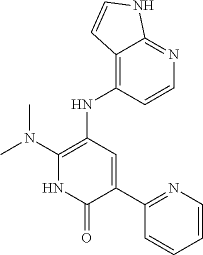 [SMILES: CN(C)c1[nH]c(=O)c(-c2ccccn2)cc1Nc1ccnc2[nH]ccc12] | 2.54 |
| 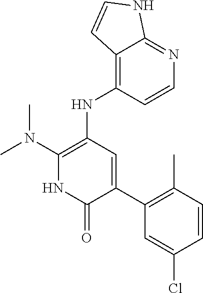 [SMILES: Cc1ccc(Cl)cc1-c1cc(Nc2ccnc3[nH]ccc23)c(N(C)C)[nH]c1=O] | 2.55 |
| 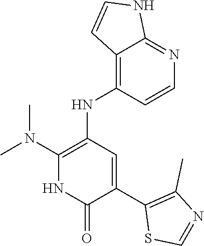 [SMILES: Cc1ncsc1-c1cc(Nc2ccnc3[nH]ccc23)c(N(C)C)[nH]c1=O] | 2.56 |
| 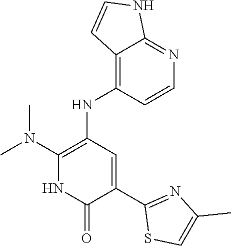 | 2.57 |
| 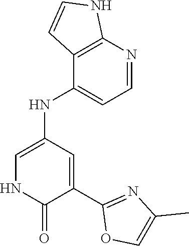 [SMILES: Cc1coc(-c2cc(Nc3ccnc4[nH]ccc34)c[nH]c2=O)n1] | 2.58 |
| 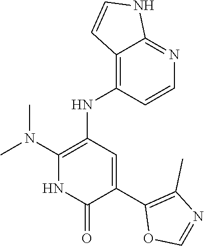 [SMILES: Cc1ncoc1-c1cc(Nc2ccnc3[nH]ccc23)c(N(C)C)[nH]c1=O] | 2.59 |
| 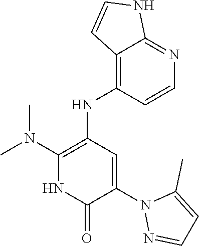 [SMILES: Cc1ccnn1-c1cc(Nc2ccnc3[nH]ccc23)c(N(C)C)[nH]c1=O] | 2.60 |
| 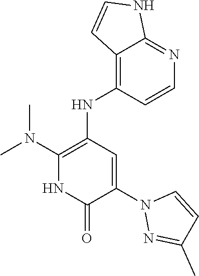 [SMILES: Cc1ccn(-c2cc(Nc3ccnc4[nH]ccc34)c(N(C)C)[nH]c2=O)n1] | 2.61 |
| 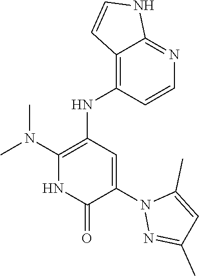 [SMILES: Cc1cc(C)n(-c2cc(Nc3ccnc4[nH]ccc34)c(N(C)C)[nH]c2=O)n1] | 2.62 |
| 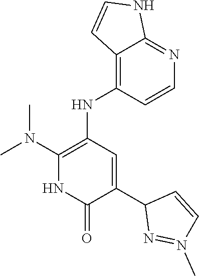 | 2.63 |
| 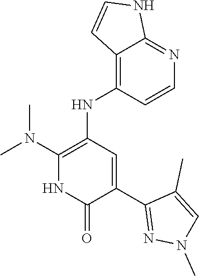 [SMILES: Cc1cn(C)nc1-c1cc(Nc2ccnc3[nH]ccc23)c(N(C)C)[nH]c1=O] | 2.64 |
| 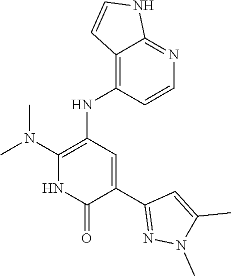 | 2.65 |
| 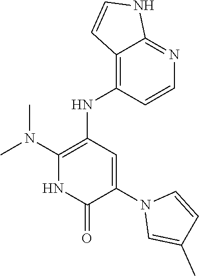 [SMILES: Cc1ccn(-c2cc(Nc3ccnc4[nH]ccc34)c(N(C)C)[nH]c2=O)c1] | 2.66 |
| 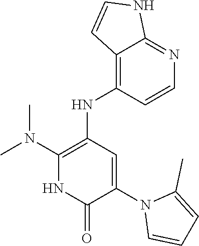 [SMILES: Cc1cccn1-c1cc(Nc2ccnc3[nH]ccc23)c(N(C)C)[nH]c1=O] | 2.67 |
| 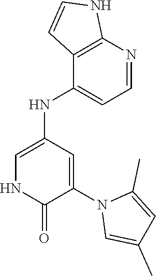 [SMILES: Cc1cc(C)n(-c2cc(Nc3ccnc4[nH]ccc34)c[nH]c2=O)c1] | 2.68 |
| 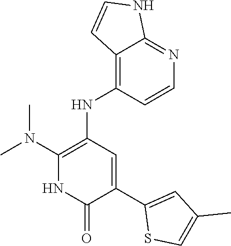 | 2.69 |
| 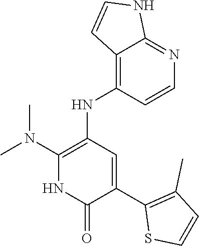 [SMILES: Cc1ccsc1-c1cc(Nc2ccnc3[nH]ccc23)c(N(C)C)[nH]c1=O] | 2.70 |
| 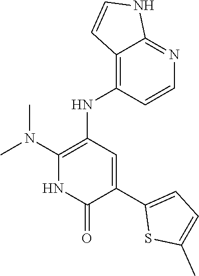 [SMILES: Cc1ccc(-c2cc(Nc3ccnc4[nH]ccc34)c(N(C)C)[nH]c2=O)s1] | 2.71 |
| 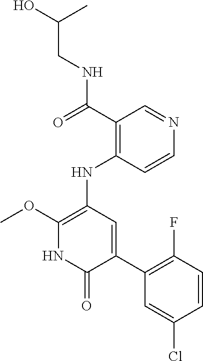 [SMILES: COc1[nH]c(=O)c(-c2cc(Cl)ccc2F)cc1Nc1ccncc1C(=O)NCC(C)O] | 2.72 |
| 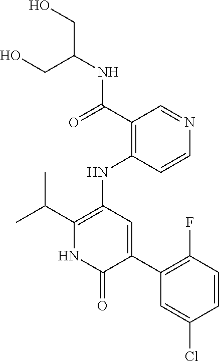 [SMILES: CC(C)c1[nH]c(=O)c(-c2cc(Cl)ccc2F)cc1Nc1ccncc1C(=O)NC(CO)CO] | 2.73 |
| 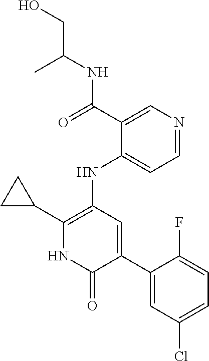 [SMILES: CC(CO)NC(=O)c1cnccc1Nc1cc(-c2cc(Cl)ccc2F)c(=O)[nH]c1C1CC1] | 2.74 |
| 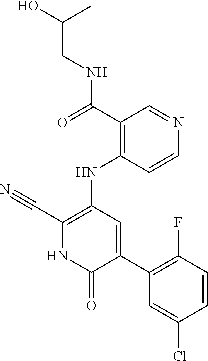 [SMILES: CC(O)CNC(=O)c1cnccc1Nc1cc(-c2cc(Cl)ccc2F)c(=O)[nH]c1C#N] | 2.75 |
| 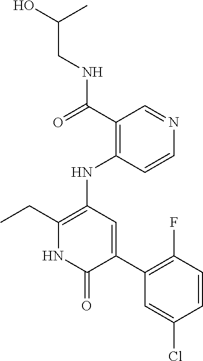 [SMILES: CCc1[nH]c(=O)c(-c2cc(Cl)ccc2F)cc1Nc1ccncc1C(=O)NCC(C)O] | 2.76 |
| 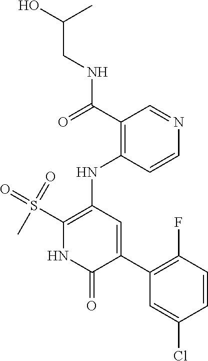 [SMILES: CC(O)CNC(=O)c1cnccc1Nc1cc(-c2cc(Cl)ccc2F)c(=O)[nH]c1S(C)(=O)=O] | 2.77 |
| 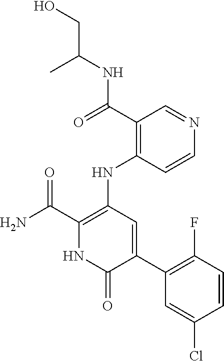 [SMILES: CC(CO)NC(=O)c1cnccc1Nc1cc(-c2cc(Cl)ccc2F)c(=O)[nH]c1C(N)=O] | 2.78 |
| 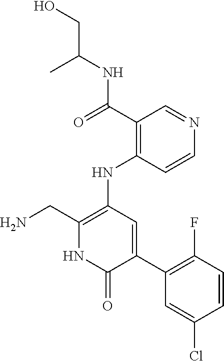 [SMILES: CC(CO)NC(=O)c1cnccc1Nc1cc(-c2cc(Cl)ccc2F)c(=O)[nH]c1CN] | 2.79 |
| 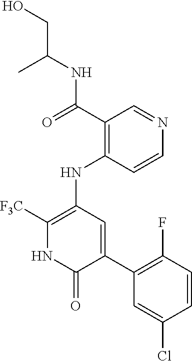 [SMILES: CC(CO)NC(=O)c1cnccc1Nc1cc(-c2cc(Cl)ccc2F)c(=O)[nH]c1C(F)(F)F] | 2.80 |
| 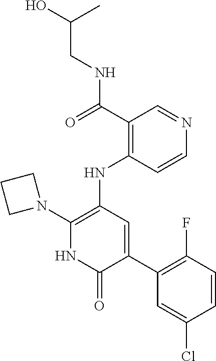 [SMILES: CC(O)CNC(=O)c1cnccc1Nc1cc(-c2cc(Cl)ccc2F)c(=O)[nH]c1N1CCC1] | 2.81 |
| 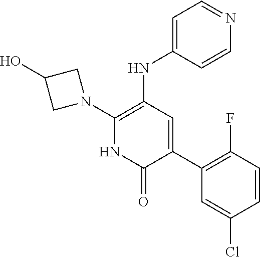 [SMILES: O=c1[nH]c(N2CC(O)C2)c(Nc2ccncc2)cc1-c1cc(Cl)ccc1F] | 2.82 |
| 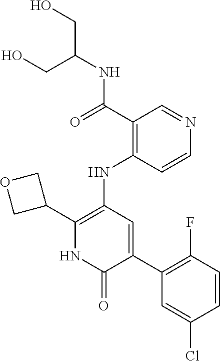 [SMILES: O=C(NC(CO)CO)c1cnccc1Nc1cc(-c2cc(Cl)ccc2F)c(=O)[nH]c1C1COC1] | 2.83 |
| 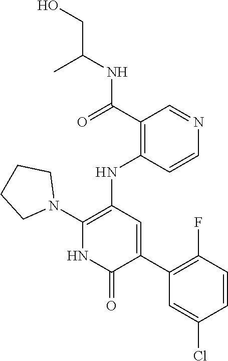 [SMILES: CC(CO)NC(=O)c1cnccc1Nc1cc(-c2cc(Cl)ccc2F)c(=O)[nH]c1N1CCCC1] | 2.84 |
| 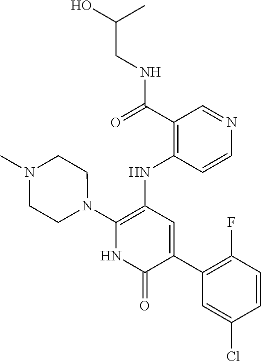 [SMILES: CC(O)CNC(=O)c1cnccc1Nc1cc(-c2cc(Cl)ccc2F)c(=O)[nH]c1N1CCN(C)CC1] | 2.85 |
| 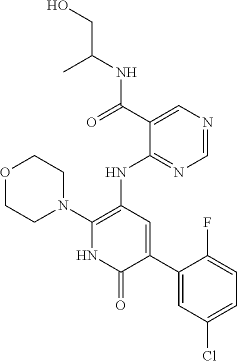 [SMILES: CC(CO)NC(=O)c1cncnc1Nc1cc(-c2cc(Cl)ccc2F)c(=O)[nH]c1N1CCOCC1] | 2.86 |
| 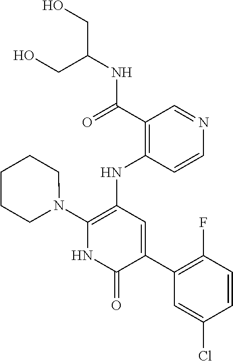 [SMILES: O=C(NC(CO)CO)c1cnccc1Nc1cc(-c2cc(Cl)ccc2F)c(=O)[nH]c1N1CCCCC1] | 2.87 |
| 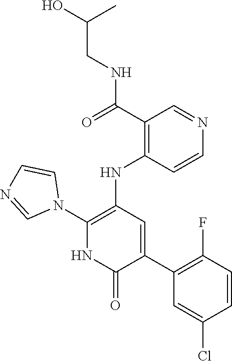 [SMILES: CC(O)CNC(=O)c1cnccc1Nc1cc(-c2cc(Cl)ccc2F)c(=O)[nH]c1-n1ccnc1] | 2.88 |
| 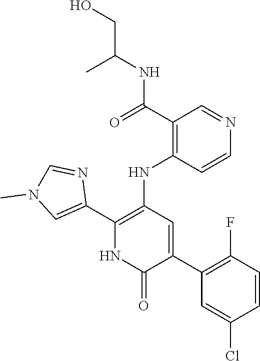 [SMILES: CC(CO)NC(=O)c1cnccc1Nc1cc(-c2cc(Cl)ccc2F)c(=O)[nH]c1-c1cn(C)cn1] | 2.89 |
| 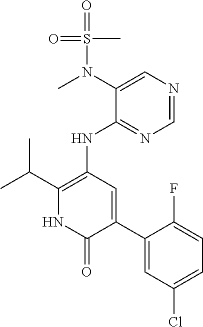 [SMILES: CC(C)c1[nH]c(=O)c(-c2cc(Cl)ccc2F)cc1Nc1ncncc1N(C)S(C)(=O)=O] | 2.90 |
| 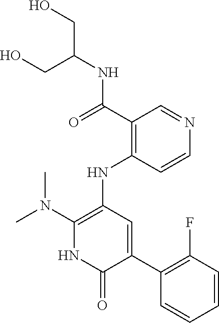 [SMILES: CN(C)c1[nH]c(=O)c(-c2ccccc2F)cc1Nc1ccncc1C(=O)NC(CO)CO] | 2.91 |
| 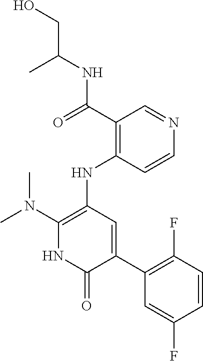 [SMILES: CC(CO)NC(=O)c1cnccc1Nc1cc(-c2cc(F)ccc2F)c(=O)[nH]c1N(C)C] | 2.92 |
| 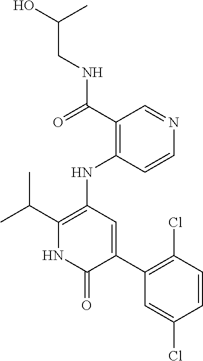 [SMILES: CC(O)CNC(=O)c1cnccc1Nc1cc(-c2cc(Cl)ccc2Cl)c(=O)[nH]c1C(C)C] | 2.93 |
| 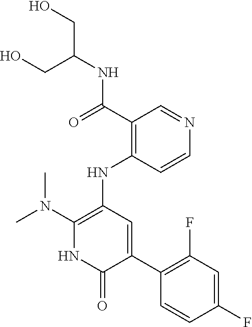 [SMILES: CN(C)c1[nH]c(=O)c(-c2ccc(F)cc2F)cc1Nc1ccncc1C(=O)NC(CO)CO] | 2.94 |
| 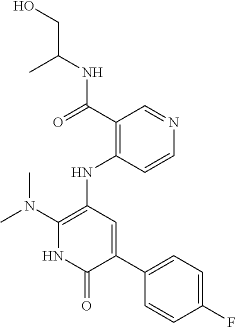 [SMILES: CC(CO)NC(=O)c1cnccc1Nc1cc(-c2ccc(F)cc2)c(=O)[nH]c1N(C)C] | 2.95 |
| 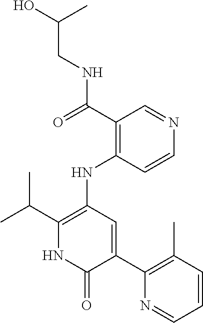 [SMILES: Cc1cccnc1-c1cc(Nc2ccncc2C(=O)NCC(C)O)c(C(C)C)[nH]c1=O] | 2.96 |
| 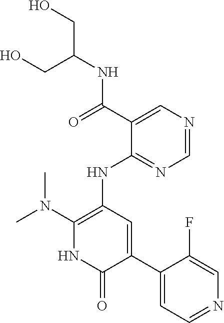 [SMILES: CN(C)c1[nH]c(=O)c(-c2ccncc2F)cc1Nc1ncncc1C(=O)NC(CO)CO] | 2.97 |
| 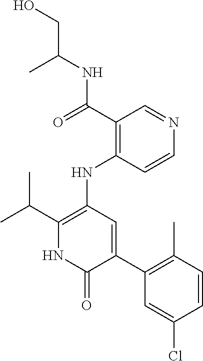 [SMILES: Cc1ccc(Cl)cc1-c1cc(Nc2ccncc2C(=O)NC(C)CO)c(C(C)C)[nH]c1=O] | 2.98 |
| 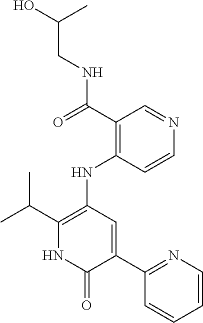 [SMILES: CC(O)CNC(=O)c1cnccc1Nc1cc(-c2ccccn2)c(=O)[nH]c1C(C)C] | 2.99 |
| 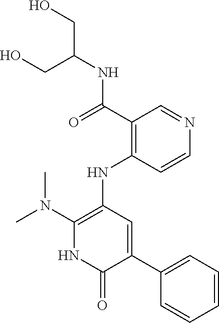 [SMILES: CN(C)c1[nH]c(=O)c(-c2ccccc2)cc1Nc1ccncc1C(=O)NC(CO)CO] | 2.100 |
| 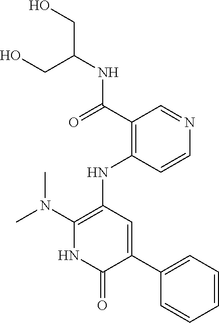 [SMILES: CN(C)c1[nH]c(=O)c(-c2ccccc2)cc1Nc1ccncc1C(=O)NC(CO)CO] | 2.101 |
| 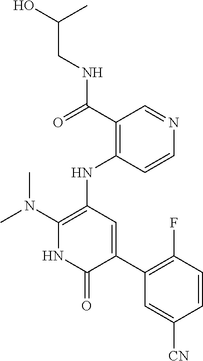 [SMILES: CC(O)CNC(=O)c1cnccc1Nc1cc(-c2cc(C#N)ccc2F)c(=O)[nH]c1N(C)C] | 2.102 |
| 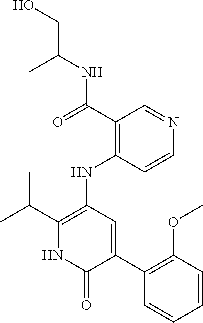 [SMILES: COc1ccccc1-c1cc(Nc2ccncc2C(=O)NC(C)CO)c(C(C)C)[nH]c1=O] | 2.103 |
| 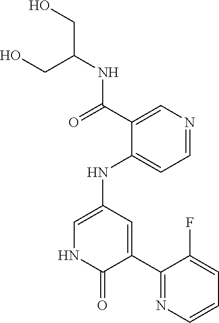 [SMILES: O=C(NC(CO)CO)c1cnccc1Nc1c[nH]c(=O)c(-c2ncccc2F)c1] | 2.104 |
| 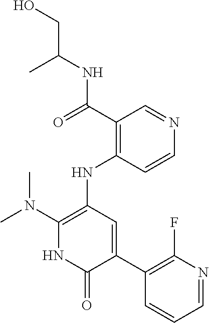 [SMILES: CC(CO)NC(=O)c1cnccc1Nc1cc(-c2cccnc2F)c(=O)[nH]c1N(C)C] | 2.105 |
| 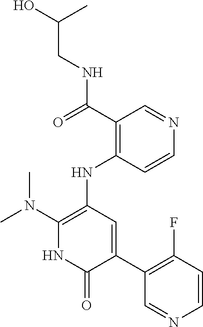 [SMILES: CC(O)CNC(=O)c1cnccc1Nc1cc(-c2cnccc2F)c(=O)[nH]c1N(C)C] | 2.106 |
| 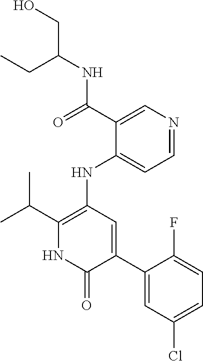 [SMILES: CCC(CO)NC(=O)c1cnccc1Nc1cc(-c2cc(Cl)ccc2F)c(=O)[nH]c1C(C)C] | 2.107 |
| 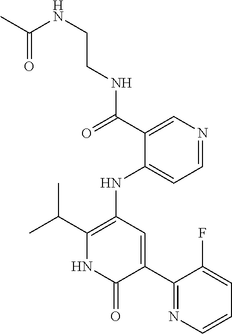 [SMILES: CC(=O)NCCNC(=O)c1cnccc1Nc1cc(-c2ncccc2F)c(=O)[nH]c1C(C)C] | 2.108 |
| 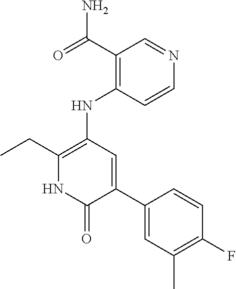 [SMILES: CCc1[nH]c(=O)c(-c2ccc(F)c(C)c2)cc1Nc1ccncc1C(N)=O] | 2.109 |
| 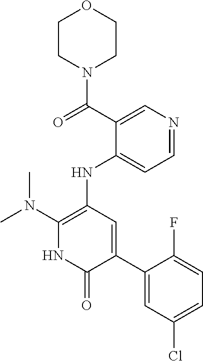 [SMILES: CN(C)c1[nH]c(=O)c(-c2cc(Cl)ccc2F)cc1Nc1ccncc1C(=O)N1CCOCC1] | 2.110 |
| 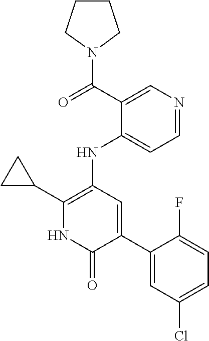 [SMILES: O=C(c1cnccc1Nc1cc(-c2cc(Cl)ccc2F)c(=O)[nH]c1C1CC1)N1CCCC1] | 2.111 |
| 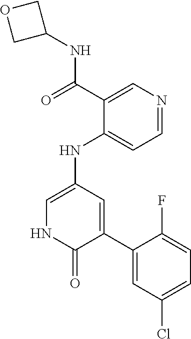 [SMILES: O=C(NC1COC1)c1cnccc1Nc1c[nH]c(=O)c(-c2cc(Cl)ccc2F)c1] | 2.112 |
| 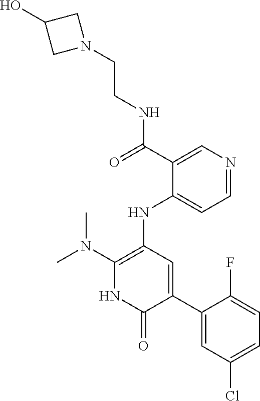 [SMILES: CN(C)c1[nH]c(=O)c(-c2cc(Cl)ccc2F)cc1Nc1ccncc1C(=O)NCCN1CC(O)C1] | 2.113 |
| 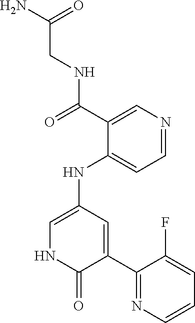 [SMILES: NC(=O)CNC(=O)c1cnccc1Nc1c[nH]c(=O)c(-c2ncccc2F)c1] | 2.114 |
| 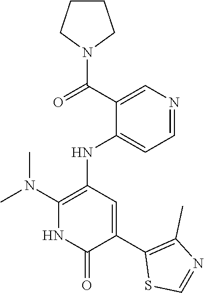 [SMILES: Cc1ncsc1-c1cc(Nc2ccncc2C(=O)N2CCCC2)c(N(C)C)[nH]c1=O] | 2.115 |
| 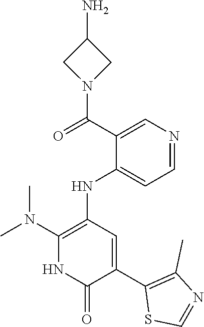 [SMILES: Cc1ncsc1-c1cc(Nc2ccncc2C(=O)N2CC(N)C2)c(N(C)C)[nH]c1=O] | 2.116 |
| 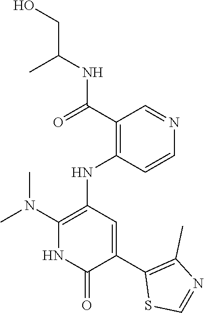 [SMILES: Cc1ncsc1-c1cc(Nc2ccncc2C(=O)NC(C)CO)c(N(C)C)[nH]c1=O] | 2.117 |
| 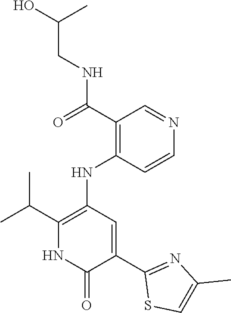 [SMILES: Cc1csc(-c2cc(Nc3ccncc3C(=O)NCC(C)O)c(C(C)C)[nH]c2=O)n1] | 2.118 |
| 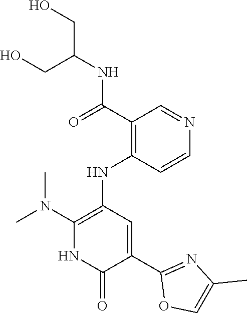 | 2.119 |
| 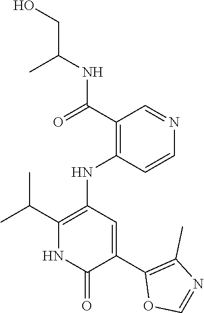 [SMILES: Cc1ncoc1-c1cc(Nc2ccncc2C(=O)NC(C)CO)c(C(C)C)[nH]c1=O] | 2.120 |
| 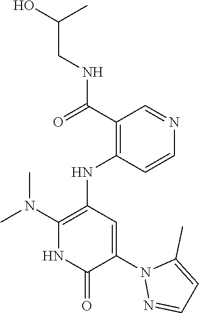 [SMILES: Cc1ccnn1-c1cc(Nc2ccncc2C(=O)NCC(C)O)c(N(C)C)[nH]c1=O] | 2.121 |
| 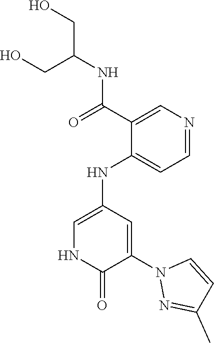 [SMILES: Cc1ccn(-c2cc(Nc3ccncc3C(=O)NC(CO)CO)c[nH]c2=O)n1] | 2.122 |
| 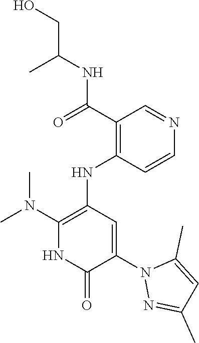 [SMILES: Cc1cc(C)n(-c2cc(Nc3ccncc3C(=O)NC(C)CO)c(N(C)C)[nH]c2=O)n1] | 2.123 |
| 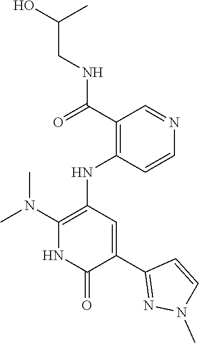 [SMILES: CC(O)CNC(=O)c1cnccc1Nc1cc(-c2ccn(C)n2)c(=O)[nH]c1N(C)C] | 2.124 |
| 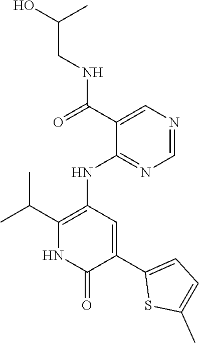 [SMILES: Cc1ccc(-c2cc(Nc3ncncc3C(=O)NCC(C)O)c(C(C)C)[nH]c2=O)s1] | 2.125 |
| 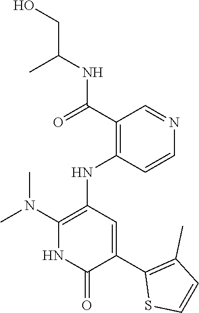 [SMILES: Cc1ccsc1-c1cc(Nc2ccncc2C(=O)NC(C)CO)c(N(C)C)[nH]c1=O] | 2.126 |
| 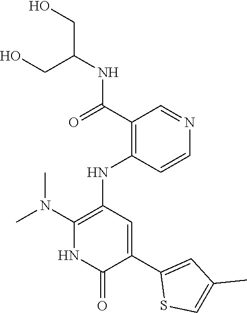 | 2.127 |
| 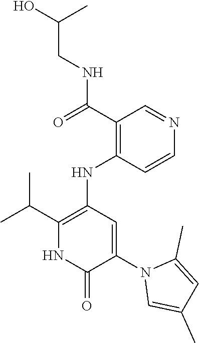 [SMILES: Cc1cc(C)n(-c2cc(Nc3ccncc3C(=O)NCC(C)O)c(C(C)C)[nH]c2=O)c1] | 2.128 |
| 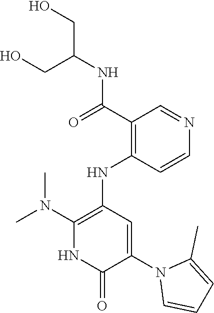 [SMILES: Cc1cccn1-c1cc(Nc2ccncc2C(=O)NC(CO)CO)c(N(C)C)[nH]c1=O] | 2.129 |
| 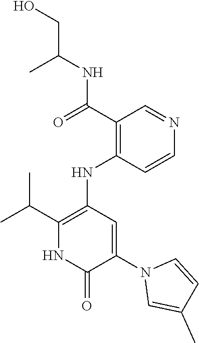 [SMILES: Cc1ccn(-c2cc(Nc3ccncc3C(=O)NC(C)CO)c(C(C)C)[nH]c2=O)c1] | 2.130 |
| 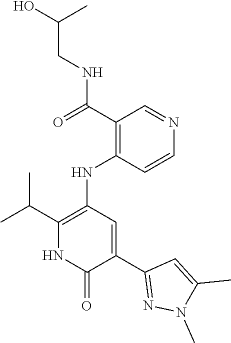 [SMILES: Cc1cc(-c2cc(Nc3ccncc3C(=O)NCC(C)O)c(C(C)C)[nH]c2=O)nn1C] | 2.131 |
| 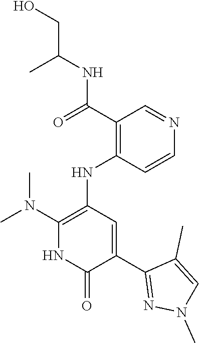 [SMILES: Cc1cn(C)nc1-c1cc(Nc2ccncc2C(=O)NC(C)CO)c(N(C)C)[nH]c1=O] | 2.132 |
| 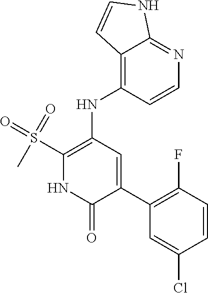 [SMILES: CS(=O)(=O)c1[nH]c(=O)c(-c2cc(Cl)ccc2F)cc1Nc1ccnc2[nH]ccc12] | 2.133 |
| 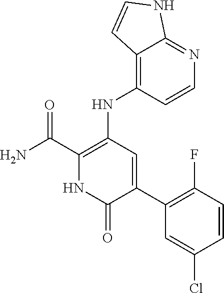 [SMILES: NC(=O)c1[nH]c(=O)c(-c2cc(Cl)ccc2F)cc1Nc1ccnc2[nH]ccc12] | 2.134 |
| 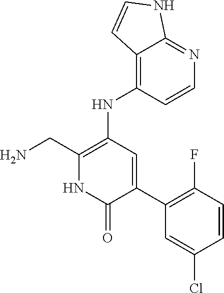 [SMILES: NCc1[nH]c(=O)c(-c2cc(Cl)ccc2F)cc1Nc1ccnc2[nH]ccc12] | 2.135 |
| 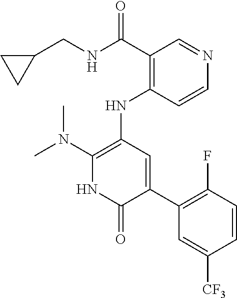 [SMILES: CN(C)c1[nH]c(=O)c(-c2cc(C(F)(F)F)ccc2F)cc1Nc1ccncc1C(=O)NCC1CC1] | 2.136 |
| 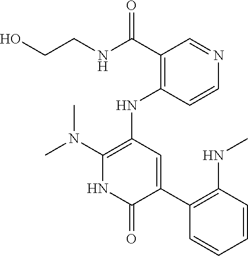 [SMILES: CNc1ccccc1-c1cc(Nc2ccncc2C(=O)NCCO)c(N(C)C)[nH]c1=O] | 2.137 |
| 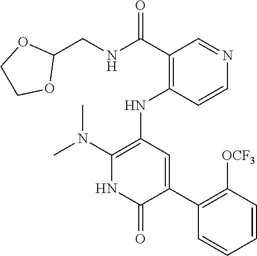 [SMILES: CN(C)c1[nH]c(=O)c(-c2ccccc2OC(F)(F)F)cc1Nc1ccncc1C(=O)NCC1OCCO1] | 2.138 |
| 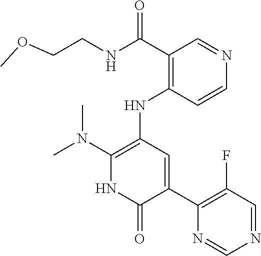 [SMILES: COCCNC(=O)c1cnccc1Nc1cc(-c2ncncc2F)c(=O)[nH]c1N(C)C] | 2.139 |
| 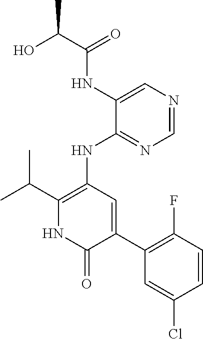 [SMILES: CC(C)c1[nH]c(=O)c(-c2cc(Cl)ccc2F)cc1Nc1ncncc1NC(=O)[C@H](C)O] | 2.140 |
| 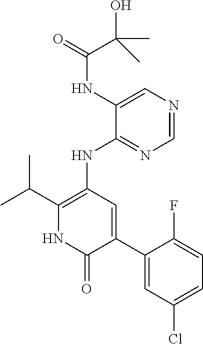 [SMILES: CC(C)c1[nH]c(=O)c(-c2cc(Cl)ccc2F)cc1Nc1ncncc1NC(=O)C(C)(C)O] | 2.141 |
| 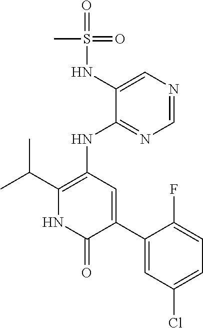 [SMILES: CC(C)c1[nH]c(=O)c(-c2cc(Cl)ccc2F)cc1Nc1ncncc1NS(C)(=O)=O] | 2.142 |
| 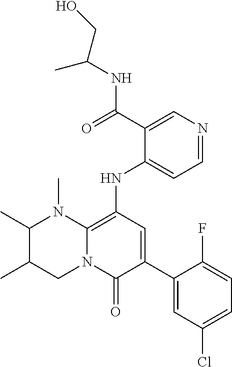 [SMILES: CC(CO)NC(=O)c1cnccc1Nc1cc(-c2cc(Cl)ccc2F)c(=O)n2c1N(C)C(C)C(C)C2] | 2.143 |
| 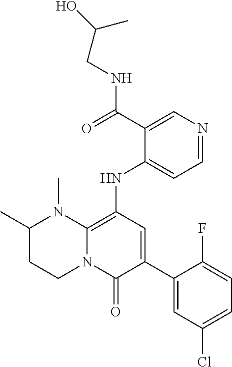 [SMILES: CC(O)CNC(=O)c1cnccc1Nc1cc(-c2cc(Cl)ccc2F)c(=O)n2c1N(C)C(C)CC2] | 2.144 |
| 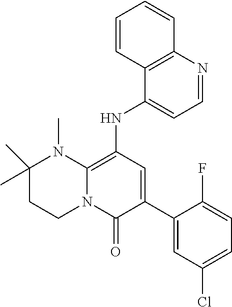 [SMILES: CN1c2c(Nc3ccnc4ccccc34)cc(-c3cc(Cl)ccc3F)c(=O)n2CCC1(C)C] | 2.145 |
| 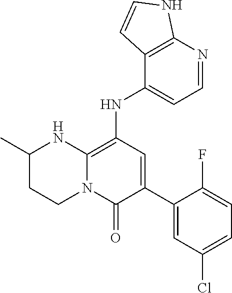 [SMILES: CC1CCn2c(c(Nc3ccnc4[nH]ccc34)cc(-c3cc(Cl)ccc3F)c2=O)N1] | 2.146 |
| 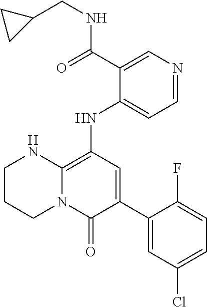 [SMILES: O=C(NCC1CC1)c1cnccc1Nc1cc(-c2cc(Cl)ccc2F)c(=O)n2c1NCCC2] | 2.147 |
| 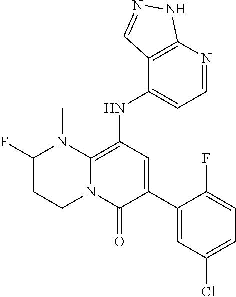 [SMILES: CN1c2c(Nc3ccnc4[nH]ncc34)cc(-c3cc(Cl)ccc3F)c(=O)n2CCC1F] | 2.148 |
| 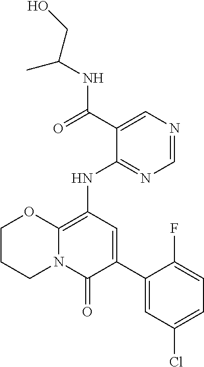 [SMILES: CC(CO)NC(=O)c1cncnc1Nc1cc(-c2cc(Cl)ccc2F)c(=O)n2c1OCCC2] | 2.149 |
| 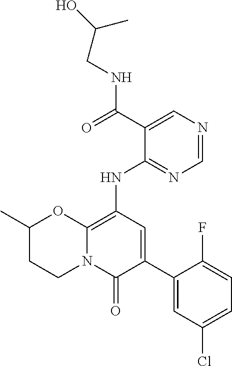 [SMILES: CC(O)CNC(=O)c1cncnc1Nc1cc(-c2cc(Cl)ccc2F)c(=O)n2c1OC(C)CC2] | 2.150 |
| 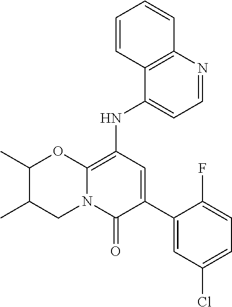 [SMILES: CC1Cn2c(c(Nc3ccnc4ccccc34)cc(-c3cc(Cl)ccc3F)c2=O)OC1C] | 2.151 |
| 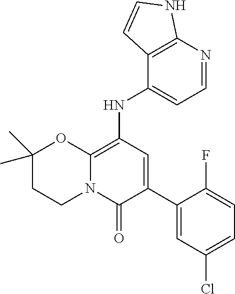 [SMILES: CC1(C)CCn2c(c(Nc3ccnc4[nH]ccc34)cc(-c3cc(Cl)ccc3F)c2=O)O1] | 2.152 |
| 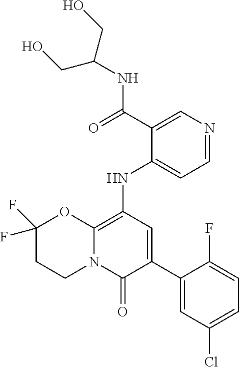 [SMILES: O=C(NC(CO)CO)c1cnccc1Nc1cc(-c2cc(Cl)ccc2F)c(=O)n2c1OC(F)(F)CC2] | 2.153 |
| 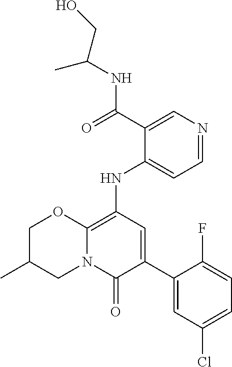 [SMILES: CC1COc2c(Nc3ccncc3C(=O)NC(C)CO)cc(-c3cc(Cl)ccc3F)c(=O)n2C1] | 2.154 |
| 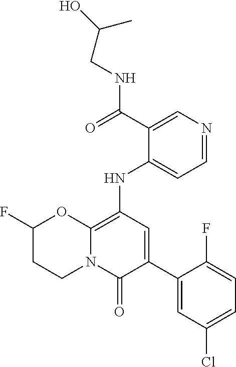 [SMILES: CC(O)CNC(=O)c1cnccc1Nc1cc(-c2cc(Cl)ccc2F)c(=O)n2c1OC(F)CC2] | 2.155 |
| 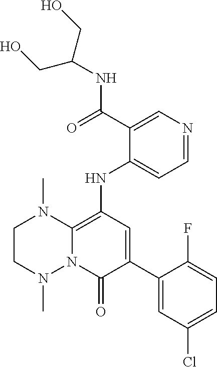 [SMILES: CN1CCN(C)n2c1c(Nc1ccncc1C(=O)NC(CO)CO)cc(-c1cc(Cl)ccc1F)c2=O] | 2.156 |
| 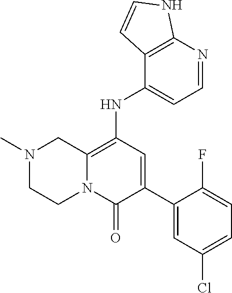 [SMILES: CN1CCn2c(c(Nc3ccnc4[nH]ccc34)cc(-c3cc(Cl)ccc3F)c2=O)C1] | 2.157 |
| 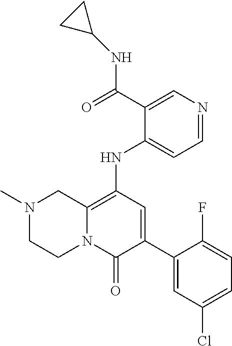 [SMILES: CN1CCn2c(c(Nc3ccncc3C(=O)NC3CC3)cc(-c3cc(Cl)ccc3F)c2=O)C1] | 2.158 |
| 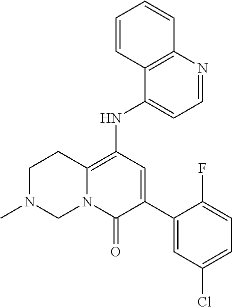 [SMILES: CN1CCc2c(Nc3ccnc4ccccc34)cc(-c3cc(Cl)ccc3F)c(=O)n2C1] | 2.159 |
| 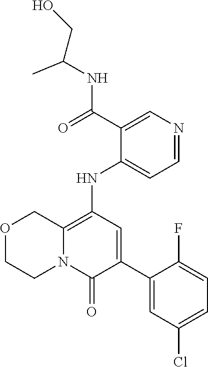 [SMILES: CC(CO)NC(=O)c1cnccc1Nc1cc(-c2cc(Cl)ccc2F)c(=O)n2c1COCC2] | 2.160 |
| 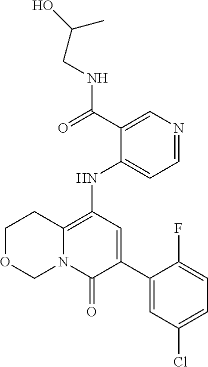 [SMILES: CC(O)CNC(=O)c1cnccc1Nc1cc(-c2cc(Cl)ccc2F)c(=O)n2c1CCOC2] | 2.161 |
| 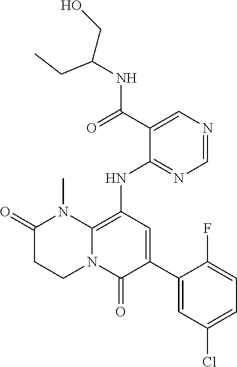 [SMILES: CCC(CO)NC(=O)c1cncnc1Nc1cc(-c2cc(Cl)ccc2F)c(=O)n2c1N(C)C(=O)CC2] | 2.162 |
| 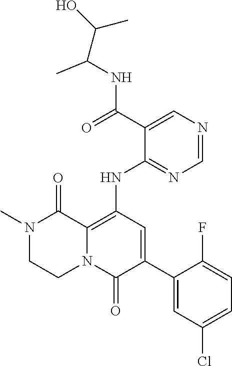 [SMILES: CC(O)C(C)NC(=O)c1cncnc1Nc1cc(-c2cc(Cl)ccc2F)c(=O)n2c1C(=O)N(C)CC2] | 2.163 |
| 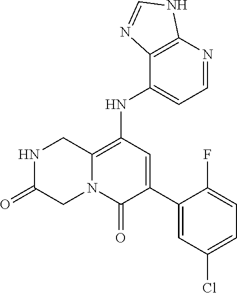 [SMILES: O=C1Cn2c(c(Nc3ccnc4[nH]cnc34)cc(-c3cc(Cl)ccc3F)c2=O)CN1] | 2.164 |
| 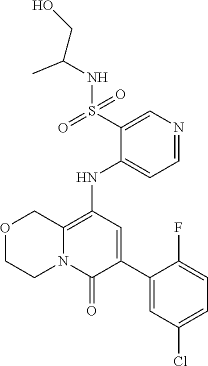 [SMILES: CC(CO)NS(=O)(=O)c1cnccc1Nc1cc(-c2cc(Cl)ccc2F)c(=O)n2c1COCC2] | 2.165 |
| 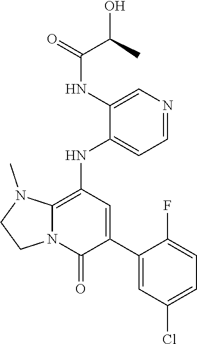 [SMILES: C[C@H](O)C(=O)Nc1cnccc1Nc1cc(-c2cc(Cl)ccc2F)c(=O)n2c1N(C)CC2] | 2.166 |
| 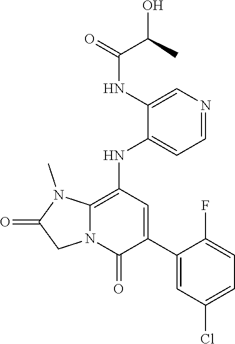 [SMILES: C[C@H](O)C(=O)Nc1cnccc1Nc1cc(-c2cc(Cl)ccc2F)c(=O)n2c1N(C)C(=O)C2] | 2.167 |
| 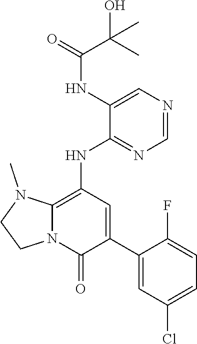 [SMILES: CN1CCn2c1c(Nc1ncncc1NC(=O)C(C)(C)O)cc(-c1cc(Cl)ccc1F)c2=O] | 2.168 |
| 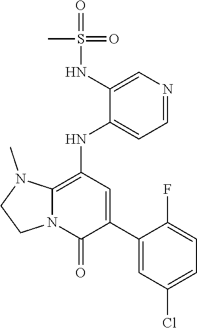 [SMILES: CN1CCn2c1c(Nc1ccncc1NS(C)(=O)=O)cc(-c1cc(Cl)ccc1F)c2=O] | 2.169 |
| 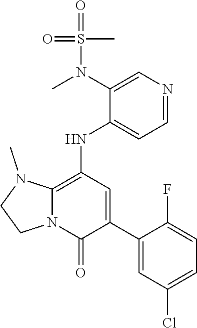 [SMILES: CN1CCn2c1c(Nc1ccncc1N(C)S(C)(=O)=O)cc(-c1cc(Cl)ccc1F)c2=O] | 2.170 |
| 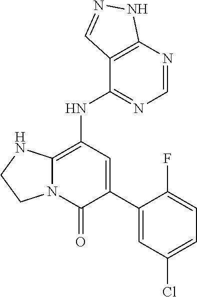 [SMILES: O=c1c(-c2cc(Cl)ccc2F)cc(Nc2ncnc3[nH]ncc23)c2n1CCN2] | 2.171 |
| 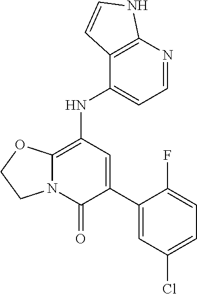 [SMILES: O=c1c(-c2cc(Cl)ccc2F)cc(Nc2ccnc3[nH]ccc23)c2n1CCO2] | 2.172 |
| 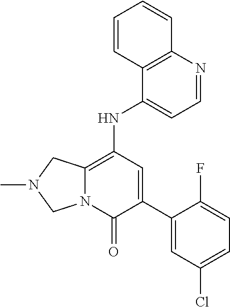 [SMILES: CN1Cc2c(Nc3ccnc4ccccc34)cc(-c3cc(Cl)ccc3F)c(=O)n2C1] | 2.173 |
| 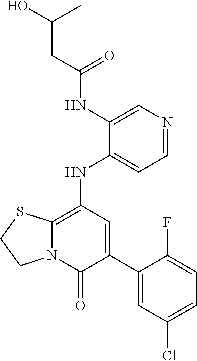 [SMILES: CC(O)CC(=O)Nc1cnccc1Nc1cc(-c2cc(Cl)ccc2F)c(=O)n2c1SCC2] | 2.174 |
| 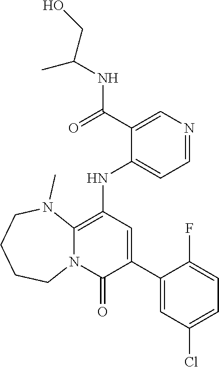 [SMILES: CC(CO)NC(=O)c1cnccc1Nc1cc(-c2cc(Cl)ccc2F)c(=O)n2c1N(C)CCCC2] | 2.175 |
| 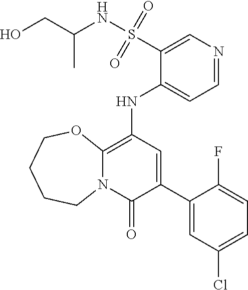 [SMILES: CC(CO)NS(=O)(=O)c1cnccc1Nc1cc(-c2cc(Cl)ccc2F)c(=O)n2c1OCCCC2] | 2.176 |
| 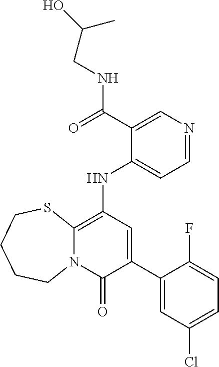 [SMILES: CC(O)CNC(=O)c1cnccc1Nc1cc(-c2cc(Cl)ccc2F)c(=O)n2c1SCCCC2] | 2.177 |
| 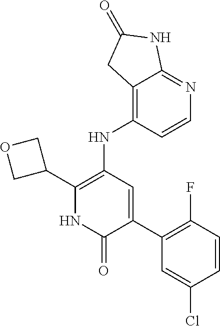 [SMILES: O=C1Cc2c(Nc3cc(-c4cc(Cl)ccc4F)c(=O)[nH]c3C3COC3)ccnc2N1] | 2.178 |
| 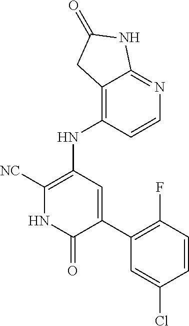 [SMILES: N#Cc1[nH]c(=O)c(-c2cc(Cl)ccc2F)cc1Nc1ccnc2c1CC(=O)N2] | 2.179 |
| 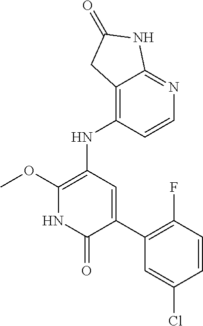 [SMILES: COc1[nH]c(=O)c(-c2cc(Cl)ccc2F)cc1Nc1ccnc2c1CC(=O)N2] | 2.180 |
| 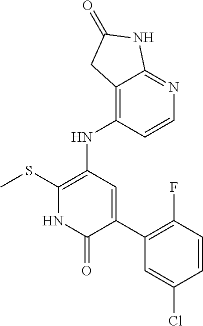 [SMILES: CSc1[nH]c(=O)c(-c2cc(Cl)ccc2F)cc1Nc1ccnc2c1CC(=O)N2] | 2.181 |
| 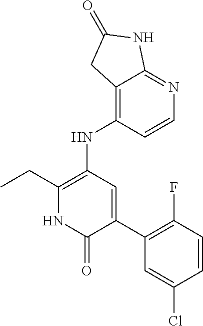 [SMILES: CCc1[nH]c(=O)c(-c2cc(Cl)ccc2F)cc1Nc1ccnc2c1CC(=O)N2] | 2.182 |
| 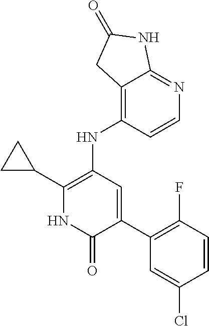 [SMILES: O=C1Cc2c(Nc3cc(-c4cc(Cl)ccc4F)c(=O)[nH]c3C3CC3)ccnc2N1] | 2.183 |
| 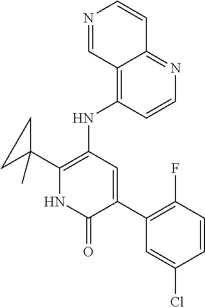 [SMILES: CC1(c2[nH]c(=O)c(-c3cc(Cl)ccc3F)cc2Nc2ccnc3ccncc23)CC1] | 2.184 |
| 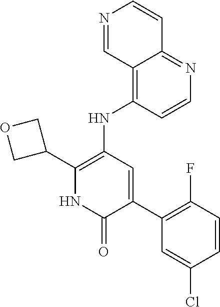 [SMILES: O=c1[nH]c(C2COC2)c(Nc2ccnc3ccncc23)cc1-c1cc(Cl)ccc1F] | 2.185 |
| 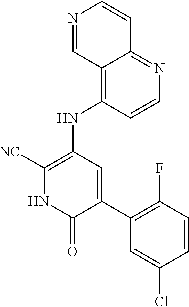 [SMILES: N#Cc1[nH]c(=O)c(-c2cc(Cl)ccc2F)cc1Nc1ccnc2ccncc12] | 2.186 |
| 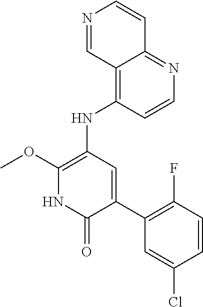 [SMILES: COc1[nH]c(=O)c(-c2cc(Cl)ccc2F)cc1Nc1ccnc2ccncc12] | 2.187 |
| 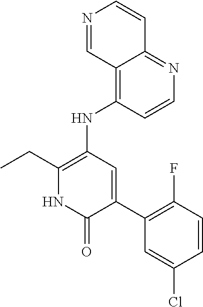 [SMILES: CCc1[nH]c(=O)c(-c2cc(Cl)ccc2F)cc1Nc1ccnc2ccncc12] | 2.188 |
| 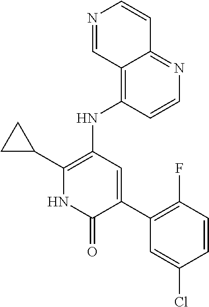 [SMILES: O=c1[nH]c(C2CC2)c(Nc2ccnc3ccncc23)cc1-c1cc(Cl)ccc1F] | 2.189 |
| 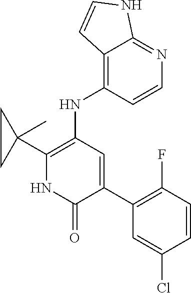 [SMILES: CC1(c2[nH]c(=O)c(-c3cc(Cl)ccc3F)cc2Nc2ccnc3[nH]ccc23)CC1] | 2.190 |
| 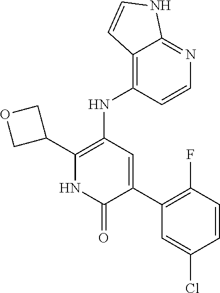 [SMILES: O=c1[nH]c(C2COC2)c(Nc2ccnc3[nH]ccc23)cc1-c1cc(Cl)ccc1F] | 2.191 |
| 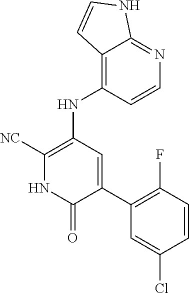 [SMILES: N#Cc1[nH]c(=O)c(-c2cc(Cl)ccc2F)cc1Nc1ccnc2[nH]ccc12] | 2.192 |
| 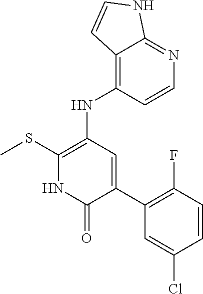 [SMILES: CSc1[nH]c(=O)c(-c2cc(Cl)ccc2F)cc1Nc1ccnc2[nH]ccc12] | 2.193 |
| 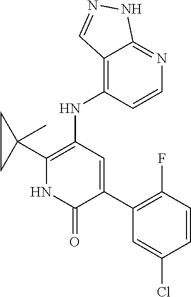 [SMILES: CC1(c2[nH]c(=O)c(-c3cc(Cl)ccc3F)cc2Nc2ccnc3[nH]ncc23)CC1] | 2.194 |
| 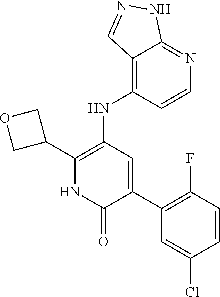 [SMILES: O=c1[nH]c(C2COC2)c(Nc2ccnc3[nH]ncc23)cc1-c1cc(Cl)ccc1F] | 2.195 |
| 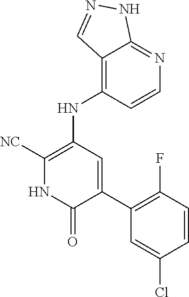 [SMILES: N#Cc1[nH]c(=O)c(-c2cc(Cl)ccc2F)cc1Nc1ccnc2[nH]ncc12] | 2.196 |
| 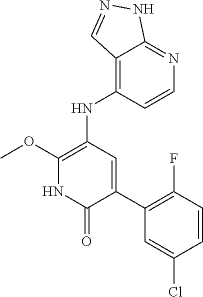 [SMILES: COc1[nH]c(=O)c(-c2cc(Cl)ccc2F)cc1Nc1ccnc2[nH]ncc12] | 2.197 |
| 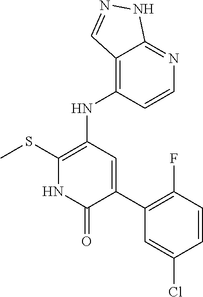 [SMILES: CSc1[nH]c(=O)c(-c2cc(Cl)ccc2F)cc1Nc1ccnc2[nH]ncc12] | 2.198 |
| 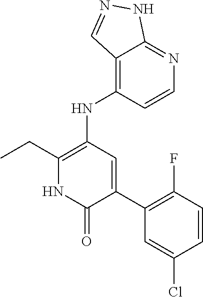 [SMILES: CCc1[nH]c(=O)c(-c2cc(Cl)ccc2F)cc1Nc1ccnc2[nH]ncc12] | 2.199 |
| 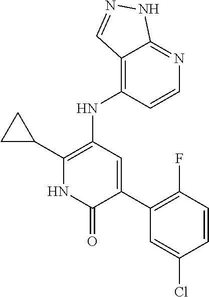 [SMILES: O=c1[nH]c(C2CC2)c(Nc2ccnc3[nH]ncc23)cc1-c1cc(Cl)ccc1F] | 2.200 |
| 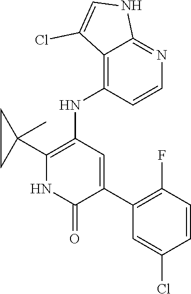 [SMILES: CC1(c2[nH]c(=O)c(-c3cc(Cl)ccc3F)cc2Nc2ccnc3[nH]cc(Cl)c23)CC1] | 2.201 |
| 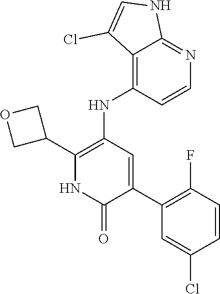 [SMILES: O=c1[nH]c(C2COC2)c(Nc2ccnc3[nH]cc(Cl)c23)cc1-c1cc(Cl)ccc1F] | 2.202 |
| 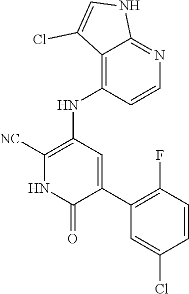 [SMILES: N#Cc1[nH]c(=O)c(-c2cc(Cl)ccc2F)cc1Nc1ccnc2[nH]cc(Cl)c12] | 2.203 |
| 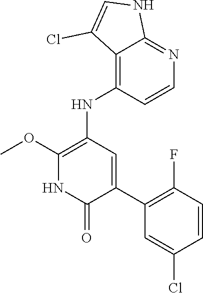 [SMILES: COc1[nH]c(=O)c(-c2cc(Cl)ccc2F)cc1Nc1ccnc2[nH]cc(Cl)c12] | 2.204 |
| 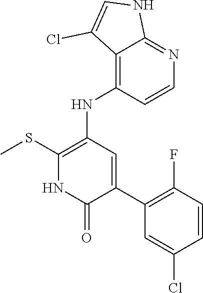 [SMILES: CSc1[nH]c(=O)c(-c2cc(Cl)ccc2F)cc1Nc1ccnc2[nH]cc(Cl)c12] | 2.205 |
| 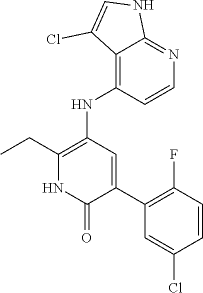 [SMILES: CCc1[nH]c(=O)c(-c2cc(Cl)ccc2F)cc1Nc1ccnc2[nH]cc(Cl)c12] | 2.206 |
| 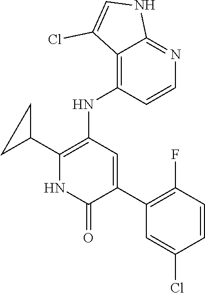 [SMILES: O=c1[nH]c(C2CC2)c(Nc2ccnc3[nH]cc(Cl)c23)cc1-c1cc(Cl)ccc1F] | 2.207 |
| 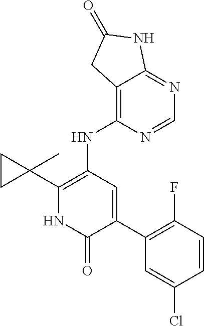 [SMILES: CC1(c2[nH]c(=O)c(-c3cc(Cl)ccc3F)cc2Nc2ncnc3c2CC(=O)N3)CC1] | 2.208 |
| 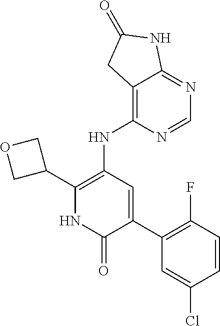 [SMILES: O=C1Cc2c(ncnc2Nc2cc(-c3cc(Cl)ccc3F)c(=O)[nH]c2C2COC2)N1] | 2.209 |
| 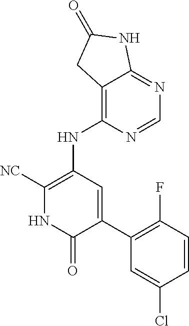 [SMILES: N#Cc1[nH]c(=O)c(-c2cc(Cl)ccc2F)cc1Nc1ncnc2c1CC(=O)N2] | 2.210 |
| 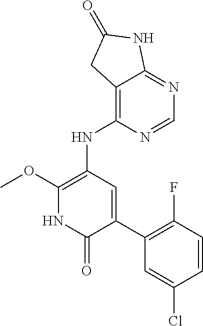 [SMILES: COc1[nH]c(=O)c(-c2cc(Cl)ccc2F)cc1Nc1ncnc2c1CC(=O)N2] | 2.211 |
| 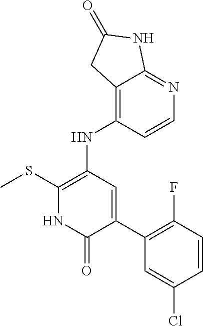 [SMILES: CSc1[nH]c(=O)c(-c2cc(Cl)ccc2F)cc1Nc1ccnc2c1CC(=O)N2] | 2.212 |
| 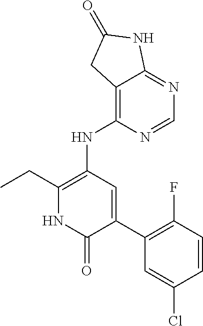 [SMILES: CCc1[nH]c(=O)c(-c2cc(Cl)ccc2F)cc1Nc1ncnc2c1CC(=O)N2] | 2.213 |
| 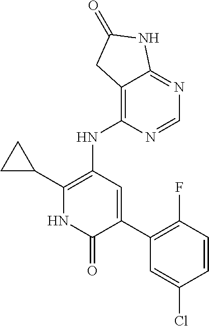 [SMILES: O=C1Cc2c(ncnc2Nc2cc(-c3cc(Cl)ccc3F)c(=O)[nH]c2C2CC2)N1] | 2.214 |
| 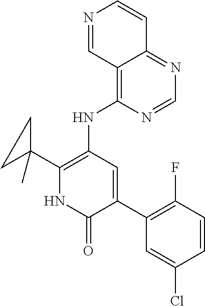 [SMILES: CC1(c2[nH]c(=O)c(-c3cc(Cl)ccc3F)cc2Nc2ncnc3ccncc23)CC1] | 2.215 |
| 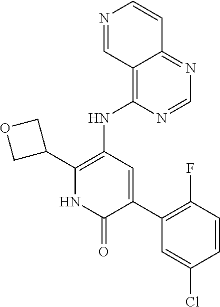 [SMILES: O=c1[nH]c(C2COC2)c(Nc2ncnc3ccncc23)cc1-c1cc(Cl)ccc1F] | 2.216 |
| 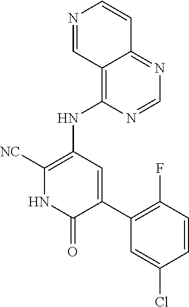 [SMILES: N#Cc1[nH]c(=O)c(-c2cc(Cl)ccc2F)cc1Nc1ncnc2ccncc12] | 2.217 |
| 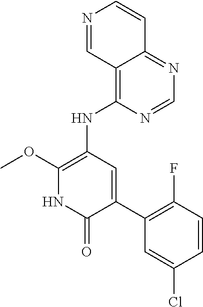 [SMILES: COc1[nH]c(=O)c(-c2cc(Cl)ccc2F)cc1Nc1ncnc2ccncc12] | 2.218 |
| 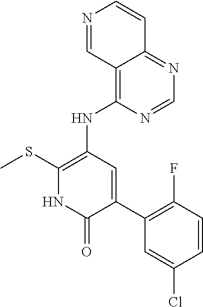 [SMILES: CSc1[nH]c(=O)c(-c2cc(Cl)ccc2F)cc1Nc1ncnc2ccncc12] | 2.219 |
| 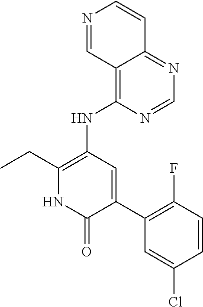 [SMILES: CCc1[nH]c(=O)c(-c2cc(Cl)ccc2F)cc1Nc1ncnc2ccncc12] | 2.220 |
| 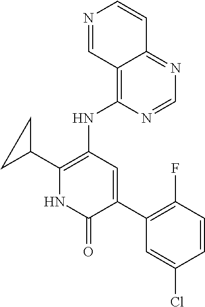 [SMILES: O=c1[nH]c(C2CC2)c(Nc2ncnc3ccncc23)cc1-c1cc(Cl)ccc1F] | 2.221 |
| 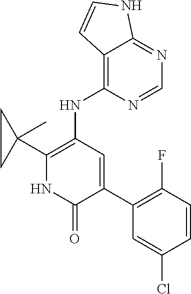 [SMILES: CC1(c2[nH]c(=O)c(-c3cc(Cl)ccc3F)cc2Nc2ncnc3[nH]ccc23)CC1] | 2.222 |
| 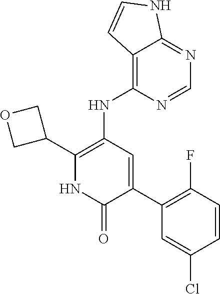 [SMILES: O=c1[nH]c(C2COC2)c(Nc2ncnc3[nH]ccc23)cc1-c1cc(Cl)ccc1F] | 2.223 |
| 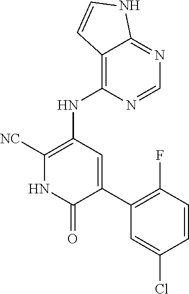 [SMILES: N#Cc1[nH]c(=O)c(-c2cc(Cl)ccc2F)cc1Nc1ncnc2[nH]ccc12] | 2.224 |
| 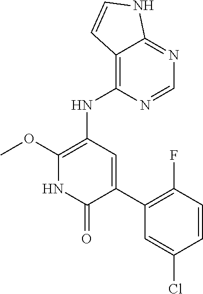 [SMILES: COc1[nH]c(=O)c(-c2cc(Cl)ccc2F)cc1Nc1ncnc2[nH]ccc12] | 2.225 |
| 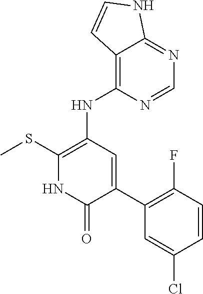 [SMILES: CSc1[nH]c(=O)c(-c2cc(Cl)ccc2F)cc1Nc1ncnc2[nH]ccc12] | 2.226 |
| 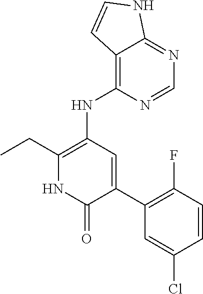 [SMILES: CCc1[nH]c(=O)c(-c2cc(Cl)ccc2F)cc1Nc1ncnc2[nH]ccc12] | 2.227 |
| 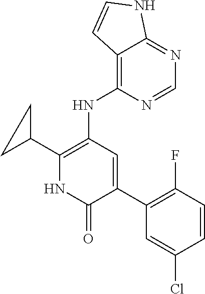 [SMILES: O=c1[nH]c(C2CC2)c(Nc2ncnc3[nH]ccc23)cc1-c1cc(Cl)ccc1F] | 2.228 |
| 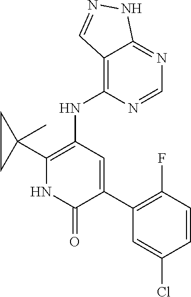 [SMILES: CC1(c2[nH]c(=O)c(-c3cc(Cl)ccc3F)cc2Nc2ncnc3[nH]ncc23)CC1] | 2.229 |
| 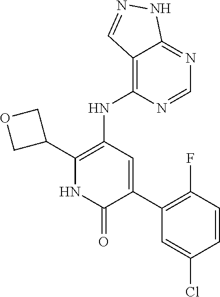 [SMILES: O=c1[nH]c(C2COC2)c(Nc2ncnc3[nH]ncc23)cc1-c1cc(Cl)ccc1F] | 2.230 |
| 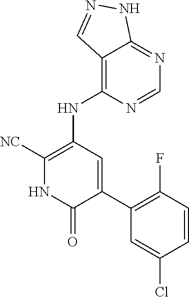 [SMILES: N#Cc1[nH]c(=O)c(-c2cc(Cl)ccc2F)cc1Nc1ncnc2[nH]ncc12] | 2.231 |
| 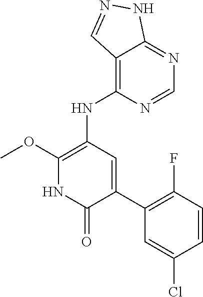 [SMILES: COc1[nH]c(=O)c(-c2cc(Cl)ccc2F)cc1Nc1ncnc2[nH]ncc12] | 2.232 |
| 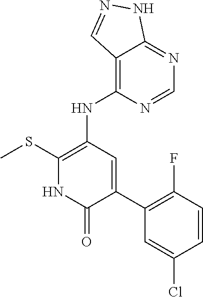 [SMILES: CSc1[nH]c(=O)c(-c2cc(Cl)ccc2F)cc1Nc1ncnc2[nH]ncc12] | 2.233 |
| 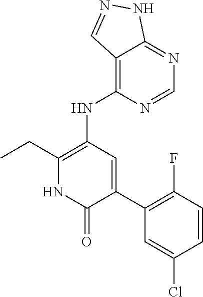 [SMILES: CCc1[nH]c(=O)c(-c2cc(Cl)ccc2F)cc1Nc1ncnc2[nH]ncc12] | 2.234 |
| 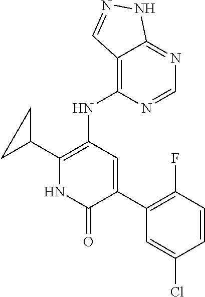 [SMILES: O=c1[nH]c(C2CC2)c(Nc2ncnc3[nH]ncc23)cc1-c1cc(Cl)ccc1F] | 2.235 |
| 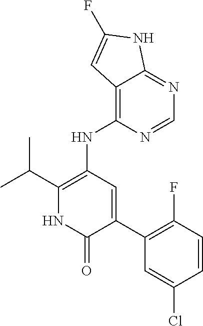 [SMILES: CC(C)c1[nH]c(=O)c(-c2cc(Cl)ccc2F)cc1Nc1ncnc2[nH]c(F)cc12] | 2.236 |
| 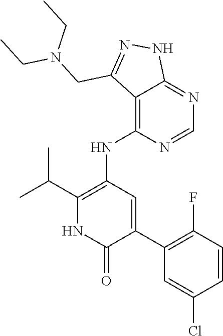 [SMILES: CCN(CC)Cc1n[nH]c2ncnc(Nc3cc(-c4cc(Cl)ccc4F)c(=O)[nH]c3C(C)C)c12] | 2.237 |
| 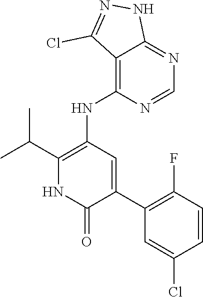 [SMILES: CC(C)c1[nH]c(=O)c(-c2cc(Cl)ccc2F)cc1Nc1ncnc2[nH]nc(Cl)c12] | 2.238 |
| 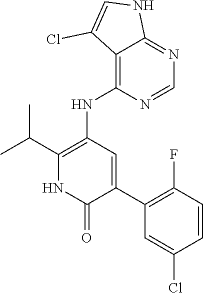 [SMILES: CC(C)c1[nH]c(=O)c(-c2cc(Cl)ccc2F)cc1Nc1ncnc2[nH]cc(Cl)c12] | 2.239 |
| 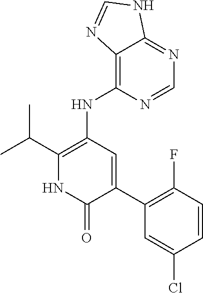 [SMILES: CC(C)c1[nH]c(=O)c(-c2cc(Cl)ccc2F)cc1Nc1ncnc2[nH]cnc12] | 2.240 |
| 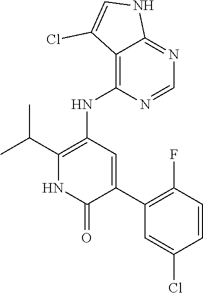 [SMILES: CC(C)c1[nH]c(=O)c(-c2cc(Cl)ccc2F)cc1Nc1ncnc2[nH]cc(Cl)c12] | 2.241 |
| 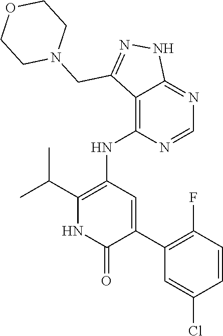 [SMILES: CC(C)c1[nH]c(=O)c(-c2cc(Cl)ccc2F)cc1Nc1ncnc2[nH]nc(CN3CCOCC3)c12] | 2.242 |
| 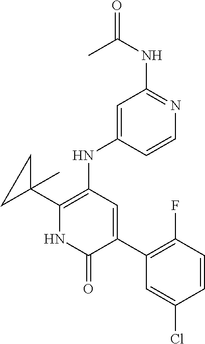 [SMILES: CC(=O)Nc1cc(Nc2cc(-c3cc(Cl)ccc3F)c(=O)[nH]c2C2(C)CC2)ccn1] | 2.243 |
| 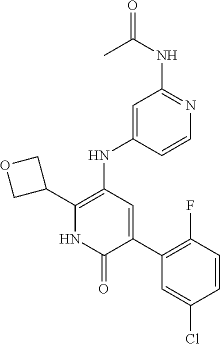 [SMILES: CC(=O)Nc1cc(Nc2cc(-c3cc(Cl)ccc3F)c(=O)[nH]c2C2COC2)ccn1] | 2.244 |
| 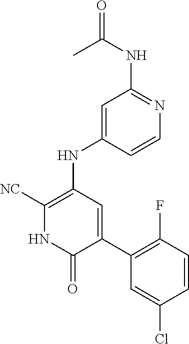 [SMILES: CC(=O)Nc1cc(Nc2cc(-c3cc(Cl)ccc3F)c(=O)[nH]c2C#N)ccn1] | 2.245 |
| 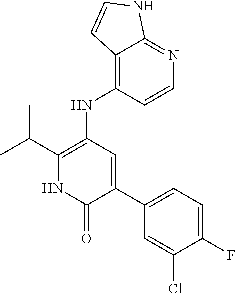 [SMILES: CC(C)c1[nH]c(=O)c(-c2ccc(F)c(Cl)c2)cc1Nc1ccnc2[nH]ccc12] | 2.246 |
| 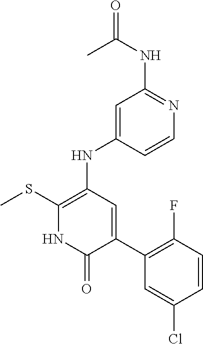 [SMILES: CSc1[nH]c(=O)c(-c2cc(Cl)ccc2F)cc1Nc1ccnc(NC(C)=O)c1] | 2.247 |
| 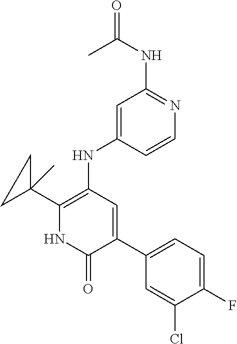 [SMILES: CC(=O)Nc1cc(Nc2cc(-c3ccc(F)c(Cl)c3)c(=O)[nH]c2C2(C)CC2)ccn1] | 2.248 |
| 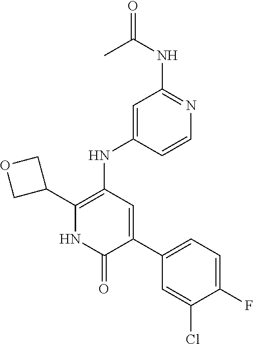 [SMILES: CC(=O)Nc1cc(Nc2cc(-c3ccc(F)c(Cl)c3)c(=O)[nH]c2C2COC2)ccn1] | 2.249 |
| 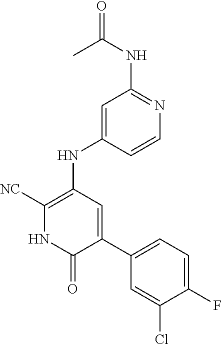 [SMILES: CC(=O)Nc1cc(Nc2cc(-c3ccc(F)c(Cl)c3)c(=O)[nH]c2C#N)ccn1] | 2.250 |
| 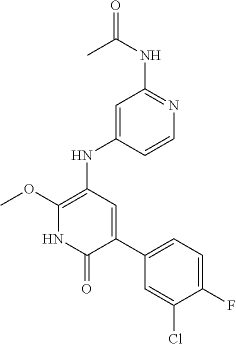 [SMILES: COc1[nH]c(=O)c(-c2ccc(F)c(Cl)c2)cc1Nc1ccnc(NC(C)=O)c1] | 2.251 |
| 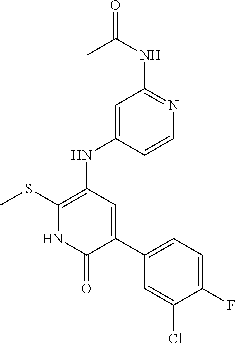 [SMILES: CSc1[nH]c(=O)c(-c2ccc(F)c(Cl)c2)cc1Nc1ccnc(NC(C)=O)c1] | 2.252 |
| 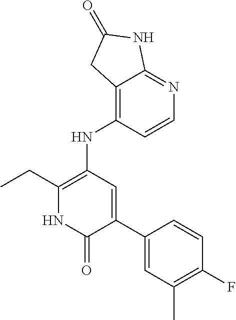 [SMILES: CCc1[nH]c(=O)c(-c2ccc(F)c(C)c2)cc1Nc1ccnc2c1CC(=O)N2] | 2.253 |
| 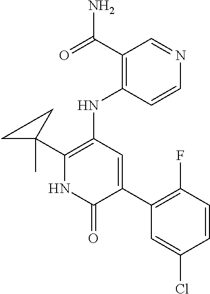 [SMILES: CC1(c2[nH]c(=O)c(-c3cc(Cl)ccc3F)cc2Nc2ccncc2C(N)=O)CC1] | 2.254 |
| 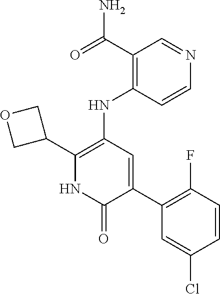 [SMILES: NC(=O)c1cnccc1Nc1cc(-c2cc(Cl)ccc2F)c(=O)[nH]c1C1COC1] | 2.255 |
| 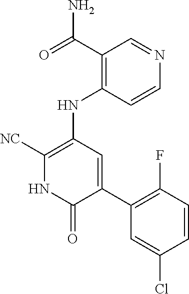 [SMILES: N#Cc1[nH]c(=O)c(-c2cc(Cl)ccc2F)cc1Nc1ccncc1C(N)=O] | 2.256 |
| 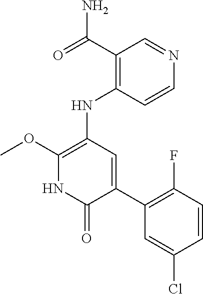 [SMILES: COc1[nH]c(=O)c(-c2cc(Cl)ccc2F)cc1Nc1ccncc1C(N)=O] | 2.257 |
| 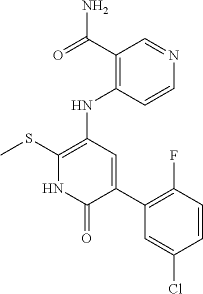 [SMILES: CSc1[nH]c(=O)c(-c2cc(Cl)ccc2F)cc1Nc1ccncc1C(N)=O] | 2.258 |
| 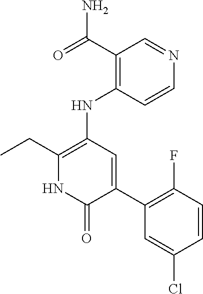 [SMILES: CCc1[nH]c(=O)c(-c2cc(Cl)ccc2F)cc1Nc1ccncc1C(N)=O] | 2.259 |
| 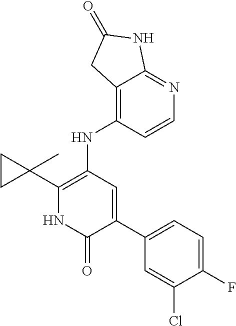 [SMILES: CC1(c2[nH]c(=O)c(-c3ccc(F)c(Cl)c3)cc2Nc2ccnc3c2CC(=O)N3)CC1] | 2.260 |
| 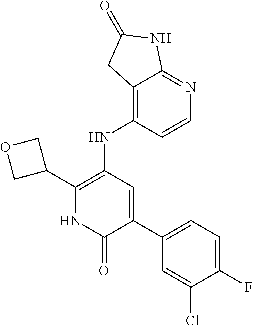 [SMILES: O=C1Cc2c(Nc3cc(-c4ccc(F)c(Cl)c4)c(=O)[nH]c3C3COC3)ccnc2N1] | 2.261 |
| 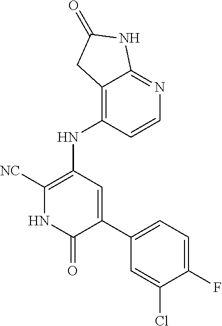 [SMILES: N#Cc1[nH]c(=O)c(-c2ccc(F)c(Cl)c2)cc1Nc1ccnc2c1CC(=O)N2] | 2.262 |
| 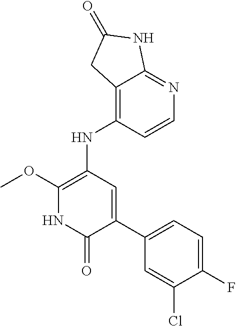 [SMILES: COc1[nH]c(=O)c(-c2ccc(F)c(Cl)c2)cc1Nc1ccnc2c1CC(=O)N2] | 2.263 |
| 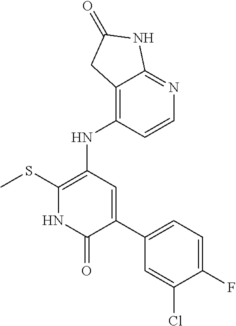 [SMILES: CSc1[nH]c(=O)c(-c2ccc(F)c(Cl)c2)cc1Nc1ccnc2c1CC(=O)N2] | 2.264 |
| 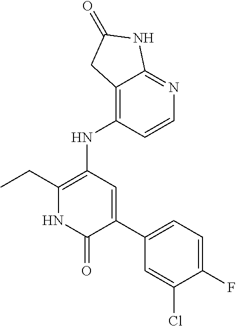 [SMILES: CCc1[nH]c(=O)c(-c2ccc(F)c(Cl)c2)cc1Nc1ccnc2c1CC(=O)N2] | 2.265 |
| 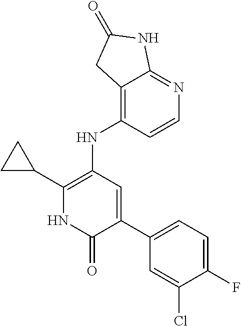 [SMILES: O=C1Cc2c(Nc3cc(-c4ccc(F)c(Cl)c4)c(=O)[nH]c3C3CC3)ccnc2N1] | 2.266 |
| 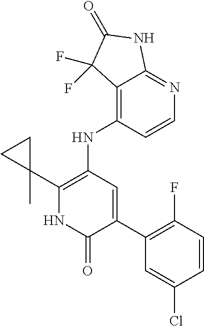 [SMILES: CC1(c2[nH]c(=O)c(-c3cc(Cl)ccc3F)cc2Nc2ccnc3c2C(F)(F)C(=O)N3)CC1] | 2.267 |
| 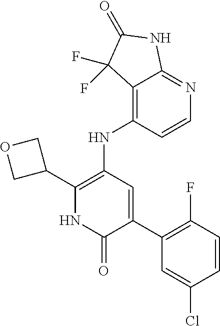 [SMILES: O=C1Nc2nccc(Nc3cc(-c4cc(Cl)ccc4F)c(=O)[nH]c3C3COC3)c2C1(F)F] | 2.268 |
| 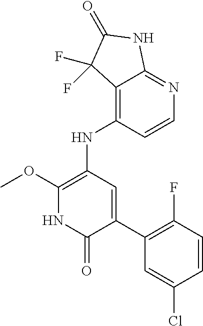 [SMILES: COc1[nH]c(=O)c(-c2cc(Cl)ccc2F)cc1Nc1ccnc2c1C(F)(F)C(=O)N2] | 2.269 |
| 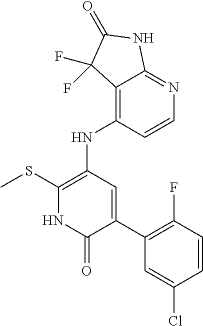 [SMILES: CSc1[nH]c(=O)c(-c2cc(Cl)ccc2F)cc1Nc1ccnc2c1C(F)(F)C(=O)N2] | 2.270 |
| 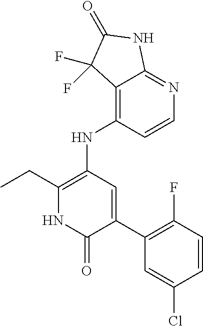 [SMILES: CCc1[nH]c(=O)c(-c2cc(Cl)ccc2F)cc1Nc1ccnc2c1C(F)(F)C(=O)N2] | 2.271 |
| 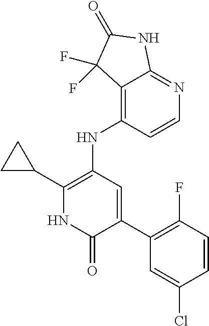 [SMILES: O=C1Nc2nccc(Nc3cc(-c4cc(Cl)ccc4F)c(=O)[nH]c3C3CC3)c2C1(F)F] | 2.272 |
| 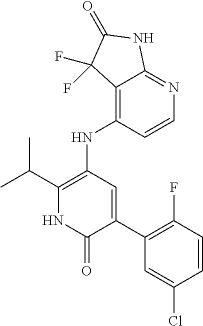 [SMILES: CC(C)c1[nH]c(=O)c(-c2cc(Cl)ccc2F)cc1Nc1ccnc2c1C(F)(F)C(=O)N2] | 2.273 |
| 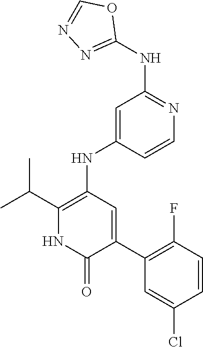 [SMILES: CC(C)c1[nH]c(=O)c(-c2cc(Cl)ccc2F)cc1Nc1ccnc(Nc2nnco2)c1] | 2.274 |
| 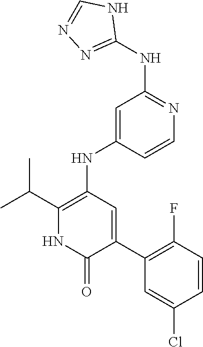 [SMILES: CC(C)c1[nH]c(=O)c(-c2cc(Cl)ccc2F)cc1Nc1ccnc(Nc2nnc[nH]2)c1] | 2.275 |
| 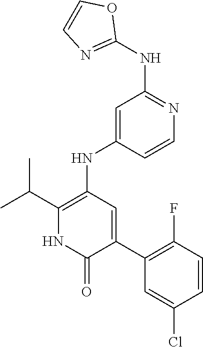 [SMILES: CC(C)c1[nH]c(=O)c(-c2cc(Cl)ccc2F)cc1Nc1ccnc(Nc2ncco2)c1] | 2.276 |
| 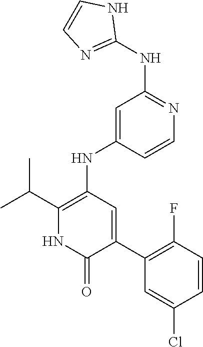 [SMILES: CC(C)c1[nH]c(=O)c(-c2cc(Cl)ccc2F)cc1Nc1ccnc(Nc2ncc[nH]2)c1] | 2.277 |
| 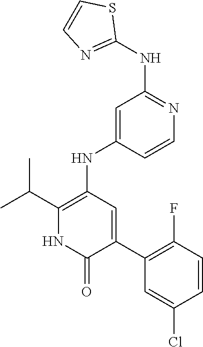 [SMILES: CC(C)c1[nH]c(=O)c(-c2cc(Cl)ccc2F)cc1Nc1ccnc(Nc2nccs2)c1] | 2.278 |
| 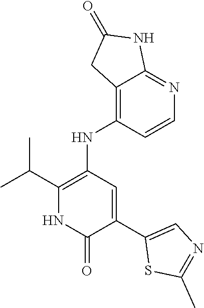 [SMILES: Cc1ncc(-c2cc(Nc3ccnc4c3CC(=O)N4)c(C(C)C)[nH]c2=O)s1] | 2.279 |
| 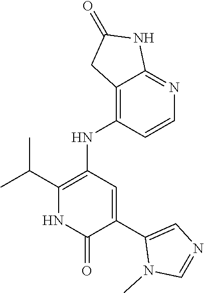 [SMILES: CC(C)c1[nH]c(=O)c(-c2cncn2C)cc1Nc1ccnc2c1CC(=O)N2] | 2.280 |
| 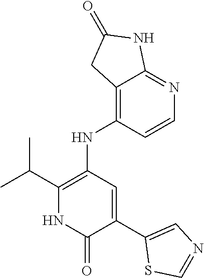 [SMILES: CC(C)c1[nH]c(=O)c(-c2cncs2)cc1Nc1ccnc2c1CC(=O)N2] | 2.281 |
| 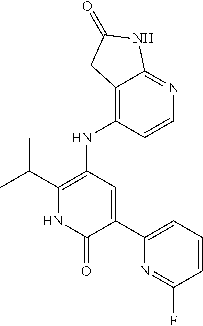 [SMILES: CC(C)c1[nH]c(=O)c(-c2cccc(F)n2)cc1Nc1ccnc2c1CC(=O)N2] | 2.282 |
| 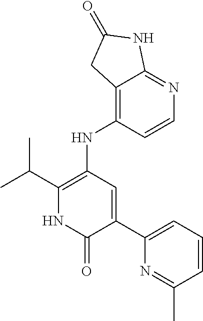 [SMILES: Cc1cccc(-c2cc(Nc3ccnc4c3CC(=O)N4)c(C(C)C)[nH]c2=O)n1] | 2.283 |
| 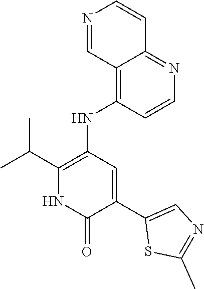 [SMILES: Cc1ncc(-c2cc(Nc3ccnc4ccncc34)c(C(C)C)[nH]c2=O)s1] | 2.284 |
| 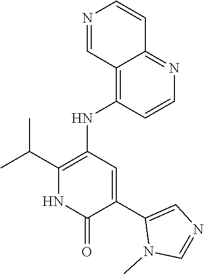 [SMILES: CC(C)c1[nH]c(=O)c(-c2cncn2C)cc1Nc1ccnc2ccncc12] | 2.285 |
| 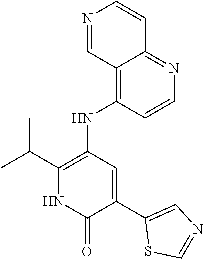 [SMILES: CC(C)c1[nH]c(=O)c(-c2cncs2)cc1Nc1ccnc2ccncc12] | 2.286 |
| 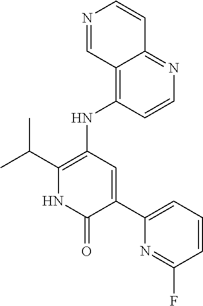 [SMILES: CC(C)c1[nH]c(=O)c(-c2cccc(F)n2)cc1Nc1ccnc2ccncc12] | 2.287 |
| 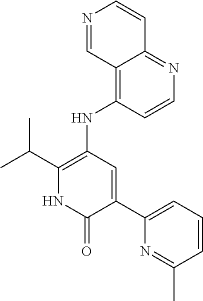 [SMILES: Cc1cccc(-c2cc(Nc3ccnc4ccncc34)c(C(C)C)[nH]c2=O)n1] | 2.288 |
| 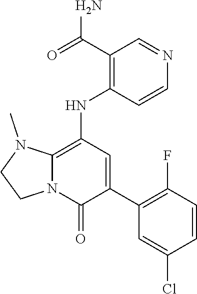 [SMILES: CN1CCn2c1c(Nc1ccncc1C(N)=O)cc(-c1cc(Cl)ccc1F)c2=O] | 2.289 |
| 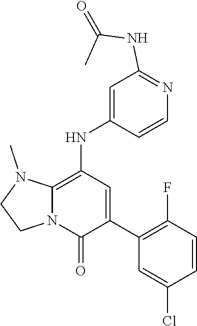 [SMILES: CC(=O)Nc1cc(Nc2cc(-c3cc(Cl)ccc3F)c(=O)n3c2N(C)CC3)ccn1] | 2.290 |
| 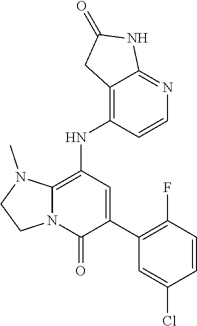 [SMILES: CN1CCn2c1c(Nc1ccnc3c1CC(=O)N3)cc(-c1cc(Cl)ccc1F)c2=O] | 2.291 |
| 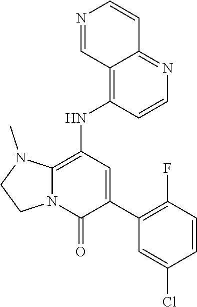 [SMILES: CN1CCn2c1c(Nc1ccnc3ccncc13)cc(-c1cc(Cl)ccc1F)c2=O] | 2.292 |
| 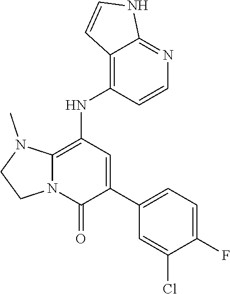 [SMILES: CN1CCn2c1c(Nc1ccnc3[nH]ccc13)cc(-c1ccc(F)c(Cl)c1)c2=O] | 2.293 |
| 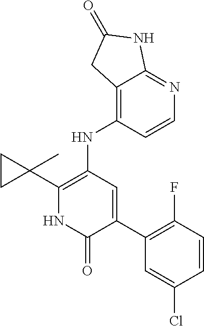 [SMILES: CC1(c2[nH]c(=O)c(-c3cc(Cl)ccc3F)cc2Nc2ccnc3c2CC(=O)N3)CC1] | 2.294 |
| 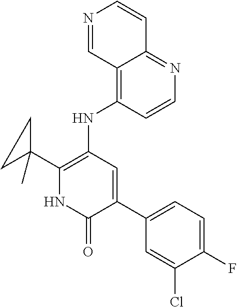 [SMILES: CC1(c2[nH]c(=O)c(-c3ccc(F)c(Cl)c3)cc2Nc2ccnc3ccncc23)CC1] | 2.295 |
| 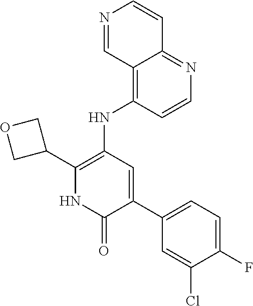 [SMILES: O=c1[nH]c(C2COC2)c(Nc2ccnc3ccncc23)cc1-c1ccc(F)c(Cl)c1] | 2.296 |
| 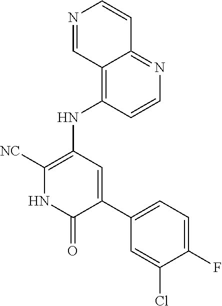 [SMILES: N#Cc1[nH]c(=O)c(-c2ccc(F)c(Cl)c2)cc1Nc1ccnc2ccncc12] | 2.297 |
| 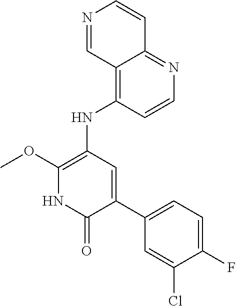 [SMILES: COc1[nH]c(=O)c(-c2ccc(F)c(Cl)c2)cc1Nc1ccnc2ccncc12] | 2.298 |
| 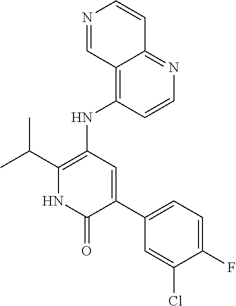 [SMILES: CC(C)c1[nH]c(=O)c(-c2ccc(F)c(Cl)c2)cc1Nc1ccnc2ccncc12] | 2.299 |
| 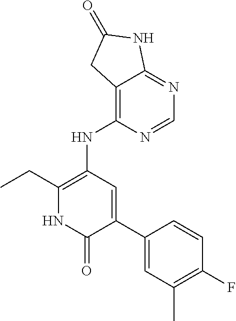 [SMILES: CCc1[nH]c(=O)c(-c2ccc(F)c(C)c2)cc1Nc1ncnc2c1CC(=O)N2] | 2.300 |
| 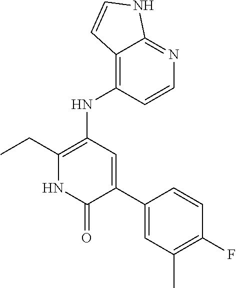 [SMILES: CCc1[nH]c(=O)c(-c2ccc(F)c(C)c2)cc1Nc1ccnc2[nH]ccc12] | 2.301 |
| 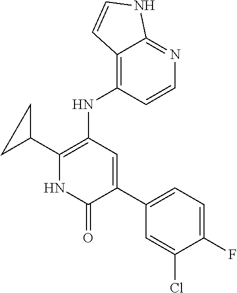 [SMILES: O=c1[nH]c(C2CC2)c(Nc2ccnc3[nH]ccc23)cc1-c1ccc(F)c(Cl)c1] | 2.302 |
| 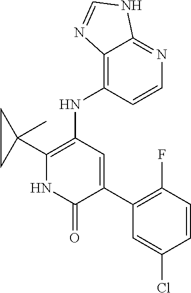 [SMILES: CC1(c2[nH]c(=O)c(-c3cc(Cl)ccc3F)cc2Nc2ccnc3[nH]cnc23)CC1] | 2.303 |
| 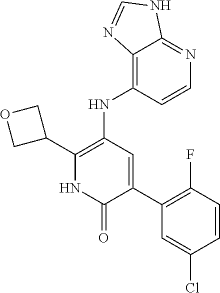 [SMILES: O=c1[nH]c(C2COC2)c(Nc2ccnc3[nH]cnc23)cc1-c1cc(Cl)ccc1F] | 2.304 |
| 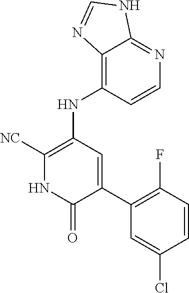 [SMILES: N#Cc1[nH]c(=O)c(-c2cc(Cl)ccc2F)cc1Nc1ccnc2[nH]cnc12] | 2.305 |
| 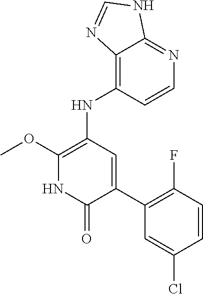 [SMILES: COc1[nH]c(=O)c(-c2cc(Cl)ccc2F)cc1Nc1ccnc2[nH]cnc12] | 2.306 |
| 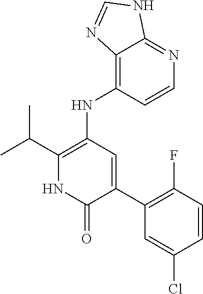 [SMILES: CC(C)c1[nH]c(=O)c(-c2cc(Cl)ccc2F)cc1Nc1ccnc2[nH]cnc12] | 2.307 |
| 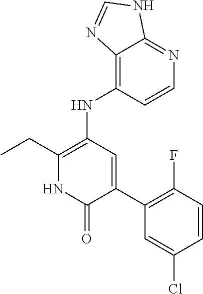 [SMILES: CCc1[nH]c(=O)c(-c2cc(Cl)ccc2F)cc1Nc1ccnc2[nH]cnc12] | 2.308 |
| 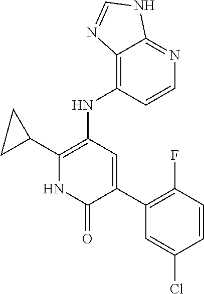 [SMILES: O=c1[nH]c(C2CC2)c(Nc2ccnc3[nH]cnc23)cc1-c1cc(Cl)ccc1F] | 2.309 |
| 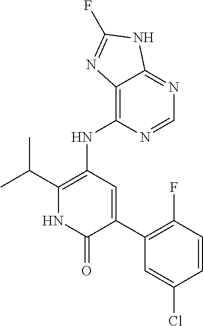 [SMILES: CC(C)c1[nH]c(=O)c(-c2cc(Cl)ccc2F)cc1Nc1ncnc2[nH]c(F)nc12] | 2.310 |
| 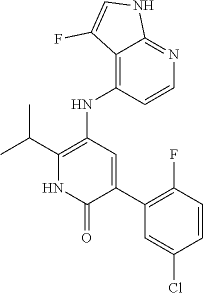 [SMILES: CC(C)c1[nH]c(=O)c(-c2cc(Cl)ccc2F)cc1Nc1ccnc2[nH]cc(F)c12] | 2.311 |
| 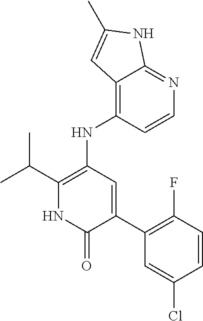 [SMILES: Cc1cc2c(Nc3cc(-c4cc(Cl)ccc4F)c(=O)[nH]c3C(C)C)ccnc2[nH]1] | 2.312 |
| 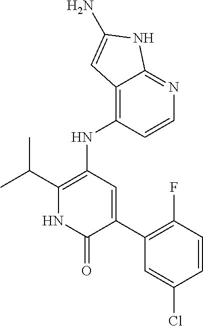 [SMILES: CC(C)c1[nH]c(=O)c(-c2cc(Cl)ccc2F)cc1Nc1ccnc2[nH]c(N)cc12] | 2.313 |
| 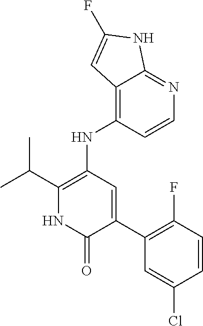 [SMILES: CC(C)c1[nH]c(=O)c(-c2cc(Cl)ccc2F)cc1Nc1ccnc2[nH]c(F)cc12] | 2.314 |
| 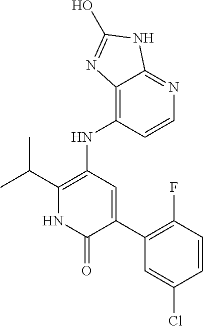 [SMILES: CC(C)c1[nH]c(=O)c(-c2cc(Cl)ccc2F)cc1Nc1ccnc2[nH]c(O)nc12] | 2.315 |
| 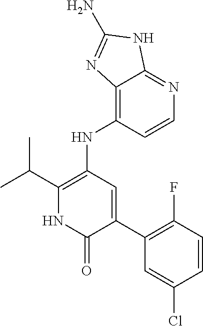 [SMILES: CC(C)c1[nH]c(=O)c(-c2cc(Cl)ccc2F)cc1Nc1ccnc2[nH]c(N)nc12] | 2.316 |
| 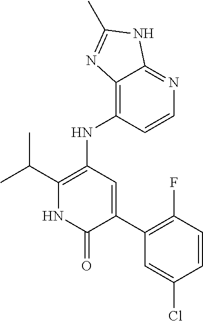 [SMILES: Cc1nc2c(Nc3cc(-c4cc(Cl)ccc4F)c(=O)[nH]c3C(C)C)ccnc2[nH]1] | 2.317 |
| 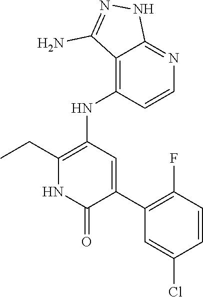 [SMILES: CCc1[nH]c(=O)c(-c2cc(Cl)ccc2F)cc1Nc1ccnc2[nH]nc(N)c12] | 2.318 |
| 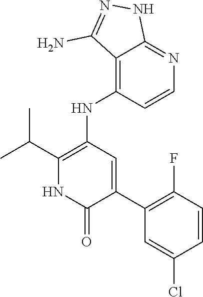 [SMILES: CC(C)c1[nH]c(=O)c(-c2cc(Cl)ccc2F)cc1Nc1ccnc2[nH]nc(N)c12] | 2.319 |
| 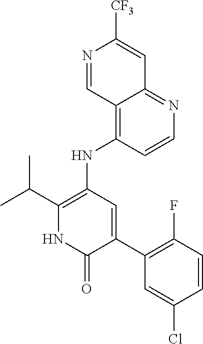 [SMILES: CC(C)c1[nH]c(=O)c(-c2cc(Cl)ccc2F)cc1Nc1ccnc2cc(C(F)(F)F)ncc12] | 2.320 |
| 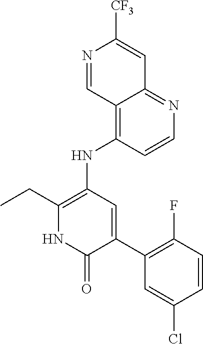 [SMILES: CCc1[nH]c(=O)c(-c2cc(Cl)ccc2F)cc1Nc1ccnc2cc(C(F)(F)F)ncc12] | 2.321 |
| 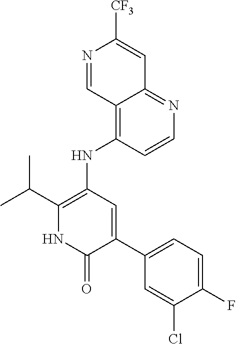 [SMILES: CC(C)c1[nH]c(=O)c(-c2ccc(F)c(Cl)c2)cc1Nc1ccnc2cc(C(F)(F)F)ncc12] | 2.322 |
| 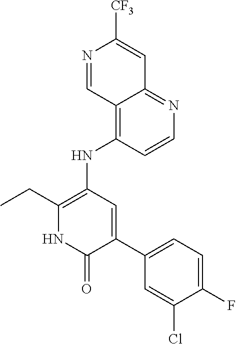 [SMILES: CCc1[nH]c(=O)c(-c2ccc(F)c(Cl)c2)cc1Nc1ccnc2cc(C(F)(F)F)ncc12] | 2.323 |
| 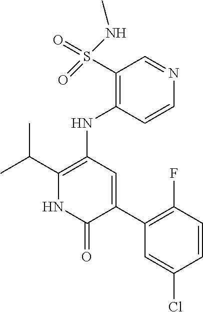 [SMILES: CNS(=O)(=O)c1cnccc1Nc1cc(-c2cc(Cl)ccc2F)c(=O)[nH]c1C(C)C] | 2.324 |
| 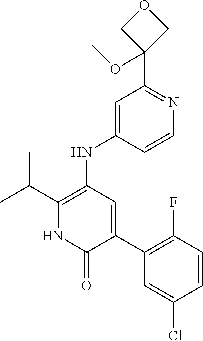 [SMILES: COC1(c2cc(Nc3cc(-c4cc(Cl)ccc4F)c(=O)[nH]c3C(C)C)ccn2)COC1] | 2.325 |
| 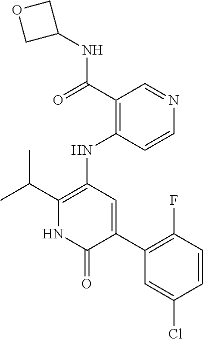 [SMILES: CC(C)c1[nH]c(=O)c(-c2cc(Cl)ccc2F)cc1Nc1ccncc1C(=O)NC1COC1] | 2.326 |
| 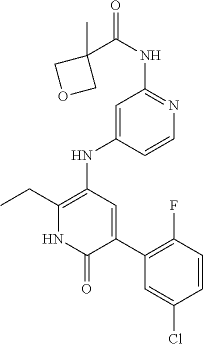 [SMILES: CCc1[nH]c(=O)c(-c2cc(Cl)ccc2F)cc1Nc1ccnc(NC(=O)C2(C)COC2)c1] | 2.327 |
| 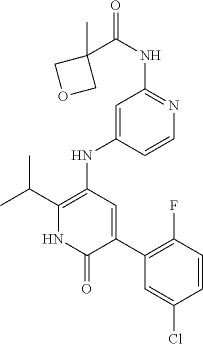 [SMILES: CC(C)c1[nH]c(=O)c(-c2cc(Cl)ccc2F)cc1Nc1ccnc(NC(=O)C2(C)COC2)c1] | 2.328 |
| 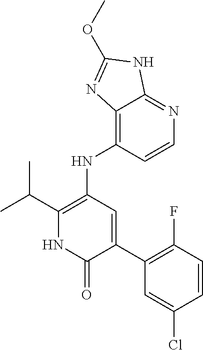 [SMILES: COc1nc2c(Nc3cc(-c4cc(Cl)ccc4F)c(=O)[nH]c3C(C)C)ccnc2[nH]1] | 2.329 |
| 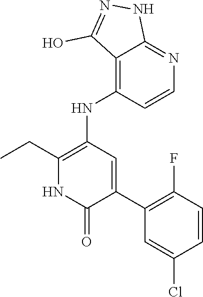 [SMILES: CCc1[nH]c(=O)c(-c2cc(Cl)ccc2F)cc1Nc1ccnc2[nH]nc(O)c12] | 2.330 |
| 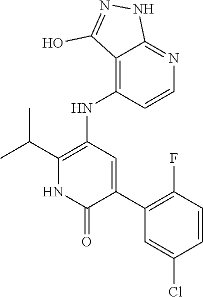 [SMILES: CC(C)c1[nH]c(=O)c(-c2cc(Cl)ccc2F)cc1Nc1ccnc2[nH]nc(O)c12] | 2.331 |
| 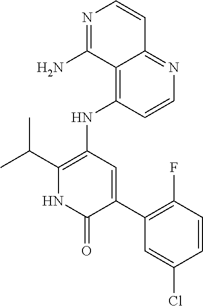 [SMILES: CC(C)c1[nH]c(=O)c(-c2cc(Cl)ccc2F)cc1Nc1ccnc2ccnc(N)c12] | 2.332 |
| 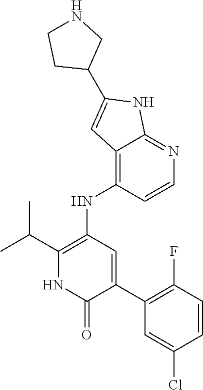 [SMILES: CC(C)c1[nH]c(=O)c(-c2cc(Cl)ccc2F)cc1Nc1ccnc2[nH]c(C3CCNC3)cc12] | 2.333 |
| 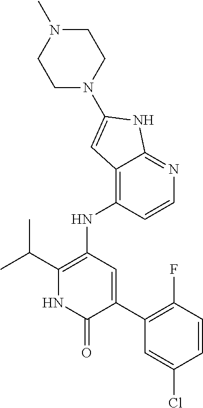 [SMILES: CC(C)c1[nH]c(=O)c(-c2cc(Cl)ccc2F)cc1Nc1ccnc2[nH]c(N3CCN(C)CC3)cc12] | 2.334 |
| 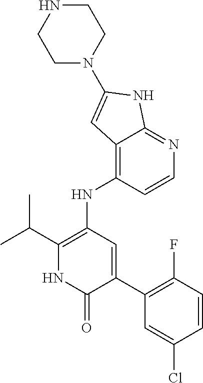 [SMILES: CC(C)c1[nH]c(=O)c(-c2cc(Cl)ccc2F)cc1Nc1ccnc2[nH]c(N3CCNCC3)cc12] | 2.335 |
| 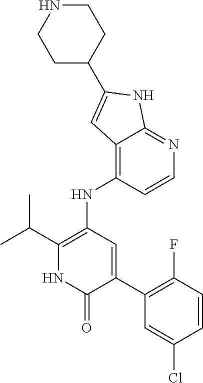 [SMILES: CC(C)c1[nH]c(=O)c(-c2cc(Cl)ccc2F)cc1Nc1ccnc2[nH]c(C3CCNCC3)cc12] | 2.336 |
| 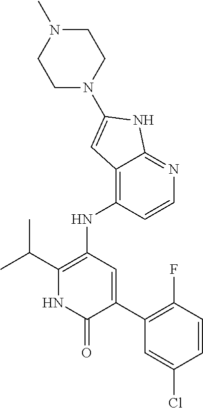 [SMILES: CC(C)c1[nH]c(=O)c(-c2cc(Cl)ccc2F)cc1Nc1ccnc2[nH]c(N3CCN(C)CC3)cc12] | 2.337 |
| 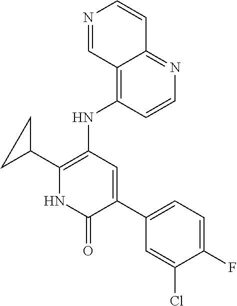 [SMILES: O=c1[nH]c(C2CC2)c(Nc2ccnc3ccncc23)cc1-c1ccc(F)c(Cl)c1] | 2.338 |
| 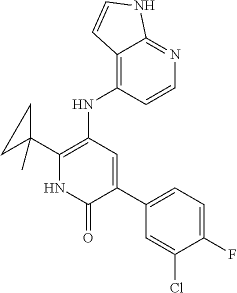 [SMILES: CC1(c2[nH]c(=O)c(-c3ccc(F)c(Cl)c3)cc2Nc2ccnc3[nH]ccc23)CC1] | 2.339 |
| 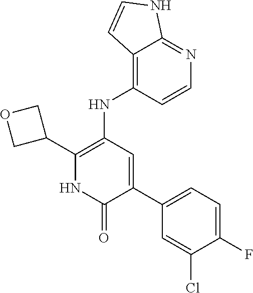 [SMILES: O=c1[nH]c(C2COC2)c(Nc2ccnc3[nH]ccc23)cc1-c1ccc(F)c(Cl)c1] | 2.340 |
| 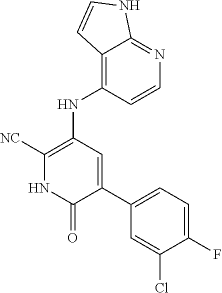 [SMILES: N#Cc1[nH]c(=O)c(-c2ccc(F)c(Cl)c2)cc1Nc1ccnc2[nH]ccc12] | 2.341 |
| 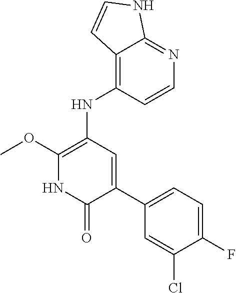 [SMILES: COc1[nH]c(=O)c(-c2ccc(F)c(Cl)c2)cc1Nc1ccnc2[nH]ccc12] | 2.342 |
| 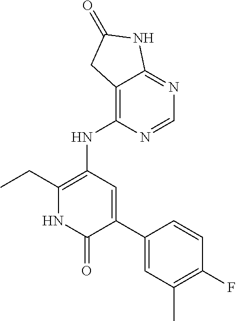 [SMILES: CCc1[nH]c(=O)c(-c2ccc(F)c(C)c2)cc1Nc1ncnc2c1CC(=O)N2] | 2.343 |
| 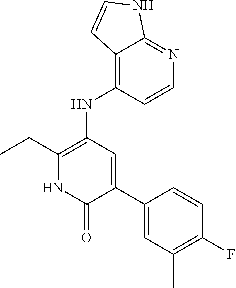 [SMILES: CCc1[nH]c(=O)c(-c2ccc(F)c(C)c2)cc1Nc1ccnc2[nH]ccc12] | 2.344 |
| 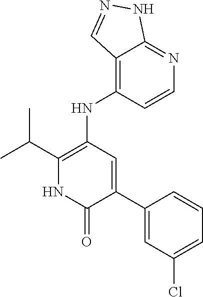 [SMILES: CC(C)c1[nH]c(=O)c(-c2cccc(Cl)c2)cc1Nc1ccnc2[nH]ncc12] | 2.345 |
| 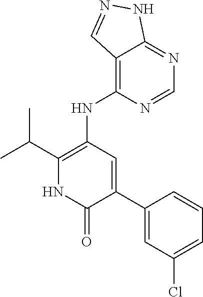 [SMILES: CC(C)c1[nH]c(=O)c(-c2cccc(Cl)c2)cc1Nc1ncnc2[nH]ncc12] | 2.346 |
| 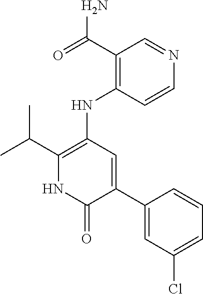 [SMILES: CC(C)c1[nH]c(=O)c(-c2cccc(Cl)c2)cc1Nc1ccncc1C(N)=O] | 2.347 |
| 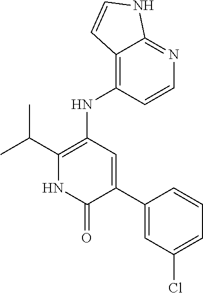 [SMILES: CC(C)c1[nH]c(=O)c(-c2cccc(Cl)c2)cc1Nc1ccnc2[nH]ccc12] | 2.348 |
| 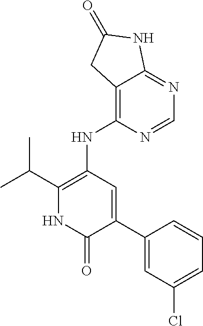 [SMILES: CC(C)c1[nH]c(=O)c(-c2cccc(Cl)c2)cc1Nc1ncnc2c1CC(=O)N2] | 2.349 |
| 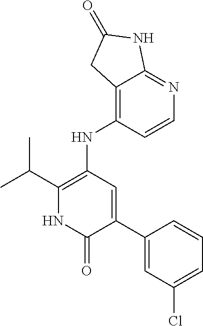 [SMILES: CC(C)c1[nH]c(=O)c(-c2cccc(Cl)c2)cc1Nc1ccnc2c1CC(=O)N2] | 2.350 |
| 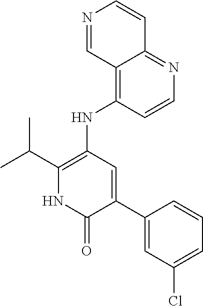 [SMILES: CC(C)c1[nH]c(=O)c(-c2cccc(Cl)c2)cc1Nc1ccnc2ccncc12] | 2.351 |
| 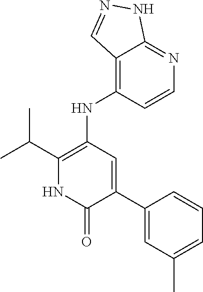 [SMILES: Cc1cccc(-c2cc(Nc3ccnc4[nH]ncc34)c(C(C)C)[nH]c2=O)c1] | 2.352 |
| 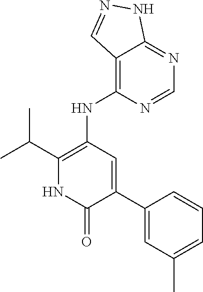 [SMILES: Cc1cccc(-c2cc(Nc3ncnc4[nH]ncc34)c(C(C)C)[nH]c2=O)c1] | 2.353 |
| 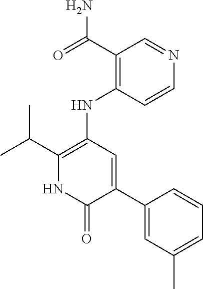 [SMILES: Cc1cccc(-c2cc(Nc3ccncc3C(N)=O)c(C(C)C)[nH]c2=O)c1] | 2.354 |
| 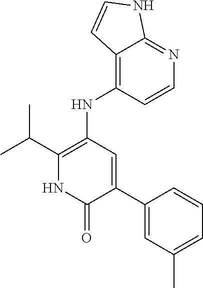 [SMILES: Cc1cccc(-c2cc(Nc3ccnc4[nH]ccc34)c(C(C)C)[nH]c2=O)c1] | 2.355 |
| 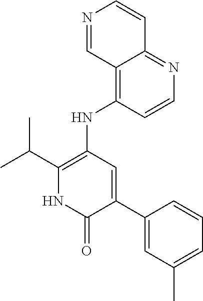 [SMILES: Cc1cccc(-c2cc(Nc3ccnc4ccncc34)c(C(C)C)[nH]c2=O)c1] | 2.356 |
| 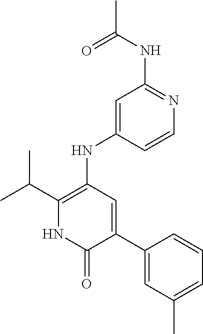 [SMILES: CC(=O)Nc1cc(Nc2cc(-c3cccc(C)c3)c(=O)[nH]c2C(C)C)ccn1] | 2.357 |
| 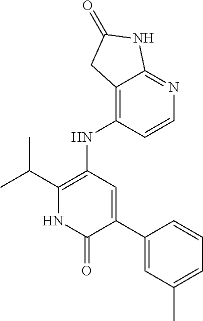 [SMILES: Cc1cccc(-c2cc(Nc3ccnc4c3CC(=O)N4)c(C(C)C)[nH]c2=O)c1] | 2.358 |
| 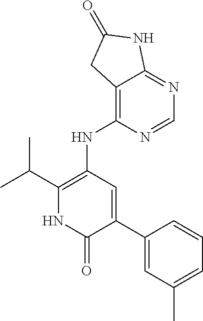 [SMILES: Cc1cccc(-c2cc(Nc3ncnc4c3CC(=O)N4)c(C(C)C)[nH]c2=O)c1] | 2.359 |
| 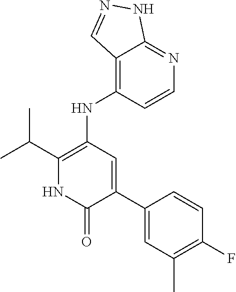 [SMILES: Cc1cc(-c2cc(Nc3ccnc4[nH]ncc34)c(C(C)C)[nH]c2=O)ccc1F] | 2.360 |
| 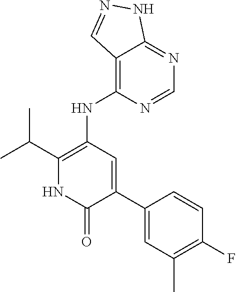 [SMILES: Cc1cc(-c2cc(Nc3ncnc4[nH]ncc34)c(C(C)C)[nH]c2=O)ccc1F] | 2.361 |
| 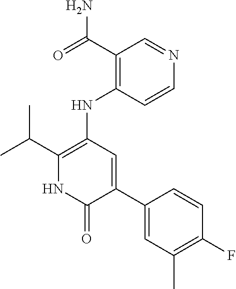 [SMILES: Cc1cc(-c2cc(Nc3ccncc3C(N)=O)c(C(C)C)[nH]c2=O)ccc1F] | 2.362 |
| 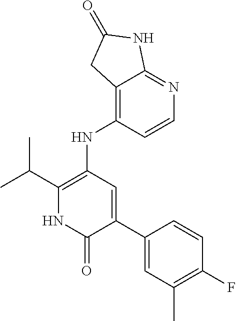 [SMILES: Cc1cc(-c2cc(Nc3ccnc4c3CC(=O)N4)c(C(C)C)[nH]c2=O)ccc1F] | 2.363 |
| 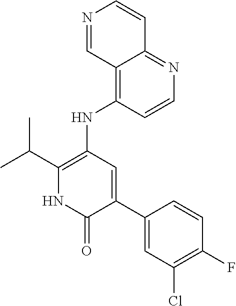 [SMILES: CC(C)c1[nH]c(=O)c(-c2ccc(F)c(Cl)c2)cc1Nc1ccnc2ccncc12] | 2.364 |
| 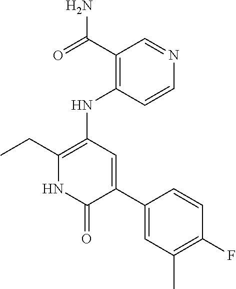 [SMILES: CCc1[nH]c(=O)c(-c2ccc(F)c(C)c2)cc1Nc1ccncc1C(N)=O] | 2.365 |
| 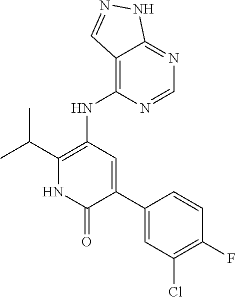 [SMILES: CC(C)c1[nH]c(=O)c(-c2ccc(F)c(Cl)c2)cc1Nc1ncnc2[nH]ncc12] | 2.366 |
| 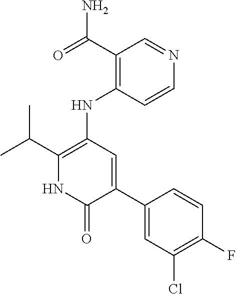 [SMILES: CC(C)c1[nH]c(=O)c(-c2ccc(F)c(Cl)c2)cc1Nc1ccncc1C(N)=O] | 2.367 |
| 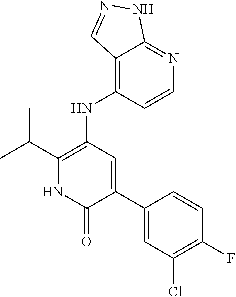 [SMILES: CC(C)c1[nH]c(=O)c(-c2ccc(F)c(Cl)c2)cc1Nc1ccnc2[nH]ncc12] | 2.368 |
| 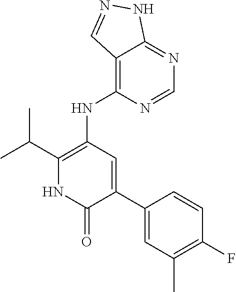 [SMILES: Cc1cc(-c2cc(Nc3ncnc4[nH]ncc34)c(C(C)C)[nH]c2=O)ccc1F] | 2.369 |
| 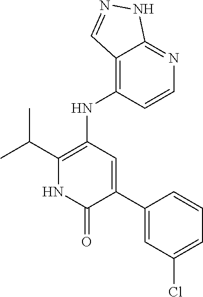 [SMILES: CC(C)c1[nH]c(=O)c(-c2cccc(Cl)c2)cc1Nc1ccnc2[nH]ncc12] | 2.370 |
| 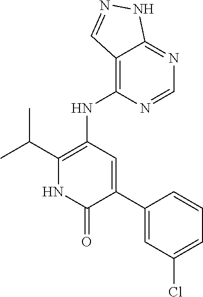 [SMILES: CC(C)c1[nH]c(=O)c(-c2cccc(Cl)c2)cc1Nc1ncnc2[nH]ncc12] | 2.371 |
| 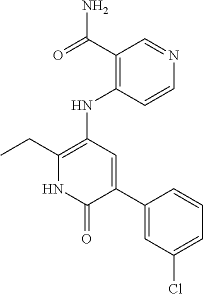 [SMILES: CCc1[nH]c(=O)c(-c2cccc(Cl)c2)cc1Nc1ccncc1C(N)=O] | 2.372 |
| 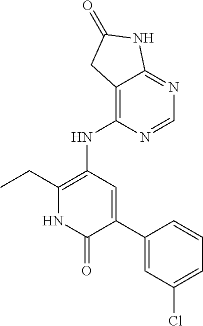 [SMILES: CCc1[nH]c(=O)c(-c2cccc(Cl)c2)cc1Nc1ncnc2c1CC(=O)N2] | 2.373 |
| 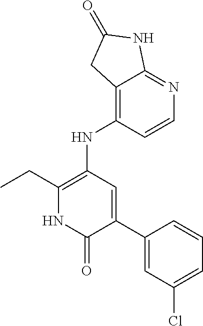 [SMILES: CCc1[nH]c(=O)c(-c2cccc(Cl)c2)cc1Nc1ccnc2c1CC(=O)N2] | 2.374 |
| 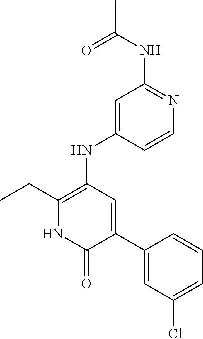 [SMILES: CCc1[nH]c(=O)c(-c2cccc(Cl)c2)cc1Nc1ccnc(NC(C)=O)c1] | 2.375 |
| 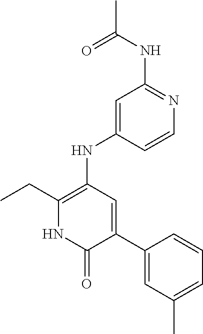 [SMILES: CCc1[nH]c(=O)c(-c2cccc(C)c2)cc1Nc1ccnc(NC(C)=O)c1] | 2.376 |
| 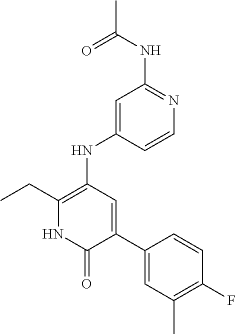 [SMILES: CCc1[nH]c(=O)c(-c2ccc(F)c(C)c2)cc1Nc1ccnc(NC(C)=O)c1] | 2.377 |
| 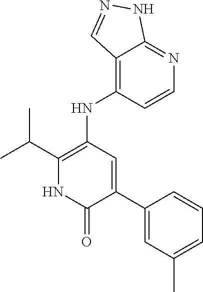 [SMILES: Cc1cccc(-c2cc(Nc3ccnc4[nH]ncc34)c(C(C)C)[nH]c2=O)c1] | 2.378 |
| 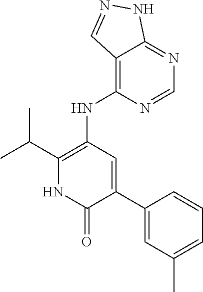 [SMILES: Cc1cccc(-c2cc(Nc3ncnc4[nH]ncc34)c(C(C)C)[nH]c2=O)c1] | 2.379 |
| 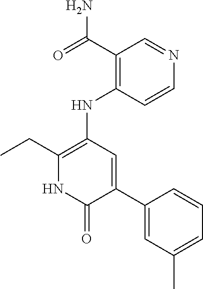 [SMILES: CCc1[nH]c(=O)c(-c2cccc(C)c2)cc1Nc1ccncc1C(N)=O] | 2.380 |
| 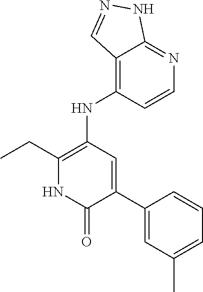 [SMILES: CCc1[nH]c(=O)c(-c2cccc(C)c2)cc1Nc1ccnc2[nH]ncc12] | 2.381 |
| 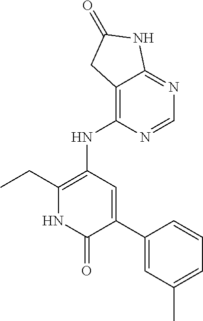 [SMILES: CCc1[nH]c(=O)c(-c2cccc(C)c2)cc1Nc1ncnc2c1CC(=O)N2] | 2.382 |
| 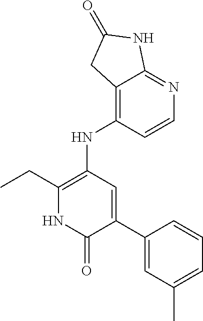 [SMILES: CCc1[nH]c(=O)c(-c2cccc(C)c2)cc1Nc1ccnc2c1CC(=O)N2] | 2.383 |
| 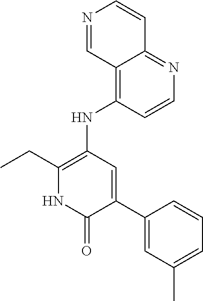 [SMILES: CCc1[nH]c(=O)c(-c2cccc(C)c2)cc1Nc1ccnc2ccncc12] | 2.384 |
| 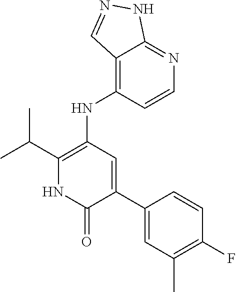 [SMILES: Cc1cc(-c2cc(Nc3ccnc4[nH]ncc34)c(C(C)C)[nH]c2=O)ccc1F] | 2.385 |
| 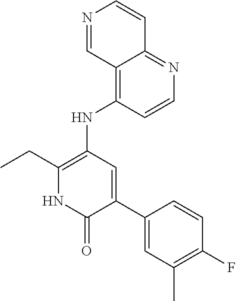 [SMILES: CCc1[nH]c(=O)c(-c2ccc(F)c(C)c2)cc1Nc1ccnc2ccncc12] | 2.386 |

In some embodiments, provided herein are compounds described in Table 1 and Table 2, or a tautomer thereof, or a salt of any of the foregoing, and uses thereof.

The embodiments and variations described herein are suitable for compounds of any formulae detailed herein, where applicable.

Representative examples of compounds detailed herein, including intermediates and final compounds according to the present disclosure are depicted herein. It is understood that in one aspect, any of the compounds may be used in the methods detailed herein, including, where applicable, intermediate compounds that may be isolated and administered to an individual.

The compounds depicted herein may be present as salts even if salts are not depicted and it is understood that the present disclosure embraces all salts and solvates of the compounds depicted here, as well as the non-salt and non-solvate form of the compound, as is well understood by the skilled artisan. In some embodiments, the salts of the compounds provided herein are pharmaceutically acceptable salts. Where one or more tertiary amine moiety is present in the compound, the N-oxides are also provided and described.

Where tautomeric forms may be present for any of the compounds described herein, each and every tautomeric form is intended even though only one or some of the tautomeric forms may be explicitly depicted. The tautomeric forms specifically depicted may or may not be the predominant forms in solution or when used according to the methods described herein.

The present disclosure also includes any or all of the stereochemical forms, including any enantiomeric or diastereomeric forms of the compounds described. All forms of the compounds are also embraced by the invention, such as crystalline or non-crystalline forms of the compounds. Compositions comprising a compound of the invention are also intended, such as a composition of substantially pure compound, including a specific stereochemical form thereof, or a composition comprising mixtures of compounds of the invention in any ratio, including two or more stereochemical forms, such as in a racemic or non-racemic mixture.

The invention also intends isotopically-labeled and/or isotopically-enriched forms of compounds described herein. The compounds herein may contain unnatural proportions of atomic isotopes at one or more of the atoms that constitute such compounds. In some embodiments, the compound is isotopically-labeled, such as an isotopically-labeled compound of the formula (I) or variations thereof described herein, where a fraction of one or more atoms are replaced by an isotope of the same element. Exemplary isotopes that can be incorporated into compounds of the invention include isotopes of hydrogen, carbon, nitrogen, oxygen, phosphorus, sulfur, chlorine, such as ²H, ³H, ¹¹C, ¹³C, ¹⁴C ¹³N, ¹⁵O, ¹⁷O, ³²P, ³⁵S, ¹⁸F, ³⁶Cl. Certain isotope labeled compounds (e.g. ³H and ¹⁴C) are useful in compound or substrate tissue distribution studies. Incorporation of heavier isotopes such as deuterium (²H) can afford certain therapeutic advantages resulting from greater metabolic stability, for example, increased in vivo half-life, or reduced dosage requirements and, hence may be preferred in some instances.

Isotopically-labeled compounds of the present invention can generally be prepared by standard methods and techniques known to those skilled in the art or by procedures similar to those described in the accompanying Examples substituting appropriate isotopically-labeled reagents in place of the corresponding non-labeled reagent.

The invention also includes any or all metabolites of any of the compounds described. The metabolites may include any chemical species generated by a biotransformation of any of the compounds described, such as intermediates and products of metabolism of the compound, such as would be generated in vivo following administration to a human.

Articles of manufacture comprising a compound described herein, or a salt or solvate thereof, in a suitable container are provided. The container may be a vial, jar, ampoule, preloaded syringe, i.v. bag, and the like.

Preferably, the compounds detailed herein are orally bioavailable. However, the compounds may also be formulated for parenteral (e.g., intravenous) administration.

One or several compounds described herein can be used in the preparation of a medicament by combining the compound or compounds as an active ingredient with a pharmacologically acceptable carrier, which are known in the art. Depending on the therapeutic form of the medication, the carrier may be in various forms. In one variation, the manufacture of a medicament is for use in any of the methods disclosed herein, e.g., for the treatment of cancer.

### General Synthetic Methods

The compounds of the invention may be prepared by a number of processes as generally described below and more specifically in the Examples hereinafter (such as the schemes provided in the Examples below). In the following process descriptions, the symbols when used in the formulae depicted are to be understood to represent those groups described above in relation to the formulae herein.

Where it is desired to obtain a particular enantiomer of a compound, this may be accomplished from a corresponding mixture of enantiomers using any suitable conventional procedure for separating or resolving enantiomers. Thus, for example, diastereomeric derivatives may be produced by reaction of a mixture of enantiomers, e.g., a racemate, and an appropriate chiral compound. The diastereomers may then be separated by any convenient means, for example by crystallization and the desired enantiomer recovered. In another resolution process, a racemate may be separated using chiral High Performance Liquid Chromatography. Alternatively, if desired a particular enantiomer may be obtained by using an appropriate chiral intermediate in one of the processes described.

Chromatography, recrystallization and other conventional separation procedures may also be used with intermediates or final products where it is desired to obtain a particular isomer of a compound or to otherwise purify a product of a reaction.

Solvates and/or polymorphs of a compound provided herein or a pharmaceutically acceptable salt thereof are also contemplated. Solvates contain either stoichiometric or non-stoichiometric amounts of a solvent, and are often formed during the process of crystallization. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and/or solubility. Various factors such as the recrystallization solvent, rate of crystallization, and storage temperature may cause a single crystal form to dominate.

The following abbreviations are used herein: thin layer chromatography (TLC); hour (h); minute (min); ethanol (EtOH); dimethylsulfoxide (DMSO); N,N-dimethylformamide (DMF); trifluoroacetic acid (TFA); tetrahydrofuran (THF); Normal (N); aqueous (aq.); methanol (MeOH); dichloromethane (DCM); ethyl acetate (EtOAc); Retention factor (Rf); room temperature (RT); acetyl (Ac); 4,5-Bis(diphenylphosphino)-9,9-dimethylxanthene (Xantphos); 1-[Bis(dimethylamino)methylene]-1H-1,2,3-triazolo[4,5-b]pyridinium 3-oxid hexafluorophosphate (HATU); N,N-diisopropylamine (DIPEA); N-Bromosuccinimide (NBS); Acetonitrile (ACN); Tetrakis(triphenylphosphine)palladium(0) (Pd[P(C6H5) 3]4, and (tetrakis): meta Chloroperoxybenzoic acid (m-CPBA); 2,2 Bis(diphenylphosphino)-J, 1′-binaphthyl (BINAP).

In some embodiments, compounds of the formula (I) may be synthesized according to Scheme 1 to Scheme 7.

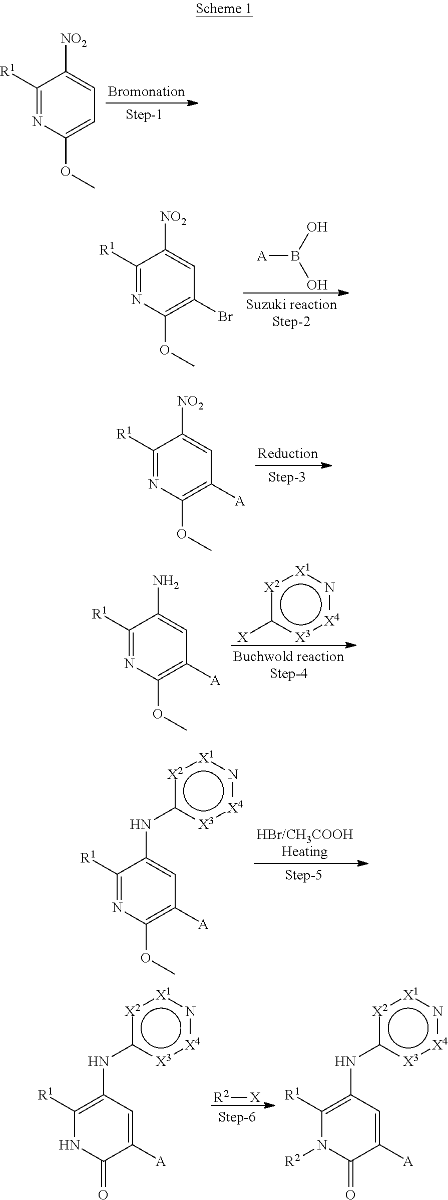

wherein R¹, R², A, X¹, X², X³ and X⁴ are as defined for formula (I) and X is leaving group like halogen.

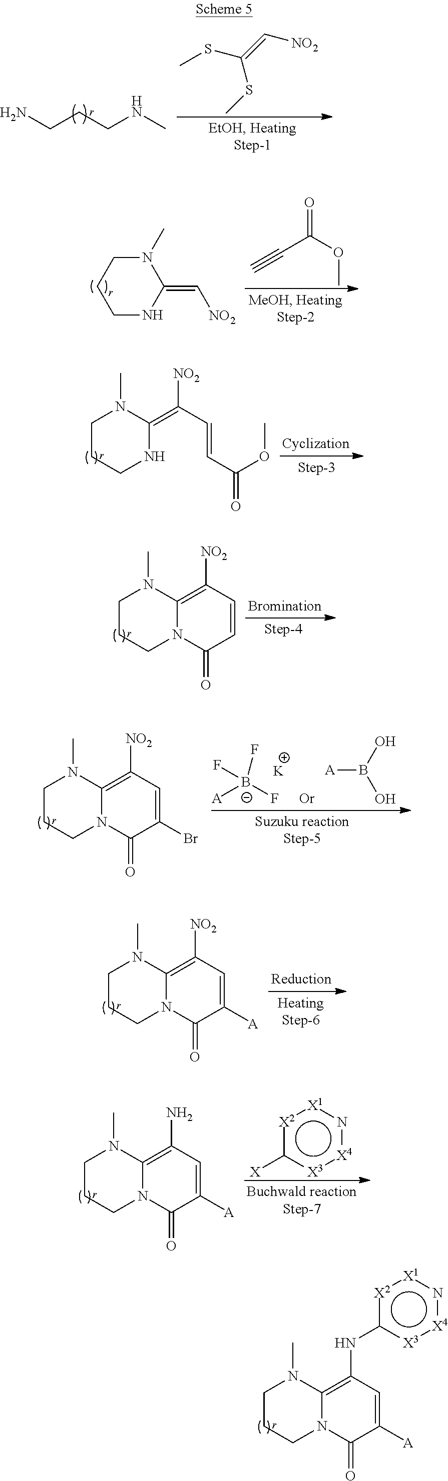

wherein A, X¹, X², X³ and X⁴ are as defined for formula (I), X is leaving group like halogen, and r is 0, 1, or 2.

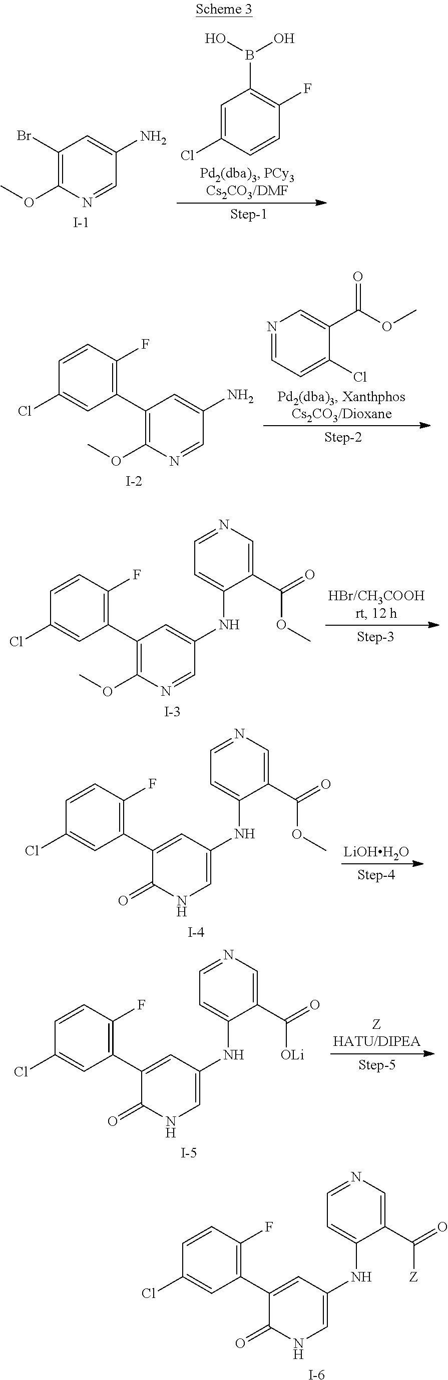

wherein Z is selected from

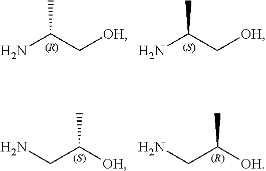

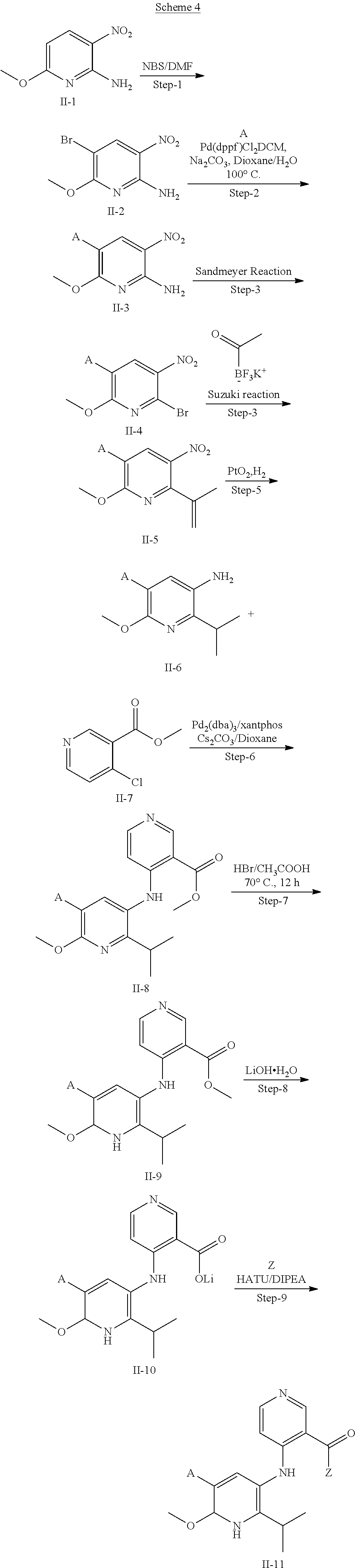

wherein A is selected from

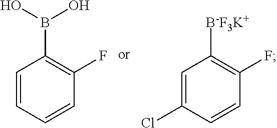

and
Z is selected from

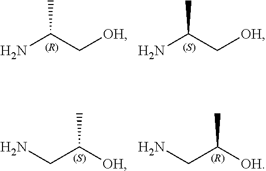

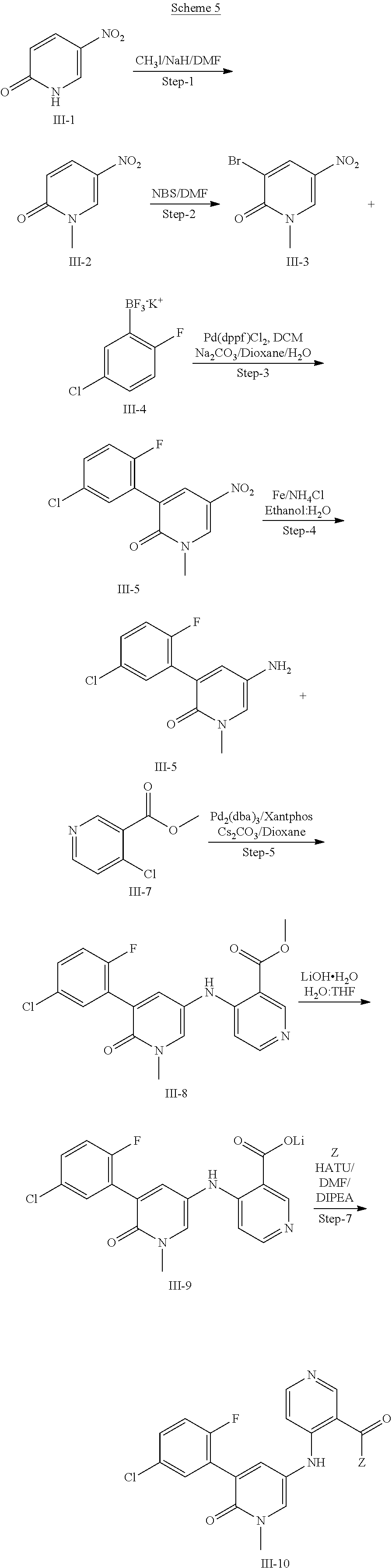

wherein Z is selected from

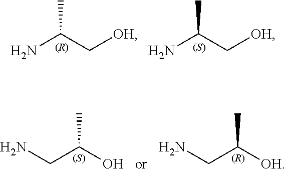

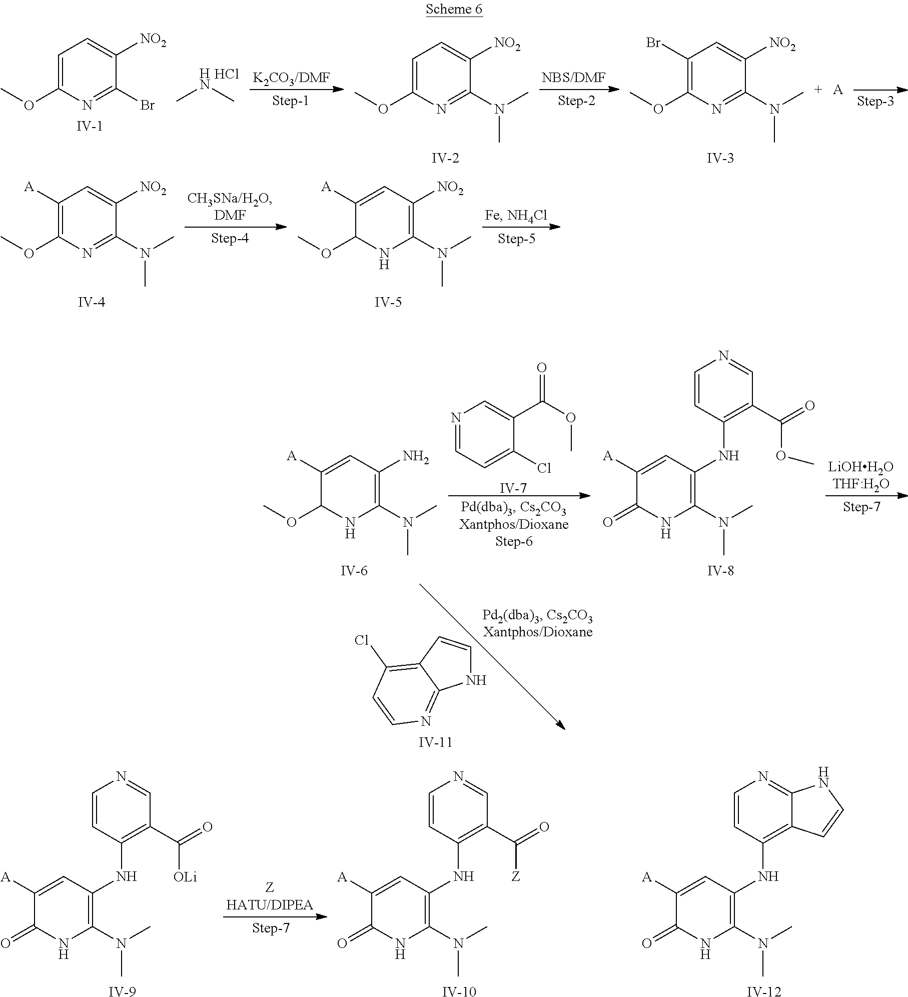

wherein Z is selected from

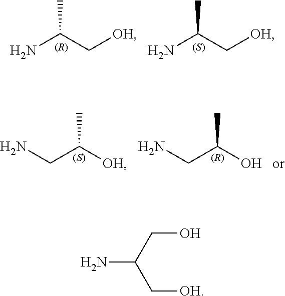

### Note: When A is

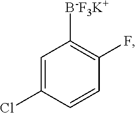

then step-3 was carried out in presence of Pd(dppf)Cl₂DCM, Na₂CO₃, Dioxane/H₂O at 100° C. for overnight.

### When A is

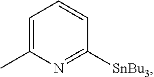

then step-3 was carried out in presence of LiCl, Toluene at 130° C. for 48 h.

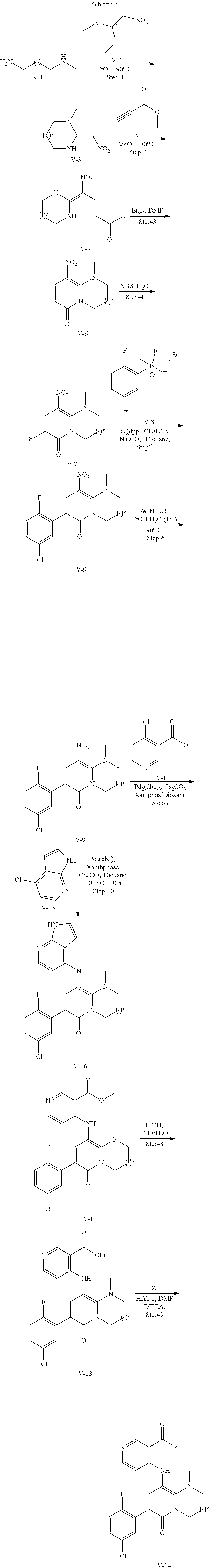

wherein Z is selected from

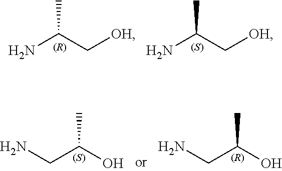

and r is 0, 1, or 2.

It is understood that General Synthetic Schemes 1 to 7 and present synthetic routes involving steps clearly familiar to those skilled in the art, wherein the substituents described in compounds of Formula (I), (II), (II-A) to (II-X), (III), (III-A) to (III-P), (IV), (IV-A) to (IV-P), (V) or (V-A) to (V-L) herein can be varied with a choice of appropriate starting materials and reagents utilized in the steps presented.

### Pharmaceutical Compositions and Formulations

Pharmaceutical compositions of any of the compounds detailed herein are embraced by this disclosure. Thus, the present disclosure includes pharmaceutical compositions comprising a compound as detailed herein or a salt thereof and a pharmaceutically acceptable carrier or excipient. In one aspect, the pharmaceutically acceptable salt is an acid addition salt, such as a salt formed with an inorganic or organic acid. Pharmaceutical compositions may take a form suitable for oral, buccal, parenteral, nasal, topical or rectal administration or a form suitable for administration by inhalation.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form.

In one variation, the compounds herein are synthetic compounds prepared for administration to an individual. In another variation, compositions are provided containing a compound in substantially pure form. In another variation, the present disclosure embraces pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein.

A compound detailed herein or salt thereof may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound or salt thereof may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

One or several compounds described herein or a salt thereof can be used in the preparation of a formulation, such as a pharmaceutical formulation, by combining the compound or compounds, or a salt thereof, as an active ingredient with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington's Pharmaceutical Sciences, Mack Publishing Company, Philadelphia, Pa., 20^{th} ed. (2000), which is incorporated herein by reference.

Compounds as described herein may be administered to individuals in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid poly-ols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

Any of the compounds described herein can be formulated in a tablet in any dosage form described, for example, a compound as described herein or a salt thereof can be incorporated in tablet in an amount ranging from about 1 mg to about 1000 mg.

Compositions comprising a compound provided herein are also described. In one variation, the composition comprises a compound or salt thereof and a pharmaceutically acceptable carrier or excipient. In another variation, a composition of substantially pure compound is provided.

### Methods of Use

Compounds of the present invention can be used to treat disorders and conditions characterized by excessive activity of TGF-β such as fibroproliferation. A “fibroproliferation disorder” affect many tissues and organ systems. Fibroproliferation disorder include fibroproliferation diseases such as interstitial lung diseases, (also known as myeloid metaplasia), heart disease especially cardiac fibrosis and progressive heart failure, hypertensive myelofibrosis, osteoporosis, liver cirrhosis, liver fibrosis resulting from chronic hepatitis B or C infection, kidney disease, systemic sclerosis and vasculopathy where fibrosis is a major cause of morbidity and mortality. Conditions such as atherosclerosis, eye diseases including macular degeneration and retinal and vitreal retinopathy, keloids and hypertrophic scars, nasal polyposis, and restenosis systemic and local scleroderma are also fibroproliferative disorders. Additionally fibrotic disorders include excessive scarring occurring during the healing of wounds, keloid formation, and traumatic lacerations, drugs and radiation induced fibrosis, injuries and burns. Fibrotic tissue remodeling can also influence cancer metastasis and accelerate chronic graft rejection in transplant recipients.

Specifically, TGF-β inhibition can benefit different disease including cardiovascular diseases like congestive heart failure, dilated cardiomyopathy, myocarditis, or vascular stenosis associated with atherosclerosis, angioplasty, atherosclerosis, mechanical trauma or surgical incision, fibrosis and/or sclerosis associated kidney diseases such as glomerulonephritis and cirrhosis of all etiologies, renal interstitial fibrosis, diabetic nephropathy, HIV-associated nephropathy, Nonalcoholic steatohepatitis (NASH), NASH liver failure, fatty liver disease, NASH related metabolic syndrome, NASH related liver cirrhosis, angioplasty hypertension, transplant nephropathy, cirrhosis, chronic ureteral obstruction, complications arising from drug exposure, such as cyclosporin; disorders of the biliary tree, hepatic diseases which arise due to excessive scarring and progressive sclerosis and also includes cirrhosis due to all etiologies, and hepatic dysfunction due to infection from hepatitis virus or parasites; pulmonary fibrosis due to toxic agents such as chemicals, allergens, smoke, or autoimmune disease or its related syndromes, immunogenic tumors including melanoma, loss of gas exchange or ability to move air into and out of the lungs efficiently, adult respiratory distress syndrome, idiopathic pulmonary fibrosis; chronic or persistent collagen vascular disorders including progressive systemic sclerosis, polymyositis, dermatomyositis, scleroderma, Raynaud's syndrome, fascists, eye diseases associated with fibroproliferative states, arthritic conditions such as rheumatoid arthritis; proliferative vitreoretinopathy or fibrosis due to ocular surgery such as retinal reattachment, or any kind of drainage procedures, cataract extraction; hypertrophic or excessive scar formation in the dermis because of wound healing that arise due to trauma or surgical wounds; gastrointestinal tract disorders associated with chronic inflammation such as Crohn's disease, or adhesion formation from surgical wound or trauma, or ulcerative colitis, or polyposis; chronic peritoneum scarring associated with endometriosis, peritoneal dialysis, ovarian disease, or surgical wounds; neurological conditions characterized by enhanced sensitivity to TGF-β or TGF-β production. It also include post-traumatic conditions or hypoxic injury, Parkinson's and Alzheimer's disease; joint disease with scarring that impede mobility or produce pain, including surgical trauma and post-mechanical conditions, rheumatoid arthritis and osteoarthritis; and different type of cancer such as colorectal cancer, lung cancer, skin cancer, breast cancer, prostrate cancer, brain cancer, pancreatic cancer and glioma.

TGF-β modulates the immune and inflammation systems (Wahl et al., Immunol. Today (1989) 10:258-61) by stimulation of leukocyte recruitment, production of cytokines and lymphocyte effector function, inhibition of T-cell subset proliferation, inhibition of B-cell proliferation, formation of antibodies and monocytic respiratory burst. TGF-β stimulates the excess production of extracellular matrix proteins, including collagen and fibronectin. It also inhibits enzymes production that degrades these matrix proteins. Ultimately there is accumulation of fibrous tissue which is the characteristic of fibroproliferative diseases.

In one aspect, the invention provides a method for inhibition of TGF-β receptor kinase, comprise of administering to an individual an effective amount of one or more compounds of the invention. In one aspect of the method, binding of a ligand to the TGF-β receptor is inhibited by compounds of the invention, or a salt thereof (e.g., a pharmaceutically acceptable salt) and/or reduces or eliminates or increases or enhances or mimics an activity of the TGF-β receptor reversibly or irreversibly. In some aspects, a compound of the invention inhibits binding of a ligand to the TGF-β receptor by at least about or by about any one of 10%, 20%, 30%, 40%, 50%, 60%, 70%, 80%, 90%, 95% or 100% as determined by an assay described herein. In some aspects, a compound of the invention reduces an activity of the TGF-β receptor by at least about or about any of 10%, 20%, 30%, 40%, 50%, 60%, 70%), 80%, 90%, 95% or 100% as compared to the corresponding activity in the same subject prior to treatment with the receptor modulator or compared to the corresponding activity in other subjects not receiving the compound. In one aspect, the individual has or is believed to have a disorder in which the TGF-β receptor is implicated. In certain variations, a compound or composition of the invention is used to treat or prevent a TGF-β receptor related disorder, such as cancer (e.g., neuroblastoma, pancreatic cancer and colon cancer). In one aspect, the method comprises administering to the individual a compound provided herein, or a pharmaceutically acceptable salt thereof, including but not limited to a compound of the invention such as a compound according to any one or more of formula (I), (II), (II-A) to (II-X), (III), (III-A) to (III-P), (IV), (IV-A) to (IV-P), (V) or (V-A) to (V-L) or a compound of Tables 1 or 2, or an isomer thereof, or a salt (such as a pharmaceutically acceptable salt) of any of the foregoing. In one aspect, the individual is a human in need of cancer treatment.

Examples of cell-based assays and in-vitro assays are provided below. Bleomycin-induced pulmonary fibrosis models {e.g., Peng et al., PLoS ONE 8(4), e59348, 2013; Izbicki et al, Int. J. Exp. Path. 83, 111-19, 2002); colorectal cancer models (e.g., Zigmond et al., PLoS ONE 6(12), e28858, 2011); and bone metastasis models (e.g., Mohammad et al., Cancer Res. 71, 175-84, 2011; Buijs et al., BoneKEy Reports 1, Article number: 96, 2012) are appropriate and accepted animal model that are well known in the art.

In some embodiments, examples such as sarcomas and carcinomas are cancer that may be treated as solid tumors. In some embodiments, examples such as leukemia are the cancer that may be treated as liquid tumors. Present invention may treat different types of cancers that include, but are not limited to, adrenocortical cancer, bladder cancer, brain tumors, breast cancer, prostrate cancer, colorectal cancer, colon cancer, endometrial cancer, gallbladder cancer, gastric cancer, head and neck cancer, hematopoietic cancer, kidney cancer, leukemia, oral cancer, uterine carcinoma, Hodgkin lympoma, liver cancer, lung cancer, pancreatic cancer, prostate cancer, ovarian cancer, sarcoma, skin cancer and thyroid cancer. In some embodiments, the breast cancer is classified as carcinoma of breast (ER negative or ER positive), mammary adenocarcinoma, primary breast ductal carcinoma, mammary ductal carcinoma (ER positive, ER negative or HER2 positive), triple negative breast cancer (TNBC), HER2 positive breast cancer or luminal breast cancer. In some embodiments, the breast cancer is unclassified. In some embodiments, a basal-like TNBC, an immunomodulatory TNBC, mesenchymal TNBC (mesenchymal or mesenchymal stem-like) or a luminal androgen receptor TNBC are triple negative breast. In some embodiments, prostate adenocarcinoma is prostate cancer. In some embodiments, the ovary adenocarcinoma is ovarian cancer. In some embodiments, lung carcinoma, adenocarcinoma, non-small lung carcinoma, mucoepidermoid, anaplastic large cell are lung cancer. In some embodiments, the lung cancer is unclassified. In some embodiments, the colon adenocarcinomas, colon carcinoma, metastatic colorectal cancer, colon adenocarcinoma from a metastatic site lymph node are colon cancer. In some embodiments astrocytoma, glioblastoma, meduloblastoma, neuroblastoma or meningioma is brain tumor. In some embodiments, stomach cancer is gastric cancer. In some embodiments, cholangiocarcinoma or hepatoblastoma, hepatocellular carcinoma are liver cancers. In some embodiments, liver cancer is derived from hepatitis B virus. In some embodiments, liver cancer is virus negative. In some embodiments, medullary thyroid cancer or follicular thyroid cancer, papillary thyroid carcinomas are classified as thyroid cancer. In some embodiments, uterine papillary serous carcinoma or uterine clear cell carcinoma, high grade endometroid cancer are endometrial cancer. In some embodiments, gallbladder adenocarcinoma or squamous cell gallbladder carcinoma are gallbladder cancer. In some embodiments, renal cell carcinoma or urothelial cell carcinoma are classified as kidney cancer. In some embodiments, adrenal cortical carcinoma adrenocortical is cancer. In some embodiments, fibrosarcoma or Ewing's sarcoma, osteosarcoma, rhabdomiosarcoma and synovial sarcoma are classified as sarcoma. In some embodiments, basal cell carcinoma, melanoma or squamous carcinoma are classified as skin cancer. In some embodiments, cancer of the trachea, laryngeal cancer, nasopharyngeal cancer and oropharyngeal cancer are classified as head and neck cancer. In some embodiments, acute lymphoblastic leukemia, acute promyelocytic leukemia, chronic myelogenous leukemia, chronic lymphocytic leukemia, mantle cell lymphoma or multiple myeloma are classified as leukemia.

In the present invention, compound of the present invention are used to treat nonalcoholic steatohepatitis (NASH), NASH liver failure, fatty liver disease, NASH related metabolic syndrome and/or NASH related liver cirrhosis.

In the present invention, tumor is treated with effective amount of one or more compound of invention, or a salt thereof. In one aspect of the method, tumor is treated by administrating a compound or salt thereof to an individual in need of tumor treatment. Exemplary tumors are derived from carcinomas of the breast, prostate, ovary, lung, or colon. In one aspect, the treatment results in a reduction of the tumor size. In another aspect, the treatment slows or prevents tumor growth and/or metastasis.

The present invention also provides a method to treat hematopoietic malignancy by administrating an effective amount of one or more compounds of the invention to an individual in need thereof. In some embodiments, acute promyelocytic leukemia is classified as hematopoietic malignancy.

Individual (e.g., human) who are diagnosed with or suspect cancer may be treated with any of the methods described in the present invention. In some embodiments, individuals are described as humans that exhibits one or more symptoms associated with cancer. In some embodiments, individuals exhibit advanced stage of the disease such as cancer or to a lesser extent with low tumor burden. In some embodiments, the individual is at an early stage of a cancer. In some of the embodiments, individuals may be humans that are genetically predisposed to develop cancer (has or has not been diagnosed with cancer). In some of the embodiments, individual may be humans that are otherwise predisposed (e.g., has one or more so-called risk factors) to develop cancer (has or has not been diagnosed with cancer). In some embodiments, age, diet, history of previous disease, environmental exposure, genetic (e.g., hereditary) considerations, presence of precursor disease, race, sex etc. are various risk factors. In some embodiments, the individuals who are at risk of cancer include relatives who experienced this disease or those that are analyzed by genetic or biochemical markers. In some embodiments, the individual does not have type I diabetes or type II diabetes (with sustained hyperglycemia or hyperglycemia with prolonged duration for several years).

In some embodiments, compounds of the invention is administered to a patient alone or in combination with one or more cancer immunotherapies such as antibody therapies, cell-based therapies (“cancer vaccines”), cytokine therapies, and other immunosuppressive mediators such as indoleamine 2,3-dioxygenase (IDO). Unless otherwise indicated, “in combination” as used herein includes substantially simultaneous administration of the compound of the invention and one or more cancer immunotherapies (either in the same composition or in separate compositions) as well as sequential administration.

Cell-based therapies include, but are not limited to, dendritic cells, natural killer cells, lymphokine-activated killer cells, cytotoxic T cells, regulatory T cells, and in some embodiments, a compound of the invention is used in combination with sipuleucel-T (e.g., PROVENGE®), to treat prostate cancer. In some embodiments, adjuvants, such as GM-CSF, are used to attract and/or activate dendritic cells.

Antibody therapies include, but are not limited to, antibodies to cell surface receptors, such as epidermal growth factor receptor and HER2, as well as antibodies which block immune checkpoints (e.g., antibodies which bind to molecules such as PD-1, PD-L1, and CTLA-4). The term “antibody” as used herein includes monoclonal antibodies, humanized or chimeric antibodies, bispecific antibodies (e.g., BiTE), single chain antibodies, and binding fragments such as Fab, Fab′ F(ab′)₂, Fabc, and Fv. Antibodies may be used alone or may be conjugated, for example, to a moiety which is either toxic to cells (antibody drug conjugate, or ADC) or is radioactive. Examples of antibody therapies include Pidilizumab, Alemtuzumab, Bevacizumab, Brentuximab vedotin, Cetuximab, Gemtuzumab ozogamicin, Ibritumomab tiuxetan, Ipilimumab, Ofatumumab, Panitumumab, Rituximab, Tositumomab, and Trastuzumab.

Cytokine therapies include, but are not limited to, GM-CSF, interleukins (e.g., IL-2, IL-7, IL-10, IL-12, IL-15, IL-18, IL-21), and interferons (e.g., interferon α).

### Dosing and Method of Administration

The dose of a compound administered to an individual (such as a human) may vary with the particular compound or salt thereof, the method of administration, and the particular disease, such as type and stage of cancer, being treated. In some embodiments, the amount of the compound or salt thereof is a therapeutically effective amount.

The effective amount of the compound may in one aspect be a dose of between about 0.01 and about 100 mg/kg. Effective amounts or doses of the compounds of the invention may be ascertained by routine methods, such as modeling, dose escalation, or clinical trials, taking into account routine factors, e.g., the mode or route of administration or drug delivery, the pharmacokinetics of the agent, the severity and course of the disease to be treated, the subject's health status, condition, and weight. An exemplary dose is in the range of about from about 0.7 mg to 7 g daily, or about 7 mg to 350 mg daily, or about 350 mg to 1.75 g daily, or about 1.75 to 7 g daily.

Any of the methods provided herein may in one aspect comprise administering to an individual a pharmaceutical composition that contains an effective amount of a compound provided herein or a salt thereof and a pharmaceutically acceptable excipient.

A compound or composition of the invention may be administered to an individual in accordance with an effective dosing regimen for a desired period of time or duration, such as at least about one month, at least about 2 months, at least about 3 months, at least about 6 months, or at least about 12 months or longer, which in some variations may be for the duration of the individual's life. In one variation, the compound is administered on a daily or intermittent schedule. The compound can be administered to an individual continuously (for example, at least once daily) over a period of time. The dosing frequency can also be less than once daily, e.g., about a once weekly dosing. The dosing frequency can be more than once daily, e.g., twice or three times daily. The dosing frequency can also be intermittent, including a ‘drug holiday’ (e.g., once daily dosing for 7 days followed by no doses for 7 days, repeated for any 14 day time period, such as about 2 months, about 4 months, about 6 months or more). Any of the dosing frequencies can employ any of the compounds described herein together with any of the dosages described herein.

The compounds provided herein or a salt thereof may be administered to an individual via various routes, including, e.g., intravenous, intramuscular, subcutaneous, oral and transdermal. A compound provided herein can be administered frequently at low doses, known as ‘metronomic therapy,’ or as part of a maintenance therapy using compound alone or in combination with one or more additional drugs. Metronomic therapy or maintenance therapy can comprise administration of a compound provided herein in cycles. Metronomic therapy or maintenance therapy can comprise intra-tumoral administration of a compound provided herein.

In one aspect, the invention provides a method of treating cancer in an individual by parenterally administering to the individual (e.g., a human) an effective amount of a compound or salt thereof. In some embodiments, the route of administration is intravenous, intra-arterial, intramuscular, or subcutaneous. In some embodiments, the route of administration is oral. In still other embodiments, the route of administration is transdermal.

The invention also provides compositions (including pharmaceutical compositions) as described herein for the use in treating, preventing, and/or delaying the onset and/or development of cancer and other methods described herein. In certain embodiments, the composition comprises a pharmaceutical formulation which is present in a unit dosage form.

Also provided articles of manufacture comprising a compound of the disclosure or a salt thereof, composition, and unit dosages described herein in suitable packaging for use in the methods described herein. Suitable packaging is known in the art and includes, for example, vials, vessels, ampules, bottles, jars, flexible packaging and the like. An article of manufacture may further be sterilized and/or sealed.

### Kits

The present disclosure further provides kits for carrying out the methods of the invention, which comprises one or more compounds described herein or a composition comprising a compound described herein. The kits may employ any of the compounds disclosed herein. In one variation, the kit employs a compound described herein or a pharmaceutically acceptable salt thereof. The kits may be used for any one or more of the uses described herein, and, accordingly, may contain instructions for the treatment of cancer.

Kits generally comprise suitable packaging. The kits may comprise one or more containers comprising any compound described herein. Each component (if there is more than one component) can be packaged in separate containers or some components can be combined in one container where cross-reactivity and shelf life permit.

The kits may be in unit dosage forms, bulk packages (e.g., multi-dose packages) or sub-unit doses. For example, kits may be provided that contain sufficient dosages of a compound as disclosed herein and/or a second pharmaceutically active compound useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present invention. The instructions included with the kit generally include information as to the components and their administration to an individual.

The invention can be further understood by reference to the following examples, which are provided by way of illustration and are not meant to be limiting.

### EXAMPLES

### Example-1: Synthesis of 4-((5-(5-chloro-2-fluorophenyl)-6-oxo-1,6-dihydropyridin-3-yl)amino)-N-(2-hydroxypropyl)nicotinamide (Compound 1.1)

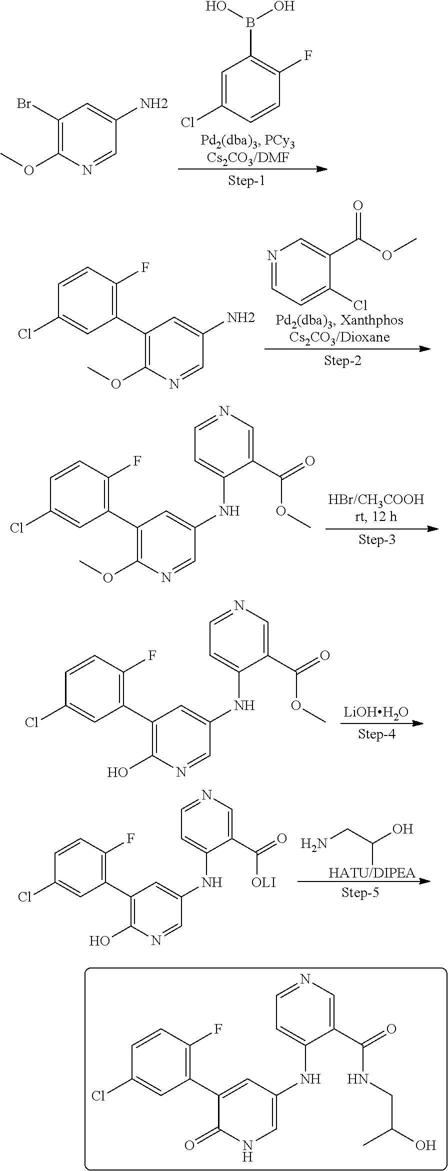

### Step-1: Synthesis of 5-(5-chloro-2-fluorophenyl)-6-methoxypyridin-3-amine

To a stirred solution of 5-bromo-6-methoxypyridin-3-amine (1.0 g, 4.925 mmol, 1.0 eq) and (5-chloro-2-fluorophenyl)boronic acid (1.28 g, 7.387 mmol, 1.5 eq) in DMF (20 mL) was added Cs₂CO₃ (2.73 g, 8.372 mmol, 1.7 eq) at RT. The resulting mixture was purged with nitrogen for 10 min followed by addition of Pd₂(dba)₃ (0.144 g, 0.157 mmol, 0.032 eq) and PCy₃ (0.102 g, 0.364 mmol, 0.074 eq) and again purged with nitrogen for 10 min. The reaction mixture was heated at 100° C. for overnight. The progress of reaction was monitored by LCMS. The reaction mixture was diluted with ice cold water (50 mL), extracted with EtOAc (2×100 mL). The combined organic layers were washed with water (50 mL), with brine (50 mL), dried over Na₂SO₄, concentrated and purified by combi flash chromatography [silica gel 100-200 mesh: elution 0-20% EtOAc in Hexane] to afford the desired compound 5-(5-chloro-2-fluorophenyl)-6-methoxypyridin-3-amine (800 mg, 64.30%) as brown solid. LCMS: (M+1)⁺: 252.9

### Step-2: Synthesis of methyl 4-((5-(5-chloro-2-fluorophenyl)-6-methoxypyridin-3-yl)amino)nicotinate

To a stirred solution of 5-(5-chloro-2-fluorophenyl)-6-methoxypyridin-3-amine (0.8 g, 3.166 mmol, 1.0 eq) and methyl 4-chloronicotinate (0.543 g, 3.166 mmol, 1.0 eq) in dioxane (30 mL) was added Cs₂CO₃ (4.13 g, 12.664 mmol, 4.0 eq) at RT. The resulting mixture was purged with nitrogen for 10 min followed by addition of Pd₂(dba)₃ (0.348 g, 0.379 mmol, 0.12 eq) and xatphos (0.275 g, 0.475 mmol, 0.15 eq) and again purged with nitrogen for 10 min. The reaction mixture was heated at 100° C. for overnight. The progress of reaction was monitored by LCMS. The reaction mixture was diluted with water (50 mL), extracted with EtOAc (2×100 mL). The combined organic layers were washed with water (50 mL), with brine (50 mL), dried over Na₂SO₄, concentrated and purified by combi flash chromatography [silica gel 100-200 mesh: elution 0-30% EtOAc in Hexane] to afford the desired compound 4-((5-(5-chloro-2-fluorophenyl)-6-methoxypyridin-3-yl)amino)nicotinate (600 mg, 48.78%) as brown solid. LCMS: (M+1)⁺: 388.2.

### Step-3: Synthesis of methyl 4-((5-(5-chloro-2-fluorophenyl)-6-hydroxypyridin-3-yl)amino)nicotinate

To a stirred solution of 4-((5-(5-chloro-2-fluorophenyl)-6-methoxypyridin-3-yl)amino)nicotinate (0.6 g, 1.547 mmol, 1.0 eq) in acetic acid (0.078 M) was added 48% aqueous HBr solution (0.782 mL, 4.641 mmol, 3.0 eq) at RT. The reaction mixture was heated at 80° C. for overnight. The progress of reaction was monitored by LCMS. The reaction mixture was basified with saturated aqueous solution of NaHCO₃ (50 mL), extracted with EtOAc (2×100 mL). The combined organic layers were washed with water (50 mL), with brine (50 mL), dried over Na₂SO₄, concentrated and purified by combi flash chromatography [silica gel 100-200 mesh: elution 0-5% MeOH in DCM] to afford the desired compound methyl 4-((5-(5-chloro-2-fluorophenyl)-6-hydroxypyridin-3-yl)amino)nicotinate (400 mg, 69.20%) as brown solid. LCMS: (M+1)⁺: 374.3.

### Step-4: Synthesis of lithium 4-((5-(5-chloro-2-fluorophenyl)-6-hydroxypyridin-3-yl)amino)nicotinate

To a stirred solution of 4-((5-(5-chloro-2-fluorophenyl)-6-hydroxypyridin-3-yl)amino)nicotinate (0.480 g, 1.284 mmol, 1.0 eq) in THF (4.0 mL) was added a solution of LiOH.H₂O (0.108 g, 2.568 mmol, 2.0 eq) in water (1.0 mL) at RT. The reaction mixture was stirred at RT for overnight. The progress of reaction was monitored by LCMS. The reaction mixture was concentrated to afford the desired lithium 4-((5-(5-chloro-2-fluorophenyl)-6-hydroxypyridin-3-yl)amino)nicotinate (450 mg, 95.94%) as brown solid. LCMS: (M+1)⁺: 360.2.

### Step-5: Synthesis of 4-((5-(5-chloro-2-fluorophenyl)-6-oxo-1,6-dihydropyridin-3-yl)amino)-N-(2-hydroxypropyl)nicotinamide

To a stirred solution of lithium 4-((5-(5-chloro-2-fluorophenyl)-6-hydroxypyridin-3-yl)amino)nicotinate (0.20 g, 0.546 mmol, 1.0 eq) in DMF (5 mL) was added HATU (0.311 g, 0.819 mmol, 1.5 eq) at RT. The resulting mixture was stirred at RT for 5 minutes, followed by addition of 1-aminopropan-2-ol (0.082 g, 1.093 mmol, 2.0 eq) and DIPEA (0.285 mL, 1.638 mmol, 3.0 eq) and stirred at RT for 1 h. Progress of reaction was monitored by LCMS. Reaction mixture was diluted with water (50 mL) and extracted with ethyl acetate (2×50 mL). The combined organic extracts were washed with water (50 mL), with brine (50 mL) dried over Na₂SO₄ and concentrated under reduced and purified by combi flash chromatography [silic gel 100-200 mesh; elution 0-10% MeOH in DCM] to afford the desired compound 4-((5-(5-chloro-2-fluorophenyl)-6-oxo-1,6-dihydropyridin-3-yl)amino)-N-(2-hydroxypropyl)nicotinamide (60 mg, 26.31%) as off white solid. LCMS: (M+1)⁺: 417.3. UPLC: At 254 nm=97.10%, At 220 nm=98.33%

¹H NMR (400 MHz, DMSO-d₆): δ 12.01 (brs, 1H), 9.58 (s, 1H), 8.67 (s, 1H), 8.60 (brs, 1H), 8.15 (d, J=6.14 Hz, 1H), 7.59-7.70 (m, 1H), 7.56 (brs, 1H), 7.51 (brs, 1H), 7.44 (brs, 1H), 7.30 (t, J=9.43 Hz, 1H), 6.64 (d, J=6.14 Hz, 1H), 4.77 (d, J=4.82 Hz, 1H), 3.72-3.88 (m, 1H), 3.20 (d, J=4.39 Hz, 2H), 1.08 (d, J=6.14 Hz, 3H).

### Example-2: Synthesis of (S)-4-((5-(5-chloro-2-fluorophenyl)-6-oxo-1,6-dihydropyridin-3-yl)amino)-N-(1-hydroxypropan-2-yl)nicotinamide: (Compound 1.2)

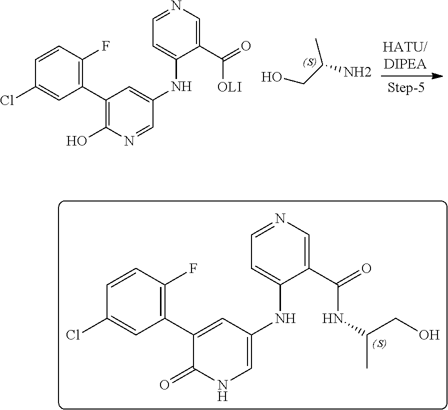

Steps 1 to 4 are the same as in Example 1.

### Step-5: Synthesis of (S)-4-((5-(5-chloro-2-fluorophenyl)-6-oxo-1,6-dihydropyridin-3-yl)amino)-N-(1-hydroxypropan-2-yl)nicotinamide

To a stirred solution of lithium 4-((5-(5-chloro-2-fluorophenyl)-6-hydroxypyridin-3-yl)amino)nicotinate (0.250 g, 0.683 mmol, 1.0 eq) in DMF (5 mL) was added HATU (0.390 g, 1.025 mmol, 1.5 eq) at RT. The resulting mixture was stirred at RT for 5 minutes, followed by addition of (S)-2-aminopropan-1-ol (0.102 g, 1.367 mmol, 2.0 eq) and DIPEA (0.36 mL, 2.049 mmol, 3.0 eq) and stirred at RT for 1 h. Reaction mixture was diluted with water (50 mL) and extracted with ethyl acetate (2×50 mL). The combined organic extracts were washed with water (50 mL), with brine (50 mL) dried over Na₂SO₄ and concentrated under reduced and purified by combi flash chromatography [silic gel 100-200 mesh; elution 0-10% MeOH in DCM] to afford the desired compound (S)-4-((5-(5-chloro-2-fluorophenyl)-6-oxo-1,6-dihydropyridin-3-yl)amino)-N-(1-hydroxypropan-2-yl)nicotinamide (60 mg, 26.31%) as off white solid. LCMS: (M+1)⁺:417.2. UPLC: At 254 nm=95.17%; At 220 nm=95.68%.

¹H NMR (400 MHz, DMSO-d₆): δ 12.03 (brs, 1H), 9.59 (s, 1H), 8.66 (s, 1H), 8.31 (d, J=7.89 Hz, 1H), 8.15 (d, J=5.70 Hz, 1H), 7.63 (dd, J=2.63, 6.14 Hz, 1H), 7.56 (s, 1H), 7.52 (brs, 1H), 7.45 (dd, J=3.73, 8.11 Hz, 1H), 7.30 (t, J=9.21 Hz, 1H), 6.64 (d, J=6.14 Hz, 1H), 4.76 (t, J=5.92 Hz, 1H), 3.89-4.11 (m, 1H), 3.47 (td, J=5.76, 10.85 Hz, 1H), 3.35-3.40 (m, 1H), 1.13 (d, J=6.58 Hz, 3H).

### Example-3: Synthesis of (S)-4-((5-(2-fluorophenyl)-2-isopropyl-6-oxo-1,6-dihydropyridin-3-yl)amino)-N-(2-hydroxypropyl)nicotinamide: (Compound 1.3)

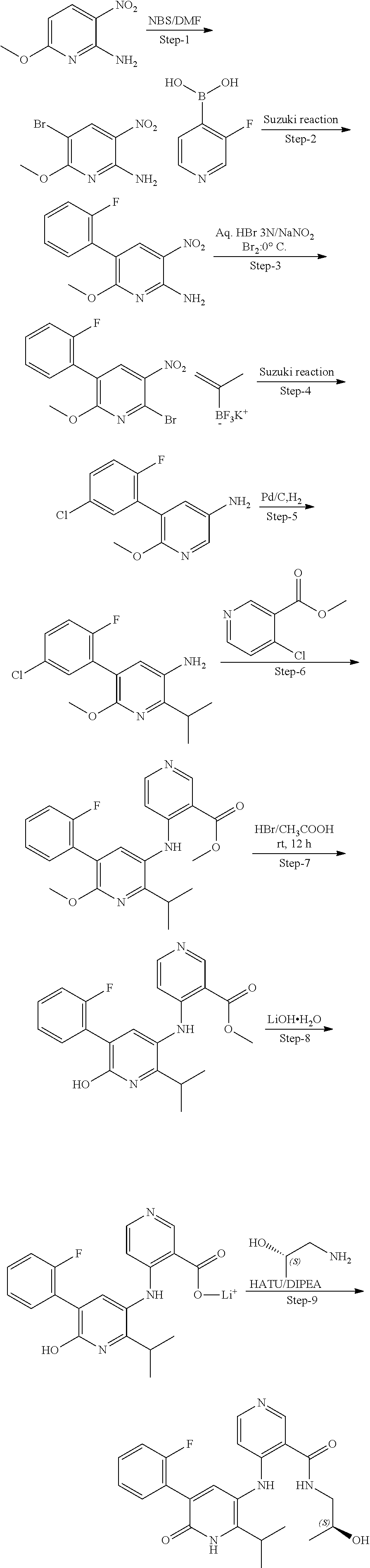

### Step-1: Synthesis of 5-bromo-6-methoxy-3-nitropyridin-2-amine

To a stirred solution of 6-methoxy-3-nitropyridin-2-amine (5.0 g, 29.57 mmol, 1.0 eq) in DMF (50 mL) was added NBS (5.78 g, 32.517 mmol, 3.0 eq) at RT. The reaction mixture was stirred at RT for 1 h. The progress of reaction was monitored by LCMS. The reaction mixture was poured in ice cold water (50 mL), filtered and the residue was washed with water (50 mL), dried under vacuum to afford the desired compound 5-bromo-6-methoxy-3-nitropyridin-2-amine (7.2 g, 98.22%) as brown solid. LCMS: (M+1)⁺: 248.0.

### Step-2: Synthesis of 5-(2-fluorophenyl)-6-methoxy-3-nitropyridin-2-amine

To a stirred solution of 5-bromo-6-methoxy-3-nitropyridin-2-amine (3.0 g, 12.095 mmol, 1.0 eq) and (2-fluorophenyl)boronic acid (2.19 g, 15.723 mmol, 1.3 eq) in dioxane (30 mL) was added a 2M solution of Na₂CO₃ (2.56 g, 24.19 mmol, 2.0 eq) in water (12 mL) at RT. The resulting mixture was purged with nitrogen for 10 min followed by addition of Pd(dppf)Cl₂.DCM (0.494 g, 0.604 mmol, 0.05 eq), again purged with nitrogen for 10 min. The reaction mixture was heated at 100° C. for overnight. The progress of reaction was monitored by LCMS. The reaction mixture was diluted with ice cold water (50 mL), extracted with EtOAc (2×100 mL). The combined organic layers were washed with water (50 mL), with brine (50 mL), dried over Na₂SO₄, concentrated and purified by combi flash chromatography [silica gel 100-200 mesh: elution 0-10% EtOAc in Hexane] to afford the desired compound 5-(2-fluorophenyl)-6-methoxy-3-nitropyridin-2-amine (2.6 g, 81.42%) as yellow solid. LCMS: (M+1)⁺: 264.2.

### Step-3: Synthesis of 2-bromo-5-(2-fluorophenyl)-6-methoxy-3-nitropyridine

To a stirred solution of 5-(2-fluorophenyl)-6-methoxy-3-nitropyridin-2-amine (2.6 g, 9.877 mmol, 1.0 eq) in 3N aqueous HBr (60 mL) was added NaNO₂ (6.8 g, 98.772 mmol, 10.0 eq) at 0° C. The resulting mixture was stirred for 2 h at the same temperature followed by addition of bromine (0.506 mL, 9.877 mmol, 1.0 eq). The reaction mixture was stirred at 0° C. for 2 h. The progress of reaction was monitored by LCMS. The reaction mixture was basified with saturated solution of NaHCO₃ (50 mL), extracted with EtOAc (2×100 mL). The combined organic layers were washed with water (50 mL), with brine (50 mL), dried over Na₂SO₄, concentrated and purified by combi flash chromatography [silica gel 100-200 mesh: elution 0-10% EtOAc in Hexane] to afford the desired compound 2-bromo-5-(2-fluorophenyl)-6-methoxy-3-nitropyridine (1.78 g, 55.10%) as yellow solid. LCMS: (M+1)⁺: 327.2.

### Step-4: Synthesis of 3-(5-chloro-2-fluorophenyl)-2-methoxy-5-nitro-6-(prop-1-en-2-yl)pyridine

To a stirred solution of 2-bromo-5-(2-fluorophenyl)-6-methoxy-3-nitropyridine (1.30 g, 3.974 mmol, 1.0 eq) and potassium trifluoro(prop-1-en-2-yl)borate (0.705 g, 4.769 mmol, 1.2 eq) in methanol (150 mL) was added triethylamine (0.6 mL, 3.974 mmol, 1.0 eq) at RT. The resulting mixture was purged with nitrogen for 10 min followed by addition of Pd(dppf)Cl₂.DCM (0.162 g, 0.1987 mmol, 0.05 eq) and again purged with nitrogen for 10 min. The reaction mixture was heated at 100° C. for overnight. The progress of reaction was monitored by LCMS. The reaction mixture was concentrated, diluted with water (50 mL), extracted with EtOAc (2×100 mL). The combined organic layers were washed with water (50 mL), with brine (50 mL), dried over Na₂SO₄, concentrated and purified by combi flash chromatography [silica gel 100-200 mesh: elution 0-20% EtOAc in Hexane] to afford the desired compound 3-(5-chloro-2-fluorophenyl)-2-methoxy-5-nitro-6-(prop-1-en-2-yl)pyridine (1.0 g, 86.95%) as yellow solid. LCMS: (M+1)⁺: 289.2.

### Step-5: Synthesis of 5-(5-chloro-2-fluorophenyl)-2-isopropyl-6-methoxypyridin-3-amine

To a stirred solution of 3-(5-chloro-2-fluorophenyl)-2-methoxy-5-nitro-6-(prop-1-en-2-yl)pyridine (1.0 g, 3.468 mmol, 1.0 eq) in methanol (30 mL) was added 10% Pd/C (120 mg) at RT. The reaction mixture was purged with hydrogen for 1 h. The progress of reaction was monitored by LCMS. The reaction mixture was filtered and the residue was washed with methanol (50 mL), filtrate was concentrated to afford the desired compound 5-(5-chloro-2-fluorophenyl)-2-isopropyl-6-methoxypyridin-3-amine (832 g, 92.23%) as brown solid. LCMS: (M+1)⁺: 261.3.

### Step-6: Synthesis of methyl 4-((5-(2-fluorophenyl)-2-isopropyl-6-methoxypyridin-3-yl)amino)nicotinate

To a stirred solution of compound 5-(5-chloro-2-fluorophenyl)-2-isopropyl-6-methoxypyridin-3-amine (0.832 g, 3.196 mmol, 1.0 eq) and methyl 4-chloronicotinate (0.548 g, 3.196 mmol, 1.0 eq) in dioxane (30 mL) was added Cs₂CO₃ (4.166 g, 12.784 mmol, 4.0 eq) at RT. The resulting mixture was purged with nitrogen for 10 min followed by addition of Pd₂(dba)₃ (0.351 g, 0.383 mmol, 0.12 eq) and xatphos (0.277 g, 0.479 mmol, 0.15 eq) and again purged with nitrogen for 10 min. The reaction mixture was heated at 100° C. for overnight. The progress of reaction was monitored by LCMS. The reaction mixture was diluted with water (50 mL), extracted with EtOAc (2×100 mL). The combined organic layers were washed with water (50 mL), with brine (50 mL), dried over Na₂SO₄, concentrated and purified by combi flash chromatography [silica gel 100-200 mesh: elution 0-30% EtOAc in Hexane] to afford the desired compound methyl 4-((5-(2-fluorophenyl)-2-isopropyl-6-methoxypyridin-3-yl)amino)nicotinate (420 mg, 33.33%) as brown solid. LCMS: (M+1)⁺: 396.4.

### Step-7: Synthesis of methyl 4-((5-(2-fluorophenyl)-6-hydroxy-2-isopropylpyridin-3-yl)amino)nicotinate

To a stirred solution of methyl 4-((5-(2-fluorophenyl)-2-isopropyl-6-methoxypyridin-3-yl)amino)nicotinate (0.42 g, 1.062 mmol, 1.0 eq) in acetic acid (0.078 M) was added 48% aqueous HBr solution (0.53 mL, 3.188 mmol, 3.0 eq) at RT. The reaction mixture was stirred at RT for overnight. The progress of reaction was monitored by LCMS. The reaction mixture was basified with saturated aqueous solution of NaHCO₃ (50 mL), extracted with EtOAc (2×100 mL). The combined organic layers were washed with water (50 mL), with brine (50 mL), dried over Na₂SO₄, concentrated and purified by combi flash chromatography [silica gel 100-200 mesh: elution 0-5% MeOH in DCM] to afford the desired compound 4-((5-(2-fluorophenyl)-6-hydroxy-2-isopropylpyridin-3-yl)amino)nicotinate (380 mg, 95.00%) as brown solid. LCMS: (M+1)⁺: 382.4.

### Step-8: Synthesis of lithium 4-((5-(2-fluorophenyl)-6-hydroxy-2-isopropylpyridin-3-yl)amino)nicotinate

To a stirred solution of methyl 4-((5-(2-fluorophenyl)-6-hydroxy-2-isopropylpyridin-3-yl)amino)nicotinate (0.380 g, 0.9963 mmol, 1.0 eq) in THF (8.0 mL) was added a solution of LiOH.H₂O (0.084 g, 1.992 mmol, 2.0 eq) in water (2.0 mL) at RT. The reaction mixture was stirred at for overnight. The progress of reaction was monitored by LCMS. The reaction mixture was concentrated to afford the desired lithium 4-((5-(2-fluorophenyl)-6-hydroxy-2-isopropylpyridin-3-yl)amino)nicotinate (360 mg, 98.36%) as brown solid. LCMS: (M+1)⁺: 368.3.

### Step-9: Synthesis of (S)-4-((5-(2-fluorophenyl)-2-isopropyl-6-oxo-1,6-dihydropyridin-3-yl)amino)-N-(2-hydroxypropyl)nicotinamide

To a stirred solution of lithium 4-((5-(2-fluorophenyl)-6-hydroxy-2-isopropylpyridin-3-yl)amino)nicotinate (0.180 g, 0.489 mmol, 1.0 eq) in DMF (2 mL) was added HATU (0.28 g, 0.735 mmol, 1.5 eq) at RT. The resulting mixture was stirred at RT for 5 minutes, followed by addition of (S)-1-aminopropan-2-ol (0.055 g, 0.735 mmol, 1.5 eq) and DIPEA (0.255 mL, 1.467 mmol, 3.0 eq) and stirred at RT for 1 h. Reaction mixture was diluted with water (50 mL) and extracted with ethyl acetate (2×50 mL). The combined organic extracts were washed with water (50 mL), with brine (50 mL) dried over Na₂SO₄ and concentrated under reduced and purified by combi flash chromatography [silic gel 100-200 mesh; elution 0-10% MeOH in DCM] to afford the desired compound (S)-4-((5-(2-fluorophenyl)-2-isopropyl-6-oxo-1,6-dihydropyridin-3-yl)amino)-N-(2-hydroxypropyl)nicotinamide (65 mg, 31.25%) as off white solid. LCMS: (M+1)⁺ 425.3. UPLC: At 254 nm=98.06%, At 220 nm=97.01%.

¹H NMR (400 MHz, DMSO-d₆): δ 11.79 (brs, 1H), 9.48 (brs, 1H), 8.47-8.77 (m, 2H), 8.14 (d, J=5.70 Hz, 1H), 7.52 (t, J=7.67 Hz, 1H), 7.25-7.49 (m, 2H), 6.97-7.25 (m, 2H), 6.43 (d, J=5.70 Hz, 1H), 4.78 (d, J=4.82 Hz, 1H), 3.81 (td, J=5.70, 11.40 Hz, 1H), 3.09-3.29 (m, 2H), 2.91-3.09 (m, 1H), 1.11-1.39 (m, 6H), 1.08 (d, J=6.14 Hz, 3H).

### Example-4: Synthesis of (S)-4-((5-(2-fluorophenyl)-2-isopropyl-6-oxo-1,6-dihydropyridin-3-yl)amino)-N-(2-hydroxypropyl)nicotinamide: (Compound 1.4)

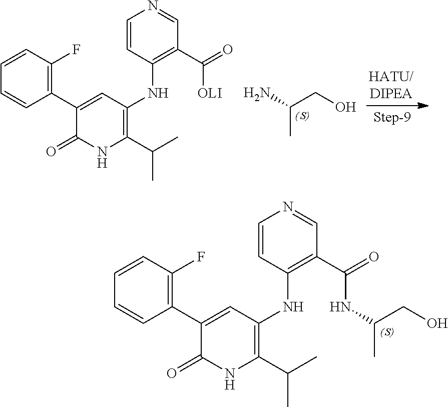

Steps 1 to 8 are the same as in Example 3.

### Step-9: Synthesis of (S)-4-((5-(2-fluorophenyl)-2-isopropyl-6-oxo-1,6-dihydropyridin-3-yl)amino)-N-(2-hydroxypropyl)nicotinamide

To a stirred solution of lithium 4-((5-(2-fluorophenyl)-6-hydroxy-2-isopropylpyridin-3-yl)amino)nicotinate (0.180 g, 0.489 mmol, 1.0 eq) in DMF (2 mL) was added HATU (0.28 g, 0.735 mmol, 1.5 eq) at RT. The resulting mixture was stirred at RT for 5 min, followed by addition of (S)-1-aminopropan-2-ol (0.055 g, 0.735 mmol, 1.5 eq) and DIPEA (0.255 mL, 1.467 mmol, 3.0 eq) and stirred at RT for 1 h. Reaction mixture was diluted with water (50 mL) and extracted with ethyl acetate (2×50 mL). The combined organic extracts were washed with water (50 mL), with brine (50 mL) dried over Na₂SO₄ and concentrated under reduced and purified by combi flash chromatography [silic gel 100-200 mesh; elution 0-10% MeOH in CH₂Cl₂] to afford the desired compound, (S)-4-((5-(2-fluorophenyl)-2-isopropyl-6-oxo-1,6-dihydropyridin-3-yl)amino)-N-(2-hydroxypropyl)nicotinamide (41 mg, 19.71%) as off white solid. LCMS: (M+1)⁺425.3. UPLC: At 254 nm: 92.77%, At 220 nm: 94.24%.

¹H NMR (400 MHz, DMSO-d₆): δ 11.79 (brs, 1H), 9.45 (brs, 1H), 8.66 (s, 1H), 8.31 (d, J=7.89 Hz, 1H), 8.13 (d, J=5.70 Hz, 1H), 7.52 (t, J=7.67 Hz, 1H), 7.35 (brs, 2H), 7.06-7.25 (m, 2H), 6.42 (d, J=5.70 Hz, 1H), 4.76 (t, J=5.70 Hz, 1H), 3.95-4.17 (m, 1H), 3.46 (dd, J=5.48, 10.74 Hz, 1H), 3.17 (d, J=5.26 Hz, 1H), 3.02 (brs, 1H), 1.14-1.42 (m, 6H), 1.13 (d, 3H).

### Example-5: Synthesis of (S)-4-((5-(5-chloro-2-fluorophenyl)-2-isopropyl-6-oxo-1,6-dihydropyridin-3-yl)amino)-N-(2-hydroxypropyl)nicotinamide: (Compound 1.5)

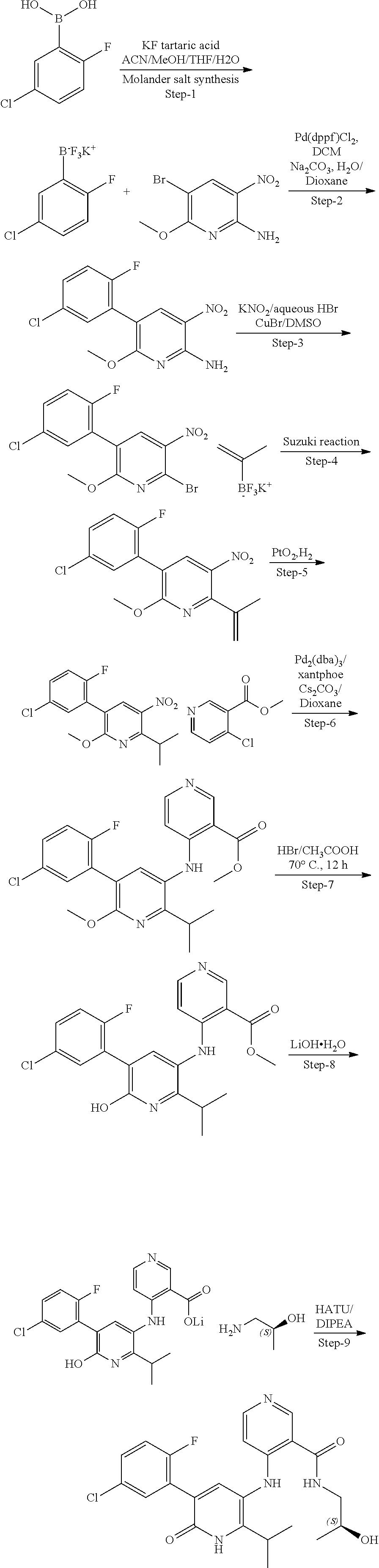

### Step-1: Synthesis of potassium (5-chloro-2-fluorophenyl)trifluoroborate

To a stirred solution of (5-chloro-2-fluorophenyl)boronic acid (5.0 g, 28.63 mmol, 1.0 eq) in MeOH (2 mL per mmol of boronic acid) and ACN (2 mL per mmol of boronic acid) at RT was added a solution of KF (6.66 g, 114.68 mmol, 4.0 eq) in water (0.1 mL per mmol) and the resulting mixture was stirred for 5 min until complete dissolution occurred. To this solution was added a solution of L-(+)-tarteric acid (8.82 g, 58.773 mmol, 2.05 eq) in THF (1.5 mL per mmol of boronic acid) dropwise and the resulting mixture was stirred at RT for 1 h. ACN (5 mL per mmol of boronic acid) was added the reaction mixture was stirred for an additional 5 min. then filtered and residue was rinsed with ACN (50 mL), filtrate was concentrated to afford the desired compound potassium (5-chloro-2-fluorophenyl)trifluoroborate (6.1 g, 89.97%) as white solid.

¹H NMR (400 MHz, DMSO-d₆): δ 7.24 (brs, 1H), 7.03-7.15 (m, 1H), 6.84 (t, J=8.33 Hz, 1H).

### Step-2: Synthesis of 5-(5-chloro-2-fluorophenyl)-6-methoxy-3-nitropyridin-2-amine

To a stirred solution of 5-bromo-6-methoxy-3-nitropyridin-2-amine (6.0 g, 24.190 mmol, 1.0 eq) and potassium (5-chloro-2-fluorophenyl)trifluoroborate (7.43 g, 31.447 mmol, 1.3 eq) in dioxane (90 mL) was added a 2M solution of Na₂CO₃ (5.128 g, 48.38 mmol, 2.0 eq) at RT. The resulting mixture was purged with nitrogen for 10 min followed by addition of Pd(dppf)Cl₂.DCM (0.987 g, 1.209 mmol, 0.05 eq) and again purged with nitrogen for 10 min. The reaction mixture was heated at 100° C. for overnight. The progress of reaction was monitored by LCMS. The reaction mixture was diluted with water (50 mL), extracted with EtOAc (2×200 mL). The combined organic layers were washed with water (10 mL), with brine (50 mL), dried over Na₂SO₄, concentrated and purified by combi flash chromatography [silica gel 100-200 mesh: elution 0-10% EtOAc in Hexane] to afford the desired compound 5-(5-chloro-2-fluorophenyl)-6-methoxy-3-nitropyridin-2-amine (2.5 mg, 34.81%) as brown solid. LCMS: (M+1)⁺: 298.1.

### Step-3: Synthesis of 2-bromo-5-(2-fluorophenyl)-6-methoxy-3-nitropyridine: (A)

48% HBr (5.418 mL, 32.316 mmol, 3.2 eq) was added to DMSO (10 mL) at 0° C. and stirred at RT. (B) To stirred solution of 5-(5-chloro-2-fluorophenyl)-6-methoxy-3-nitropyridin-2-amine (3.0 g, 10.099 mmol, 1.0 eq) in DMSO (20 mL) was added KNO₂ (4.05 g, 47.405 mmol, 4.7 eq) and CuBr (0.362 g, 2.524 mmol, 0.25 eq) at 50° C., followed by addition of a solution prepared under (A). The resulting mixture was stirred for overnight at the same temperature. The progress of reaction was monitored by ¹HNMR. The reaction mixture was basified with saturated solution of Na₂CO₃ (30 mL), extracted with diethyl ether (2×300 mL). The combined organic layers were washed with water (100 mL), with brine (50 mL), dried over Na₂SO₄, concentrated and purified by combi flash chromatography [silica gel 100-200 mesh: elution 0-10% EtOAc in Hexane] to afford the desired compound 2-bromo-5-(2-fluorophenyl)-6-methoxy-3-nitropyridine (1.0 g, 27.39%) as brown solid.

¹H NMR (400 MHz, DMSO-d₆): δ 8.53 (s, 1H), 7.65 (dd, J=2.63, 6.14 Hz, 1H), 7.59 (ddd, J=3.07, 4.28, 8.88 Hz, 1H), 7.41 (t, J=9.21 Hz, 1H), 3.99 (s, 3H).

### Step-4: Synthesis of 3-(5-chloro-2-fluorophenyl)-2-methoxy-5-nitro-6-(prop-1-en-2-yl)pyridine

To a stirred solution of 2-bromo-5-(2-fluorophenyl)-6-methoxy-3-nitropyridine (1.0 g, 2.765 mmol, 1.0 eq) and potassium trifluoro(prop-1-en-2-yl)borate (0.532 g, 3.596 mmol, 1.3 eq) in methanol (15.0 mL) was added triethylamine (0.38 mL, 2.765 mmol, 1.0 eq) rt. The resulting mixture was purged with nitrogen for 10 min followed by addition of Pd(dppf)Cl₂.DCM (0.113 g, 0.1382 mmol, 0.05 eq) and again purged with nitrogen for 10 min. The reaction mixture was heated at 100° C. for overnight. The progress of reaction was monitored by ¹HNMR. The reaction mixture was concentrated, diluted with water (50 mL), extracted with EtOAc (2×100 mL). The combined organic layers were washed with water (50 mL), with brine (50 mL), dried over Na₂SO₄, concentrated and purified by combi flash chromatography [silica gel 100-200 mesh: elution 0-20% EtOAc in Hexane] to afford the desired compound 3-(5-chloro-2-fluorophenyl)-2-methoxy-5-nitro-6-(prop-1-en-2-yl)pyridine (0.70 g, 89.26%) as yellow solid.

¹H NMR (400 MHz, DMSO-d₆): δ 8.53 (s, 1H), 7.65 (dd, J=2.63, 6.14 Hz, 1H), 7.59 (ddd, J=3.07, 4.28, 8.88 Hz, 1H), 7.41 (t, J=9.21 Hz, 1H), 3.99 (s, 3H).

### Step-5: Synthesis of 5-(5-chloro-2-fluorophenyl)-2-isopropyl-6-methoxypyridin-3-amine

To a stirred solution of 3-(5-chloro-2-fluorophenyl)-2-methoxy-5-nitro-6-(prop-1-en-2-yl)pyridine (0.70 g, 2.169 mmol, 1.0 eq) in methanol (30 mL) was added PtO₂ (100 mg) at RT. The reaction mixture was purged with hydrogen for 1 h. The progress of reaction was monitored by LCMS. The reaction mixture was filtered and the residue was washed with methanol (50 mL), filtrate was concentrated to afford the desired compound 5-(5-chloro-2-fluorophenyl)-2-isopropyl-6-methoxypyridin-3-amine (0.63 g, 98.59%) as brown solid. LCMS: (M+1)⁺: 295.2

### Step-6: Synthesis of methyl 4-((5-(5-chloro-2-fluorophenyl)-2-isopropyl-6-methoxypyridin-3-yl)amino)nicotinate

To a stirred solution of compound 5-(5-chloro-2-fluorophenyl)-2-isopropyl-6-methoxypyridin-3-amine (0.65 g, 2.205 mmol, 1.0 eq) and methyl 4-chloronicotinate (0.378 g, 2.205 mmol, 1.0 eq) in dioxane (30 mL) was added Cs₂CO₃ (2.87 g, 8.820 mmol, 4.0 eq) at RT. The resulting mixture was purged with nitrogen for 10 min followed by addition of Pd₂(dba)₃ (0.242 g, 0.264 mmol, 0.12 eq) and xantphos (0.191 g, 0.330 mmol, 0.15 eq) and again purged with nitrogen for 10 min. The reaction mixture was heated at 100° C. for overnight. The progress of reaction was monitored by LCMS. The reaction mixture was diluted with water (50 mL), extracted with EtOAc (2×100 mL). The combined organic layers were washed with water (50 mL), with brine (50 mL), dried over Na₂SO₄, concentrated and purified by combi flash chromatography [silica gel 100-200 mesh: elution 0-30% EtOAc in Hexane] to afford the desired compound methyl 4-((5-(5-chloro-2-fluorophenyl)-2-isopropyl-6-methoxypyridin-3-yl)amino)nicotinate (300 mg, 31.67%) as brown solid. LCMS: (M+1)⁺: 430.3

### Step-7: Synthesis of methyl 4-((5-(5-chloro-2-fluorophenyl)-6-hydroxy-2-isopropylpyridin-3-yl)amino)nicotinate

To a stirred solution of methyl 4-((5-(5-chloro-2-fluorophenyl)-2-isopropyl-6-methoxypyridin-3-yl)amino)nicotinate (0.300 g, 0.6978 mmol, 1.0 eq) in acetic acid (0.078 M) was added 48% aqueous HBr solution (0.35 mL, 2.0936 mmol, 3.0 eq) at RT. The reaction mixture was stirred rt for overnight. The progress of reaction was monitored by LCMS. The reaction mixture was basified with saturated aqueous solution of NaHCO₃ (50 mL), extracted with EtOAc (2×100 mL). The combined organic layers were washed with water (50 mL), with brine (50 mL), dried over Na₂SO₄, concentrated to afford the desired compound methyl 4-((5-(5-chloro-2-fluorophenyl)-6-hydroxy-2-isopropylpyridin-3-yl)amino)nicotinate (280 mg, 95.00%) as brown solid. LCMS: (M+1)⁺: 416.3.

### Step-8: Synthesis of lithium 4-((5-(5-chloro-2-fluorophenyl)-6-hydroxy-2-isopropylpyridin-3-yl)amino)nicotinate

To a stirred solution of methyl 4-((5-(5-chloro-2-fluorophenyl)-6-hydroxy-2-isopropylpyridin-3-yl)amino)nicotinate (0.280 g, 0.6733 mmol, 1.0 eq) in THF (4.0 mL) was added a solution of LiOH.H₂O (0.0565 g, 1.3466 mmol, 2.0 eq) in water (1.0 mL) at RT. The reaction mixture was stirred at for overnight. The progress of reaction was monitored by LCMS. The reaction mixture was concentrated to afford the desired lithium 4-((5-(5-chloro-2-fluorophenyl)-6-hydroxy-2-isopropylpyridin-3-yl)amino)nicotinate (265 mg, 98.14%) as brown solid. LCMS: (M+1)⁺: 402.3.

### Step-9: Synthesis of (S)-4-((5-(5-chloro-2-fluorophenyl)-2-isopropyl-6-oxo-1,6-dihydropyridin-3-yl)amino)-N-(2-hydroxypropyl)nicotinamide

To a stirred solution of lithium 4-((5-(5-chloro-2-fluorophenyl)-6-hydroxy-2-isopropylpyridin-3-yl)amino)nicotinate (0.115 g, 0.286 mmol, 1.0 eq) in DMF (2 mL) was added HATU (0.163 g, 0.429 mmol, 1.5 eq) at RT. The resulting mixture was stirred at RT for 5 minutes, followed by addition of (S)-1-aminopropan-2-ol (0.032 g, 0.429 mmol, 1.5 eq) and DIPEA (0.15 mL, 0.858 mmol, 3.0 eq) and stirred at RT for 1 h. Reaction mixture was diluted with water (50 mL) and extracted with ethyl acetate (2×50 mL). The combined organic extracts were washed with water (50 mL), with brine (50 mL) dried over Na₂SO₄ and concentrated under reduced and purified by combi flash chromatography [silic gel 100-200 mesh; elution 0-10% MeOH in DCM] to afford the desired compound (S)-4-((5-(5-chloro-2-fluorophenyl)-2-isopropyl-6-oxo-1,6-dihydropyridin-3-yl)amino)-N-(2-hydroxypropyl)nicotinamide (35 mg, 26.71%) as off white solid. LCMS: (M+1)⁺459.3. UPLC: At 254 nm=94.06%, At 220 nm=94.63%.

¹H NMR (400 MHz, DMSO-d₆): δ 11.86 (brs, 1H), 9.47 (brs, 1H), 8.68 (s, 1H), 8.62 (t, J=5.72 Hz, 1H), 8.14 (d, J=5.09 Hz, 1H), 7.64 (dd, J=2.54, 6.36 Hz, 1H), 7.37-7.51 (m, 1H), 7.30 (t, J=9.54 Hz, 1H), 6.44 (d, J=5.09 Hz, 1H), 5.76 (s, 1H), 4.77 (d, J=5.09 Hz, 1H), 3.82 (td, J=5.72, 11.44 Hz, 1H), 3.13-3.26 (m, 1H), 2.94-3.10 (m, 1H), 2.55 (s, 1H), 1.12-1.28 (m, 6H), 1.01-1.12 (m, 3H).

### Example-6: Synthesis of (S)-4-((5-(5-chloro-2-fluorophenyl)-2-isopropyl-6-oxo-1,6-dihydropyridin-3-yl)amino)-N-(1-hydroxypropyl)nicotinamide: (Compound 1.6)

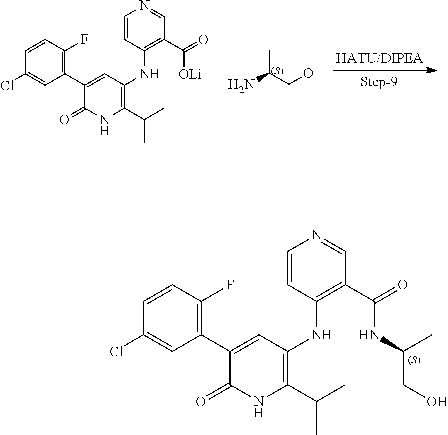

Steps 1 to 8 are the same as in Example 5.

### Step-9: Synthesis of (S)-4-((5-(5-chloro-2-fluorophenyl)-2-isopropyl-6-oxo-1,6-dihydropyridin-3-yl)amino)-N-(1-hydroxypropyl)nicotinamide

To a stirred solution of lithium 4-((5-(5-chloro-2-fluorophenyl)-6-hydroxy-2-isopropylpyridin-3-yl)amino)nicotinate (0.150 g, 0.373 mmol, 1.0 eq) in DMF (2 mL) was added HATU (0.212 g, 0.559 mmol, 1.5 eq) at RT. The resulting mixture was stirred at RT for 5 minutes, followed by addition of (S)-2-aminopropan-1-ol (0.042 g, 0.559 mmol, 1.5 eq) and DIPEA (0.194 mL, 1.119 mmol, 3.0 eq) and stirred at RT for 1 h. Reaction mixture was diluted with water (50 mL) and extracted with ethyl acetate (2×50 mL). The combined organic extracts were washed with water (50 mL), with brine (50 mL) dried over Na₂SO₄ and concentrated under reduced and purified by combi flash chromatography [silic gel 100-200 mesh; elution 0-10% MeOH in DCM] to afford the desired compound (S)-4-((5-(5-chloro-2-fluorophenyl)-2-isopropyl-6-oxo-1,6-dihydropyridin-3-yl)amino)-N-(1-hydroxypropyl)nicotinamide (50 mg, 29.29%) as off white solid. LCMS: (M+1)⁺: 459.3. UPLC: At 254 nm=93.95%, At 220 nm=94.58%.

¹H NMR (400 MHz, DMSO-d₆): δ 11.84 (brs, 1H), 9.49 (brs, 1H), 8.68 (s, 1H), 8.31 (d, J=7.63 Hz, 1H), 8.14 (d, J=5.09 Hz, 1H), 7.60-7.70 (m, 1H), 7.40-7.51 (m, 2H), 7.30 (t, J=8.90 Hz, 1H), 7.22 (brs, 1H), 6.44 (d, J=6.36 Hz, 1H), 4.75 (t, J=5.72 Hz, 1H), 3.93-4.16 (m, 1H), 3.43-3.58 (m, 1H), 3.35-3.43 (m, 1H), 2.94-3.09 (m, 1H), 1.00-1.28 (m, 9H).

### Example-7: Synthesis of (S)-4-((5-(5-chloro-2-fluorophenyl)-1-methyl-6-oxo-1,6-dihydropyridin-3-yl)amino)-N-(2-hydroxypropyl)nicotinamide: (Compound 1.7)

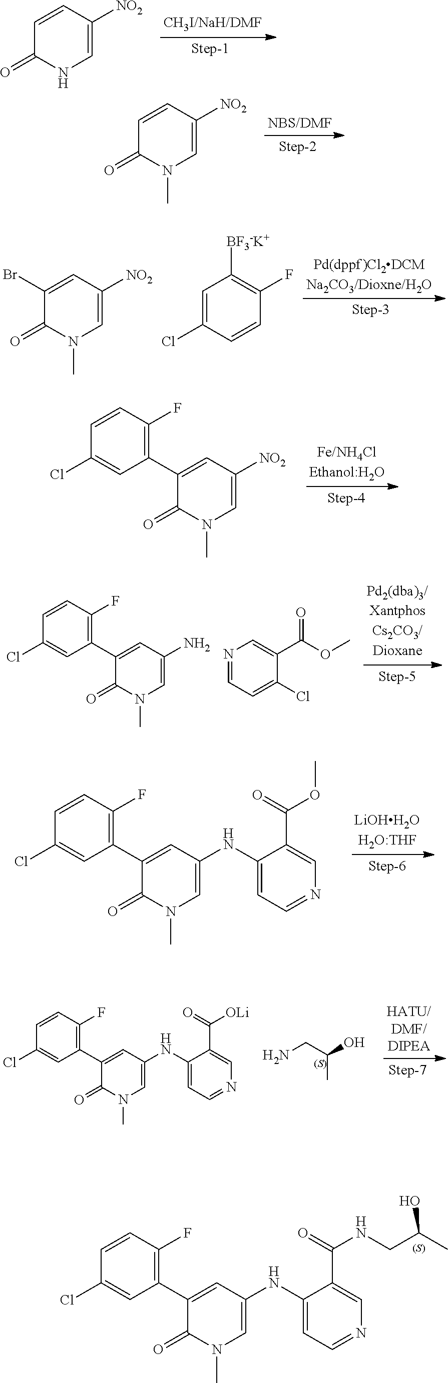

### Step-1: Synthesis of 1-methyl-5-nitropyridin-2(1H)-one

To a stirred solution of 5-nitropyridin-2(1H)-one (2.0 g, 14.275 mmol, 1.0 eq) in DMF (50 mL) was added NaH (0.686 g, 17.13 mmol, 1.2 eq) at 0° C. The reaction mixture was stirred at the same temperature for 30 min followed by dropwise addition of CH₃I (0.978 mL, 15.70 mmol). The reaction mixture was stirred at room temperature for 1 h. The progress of reaction was monitored by LCMS. The reaction mixture was diluted with saturated solution of NaHCO₃ (50 mL) and ice cold water (50 mL), the formation of precipitates was observed, filtered and washed with water (50 mL), dried under vacuum to afford the desired compound 1-methyl-5-nitropyridin-2(1H)-one (1.75 g, 79.54%) as light yellow solid. LCMS: (M+1)⁺: 155.2.

¹H NMR (400 MHz, CDCl₃): δ 8.63 (d, J=3.07 Hz, 1H), 8.10 (dd, J=2.85, 9.87 Hz, 1H), 6.56 (d, J=10.09 Hz, 1H), 3.66 (s, 3H).

### Step-2: Synthesis of 3-bromo-1-methyl-5-nitropyridin-2(1H)-one

To a stirred solution of 1-methyl-5-nitropyridin-2(1H)-one (1.75 g, 11.354 mmol, 1.0 eq) in DMF (35 mL) was added NBS (2.425 g, 13.625 mmol, 1.2 eq) at RT. The reaction mixture was stirred at RT for overnight. The progress of reaction was monitored by LCMS. The reaction mixture was poured in ice cold water (50 mL), filtered and the residue was washed with water (50 mL), dried under vacuum to afford the desired compound 3-bromo-1-methyl-5-nitropyridin-2(1H)-one (2.1 g, 79.24%) as light yellow solid. LCMS: (M+1)⁺: 233.1

¹H NMR (400 MHz, CDCl₃): δ 8.63 (d, J=2.19 Hz, 1H), 8.52 (d, J=2.63 Hz, 1H), 3.73 (s, 4H).

### Step-3: Synthesis of 3-(5-chloro-2-fluorophenyl)-1-methyl-5-nitropyridin-2(1H)-one

To a stirred solution of 3-bromo-1-methyl-5-nitropyridin-2(1H)-one (2.1 g, 9.012 mmol, 1.0 eq) and potassium (5-chloro-2-fluorophenyl)trifluoroborate (3.2 g, 13.518 mmol, 1.5 eq) in dioxane (50 mL) was added a 2M solution of Na₂CO₃ (1.91 g, 18.024 mmol, 2.0 eq) in H₂O at RT. The resulting mixture was purged with nitrogen for 10 min followed by addition of Pd(dppf)Cl₂.DCM (0.368 g, 0.450 mmol, 0.05 eq), again purged with nitrogen for 10 min. The reaction mixture was heated at 100° C. for overnight. The progress of reaction was monitored by LCMS. The reaction mixture was diluted with water (50 mL), extracted with EtOAc (2×100 mL). The combined organic layers were washed with water (50 mL), with brine (50 mL), dried over Na₂SO₄, concentrated and purified by combi flash chromatography [silica gel 100-200 mesh: elution 0-30% EtOAc in Hexane] to afford the desired compound 3-(5-chloro-2-fluorophenyl)-1-methyl-5-nitropyridin-2(1H)-one (1.6 g, 62.99%) as off white solid. LCMS: (M+1)⁺: 283.2.

### Step-4: Synthesis of 5-amino-3-(5-chloro-2-fluorophenyl)-1-methylpyridin-2(1H)-one

To a stirred solution of 3-(5-chloro-2-fluorophenyl)-1-methyl-5-nitropyridin-2(1H)-one (1.6 g, 5.660 mmol, 1.0 eq) in EtOH (25 mL) was added Fe (2.53 g, 45.285 mmol, 8.0 eq) and a solution of NH₄Cl (3.03 g, 56.60 mmol, 10.0 eq) at RT. The resulting mixture was heated at 90° C. for 60 min. The progress of reaction was monitored by LCMS. The reaction mixture was filtered through celite the residue was washed with EtOH (50 mL) the filtrate was concentrated and the residue was dissolved in EtOAc (200 mL), washed with water (2×100 mL), dried over Na₂SO₄, and concentrated to afford the desired 5-amino-3-(5-chloro-2-fluorophenyl)-1-methylpyridin-2(1H)-one (1.1 g, 76.92%) as green solid. LCMS: (M+1)⁺: 253.2

### Step-5: Synthesis of methyl 4-((5-(5-chloro-2-fluorophenyl)-1-methyl-6-oxo-1,6-dihydropyridin-3-yl)amino)nicotinate

To a stirred solution of compound 5-amino-3-(5-chloro-2-fluorophenyl)-1-methylpyridin-2(1H)-one (1.0 g, 3.958 mmol, 1.0 eq) and methyl 4-chloronicotinate (0.747 g, 4.353 mmol, 1.0 eq) in dioxane (50 mL) was added Cs₂CO₃ (5.16 g, 15.832 mmol, 4.0 eq) at RT. The resulting mixture was purged with nitrogen for 10 min followed by addition of Pd₂(dba)₃ (0.432 g, 0.475 mmol, 0.12 eq) and xantphos (0.342 g, 0.593 mmol, 0.15 eq) and again purged with nitrogen for 10 min. The reaction mixture was heated at 100° C. for overnight. The progress of reaction was monitored by LCMS. The reaction mixture was diluted with water (50 mL), extracted with EtOAc (2×100 mL). The combined organic layers were washed with water (50 mL), with brine (50 mL), dried over Na₂SO₄, concentrated and purified by combi flash chromatography [silica gel 100-200 mesh: elution 0-30% EtOAc in Hexane] to afford the desired compound methyl 4-((5-(5-chloro-2-fluorophenyl)-1-methyl-6-oxo-1,6-dihydropyridin-3-yl)amino)nicotinate (350 mg, 23.33%) as brown solid. LCMS: (M+1)⁺: 388.3

### Step-6: Synthesis of lithium 4-((5-(5-chloro-2-fluorophenyl)-1-methyl-6-oxo-1,6-dihydropyridin-3-yl)amino)nicotinate

To a stirred solution of methyl 4-((5-(5-chloro-2-fluorophenyl)-1-methyl-6-oxo-1,6-dihydropyridin-3-yl)amino)nicotinate (0.200 g, mmol, 1.0 eq) in THF (4.0 mL) was added a solution of LiOH.H₂O (0.043 g, 1.031 mmol, 2.0 eq) in water (1.0 mL) at RT. The reaction mixture was stirred at for overnight. The progress of reaction was monitored by LCMS. The reaction mixture was concentrated to afford the desired lithium 4-((5-(5-chloro-2-fluorophenyl)-1-methyl-6-oxo-1,6-dihydropyridin-3-yl)amino)nicotinate (192 mg, 98.46%) as brown solid. LCMS: (M+1)⁺: 374.2

### Step-7: Synthesis of (S)-4-((5-(5-chloro-2-fluorophenyl)-1-methyl-6-oxo-1,6-dihydropyridin-3-yl)amino)-N-(2-hydroxypropyl)nicotinamide

To a stirred solution of lithium 4-((5-(5-chloro-2-fluorophenyl)-1-methyl-6-oxo-1,6-dihydropyridin-3-yl)amino)nicotinate (0.120 g, 0.316 mmol, 1.0 eq) in DMF (2 mL) was added HATU (0.180 g, 0.475 mmol, 1.5 eq) at RT. The resulting mixture was stirred at RT for 5 minutes, followed by addition of (S)-1-aminopropan-2-ol (0.035 g, 0.475 mmol, 1.5 eq) and DIPEA (0.165 mL, 0.948 mmol, 3.0 eq) and stirred at RT for 30 min. Reaction mixture was diluted with water (50 mL) and extracted with ethyl acetate (2×50 mL). The combined organic extracts were washed with water (50 mL), with brine (50 mL) dried over Na₂SO₄ and concentrated under reduced and purified by combi flash chromatography [silic gel 100-200 mesh; elution 0-10% MeOH in DCM] to afford the desired compound (S)-4-((5-(5-chloro-2-fluorophenyl)-1-methyl-6-oxo-1,6-dihydropyridin-3-yl)amino)-N-(2-hydroxypropyl)nicotinamide (55 mg, 40.44%) as light yellow solid. LCMS: (M+1)⁺: 431.3, UPLC: At 254 nm=99.29%, At 220 nm=99.21%.

¹H NMR (400 MHz, DMSO-d₆): δ 9.64 (s, 1H), 8.68 (s, 1H), 8.63 (t, J=5.26 Hz, 1H), 8.16 (d, J=5.70 Hz, 1H), 7.93 (d, J=2.63 Hz, 1H), 7.60 (dd, J=2.85, 6.36 Hz, 1H), 7.56 (d, J=2.19 Hz, 1H), 7.45 (ddd, J=2.85, 4.28, 8.66 Hz, 1H), 7.24-7.34 (m, 1H), 6.70 (d, J=5.70 Hz, 1H), 4.77 (d, J=4.82 Hz, 1H), 3.81 (td, J=5.70, 11.40 Hz, 1H), 3.50 (s, 3H), 3.14-3.26 (m, 2H), 1.08 (d, J=6.14 Hz, 3H).

### Example-8: Synthesis of (S)-4-((5-(5-chloro-2-fluorophenyl)-1-methyl-6-oxo-1,6-dihydropyridin-3-yl)amino)-N-(1-hydroxypropan-2-yl)nicotinamide: (Compound 1.8)

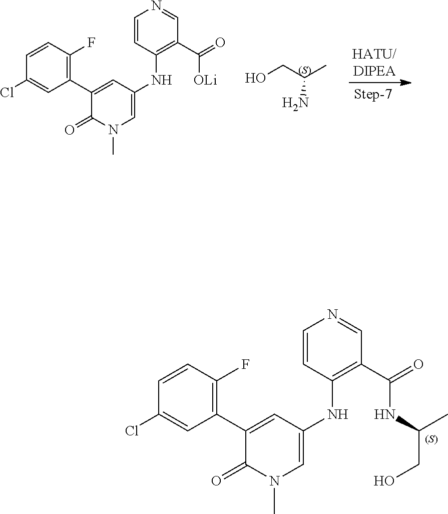

Steps 1 to 6 are the same as in Example 7.

### Step-7: Synthesis of (S)-4-((5-(5-chloro-2-fluorophenyl)-1-methyl-6-oxo-1,6-dihydropyridin-3-yl)amino)-N-(1-hydroxypropan-2-yl)nicotinamide

To a stirred solution of lithium 4-((5-(5-chloro-2-fluorophenyl)-1-methyl-6-oxo-1,6-dihydropyridin-3-yl)amino)nicotinate (0.100 g, 0.2633 mmol, 1.0 eq) in DMF (2 mL) was added HATU (0.15 g, 0.395 mmol, 1.5 eq) at RT. The resulting mixture was stirred at RT for 5 minutes, followed by addition of (S)-2-aminopropan-1-ol (0.030 g, 0.395 mmol, 1.5 eq) and DIPEA (0.137 mL, 0.7899 mmol, 3.0 eq) and stirred at RT for 30 min. Reaction mixture was diluted with water (50 mL) and extracted with ethyl acetate (2×50 mL). The combined organic extracts were washed with water (50 mL), with brine (50 mL) dried over Na₂SO₄ and concentrated under reduced and purified by combi flash chromatography [silic gel 100-200 mesh; elution 0-10% MeOH in DCM] to afford the desired compound (S)-4-((5-(5-chloro-2-fluorophenyl)-1-methyl-6-oxo-1,6-dihydropyridin-3-yl)amino)-N-(1-hydroxypropan-2-yl)nicotinamide (55 mg, 48.67%) as off white solid. LCMS: (M+1)⁺: 431.3. UPLC: At 254 nm=96.89%, At 220 nm=99.10%.

¹H NMR (400 MHz, DMSO-d₆): δ 9.64 (s, 1H), 8.67 (s, 1H), 8.32 (d, J=7.89 Hz, 1H), 8.16 (d, J=5.70 Hz, 1H), 7.94 (d, J=2.63 Hz, 1H), 7.60-7.67 (m, 1H), 7.53-7.60 (m, 1H), 7.41-7.48 (m, 1H), 7.30 (t, J=9.21 Hz, 1H), 6.70 (d, J=6.14 Hz, 1H), 4.75 (t, J=5.92 Hz, 1H), 3.98-4.11 (m, 1H), 3.48-3.54 (m, 3H), 3.41-3.48 (m, 1H), 3.34-3.41 (m, 1H), 1.14 (d, J=6.58 Hz, 3H).

### Example-9: Synthesis of (S)-4-((5-(5-chloro-2-fluorophenyl)-2-(dimethylamino)-6-hydroxypyridin-3-yl)amino)-N-(2-hydroxypropyl)nicotinamide: (Compound 1.9)

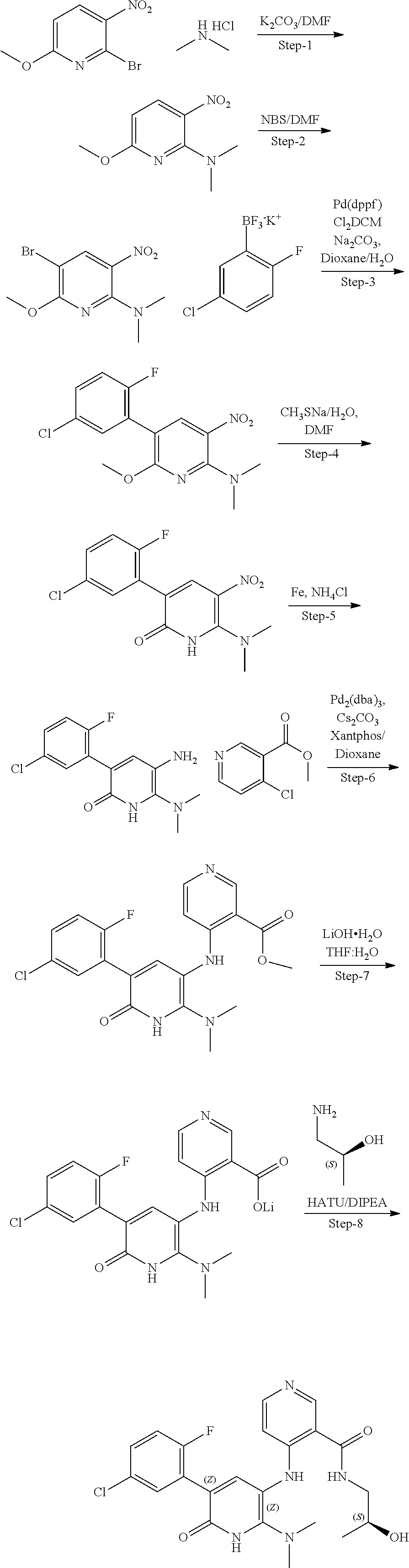

### Step-1: Synthesis of 6-methoxy-N,N-dimethyl-3-nitropyridin-2-amine

To a stirred solution of 2-bromo-6-methoxy-3-nitropyridine (2.0 g, 8.583 mmol, 1.0 eq) and dimethylamine hydrochloride (0.840 g, 10.299 mmol, 1.2 eq) in DMF (20 mL) was added K₂CO₃ (3.56 g, 25.799 mmol, 3.0 eq) at RTrt. The reaction mixture was stirred at 80° C. for overnight. The progress of reaction was monitored by LCMS. The reaction mixture was poured in ice cold water (50 mL), the formation of precipitates was observed, filtered and washed with water (50 mL), dried under vacuum to afford the desired compound 6-methoxy-N,N-dimethyl-3-nitropyridin-2-amine (1.61 g, 96.98%) as yellow solid. LCMS: (M+1)⁺: 198.3

### Step-2:-Synthesis of 5-bromo-6-methoxy-N,N-dimethyl-3-nitropyridin-2-amine

To a stirred solution of 6-methoxy-N,N-dimethyl-3-nitropyridin-2-amine (1.60 g, 8.114 mmol, 1.0 eq) in DMF (20 mL) was added NBS (1.58 g, 8.925 mmol, 1.1 eq) at RT. The reaction mixture was stirred at RT for 1 h. The progress of reaction was monitored by LCMS. The reaction mixture was poured in ice cold water (50 mL), filtered and the residue was washed with water (50 mL), dried under vacuum to afford the desired compound 5-bromo-6-methoxy-N,N-dimethyl-3-nitropyridin-2-amine (2.09 g, 93.30%) as yellow solid. LCMS: (M+1)⁺: 276.1

### Step-3:-Synthesis of 5-(5-chloro-2-fluorophenyl)-6-methoxy-N,N-dimethyl-3-nitropyridin-2-amine

To a stirred solution of 5-bromo-6-methoxy-N,N-dimethyl-3-nitropyridin-2-amine (2.0 g, 7.244 mmol, 1.0 eq) and potassium (5-chloro-2-fluorophenyl)trifluoroborate (2.23 g, 9.417 mmol, 1.3 eq) in dioxane (50 mL) was added a 2M solution of Na₂CO₃ (1.54 g, 14.488 mmol, 2.0 eq) in H₂O (7.25 mL) at RT. The resulting mixture was purged with nitrogen for 10 min followed by addition of Pd(dppf)Cl₂.DCM (0.295 g, 0.363 mmol, 0.05 eq), again purged with nitrogen for 10 min. The reaction mixture was heated at 100° C. for overnight. The progress of reaction was monitored by LCMS. The reaction mixture was diluted with water (50 mL), extracted with EtOAc (2×100 mL). The combined organic layers were washed with water (50 mL), with brine (50 mL), dried over Na₂SO₄, concentrated and purified by combi flash chromatography [silica gel 100-200 mesh: elution 0-30% EtOAc in Hexane] to afford the desired compound 5-(5-chloro-2-fluorophenyl)-6-methoxy-N,N-dimethyl-3-nitropyridin-2-amine (2.0 g, 86.95%) as yellow solid. LCMS: (M+1)⁺: 326.2.

### Step-4:-Synthesis of 3-(5-chloro-2-fluorophenyl)-6-(dimethylamino)-5-nitropyridin-2-ol

To a stirred solution of 5-(5-chloro-2-fluorophenyl)-6-methoxy-N,N-dimethyl-3-nitropyridin-2-amine (2.0 g, 6.140 mmol, 1.0 eq) in DMF (18 mL) was added 21% solution of CH₃SNa (1.72 g, 24.560 mmol, 4.0 eq) in water (8.2 mL) at RTrt. The reaction mixture was stirred at 80° C. for overnight. The progress of reaction was monitored by LCMS. The reaction mixture was acidified with 1N HCl (5.0 mL). The reaction mixture extracted with DCM (2×50 mL). The combined organic layers were washed with water (50 mL), with brine (50 mL), dried over Na₂SO₄, concentrated and purified by combi flash chromatography [silica gel 100-200 mesh: elution 0-5.0% MeOH in DCM] to afford the desired compound 3-(5-chloro-2-fluorophenyl)-6-(dimethylamino)-5-nitropyridin-2-ol (0.840 g, 44.21%) as yellow solid. LCMS: (M+1)⁺: 312.2.

### Step-5: Synthesis of 5-amino-3-(5-chloro-2-fluorophenyl)-6-(dimethylamino)pyridin-2-ol

To a stirred solution of 3-(5-chloro-2-fluorophenyl)-6-(dimethylamino)-5-nitropyridin-2-ol (1.4 g, 4.491 mmol, 1.0 eq) in EtOH (25 mL) was added Fe (2.0 g, 35.931 mmol, 8.0 eq) and a solution of NH₄Cl (2.4 g, 44.91 mmol, 10.0 eq) at RT. The resulting mixture was heated at 90° C. for 60 min. The progress of reaction was monitored by LCMS. The reaction mixture was filtered through celite the residue was washed with EtOH (50 mL) the filtrate was concentrated and the residue was dissolved in EtOAc (200 mL), washed with water (2×100 mL), dried over Na₂SO₄, and concentrated to afford the desired 5-amino-3-(5-chloro-2-fluorophenyl)-6-(dimethylamino)pyridin-2-ol (1.145 g, 90.38%) as brown solid. LCMS: (M+1)⁺: 282.2.

### Step-6: Synthesis of methyl 4-((5-(5-chloro-2-fluorophenyl)-2-(dimethylamino)-6-hydroxypyridin-3-yl)amino)nicotinate

To a stirred solution of compound 5-amino-3-(5-chloro-2-fluorophenyl)-6-(dimethylamino)pyridin-2-ol (1.14 g, 4.046 mmol, 1.0 eq) and methyl 4-chloronicotinate (0.764 g, 4.451 mmol, 1.0 eq) in dioxane (30 mL) was added Cs₂CO₃ (5.27 g, 16.184 mmol, 4.0 eq) at RT. The resulting mixture was purged with nitrogen for 10 min followed by addition of Pd₂(dba)₃ (0.455 g, 0.497 mmol, 0.12 eq) and xantphos (0.350 g, 0.606 mmol, 0.15 eq), again purged with nitrogen for 10 min. The reaction mixture was heated at 100° C. for overnight. The progress of reaction was monitored by LCMS. The reaction mixture was diluted with water (50 mL), extracted with EtOAc (2×100 mL). The combined organic layers were washed with water (50 mL), with brine (50 mL), dried over Na₂SO₄, concentrated and purified by combi flash chromatography [silica gel 100-200 mesh: elution 0-30% EtOAc in Hexane] to afford the desired compound methyl 4-((5-(5-chloro-2-fluorophenyl)-2-(dimethylamino)-6-hydroxypyridin-3-yl)amino)nicotinate (500 mg, 29.76%) as brown solid. LCMS: (M+1)⁺: 417.2.

### Step-7: Synthesis lithium 4-((5-(5-chloro-2-fluorophenyl)-2-(dimethylamino)-6-hydroxypyridin-3-yl)amino)nicotinate

To a stirred solution of methyl 4-((5-(5-chloro-2-fluorophenyl)-2-(dimethylamino)-6-hydroxypyridin-3-yl)amino)nicotinate (0.500 g, 1.199 mmol, 1.0 eq) in THF (4.0 mL) was added a solution of LiOH.H₂O (0.10 g, 2.399 mmol, 2.0 eq) in water (1.0 mL) at RT. The reaction mixture was stirred at for overnight. The progress of reaction was monitored by LCMS. The reaction mixture was concentrated to afford the desired lithium 4-((5-(5-chloro-2-fluorophenyl)-2-(dimethylamino)-6-hydroxypyridin-3-yl)amino)nicotinate (480 mg, 99.58%) as brown solid. LCMS: (M+1)⁺: 403.2.

### Step-8: Synthesis of (S)-4-((5-(5-chloro-2-fluorophenyl)-2-(dimethylamino)-6-hydroxypyridin-3-yl)amino)-N-(2-hydroxypropyl)nicotinamide

To a stirred solution of lithium 4-((5-(5-chloro-2-fluorophenyl)-2-(dimethylamino)-6-hydroxypyridin-3-yl)amino)nicotinate (0.15 g, 0.366 mmol, 1.0 eq) in DMF (3 mL) was added HATU (0.21 g, 0.550 mmol, 1.5 eq) at RT. The resulting mixture was stirred at RT for 5 minutes, followed by addition of (S)-1-aminopropan-2-ol (0.042 g, 0.55 mmol, 1.5 eq) and DIPEA (0.191 mL, 1.1 mmol, 3.0 eq) and stirred at RT for 30 min. Reaction mixture was diluted with water (50 mL) and extracted with ethyl acetate (2×50 mL). The combined organic extracts were washed with water (50 mL), with brine (50 mL) dried over Na₂SO₄ and concentrated under reduced and purified by combi flash chromatography [silic gel 100-200 mesh; elution 0-10% MeOH in DCM] to afford the desired compound (S)-4-((5-(5-chloro-2-fluorophenyl)-2-(dimethylamino)-6-hydroxypyridin-3-yl)amino)-N-(2-hydroxypropyl)nicotinamide (29 mg, 21.32%) as light yellow solid. LCMS: (M+1)⁺: 460.2, UPLC: At 254 nm=92.36%, At 220 nm=91.63%.

¹H NMR (400 MHz, DMSO-d₆): δ 10.84 (brs, 1H), 9.68 (brs, 1H), 8.68 (s, 2H), 8.15 (d, J=6.14 Hz, 1H), 7.54 (brs, 1H), 7.38 (s, 2H), 7.26 (t, J=9.43 Hz, 1H), 6.53 (d, J=6.14 Hz, 1H), 4.78 (d, J=4.82 Hz, 1H), 3.80 (brs, 1H), 3.11-3.24 (m, 2H), 2.89 (s, 6H), 1.25 (d, J=5.70 Hz, 1H), 1.08 (d, J=6.14 Hz, 3H).

### Example-10: Synthesis of (S)-4-((5-(5-chloro-2-fluorophenyl)-2-(dimethylamino)-6-hydroxypyridin-3-yl)amino)-N-(1-hydroxypropan-2-yl)nicotinamide: (Compound 1.10)

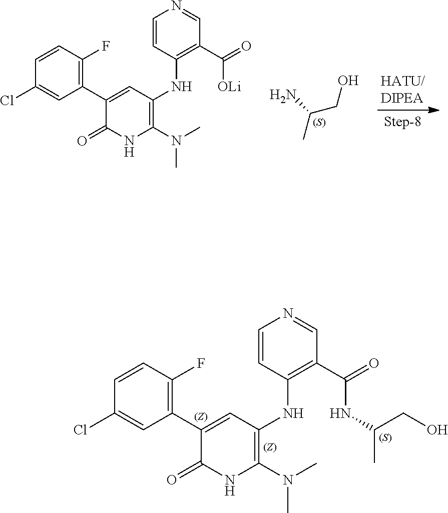

Steps 1 to 7 are the same as in Example 9.

### Step-8: Synthesis of (S)-4-((5-(5-chloro-2-fluorophenyl)-2-(dimethylamino)-6-hydroxypyridin-3-yl)amino)-N-(1-hydroxypropan-2-yl)nicotinamide

To a stirred solution of lithium 4-((5-(5-chloro-2-fluorophenyl)-2-(dimethylamino)-6-hydroxypyridin-3-yl)amino)nicotinate (0.15 g, 0.366 mmol, 1.0 eq) in DMF (3 mL) was added HATU (0.21 g, 0.550 mmol, 1.5 eq) at RT. The resulting mixture was stirred at RT for 5 minutes, followed by addition of (S)-2-aminopropan-1-ol (0.042 g, 0.55 mmol, 1.5 eq) and DIPEA (0.191 mL, 1.1 mmol, 3.0 eq) and stirred at RT for 30 min. Reaction mixture was diluted with water (50 mL) and extracted with ethyl acetate (2×50 mL). The combined organic extracts were washed with water (50 mL), with brine (50 mL) dried over Na₂SO₄ and concentrated under reduced and purified by combi flash chromatography [silic gel 100-200 mesh; elution 0-10% MeOH in DCM] to afford the desired compound (S)-4-((5-(5-chloro-2-fluorophenyl)-2-(dimethylamino)-6-hydroxypyridin-3-yl)amino)-N-(1-hydroxypropan-2-yl)nicotinamide (20 mg, 12.12%) as light yellow solid. LCMS: (M+1)⁺: 460.2, UPLC: At 254 nm=92.90%, At 220 nm=92.80%.

¹H NMR (400 MHz, DMSO-d₆): δ 10.80 (brs., 1H), 9.54 (brs, 1H), 8.65 (s, 1H), 8.33 (d, J=7.89 Hz, 1H), 8.13 (d, J=5.70 Hz, 1H), 7.54 (brs, 1H), 7.37 (brs, 2H), 7.26 (t, J=9.21 Hz, 1H), 6.50 (d, J=5.70 Hz, 1H), 4.75 (t, J=5.70 Hz, 1H), 4.03 (d, J=7.02 Hz, 1H), 3.39-3.52 (m, 1H), 2.89 (s, 6H), 1.13 (d, J=6.58 Hz, 3H).

### Example-11: Synthesis of (S)-4-((5-(5-chloro-2-fluorophenyl)-2-(dimethylamino)-6-hydroxypyridin-3-yl)amino)-N-(1-hydroxypropan-2-yl)nicotinamide: (Compound 1.11)

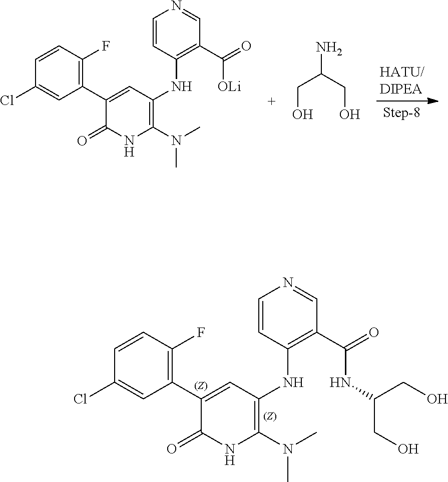

Steps 1 to 7 are the same as in Example 9.

### Step-8: Synthesis of (S)-4-((5-(5-chloro-2-fluorophenyl)-2-(dimethylamino)-6-hydroxypyridin-3-yl)amino)-N-(1-hydroxypropan-2-yl)nicotinamide

To a stirred solution of lithium 4-((5-(5-chloro-2-fluorophenyl)-2-(dimethylamino)-6-hydroxypyridin-3-yl)amino)nicotinate (0.200 g, 0.489 mmol, 1.0 eq) in DMF (3 mL) was added HATU (0.279 g, 0.733 mmol, 1.5 eq) at RT. The resulting mixture was stirred at RT for 5 minutes, followed by addition of 2-aminopropane-1,3-diol (0.067 g, 0.733 mmol, 1.5 eq) and DIPEA (0.17 mL, 0.978 mmol, 2.0 eq) and stirred at RT for 30 min. Reaction mixture was diluted with water (50 mL) and extracted with ethyl acetate (2×50 mL). The combined organic extracts were washed with water (50 mL), with brine (50 mL) dried over Na₂SO₄ and concentrated under reduced and purified by combi flash chromatography [silic gel 100-200 mesh; elution 0-10% MeOH in DCM] to afford the desired compound (S)-4-((5-(5-chloro-2-fluorophenyl)-2-(dimethylamino)-6-hydroxypyridin-3-yl)amino)-N-(1-hydroxypropan-2-yl)nicotinamide (20 mg, 8.58%) as light yellow solid. LCMS: (M+1)⁺: 476.3, UPLC: At 254 nm=84.49%, At 220 nm=83.04%.

¹H NMR (400 MHz, DMSO-d₆): δ 10.79 (brs, 1H), 9.51 (brs, 1H), 8.69 (s, 1H), 8.21 (d, J=8.33 Hz, 1H), 8.13 (d, J=6.14 Hz, 1H), 7.55 (brs, 1H), 7.32-7.40 (m, 2H), 7.26 (t, J=9.21 Hz, 1H), 6.50 (d, J=6.14 Hz, 1H), 4.68 (t, J=5.48 Hz, 2H), 3.99 (d, J=7.02 Hz, 1H), 3.52 (d, J=3.07 Hz, 4H), 2.83-2.94 (m, 6H).

### Example-12: Synthesis of tert-butyl 4-((5-(5-chloro-2-fluorophenyl)-2-(dimethylamino)-6-hydroxypyridin-3-yl)amino)-1H-pyrrolo[2,3-b]pyridine-1-carboxylate: (Compound 1.12)

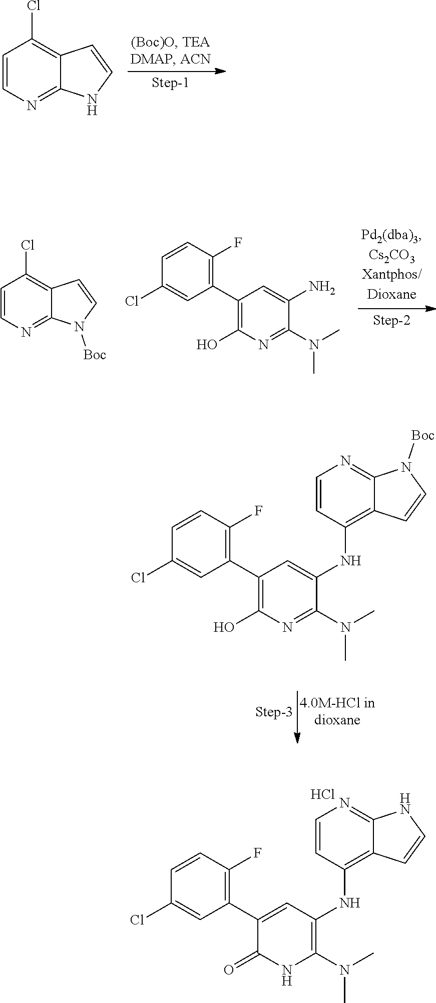

### Step-1: Synthesis of tert-butyl 4-chloro-1H-pyrrolo[2,3-b]pyridine-1-carboxylate

To a stirred solution of 4-chloro-1H-pyrrolo[2,3-b]pyridine (2.0 g, 13.107 mmol, 1.0 eq) in ACN (30 mL) was added triethylamine (3.98 g, 39.321 mmol, 3.0 eq) and DMAP (0.320 g, 2.621 mmol, 0.2 eq) at RT. The resulting mixture was allowed to cool to 0° C. followed by addition of (Boc)₂O (3.15 g, 14.418 mmol, 1.1 eq). The resulting mixture was stirred at RT for overnight. The progress of reaction was monitored by LCMS. The reaction mixture was diluted with EtOAc (50 mL), washed with water (2×100 mL), dried over Na₂SO₄, concentrated and purified by combiflash chromatography [silica gel 100-200 mesh elution 0-10% EtOAc in Hexane] to afford the desired tert-butyl 4-chloro-1H-pyrrolo[2,3-b]pyridine-1-carboxylate (2.50 g, 75.75%) as light yellow solid. LCMS: (M+1)⁺: 253.1

### Step-2: Synthesis of tert-butyl 4-((5-(5-chloro-2-fluorophenyl)-2-(dimethylamino)-6-hydroxypyridin-3-yl)amino)-1H-pyrrolo[2,3-b]pyridine-1-carboxylate

To a stirred solution of compound 5-amino-3-(5-chloro-2-fluorophenyl)-6-(dimethylamino)pyridin-2-ol (0.60 g, 2.129 mmol, 1.0 eq) and tert-butyl 4-chloro-1H-pyrrolo[2,3-b]pyridine-1-carboxylate (0.592 g, 2.342 mmol, 1.0 eq) in dioxane (30 mL) was added Cs₂CO₃ (2.77 g, 8.516 mmol, 4.0 eq) at RT. The resulting mixture was purged with nitrogen for 10 min followed by addition of Pd₂(dba)₃ (0.234 g, 0.255 mmol, 0.12 eq) and xantphos (0.185 g, 0.319 mmol, 0.15 eq) and again purged with nitrogen for 10 min. The reaction mixture was heated at 100° C. for overnight. The progress of reaction was monitored by LCMS. The reaction mixture was diluted with water (50 mL), extracted with EtOAc (2×100 mL). The combined organic layers were washed with water (50 mL), with brine (50 mL), dried over Na₂SO₄, concentrated and purified by combi flash chromatography [silica gel 100-200 mesh: elution 0-80% EtOAc in Hexane] to afford the desired compound tert-butyl 4-((5-(5-chloro-2-fluorophenyl)-2-(dimethylamino)-6-hydroxypyridin-3-yl)amino)-1H-pyrrolo[2,3-b]pyridine-1-carboxylate (250 mg, 28.28%) as brown solid. LCMS: (M+1)⁺: 498.3

### Step-3: Synthesis of 5-((1H-pyrrolo[2,3-b]pyridin-4-yl)amino)-3-(5-chloro-2-fluorophenyl)-6-(dimethylamino)pyridin-2-ol hydrochloride

tert-butyl 4-((5-(5-chloro-2-fluorophenyl)-2-(dimethylamino)-6-hydroxypyridin-3-yl)amino)-1H-pyrrolo [2,3-b]pyridine-1-carboxylate (100 mg, 0.20 mmol, 1.0 eq) was dissolved in 4.0 M-HCl in dioxane (1.5 mL) and allowed to stir at RT for 1 h. After completion of reaction, the reaction mixture was filtered and dried under reduced pressure to afford the desired compound 5-((1H-pyrrolo[2,3-b]pyridin-4-yl)amino)-3-(5-chloro-2-fluorophenyl)-6-(dimethylamino)pyridin-2-ol hydrochloride (78 mg, 89.65%) as light yellow solid. LCMS: (M+1)⁺: 398.2; UPLC: At 254 nm=93.15% and At 220 nm=94.45%.

¹H NMR (400 MHz, DMSO-d₆): δ 13.99 (brs, 1H), 12.35 (brs, 1H), 9.91 (brs, 1H), 7.96 (d, J=6.58 Hz, 1H), 7.53 (brs, 1H), 7.46 (s, 1H), 7.37 (brs, 2H), 7.25-7.30 (m, 1H), 7.04 (brs, 1H), 6.21 (brs, 1H), 2.93 (s, 6H).

### Example-13: Synthesis of 5′-((1H-pyrrolo[2,3-b]pyridin-4-yl)amino)-6′-(dimethylamino)-6-methyl-[2,3′-bipyridin]-2′(1′H)-one: (Compound 1.13)

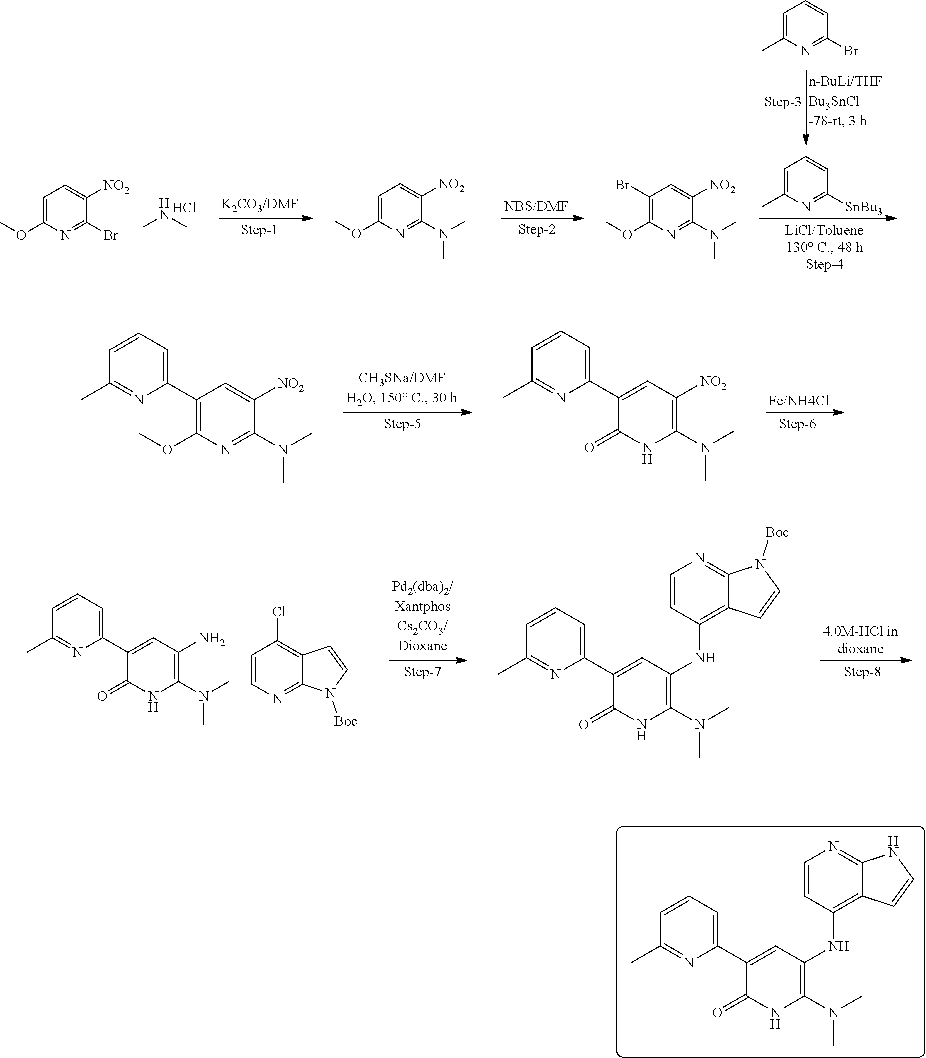

### Step-1: Synthesis of 6-methoxy-N,N-dimethyl-3-nitropyridin-2-amine

To a stirred solution of 2-bromo-6-methoxy-3-nitropyridine (5.50 g, 23.603 mmol, 1.0 eq) and dimethylamine hydrochloride (2.30 g, 28.323 mmol, 1.2 eq) in DMF (20 mL) was added K₂CO₃ (9.7 g, 70.80 mmol, 3.0 eq) at RTrt. The reaction mixture was stirred at 120° C. for 3 h. The progress of reaction was monitored by LCMS. The reaction mixture was poured in ice cold water (50 mL), the formation of precipitates was observed, filtered and washed with water (50 mL), dried under vacuum to afford the desired compound 6-methoxy-N,N-dimethyl-3-nitropyridin-2-amine (4.50 g, 96.77%) as yellow solid. LCMS: (M+1)⁺: 198.3

### Step-2:-Synthesis of 5-bromo-6-methoxy-N,N-dimethyl-3-nitropyridin-2-amine

To a stirred solution of 6-methoxy-N,N-dimethyl-3-nitropyridin-2-amine (4.50 g, 22.82 mmol, 1.0 eq) in DMF (20 mL) was added NBS (4.46 g, 25.102 mmol, 1.1 eq) at RT. The reaction mixture was stirred at RT for 1 h. The progress of reaction was monitored by LCMS. The reaction mixture was poured in ice cold water (50 mL), filtered and the residue was washed with water (50 mL), dried under vacuum to afford the desired compound 5-bromo-6-methoxy-N,N-dimethyl-3-nitropyridin-2-amine (6.10 g, 96.82%) as yellow solid. LCMS: (M+1)⁺: 276.1

### Step-3: Synthesis of 2-methyl-6-(tributylstannyl)pyridine

To a stirred solution of 2-bromo-6-methylpyridine (1.0 g, 5.813 mmol, 1.0 eq) in THF (20 mL) was dropwise added n-BuLi (3.63 mL, 5.813 mmol, 1.0 eq) at −78° C. The reaction mixture was stirred at the same temperature for 3 h, followed by addition of Bu₃SnCl (1.58 mL, 5.813 mmol, 1.0 eq). The reaction mixture was allowed to warm to rt. The progress of reaction was monitored by ¹HNMR. The reaction mixture was quenched with saturated solution of NH₄Cl (50 mL), extracted with EtOAc (2×50 mL), the combined organic layers were washed with water (50 mL), and with brine (50 mL) dried over Na₂SO₄, concentrated under vacuum to afford the desired compound 2-methyl-6-(tributylstannyl)pyridine (2.15 g, 96.75%) as yellow liquid.

¹H NMR (400 MHz, CDCl₃): δ 7.39 (t, 1H, J=7.5 Hz), 7.20 (d, 1H, J=7.5 Hz), 6.98 (dd, 1H, J=7.9, 1.1 Hz), 2.57 (s, 3H), 1.64-1.54 (m, 6H), 1.36 (h, 6H, J=7.2 Hz), 1.18-1.07 (m, 6H), 0.91 (t, 9H, J=7.3 Hz).

### Step-4: Synthesis of 2′-methoxy-N,N,6-trimethyl-5′-nitro-[2,3′-bipyridin]-6′-amine

To a stirred solution of 5-bromo-6-methoxy-N,N-dimethyl-3-nitropyridin-2-amine (1.20 g, 4.346 mmol, 1.0 eq) and 2-methyl-6-(tributylstannyl)pyridine (2.50 g, 6.520 mmol, 1.5 eq) in toluene (20 mL) was added a LiCl (0.370 g, 8.692 mmol, 2.0 eq) at RT. The resulting mixture was purged with nitrogen for 10 min followed by addition of Pd(PPh₃)₄ (0.10 g, 0.087 mmol, 0.02 eq) and again purged with nitrogen for 10 min. The reaction mixture was heated at 150° C. for overnight. The progress of reaction was monitored by LCMS. The reaction mixture was diluted with water (50 mL), extracted with EtOAc (2×100 mL). The combined organic layers were washed with water (50 mL), with brine (50 mL), dried over Na₂SO₄, concentrated and purified by combi flash chromatography [silica gel 100-200 mesh: elution 0-30% EtOAc in Hexane] to afford the desired compound 2′-methoxy-N,N,6-trimethyl-5′-nitro-[2,3′-bipyridin]-6′-amine (1.05 g, 84.0%) as yellow solid. LCMS: (M+1)⁺: 289.1

### Step-5: Synthesis of 6′-(dimethylamino)-6-methyl-5′-nitro-[2,3′-bipyridin]-2′(1′H)-one

To a stirred solution of 2′-methoxy-N,N,6-trimethyl-5′-nitro-[2,3′-bipyridin]-6′-amine (1.0 g, 3.468 mmol, 1.0 eq) in DMF (10 mL) was added 21% solution of CH₃SNa (0.973 g, 13.873 mmol, 4.0 eq) in water (4.6 mL) at RTrt. The reaction mixture was stirred at 80° C. for overnight. The progress of reaction was monitored by LCMS. The reaction mixture was acidified with 1N HCl (5.0 mL). The reaction mixture extracted with DCM (2×50 mL). The combined organic layers were washed with water (50 mL), with brine (50 mL), dried over Na₂SO₄, concentrated and purified by combi flash chromatography [silica gel 100-200 mesh: elution 0-5.0% MeOH in DCM] to afford the desired compound 6′-(dimethylamino)-6-methyl-5′-nitro-[2,3′-bipyridin]-2′(1′H)-one (0.780 g, 82.01%) as yellow solid. LCMS: (M+1)⁺: 275.2

### Step-6: Synthesis of 5′-amino-6′-(dimethylamino)-6-methyl-[2,3′-bipyridin]-2′(1′H)-one

To a stirred solution of 6′-(dimethylamino)-6-methyl-5′-nitro-[2,3′-bipyridin]-2′(1¹H)-one (0.780 g, 2.843 mmol, 1.0 eq) in EtOH (25 mL) was added Fe (1.25 g, 22.750 mmol, 8.0 eq) and a solution of NH₄Cl (1.52 g, 28.43 mmol, 10.0 eq) in water (25 mL) at RT. The resulting mixture was heated at 90° C. for 60 min. The progress of reaction was monitored by LCMS. The reaction mixture was filtered through celite the residue was washed with EtOH (50 mL) the filtrate was concentrated and the residue was dissolved in EtOAc (200 mL), washed with water (2×100 mL), dried over Na₂SO₄, and concentrated to afford the desired 5′-amino-6′-(dimethylamino)-6-methyl-[2,3′-bipyridin]-2′(1′H)-one (1.145 g, 90.38%) as brown solid. LCMS: (M+1)⁺: 245.2

### Step-7: Synthesis of tert-butyl 4-((6′-(dimethylamino)-6-methyl-2′-oxo-1′,2′-dihydro-[2,3′-bipyridin]-5′-yl)amino)-1H-pyrrolo[2,3-b]pyridine-1-carboxylate

To a stirred solution of compound 5′-amino-6′-(dimethylamino)-6-methyl-[2,3′-bipyridin]-2′(1′H)-one (0.30 g, 1.227 mmol, 1.0 eq) and tert-butyl 4-chloro-1H-pyrrolo[2,3-b]pyridine-1-carboxylate (0.341 g, 1.350 mmol, 1.0 eq) in dioxane (20 mL) was added Cs₂CO₃ (1.60 g, 4.908 mmol, 4.0 eq) at RT. The resulting mixture was purged with nitrogen for 10 min followed by addition of Pd₂(dba)₃ (0.135 g, 0.147 mmol, 0.12 eq) and xantphos (0.106 g, 0.184 mmol, 0.15 eq) and again purged with nitrogen for 10 min. The reaction mixture was heated at 100° C. for overnight. The progress of reaction was monitored by LCMS. The reaction mixture was diluted with water (50 mL), extracted with EtOAc (2×100 mL). The combined organic layers were washed with water (50 mL), with brine (50 mL), dried over Na₂SO₄, concentrated and purified by combi flash chromatography [silica gel 100-200 mesh: elution 0-60% EtOAc in Hexane] to afford the desired compound tert-butyl 4-((6′-(dimethylamino)-6-methyl-2′-oxo-1′,2′-dihydro-[2,3′-bipyridin]-5′-yl)amino)-1H-pyrrolo [2,3-b]pyridine-1-carboxylate (40 mg, 7.07%%) as brown solid. LCMS: (M+1)⁺:−461.3

### Step-8: Synthesis of 5′-((1H-pyrrolo[2,3-b]pyridin-4-yl)amino)-6′-(dimethylamino)-6-methyl-[2,3′-bipyridin]-2′(1′H)-one

tert-butyl 4-((6′-(dimethylamino)-6-methyl-2′-oxo-1′,2′-dihydro-[2,3′-bipyridin]-5′-yl)amino)-1H-pyrrolo [2,3-b]pyridine-1-carboxylate (40 mg, 0.087 mmol, 1.0 eq) was dissolved in 4.0 M-HCl in dioxane (1.0 mL) and allowed to stir at RT for 1 h. After completion of reaction, the reaction mixture was filtered and dried under reduced pressure and purified by reverse phase purification to afford the desired compound 5′-((1H-pyrrolo[2,3-b]pyridin-4-yl)amino)-6′-(dimethylamino)-6-methyl-[2,3′-bipyridin]-2′(1′H)-one (4.0 mg, 12.90%) as light yellow solid. LCMS: (M+1)⁺: 361.3, UPLC: At 254 nm=95.71%, At 220 nm=96.81%.

¹H NMR (400 MHz, DMSO-d₆): δ 11.19 (brs, 1H), 8.21 (brs, 1H), 8.09 (s, 1H), 8.05 (s, 1H), 7.83 (brs, 1H), 7.71-7.79 (m, 2H), 7.07-7.17 (m, 2H), 6.47 (brs, 1H), 5.88 (d, J=5.70 Hz, 1H), 3.15 (s, 3H), 2.98 (s, 6H).

### Example-14: Synthesis of 3-(5-chloro-2-fluorophenyl)-6-(dimethylamino)-5-(pyridin-4-ylamino)pyridin-2(1H)-one: (Compound 1.14)

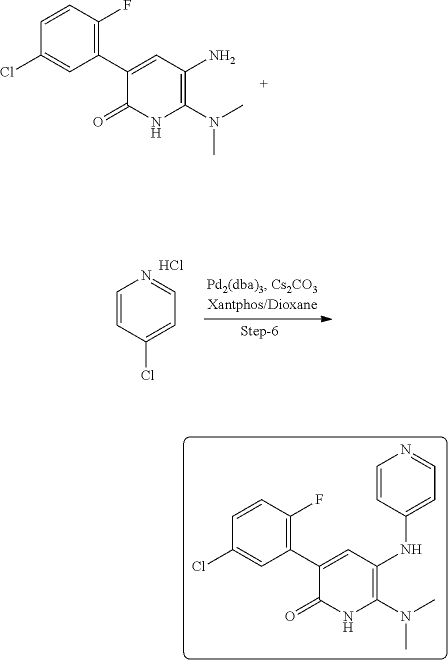 [SMILES: CN(C)c1[nH]c(=O)c(-c2cc(Cl)ccc2F)cc1N ; Cl ; Clc1ccncc1 ; CN(C)c1[nH]c(=O)c(-c2cc(Cl)ccc2F)cc1Nc1ccncc1]

Steps 1 to 5 are the same as in Example 9.

### Step-6: Synthesis of 3-(5-chloro-2-fluorophenyl)-6-(dimethylamino)-5-(pyridin-4-ylamino)pyridin-2(1H)-one

To a stirred solution of compound 5-amino-3-(5-chloro-2-fluorophenyl)-6-(dimethylamino)pyridin-2(1H)-one (300 mg, 1.065 mmol, 1.0 eq) and 4-chloropyridine hydrochloride (176 mg, 1.171 mmol, 1.1 eq) in dioxane (20 mL) was added Cs₂CO₃ (1.39 g, 4.26 mmol, 4.0 eq) at RT. The resulting mixture was purged with nitrogen for 10 min followed by addition of Pd₂(dba)₃ (117 mg, 0.127 mmol, 0.12 eq) and xantphos (92 mg, 0.159 mmol, 0.15 eq), again purged with nitrogen for 10 min. The reaction mixture was heated at 100° C. for overnight. The progress of reaction was monitored by LCMS. The reaction mixture was diluted with water (50 mL), extracted with EtOAc (2×100 mL). The combined organic layers were washed with water (50 mL), with brine (50 mL), dried over Na₂SO₄, concentrated and purified by combi flash chromatography [silica gel 100-200 mesh: elution 0-10% MeOH in DCM] to afford the desired compound 3-(5-chloro-2-fluorophenyl)-6-(dimethylamino)-5-(pyridin-4-ylamino)pyridin-2(1H)-one (140 mg, 36.55%) as brown solid. LCMS: (M+1)⁺359.2, UPLC: At 254 nm: 93.58%, At 220 nm: 91.38%.

¹H NMR (400 MHz, DMSO-d₆) δ 10.75 (brs, 1H), 8.20 (brs, 1H), 8.07 (d, J=6.14 Hz, 2H), 7.54 (dd, J=2.63, 6.58 Hz, 1H), 7.35 (dd, J=4.39, 7.45 Hz, 1H), 7.22-7.33 (m, 2H), 6.50 (d, J=5.70 Hz, 2H), 2.90 (s, 6H).

### Example-15: Synthesis of 3-(5-chloro-2-fluorophenyl)-6-(dimethylamino)-5-((3-(trifluoromethyl)pyridin-4-yl)amino)pyridin-2(1H)-one: (Compound 1.15)

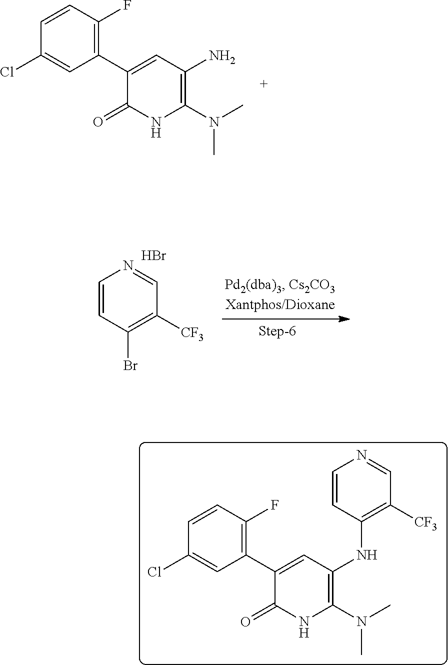 [SMILES: CN(C)c1[nH]c(=O)c(-c2cc(Cl)ccc2F)cc1N ; Br ; FC(F)(F)c1cnccc1Br ; CN(C)c1[nH]c(=O)c(-c2cc(Cl)ccc2F)cc1Nc1ccncc1C(F)(F)F]

Steps 1 to 5 are the same as in Example 9.

### Step-6: Synthesis of 3-(5-chloro-2-fluorophenyl)-6-(dimethylamino)-5-((3-(trifluoromethyl)pyridin-4-yl)amino)pyridin-2(1H)-one

To a stirred solution of compound 5-amino-3-(5-chloro-2-fluorophenyl)-6-(dimethylamino)pyridin-2(1H)-one (200 mg, 0.709 mmol, 1.0 eq) and 4-bromo-3-(trifluoromethyl)pyridine hydrobromide (240 mg, 0.781 mmol, 1.1 eq) in dioxane (20 mL) was added Cs₂CO₃ (925 mg, 2.836 mmol, 4.0 eq) at rt. The resulting mixture was purged with nitrogen for 10 min followed by addition of Pd₂(dba)₃ (78 mg, 0.086 mmol, 0.12 eq) and xantphos (62 mg, 0.106 mmol, 0.15 eq), again purged with nitrogen for 10 min. The reaction mixture was heated at 100° C. for overnight. The progress of reaction was monitored by LCMS. The reaction mixture was diluted with water (50 mL), extracted with EtOAc (2×100 mL). The combined organic layers were washed with water (50 mL), with brine (50 mL), dried over Na₂SO₄, concentrated and purified by combi flash chromatography [silica gel 100-200 mesh: elution 0-5% MeOH in DCM] to afford the desired compound 3-(5-chloro-2-fluorophenyl)-6-(dimethylamino)-5-((3-(trifluoromethyl)pyridin-4-yl)amino)pyridin-2(1H)-one (80 mg, 26.40%) as brown solid. LCMS: (M+1)⁺427.2, UPLC:—At 254 nm: 98.45%, At 220 nm: 98.12%.

¹H NMR (400 MHz, DMSO-d₆): δ 10.86 (brs, 1H), 8.44 (s, 1H), 8.23 (d, J=6.14 Hz, 1H), 8.01 (brs, 1H), 7.54 (brs, 1H), 7.31-7.40 (m, 1H), 7.20-7.31 (m, 2H), 6.31 (d, J=5.26 Hz, 1H), 2.92 (s, 6H).

### Example-16: Synthesis of 3-(5-chloro-2-fluorophenyl)-6-(dimethylamino)-5-(quinolin-4-ylamino)pyridin-2(1H)-one: (Compound 1.16)

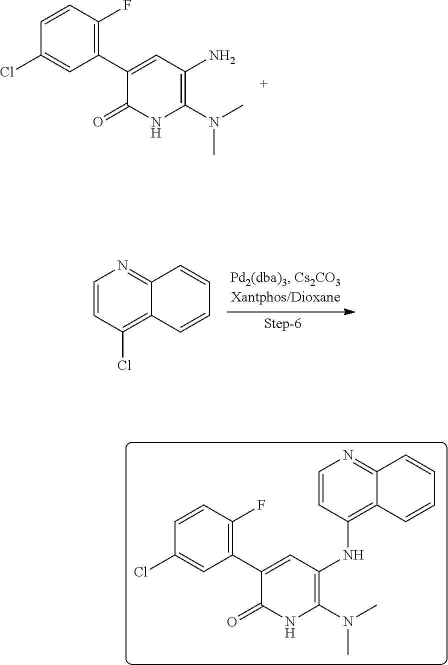 [SMILES: CN(C)c1[nH]c(=O)c(-c2cc(Cl)ccc2F)cc1N ; Clc1ccnc2ccccc12 ; CN(C)c1[nH]c(=O)c(-c2cc(Cl)ccc2F)cc1Nc1ccnc2ccccc12]

Steps 1 to 5 are the same as in Example 9.

### Step-6: Synthesis of 3-(5-chloro-2-fluorophenyl)-6-(dimethylamino)-5-(quinolin-4-ylamino)pyridin-2(1H)-one

To a stirred solution of compound 5-amino-3-(5-chloro-2-fluorophenyl)-6-(dimethylamino)pyridin-2(1H)-one (200 mg, 0.709 mmol, 1.0 eq) and 4-chloroquinoline (128 mg, 0.781 mmol, 1.1 eq) in dioxane (20 mL) was added Cs₂CO₃ (925 mg, 2.836 mmol, 4.0 eq) at rt. The resulting mixture was purged with nitrogen for 10 min followed by addition of Pd₂(dba)₃ (78 mg, 0.086 mmol, 0.12 eq) and xantphos (62 mg, 0.106 mmol, 0.15 eq), again purged with nitrogen for 10 min. The reaction mixture was heated at 100° C. for overnight. The progress of reaction was monitored by LCMS. The reaction mixture was diluted with water (50 mL), extracted with EtOAc (2×100 mL). The combined organic layers were washed with water (50 mL), with brine (50 mL), dried over Na₂SO₄, concentrated and purified by reverse phase chromatography to afford the desired compound 3-(5-chloro-2-fluorophenyl)-6-(dimethylamino)-5-(quinolin-4-ylamino)pyridin-2(1H)-one (35 mg, 12.02%) as yellow solid. LCMS: (M+1)⁺409.3, UPLC: At 254 nm: 96.95%, At 220 nm: 98.14%.

¹H NMR (400 MHz, DMSO-d₆): δ 14.15 (brs., 1H), 11.06 (brs, 1H), 10.35 (brs, 1H), 8.67 (d, J=8.77 Hz, 1H), 8.50 (d, J=6.58 Hz, 1H), 8.13 (s, 1H), 7.99 (brs, 2H), 7.76 (brs, 1H), 7.53 (s, 2H), 7.39 (d, J=8.33 Hz, 1H), 7.28 (t, J=9.20 Hz, 1H), 6.44 (d, J=5.72 Hz, 1H), 2.93 (s, 6H).

### Example-17: Synthesis of (S)-4-((5-(5-chloro-2-fluorophenyl)-2-(dimethylamino)-1-methyl-6-oxo-1, 6-dihydropyridin-3-yl)amino)-N-(1-hydroxypropan-2-yl)nicotinamide: (Compound 1.17)

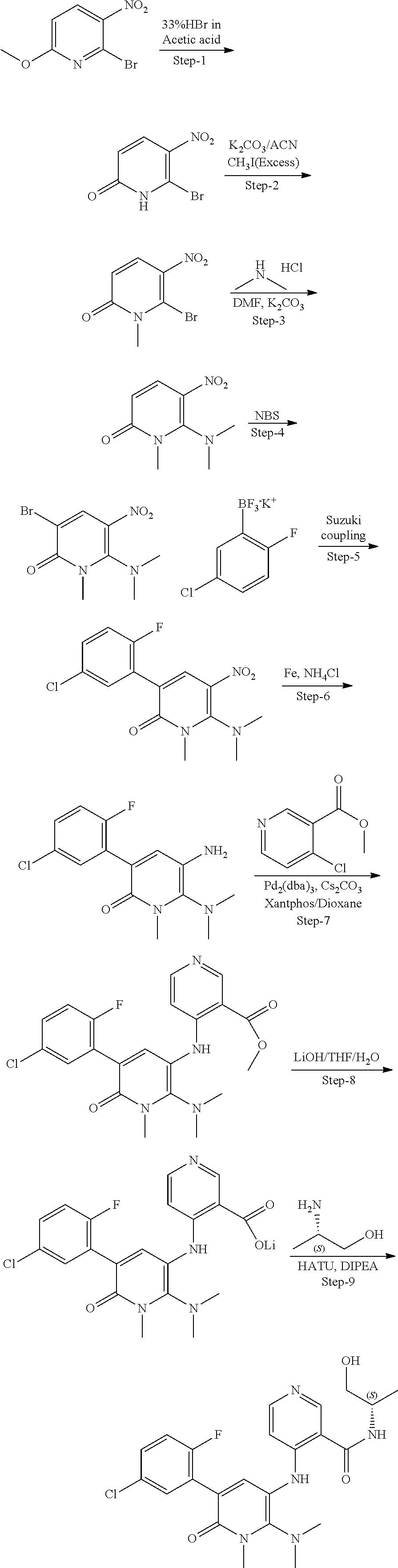

### Step-1: Synthesis of 6-bromo-5-nitropyridin-2(1H)-one

2-bromo-6-methoxy-3-nitropyridine (8.0 g, 34.632 mmol, 1.0 eq) was dissolved in 33% HBr in acetic acid (100 mL) was added at RT. The reaction mixture was stirred at 80° C. for overnight. The progress of reaction was monitored by LCMS. The formation of precipitates was observed, upon the addition of diethyl ether (200 mL), filtered and washed with diethyl ether (50 mL), dried under vacuum to afford the desired compound 6-bromo-5-nitropyridin-2(1H)-one (5.50 g, 94.37%) as brown solid. LCMS: (M+1)⁺: 218.9

### Step-2: Synthesis of 6-bromo-1-methyl-5-nitropyridin-2(1H)-one

To a stirred solution of 6-bromo-5-nitropyridin-2(1H)-one (3.0 g, 13.70 mmol, 1.0 eq) and K₂CO₃ (7.57 g, 41.097 mmol, 4.0 eq) in acetonitrile (20 mL) was added methyl iodide (2.6 mL, 54.8 mmol, 3.0 eq) at rt. The reaction mixture was stirred at 60° C. for overnight. The progress of reaction was monitored by LCMS. The reaction mixture was poured in ice cold water (50 mL), extracted with EtOAc (2×100 mL). The combined organic layers were washed with water (50 mL), with brine (50 mL), dried over Na₂SO₄, concentrated and purified by combi flash chromatography [silica gel 100-200 mesh: elution 0-30% EtOAc in Hexane], to afford the desired compound 6-bromo-1-methyl-5-nitropyridin-2(1H)-one (0.55 g, 17.23%) as yellow solid.

¹H NMR (400 MHz, CDCl₃): δ 7.95 (d, J=10.09 Hz, 1H), 6.50 (d, J=9.65 Hz, 1H), 4.00 (s, 3H).

### Step-3: Synthesis of 6-(dimethylamino)-1-methyl-5-nitropyridin-2(1H)-one

To a stirred solution of 6-bromo-1-methyl-5-nitropyridin-2(1H)-one (0.65 g, 2.79 mmol, 1.0 eq) and dimethylamine hydrochloride (0.273 g, 3.347 mmol, 1.2 eq) in DMF (20 mL) was added K₂CO₃ (1.16 g, 8.37 mmol, 3.0 eq) at rt. The reaction mixture was stirred at 90° C. for 3 h. The progress of reaction was monitored by LCMS. The reaction mixture was poured into ice cold water (50 mL), extracted with EtOAc (2×100 mL). The combined organic layers were washed with water (50 mL), with brine (50 mL), dried over Na₂SO₄, and concentrated to afford the desired compound 6-(dimethylamino)-1-methyl-5-nitropyridin-2(1H)-one (0.23 g, 41.81%) as yellow solid. LCMS: (M+1)⁺: 198.1

### Step-4: Synthesis of 3-bromo-6-(dimethylamino)-1-methyl-5-nitropyridin-2(1H)-one

To a stirred solution of 6-(dimethylamino)-1-methyl-5-nitropyridin-2(1H)-one (0.23 g, 1.166 mmol, 1.0 eq) in DMF (50 mL) was added NBS (0.228 g, 1.283 mmol, 1.1 eq) at rt. The reaction mixture was stirred at rt for 1 h. The progress of reaction was monitored by LCMS. The reaction mixture was poured into ice cold water (50 mL), extracted with EtOAc (2×100 mL). The combined organic layers were washed with water (50 mL), with brine (50 mL), dried over Na₂SO₄, and concentrated to afford the desired compound 3-bromo-6-(dimethylamino)-1-methyl-5-nitropyridin-2(1H)-one (300 mg, 93.16%) as yellow solid. LCMS: (M+1)⁺: 276.1

### Step-5: Synthesis of 3-(5-chloro-2-fluorophenyl)-6-(dimethylamino)-1-methyl-5-nitropyridin-2(1H)-one

To a stirred solution of 3-bromo-6-(dimethylamino)-1-methyl-5-nitropyridin-2(1H)-one (300 mg, 1.09 mmol, 1.0 eq) and potassium (5-chloro-2-fluorophenyl)trifluoroborate (308 mg, 1.30 mmol, 1.2 eq) in dioxane (10 mL) was added a 2M solution of Na₂CO₃ (231 mg, 2.18 mmol, 2.0 eq) in H₂O (1.09 mL) at rt. The resulting mixture was purged with nitrogen for 10 min followed by addition of Pd(dppf)Cl₂.DCM (45 g, 0.054 mmol, 0.05 eq), again purged with nitrogen for 10 min. The reaction mixture was heated at 100° C. for overnight. The progress of reaction was monitored by LCMS. The reaction mixture was diluted with water (50 mL), extracted with EtOAc (2×100 mL). The combined organic layers were washed with water (50 mL), with brine (50 mL), dried over Na₂SO₄, concentrated and purified by combi flash chromatography [silica gel 100-200 mesh: elution 0-30% EtOAc in Hexane] to afford the desired 3-(5-chloro-2-fluorophenyl)-6-(dimethylamino)-1-methyl-5-nitropyridin-2(1H)-one (220 mg, 62.32%) as yellow solid. LCMS: (M+1)⁺: 326.2

### Step-6: Synthesis of 5-amino-3-(5-chloro-2-fluorophenyl)-6-(dimethylamino)-1-methylpyridin-2(1H)-one

To a stirred solution of 3-(5-chloro-2-fluorophenyl)-6-(dimethylamino)-1-methyl-5-nitropyridin-2(1H)-one (220 mg, 0.654 mmol, 1.0 eq) in EtOH (10 mL) was added Fe (302 mg, 5.403 mmol, 8.0 eq) and a solution of NH₄Cl (350 mg, 6.54 5 mmol, 10.0 eq) in H₂O (10 mL) at rt. The resulting mixture was heated at 90° C. for 60 min. The progress of reaction was monitored by LCMS. The reaction mixture was filtered through celite the residue was washed with EtOH (50 mL) the filtrate was concentrated and the residue was dissolved in EtOAc (200 mL), washed with water (2×100 mL), dried over Na₂SO₄, and concentrated to afford the desired 5-amino-3-(5-chloro-2-fluorophenyl)-6-(dimethylamino)-1-methylpyridin-2(1H)-one (190 mg, 95.47%) as green viscous semisolid. LCMS: (M+1)⁺: 296.2

### Step-7: Synthesis of methyl 4-((5-(5-chloro-2-fluorophenyl)-2-(dimethylamino)-1-methyl-6-oxo-1,6-dihydropyridin-3-yl)amino)nicotinate

To a stirred solution of compound 5-amino-3-(5-chloro-2-fluorophenyl)-6-(dimethylamino)-1-methylpyridin-2(1H)-one (190 mg, 0.642 mmol, 1.0 eq) and methyl 4-chloronicotinate (122 mg, 0.706 mmol, 1.0 eq) in dioxane (10 mL) was added Cs₂CO₃ (837 mg, 2.568 mmol, 4.0 eq) at rt. The resulting mixture was purged with nitrogen for 10 min followed by addition of Pd₂(dba)₃ (70 mg, 0.077 mmol, 0.12 eq) and xantphos (55 mg, 0.096 mmol, 0.15 eq), again purged with nitrogen for 10 min. The reaction mixture was heated at 100° C. for overnight. The progress of reaction was monitored by LCMS. The reaction mixture was diluted with water (50 mL), extracted with EtOAc (2×100 mL). The combined organic layers were washed with water (50 mL), with brine (50 mL), dried over Na₂SO₄, concentrated and purified by combi flash chromatography [silica gel 100-200 mesh: elution 0-10% MeOH in DCM] to afford the desired compound methyl 4-((5-(5-chloro-2-fluorophenyl)-2-(dimethylamino)-1-methyl-6-oxo-1,6-dihydropyridin-3-yl)amino)nicotinate (60 mg, 21.66%) as brown solid. LCMS: (M+1)⁺: 431.2

### Step-8: Synthesis of lithium 4-((5-(5-chloro-2-fluorophenyl)-2-(dimethylamino)-1-methyl-6-oxo-1,6-dihydropyridin-3-yl)amino)nicotinate

To a stirred solution of methyl 4-((5-(5-chloro-2-fluorophenyl)-2-(dimethylamino)-1-methyl-6-oxo-1,6-dihydropyridin-3-yl)amino)nicotinate (60 mg, 0.139 mmol, 1.0 eq) in THF (4.0 mL) was added a solution of LiOH.H₂O (12 mg, 0.278 mmol, 2.0 eq) in water (1.0 mL) at rt. The reaction mixture was stirred at for overnight. The progress of reaction was monitored by LCMS. The reaction mixture was concentrated to afford the desired lithium 4-((5-(5-chloro-2-fluorophenyl)-2-(dimethylamino)-1-methyl-6-oxo-1,6-dihydropyridin-3-yl)amino)nicotinate (58 mg, 98.52%) as brown solid. LCMS: (M+1)⁺: 417.2

### Step-9: Synthesis of (S)-4-((5-(5-chloro-2-fluorophenyl)-2-(dimethylamino)-1-methyl-6-oxo-1,6-dihydropyridin-3-yl)amino)-N-(1-hydroxypropan-2-yl)nicotinamide

To a stirred solution of lithium 4-((5-(5-chloro-2-fluorophenyl)-2-(dimethylamino)-1-methyl-6-oxo-1,6-dihydropyridin-3-yl)amino)nicotinate (60 mg, 0.142 mmol, 1.0 eq) in DMF (2 mL) was added HATU (81 mg, 0.213 mmol, 1.5 eq) at rt. The resulting mixture was stirred at rt for 5 minutes, followed by addition of (S)-2-aminopropan-1-ol (21 mg, 0.288 mmol, 2.0 eq) and DIPEA (0.074 mL, 0.426 mmol, 3.0 eq) and stirred at rt for 30 min. Reaction mixture was diluted with water (50 mL) and extracted with ethyl acetate (2×20 mL). The combined organic extracts were washed with water (50 mL), with brine (50 mL) dried over Na₂SO₄ and concentrated under reduced and purified by combi flash chromatography [silic gel 100-200 mesh; elution 0-10% MeOH in DCM] to afford the desired compound (S)-4-((5-(5-chloro-2-fluorophenyl)-2-(dimethylamino)-1-methyl-6-oxo-1,6-dihydropyridin-3-yl) amino)-N-(1-hydroxypropan-2-yl)nicotinamide (10 mg, 14.92%) as brown solid. LCMS: (M+1)⁺474.3, UPLC: At 254 nm: 96.89%, At 220 nm: 99.58%.

¹H NMR (400 MHz, DMSO-d₆): δ 9.81 (brs, 1H), 8.70 (s, 1H), 8.46 (brs, 1H), 8.18 (d, J=6.14 Hz, 1H), 7.58 (d, J=6.14 Hz, 1H), 7.43 (s, 2H), 7.22-7.32 (m, 1H), 6.63 (brs, 1H), 4.79 (brs, 1H), 4.05 (brs, 1H), 3.48 (s, 3H), 2.74 (brs, 6H), 2.08 (s, 3H), 1.51 (brs, 1H), 1.23 (brs, 1H), 1.15 (d, J=6.58 Hz, 3H).

### Example-18: Synthesis of 8-((1H-pyrrolo[2,3-b]pyridin-4-yl)amino)-6-(5-chloro-2-fluorophenyl)-1-methyl-2,3-dihydroimidazo[1,2-a]pyridin-5(1H)-one: (Compound 1.18)

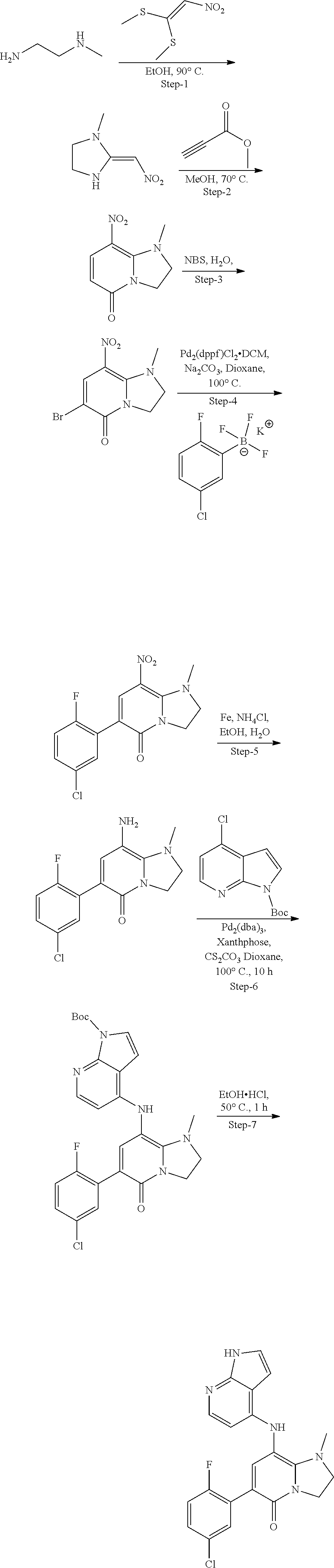

### Step-1: Synthesis of 1-methyl-2-(nitromethylene)imidazolidine

To a stirred solution of N¹-methylethane-1, 2-diamine (5 g, 3.03 mmol, 1.0 eq) in EtOH (30 mL), was added (2-nitroethene-1, 1-diyl)bis(methylsulfane) (2.24 g, 3.03 mmol, 1.0 eq), The reaction mixture was allowed to stir for 3 h at 90° C. Progress of the reaction was monitored by TLC and LCMS. After completion of the reaction, the reaction mixture was concentrated under reduced pressure to obtain crude, which was triturated from hexane to obtain 1-methyl-2-(nitromethylene)imidazolidine (4.0 g, 91%) as a yellow solid. LCMS: (M+1)⁺: 144.0

### Step-2: Synthesis of 1-methyl-8-nitro-2,3-dihydroimidazo[1,2-a]pyridin-5(1H)-one

To a stirred solution of 1-methyl-2-(nitromethylene)imidazolidine (2.0 g, 14.0 mmol, 1.0 eq) in MeOH (12 mL) methyl propiolate (1.28 g, 15.0 mmol, 1.1 eq). The resulting mixture was was heated at 70° C. for 3 h. The progress of reaction was monitored by LCMS. The reaction mixture was cooled to rt. Desired product was precipitated out was filtered & washed with MeOH (10 ml) to afford the desired compound 1-methyl-8-nitro-2,3-dihydroimidazo[1,2-a]pyridin-5(1H)-one (1.0 g, 37.03%) as yellow solid. LCMS: (M+1)⁺: 196.0

### Step-3: Synthesis of 6-bromo-1-methyl-8-nitro-2, 3-dihydroimidazo [1, 2-a]pyridin-5(1H)-one

To a stirred solution of compound 1-methyl-8-nitro-2,3-dihydroimidazo[1,2-a]pyridin-5(1H)-one (2.0 g, 10.30 mmol, 1.0 eq) in H₂O (20 mL) was added NBS (1.83 g, 10.03 mmol, 1 eq) at 0° C. The resulting mixture was stirred at 0° C. to rt for 4 h. The progress of reaction was monitored by LCMS. On Completion, desired product was precipitated out which was separated by filtration and washed with H₂O (10 ml) to afford the desired compound 6-bromo-1-methyl-8-nitro-2,3-dihydroimidazo[1,2-a]pyridin-5(1H)-one (2.0 g, 71.94%) as yellow solid. LCMS: (M+1)⁺: 274.0

### Step-4: Synthesis of 6-(5-chloro-2-fluorophenyl)-1-methyl-8-nitro-2,3-dihydroimidazo[1,2-a]pyridin-5(1H)-one

To a stirred solution of 6-bromo-1-methyl-8-nitro-2,3-dihydroimidazo[1,2-a]pyridin-5(1H)-one (0.5 g, 1.831 mmol, 1.0 eq) & potassium (5-chloro-2-fluorophenyl)trifluoroborate (0.95 g, 4.029 mmol, 2.2 eq) and 2M Na₂CO₃ (2 ml, 3.663 mmol, 2 eq) in dioxane (10 mL). The resulting mixture was purged with nitrogen for 10 min followed by addition of Pd₂(dppf)Cl₂.DCM complex (0.134 g, 0.183 mmol, 0.12 eq) again purged with nitrogen for 10 min. The reaction mixture was heated at 100° C. for overnight. The progress of reaction was monitored by LCMS. The reaction mixture was diluted with water (30 mL), extracted with EtOAc (2×50 mL). The combined organic layers were washed with water (30 mL), with brine (30 mL), dried over Na₂SO₄, concentrated to afford crude, which was purified by column chromatography to afford the desired 6-(5-chloro-2-fluorophenyl)-1-methyl-8-nitro-2,3-dihydroimidazo[1,2-a]pyridin-5(1H)-one (600 mg, 80.05%) as yellowish solid. LCMS: (M+1)⁺: 324.1

### Step-5: Synthesis of 8-amino-6-(5-chloro-2-fluorophenyl)-1-methyl-2,3-dihydroimidazo[1,2-a]pyridin-5(1H)-one

To a stirred solution of 6-(5-chloro-2-fluorophenyl)-1-methyl-8-nitro-2,3-dihydroimidazo[1,2-a]pyridin-5(1H)-one (0.60 g, 1.857 mmol, 1.0 eq) in 60 mL EtOH: H₂O (1:1 ratio) was added Fe (0.728 g, 13.0 mmol, 7.0 eq) followed by NH₄Cl (0.702 g, 13.0 mmol, 7.0 eq) at room temperature. The resulting mixture was stirred at 90° C. for 3 h. The progress of reaction was monitored by LCMS. The reaction mixture was filtered through celite the filtrate was concentrated, diluted with water (30 mL), extracted with EtOAc (2×50 mL). The combined organic layers were washed with brine (50 mL), dried over Na₂SO₄, concentrated to afford the desired compound 8-amino-6-(5-chloro-2-fluorophenyl)-1-methyl-2,3-dihydroimidazo[1,2-a]pyridin-5(1H)-one (0.40 g, 76.33%) as green solid. LCMS: (M+1)⁺: 294.1

### Step-6: Synthesis of tert-butyl 4-((6-(5-chloro-2-fluorophenyl)-1-methyl-5-oxo-1,2,3,5-tetrahydroimidazo[1,2-a]pyridin-8-yl)amino)-1H-pyrrolo[2,3-b]pyridine-1-carboxylate

To a stirred solution of 8-amino-6-(5-chloro-2-fluorophenyl)-1-methyl-2,3-dihydroimidazo[1,2-a]pyridin-5(1H)-one (40 mg, 0.14 mmol, 1 equiv), tert-butyl 4-chloro-1H-pyrrolo[2,3-b]pyridine-1-carboxylate (36 mg, 0.149 mmol, 1.1 equiv) and CS₂CO₃(91 mg, 0.28 mmol, 2 equiv) in dioxane (3 mL). The resulting mixture was degassed with nitrogen for 10 min followed by addition of Pd₂(dba)₃ (13 mg, 0.014 mmol, 0.1 equiv) and xantphos (0.16, 0.028 mmol, 0.2 equiv), again purged with nitrogen for 10 min. The reaction mixture was stirred at 100° C. for overnight. The progress of reaction was monitored by LCMS. The reaction mixture was diluted with water (30 mL), extracted with EtOAc (2×50 mL). The combined organic layers were washed with water (30 mL), with brine (30 mL), dried over Na₂SO₄, concentrated under vacuum to afford 50 mg of crude product used directly for next step. LCMS: (M+1)⁺: 509.16

### Step-7: Synthesis of 8-((1H-pyrrolo[2,3-b]pyridin-4-yl)amino)-6-(5-chloro-2-fluorophenyl)-1-methyl-2,3-dihydroimidazo[1,2-a]pyridin-5(1H)-one

A solution of tert-butyl 4-((6-(5-chloro-2-fluorophenyl)-1-methyl-5-oxo-1,2,3,5-tetrahydroimidazo[1,2-a]pyridin-8-yl)amino)-1H-pyrrolo[2,3-b]pyridine-1-carboxylate (50 mg, 0.1 mmol, 1.0 eq) in ethanolic HCl (1.25 M) was stirred at 50° C. for 1 h. The progress of reaction was monitored by LCMS. The reaction mixture was concentrated under vacuum. Crude was purified by reverse phase HPLC to afford 20 mg of 8-((1H-pyrrolo[2,3-b]pyridin-4-yl)amino)-6-(5-chloro-2-fluorophenyl)-1-methyl-2,3-dihydroimidazo[1,2-a]pyridin-5(1H)-one. LCMS: (M+1)⁺: 409.11

¹H NMR (400 MHz, DMSO-d₆): δ 11.24 (s, 1H), 8.20 (s, 1H), 7.86 (s, 1H), 7.82 (d, J=5.5 Hz, 1H), 7.66 (dd, J=6.6, 2.8 Hz, 1H), 7.33 (s, 1H), 7.29 (dt, J=7.1, 3.5 Hz, 1H), 7.21 (t, J=9.6 Hz, 1H), 7.15-7.08 (m, 1H), 6.54 (d, J=8.5 Hz, 1H), 5.99 (d, J=5.5 Hz, 1H), 4.09 (t, J=8.9 Hz, 2H), 3.66 (t, J=8.9 Hz, 2H), 2.90 (s, 3H).

### Example-19: Synthesis of (S)-4-((6-(5-chloro-2-fluorophenyl)-1-methyl-5-oxo-1,2,3,5-tetrahydroimidazo[1,2-a]pyridin-8-yl)amino)-N-(2-hydroxypropyl)nicotinamide: (Compound 1.19)

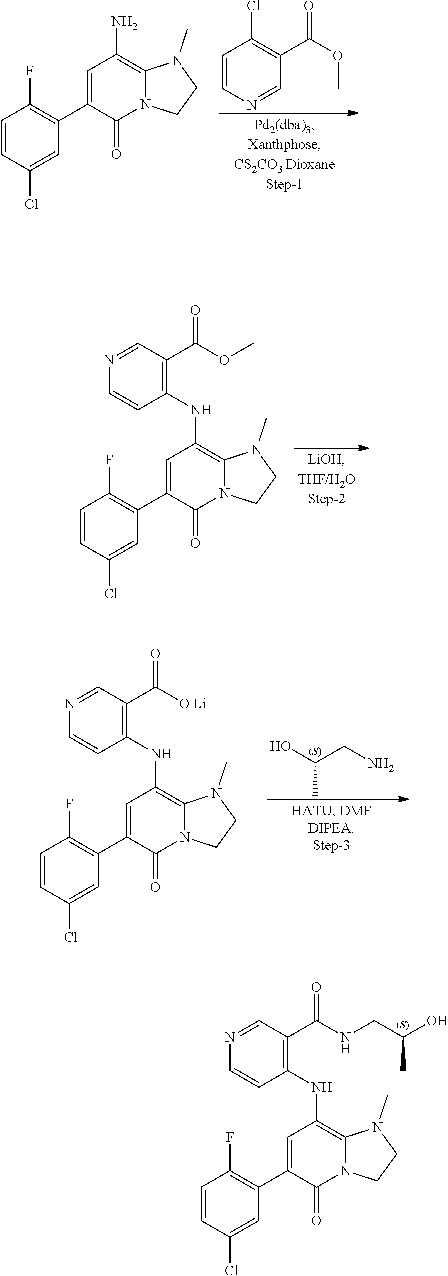

### Step-1: Synthesis of methyl 4-((6-(5-chloro-2-fluorophenyl)-1-methyl-5-oxo-1,2,3,5-tetrahydroimidazo [1,2-a]pyridin-8-yl)amino)nicotinate

To a stirred solution of 8-amino-6-(5-chloro-2-fluorophenyl)-1-methyl-2,3-dihydroimidazo[1,2-a]pyridin-5(1H)-one (0.35 g, 1.194 mmol, 1.0 eq) & methyl 4-chloronicotinate (0.25 g, 1.314 mmol, 1.1 eq) and CS₂CO₃(0.776 g, 2.389 mmol, 2.0 eq) in dioxane (10.0 mL). The resulting mixture was purged with nitrogen for 10 min followed by addition of Pd₂(dba)₃ (0.110 g, 0.119 mmol, 0.12 eq) and xantphos (0.138 g, 0.239 mmol, 0.2 eq), again purged with nitrogen for 10 min. The reaction mixture was heated at 100° C. for overnight. The progress of reaction was monitored by LCMS. The reaction mixture was diluted with water (30 mL), extracted with EtOAc (2×50 mL). The combined organic layers were washed with water (30 mL), with brine (30 mL), dried over Na₂SO₄, concentrated to afford the desired methyl 4-((6-(5-chloro-2-fluorophenyl)-1-methyl-5-oxo-1,2,3,5-tetrahydroimidazo[1,2-a]pyridin-8-yl)amino)nicotinate, (220 mg, 41.43%) as brown solid. LCMS: (M+1)⁺: 429.1

### Step-2: Synthesis of lithium 4-((6-(5-chloro-2-fluorophenyl)-1-methyl-5-oxo-1,2,3,5-tetrahydroimidazo[1,2-a]pyridin-8-yl)amino)nicotinate

To a stirred solution of methyl 4-((6-(5-chloro-2-fluorophenyl)-1-methyl-5-oxo-1,2,3,5-tetrahydroimidazo[1,2-a]pyridin-8-yl)amino)nicotinate (0.22 g, 0.513 mmol, 1.0 eq) in THF (6 mL). Was added a solution of LiOH.H₂O (0.03 g, 1.025 mmol, 2.0 eq) in water (2.0 mL) at rt. The reaction mixture was stirred at for 2 h. The progress of reaction was monitored by LCMS. The reaction mixture was concentrated to & triturate with Hexane afford the desired lithium 4-((6-(5-chloro-2-fluorophenyl)-1-methyl-5-oxo-1,2,3,5-tetrahydroimidazo[1,2-a]pyridin-8-yl)amino)nicotinate (200 mg, 94.33%) as yellow solid. LCMS: (M+1)⁺414.1

### Step-3: Synthesis of (S)-4-((6-(5-chloro-2-fluorophenyl)-1-methyl-5-oxo-1,2,3,5-tetrahydroimidazo[,1,2-a]pyridin-8-yl)amino)-N-(2-hydroxypropyl)nicotinamide

To a stirred solution of lithium 4-((6-(5-chloro-2-fluorophenyl)-1-methyl-5-oxo-1,2,3,5-tetrahydroimidazo[1,2-a]pyridin-8-yl)amino)nicotinate (0.050 g, 0.1207 mmol, 1.0 eq) in DMF (2 mL) was added HATU (0.069 g, 0.1810 mmol, 1.5 eq) at rt. The resulting mixture was stirred at rt for 5 minutes, followed by addition of (S)-1-aminopropan-2-ol (0.05 ml, 0.2415 mmol, 1.5 eq) and DIPEA (0.1 mL, 0.3621 mmol, 3.0 eq) and stirred at rt for 1 h. Reaction mixture was diluted with water (20 mL) and extracted with ethyl acetate (2×50 mL) The combined organic extracts were washed with water (50 mL), with brine (50 mL) dried over Na₂SO₄ and concentrated under reduced pressure and purified by prep purification to afford the desired compound (S)-4-((6-(5 eq-chloro-2-fluorophenyl)-1-methyl-5-oxo-1,2,3,5-tetrahydroimidazo[1,2-a]pyridin-8-yl)amino)-N-(2-hydroxypropyl)nicotinamide (10 mg, 12.0%) as off white solid. LCMS: (M+1)⁺472.3. UPLC: At 254 nm: 97.57%, At 220 nm: 96.85%.

¹H NMR (400 MHz, METHANOL-d₄): δ 9.49(1, 1H), 8.58 (s, 1H), 8.16 (d, J=6.1 Hz, 2H), 7.56 (dd, J=2.4, 6.8 Hz, 2H), 7.34 (s, 1H), 7.09 (d, J=9.6 Hz, 1H), 6.64 (d, J=6.1 Hz, 1H), 5.37 (d, 1H) 4.20 (d, J=9.2 Hz, 2H), 3.78 (m, 1H),3.76-3.66 (m, 2H), 3.51-3.41 (m, 2H), 3.02 (s, 3H), 1.24-1.14 (m, 3H).

### Example-20: Synthesis of (S)-4-((7-(5-chloro-2-fluorophenyl)-1-methyl-6-oxo-1,3,4,6-tetrahydro-2H-pyrido[1, 2-a]pyrimidin-9-yl)amino)-N-(2-hydroxypropyl)nicotinamide: (Compound 1.20)

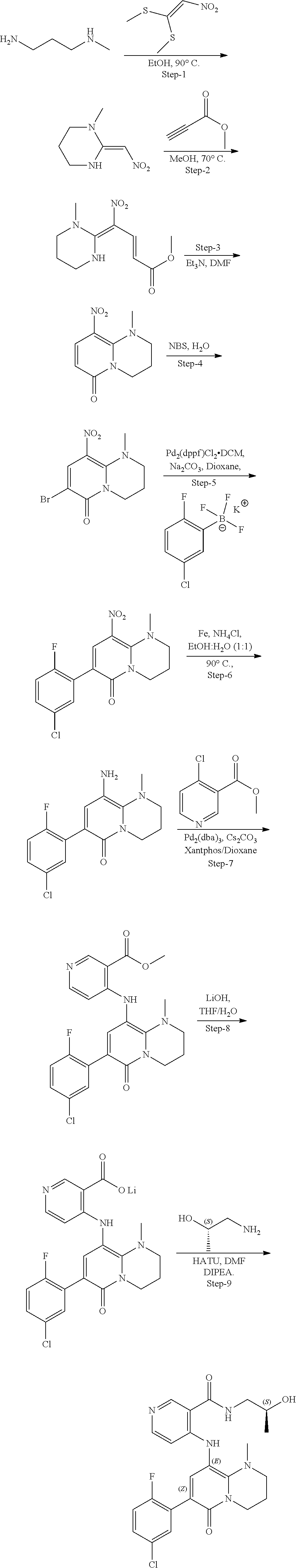

### Step-1: Synthesis of 1-methyl-2-(nitromethylene)hexhydropyrimidine

To a stirred solution of N¹-methylpropane-1, 3-diamine (2.5 g, 15.15 mmol, 1.0 eq) in EtOH (30 mL), was added (2-nitroethene-1, 1-diyl)bis(methylsulfane) (1.61 g, 15.15 mmol, 1.0 eq), The reaction mixture was allowed to stir for 4 h at 90° C. Progress of the reaction was monitored by TLC and LCMS. After completion of the reaction, the reaction mixture was concentrated under reduced pressure to obtain crude, which was triturated from hexane to obtain 1-methyl-2-(nitromethylene)hexahydropyrimidine (2.0 g, 83.68%) as a yellow solid. LCMS: (M+1)⁺: 158.1

### Step-2: Synthesis of methyl (2E,4E)-4-(1-methyltetrahydropyrimidin-2(1H)-ylidene)pent-2-enoate

To a stirred solution of 1-methyl-2-(nitromethylene)hexahydropyrimidine (3.0 g, 18.98 mmol, 1.0 eq) in MeOH (25 mL) methyl propiolate (1.8 g, 15.0 mmol, 1.1 eq). The resulting mixture was heated at 70 C for 3 h. The progress of reaction was monitored by LCMS. The reaction mixture was cooled to rt and the solution was concentrated to obtain crude product, which was washed with MeoH (10 ml) to afford the desired compound methyl (2E,4E)-4-(1-methyltetrahydropyrimidin-2(1H)-ylidene)pent-2-enoate (3.0 g, 65.63%) as brown solid. LCMS: (M+1)⁺: 242.1

### Step-3: Synthesis of 1-methyl-9-nitro-1,2,3,4-tetrahydro-6H-pyrido[1,2-a]pyrimidin-6-one

To a stirred solution of methyl (2E,4E)-4-(1-methyltetrahydropyrimidin-2(1H)-ylidene)pent-2-enoate (1.5 g, 6.224 mmol, 1.0 eq) in DMF (7 mL) was added Et₃N (1.8 mL, 12.44 mmol, 2.0 eq). The resulting mixture was heated at 150° C. for 12 h. The progress of reaction was monitored by LCMS. The reaction mixture was cooled to rt, diluted with water and extracted in to EtOAc (2×50 mL). The combined organic layers were washed with water (30 mL), with brine (30 mL), dried over Na₂SO₄, concentrated to afford crude desired compound 1-methyl-9-nitro-1,2,3,4-tetrahydro-6H-pyrido [1,2-a]pyrimidin-6-one (0.7 g, 53.84%) as brown solid. LCMS: (M+1)⁺: 210.2

### Step-4: Synthesis of 7-bromo-1-methyl-9-nitro-1,2,3,4-tetrahydro-6H-pyrido[1,2-a]pyrimidin-6-one

To a stirred solution of compound 1-methyl-9-nitro-1,2,3,4-tetrahydro-6H-pyrido[1,2-a]pyrimidin-6-one (2.0 g, 9.5693 mmol, 1.0 eq) in H₂O (25 mL) was added NBS (1.7 g, 9.57 mmol, 1 eq) at 0° C. The resulting mixture was brought to rt and stirred for 4 h. The progress of reaction was monitored by LCMS. The reaction mixture was diluted with water (30 mL), extracted with EtOAc (2×50 mL). The combined organic layers were washed with water (30 mL), with brine (30 mL), dried over Na₂SO₄, concentrated to afford crude desired compound 7-bromo-1-methyl-9-nitro-1,2,3,4-tetrahydro-6H-pyrido[1,2-a]pyrimidin-6-one (1.5 g, 54.54%) as Brown solid. LCMS: (M+1)⁺: 288.0

### Step-5: Synthesis of 7-(5-chloro-2-fluorophenyl)-1-methyl-9-nitro-1,2,3,4-tetrahydro-6H-pyrido[1,2-a]pyrimidin-6-one

To a stirred solution of 7-bromo-1-methyl-9-nitro-1,2,3,4-tetrahydro-6H-pyrido[1,2-a]pyrimidin-6-one (1.5 g, 5.24 mmol, 1 eq) & potassium (5-chloro-2-fluorophenyl)trifluoroborate (2.7 g, 11.53 mmol, 2.2 eq) and 2M Na₂CO₃(5.5 ml, 10.48 mmol, 2 eq) in dioxane (10 mL). The resulting mixture was purged with nitrogen for 10 min followed by addition of Pd₂(dppf)Cl₂.DCM complex (0.428 g, 0.524 mmol, 0.12 eq) and again purged with nitrogen for 10 min. The reaction mixture was heated at 100° C. for overnight. The progress of reaction was monitored by LCMS. The reaction mixture was diluted with water (50 mL), extracted with EtOAc (2×100 mL). The combined organic layers were washed with water (30 mL), with brine (30 mL), dried over Na₂SO₄, concentrated to afford crude, which was purified by column chromatography to afford the desired 7-(5-chloro-2-fluorophenyl)-1-methyl-9-nitro-1,2,3,4-tetrahydro-6H-pyrido[1,2-a]pyrimidin-6-one (600 mg, 35.09%) as yellowish solid. LCMS: (M+1)⁺: 338.2

### Step-6: Synthesis of 9-amino-7-(5-chloro-2-fluorophenyl)-1-methyl-1,2,3,4-tetrahydro-6H-pyrido[1,2-a]pyrimidin-6-one

To a stirred solution of 7-(5-chloro-2-fluorophenyl)-1-methyl-9-nitro-1,2,3,4-tetrahydro-6H-pyrido[1,2-a]pyrimidin-6-one (0.5 g, 1.48 mmol, 1.0 eq) in 60 mL EtOH: H₂O (1:1) was added Fe (0.56 g, 13.0 mmol, 7.0 eq) followed by NH₄Cl (0.580 g, 13.0 mmol, 7.0 eq) at rt. The resulting mixture was stirred at 90° C. for 3 h. The progress of reaction was monitored by LCMS. The reaction mixture was filtered through celite the filtrate was concentrated, diluted with water (30 mL), extracted with EtOAc (2×50 mL). The combined organic layers were washed with brine (50 mL), dried over Na₂SO₄, concentrated to afford the desired compound 9-amino-7-(5-chloro-2-fluorophenyl)-1-methyl-1,2,3,4-tetrahydro-6H-pyrido[1,2-a]pyrimidin-6-one (0.400 g, 87.71%) as green solid. LCMS: (M+1)⁺: 308.2

### Step-7: Synthesis of methyl 4-((7-(5-chloro-2-fluorophenyl)-1-methyl-6-oxo-1,3,4,6-tetrahydro-2H-pyrido[1,2-a]pyrimidin-9-yl)amino)nicotinate

To a stirred solution of 9-amino-7-(5-chloro-2-fluorophenyl)-1-methyl-1,2,3,4-tetrahydro-6H-pyrido[1,2-a]pyrimidin-6-one (0.50 g, 1.623 mmol, 1 eq) & methyl 4-chloronicotinate (0.310 g, 1.786 mmol, 1.1 eq) and CS₂CO₃(1.05 g, 3.246 mmol, 2 eq) in dioxane (10.0 mL). The resulting mixture was purged with nitrogen for 10 min followed by addition of Pd₂(dba)₃ (0.15 g, 0.162 mmol, 0.12 eq) and xantphos (0.188 g, 0.325 mmol, 0.2 eq), again purged with nitrogen for 10 min. The reaction mixture was heated at 100° C. for overnight. The progress of reaction was monitored by LCMS. The reaction mixture was diluted with water (30 mL), extracted with EtOAc (2×50 mL). The combined organic layers were washed with water (30 mL), with brine (30 mL), dried over Na₂SO₄, concentrated to afford the desired methyl 4-((7-(5-chloro-2-fluorophenyl)-1-methyl-6-oxo-1,3,4,6-tetrahydro-2H-pyrido[1,2-a]pyrimidin-9-yl)amino)nicotinate, (400 mg, 55.63%) as brown solid. LCMS: (M+1)⁺: 443.3

### Step-8: Synthesis of lithium 4-((7-(5-chloro-2-fluorophenyl)-1-methyl-6-oxo-1,3,4,6-tetrahydro-2H-pyrido[1,2-a]pyrimidin-9-yl)amino)nicotinate

To a stirred solution of methyl 4-((7-(5-chloro-2-fluorophenyl)-1-methyl-6-oxo-1,3,4,6-tetrahydro-2H-pyrido[1,2-a]pyrimidin-9-yl)amino)nicotinate (0.35 g, 0.7918 mmol, 1.0 eq) in THF (6 mL). Was added a solution of LiOH.H₂O (0.04 g, 1.5837 mmol, 2.0 eq) in water (2.0 mL) at rt. The reaction mixture was stirred at for 2 h. The progress of reaction was monitored by LCMS. The reaction mixture was concentrated to & triturate with Hexane afford the desired lithium 4-((7-(5-chloro-2-fluorophenyl)-1-methyl-6-oxo-1,3,4,6-tetrahydro-2H-pyrido[1,2-a]pyrimidin-9-yl)amino)nicotinate (300 mg, 88.75%) as Brown solid. LCMS: (M+1)⁺429.3

### Step-9: Synthesis of (S)-4-((7-(5-chloro-2-fluorophenyl)-1-methyl-6-oxo-1,3,4,6-tetrahydro-2H-pyrido[1,2-a]pyrimidin-9-yl)amino)-N-(2-hydroxypropyl)nicotinamide

To a stirred solution of lithium 4-((7-(5-chloro-2-fluorophenyl)-1-methyl-6-oxo-1,3,4,6-tetrahydro-2H-pyrido[1,2-a]pyrimidin-9-yl)amino)nicotinate (0.060 g, 0.1401 mmol, 1.0 eq) in DMF (2 mL) was added HATU (0.080 g, 0.2101 mmol, 1.5 eq) at rt. The resulting mixture was stirred at rt for 5 minutes, followed by addition of (S)-1-aminopropan-2-ol (0.05 ml, 0.2803 mmol, 1.5 eq) and DIPEA (0.1 mL, 0.4203 mmol, 3.0 eq) and stirred at rt for 1 h. Reaction mixture was diluted with water (20 mL) and extracted with ethyl acetate (2×50 mL) The combined organic extracts were washed with water (50 mL), with brine (50 mL) dried over Na₂SO₄ and concentrated under reduced pressure and purified by prep purification to afford the desired compound (S)-4-((7-(5-chloro-2-fluorophenyl)-1-methyl-6-oxo-1,3,4,6-tetrahydro-2H-pyrido[1,2-a]pyrimidin-9-yl)amino)-N-(2-hydroxypropyl)nicotinamide (16 mg, 12.0%) as off white solid. LCMS: (M+1)⁺486.2. UPLC: At 254 nm: 98.40%, At 220 nm: 96.54%.

¹H NMR (400 MHz, METHANOL-d₄): δ 9.49 (m, 1H), 8.38 (d, 1H), 8.14 (d, J=5.7 Hz, 1H), 7.56-7.50 (m, 1H), 7.29-7.19 (m, 2H), 7.12-7.00 (m, 2H), 6.60 (d, J=5.7 Hz, 1H), 4.49 (br. s., 1H), 4.21-4.11 (m, 2H), 3.97 (dd, J=6.6, 11.8 Hz, 2H), 3.70 (m, 1H), 3.44-3.34 (m, 2H), 3.05 (s, 3H), 2.21-2.10 (m, 2H), 1.21 (d, J=6.1 Hz, 3H).

### Example-21: Synthesis of (S)-4-((7-(5-chloro-2-fluorophenyl)-1-methyl-6-oxo-1,3,4,6-tetrahydro-2H-pyrido[, 2-a]pyrimidin-9-yl)amino)-N-(1-hydroxypropan-2-yl)nicotinamide: (Compound 1.21)

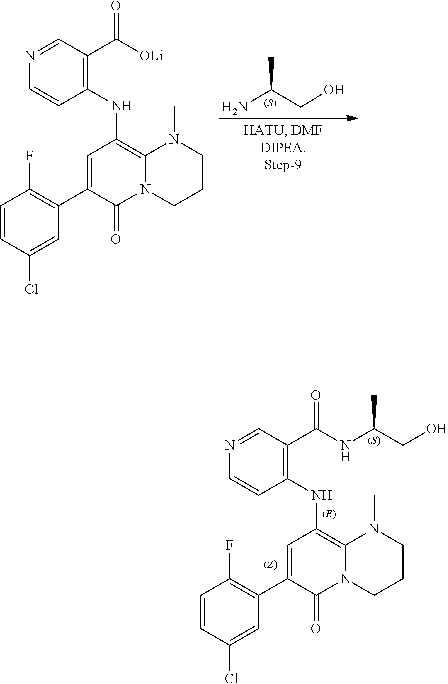

Steps 1 to 8 are the same as in Example 20.

### Step-9: Synthesis of (S)-4-((7-(5-chloro-2-fluorophenyl)-1-methyl-6-oxo-1,3,4,6-tetrahydro-2H-pyrido[1,2-a]pyrimidin-9-yl)amino)-N-(1-hydroxypropan-2-yl)nicotinamide

To a stirred solution of lithium 4-((7-(5-chloro-2-fluorophenyl)-1-methyl-6-oxo-1,3,4,6-tetrahydro-2H-pyrido[1,2-a]pyrimidin-9-yl)amino)nicotinate (0.060 g, 0.14 mmol, 1.0 eq) in DMF (2 mL) was added HATU (0.080 g, 0.21 mmol, 1.5 eq) at rt. The resulting mixture was stirred at rt for 5 minutes, followed by addition of (S)-2-aminopropan-2-ol (0.05 ml, 0.28 mmol, 1.5 eq) and DIPEA (0.1 mL, 0.42 mmol, 3.0 eq) and stirred at rt for 1 h. Reaction mixture was diluted with water (20 mL) and extracted with ethyl acetate (2×50 mL) The combined organic extracts were washed with water (50 mL), with brine (50 mL) dried over Na₂SO₄ and concentrated under reduced pressure and purified by prep purification to afford the desired compound (S)-4-((7-(5-chloro-2-fluorophenyl)-1-methyl-6-oxo-1,3,4,6-tetrahydro-2H-pyrido[1 1,2-a]pyrimidin-9-yl)amino)-N-(1-hydroxypropan-2-yl)nicotinamide (12 mg, 10.0%) as off yellow solid. LCMS: (M+1)⁺486.2, UPLC: At 254 nm: 100%, At 220 nm: 100%.

¹H NMR (400 MHz, METHANOL-d₄): δ 9.49 (m, 1H), 8.72 (s, 1H), 8.57 (s, 1H), 8.14 (d, J=6.1 Hz, 1H), 7.55 (dd, J=2.9, 6.4 Hz, 1H), 7.29-7.21 (m, 1H), 7.12-7.04 (m, 2H), 6.61 (d, J=5.7 Hz, 1H), 4.49 (t, 1H), 4.21 (d, J=6.1 Hz, 2H), 4.14 (d, J=4.4 Hz, 2H), 3.70 9m, 1H),3.60 (d, J=5.7 Hz, 2H), 3.05 (s, 3H), 2.18-2.07 (m, 2H), 1.33-1.24 (m, 3H).

### Example-22: Synthesis of (S)-4-((6-(5-chloro-2-fluorophenyl)-1-methyl-5-oxo-1,2,3,5-tetrahydroimidazo[1,2-a]pyridin-8-yl)amino)-N-(1-hydroxypropan-2-yl)nicotinamide: (Compound 1.22)

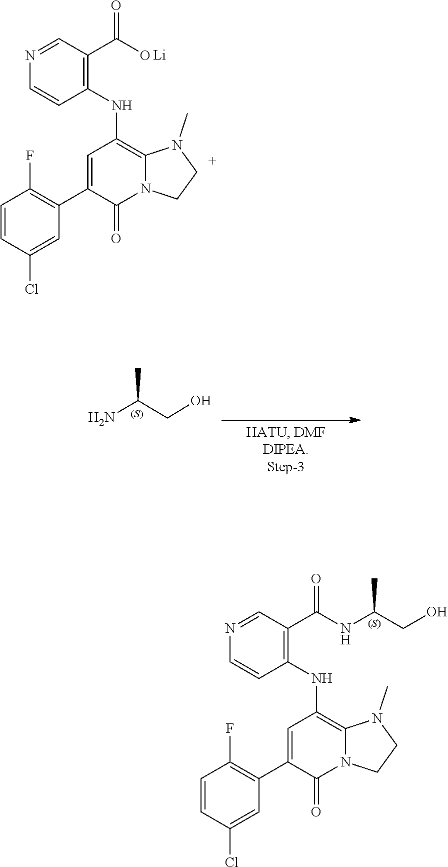

Steps 1 to 2 are the same as in Example 19.

### Step-3: Synthesis of (S)-4-((6-(5-chloro-2-fluorophenyl)-1-methyl-5-oxo-1,2,3,5-tetrahydroimidazo[1,2-a]pyridin-8-yl)amino)-N-(1-hydroxypropan-2-yl)nicotinamide

To a stirred solution of lithium 4-((6-(5-chloro-2-fluorophenyl)-1-methyl-5-oxo-1,2,3,5-tetrahydroimidazo[1,2-a]pyridin-8-yl)amino)nicotinate (0.050 g, 0.1207 mmol, 1.0 eq) in DMF (2 mL) was added HATU (0.069 g, 0.1810 mmol, 1.5 eq) at rt. The resulting mixture was stirred at rt for 5 minutes, followed by addition of (S)-2-aminopropan-2-ol (0.05 ml, 0.2415 mmol, 1.5 eq) and DIPEA (0.1 mL, 0.3621 mmol, 3.0 eq) and stirred at rt for 1 h. Reaction mixture was diluted with water (20 mL) and extracted with ethyl acetate (2×50 mL) The combined organic extracts were washed with water (50 mL), with brine (50 mL) dried over Na₂SO₄ and concentrated under reduced pressure and purified by prep purification to afford the desired compound (S)-4-((6-(5-chloro-2-fluorophenyl)-1-methyl-5-oxo-1,2,3,5-tetrahydroimidazo[1,2-a]pyridin-8-yl)amino)-N-(1-hydroxypropan-2-yl)nicotinamide (13 mg, 10.0%) as off white solid. LCMS: (M+1)⁺472.2, UPLC: —At 254 nm: 97.79%, At 220 nm: 98.47%.

¹H NMR (400 MHz, METHANOL-d₄): δ 9.49 (m, 1H), 8.72 (s, 1H), 8.57 (s, 1H), 8.15 (d, J=5.7 Hz, 1H), 7.58-7.50 (m, 1H), 7.35 (s, 1H), 7.16-7.02 (m, 2H), 6.66 (d, J=5.7 Hz, 1H), 4.49 (t, 1H), 4.21 (t, J=9.0 Hz, 2H), 3.75 (t, J=9.0 Hz, 2H), 3.70 (m, 1H), 3.60 (d, J=5.7 Hz, 2H), 3.02 (s, 3H), 1.30-1.11 (m, 3H).

### Example-23: Synthesis of (R)-4-((6-(5-chloro-2-fluorophenyl)-1-methyl-5-oxo-1,2,3,5-tetrahydroimidazo[1,2-a]pyridin-8-yl)amino)-N-(1-hydroxypropan-2-yl)nicotinamide: (Compound 1.23)

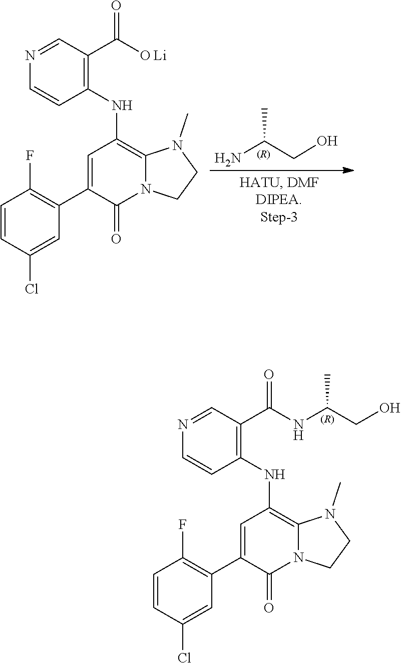

Steps 1 to 2 are the same as in Example 19.

### Step-3: Synthesis of (R)-4-((6-(5-chloro-2-fluorophenyl)-1-methyl-5-oxo-1,2,3,5-tetrahydroimidazo[1,2-a]pyridin-8-yl)amino)-N-(1-hydroxypropan-2-yl)nicotinamide

To a stirred solution of lithium 4-((6-(5-chloro-2-fluorophenyl)-1-methyl-5-oxo-1,2,3,5-tetrahydroimidazo[1,2-a]pyridin-8-yl)amino)nicotinate (0.05 g, 0.1207 mmol, 1.0 eq) in DMF (2 mL) was added HATU (0.069 g, 0.181 mmol, 1.5 eq) at rt. The resulting mixture was stirred at rt for 5 minutes, followed by addition of (R)-2-aminopropan-1-ol (0.05 ml, 0.241 mmol, 1.5 eq) and DIPEA (0.1 mL, 0.362 mmol, 3.0 eq) and stirred at rt for 1 h. Reaction mixture was diluted with water (20 mL) and extracted with ethyl acetate (2×50 mL) The combined organic extracts were washed with water (50 mL), with brine (50 mL) dried over Na₂SO₄ and concentrated under reduced pressure and purified by prep purification to afford the desired compound (R)-4-((6-(5-chloro-2-fluorophenyl)-1-methyl-5-oxo-1,2,3,5-tetrahydroimidazo[1,2-a]pyridin-8-yl)amino)-N-(1-hydroxypropan-2-yl)nicotinamide (25 mg, 15.0%) as yellow solid. LCMS: (M+1)⁺472.2, UPLC: At 254 nm: 99.95%, At 220 nm: 99.92%.

¹H NMR (400 MHz, METHANOL-d₄): δ 9.49 (m, 1H), 8.72 (s, 1H), 8.59 (s, 1H), 8.16 (d, J=6.6 Hz, 1H), 7.56 (dd, J=2.6, 6.6 Hz, 1H), 7.35 (s, 1H), 7.25 (dd, J=4.4, 7.0 Hz, 1H), 7.13-7.03 (m, 1H), 6.70 (d, J=6.1 Hz, 1H), 4.49 (d, 1H), 4.21-4.14 (m, 2H), 3.76 (t, J=9.0 Hz, 2H), 3.70 (m, 1H), 3.60 (d, J=5.7 Hz, 2H), 3.02 (s, 3H), 1.30-1.18 (m, 3H).

### Example-24: Synthesis of (R)-4-((6-(5-chloro-2-fluorophenyl)-1-methyl-5-oxo-1,2,3,5-tetrahydroimidazo[1,2-a]pyridin-8-yl)amino)-N-(2-hydroxypropyl)nicotinamide: (Compound 1.24)

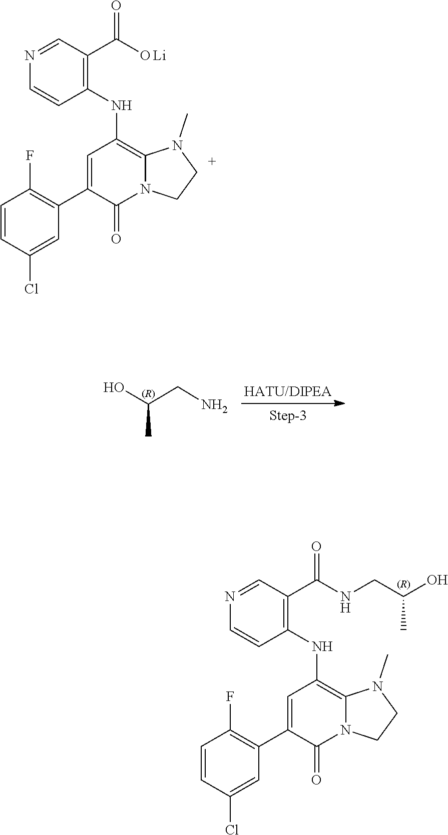

Steps 1 to 2 are the same as in Example 19.

### Step-3: Synthesis of (R)-4-((6-(5-chloro-2-fluorophenyl)-1-methyl-5-oxo-1,2,3,5-tetrahydroimidazo[,2-a]pyridin-8-yl)amino)-N-(2-hydroxypropyl)nicotinamide

To a stirred solution of lithium 4-((6-(5-chloro-2-fluorophenyl)-1-methyl-5-oxo-1,2,3,5-tetrahydroimidazo[1,2-a]pyridin-8-yl)amino)nicotinate (0.050 g, 0.121 mmol, 1.0 eq) in DMF (2 mL) was added HATU (0.069 g, 0.181 mmol, 1.5 eq) at rt. The resulting mixture was stirred at rt for 5 minutes, followed by addition of (R)-1-aminopropan-2-ol (0.05 ml, 0.241 mmol, 1.5 eq) and DIPEA (0.1 mL, 0.362 mmol, 3.0 eq) and stirred at rt for 1 h. Reaction mixture was diluted with water (20 mL) and extracted with ethyl acetate (2×50 mL) The combined organic extracts were washed with water (50 mL), with brine (50 mL) dried over Na₂SO₄ and concentrated under reduced pressure and purified by prep purification to afford the desired compound (R)-4-((6-(5-chloro-2-fluorophenyl)-1-methyl-5-oxo-1,2,3,5-tetrahydroimidazo[1,2-a]pyridin-8-yl)amino)-N-(2-hydroxypropyl)nicotinamide (23 mg, 13%) as off yellow solid. LCMS: (M+1)⁺472.2. UPLC: At 254 nm: 99.90%, At 220 nm: 99.62%.

¹H NMR (400 MHz, METHANOL-d₄): δ 9.49 (t, 1H), 8.59 (s, 1H), 8.38 (d, 1H), 8.16 (d, J=5.7 Hz, 1H), 7.56 (dd, J=2.6, 6.1 Hz, 1H), 7.31-7.18 (m, 1H), 7.15-7.04 (m, 2H), 6.67 (d, J=6.1 Hz, 1H), 5.37 (d, 1H), 4.21 (t, J=9.0 Hz, 2H), 3.78 (m, 1H), 3.75 (t, J=9.2 Hz, 2H), 3.44-3.35 (m, 2H), 3.02 (s, 3H), 1.21 (d, J=6.1 Hz, 3H).

### Example-25: Synthesis of (R)-4-((7-(5-chloro-2-fluorophenyl)-1-methyl-6-oxo-1,3,4,6-tetrahydro-2H-pyrido[, 2-a]pyrimidin-9-yl)amino)-N-(1-hydroxypropan-2-yl)nicotinamide: (Compound 1.25)

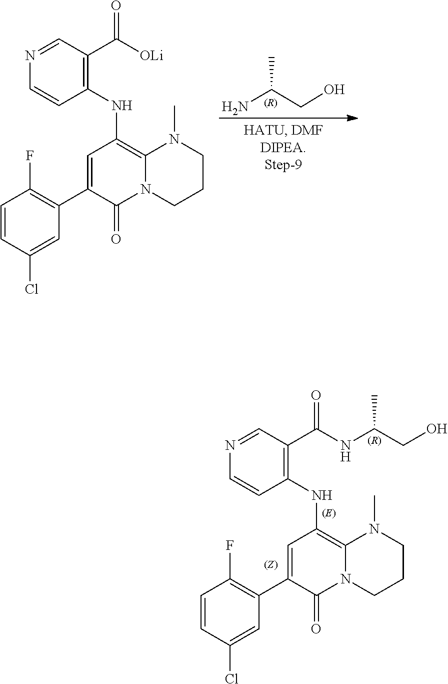

Steps 1 to 8 are the same as in Example 20.

### Step-9: Synthesis of (R)-4-((7-(5-chloro-2-fluorophenyl)-1-methyl-6-oxo-1,3,4,6-tetrahydro-2H-pyrido[1,2-a]pyrimidin-9-yl)amino)-N-(1-hydroxypropan-2-yl)nicotinamide

To a stirred solution of lithium 4-((7-(5-chloro-2-fluorophenyl)-1-methyl-6-oxo-1,3,4,6-tetrahydro-2H-pyrido[1,2-a]pyrimidin-9-yl)amino)nicotinate (0.06 g, 0.14 mmol, 1.0 eq) in DMF (2 mL) was added HATU (0.08 g, 0.21 mmol, 1.5 eq) at rt. The resulting mixture was stirred at rt for 5 minutes, followed by addition of (R)-2-aminopropan-1-ol (0.05 ml, 0.28 mmol, 1.5 eq) and DIPEA (0.1 mL, 0.42 mmol, 3.0 eq) and stirred at rt for 1 h. Reaction mixture was diluted with water (20 mL) and extracted with ethyl acetate (2×50 mL) The combined organic extracts were washed with water (50 mL), with brine (50 mL) dried over Na₂SO₄ and concentrated under reduced pressure and purified by prep purification to afford the desired compound (R)-4-((7-(5-chloro-2-fluorophenyl)-1-methyl-6-oxo-1,3,4,6-tetrahydro-2H-pyrido[1,2-a]pyrimidin-9-yl)amino)-N-(1-hydroxypropan-2-yl)nicotinamide (09 mg, 10.0%) as yellow solid. LCMS: (M+1)⁺486.3, UPLC: At 254 nm: 98.96%, At 220 nm: 99.11%.

¹H NMR (400 MHz, METHANOL-d₄): δ 9.49 (m, 1H), 8.56 (s, 1H), 8.13 (d, J=5.7 Hz, 1H), 7.60-7.53 (m, 2H), 7.38 (dd, 1H), 7.15-7.07 (m, 2H), 6.57 (d, J=6.1 Hz, 1H), 4.49 (t, 1H), 4.14 (d, 2H), 3.70 (m, 1H), 3.60 (d, J=5.7 Hz, 2H), 3.05 (s, 3H), 2.14 (dd, 2H), 1.94 (d, 2H), 1.38-1.15 (m, 3H).

### Example-26: Synthesis of (R)-4-((7-(5-chloro-2-fluorophenyl)-1-methyl-6-oxo-1,3,4,6-tetrahydro-2H-pyrido[1,2-a]pyrimidin-9-yl)amino)-N-(2-hydroxypropyl)nicotinamide (Compound 1.26)

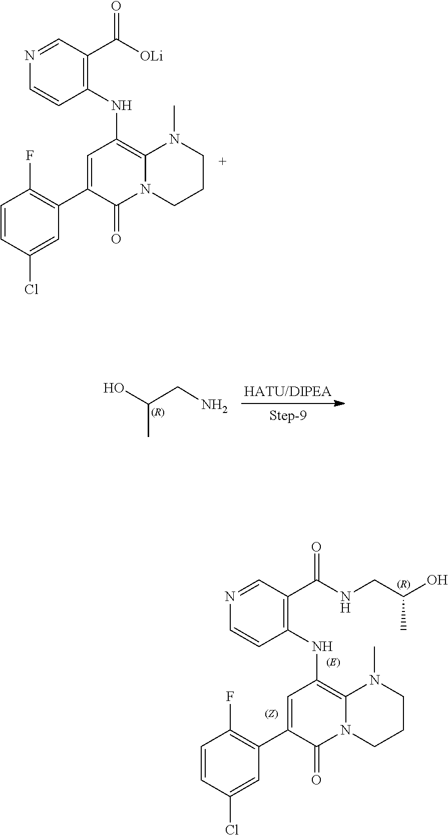

Steps 1 to 8 are the same as in Example 20.

### Step-9: Synthesis of (R)-4-((7-(5-chloro-2-fluorophenyl)-1-methyl-6-oxo-1,3,4,6-tetrahydro-2H-pyrido[1,2-a]pyrimidin-9-yl)amino)-N-(2-hydroxypropyl)nicotinamide

To a stirred solution of lithium 4-((7-(5-chloro-2-fluorophenyl)-1-methyl-6-oxo-1,3,4,6-tetrahydro-2H-pyrido [1,2-a]pyrimidin-9-yl)amino)nicotinate (0.06 g, 0.14 mmol, 1.0 eq) in DMF (2 mL) was added HATU (0.08 g, 0.2101 mmol, 1.5 eq) at rt. The resulting mixture was stirred at rt for 5 minutes, followed by addition of (R)-1-aminopropan-2-ol (0.05 ml, 0.28 mmol, 1.5 eq) and DIPEA (0.1 mL, 0.42 mmol, 3.0 eq) and stirred at rt for 1 h. Reaction mixture was diluted with water (20 mL) and extracted with ethyl acetate (2×50 mL) The combined organic extracts were washed with water (50 mL), brine (50 mL) dried over Na₂SO₄ and concentrated under reduced pressure and purified by prep purification to afford the desired compound (R)-4-((7-(5-chloro-2-fluorophenyl)-1-methyl-6-oxo-1,3,4,6-tetrahydro-2H-pyrido[1,2-a]pyrimidin-9-yl)amino)-N-(2-hydroxypropyl)nicotinamide (20 mg, 15%) as yellow solid. LCMS: (M+1)⁺486.2. UPLC: At 254 nm: 96.70%, At 220 nm: 94.15%.

¹H NMR (400 MHz, METHANOL-d₄): δ 9.49 (m, 1H), 8.60 (s, 1H), 8.15 (d, J=6.1 Hz, 1H), 7.55 (dd, J=2.6, 6.1 Hz, 2H), 7.26 (d, J=3.9 Hz, 2H), 7.10 (d, J=9.2 Hz, 1H), 6.67 (d, J=5.7 Hz, 1H), 5.37 (t, 1H), 4.14 (d, J=5.7 Hz, 2H), 3.97 (d, J=4.8 Hz, 2H), 3.78 (m, 1H), 3.51-3.40 (m, 2H), 3.05 (s, 3H), 2.15 (dd, 2H), 1.22 (d, J=6.1 Hz, 3H).

### Example-27: Synthesis of (S)-4-((6′-(dimethylamino)-6-methyl-2′-oxo-1′,2′-dihydro-[2,3′-bipyridin]-5′-yl)amino)-N-(2-hydroxypropyl)nicotinamide: (Compound 1.27)

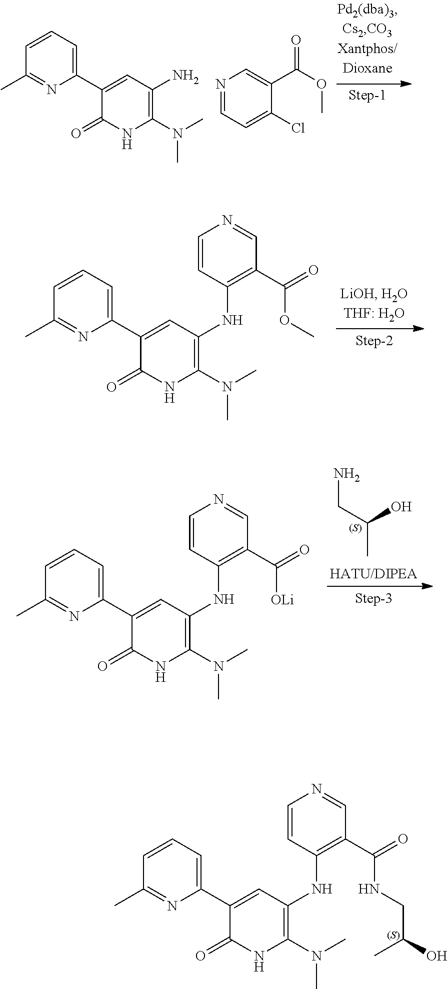

### Step-1: Synthesis of methyl 4-((6′-(dimethylamino)-6-methyl-2′-oxo-1′,2′-dihydro-[2,3′-bipyridin]-5′-yl)amino)nicotinate

To a stirred solution of compound 5′-amino-6′-(dimethylamino)-6-methyl-[2,3′-bipyridin]-2′(1′H)-one (700 mg, 2.865 mmol, 1.0 eq) and methyl 4-chloronicotinate (540 mg, 3.151 mmol, 1.1 eq) in dioxane (30 mL) was added Cs₂CO₃ (3.74 g, 11.46 mmol, 4.0 eq) at rt. The resulting mixture was purged with nitrogen for 10 min followed by addition of Pd₂(dba)₃ (315 mg, 0.343 mmol, 0.12 eq) and xantphos (248 mg, 0.429 mmol, 0.15 eq), again purged with nitrogen for 10 min. The reaction mixture was heated at 100° C. for overnight. The progress of reaction was monitored by LCMS. The reaction mixture was diluted with water (50 mL), extracted with EtOAc (2×100 mL). The combined organic layers were washed with water (50 mL), with brine (50 mL), dried over Na₂SO₄, concentrated and purified by combi flash chromatography [silica gel 100-200 mesh: elution 0-30% EtOAc in Hexane] to afford the desired compound methyl 4-((6′-(dimethylamino)-6-methyl-2′-oxo-1′,2′-dihydro-[2,3′-bipyridin]-5′-yl)amino)nicotinate (170 mg, 15.64%) as brown solid. LCMS: (M+1)⁺: 380.3

### Step-2: Synthesis of lithium 4-((6′-(dimethylamino)-6-methyl-2′-oxo-1′,2′-dihydro-[2,3′-bipyridin]-5′-yl)amino)nicotinate

To a stirred solution of methyl 4-((6′-(dimethylamino)-6-methyl-2′-oxo-1′,2′-dihydro-[2,3′-bipyridin]-5′-yl)amino)nicotinate (170 mg, 0.448 mmol, 1.0 eq) in THF (8.0 mL) was added a solution of LiOH.H₂O (38 mg, 0.896 mmol, 2.0 eq) in water (2.0 mL) at rt. The reaction mixture was stirred at for overnight. The progress of reaction was monitored by LCMS. The reaction mixture was concentrated to afford the desired lithium 4-((6′-(dimethylamino)-6-methyl-2′-oxo-1′,2′-dihydro-[2,3′-bipyridin]-5′-yl)amino)nicotinate (165 mg, 99.39%) as brown solid. LCMS: (M+1)⁺: 366.2

### Step-3: Synthesis of (S)-4-((6′-(dimethylamino)-6-methyl-2′-oxo-1′,2′-dihydro-[2,3′-bipyridin]-5′-yl)amino)-N-(2-hydroxypropyl)nicotinamide

To a stirred solution of lithium 4-((6′-(dimethylamino)-6-methyl-2′-oxo-1′,2′-dihydro-[2,3′-bipyridin]-5′-yl)amino)nicotinate (165 mg, 0.444 mmol, 1.0 eq) in DMF (5 mL) was added HATU (253 mg, 0.666 mmol, 1.5 eq) at rt. The resulting mixture was stirred at rt for 5 minutes, followed by addition of (S)-1-aminopropan-2-ol (66 mg, 0.888 mmol, 2.0 eq) and DIPEA (0.23 mL, 1.332 mmol, 3.0 eq) and stirred at rt for 30 min. Reaction mixture was diluted with water (50 mL) and extracted with ethyl acetate (2×50 mL). The combined organic extracts were washed with water (50 mL), with brine (50 mL) dried over Na₂SO₄ and concentrated under reduced and purified by combi flash chromatography [silic gel 100-200 mesh; elution 0-10% MeOH in DCM] to afford the desired compound (S)-4-((6′-(dimethylamino)-6-methyl-2′-oxo-1′,2′-dihydro-[2,3′-bipyridin]-5′-yl)amino)-N-(2-hydroxypropyl)nicotinamide (15 mg, 8.19%) as yellow solid. LCMS: (M+1)⁺423.3, UPLC: At 254 nm: 92.02%, At 220 nm: 91.38%.

¹H NMR (400 MHz, DMSO-d₆): δ 15.38 (brs, 1H), 10.02 (brs, 1H), 8.79 (brs, 1H), 8.71 (s, 1H), 8.10-8.21 (m, 2H), 7.87 (brs, 1H), 7.79 (t, J=7.89 Hz, 1H), 7.38 (brs, 1H), 7.18 (d, J=7.89 Hz, 1H), 6.48 (d, J=6.58 Hz, 1H), 4.81 (d, J=5.26 Hz, 1H), 3.76-3.86 (m, 1H), 3.11-3.27 (m, 3H), 2.76-3.05 (m, 6H), 2.54 (brs, 3H), 1.10 (d, J=6.14 Hz, 3H).

### Example-28: Synthesis of 3-(5-chloro-2-fluorophenyl)-6-(dimethylamino)-5-((1-methyl-1H-pyrrolo[2,3-b]pyridin-4-yl)amino)pyridin-2(1H)-one (Compound 1.28)

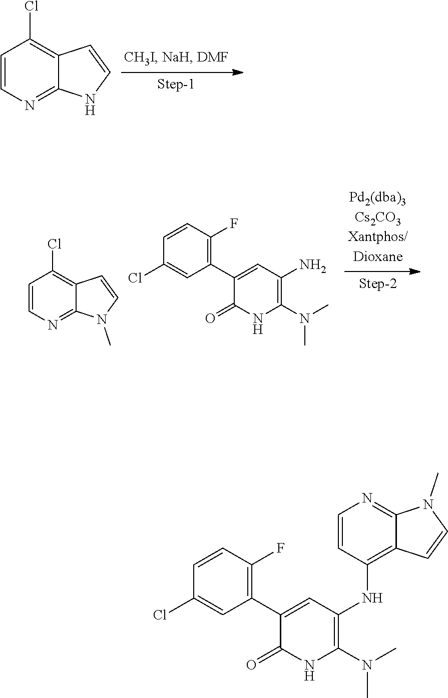

### Step-1-: Synthesis of 4-chloro-1-methyl-1H-pyrrolo[2,3-b]pyridine

To a stirred solution of 4-chloro-1H-pyrrolo[2,3-b]pyridine (1.0 g, 6.554 mmol, 1.0 eq) in DMF (20 mL) was added NaH (60%) (0.53 g, 13.188 mmol, 2.0 eq) at 0° C. The resulting mixture was allowed to stirred at the same temperature for 5 min followed by addition of CH₃I (0.45 mL, 7.209 mmol, 1.1 eq). The resulting mixture was stirred at rt for 15 min. The progress of reaction was monitored by LCMS. The reaction mixture was poured into ice cold water (50 mL), extracted with EtOAc (2×50 mL), the combined organic layers were washed with water (50 mL), with brine (50 mL), dried over Na₂SO₄, concentrated to afford the desired compound (1.05 g, 96.33%) as brown liquid. LCMS: (M+1)⁺167.0

### Step-2: Synthesis of 3-(5-chloro-2-fluorophenyl)-6-(dimethylamino)-5-((1-methyl-1H-pyrrolo[2,3-b]pyridin-4-yl)amino)pyridin-2(1H)-one

To a stirred solution of compound 5-amino-3-(5-chloro-2-fluorophenyl)-6-(dimethylamino)pyridin-2(1H)-one (0.20 g, 0.710 mmol, 1.0 eq) and 4-chloro-1-methyl-1H-pyrrolo[2,3-b]pyridine (0.142 g, 0.852 mmol, 1.2 eq) in dioxane (10 mL) was added Cs₂CO₃ (0.925 g, 2.840 mmol, 4.0 eq) at rt. The resulting mixture was purged with nitrogen for 10 min followed by addition of Pd₂(dba)₃ (0.078 g, 0.0852 mmol, 0.12 eq) and xantphos (0.061 g, 0.106 mmol, 0.15 eq), again purged with nitrogen for 10 min. The reaction mixture was heated at 100° C. for overnight. The progress of reaction was monitored by LCMS. The reaction mixture was diluted with water (50 mL), extracted with EtOAc (2×50 mL). The combined organic layers were washed with water (50 mL), with brine (50 mL), dried over Na₂SO₄, concentrated and purified by reverse phase chromatography to afford the desired compound (0.008 g, 2.74%) as brown solid. LCMS: (M+1)⁺412.2; ¹H NMR (400 MHz, DMSO-d₆): δ 10.69 (brs, 1H), 8.03 (brs, 1H), 7.83 (s, 1H), 7.55 (brs, 1H), 7.33 (s, 2H), 7.19-7.29 (m, 2H), 7.14 (d, J=3.51 Hz, 1H), 6.49 (d, J=3.51 Hz, 1H), 5.97 (d, J=5.26 Hz, 1H), 3.71 (s, 3H), 2.92 (s, 6H).

### Example-29: Synthesis methyl 4-((5-(5-chloro-2-fluorophenyl)-2-(dimethylamino)-6-hydroxypyridin-3-yl)amino)nicotinate (Compound 1.29)

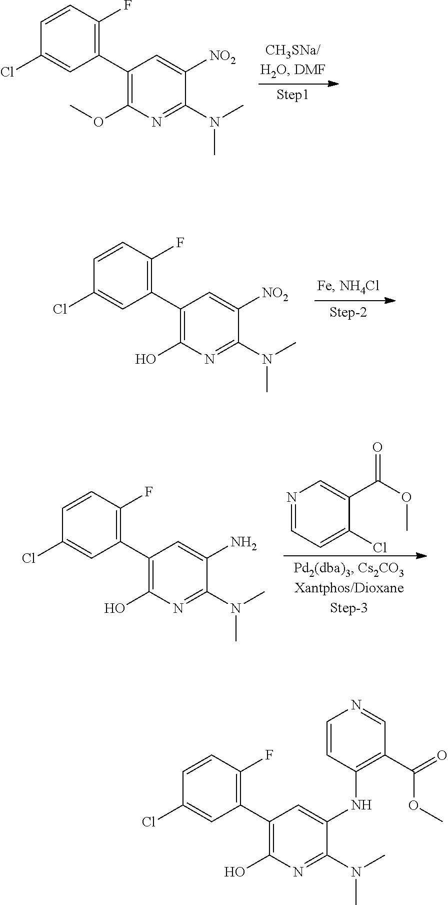 [SMILES: COc1nc(N(C)C)c([N+](=O)[O-])cc1-c1cc(Cl)ccc1F ; CN(C)c1nc(O)c(-c2cc(Cl)ccc2F)cc1[N+](=O)[O-] ; CN(C)c1nc(O)c(-c2cc(Cl)ccc2F)cc1N ; COC(=O)c1cnccc1Cl ; COC(=O)c1cnccc1Nc1cc(-c2cc(Cl)ccc2F)c(O)nc1N(C)C]

### Step-1: Synthesis of 3-(5-chloro-2-fluorophenyl)-6-(dimethylamino)-5-nitropyridin-2-ol

To a stirred solution of 5-(5-chloro-2-fluorophenyl)-6-methoxy-N,N-dimethyl-3-nitropyridin-2-amine (4.0 g, 12.30 mmol, 1.0 eq) in DMF (30 mL) was added 21% solution of CH₃SNa (2.23 g, 31.99 mmol, 4.0 eq) in water (12.0 mL) at rt. The reaction mixture was stirred at 150° C. for overnight. The progress of reaction was monitored by LCMS. The reaction mixture was acidified with 1N HCl (5.0 mL). The reaction mixture was poured in ice cold water (500 mL), filtered and the residue was washed with water (500 mL), dried under vacuum to afford the desired compound (3.2 g, 84.21%) as green solid. LCMS: (M+1)⁺312.2

### Step-2: Synthesis of 5-amino-3-(5-chloro-2-fluorophenyl)-6-(dimethylamino)pyridin-2-ol

To a stirred solution of 3-(5-chloro-2-fluorophenyl)-6-(dimethylamino)-5-nitropyridin-2-ol (3.2 g, 10.28 mmol, 1.0 eq) in EtOH (60 mL) and H₂O (60 ml) was added Fe (4.6 g, 82.31 mmol, 8.0 eq) and a solution of NH₄Cl (5.5 g, 102.89 mmol, 10.0 eq) at rt. The resulting mixture was heated at 90° C. for 60 min. The progress of reaction was monitored by LCMS. The reaction mixture was filtered through celite the residue was washed with EtOH (50 mL) the filtrate was concentrated and the residue was dissolved in EtOAc (200 mL), washed with water (2×100 mL), dried over Na₂SO₄, and concentrated to afford the desired compound (2.5 g, 86.20%) as brown solid. LCMS: (M+1)⁺282.2

### Step-3: Synthesis of methyl 4-((5-(5-chloro-2-fluorophenyl)-2-(dimethylamino)-6-hydroxypyridin-3-yl)amino)nicotinate

To a stirred solution of compound 5-amino-3-(5-chloro-2-fluorophenyl)-6-(dimethylamino)pyridin-2-ol (0.450 g, 1.59 mmol, 1.0 eq) and methyl 4-chloronicotinate (0.301 g, 1.75 mmol, 1.0 eq) in dioxane (5.0 mL) was added Cs₂CO₃ (2.08 g, 6.38 mmol, 4.0 eq) at rt. The resulting mixture was purged with nitrogen for 10 min followed by addition of Pd₂(dba)₃ (0.175 g, 0.191 mmol, 0.12 eq) and xantphos (0.138 g, 0.239 mmol, 0.15 eq), again purged with nitrogen for 10 min. The reaction mixture was heated at 100° C. for overnight. The progress of reaction was monitored by LCMS. The reaction mixture was diluted with water (50 mL), extracted with EtOAc (2×100 mL). The combined organic layers were washed with water (50 mL), with brine (50 mL), dried over Na₂SO₄, concentrated and purified by combi flash chromatography [silica gel 100-200 mesh: elution 0-30% EtOAc in Hexane] to afford the desired compound (150 mg, 22.52%) as brown solid. LCMS: (M+1)⁺: 417.2.

### Example-30: Synthesis of 4-((5-(5-chloro-2-fluorophenyl)-2-(dimethylamino)-6-hydroxypyridin-3-yl)amino)-N-cyclopropylnicotinamide (Compound 1.30)

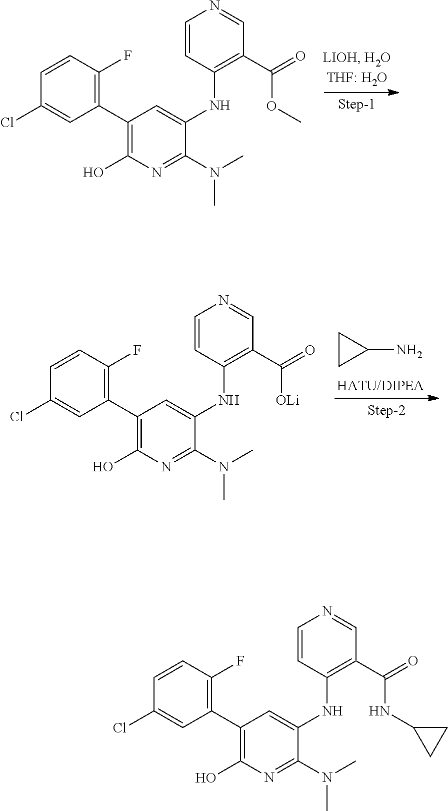

### Step-1: Synthesis lithium 4-((5-(5-chloro-2-fluorophenyl)-2-(dimethylamino)-6-hydroxypyridin-3-yl)amino)nicotinate

To a stirred solution of methyl 4-((5-(5-chloro-2-fluorophenyl)-2-(dimethylamino)-6-hydroxypyridin-3-yl)amino)nicotinate (1.2 g, 2.87 mmol, 1.0 eq) in THF (28.0 mL) was added a solution of LiOH.H₂O (0.242 g, 5.75 mmol, 2.0 eq) in water (7.0 mL) at rt. The reaction mixture was stirred at for overnight. The progress of reaction was monitored by LCMS. The reaction mixture was concentrated to afford the desired compound (1.0 g, 90.9%) as brown solid. LCMS: (M+1)⁺: 403.1

### Step-2: Synthesis of 4-((5-(5-chloro-2-fluorophenyl)-2-(dimethylamino)-6-hydroxypyridin-3-yl)amino)-N-cyclopropylnicotinamide

To a stirred solution of lithium 4-((5-(5-chloro-2-fluorophenyl)-2-(dimethylamino)-6-hydroxypyridin-3-yl) amino)nicotinate (0.250 g, 0.613 mmol, 1.0 eq) in DMF (3 mL) was added HATU (0.349 g, 0.919 mmol, 1.5 eq) at rt. The resulting mixture was stirred at rt for 5 minutes, followed by addition of cyclopropanamine (0.06 ml, 0.919 mmol, 1.5 eq) and DIPEA (0.3 mL, 1.8 mmol, 3.0 eq) and stirred at rt for 30 min. Reaction mixture was diluted with water (50 mL) and extracted with ethyl acetate (2×50 mL). The combined organic extracts were washed with water (50 mL), with brine (50 mL) dried over Na₂SO₄ and concentrated under reduced and purified prep. chromatography to afford the desired compound (100 mg, 37.03%) as off white solid. LCMS: (M+1)⁺442.3; ¹H NMR (400 MHz, DMSO-d₆): δ 10.76 (s, 1H), 9.57 (br. s., 1H), 8.55-8.62 (m, 2H), 8.11 (d, J=5.70 Hz, 1H), 7.55 (br. s., 1H), 7.36 (s, 2H), 7.20-7.26 (m, 1H), 6.47 (d, J=6.14 Hz, 2H), 2.82-2.92 (m, 6H), 0.65-0.73 (m, 3H), 0.56-0.63 (m, 1H)

### Example-31: Synthesis of 4-((5-(5-chloro-2-fluorophenyl)-2-(dimethylamino)-6-hydroxypyridin-3-yl)amino)-N-(1-hydroxy-2-methylpropan-2-yl)nicotinamide (Compound 1.31)

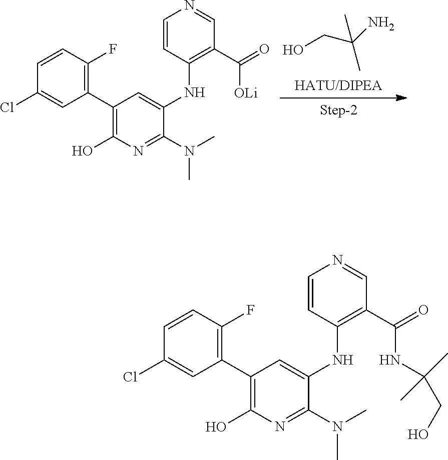

Steps 1 is same as in Example 30.

### Step-2: Synthesis of 4-((5-(5-chloro-2-fluorophenyl)-2-(dimethylamino)-6-hydroxypyridin-3-yl)amino)-N-(1-hydroxy-2-methylpropan-2-yl)nicotinamide

To a stirred solution of lithium 4-((5-(5-chloro-2-fluorophenyl)-2-(dimethylamino)-6-hydroxypyridin-3-yl)amino)nicotinate (0.250 g, 0.613 mmol, 1.0 eq) in DMF (3 mL) was added HATU (0.349 g, 0.919 mmol, 1.5 eq) at rt. The resulting mixture was stirred at rt for 5 minutes, followed by addition of 2-amino-2-methylpropan-1-ol (0.09 ml, 0.919 mmol, 1.5 eq) and DIPEA (0.3 mL, 1.8 mmol, 3.0 eq) and stirred at rt for 30 min. Reaction mixture was diluted with water (50 mL) and extracted with ethyl acetate (2×50 mL). The combined organic extracts were washed with water (50 mL), with brine (50 mL) dried over Na₂SO₄ and concentrated under reduced and purified prep. chromatography to afford the compound (100 mg, 34.6%) as off white solid. LCMS: (M+1)⁺474.2; ¹H NMR (400 MHz, DMSO-d₆): δ 8.12 (d, J=5.26 Hz, 2H), 7.36 (br. s., 4H), 6.47 (br. s., 2H), 4.86 (br. s., 1H), 3.52 (d, J=5.70 Hz, 3H), 2.89 (s, 7H), 1.32 (s, 6H)

### Example-32: Synthesis of 4-((5-(5-chloro-2-fluorophenyl)-2-(dimethylamino)-6-hydroxypyridin-3-yl)amino)nicotinamide (Compound 1.32)

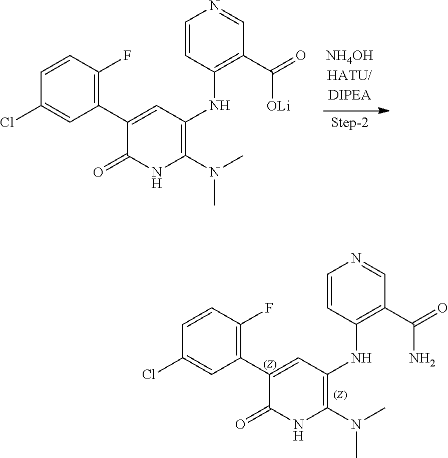

Steps 1 is same as in Example 30.

### Step-2: Synthesis of 4-((5-(5-chloro-2-fluorophenyl)-2-(dimethylamino)-6-hydroxypyridin-3-yl)amino)nicotinamide

To a stirred solution of lithium 4-((5-(5-chloro-2-fluorophenyl)-2-(dimethylamino)-6-hydroxypyridin-3-yl)amino)nicotinate (0.250 g, 0.613 mmol, 1.0 eq) in DMF (3 mL) was added HATU (0.280 g, 0.735 mmol, 1.5 eq) at rt. The resulting mixture was stirred at rt for 5 minutes, followed by addition of ammonium hydroxide (30%, 0.12 ml, 0.981 mmol, 2.0 eq) and DIPEA (0.25 mL, 1.4 mmol, 3.0 eq) and stirred at rt for 30 min. Reaction mixture was diluted with water (50 mL) and extracted with ethyl acetate (2×50 mL). The combined organic extracts were washed with water (50 mL), with brine (50 mL) dried over Na₂SO₄ and concentrated under reduced and purified prep. chromatography to afford the desired compound (23 mg, 11.73%) as off white solid. LCMS: (M+1)⁺402.2; ¹H NMR (400 MHz, DMSO-d₆): δ 10.77 (d, J=14.47 Hz, 1H), 9.87 (br. s., 1H), 8.69 (s, 1H), 8.10-8.16 (m, 2H), 7.54 (br. s., 2H), 7.36 (br. s., 2H), 7.22-7.27 (m, 1H), 6.45 (d, J=5.70 Hz, 1H), 2.86-2.93 (m, 6H)

### Example-33: Synthesis of 3-(5-chloro-2-fluorophenyl)-6-(dimethylamino)-5-((7-(trifluoromethyl)quinolin-4-yl)amino)pyridin-2(1H)-one (Compound 1.33)

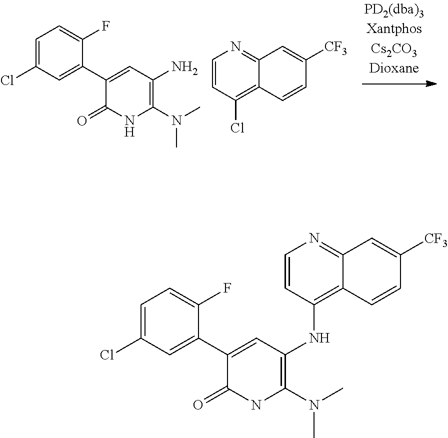

To a stirred solution of compound 5-amino-3-(5-chloro-2-fluorophenyl)-6-(dimethylamino)pyridin-2(1H)-one (0.20 g, 0.709 mmol, 1.0 eq) and 4-chloro-7-(trifluoromethyl)quinoline (0.197 g, 0.851 mmol, 1.2 eq) in dioxane (10 mL) was added Cs₂CO₃ (0.925 g, 2.836 mmol, 4.0 eq) at rt. The resulting mixture was purged with nitrogen for 10 min followed by addition of Pd₂(dba)₃ (0.078 g, 0.085 mmol, 0.12 eq) and xantphos (0.074 g, 0.127 mmol, 0.15 eq), again purged with nitrogen for 10 min. The reaction mixture was heated at 100° C. for overnight. The progress of reaction was monitored by LCMS. The reaction mixture was diluted with water (50 mL), extracted with EtOAc (2×50 mL). The combined organic layers were washed with water (50 mL), with brine (50 mL), dried over Na₂SO₄, concentrated and purified by combi flash [silica gel 100-200 mesh; elution 0-50% EtOAc in Hexane] to afford the desired compound (28 mg, 8.28%) as yellow solid. LCMS: (M+1)⁺477.1; ¹H NMR (400 MHz, DMSO-d₆): δ10.84 (brs, 1H), 8.90 (brs., 1H), 8.64 (d, J=8.80 Hz, 1H), 8.50 (d, J=5.38 Hz, 1H), 8.15 (s, 1H), 7.77 (d, J=8.80 Hz, 1H), 7.56 (d, J=3.91 Hz, 1H), 7.42 (s, 1H), 7.31-7.38 (m, 1H), 7.20-7.29 (m, 1H), 6.29 (d, J=5.38 Hz, 1H), 2.92 (s, 6H).

### Example-34: Synthesis of 4-((5-(5-chloro-2-fluorophenyl)-2-(dimethylamino)-6-oxo-1,6-dihydropyridin-3-yl)amino)nicotinonitrile (Compound 1.34)

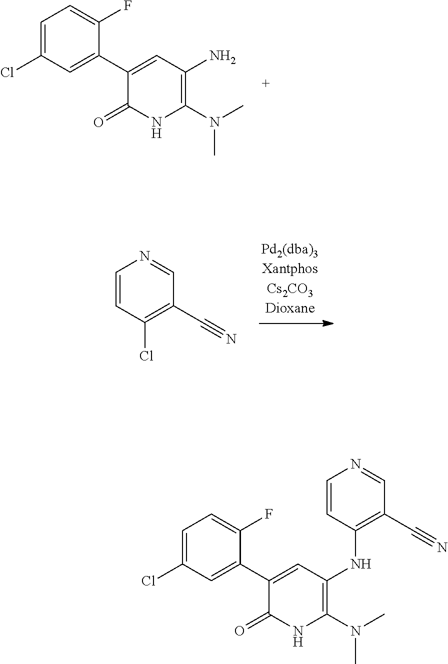 [SMILES: CN(C)c1[nH]c(=O)c(-c2cc(Cl)ccc2F)cc1N ; N#Cc1cnccc1Cl ; CN(C)c1[nH]c(=O)c(-c2cc(Cl)ccc2F)cc1Nc1ccncc1C#N]

To a stirred solution of compound 5-amino-3-(5-chloro-2-fluorophenyl)-6-(dimethylamino)pyridin-2(1H)-one (0.15 g, 0.532 mmol, 1.0 eq) and 4-chloronicotinonitrile (0.081 g, 0.585 mmol, 1.1 eq) in dioxane (5.0 mL) was added Cs₂CO₃ (0.694 g, 2.128 mmol, 4.0 eq) at rt. The resulting mixture was purged with nitrogen for 10 min followed by addition of Pd₂(dba)₃ (0.058 g, 0.064 mmol, 0.12 eq) and xantphos (0.046 g, 0.0.079 mmol, 0.15 eq), again purged with nitrogen for 10 min. The reaction mixture was heated at 120° C. for overnight. The progress of reaction was monitored by LCMS. The reaction mixture was diluted with EtOAc (100 mL), washed with water (50.0 mL) dried over Na₂SO₄, concentrated and purified by combi flash chromatography [silica gel 100-200 mesh; elution 0-100% EtOAc in Hexane] to afford the desired compound (24 mg, 11.76%) as orange solid. LCMS: (M+1)⁺: 384.1; ¹H NMR (400 MHz, DMSO-d₆): δ10.86 (brs, 1H) 8.75 (brs, 1H) 8.49 (s, 1H) 8.20 (d, J=6.14 Hz, 1H) 7.52 (brs, 1H) 7.32-7.41 (m, 2H) 7.14-7.27 (m, 1H) 6.40 (brs, 1H) 2.92 (s, 6H).

### Example-35: Synthesis of 3-(5-chloro-2-fluorophenyl)-5-((3-(4,5-dihydro-1H-imidazol-2-yl)pyridin-4-yl)amino)-6-(dimethylamino)pyridin-2(1H)-one (Compound 1.35)

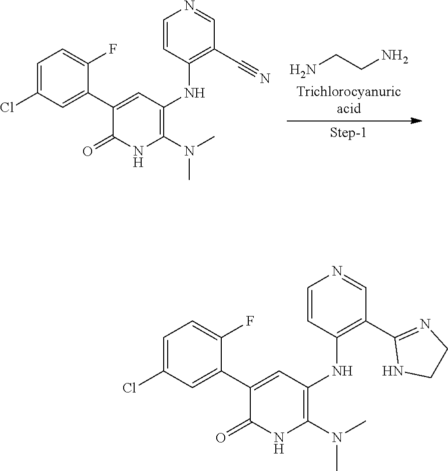

4-((5-(5-chloro-2-fluorophenyl)-2-(dimethylamino)-6-oxo-1,6-dihydropyridin-3-yl)amino)nicotinonitrile (350 mg, 0.911 mmol, 1.0 eq), ethane-1,2-diamine (0.25 mL, 3.647 mmol, 4.0 eq) and trichlorocyanuric acid (21 mg, 0.0911 mmol, 0.1 eq) were heated at 110° C. for 150 min. The progress of reaction was monitored by LCMS. The reaction mixture was diluted with EtOAc (100 mL), washed with water (50.0 mL) dried over Na₂SO₄, concentrated and purified by reverse phase purification to afford the desired compound (40 mg, 10.25%) as yellow solid. LCMS: (M+1)⁺: 427.3; ¹H NMR (400 MHz, DMSO-d₆): δ 11.14 (brs, 1H) 10.61 (s, 1H) 8.56 (s, 1H) 8.08 (d, J=5.70 Hz, 1H) 7.54 (d, J=3.51 Hz, 1H) 7.15-7.40 (m, 4H) 6.45 (d, J=5.70 Hz, 1H) 3.90 (t, J=9.21 Hz, 2H) 3.39 (d, J=9.65 Hz, 2H) 2.87 (s, 6H).

### Example-36 and Example-37: Synthesis of 5-((1,6-naphthyridin-4-yl)amino)-3-(5-chloro-2-fluorophenyl)-6-(methylthio)pyridin-2(1H)-one (Compound 1.36) and 5-((1,6-naphthyridin-4-yl)amino)-3-(5-chloro-2-fluorophenyl)-6-(dimethylamino)pyridin-2(1H)-one (Compound 1.37)

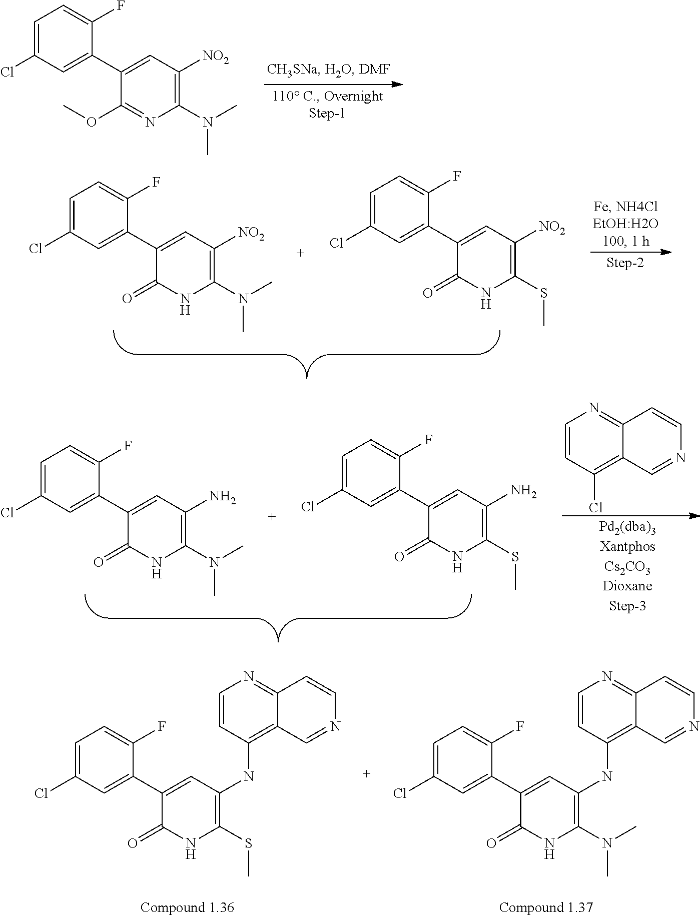

### Step-1: Synthesis of 3-(5-chloro-2-fluorophenyl)-6-(dimethylamino)-5-nitropyridin-2(1H)-one and 3-(5-chloro-2-fluorophenyl)-6-(methylthio)-5-nitropyridin-2(1H)-one

To a stirred solution of 5-(5-chloro-2-fluorophenyl)-6-methoxy-N,N-dimethyl-3-nitropyridin-2-amine (8.7 g, 26.710 mmol, 1.0 eq) in DMF (50 mL) was added 21% solution of CH₃SNa (7.48 g, 106.840 mmol, 4.0 eq) in water (35.6 mL) at rt. The reaction mixture was stirred at 110° C. for overnight. The progress of reaction was monitored by LCMS. The reaction mixture was acidified with 1N HCl (5.0 mL). The reaction mixture was poured in ice cold water (500 mL), filtered and the residue was washed with water (500 mL), dried under vacuum to afford the mixture of compounds (6.70 g, 80.72%) as yellow solid. LCMS: (M+1)⁺: 312.2 and 315.0

### Step-2: Synthesis of 5-amino-3-(5-chloro-2-fluorophenyl)-6-(dimethylamino)pyridin-2(1H)-one and 5-amino-3-(5-chloro-2-fluorophenyl)-6-(methylthio)pyridin-2(1H)-one

To a stirred solution of 3-(5-chloro-2-fluorophenyl)-6-(dimethylamino)-5-nitropyridin-2(1H)-one and 3-(5-chloro-2-fluorophenyl)-6-(methylthio)-5-nitropyridin-2(1H)-one (2.0 g, 6.410 mmol, 1.0 eq) in EtOH (30 mL) and H₂O (30 ml) was added Fe (2.87 g, 51.33 mmol, 8.0 eq) and a solution of NH₄Cl (3.43 g, 64.1 mmol, 10.0 eq) at rt. The resulting mixture was heated at 100° C. for 60 min. The progress of reaction was monitored by LCMS. The reaction mixture was filtered through celite the residue was washed with EtOH (50 mL) the filtrate was concentrated and the residue was dissolved in EtOAc (200 mL), washed with water (2×100 mL), dried over Na₂SO₄, and concentrated to afford the desired compound as brown solid. LCMS: (M+1)⁺282.2 and 285.0

### Step-3: Synthesis of 5-((1,6-naphthyridin-4-yl)amino)-3-(5-chloro-2-fluorophenyl)-6-(methylthio)pyridin-2(1H)-one dihydrochloride and 5-((1,6-naphthyridin-4-yl)amino)-3-(5-chloro-2-fluorophenyl)-6-(dimethylamino)pyridin-2(1H)

To a stirred solution of compound 5-amino-3-(5-chloro-2-fluorophenyl)-6-(dimethylamino)pyridin-2(1H)-one and 5-amino-3-(5-chloro-2-fluorophenyl)-6-(methylthio)pyridin-2(1H)-one (0.200 g, 0.709 mmol, 1.0 eq) and 4-chloro-1,6-naphthyridine (0.128 g, 0.780 mmol, 1.1 eq) in dioxane (30.0 mL) was added Cs₂CO₃ (0.925 g, 2.836 mmol, 4.0 eq) at rt. The resulting mixture was purged with nitrogen for 10 min followed by addition of Pd₂(dba)₃ (0.0.078 g, 0.085 mmol, 0.12 eq) and xantphos (0.061 g, 0.106 mmol, 0.15 eq), again purged with nitrogen for 10 min. The reaction mixture was heated at 120° C. for overnight. The progress of reaction was monitored by LCMS. The reaction mixture was diluted with water (50 mL), extracted with EtOAc (2×100 mL). The combined organic layers were washed with water (50 mL), with brine (50 mL), dried over Na₂SO₄, concentrated and purified by combi flash chromatography [silica gel 100-200 mesh: elution 0-10% MeOH in DCM] to afford the desired compounds A (40 mg, 13.74%) as yellow solid and Be (100 mg, 29.41%) as orange solid. LCMS A: (M+1)⁺: 410.3; ¹H NMR (400 MHz, DMSO-d₆) A: δ 10.88 (brs, 1H) 9.75 (s, 1H) 9.16 (brs, 1H) 8.61 (s, 1H) 8.50 (d, J=5.38 Hz, 1H) 7.65 (d, J=5.87 Hz, 1H) 7.58 (brs, 1H) 7.43 (s, 1H) 7.34 (brs, 1H) 7.14-7.29 (m, 1H) 6.26 (d, J=5.38 Hz, 1H) 2.93 (s, 6H). LCMS B: (M+1)⁺: 413.2; ¹H NMR (400 MHz, DMSO-d₆) B: δ 11.83 (brs, 1H) 11.33 (brs, 1H) 10.06 (brs, 1H) 8.96 (d, J=5.70 Hz, 1H) 8.63 (d, J=7.02 Hz, 1H) 7.93 (d, J=5.70 Hz, 1H) 7.79 (s, 1H) 7.55-7.62 (m, 1H) 7.48-7.55 (m, 1H) 7.36 (t, J=9.21 Hz, 1H) 6.62 (brs, 1H) 2.55 (s, 3H).

### Example-38: Synthesis of 4-((5-(5-chloro-2-fluorophenyl)-2-(dimethylamino)-6-oxo-1,6-dihydro pyridin-3-yl)amino)-N-methylnicotinamide (Compound 1.38)

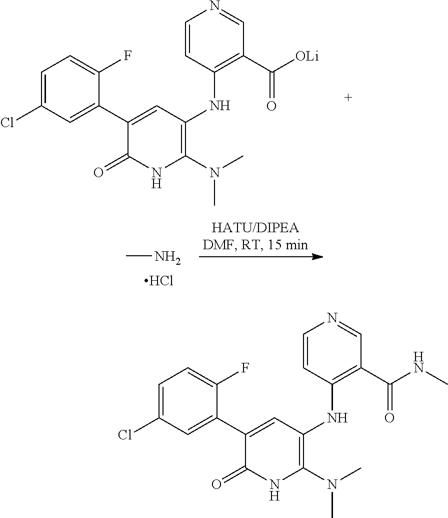

To a stirred solution of lithium 4-((5-(5-chloro-2-fluorophenyl)-2-(dimethylamino)-6-oxo-1,6-dihydropyridin-3-yl)amino)nicotinate (0.150 g, 0.366 mmol, 1.0 eq) in DMF (2 mL) was added HATU (0.208 g, 0.550 mmol, 1.5 eq) at rt. The resulting mixture was stirred at rt for 5 minutes, followed by addition of methanamine hydrochloride (0.049 g, 0.732 mmol, 2.0 eq) and DIPEA (0.19 mL, 1.098 mmol, 3.0 eq) and stirred at rt for 15 min. Reaction mixture was diluted with water (50 mL) and extracted with ethyl acetate (2×50 mL). The combined organic extracts were washed with water (50 mL), with brine (50 mL) dried over Na₂SO₄ and concentrated under reduced and purified combi flash chromatography [silica gel 100-200 mesh; elution 0-5% MeOH in DCM to afford the desired compound (20 mg, 13.15%) as orange solid. LCMS: (M+1)⁺416.3; ¹H NMR (400 MHz, DMSO-d₆): δ 10.80 (brs, 1H) 9.69 (brs, 1H) 8.65 (d, J=4.38 Hz, 1H) 8.61 (s, 2H) 8.13 (d, J=5.70 Hz, 1H) 7.55 (d, J=3.95 Hz, 1H) 7.36 (s, 2H) 7.20-7.30 (m, 2H) 6.49 (d, J=6.14 Hz, 2H) 2.89 (s, 6H) 2.79 (d, J=4.39 Hz, 3H).

### Example-39: Synthesis of 4-((5-(5-chloro-2-fluorophenyl)-2-(methylthio)-6-oxo-1,6-dihydropyridin-3-yl)amino)-N-methylnicotinamide (Compound 1.39)

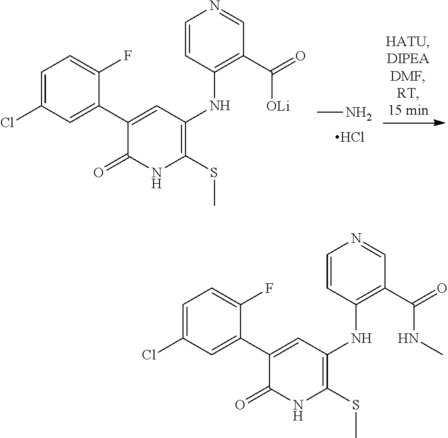

To a stirred solution of lithium 4-((5-(5-chloro-2-fluorophenyl)-2-(methylthio)-6-oxo-1,6-dihydropyridin-3-yl)amino)nicotinate (0.100 g, 0.242 mmol, 1.0 eq) in DMF (2 mL) was added HATU (0.138 g, 0.364 mmol, 1.5 eq) at rt. The resulting mixture was stirred at rt for 5 minutes, followed by addition of methanamine hydrochloride (0.25 g, 0.364 mmol, 2.0 eq) and DIPEA (0.12 mL, 0.726 mmol, 3.0 eq) and stirred at rt for 15 min. Reaction mixture was diluted with water (50 mL) and extracted with ethyl acetate (2×50 mL). The combined organic extracts were washed with water (50 mL), with brine (50 mL) dried over Na₂SO₄ and concentrated under reduced and purified combi flash chromatography [silica gel 100-200 mesh; elution 0-5% MeOH in DCM to afford the desired compound (10 mg, 9.90%) as white solid. LCMS: (M+1)⁺419.3, ¹H NMR (400 MHz, DMSO-d₆): δ 11.44 (brs, 1H) 10.06 (brs, 1H) 8.76 (brs, 1H) 8.68 (s, 1H) 8.16 (d, J=6.36 Hz, 1H) 7.57 (brs, 2H) 7.45 (brs, 1H) 7.32 (t, J=9.29 Hz, 1H) 6.56 (d, J=5.87 Hz, 1H) 2.81 (d, J=4.40 Hz, 3H).

### Example-40: Synthesis of N-(4-((5-(5-chloro-2-fluorophenyl)-2-(dimethylamino)-6-oxo-1,6-dihydropyridin-3-yl)amino)pyridin-2-yl)acetamide (Compound 1.40)

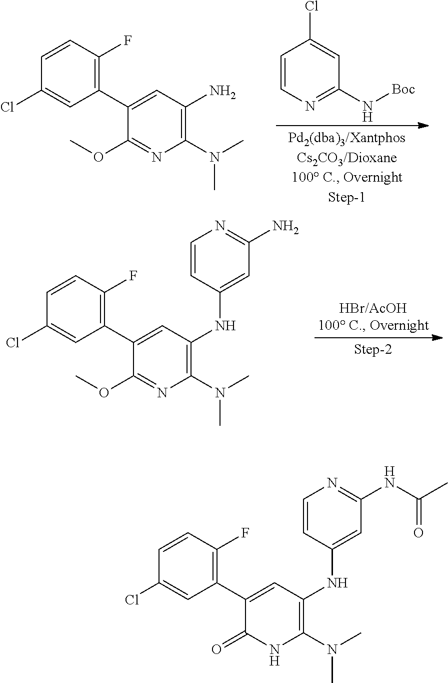

### Step-1: Synthesis of N3-(2-aminopyridin-4-yl)-5-(5-chloro-2-fluorophenyl)-6-methoxy-N2,N2-dimethylpyridine-2,3-diamine

To a stirred solution of compound 5-(5-chloro-2-fluorophenyl)-6-methoxy-N2,N2-dimethylpyridine-2,3-diamine (0.5 g, 1.69 mmol, 1.0 eq) and tert-butyl (4-chloropyridin-2-yl)carbamate (0.463 g, 2.02 mmol, 1.2 eq) in dioxane (20 mL) was added Cs₂CO₃ (2.20 g, 6.76 mmol, 4.0 eq) at rt. The resulting mixture was purged with nitrogen for 10 min followed by addition of Pd₂(dba)₃ (0.185 g, 0.20 mmol, 0.12 eq) and xantphos (0.146 g, 0.25 mmol, 0.15 eq), again purged with nitrogen for 10 min. The reaction mixture was heated at 130° C. for 48 h. The progress of reaction was monitored by LCMS. The reaction mixture was filtered through celite, the residue was washed with EtOAc (10 mL). The filtrate was concentrated and purified by combi flash chromatography [silica gel 100-200 mesh; elution 0-10 MeOH in DCM] to afford the desired compound (150 mg, 22.83%) as brown solid. LCMS: (M+1)⁺388.2.

### Step-2: Synthesis of N-(4-((5-(5-chloro-2-fluorophenyl)-2-(dimethylamino)-6-oxo-1,6-dihydropyridin-3-yl)amino)pyridin-2-yl)acetamide

N3-(2-aminopyridin-4-yl)-5-(5-chloro-2-fluorophenyl)-6-methoxy-N2,N2-dimethylpyridine-2,3-diamine (150 mg, 0.386 mmol, 1.0 eq) was dissolved in HBr in AcOH (33%) (2.0 mL) at rt. The reaction mixture was stirred at 100° C. for overnight. The progress of reaction was monitored by LCMS. The reaction mixture was basified with saturated aqueous solution of NaHCO₃(50 mL), extracted with EtOAc (2×50 mL). The combined organic layers were washed with water (50 mL), with brine (50 mL), dried over Na₂SO₄, concentrated and purified by reverse phase purification to afford the desired compound (5.20 mg, 3.25%) as light yellow solid. LCMS: (M+1)⁺416.3; ¹H NMR (400 MHz, DMSO-d₆): δ 13.15 (brs, 1H), 10.86 (brs, 1H), 7.85 (d, J=6.36 Hz, 1H), 7.52 (brs, 2H), 7.32-7.39 (m, 2H), 7.21-7.29 (m, 2H), 6.39 (s, 1H), 2.91 (s, 6H).

### Example-41: Synthesis of 3-(5-chloro-2-fluorophenyl)-6-(dimethylamino)-5-((3-fluoropyridin-4-yl)amino)pyridin-2 (1H)-one (Compound 1.41)

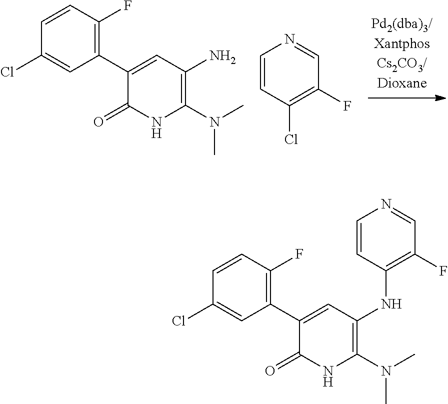

To a stirred solution of compound 5-amino-3-(5-chloro-2-fluorophenyl)-6-(dimethylamino)pyridin-2(1H)-one (0.5 g, 1.774 mmol, 1.0 eq) and 4-chloro-3-fluoropyridine (0.257 g, 1.952 mmol, 1.1 eq) in dioxane (20 mL) was added Cs₂CO₃ (2.31 g, 7.056 mmol, 4.0 eq) at rt. The resulting mixture was purged with nitrogen for 10 min followed by addition of Pd₂(dba)₃ (0.195 g, 0.212 mmol, 0.12 eq) and xantphos (0.153 g, 0.266 mmol, 0.15 eq), again purged with nitrogen for 10 min. The reaction mixture was heated at 130° C. for overnight. The progress of reaction was monitored by LCMS. The reaction mixture was filtered through celite, the residue was washed with EtOAc (100 mL). The filtrate was concentrated and purified by reverse phase purification to afford the desired compound (100 mg, 14.95%) as light yellow solid. LCMS: (M+1)⁺:377.3; ¹H NMR (400 MHz, DMSO-d₆): δ10.80 (brs, 1H), 8.25 (brs, 1H), 8.18 (brs, 1H), 8.10 (brs, 1H), 7.93 (brs, 1H), 7.53 (dd, J=2.45, 6.36 Hz, 1H), 7.11-7.39 (m, 3H), 6.26-6.36 (m, 1H), 2.91 (s, 6H).

### Example-42: Synthesis of 5-((1, 6-naphthyridin-4-yl)amino)-3-(5-chloro-2-fluorophenyl)-6-isopropylpyridin-2(1H)-one (Compound 1.42)

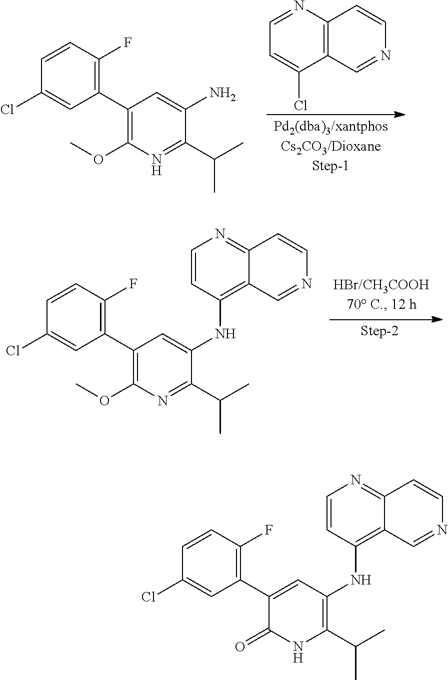

### Step-1: Synthesis of N-(5-(5-chloro-2-fluorophenyl)-2-isopropyl-6-methoxypyridin-3-yl)-1,6-naphthyridin-4-amine

To a stirred solution of compound 5-(5-chloro-2-fluorophenyl)-2-isopropyl-6-methoxypyridin-3-amine (0.200 g, 0.678 mmol, 1.0 eq) and 4-chloro-1,6-naphthyridine (0.128 g, 0.746 mmol, 1.1 eq) in dioxane (20 mL) was added Cs₂CO₃ (0.884 g, 2.712 mmol, 4.0 eq) at rt. The resulting mixture was purged with nitrogen for 10 min followed by addition of Pd₂(dba)₃ (0.075 g, 0.081 mmol, 0.12 eq) and xatphos (0.058 g, 0.101 mmol, 0.15 eq), again purged with nitrogen for 10 min. The reaction mixture was heated at 100° C. for overnight. The progress of reaction was monitored by LCMS. The reaction mixture was diluted with water (50 mL), extracted with EtOAc (2×100 mL). The combined organic layers were washed with water (50 mL), with brine (50 mL), dried over Na₂SO₄, concentrated and purified by combi flash chromatography [silica gel 100-200 mesh: elution 0-100% EtOAc in Hexane] to afford the desired compound (150 mg, 52.44%) as yellow solid. LCMS: (M+1)⁺423.2

### Step-2: Synthesis of 5-((1,6-naphthyridin-4-yl)amino)-3-(5-chloro-2-fluorophenyl)-6-isopropylpyridin-2(1H)-one

To a stirred solution of N-(5-(5-chloro-2-fluorophenyl)-2-isopropyl-6-methoxypyridin-3-yl)-1,6-naphthyridin-4-amine (0.150 g, 0.355 mmol, 1.0 eq) in HBr in acetic acid (33%) solution (3.0 mL) at rt. The reaction mixture was stirred at 100° C. for overnight. The progress of reaction was monitored by LCMS. The reaction mixture was basified with saturated aqueous solution of NaHCO₃ (100 mL), extracted with EtOAc (2×100 mL). The combined organic layers were washed with water (50 mL), with brine (50 mL), dried over Na₂SO₄, concentrated and purified by reverse phase purification to afford the desired compound (19 mg, 6.89%) as brown solid. LCMS: (M+1)⁺:409.2; ¹H NMR (400 MHz, DMSO-d₆): δ ppm 11.94 (brs, 1H) 9.75 (s, 1H) 9.09 (s, 1H) 8.63 (d, J=5.87 Hz, 1H) 8.52 (d, J=5.38 Hz, 1H) 7.62-7.75 (m, 2H) 7.57 (s, 1H) 7.36-7.47 (m, 1H) 7.30 (t, J=9.29 Hz, 1H) 6.35 (d, J=5.38 Hz, 1H) 3.03-3.14 (m, 1H) 1.22 (d, J=6.85 Hz, 6H).

### Example-43: Synthesis of 4-((5-(5-chloro-2-fluorophenyl)-2-isopropyl-6-oxo-1,6-dihydropyridin-3-yl)amino)nicotinamide (Compound 1.43)

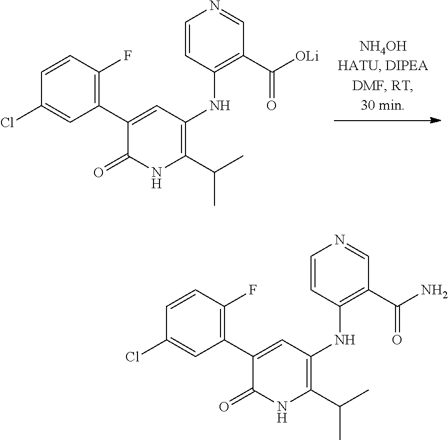

To a stirred solution of lithium 4-((5-(5-chloro-2-fluorophenyl)-2-isopropyl-6-oxo-1,6-dihydropyridin-3-yl)amino)nicotinate (0.140 g, 0.344 mmol, 1.0 eq) in DMF (2 mL) was added HATU (0.196 g, 0.516 mmol, 1.5 eq) at rt. The resulting mixture was stirred at rt for 5 minutes, followed by addition of NH₄OH (0.08 mL, 0.686 mmol, 2.0 eq) and DIPEA (0.18 mL, 1.03 mmol, 3.0 eq) and stirred at rt for 30 min. Reaction mixture was diluted with water (50 mL) and extracted with ethyl acetate (2×30 mL). The combined organic extracts were washed with water (50 mL), with brine (50 mL) dried over Na₂SO₄ and concentrated under reduced and purified reverse phase purification to afford the desired compound (27 mg, 19.70%) as white solid.

LCMS: (M+1)⁺401.3; ¹H NMR (400 MHz, DMSO-d₆): δ 11.80 (s, 1H) 9.80 (brs, 1H) 8.69 (s, 1H) 8.09-8.24 (m, 2H) 7.64 (d, J=2.45 Hz, 1H) 7.55 (brs, 1H) 7.34-7.49 (m, 2H) 7.28 (t, J=9.29 Hz, 1H) 6.42 (d, J=5.87 Hz, 1H) 2.99 (dd, J=14.43, 7.09 Hz, 1H) 1.06-1.32 (m, 6H).

### Example-44: Synthesis of 5-((2-aminopyridin-4-yl)amino)-3-(5-chloro-2-fluorophenyl)-6-isopropylpyridin-2(1H)-one (Compound 1.44)

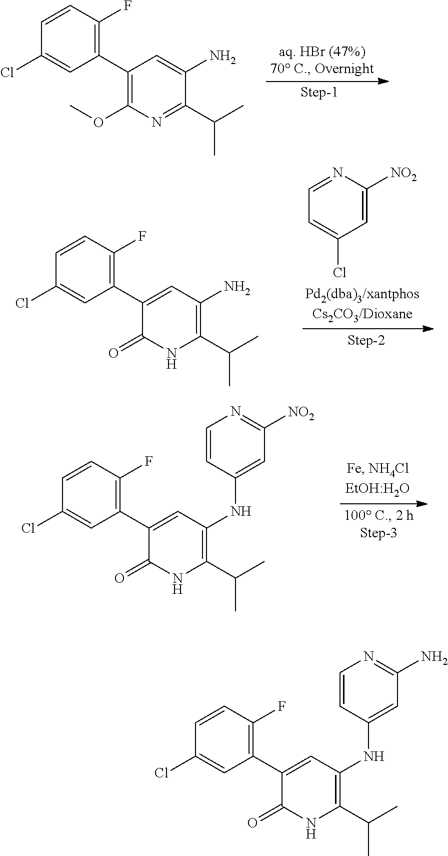

### Step-1: Synthesis of 5-amino-3-(5-chloro-2-fluorophenyl)-6-isopropylpyridin-2(1H)-one

To a stirred solution of 5-(5-chloro-2-fluorophenyl)-2-isopropyl-6-methoxypyridin-3-amine (1.50 g, 5.089 mmol, 1.0 eq) in HBr (47%) solution (10.0 mL) at rt. The reaction mixture was stirred at 70° C. for overnight. The progress of reaction was monitored by LCMS. The reaction mixture was basified with saturated aqueous solution of NaHCO₃ (100 mL), extracted with EtOAc (2×100 mL). The combined organic layers were washed with water (50 mL), with brine (50 mL), dried over Na₂SO₄, concentrated and purified by combi flash chromatography [silica gel 100-200 mesh; elution 0-30% EtOAc in Hexane] to afford the desired compound (1.20 g, 84.50%) as brown viscous. LCMS: (M+1)⁺281.0

### Step-2: Synthesis of 3-(5-chloro-2-fluorophenyl)-6-isopropyl-5-((2-nitropyridin-4-yl)amino)pyridin-2(1H)-one

To a stirred solution of 5-amino-3-(5-chloro-2-fluorophenyl)-6-isopropylpyridin-2(1H)-one (0.580 g, 2.06 mmol, 1.0 eq) and 4-chloro-2-nitropyridine (0.394 g, 2.479 mmol, 1.2 eq) in dioxane (20.0 mL) was added Cs₂CO₃ (2.02 g, 6.198 mmol, 3.0 eq) at rt. The resulting mixture was purged with nitrogen for 10 min followed by addition of Pd₂(dba)₃ (0.227 g, 0.247 mmol, 0.12 eq) and xatphos (0.180 g, 0.309 mmol, 0.15 eq), again purged with nitrogen for 10 min. The reaction mixture was heated at 120° C. for overnight. The progress of reaction was monitored by LCMS. The reaction mixture was diluted with water (50 mL), extracted with EtOAc (2×100 mL). The combined organic layers were washed with water (50 mL), with brine (50 mL), dried over Na₂SO₄, concentrated and purified by combi flash chromatography [silica gel 100-200 mesh: elution 0-50% EtOAc in Hexane] to afford the desired compound (400 mg, 48.07%) as brown solid. LCMS: (M+1)⁺403.1

### Step-3: Synthesis of 5-((2-aminopyridin-4-yl)amino)-3-(5-chloro-2-fluorophenyl)-6-isopropylpyridin-2(1H)-one

To a stirred solution of 3-(5-chloro-2-fluorophenyl)-6-isopropyl-5-((2-nitropyridin-4-yl)amino)pyridin-2(1H)-one (400 mg, 0.993 mmol, 1.0 eq) in EtOH (20.0 mL) and H₂O (20.0 mL) was added Fe (444 mg, 7.944 mmol, 8.0 eq) and NH₄Cl (531 mg, 9.930 mmol, 10.0 eq) at rt. The resulting mixture was heated at 100° C. for 60 min. The progress of reaction was monitored by LCMS. The reaction mixture was filtered through celite the residue was washed with EtOH (20 mL) the filtrate was concentrated and the residue was dissolved in EtOAc (50.0 mL), washed with water (50.0 mL), with brine (50.0 mL), dried over Na₂SO₄, and concentrated purified by reverse phase purification to afford the desired compound (21.0 mg, 5.67%) as brown solid. LCMS: (M+1)⁺373.3; ¹H NMR (400 MHz, DMSO-d₆): δ11.91-11.81 (m, 1H), 8.20 (d, J=12.5 Hz, 1H), 7.60 (dd, J=6.4, 2.8 Hz, 1H), 7.53 (d, J=6.4 Hz, 1H), 7.49-7.39 (m, 2H), 7.30 (t, J=9.2 Hz, 1H), 6.30 (s, 2H), 6.07-6.00 (m, 1H), 5.56 (s, 1H), 3.05 (hept, J=7.5, 7.1 Hz, 1H), 1.21 (d, J=7.1 Hz, 6H).

### Example-45: Synthesis of N-(4-((5-(5-chloro-2-fluorophenyl)-2-isopropyl-6-oxo-1, 6-dihydropyridin-3-yl)amino)pyridin-2-yl)cyclopropanecarboxamide (Compound 1.45)

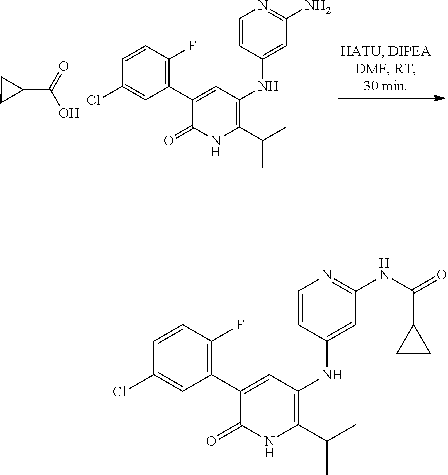

To a stirred solution of cyclopropanecarboxylic acid (0.030 g, 0.341 mmol, 1.0 eq) in DMF (3 mL) was added HATU (0.195 g, 0.511 mmol, 1.5 eq) at rt. The resulting mixture was stirred at rt for 5 minutes, followed by addition of 5-((2-aminopyridin-4-yl)amino)-3-(5-chloro-2-fluorophenyl)-6-isopropylpyridin-2(1H)-one (0.140 g, 0.375 mmol, 2.0 eq) and DIPEA (0.18 mL, 1.023 mmol, 3.0 eq) and stirred at rt for 30 min. Reaction mixture was diluted with water (50 mL) and extracted with ethyl acetate (2×30 mL). The combined organic extracts were washed with water (50 mL), with brine (50 mL) dried over Na₂SO₄ and concentrated under reduced and purified reverse phase purification to afford the desired compound (4.0 mg, 2.42%) as white solid. LCMS: (M+1)⁺441.2; ¹H NMR (400 MHz, DMSO-d₆): δ15.96 (s, 1H), 8.42 (d, J=10.2 Hz, 4H), 8.04 (s, 1H), 7.74 (s, 1H), 7.56 (dq, J=12.7, 4.4, 3.7 Hz, 2H), 7.48 (dd, J=6.4, 2.7 Hz, 1H), 7.44-7.35 (m, 1H), 6.02 (d, J=5.5 Hz, 1H), 5.72 (s, 1H), 5.58 (s, 2H), 2.08 (d, J=4.7 Hz, 1H), 1.73 (tt, J=8.1, 4.4 Hz, 1H), 1.30-1.18 (m, 1H), 1.15 (d, J=6.7 Hz, 6H), 0.94 (dq, J=7.1, 3.9 Hz, 2H), 0.76 (p, J=4.0 Hz, 4H).

### Example-46: Synthesis of N-(4-((5-(5-chloro-2-fluorophenyl)-2-isopropyl-6-oxo-1,6-dihydropyridin-3-yl)amino)pyridin-2-yl)-4,4,4-trifluorobutanamide (Compound 1.46)

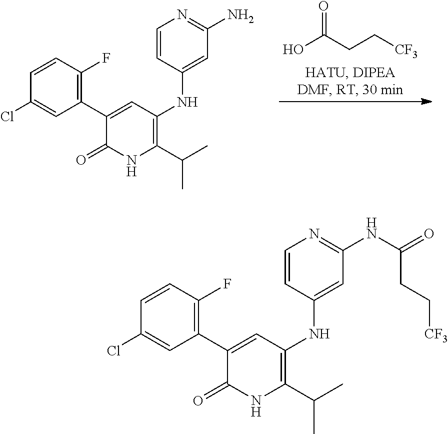 [SMILES: CC(C)c1[nH]c(=O)c(-c2cc(Cl)ccc2F)cc1Nc1ccnc(N)c1 ; O=C(O)CCC(F)(F)F ; CC(C)c1[nH]c(=O)c(-c2cc(Cl)ccc2F)cc1Nc1ccnc(NC(=O)CCC(F)(F)F)c1]

To a stirred solution of 4,4,4-trifluorobutanoic acid (0.070 g, 0.482 mmol, 1.0 eq) in DMF (2 mL) was added HATU (0.230 g, 0.603 mmol, 1.5 eq) at rt. The resulting mixture was stirred at rt for 5 minutes, followed by addition of 5-((2-aminopyridin-4-yl)amino)-3-(5-chloro-2-fluorophenyl)-6-isopropylpyridin-2(1H)-one (0.150 g, 0.402 mmol, 2.0 eq) and DIPEA (0.21 mL, 1.206 mmol, 3.0 eq) and stirred at rt for 30 min. Reaction mixture was diluted with water (50 mL) and extracted with ethyl acetate (2×30 mL). The combined organic extracts were washed with water (50 mL), with brine (50 mL) dried over Na₂SO₄ and concentrated under reduced and purified reverse phase purification to afford the desired compound (10.0 mg, 5.02%) as white solid. LCMS: (M+1)⁺497.3; ¹H NMR (400 MHz, DMSO-d₆): δ 12.07 (brs, 1H) 8.36 (brs, 1H) 7.85 (d, J=5.70 Hz, 1H) 7.72 (brs, 1H) 7.60-7.66 (m, 1H) 7.47 (d, J=8.77 Hz, 1H) 7.33 (t, J=9.21 Hz, 1H) 6.53 (d, J=5.70 Hz, 1H) 6.36 (s, 1H) 6.02 (brs, 1H) 2.89-2.99 (m, 1H) 2.60-2.75 (m, 3H) 2.58 (brs, 1H) 1.21 (d, J=7.02 Hz, 3H) 0.96 (d, J=6.58 Hz, 3H). (t, J=7.24 Hz, 2H) 2.96 (t, J=7.24 Hz, 2H) 2.61-2.72 (m, 2H) 2.30 (s, 3H).

### Example-47: Synthesis of N-(4-((5-(5-chloro-2-fluorophenyl)-2-isopropyl-6-oxo-1,6-dihydropyridin-3-yl)amino)pyridin-2-yl)-2-(pyrrolidin-1-yl)acetamide (Compound 1.47)

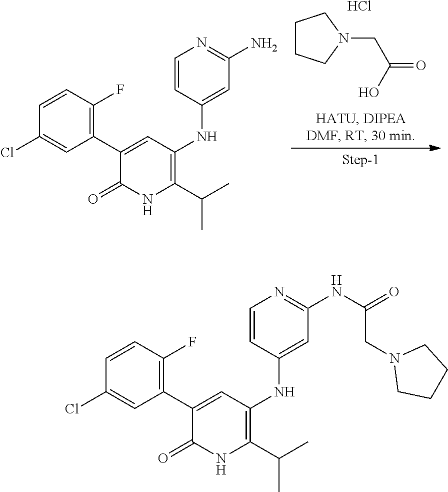

To a stirred solution of 2-(pyrrolidin-1-yl)acetic acid hydrochloride (0.080 g, 0.482 mmol, 1.0 eq) in DMF (2 mL) was added HATU (0.230 g, 0.603 mmol, 1.5 eq) at rt. The resulting mixture was stirred at rt for 5 minutes, followed by addition of 5-((2-aminopyridin-4-yl)amino)-3-(5-chloro-2-fluorophenyl)-6-isopropylpyridin-2(1H)-one (0.150 g, 0.402 mmol, 2.0 eq) and DIPEA (0.21 mL, 1.206 mmol, 3.0 eq) and stirred at rt for 30 min. Reaction mixture was diluted with water (50 mL) and extracted with ethyl acetate (2×30 mL). The combined organic extracts were washed with water (50 mL), with brine (50 mL) dried over Na₂SO₄ and concentrated under reduced and purified reverse phase purification to afford the desired compound (15.0 mg, 7.73%) as white solid.

LCMS: (M+1)⁺484.3; ¹H NMR (400 MHz, DMSO-d₆): δ 11.82 (brs, 1H) 9.62 (brs, 1H) 8.14 (s, 1H) 7.83 (d, J=5.70 Hz, 1H) 7.61 (dd, J=6.14, 2.63 Hz, 1H) 7.32-7.49 (m, 3H) 7.29 (t, J=9.43 Hz, 1H) 6.27 (brs, 1H) 3.03-3.13 (m, 1H) 2.67 (brs, 4H) 1.77 (brs, 4H) 1.20 (d, J=7.02 Hz, 6H).

### Example-48: Synthesis of N-(4-((5-(5-chloro-2-fluorophenyl)-2-isopropyl-6-oxo-1,6-dihydropyridin-3-yl)amino)pyridin-2-yl)acetamide (Compound 1.48)

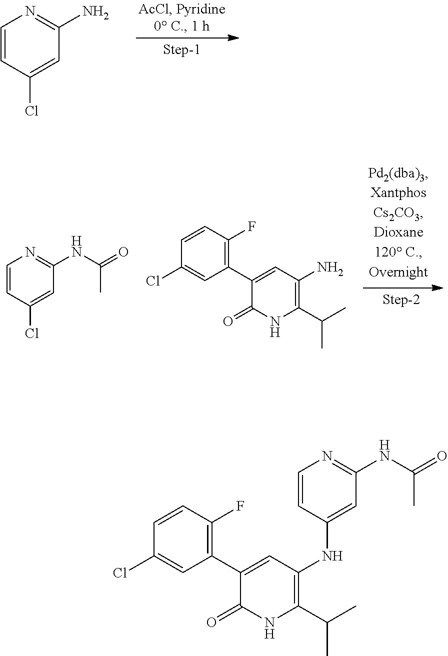

### Step-1: Synthesis of N-(4-chloropyridin-2-yl)acetamide

To a stirred solution of 4-chloropyridin-2-amine (2.0 g, 15.564 mmol, 1.0 eq) in pyridine (5.0 mL) was dropwise added AcCl (1.22 mL, 17.120 mmol, 1.10 eq) at 0° C. The resulting mixture was stirred at the same temperature for 1 h. The progress of reaction was monitored by LCMS. The reaction mixture was concentrated and purified by combi flash [silica gel 100-200 mesh; elution 0-30% EtOAc in Hexane] to afford the desired compound (1.80 g, 67.92%) as light yellow solid. LCMS: (M+1)⁺171.1

### Step-2: Synthesis of N-(4-((5-(5-chloro-2-fluorophenyl)-2-isopropyl-6-oxo-1,6-dihydropyridin-3-yl)amino)pyridin-2-yl)acetamide

To a stirred solution of compound 5-amino-3-(5-chloro-2-fluorophenyl)-6-isopropylpyridin-2(1H)-one (0.50 g, 1.781 mmol, 1.0 eq) and N-(4-chloropyridin-2-yl)acetamide (0.335 g, 1.959 mmol, 1.1 eq) in dioxane (30.0 mL) was added Cs₂CO₃ (1.75 g, 5.343 mmol, 3.0 eq) at rt. The resulting mixture was purged with nitrogen for 10 min followed by addition of Pd₂(dba)₃ (0.195 g, 0.214 mmol, 0.12 eq) and xatphos (0.154 g, 0.267 mmol, 0.15 eq), again purged with nitrogen for 10 min. The reaction mixture was heated at 120° C. for overnight. The progress of reaction was monitored by LCMS. The reaction mixture was filtered through celite filtrate was concentrated and purified by combi flash [silica gel 100-200 mesh; elution 0-10% MeOH in DCM] to afford the desired compound (35 mg, 4.73%) as white solid. LCMS: (M+1)⁺415.3; ¹H NMR (400 MHz, DMSO-d₆): δ ppm 13.21 (brs, 1H) 12.02 (brs, 1H) 11.59 (brs, 1H) 9.76 (brs, 1H) 7.91 (d, J=7.02 Hz, 1H) 7.62 (dd, J=6.14, 2.63 Hz, 1H) 7.42-7.54 (m, 2H) 7.31 (t, J=9.21 Hz, 1H) 6.67 (brs, 1H) 2.88-3.07 (m, 1H) 2.15 (s, 3H) 1.22 (d, J=7.02 Hz, 6H).

### Example-49: Synthesis of N-(4-((5-(5-chloro-2-fluorophenyl)-2-isopropyl-6-oxo-1,6-dihydropyridin-3-yl)amino)pyridin-2-yl)pivalamide (Compound 1.49)

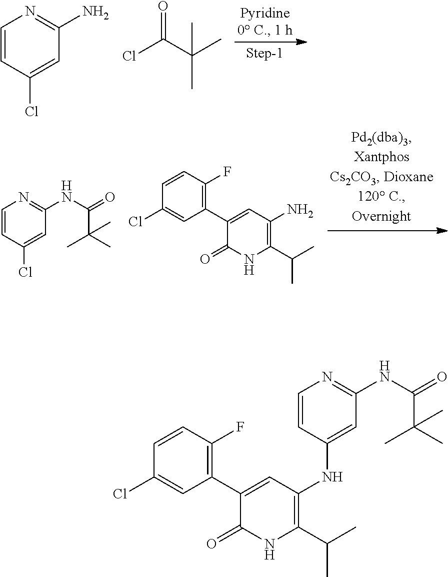

### Step-1: Synthesis N-(4-chloropyridin-2-yl)pivalamide

To a stirred solution of 4-chloropyridin-2-amine (2.6 g, 17.33 mmol, 1.0 eq) in pyridine (10.0 mL) was dropwise added PivCl (3.77 mL, 25.99 mmol, 1.50 eq) at 0° C. The resulting mixture was stirred at the rt for overnight. The progress of reaction was monitored by LCMS. The reaction mixture was concentrated and purified by combi flash [silica gel 100-200 mesh; elution 0-30% EtOAc in Hexane] to afford the desired compound of (1.80 g, 67.92%) as light yellow solid. LCMS: (M+1)⁺213.1

### Step-2: Synthesis of N-(4-((5-(5-chloro-2-fluorophenyl)-2-isopropyl-6-oxo-1,6-dihydropyridin-3-yl)amino)pyridin-2-yl)pivalamide

To a stirred solution of compound 5-amino-3-(5-chloro-2-fluorophenyl)-6-isopropylpyridin-2(1H)-one (0.30 g, 1.068 mmol, 1.0 eq) and N-(4-chloropyridin-2-yl)pivalamide (0.250 g, 1.175 mmol, 1.1 eq) in dioxane (30.0 mL) was added Cs₂CO₃ (1.05 g, 3.204 mmol, 3.0 eq) at rt. The resulting mixture was purged with nitrogen for 10 min followed by addition of Pd₂(dba)₃ (0.117 g, 0.128 mmol, 0.12 eq) and xatphos (0.092 g, 0.160 mmol, 0.15 eq), again purged with nitrogen for 10 min. The reaction mixture was heated at 120° C. for overnight. The progress of reaction was monitored by LCMS. The reaction mixture was filtered through celite filtrate was concentrated and purified by combi flash [silica gel 100-200 mesh; elution 0-10% MeOH in DCM] to afford the desired compound (60 mg, 12.37%) as white solid. LCMS: (M+1)⁺457.3; ¹H NMR (400 MHz, DMSO-d₆): δ 11.85 (brs, 1H) 9.57 (brs, 1H) 7.87 (d, J=5.70 Hz, 1H) 7.64 (dd, J=6.14, 2.63 Hz, 1H) 7.45 (brs, 2H) 7.30 (t, J=9.21 Hz, 2H) 6.38 (brs, 1H) 2.99-3.14 (m, 1H) 1.16-1.31 (m, 15H).

### Example-50: Synthesis of 5-((1H-pyrrolo[2,3-b]pyridin-4-yl)amino)-3-(5-chloro-2-fluorophenyl)-6-isopropylpyridin-2(1H)-one (Compound 1.50)

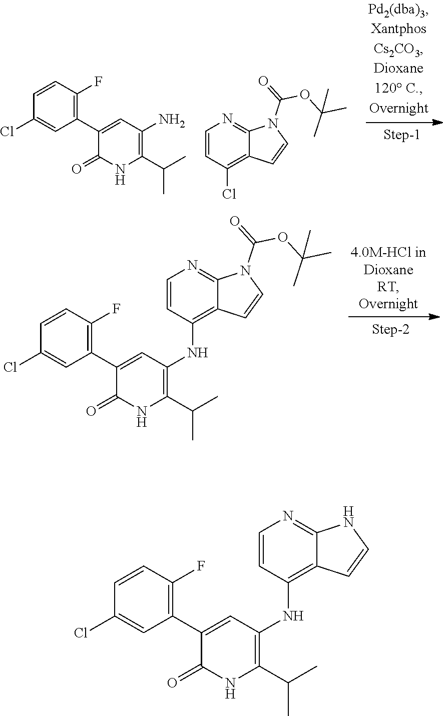

### Step-1: Synthesis of tert-butyl 4-((5-(5-chloro-2-fluorophenyl)-2-isopropyl-6-oxo-1,6-dihydropyridin-3-yl)amino)-1H-pyrrolo[2,3-b]pyridine-1-carboxylate

To a stirred solution of compound 5-amino-3-(5-chloro-2-fluorophenyl)-6-isopropylpyridin-2(1H)-one (0.167 g, 0.593 mmol, 1.0 eq) and tert-butyl 4-chloro-1H-pyrrolo[2,3-b]pyridine-1-carboxylate (0.180 g, 0.712 mmol, 1.2 eq) in dioxane (13.0 mL) was added Cs₂CO₃ (0.580 g, 1.779 mmol, 3.0 eq) at rt. The resulting mixture was purged with nitrogen for 10 min followed by addition of Pd₂(dba)₃ (0.065 g, 0.0716 mmol, 0.12 eq) and xatphos (0.052 g, 0.0889 mmol, 0.15 eq), again purged with nitrogen for 10 min. The reaction mixture was heated at 120° C. for overnight. The progress of reaction was monitored by LCMS. The reaction mixture was filtered through celite filtrate was concentrated and purified by combi flash [silica gel 100-200 mesh; elution 0-10% MeOH in DCM] to afford the desired compound (90 mg, 30.50%) as brown solid. LCMS: (M+1)⁺497.3

### Step-2: Synthesis 5-((1H-pyrrolo[2,3-b]pyridin-4-yl)amino)-3-(5-chloro-2-fluorophenyl)-6-isopropylpyridin-2(1H)-one

tert-butyl 4-((5-(5-chloro-2-fluorophenyl)-2-isopropyl-6-oxo-1,6-dihydropyridin-3-yl)amino)-1H-pyrrolo[2,3-b]pyridine-1-carboxylate (0.090 g, 0.181 mmol, 1.0 eq) was dissolved in 4.0 M-HCl in dioxane (2.0 mL) at rt. The resulting mixture was stirred at the rt for overnight. The progress of reaction was monitored by LCMS. The reaction mixture was neutralized with saturated solution of NaHCO₃ (250 mL), extracted with EtOAc (2×100 mL), the combined organic layers were washed with water (50.0 mL), with brine (50.0 mL), dried over Na₂SO₄ concentrated and purified by combi flash [silica gel 100-200 mesh; elution 0-10% MeOH in DCM] to afford the desired compound (8.0 mg, 11.11%) as off white solid. LCMS: (M+1)⁺397.2; ¹H NMR (400 MHz, DMSO-d₆): δ 11.87 (brs, 1H) 11.29 (brs, 1H) 8.05 (brs, 1H) 7.79 (d, J=5.70 Hz, 1H) 7.63 (dd, J=6.58, 2.63 Hz, 1H) 7.38-7.48 (m, 2H) 7.29 (t, J=9.43 Hz, 1H) 7.14 (brs, 1H) 6.49 (brs, 1H) 5.94 (d, J=5.26 Hz, 1H) 3.10-3.19 (m, 1H) 1.13-1.31 (m, 6H).

### Example-51: Synthesis of N-(4-((6′-fluoro-6-isopropyl-2-oxo-1,2-dihydro-[3,3′-bipyridin]-5-yl)amino)pyridin-2-yl)acetamide (Compound 1.51)

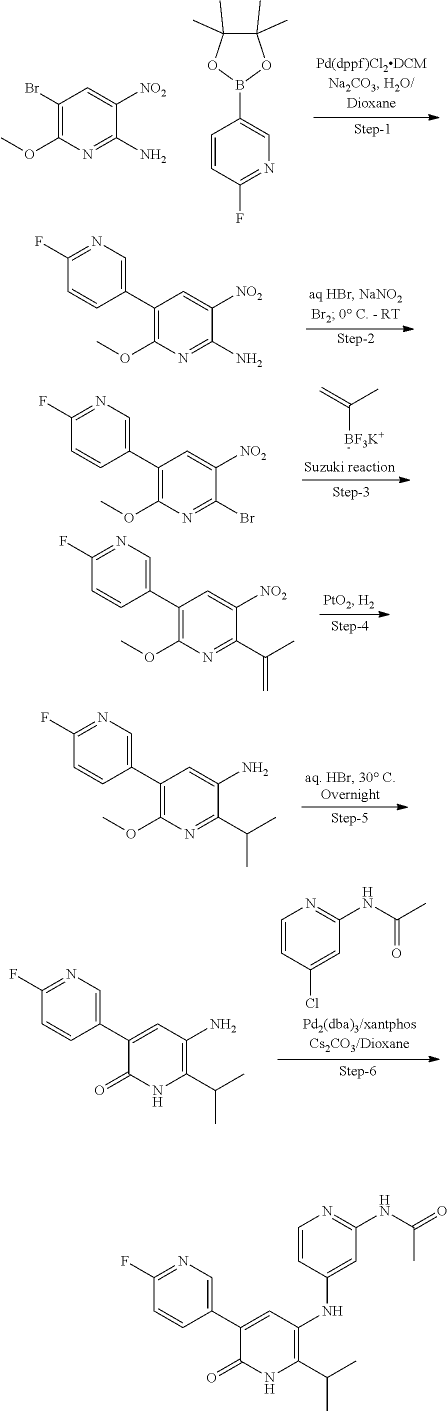

### Step-1: Synthesis of 6′-fluoro-2-methoxy-5-nitro-[3,3′-bipyridin]-6-amine

To a stirred solution of 5-bromo-6-methoxy-3-nitropyridin-2-amine (2.5 g, 10.09 mmol, 1.0 eq) and 2-fluoro-5-(4,4,5,5-tetramethyl-1,3,2-dioxaborolan-2-yl)pyridine (2.70 g, 12.104 mmol, 1.2 eq) in dioxane (40 mL) was added a 2M solution of Na₂CO₃ (2.14 g, 20.18 mmol, 2.0 eq) in water (10.09 mL) at rt. The resulting mixture was purged with nitrogen for 10 min followed by addition of Pd(dppf)Cl₂.DCM (0.083 g, 0.100 mmol, 0.01 eq), again purged with nitrogen for 10 min. The reaction mixture was heated at 120° C. for overnight. The progress of reaction was monitored by LCMS. The reaction mixture was diluted with water (50 mL), extracted with EtOAc (2×100 mL). The combined organic layers were washed with water (50 mL), with brine (50 mL), dried over Na₂SO₄, concentrated and purified by combi flash chromatography [silica gel 100-200 mesh: elution 0-10% EtOAc in Hexane] to afford the desired compound (1.30 g, 48.87%) as yellow solid. LCMS: (M+1)⁺265.1

### Step-2: Synthesis of 6-bromo-6′-fluoro-2-methoxy-5-nitro-3,3′-bipyridine

To a stirred solution of 6′-fluoro-2-methoxy-5-nitro-[3,3′-bipyridin]-6-amine (1.30 g, 4.920 mmol, 1.0 eq) in aqueous HBr (47%) (4.2 mL, 24.60 mmol, 5.0 eq) was added a solution of NaNO₂ (1.19 g, 17.22 mmol, 3.5 eq) in water (2.0 mL) at 0° C., followed by addition of bromine (0.84 mL, 17.22 mmol, 3.5 eq). The resulting mixture was stirred for overnight at the room temperature. The progress of reaction was monitored by LCMS. The reaction mixture was basified with saturated solution of 6N-NaOH solution (100 mL), extracted with EtOAc (2×100 mL). The combined organic layers were washed with water (50 mL), with brine (50 mL), dried over Na₂SO₄, concentrated and purified by combi flash chromatography [silica gel 100-200 mesh: elution 0-10% EtOAc in Hexane] to afford the desired compound (1.20 g, 74.53%) as brown solid. LCMS: (M+1)⁺328.0

### Step-3: Synthesis of 6′-fluoro-2-methoxy-5-nitro-6-(prop-1-en-2-yl)-3,3′-bipyridine

To a stirred solution of 6-bromo-6′-fluoro-2-methoxy-5-nitro-3,3′-bipyridine (1.20 g, 3.657 mmol, 1.0 eq) and potassium trifluoro(prop-1-en-2-yl)borate (0.70 g, 4.754 mmol, 1.3 eq) in dioxane (20.0 mL) was added a 2M solution of Na₂CO₃ (0.77 g, 7.314 mmol, 2.0 eq) in water (3.65 mL) at rt. The resulting mixture was purged with nitrogen for 10 min followed by addition of Pd(dppf)Cl₂.DCM (0.030 g, 0.036 mmol, 0.01 eq), again purged with nitrogen for 10 min. The reaction mixture was heated at 120° C. for overnight. The progress of reaction was monitored by LCMS. The reaction mixture was diluted with water (50 mL), extracted with EtOAc (2×100 mL). The combined organic layers were washed with water (50 mL), with brine (50 mL), dried over Na₂SO₄, concentrated and purified by combi flash chromatography [silica gel 100-200 mesh: elution 0-10% EtOAc in Hexane] to afford the desired compound (0.450 g, 42.85%) as light yellow solid. LCMS: (M+1)⁺290.1

### Step-4: Synthesis of 6′-fluoro-6-isopropyl-2-methoxy-[3,3′-bipyridin]-5-amine

To a stirred solution of 6′-fluoro-2-methoxy-5-nitro-6-(prop-1-en-2-yl)-3,3′-bipyridine (0.40 g, 1.382 mmol, 1.0 eq) in methanol (20 mL) was added PtO₂ (100 mg) at rt. The reaction mixture was purged with hydrogen for 1 h. The progress of reaction was monitored by LCMS. The reaction mixture was filtered and the residue was washed with methanol (50 mL), filtrate was concentrated to afford the desired compound (0.310 g, 85.87%) as brown solid. LCMS: (M+1)⁺262.2

### Step-5: Synthesis of 5-amino-6′-fluoro-6-isopropyl-[3,3′-bipyridin]-2(1H)-one

To a stirred solution of 6′-fluoro-6-isopropyl-2-methoxy-[3,3′-bipyridin]-5-amine (0.14 g, 0.535 mmol, 1.0 eq) in acetic acid (2.0 mL) was added 48% aqueous HBr solution (0.27 mL, 1.607 mmol, 3.0 eq) at rt. The reaction mixture was stirred at 50° C. for 4 h. The progress of reaction was monitored by LCMS. The reaction mixture was basified with saturated aqueous solution of NaHCO₃ (50 mL), extracted with EtOAc (2×50 mL). The combined organic layers were washed with water (50 mL), with brine (50 mL), dried over Na₂SO₄, and concentrated to afford the desired compound (100 mg, 75.75%) as brown solid. LCMS: (M+1)⁺248.2

### Step-6: Synthesis of N-(4-((6′-fluoro-6-isopropyl-2-oxo-1,2-dihydro-[3,3′-bipyridin]-5-yl)amino)pyridin-2-yl)acetamide

To a stirred solution of compound 5-amino-6′-fluoro-6-isopropyl-[3,3′-bipyridin]-2(1H)-one (0.10 g, 0.404 mmol, 1.0 eq) and N-(4-chloropyridin-2-yl)acetamide (0.075 g, 0.444 mmol, 1.1 eq) in dioxane (5.0 mL) was added Cs₂CO₃ (0.395 g, 1.212 5 mmol, 3.0 eq) at rt. The resulting mixture was purged with nitrogen for 10 min followed by addition of Pd₂(dba)₃ (0.044 g, 0.048 mmol, 0.12 eq) and xatphos (0.035 g, 0.06 mmol, 0.15 eq), again purged with nitrogen for 10 min. The reaction mixture was heated at 120° C. for overnight. The progress of reaction was monitored by LCMS. The reaction mixture was diluted with water (50 mL), extracted with EtOAc (2×100 mL). The combined organic layers were washed with water (50 mL), with brine (50 mL), dried over Na₂SO₄, concentrated and purified by reverse phase purification to afford the desired compound (5.0 mg, 3.24%) as off white solid. LCMS: (M+1)⁺382.3; ¹H NMR (400 MHz, DMSO-d₆): δ11.87 (brs, 1H) 10.21 (brs, 1H) 8.58 (s, 1H) 8.37 (t, J=8.33 Hz, 1H) 8.14 (brs, 1H) 7.83 (d, J=5.70 Hz, 1H) 7.63 (s, 1H) 7.50 (brs, 1H) 7.38 (d, J=6.58 Hz, 1H) 7.20 (dd, J=8.55, 2.85 Hz, 1H) 6.29 (brs, 1H) 2.99-3.13 (m, 1H) 1.07-1.32 (m, 6H).

### Example-52: Synthesis of 5-((1H-pyrrolo[2,3-b]pyridin-4-yl)amino)-6′-fluoro-6-isopropyl-[3,3′-bipyridin]-2(1H)-one (Compound 1.52)

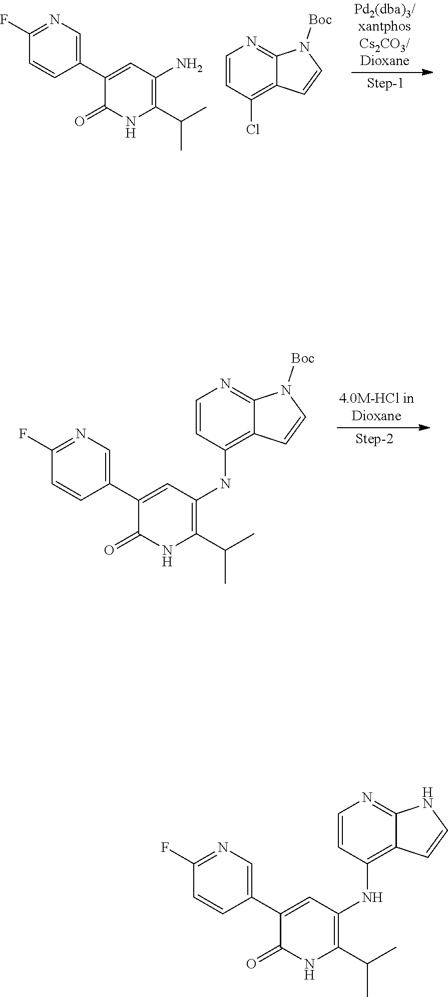

### Step-1: Synthesis of tert-butyl 4-((6′-fluoro-6-isopropyl-2-oxo-1,2-dihydro-[3,3′-bipyridin]-5-yl)amino)-1H-pyrrolo[2,3-b]pyridine-1-carboxylate

To a stirred solution of compound 5-amino-6′-fluoro-6-isopropyl-[3,3′-bipyridin]-2(1H)-one (0.100 g, 0.405 mmol, 1.0 eq) and tert-butyl 4-chloro-1H-pyrrolo[2,3-b]pyridine-1-carboxylate (0.112 g, 0.444 mmol, 1.2 eq) in dioxane (5.0 mL) was added Cs₂CO₃ (0.394 g, 1.215 mmol, 3.0 eq) at rt. The resulting mixture was purged with nitrogen for 10 min followed by addition of Pd₂(dba)₃ (0.045 g, 0.049 mmol, 0.12 eq) and xatphos (0.035 g, 0.060 mmol, 0.15 eq), again purged with nitrogen for 10 min. The reaction mixture was heated at 120° C. for overnight. The progress of reaction was monitored by LCMS. The reaction mixture was filtered through celite filtrate was concentrated and purified by combi flash [silica gel 100-200 mesh; elution 0-10% MeOH in DCM] to afford the desired compound (100 mg, 53.47%) as brown solid. LCMS: (M+1)⁺464.2

### Step-2: Synthesis 5-((1H-pyrrolo[2,3-b]pyridin-4-yl)amino)-6′-fluoro-6-isopropyl-[3,3′-bipyridin]-2(1H)-one

tert-butyl 4-((6′-fluoro-6-isopropyl-2-oxo-1,2-dihydro-[3,3′-bipyridin]-5-yl)amino)-1H-pyrrolo[2,3-b]pyridine-1-carboxylate (0.100 g, 0.216 mmol, 1.0 eq) was dissolved in 4.0 M-HCl in dioxane (2.0 mL) at rt. The resulting mixture was stirred at the rt for 2 h. The progress of reaction was monitored by LCMS. The reaction mixture was neutralized with saturated solution of NaHCO₃ (30 mL), extracted with EtOAc (2×50 mL), the combined organic layers were washed with water (50.0 mL), with brine (50.0 mL), dried over Na₂SO₄ concentrated and purified by reverse phase purification to afford the desired compound (6.0 mg, 7.69%) as yellow solid. LCMS: (M+1)⁺364.2; ¹H NMR (400 MHz, DMSO-d₆): δ 13.99 (brs, 1H) 12.21 (brs, 1H) 12.06 (brs, 1H) 9.60 (brs, 1H) 8.59 (brs, 1H) 8.39 (brs, 1H) 7.95 (d, J=6.58 Hz, 1H) 7.80 (s, 1H) 7.34 (brs, 1H) 7.21 (d, J=6.14 Hz, 1H) 6.78 (brs, 1H) 6.35 (brs, 1H) 3.00-3.13 (m, 1H) 1.21 (d, J=7.02 Hz, 6H).

### Example-53: Synthesis of N-(4-((5-(2-(hydroxymethyl)phenyl)-2-isopropyl-6-oxo-1, 6-dihydropyridin-3-yl)amino)pyridin-2-yl)acetamide (Compound 1.53)

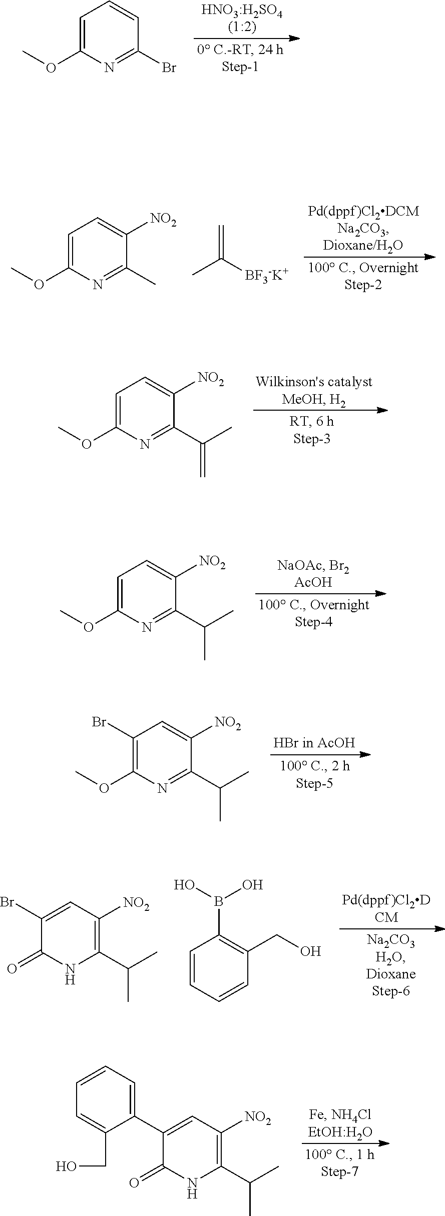

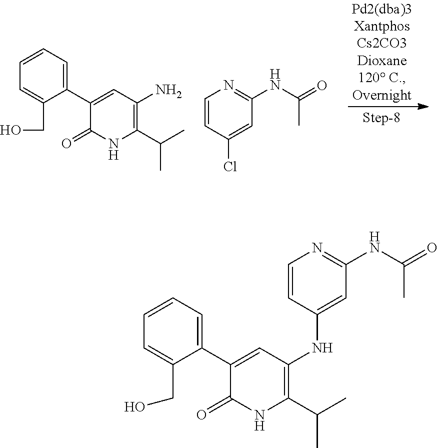

### Step-1: Synthesis of 2-bromo-6-methoxy-3-nitropyridine

2-bromo-6-methoxypyridine (10 mL, 81.374 mmol, 1.0 eq) was added to cold concentrated H₂SO₄ (40.0 mL followed by dropwise addition of HNO₃ (10.0 mL). The resulting mixture was stirred at rt for overnight. The progress of reaction was monitored by LCMS. The reaction mixture was poured into ice cold water (500.0 mL), and filtered to afford the desired compound (13.02 g, 68.67%) as light yellow solid. LCMS (M+1)⁺233.0; ¹H NMR (400 MHz, CDCl₃): δ 8.19 (d, J=9.21 Hz, 1H) 6.79 (d, J=8.77 Hz, 1H) 4.05 (s, 3H).

### Step-2: Synthesis 6-methoxy-3-nitro-2-(prop-1-en-2-yl)pyridine

To a stirred solution of 2-bromo-6-methoxy-3-nitropyridine (13.0 g, 55.789 mmol, 1.0 eq), and Potassium isopropenyltrifluoroborate (9.90 g, 66.947 mmol, 1.2 eq) in dioxane (100.0 mL) was added a 2 M solution of sodium carbonate (11.83 g, 111.578 mmol, 2.0 eq) in water (55.78 mL) at rt. The resulting mixture was purged with nitrogen for 1o minute followed by addition of Pd(dppf)Cl₂.DCM (0.92 g, 1.115 mmol, 0.02 eq), again purged with nitrogen for 10 minute. The resulting mixture was heated at 120° C. for overnight. The progress of reaction was monitored by LCMS. The reaction mixture was diluted with water (50 mL), extracted with EtOAc (2×100 mL). The combined organic layers were washed with water (50 mL), with brine (50 mL), dried over Na2SO4, concentrated and purified by combi flash chromatography [silica gel 100-200 mesh: elution 0-5% EtOAc in Hexane] to afford the desired (9.70 g, 89.56%) as light yellow liquid. LCMS: (M+1)⁺195.1; ¹H NMR (400 MHz, CDCl₃): δ 8.07 (d, J=9.21 Hz, 1H) 6.70 (d, J=8.77 Hz, 1H) 5.24-5.32 (m, 1H) 5.09 (s, 1H) 4.01 (s, 3H) 2.18 (s, 3H).

### Step-3: Synthesis of 2-isopropyl-6-methoxy-3-nitropyridine

To a stirred solution of 6-methoxy-3-nitro-2-(prop-1-en-2-yl)pyridine (8.50 g, 43.771 mmol, 1.0 eq) in MeOH (50 mL) was added wilkinson's catalyst (2.03 g, 2.188 mmol, 0.05 eq) at rt. The reaction mixture was purged with hydrogen at rt for 6 h. The progress of reaction was monitored by LCMS. The reaction mixture was filtered through celite and filtrate was concentrated and purified by combiflash chromatography [silica gel 100-200 mesh: elution 0-5% EtOAc in Hexane] to afford the desired compound (8.0 g, 93.24%) as light yellow liquid. LCMS: (M+1)⁺197.2; ¹H NMR (400 MHz, CDCl₃): δ 8.12 (d, J=8.77 Hz, 1H) 6.62 (d, J=8.77 Hz, 1H) 4.02 (s, 3H) 3.70-3.86 (m, 1H) 1.31 (d, J=6.58 Hz, 6H).

### Step-4: Synthesis of 3-bromo-6-isopropyl-2-methoxy-5-nitropyridine

To a stirred solution of 2-isopropyl-6-methoxy-3-nitropyridine (8.0 g, 40.77 mmol, 1.0 eq) in AcOH (50 mL) was added NaOAc (13.38 g, 163.08 mmol, 4.0 eq) at rt, followed by dropwise addition of bromine (4.07 mL, 81.54 mmol, 2.0 eq). The resulting mixture was heated at 100° C. for overnight. The progress of reaction was monitored by ¹HNMR. The reaction mixture was neutralized with saturated solution of NaHCO₃(500.0 mL) extracted with EtOAc (2×100 mL). The combined organic layers were washed with water (50 mL), with sodiumthiosulphate solution (200 mL), dried over Na₂SO₄, concentrated and purified by combi flash chromatography [silica gel 100-200 mesh: elution 0-5% EtOAc in Hexane] to afford the desired compound 3-bromo-6-isopropyl-2-methoxy-5-nitropyridine (7.50 g, 66.84%) as light yellow liquid. ¹H NMR (400 MHz, CDCl₃): δ 8.38 (s, 1H) 4.10 (s, 3H) 3.65-3.81 (m, 1H) 1.31 (d, J=7.02 Hz, 6H).

### Step-5: Synthesis of 3-bromo-6-isopropyl-5-nitropyridin-2(1H)-one

3-bromo-6-isopropyl-2-methoxy-5-nitropyridine (7.50 g, 27.262 mmol, 1.0 eq) was dissolved in 33% HBr in acetic acid (50.0 mL) at rt. The reaction mixture was stirred at 80° C. for 2 h. The progress of reaction was monitored by LCMS. The reaction mixture was basified with saturated aqueous solution of NaHCO₃(500 mL), extracted with EtOAc (2×100 mL). The combined organic layers were washed with water (50 mL), with brine (50 mL), dried over Na₂SO₄, and concentrated to afford the desired compound (7.02 g, 98.73%) as yellow solid. LCMS: (M+1)⁺261.1; ¹H NMR (400 MHz, DMSO-d₆): δ 12.54 (brs, 1H) 8.43 (s, 1H) 3.75 (dt, J=14.03, 7.02 Hz, 1H) 1.29 (d, J=7.02 Hz, 6H).

### Step-6: Synthesis 3-(2-(hydroxymethyl)phenyl)-6-isopropyl-5-nitropyridin-2(1H)-one

To a stirred solution of 3-bromo-6-isopropyl-5-nitropyridin-2(1H)-one (0.50 g, 1.915 mmol, 1.0 eq), and (2-(hydroxymethyl)phenyl)boronic acid (0.32 g, 2.106 mmol, 1.1 eq) in dioxane (20.0 mL) was added a 2 M solution of sodium carbonate (0.405 g, 3.83 mmol, 2.0 eq) in water (1.9 mL) at rt. The resulting mixture was purged with nitrogen for 10 minute followed by addition of Pd(dppf)Cl₂.DCM (0.031 g, 0.038 mmol, 0.02 eq), again purged with nitrogen for 10 minute. The resulting mixture was heated at 120° C. for overnight. The progress of reaction was monitored by LCMS. The reaction mixture was diluted with EtOAc (20 mL), washed with water (20 mL), with brine (20 mL), dried over Na₂SO₄, concentrated and purified by combi flash chromatography [silica gel 100-200 mesh: elution 0-30% EtOAc in Hexane] to afford the desired compound (0.34 g, 61.59%) as brown solid. LCMS: (M+1)⁺271.2

### Step-7: Synthesis of 5-amino-3-(2-(hydroxymethyl)phenyl)-6-isopropylpyridin-2(1H)-one

To a stirred solution of 3-(2-(hydroxymethyl)phenyl)-6-isopropyl-5-nitropyridin-2(1H)-one (0.33 g, 1.144 mmol, 1.0 eq) in EtOH (20.0 mL) and water (20.0 mL) was added Fe (0.511 g, 9.157 mmol, 8.0 eq) and a solution of NH₄Cl (0.612 g, 11.44 mmol, 10.0 eq) at rt. The resulting mixture was heated at 100° C. for 60 min. The progress of reaction was monitored by LCMS. The reaction mixture was filtered through celite the residue was washed with EtOH (50 mL) the filtrate was concentrated and the residue was dissolved in EtOAc (200 mL), washed with water (2×50 mL), dried over Na₂SO₄, and concentrated to afford the desired (0.020 g, 67.79%) as brown solid. LCMS: (M+1)⁺259.2

### Step-8: Synthesis of N-(4-((5-(2-(hydroxymethyl)phenyl)-2-isopropyl-6-oxo-1,6-dihydropyridin-3-yl)amino)pyridin-2-yl)acetamide

To a stirred solution of compound 5-amino-3-(2-(hydroxymethyl)phenyl)-6-isopropylpyridin-2(1H)-one (0.20 g, 0.774 mmol, 1.0 eq) and N-(4-chloropyridin-2-yl)acetamide (0.158 g, 0.929 mmol, 1.2 eq) in dioxane (20 mL) was added Cs₂CO₃ (0.755 g, 2.322 mmol, 3.0 eq) at rt. The resulting mixture was purged with nitrogen for 10 min followed by addition of Pd₂(dba)₃ (0.085 g, 0.093 mmol, 0.12 eq) and xantphos (0.067 g, 0.116 mmol, 0.15 eq), again purged with nitrogen for 10 min. The reaction mixture was heated at 120° C. for overnight. The progress of reaction was monitored by LCMS. The reaction mixture was diluted with EtOAc (100 mL), washed with water (50 mL), with brine (50 mL), dried over Na₂SO₄, concentrated and purified by combi flash chromatography [silica gel 100-200 mesh: elution 0-10% MeOH in DCM] to afford the desired compound (12.2 mg, 4.02%) as off white solid. LCMS: (M+1)⁺393.4; ¹H NMR (400 MHz, DMSO-d₆): δ ppm 11.74 (brs, 1H) 10.20 (brs, 1H) 8.14 (brs, 1H) 7.82 (d, J=5.70 Hz, 1H) 7.50 (d, J=7.89 Hz, 1H) 7.34 (t, J=7.24 Hz, 1H) 7.25-7.31 (m, 1H) 7.11-7.22 (m, 3H) 6.27 (brs, 1H) 5.08 (t, J=5.48 Hz, 1H) 4.40 (d, J=5.70 Hz, 2H) 3.03-3.11 (m, 1H) 2.03 (s, 3H) 1.16-1.26 (m, 6H).

### Example-54: Synthesis of N-(4-((5-(3-cyanophenyl)-2-isopropyl-6-oxo-1,6-dihydropyridin-3-yl)amino)pyridin-2-yl)acetamide (Compound 1.54)

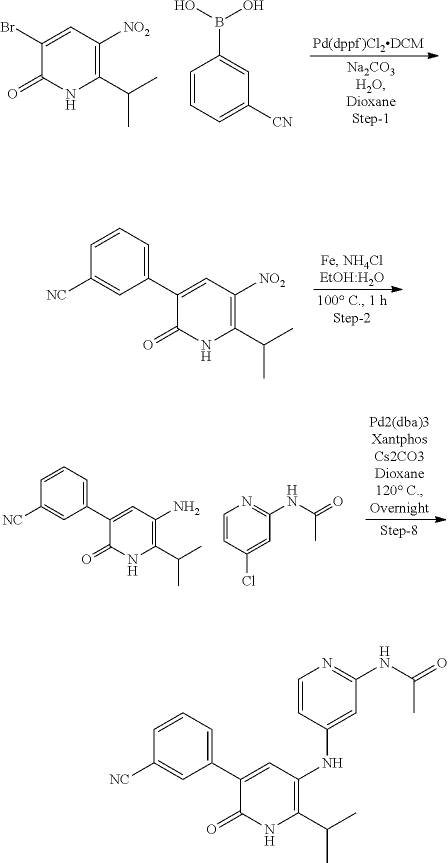

### Step-1: Synthesis 3-(6-isopropyl-5-nitro-2-oxo-1,2-dihydropyridin-3-yl)benzonitrile

To a stirred solution of 3-bromo-6-isopropyl-5-nitropyridin-2(1H)-one (0.50 g, 1.915 mmol, 1.0 eq), and (3-cyanophenyl)boronic acid (0.31 g, 2.106 mmol, 1.1 eq) in dioxane (20.0 mL) was added a 2 M solution of sodium carbonate (0.405 g, 3.83 mmol, 2.0 eq) in water (1.9 mL) at rt. The resulting mixture was purged with nitrogen for 10 minute followed by addition of Pd(dppf)Cl₂.DCM (0.031 g, 0.038 mmol, 0.02 eq), again purged with nitrogen for 10 minute. The resulting mixture was heated at 120° C. for overnight. The progress of reaction was monitored by LCMS. The reaction mixture was diluted with EtOAc (20 mL), washed with water (20 mL), with brine (20 mL), dried over Na₂SO₄, concentrated and purified by combi flash chromatography [silica gel 100-200 mesh: elution 0-30% EtOAc in Hexane] to afford the desired (0.36 g, 66.42%) as brown solid. LCMS: (M+1)⁺: 284.2

### Step-2: Synthesis of 3-(5-amino-6-isopropyl-2-oxo-1,2-dihydropyridin-3-yl)benzonitrile

To a stirred solution of 3-(6-isopropyl-5-nitro-2-oxo-1,2-dihydropyridin-3-yl)benzonitrile (350 g, 1.235 mmol, 1.0 eq) in EtOH (15.0 mL) and water (15.0 mL) was added Fe (0.551 g, 9.883 mmol, 8.0 eq) and a solution of NH₄Cl (0.660 g, 12.35 mmol, 10.0 eq) at rt. The resulting mixture was heated at 100° C. for 60 min. The progress of reaction was monitored by LCMS. The reaction mixture was filtered through celite the residue was washed with EtOH (50 mL) the filtrate was concentrated and the residue was dissolved in EtOAc (100 mL), washed with water (50 mL), dried over Na₂SO₄, and concentrated to afford the desired compound (0.30 g, 96.15%) as brown solid. LCMS: (M+1)⁺254.0

### Step-3: Synthesis of N-(4-((5-(3-cyanophenyl)-2-isopropyl-6-oxo-1,6-dihydropyridin-3-yl)amino)pyridin-2-yl)acetamide

To a stirred solution of compound 3-(5-amino-6-isopropyl-2-oxo-1,2-dihydropyridin-3-yl)benzonitrile (0.32 g, 1.263 mmol, 1.0 eq) and N-(4-chloropyridin-2-yl)acetamide (0.258 g, 1.515 mmol, 1.2 eq) in dioxane (30 mL) was added Cs₂CO₃ (1.238 g, 3.789 mmol, 3.0 eq) at rt. The resulting mixture was purged with nitrogen for 10 min followed by addition of Pd₂(dba)₃ (0.138 g, 0.1515 mmol, 0.12 eq) and xantphos (0.11 g, 0.189 mmol, 0.15 eq), again purged with nitrogen for 10 min. The reaction mixture was heated at 120° C. for overnight. The progress of reaction was monitored by LCMS. The reaction mixture was diluted with EtOAc (100 mL), washed with water (50 mL), with brine (50 mL), dried over Na₂SO₄, concentrated and purified by combi flash chromatography [silica gel 100-200 mesh: elution 0-10% MeOH in DCM] to afford the desired compound (130.0 mg, 26.53%) as cream colored solid. LCMS: (M+1)⁺388.4; ¹H NMR (400 MHz, DMSO-d₆): δ 11.85 (brs, 1H) 10.16 (brs, 1H) 8.25 (s, 1H) 8.12 (d, J=7.89 Hz, 2H) 7.82 (d, J=5.70 Hz, 1H) 7.58 (t, J=7.67 Hz, 1H) 7.35 (br. s., 1H) 6.26 (br. s., 1H) 3.08 (dt, J=13.81, 7.13 Hz, 1H) 2.02 (s, 3H) 1.20 (d, J=7.02 Hz, 6H).

### Example-55: Synthesis of 3-(5-chloro-2-fluorophenyl)-5-((2-(cyclopropylamino)pyridin-4-yl)amino)-6-isopropylpyridin-2(1H)-one (Compound 1.55)

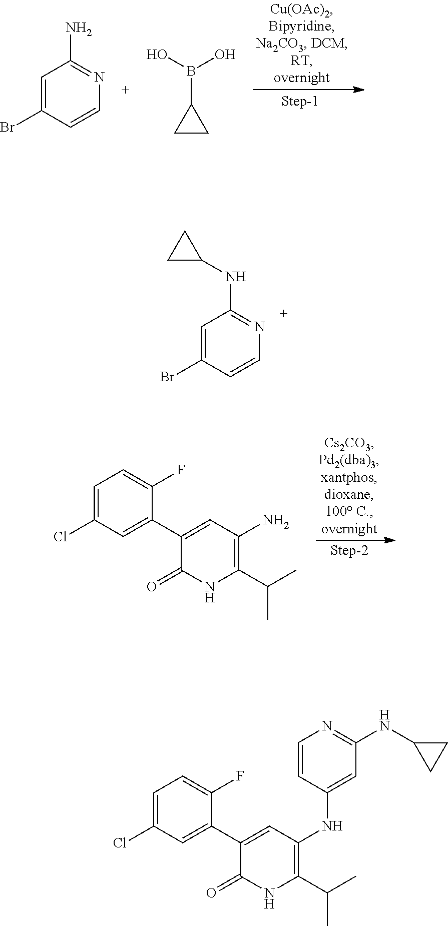 [SMILES: Nc1cc(Br)ccn1 ; OB(O)C1CC1 ; Brc1ccnc(NC2CC2)c1 ; CC(C)c1[nH]c(=O)c(-c2cc(Cl)ccc2F)cc1N ; CC(C)c1[nH]c(=O)c(-c2cc(Cl)ccc2F)cc1Nc1ccnc(NC2CC2)c1]

### Step-1: Synthesis of 4-bromo-N-cyclopropylpyridin-2-amine

To a solution of 4-bromopyridin-2-amine (500 mg, 2.9 mmol, 1 eq) in DCM (20 mL), was added cyclopropylboronic acid (375 mg, 4.36 mmol, 1.5 eq), sodium carbonate (922 mg, 8.7 mmol, 3 eq), bipyridine (680 mg, 4.36 mmol, 1.5 eq) and Cu(OAc)₂ (1056 mg, 5.8 mmol, 2 eq). The reaction mixture was then allowed to stir at rt for overnight. Progress of the reaction was monitored by TLC and LCMS. After completion of the reaction, diluted with ethyl acetate (150 mL) and washed with water (100 mL), brine solution (100 mL). Organic layer was dried over anhydrous sodium sulphate and concentrated under reduced pressure to obtain crude, which was purified by normal phase combi flash to obtain desired compound (200 mg, 32%) as an off white solid. LCMS: (M+1)⁺213

### Step-2: Synthesis of 3-(5-chloro-2-fluorophenyl)-5-((2-(cyclopropylamino)pyridin-4-yl)amino)-6-isopropylpyridin-2(3H)-one

To a solution of 6-(2-chloro-5-fluoropyrimidin-4-yl)-8-fluoro-4-isopropyl-3,4-dihydro-2H-benzo[b][1,4]oxazine (150 mg, 0.53 mmol, 1 eq) in dioxane (5 mL), was added 4-bromo-N-cyclopropylpyridin-2-amine (136 mg, 0.64 mmol, 1.2 eq) and cesium carbonate (346 mg, 1.06 mmol, 2 eq). The reaction mixture was degassed with nitrogen gas for 15 min., followed by the addition of Pd₂ (dba)₃ (24 mg, 0.026 mmol, 0.05 eq) and xantphos (31 mg, 0.053 mmol, 0.1 eq). The resultant reaction mixture was allowed to stir at 100° C. for overnight. Progress of the reaction was monitored by TLC and LCMS. After completion of the reaction, diluted with water (30 mL) and extracted with ethyl acetate (100 mL). Organic layer was washed with water (50 mL) and brine solution (50 mL). Organic layer was dried over anhydrous sodium sulphate and concentrated under reduced pressure to obtain crude, which was purified by reverse phase HPLC to obtain (10 mg, 5%) as a white color solid compound. LCMS: (M+1)⁺413.3; ¹H NMR (400 MHz, DMSO-d₆): δ 11.78 (brs, 1H), 7.81 (brs, 1H), 7.52-7.65 (m, 2H), 7.34-7.48 (m, 2H), 7.11-7.33 (m, 1H), 6.49 (brs, 1H), 5.93 (brs, 1H), 5.70 (brs., 1H), 3.11 (m, 1H), 2.37 (m, 1H), 1.21 (d, J=7.0 Hz, 6H), 0.59 (d, J=4.8 Hz, 2H), 0.35 ppm (brs, 2H).

### Example-56: Synthesis of N-(4-((6-isopropyl-2-oxo-6′-(trifluoromethyl)-, 2-dihydro-[3,3′-bipyridin]-5-yl)amino)pyridin-2-yl)acetamide (Compound 1.56)

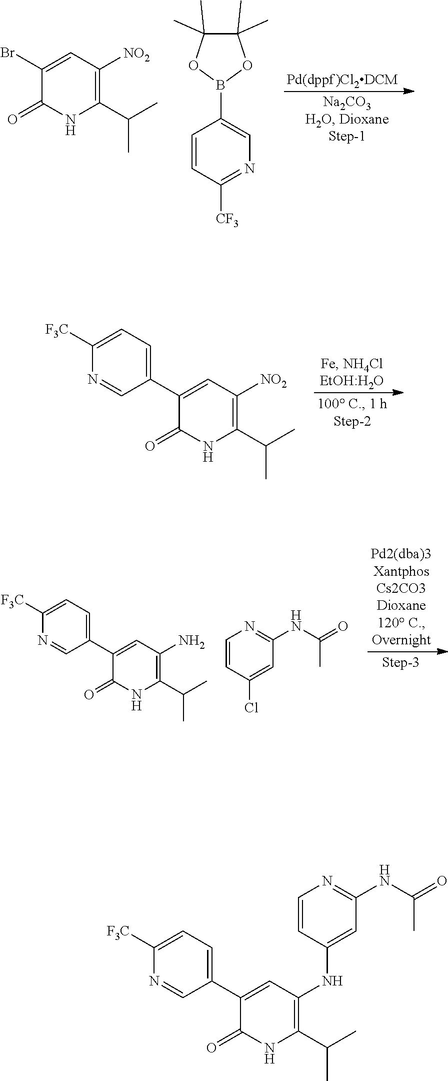

### Step-1: Synthesis 6-isopropyl-5-nitro-6′-(trifluoromethyl)-[3,3′-bipyridin]-2(1H)-one

To a stirred solution of 3-bromo-6-isopropyl-5-nitropyridin-2(1H)-one (0.435 g, 1.664 mmol, 1.0 eq), and 5-(4,4,5,5-tetramethyl-1,3,2-dioxaborolan-2-yl)-2-(trifluoromethyl)pyridine (0.50 g, 1.831 mmol, 1.1 eq) in dioxane (20.0 mL) was added a 2 M solution of sodium carbonate (0.352 g, 3.328 mmol, 2.0 eq) in water (1.6 mL) at rt. The resulting mixture was purged with nitrogen for 10 minute followed by addition of Pd(dppf)Cl₂.DCM (0.027 g, 0.033 mmol, 0.02 eq), again purged with nitrogen for 10 minute. The resulting mixture was heated at 120° C. for overnight. The progress of reaction was monitored by LCMS. The reaction mixture was diluted with EtOAc (20 mL), washed with water (20 mL), with brine (20 mL), dried over Na₂SO₄, concentrated and purified by combi flash chromatography [silica gel 100-200 mesh: elution 0-30% EtOAc in Hexane] to afford the desired compound (0.5 g, 91.74%) as light yellow solid. LCMS: (M+1)⁺328.1

### Step-2: Synthesis of 5-amino-6-isopropyl-6′-(trifluoromethyl)-[3,3′-bipyridin]-2(1H)-one

To a stirred solution of 6-isopropyl-5-nitro-6′-(trifluoromethyl)-[3,3′-bipyridin]-2(1H)-one (0.700 g, 2.138 mmol, 1.0 eq) in EtOH (25.0 mL) and water (25.0 mL) was added Fe (0.955 g, 17.111 mmol, 8.0 eq) and NH₄Cl (1.15 g, 21.38 mmol, 10.0 eq) at rt. The resulting mixture was heated at 100° C. for 60 min. The progress of reaction was monitored by LCMS. The reaction mixture was filtered through celite the residue was washed with EtOH (50 mL) the filtrate was concentrated and the residue was dissolved in EtOAc (100 mL), washed with water (20 mL), dried over Na₂SO₄, and concentrated to afford the desired (0.60 g, 96.30%) as brown solid. LCMS: (M+1)⁺298.1

### Step-3: Synthesis of N-(4-((6-isopropyl-2-oxo-6′-(trifluoromethyl)-1,2-dihydro-[3,3′-bipyridin]-5-yl)amino)pyridin-2-yl)acetamide

To a stirred solution of compound 5-amino-6-isopropyl-6′-(trifluoromethyl)-[3,3′-bipyridin]-2(1H)-one (0.50 g, 1.682 mmol, 1.0 eq) and N-(4-chloropyridin-2-yl)acetamide (0.315 g, 1.850 mmol, 1.2 eq) in dioxane (20 mL) was added Cs₂CO₃ (1.650 g, 5.046 mmol, 3.0 eq) at rt. The resulting mixture was purged with nitrogen for 10 min followed by addition of Pd₂(dba)₃ (0.185 g, 0.201 mmol, 0.12 eq) and xantphos (0.145 g, 0.252 mmol, 0.15 eq), again purged with nitrogen for 10 min. The reaction mixture was heated at 120° C. for overnight. The progress of reaction was monitored by LCMS. The reaction mixture was diluted with EtOAc (100 mL), washed with water (50 mL), with brine (50 mL), dried over Na₂SO₄, concentrated and purified by combi flash chromatography [silica gel 100-200 mesh: elution 0-10% MeOH in DCM] to afford the desired compound (180.0 mg, 24.89%) as off white solid. LCMS: (M+1)⁺423.4; ¹H NMR (400 MHz, DMSO-d₆): δ ppm 11.97 (brs, 1H) 10.17 (brs, 1H) 9.10 (s, 1H) 8.48 (d, J=9.65 Hz, 1H) 8.18 (brs, 1H) 7.91 (d, J=7.89 Hz, 1H) 7.71-7.89 (m, 3H) 7.35 (brs, 1H) 6.28 (brs, 1H) 3.03-3.12 (m, 1H) 1.98-2.05 (m, 3H) 1.21 (d, J=7.02 Hz, 6H).

### Example-57: Synthesis of 3-(5-chloro-2-fluorophenyl)-5-((5-fluoro-1H-pyrrolo[2,3-b]pyridin-4-yl)amino)-6-isopropylpyridin-2(1H)-one (Compound 1.57)

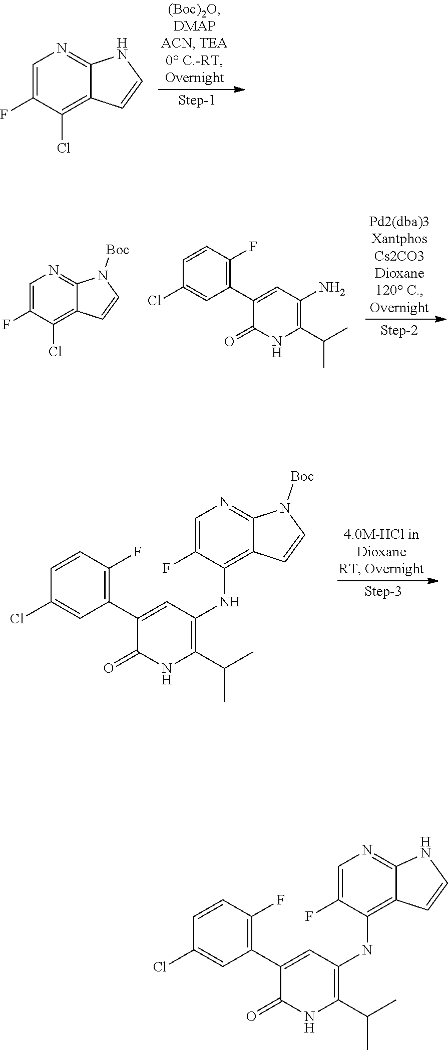

### Step-1: Synthesis ter-butyl 4-chloro-5-fluoro-1H-pyrrolo[2,3-b]pyridine-1-carboxylate

To a stirred solution of 4-chloro-5-fluoro-1H-pyrrolo[2,3-b]pyridine (1.0 g, 5.862 mmol, 1.0 eq), TEA (2.70 mL, 19.344 mmol, 3.0 eq), and DMAP (0.15 g, 1.172 mmol, 0.2 eq) in acetonitrile (20.0 mL) at rt. The resulting mixture was allowed to cool to 0° C. followed by addition (Boc)₂O (1.40 g, 6.448 mmol, 1.10 eq). The resulting mixture was stirred at the rt for overnight. The progress of reaction was monitored by LCMS. The reaction mixture was diluted with EtOAc 100 mL), washed with water (50.0 mL), with brine (50.0 mL), dried over Na₂SO₄ concentrated and purified by by combi flash [silica gel 100-200 mesh; elution 0-10% EtOAc in Hexane] to afford the desired compound (1.20 g, 75.94%) as light yellow liquid. LCMS: (M+1)⁺271.0

### Step-2: Synthesis of tert-butyl 4-((5-(5-chloro-2-fluorophenyl)-2-isopropyl-6-oxo-1,6-dihydropyridin-3-yl)amino)-5-fluoro-1H-pyrrolo[2,3-b]pyridine-1-carboxylate

To a stirred solution of compound 5-amino-3-(5-chloro-2-fluorophenyl)-6-isopropylpyridin-2(1H)-one (0.500 g, 1.781 mmol, 1.0 eq) and tert-butyl 4-chloro-5-fluoro-1H-pyrrolo[2,3-b]pyridine-1-carboxylate (0.530 g, 1.959 mmol, 1.2 eq) in dioxane (20.0 mL) was added Cs₂CO₃ (1.75 g, 5.343 mmol, 3.0 eq) at rt. The resulting mixture was purged with nitrogen for 10 min followed by addition of Pd₂(dba)₃ (0.195 g, 0.213 mmol, 0.12 eq) and xatphos (0.154 g, 0.267 mmol, 0.15 eq), again purged with nitrogen for 10 min. The reaction mixture was heated at 120° C. for overnight. The progress of reaction was monitored by LCMS. The reaction mixture was filtered through celite filtrate was concentrated and purified by combi flash [silica gel 100-200 mesh; elution 0-10% MeOH in DCM] to afford the desired compound tert-butyl 4-((5-(5-chloro-2-fluorophenyl)-2-isopropyl-6-oxo-1,6-dihydropyridin-3-yl)amino)-5-fluoro-1H-pyrrolo[2,3-b]pyridine-1-carboxylate (250 mg, 27.35%) as brown solid. LCMS: (M+1)⁺515.2

### Step-3: Synthesis 3-(5-chloro-2-fluorophenyl)-5-((5-fluoro-1H-pyrrolo[2,3-b]pyridin-4-yl)amino)-6-isopropylpyridin-2(1H)-one

tert-butyl 4-((5-(5-chloro-2-fluorophenyl)-2-isopropyl-6-oxo-1,6-dihydropyridin-3-yl)amino)-5-fluoro-1H-pyrrolo[2,3-b]pyridine-1-carboxylate (0.240 g, 0.466 mmol, 1.0 eq) was dissolved in 4.0 M-HCl in dioxane (3.0 mL) at rt. The resulting mixture was stirred at the rt for overnight. The progress of reaction was monitored by LCMS. The reaction mixture was neutralized with saturated solution of NaHCO₃ (100 mL), extracted with EtOAc (2×100 mL), the combined organic layers were washed with water (50.0 mL), with brine (50.0 mL), dried over Na₂SO₄ concentrated and purified by combi flash [silica gel 100-200 mesh; elution 0-5% MeOH in DCM] to afford the desired compound of 3-(5-chloro-2-fluorophenyl)-5-((5-fluoro-1H-pyrrolo[2,3-b]pyridin-4-yl)amino)-6-isopropylpyridin-2(1H)-one (60 mg, 31.25%) as brown solid. LCMS: (M+1)⁺415.3; ¹H NMR (400 MHz, DMSO-d₆): δ 11.86 (brs, 1H) 11.31 (brs, 1H) 7.92-7.99 (m, 2H) 7.53 (d, J=3.51 Hz, 1H) 7.48 (s, 2H) 7.36-7.45 (m, 1H) 7.22-7.29 (m, 1H) 7.11 (brs, 1H) 5.67 (brs, 1H) 3.25 (brs, 1H) 1.16 (d, J=7.02 Hz, 6H).

### Example-58: Synthesis of N-(4-((5-(3-chloro-4-fluorophenyl)-2-isopropyl-6-oxo-1,6-dihydropyridin-3-yl)amino)pyridin-2-yl)acetamide (Compound 1.58)

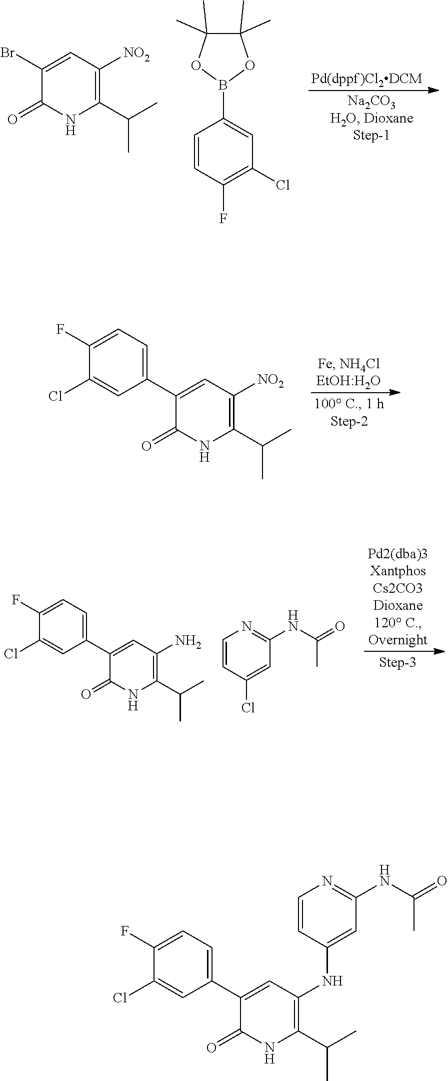

### Step-1: Synthesis 3-(3-chloro-4-fluorophenyl)-6-isopropyl-5-nitropyridin-2(1H)-one

To a stirred solution of 3-bromo-6-isopropyl-5-nitropyridin-2(1H)-one (0.500 g, 1.915 mmol, 1.0 eq), and 2-(3-chloro-4-fluorophenyl)-4,4,5,5-tetramethyl-1,3,2-dioxaborolane (0.540 g, 2.106 mmol, 1.1 eq) in dioxane (10.0 mL) was added a 2 M solution of sodium carbonate (0.406 g, 3.83 mmol, 2.0 eq) in water (1.9 mL) at rt. The resulting mixture was purged with nitrogen for 10 minute followed by addition of Pd(dppf)Cl₂.DCM (0.032 g, 0.033 mmol, 0.02 eq), again purged with nitrogen for 10 minute. The resulting mixture was heated at 120° C. for overnight. The progress of reaction was monitored by LCMS. The reaction mixture was diluted with EtOAc (20 mL), washed with water (20 mL), with brine (20 mL), dried over Na₂SO₄, concentrated and purified by combi flash chromatography [silica gel 100-200 mesh: elution 0-30% EtOAc in Hexane] to afford the desired compound 3-(3-chloro-4-fluorophenyl)-6-isopropyl-5-nitropyridin-2(1H)-one (0.4 g, 67.22%) as brown solid. LCMS: (M+1)⁺311.1

### Step-2: Synthesis of 5-amino-3-(3-chloro-4-fluorophenyl)-6-isopropylpyridin-2(1H)-one

To a stirred solution of 3-(3-chloro-4-fluorophenyl)-6-isopropyl-5-nitropyridin-2(1H)-one (0.400 g, 1.287 mmol, 1.0 eq) in EtOH (15.0 mL) and water (15.0 mL) was added Fe (0.575 g, 10.298 mmol, 8.0 eq) and NH₄Cl (0.688 g, 12.87 mmol, 10.0 eq) at rt. The resulting mixture was heated at 100° C. for 60 min. The progress of reaction was monitored by LCMS. The reaction mixture was filtered through celite the residue was washed with EtOH (50 mL) the filtrate was concentrated and the residue was dissolved in EtOAc (100 mL), washed with water (50 mL), dried over Na₂SO₄, and concentrated to afford the desired 5-amino-3-(3-chloro-4-fluorophenyl)-6-isopropylpyridin-2(1H)-one (0.370 g, 99.72%) as brown solid. LCMS: (M+1)⁺: 281.1

### Step-3: Synthesis of N-(4-((5-(3-chloro-4-fluorophenyl)-2-isopropyl-6-oxo-1,6-dihydropyridin-3-yl)amino)pyridin-2-yl)acetamide

To a stirred solution of compound 5-amino-3-(3-chloro-4-fluorophenyl)-6-isopropylpyridin-2(1H)-one (0.370 g, 1.318 mmol, 1.0 eq) and N-(4-chloropyridin-2-yl)acetamide (0.247 g, 1.449 mmol, 1.1 eq) in dioxane (10 mL) was added Cs₂CO₃ (1.30 g, 3.954 mmol, 3.0 eq) at rt. The resulting mixture was purged with nitrogen for 10 min followed by addition of Pd₂(dba)₃ (0.145 g, 0.158 mmol, 0.12 eq) and xantphos (0.115 g, 0.197 mmol, 0.15 eq), again purged with nitrogen for 10 min. The reaction mixture was heated at 120° C. for overnight. The progress of reaction was monitored by LCMS. The reaction mixture was diluted with EtOAc (100 mL), washed with water (50 mL), with brine (50 mL), dried over Na₂SO₄, concentrated and purified by combi flash chromatography [silica gel 100-200 mesh: elution 0-10% MeOH in DCM] to afford the desired compound N-(4-((5-(3-chloro-4-fluorophenyl)-2-isopropyl-6-oxo-1,6-dihydropyridin-3-yl)amino)pyridin-2-yl)acetamide (33 mg, 6.05%) as brown solid. LCMS: (M+1)⁺: 415.3;

¹H NMR (400 MHz, DMSO-d₆) δ ppm 11.79 (brs, 1H) 10.10 (brs, 1H) 8.05 (d, J=5.70 Hz, 2H) 7.73-7.86 (m, 2H) 7.57 (s, 1H) 7.39 (d, J=8.77 Hz, 2H) 6.24 (brs, 1H) 2.98-3.10 (m, 1H) 2.01 (s, 3H) 1.20 (d, J=6.58 Hz, 6H).

### Example-59: Synthesis of 5-((5-(5-chloro-2-fluorophenyl)-2-isopropyl-6-oxo-1,6-dihydropyridin-3-yl)amino)-1,8-naphthyridin-4(1H)-one (Compound 1.59)

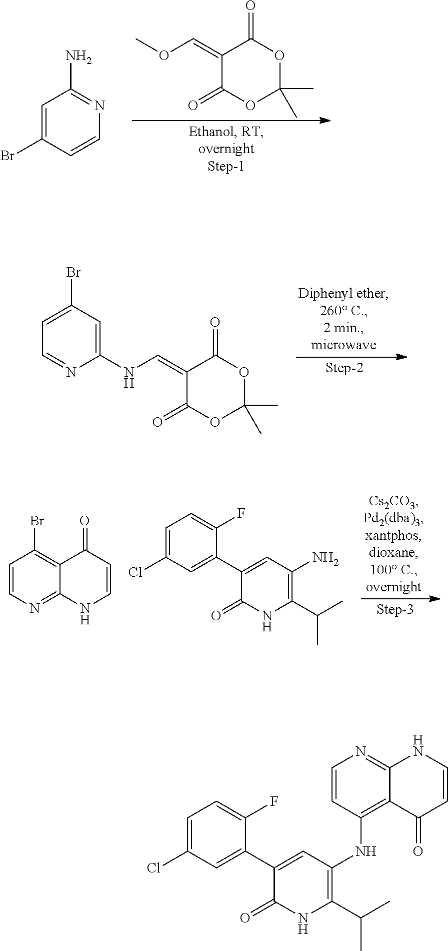

### Step-1: Synthesis of 5-(((4-bromopyridin-2-yl)amino)methylene)-2,2-dimethyl-1,3-dioxane-4,6-dione

To a solution of 4-bromopyridin-2-amine (200 mg, 1.16 mmol, 1 eq) in ethanol (5 mL), was added 5-(methoxymethylene)-2,2-dimethyl-1,3-dioxane-4,6-dione (216 mg, 1.16 mmol, 1 eq). The reaction mixture was then allowed to stir at rt for overnight. Progress of the reaction was monitored by TLC and LCMS. After completion of the reaction, solvent was removed completely under reduced pressure to obtain (300 mg, 79%) as an off white color solid compound. ¹H NMR (DMSO-d₆, 400 MHz): δ 11.37 (d, J=13.6 Hz, 1H), 9.20 (d, J=13.6 Hz, 1H), 8.33 (d, J=5.3 Hz, 1H), 8.04 (d, J=1.3 Hz, 1H), 7.55 (dd, J=5.3, 1.8 Hz, 1H), 1.68 ppm (s, 6H).

### Step-2: Synthesis of 5-bromo-1,8-naphthyridin-4(1H)-one

A solution of 5-(((4-bromopyridin-2-yl)amino)methylene)-2,2-dimethyl-1,3-dioxane-4,6-dione (250 mg, 0.76 mmol, 1 eq) in Diphenyl ether (2 mL) was allowed to stir at 260° C. for 2 min. in a microwave. Progress of the reaction was monitored by TLC and LCMS. After completion of the reaction, solid observed was filtered, washed with diethyl ether (20 mL) and dried under vacuum to obtain desired compound (200 mg, 32%) as an off white solid. LCMS: (M+1)⁺225.

### Step-3: Synthesis of 5-((5-(5-chloro-2-fluorophenyl)-2-isopropyl-6-oxo-1,6-dihydropyridin-3-yl)amino)-1,8-naphthyridin-4(1H)-one

To a solution of 5-amino-3-(5-chloro-2-fluorophenyl)-6-isopropylpyridin-2(1H)-one (150 mg, 0.53 mmol, 1 eq) in dioxane (5 mL), was added 5-bromo-1,8-naphthyridin-4(1H)-one (144 mg, 0.64 mmol, 1.2 eq) and cesium carbonate (346 mg, 1.06 mmol, 2 eq). The reaction mixture was degassed with nitrogen gas for 15 min., followed by the addition of Pd₂ (dba)₃ (24 mg, 0.026 mmol, 0.05 eq) and xantphos (31 mg, 0.053 mmol, 0.1 eq). The resultant reaction mixture was allowed to stir at 100° C. for overnight. Progress of the reaction was monitored by TLC and LCMS. After completion of the reaction, diluted with water (30 mL) and extracted with ethyl acetate (100 mL). Organic layer was washed with water (50 mL) and brine solution (50 mL). Organic layer was dried over anhydrous sodium sulphate and concentrated under reduced pressure to obtain crude, which was purified by reverse phase HPLC to obtain desired compound (20 mg, 9%) as an off white solid. LCMS: [M+1] ⁺425; ¹H NMR (DMSO-d₆,400 MHz): δ 11.94 (brs, 1H), 8.95 (brs, 1H), 8.77 (d, J=7.9 Hz, 1H), 7.99 (d, J=6.1 Hz, 1H), 7.64 (d, J=3.9 Hz, 1H), 7.54 (s, 1H), 7.36-7.47 (m, 1H), 7.30 (t, J=9.2 Hz, 1H), 6.93 (brs, 1H), 6.22 (brs, 1H), 5.90 (d, J=6.1 Hz, 1H), 3.01-3.17 (m, 1H), 1.06-1.30 (m, 6H).

### Example-60: Synthesis of 2-(4-((5-(5-chloro-2-fluorophenyl)-2-isopropyl-6-oxo-1,6-dihydropyridin-3-yl)amino)pyridin-2-yl)-2-methylpropanenitrile (Compound 1.60)

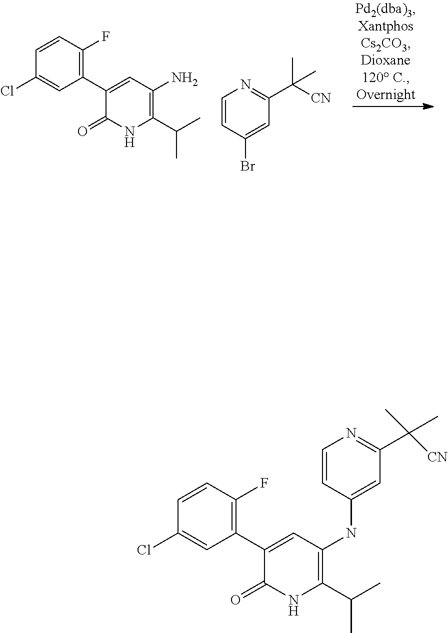

To a stirred solution of 5-amino-3-(5-chloro-2-fluorophenyl)-6-isopropylpyridin-2(1H)-one (0.20 g, 0.712 mmol, 1.0 eq) and 2-(4-bromopyridin-2-yl)-2-methylpropanenitrile (0.160 g, 0.712 mmol, 1.0 eq) in dioxane (10 mL) was added Cs₂CO₃ (0.696 g, 2.136 mmol, 3.0 eq) at rt. The resulting mixture was purged with nitrogen for 10 min followed by addition of Pd₂(dba)₃ (0.078 g, 0.085 mmol, 0.12 eq) and xantphos (0.062 g, 0.1025 mmol, 0.15 eq), again purged with nitrogen for 10 min. The reaction mixture was heated at 120° C. for overnight. The progress of reaction was monitored by LCMS. The reaction mixture was diluted with EtOAc (100 mL), washed with water (50 mL), with brine (50 mL), dried over Na₂SO₄, concentrated and purified by combi flash chromatography [silica gel 100-200 mesh: elution 0-10% MeOH in DCM] to afford the desired compound (0.06 g, 19.86%) as off white solid. LCMS: (M+1)⁺425.4; ¹H NMR (400 MHz, DMSO-d₆) δ 11.86 (brs, 1H) 8.29 (brs, 1H) 8.11 (d, J=5.70 Hz, 1H) 7.62 (d, J=6.14 Hz, 1H) 7.39-7.51 (m, 2H) 7.29 (t, J=9.21 Hz, 1H) 6.78 (brs, 1H) 6.43 (brs, 1H) 2.99-3.11 (m, 1H) 1.63 (s, 6H) 1.21 (d, J=7.02 Hz, 6H).

### Example-61: Synthesis of 4-((5-(5-chloro-2-fluorophenyl)-2-isopropyl-6-oxo-1,6-dihydropyridin-3-yl)amino)-1,3-dihydro-2H-pyrrolo[2,3-b]pyridin-2-one (Compound 1.61)

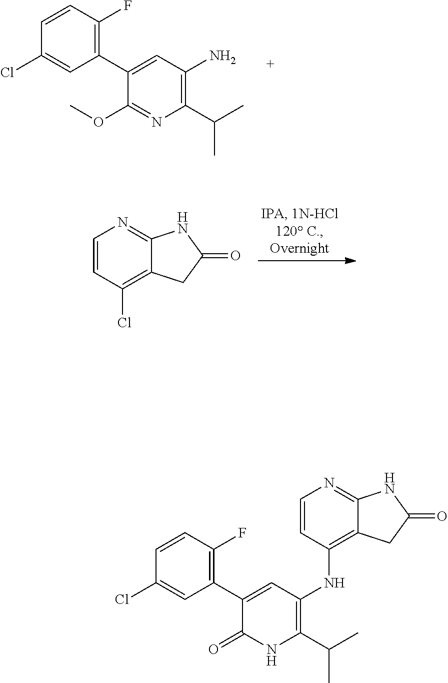 [SMILES: COc1nc(C(C)C)c(N)cc1-c1cc(Cl)ccc1F ; O=C1Cc2c(Cl)ccnc2N1 ; CC(C)c1[nH]c(=O)c(-c2cc(Cl)ccc2F)cc1Nc1ccnc2c1CC(=O)N2]

To a stirred solution of 5-(5-chloro-2-fluorophenyl)-2-isopropyl-6-methoxypyridin-3-amine (0.20 g, 0.678 mmol, 1.0 eq) and 4-chloro-1,3-dihydro-2H-pyrrolo[2,3-b]pyridin-2-one (0.115 g, 0.678 mmol, 1.0 eq) in isopropyl alcohol (2 mL) was added 1N—HCl (0.34 mL, 0.34 mmol, 0.5 eq) at rt. The reaction mixture was heated at 120° C. for overnight. The progress of reaction was monitored by LCMS. The reaction mixture was neutralized with saturated solution of NaHCO₃ (100 mL), extracted with EtOAc (2×50 mL). The combined organic layers were washed with brine (50 mL), dried over Na₂SO₄, concentrated and purified by combi flash chromatography [silica gel 100-200 mesh: elution 0-10% MeOH in DCM] to afford the desired compound (0.025 g, 8.92%) as off white solid. LCMS: (M+1)⁺413.3; ¹H NMR (400 MHz, DMSO-d₆): δ 11.84 (brs, 1H) 10.63 (s, 1H) 7.74 (brs, 1H) 7.68 (d, J=6.14 Hz, 1H) 7.63 (brs, 1H) 7.42 (s, 2H) 7.29 (t, J=9.43 Hz, 1H) 6.05 (d, J=5.70 Hz, 1H) 3.26 (brs, 2H) 3.15 (d, J=6.58 Hz, 1H) 1.20 (d, J=7.02 Hz, 6H).

### Example-62: Synthesis of 5-((5-(5-chloro-2-fluorophenyl)-2-isopropyl-6-oxo-1,6-dihydropyridin-3-yl)amino)-3,4-dihydro-1, 8-naphthyridin-2(1H)-one (Compound 1.62)

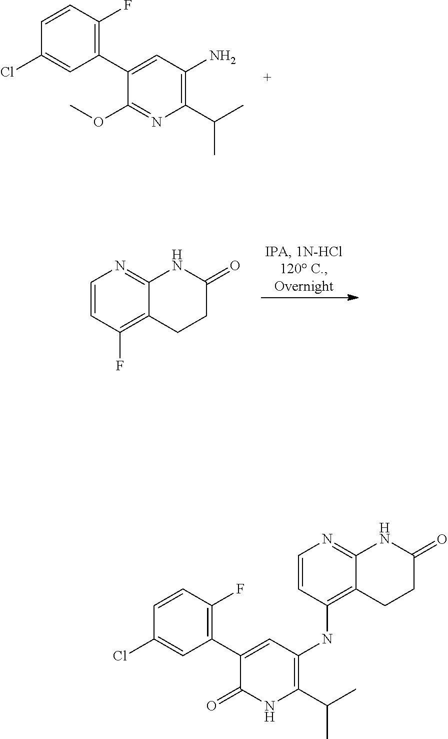 [SMILES: COc1nc(C(C)C)c(N)cc1-c1cc(Cl)ccc1F ; O=C1CCc2c(F)ccnc2N1 ; CC(C)c1[nH]c(=O)c(-c2cc(Cl)ccc2F)cc1Nc1ccnc2c1CCC(=O)N2]

To a stirred solution of 5-(5-chloro-2-fluorophenyl)-2-isopropyl-6-methoxypyridin-3-amine (0.20 g, 0.678 mmol, 1.0 eq) and 5-fluoro-3,4-dihydro-1,8-naphthyridin-2(1H)-one (0.115 g, 0.678 mmol, 1.0 eq) in isopropyl alcohol (2 mL) was added 1N—HCl (0.34 mL, 0.34 mmol, 0.5 eq) at rt. The reaction mixture was heated at 120° C. for overnight. The progress of reaction was monitored by LCMS. The reaction mixture was neutralized with saturated solution of NaHCO₃ (100 mL), extracted with EtOAc (2×50 mL). The combined organic layers were washed with brine (50 mL), dried over Na₂SO₄, concentrated and purified by combi flash chromatography [silica gel 100-200 mesh: elution 0-10% MeOH in DCM] to afford the desired compound (0.015 g, 5.17%) as brown solid. LCMS: (M+1)⁺427.3; ¹H NMR (400 MHz, DMSO-d₆): δ 11.85 (brs, 1H) 10.03 (s, 1H) 7.69 (d, J=5.70 Hz, 1H) 7.62 (dd, J=6.36, 2.41 Hz, 1H) 7.38-7.46 (m, 2H) 7.26-7.36 (m, 1H) 5.94 (d, J=5.70 Hz, 1H) 3.02-3.13 (m, 1H) 2.73-2.86 (m, 2H), 2.48-2.50 (m, 2H) 1.20 (d, J=7.02 Hz, 6H).

### Example-63: Synthesis of N-(4-((5-(5-chloro-2-fluorophenyl)-2-isopropyl-6-oxo-1,6-dihydropyridin-3-yl)amino)pyridin-2-yl)methanesulfonamide (Compound 1.63)

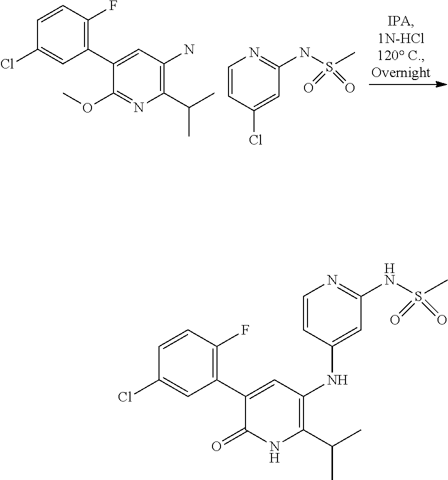

To a stirred solution of 5-(5-chloro-2-fluorophenyl)-2-isopropyl-6-methoxypyridin-3-amine (0.20 g, 0.678 mmol, 1.0 eq) and N-(4-chloropyridin-2-yl)methanesulfonamide (0.140 g, 0.678 mmol, 1.0 eq) in isopropyl alcohol (2 mL) was added 1N—HCl (0.34 mL, 0.34 mmol, 0.5 eq) at rt. The reaction mixture was heated at 120° C. for overnight. The progress of reaction was monitored by LCMS. The reaction mixture was neutralized with saturated solution of NaHCO₃(50 mL), extracted with EtOAc (2×50 mL). The combined organic layers were washed with brine (50 mL), dried over Na₂SO₄, concentrated and purified by reverse phase purification to afford the desired compound (0.005 g, 1.73%) as off white solid. LCMS: (M+1)⁺451.3; ¹H NMR (400 MHz, DMSO-d₆): δ8.71 (brs, 1H) 8.38 (s, 1H) 7.50-7.61 (m, 1H) 7.36-7.49 (m, 3H) 7.27 (t, J=9.21 Hz, 1H) 6.10 (s, 2H) 2.99-3.04 (m, 1H) 2.72 (brs, 3H) 1.19 (d, J=7.02 Hz, 6H).

### Example-64: Synthesis of N-(4-((5-(5-chloro-2-fluoro-4-methylphenyl)-2-isopropyl-6-oxo-1,6-dihydropyridin-3-yl)amino)pyridin-2-yl)acetamide (Compound 1.64)

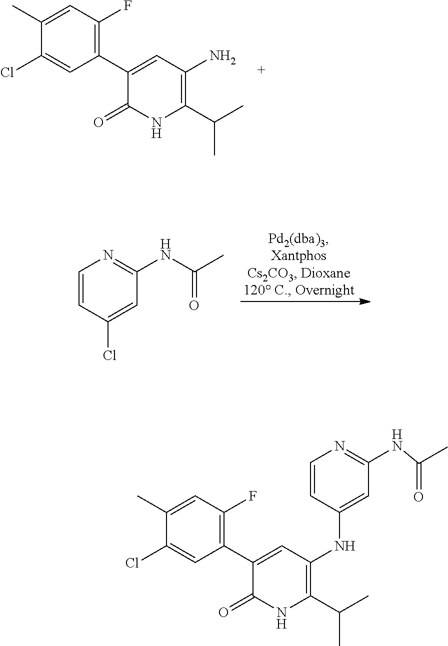 [SMILES: Cc1cc(F)c(-c2cc(N)c(C(C)C)[nH]c2=O)cc1Cl ; CC(=O)Nc1cc(Cl)ccn1 ; CC(=O)Nc1cc(Nc2cc(-c3cc(Cl)c(C)cc3F)c(=O)[nH]c2C(C)C)ccn1]

To a stirred solution of 5-amino-3-(5-chloro-2-fluorophenyl)-6-isopropylpyridin-2(1H)-on 5-amino-3-(5-chloro-2-fluoro-4-methylphenyl)-6-isopropylpyridin-2(1H)-one (0.415 g, 1.407 mmol, 1.0 eq) and N-(4-chloropyridin-2-yl)acetamide (0.264 g, 1.548 mmol, 1.0 eq) in dioxane (20 mL) was added Cs₂CO₃ (1.37 g, 4.221 mmol, 3.0 eq) at rt. The resulting mixture was purged with nitrogen for 10 min followed by addition of Pd₂(dba)₃ (0.154 g, 0.168 mmol, 0.12 eq) and xantphos (0.122 g, 0.219 mmol, 0.15 eq), again purged with nitrogen for 10 min. The reaction mixture was heated at 120° C. for overnight. The progress of reaction was monitored by LCMS. The reaction mixture was diluted with EtOAc (100 mL), washed with water (50 mL), with brine (50 mL), dried over Na₂SO₄, concentrated and purified by combi flash chromatography [silica gel 100-200 mesh: elution 0-5% MeOH in DCM] to afford the desired compound (0.015 g, 2.48%) as off white solid. LCMS: (M+1)⁺: 429.4; ¹H NMR (400 MHz, DMSO-d₆): δ11.75 (brs, 1H) 10.08 (s, 1H) 8.24 (brs, 1H) 8.04 (s, 1H) 7.82 (d, J=5.70 Hz, 1H) 7.61 (d, J=7.02 Hz, 1H) 7.33-7.42 (m, 1H) 7.27 (s, 1H) 6.21 (brs, 1H) 3.03-3.11 (m, 1H) 2.34 (s, 3H) 2.01 (s, 3H) 1.20 (d, J=6.58 Hz, 6H).

### Example-65: Synthesis of N-(4-((5-(5-chloro-2-fluorophenyl)-2-isopropyl-6-oxo-1,6-dihydropyridin-3-yl)amino)pyridin-2-yl)-2-morpholinoacetamide (Compound 1.65)

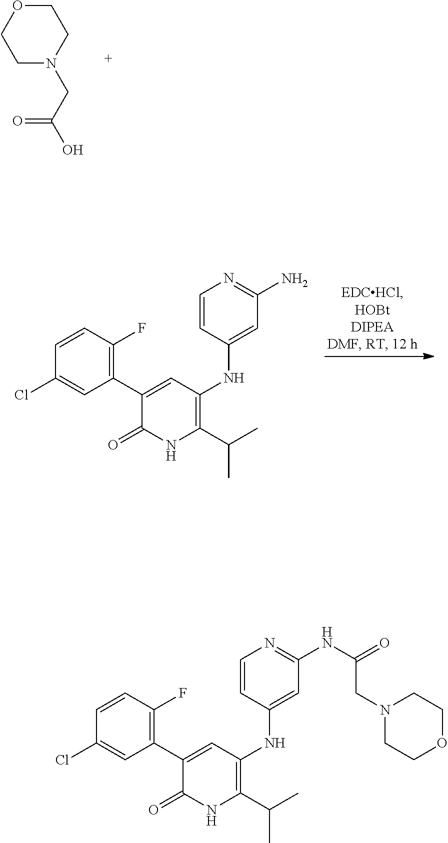 [SMILES: O=C(O)CN1CCOCC1 ; CC(C)c1[nH]c(=O)c(-c2cc(Cl)ccc2F)cc1Nc1ccnc(N)c1 ; CC(C)c1[nH]c(=O)c(-c2cc(Cl)ccc2F)cc1Nc1ccnc(NC(=O)CN2CCOCC2)c1]

To a stirred solution of 2-morpholinoacetic acid (0.035 g, 0.241 mmol, 1.0 eq) and 5-((2-aminopyridin-4-yl)amino)-3-(5-chloro-2-fluorophenyl)-6-isopropylpyridin-2(1H)-one (0.090 g, 0.241 mmol, 1.0 eq) in DMF (2 mL) was added HOBt (0.048 g, 0.361 mmol, 1.5 eq) and EDC.HCl (0.070 g, 0.361 mmol, 1.5 eq) followed by addition of and DIPEA (0.12 mL, 0.723 mmol, 3.0 eq) and stirred at rt for overnight. Reaction mixture was diluted with water (50 mL) and extracted with ethyl acetate (2×50 mL). The combined organic were washed with water (50 mL), with brine (50 mL) dried over Na₂SO₄ and concentrated under reduced and purified prep TLC to afford the desired compound (0.006 g, %) as brown solid. LCMS: (M+1)⁺500.4; ¹H NMR (400 MHz, DMSO-d₆): δ 13.54 (s, 1H) 12.31 (brs, 1H) 10.50 (d, J=6.14 Hz, 1H) 7.96 (d, J=7.02 Hz, 2H) 7.82 (brs, 1H) 7.68 (dd, J=6.36, 2.41 Hz, 1H) 7.51 (dd, J=8.33, 3.51 Hz, 1H) 7.35 (t, J=9.43 Hz, 1H) 7.05 (d, J=6.14 Hz, 1H) 6.88 (brs, 1H) 6.55 (s, 1H) 4.04 (brs, 2H) 3.82 (brs, 4H) 3.50 (brs, 2H) 2.71-2.89 (m, 2H) 1.16-1.34 (m, 6H).

### Example-66: Synthesis of N-(4-((5-(5-chloro-2-fluorophenyl)-2-isopropyl-6-oxo-1,6-dihydropyridin-3-yl)(methyl)amino)pyridin-2-yl)acetamide (Compound 1.66)

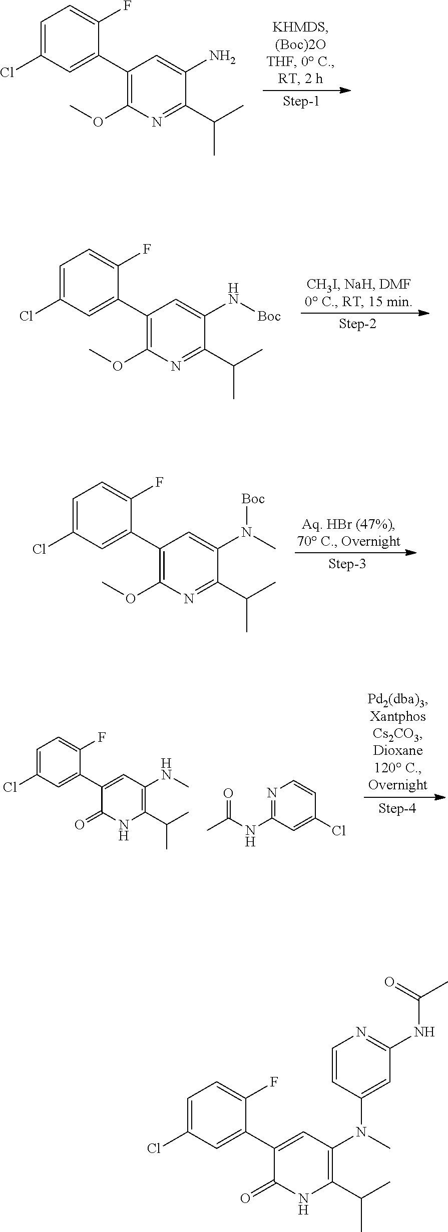

### Step-1: Synthesis of tert-butyl (5-(5-chloro-2-fluorophenyl)-2-isopropyl-6-methoxypyridin-3-yl)carbamate

To stirred solution of 5-(5-chloro-2-fluorophenyl)-2-isopropyl-6-methoxypyridin-3-amine (1.0 g, 3.352 mmol, 1.0 eq) in THF (20.0 mL) was added KHMDS (7.462 mL, 7.462 mmol, 2.2 eq) at 0° C. The resulting mixture was stirred at the same temperature for 5 min. followed by addition of (Boc)₂0 (0.86 mL, 3.731 mmol, 1.1 eq). The resulting mixture was stirred at rt for 2 h. The progress of reaction was monitored by LCMS. The reaction mixture was quenched with saturated solution of NH₄Cl solution (50.0 mL), extracted with EtOAc (2×100 mL), the combined organic layers were washed with water (50.0 mL), with brine (50.0 mL), dried over Na₂SO₄, concentrated and purified by combiflash [silica gel 100-200 mesh elution 0-50% EtOAc in hexane] to afford the desired (0.50 g, 37.59%) as brown solid. LCMS: (M+1)⁺395.1

### Step-2: Synthesis of tert-butyl (5-(5-chloro-2-fluorophenyl)-2-isopropyl-6-methoxypyridin-3-yl)(methyl)carbamate

To stirred solution of tert-butyl (5-(5-chloro-2-fluorophenyl)-2-isopropyl-6-methoxypyridin-3-yl)carbamate (0.50 g, 1.266 mmol, 1.0 eq) in DMF (2.0 mL) was added NaH (0.10 g, 2.532 mmol, 2.0 eq) at 0° C. The resulting mixture was stirred at the same temperature for 5 min. followed by addition of CH₃I (0.12 mL, 1.899 mmol, 1.1 eq). The resulting mixture was stirred at rt for 15 min. The progress of reaction was monitored by LCMS. The reaction mixture was diluted with water (50.0 mL), extracted with EtOAc (2×50 mL), the combined organic layers were washed with water (50.0 mL), with brine (50.0 mL), dried over Na₂SO₄, concentrated to afford the desired (0.45 g, 87.04%) as brown solid. LCMS: (M+1)⁺409.1

### Step-3: Synthesis of 3-(5-chloro-2-fluorophenyl)-6-isopropyl-5-(methylamino)pyridin-2(1H)-one

tert-butyl (5-(5-chloro-2-fluorophenyl)-2-isopropyl-6-methoxypyridin-3-yl)(methyl)carbamate (0.45 g, 1.100 mmol, 1.0 eq) and aq. HBr (47%) (2.0 mL) were stirred at 70° C. for overnight. The progress of reaction was monitored by LCMS. The reaction mixture was neutralized with with saturated solution of NaHCO₃ (100.0 mL), extracted with EtOAc (2×50 mL), the combined organic layers were washed with water (50.0 mL), with brine (50.0 mL), dried over Na₂SO₄, concentrated to afford the desired compound (0.250 g, 76.92%) as brown solid. LCMS: (M+1)⁺: 295.1

### Step-4: Synthesis of N-(4-((5-(5-chloro-2-fluorophenyl)-2-isopropyl-6-oxo-1,6-dihydropyridin-3-yl)(methyl)amino)pyridin-2-yl)acetamide

To a stirred solution of 3-(5-chloro-2-fluorophenyl)-6-isopropyl-5-(methylamino)pyridin-2(1H)-one (0.230 g, 0.780 mmol, 1.0 eq) and N-(4-chloropyridin-2-yl)acetamide (0.145 g, 0.858 mmol, 1.1 eq) in dioxane (10.0 mL) was added Cs₂CO₃ (0.763 g, 2.34 mmol, 3.0 eq) at rt. The resulting mixture was purged with nitrogen for 10 min followed by addition of Pd₂(dba)₃ (0.085 g, 0.094 mmol, 0.12 eq) and xantphos (0.068 g, 0.117 mmol, 0.15 eq), again purged with nitrogen for 10 min. The reaction mixture was heated at 120° C. for overnight. The progress of reaction was monitored by LCMS. The reaction mixture was diluted with water (50.0 mL), extracted with EtOAc (2×50 mL), the combined organic layers were washed with water (50.0 mL), with brine (50.0 mL), dried over Na₂SO₄, concentrated and purified by combiflash [silica gel 100-200 mesh elution 0-10% MeOH in DCM] to afford the desired compound (0.01 g, 2.99%) as off white solid. LCMS: (M+1)⁺429.4; ¹H NMR (400 MHz, DMSO-d₆) δ 13.36 (brs, 1H) 12.14 (brs, 1H) 11.37 (brs, 1H) 7.96 (brs, 1H) 7.52-7.63 (m, 2H) 7.45-7.52 (m, 1H) 7.32 (t, J=9.21 Hz, 1H) 2.71-2.83 (m, 1H) 2.15 (brs, 3H) 1.33 (brs, 1H) 1.12-1.30 (m, 6H).

### Example-67: Synthesis of 5-((1,6-naphthyridin-4-yl)amino)-3-(5-chloro-2-fluorophenyl)-6-cyclopropylpyridin-2(1H)-one (Compound 1.67)

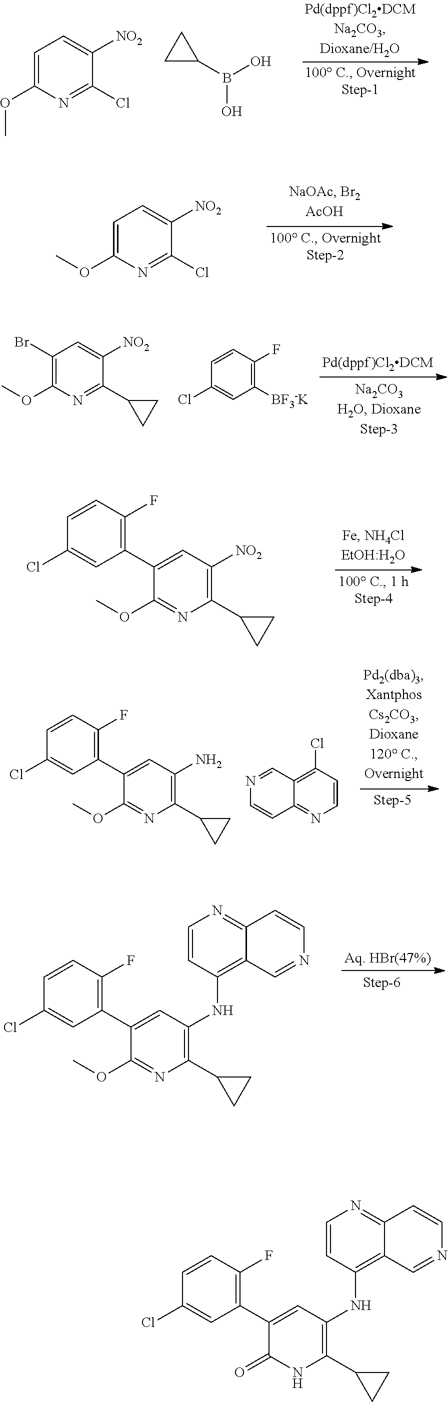

### Step-1: Synthesis 2-cyclopropyl-6-methoxy-3-nitropyridine

To a stirred solution of 2-chloro-6-methoxy-3-nitropyridine (10.0 g, 53.03 mmol, 1.0 eq), and cyclopropylboronic acid (5.50 g, 63.636 mmol, 1.2 eq) in dioxane (50.0 mL) was added a 2 M solution of sodium carbonate (11.25 g, 106.06 mmol, 2.0 eq) in water (53.0 mL) at rt. The resulting mixture was purged with nitrogen for 10 minute followed by addition of Pd(dppf)Cl₂.DCM (0.87 g, 1.06 mmol, 0.02 eq), again purged with nitrogen for 10 minute. The resulting mixture was heated at 120° C. for overnight. The progress of reaction was monitored by LCMS. The reaction mixture was diluted with EtOAc (500 mL), washed with water (100 mL), with brine (100 mL), dried over Na₂SO₄, concentrated and purified by combi flash chromatography [silica gel 100-200 mesh: elution 100% Hexane] to afford the desired compound (7.80 g, 75.72%) as yellow solid. LCMS: (M+1)⁺194.9

### Step-2: Synthesis of 3-bromo-6-cyclopropyl-2-methoxy-5-nitropyridine

To a stirred solution of 2-cyclopropyl-6-methoxy-3-nitropyridine (0.750 g, 3.861 mmol, 1.0 eq) in AcOH (10 mL) was added NaOAc (1.27 g, 15.444 mmol, 4.0 eq) at rt, followed by dropwise addition of bromine (0.4 mL, 7.722 mmol, 2.0 eq). The resulting mixture was heated at 100° C. for overnight. The progress of reaction was monitored by ¹HNMR. The reaction mixture was neutralized with saturated solution of NaHCO₃ (200.0 mL) extracted with EtOAc (2×100 mL). The combined organic layers were washed with water (50 mL), with sodiumthiosulphate solution (200 mL), dried over Na₂SO₄, concentrated and purified by combi flash chromatography [silica gel 100-200 mesh: elution 100% Hexane] to afford the desired compound (0.45 g, 42.65%) as yellow solid. ¹H NMR (400 MHz, CDCl₃): δ 8.43 (s, 1H) 4.01 (s, 3H) 2.91-3.02 (m, 1H) 1.22-1.27 (m, 2H) 1.13-1.17 (m, 2H).

### Step-3: Synthesis 3-(5-chloro-2-fluorophenyl)-6-cyclopropyl-2-methoxy-5-nitropyridine

To a stirred solution of 3-bromo-6-cyclopropyl-2-methoxy-5-nitropyridine (0.45 g, 1.647 mmol, 1.0 eq), and potassium, (5-chloro-2-fluorophenyl)trifluoroborate (0.506 g, 2.142 mmol, 1.1 eq) in dioxane (20.0 mL) was added a 2 M solution of sodium carbonate (0.350 g, 3.293 mmol, 2.0 eq) in water (2.0 mL) at rt. The resulting mixture was purged with nitrogen for 10 minute followed by addition of Pd(dppf)Cl₂.DCM (0.027 g, 0.033 mmol, 0.02 eq), again purged with nitrogen for 10 minute. The resulting mixture was heated at 120° C. for overnight. The progress of reaction was monitored by LCMS. The reaction mixture was diluted with EtOAc (20 mL), washed with water (20 mL), with brine (20 mL), dried over Na₂SO₄, concentrated and purified by combi flash chromatography [silica gel 100-200 mesh: elution 100% Hexane] to afford the desired compound (0.440 g, 82.70%) as yellow solid. LCMS: (M+1)⁺322.9

### Step-4: Synthesis of 5-(5-chloro-2-fluorophenyl)-2-cyclopropyl-6-methoxypyridin-3-amine

To a stirred solution of 3-(5-chloro-2-fluorophenyl)-6-cyclopropyl-2-methoxy-5-nitropyridine (0.440 g, 1.363 mmol, 1.0 eq) in EtOH (15.0 mL) and water (15.0 mL) was added Fe (0.610 g, 10.907 mmol, 8.0 eq) and a solution of NH₄Cl (0.73 g, 13.63 mmol, 10.0 eq) at rt. The resulting mixture was heated at 100° C. for 60 min. The progress of reaction was monitored by LCMS. The reaction mixture was filtered through celite the residue was washed with EtOH (50 mL) the filtrate was concentrated and the residue was dissolved in EtOAc (50 mL), washed with water (2×50 mL), dried over Na₂SO₄, and concentrated to afford the desired compound (0.350 g, 87.72%) as brown brown. LCMS: (M+1)⁺293.1

### Step-5: Synthesis of N-(5-(5-chloro-2-fluorophenyl)-2-cyclopropyl-6-methoxypyridin-3-yl)-1,6-naphthyridin-4-amine

To a stirred solution of compound 5-(5-chloro-2-fluorophenyl)-2-cyclopropyl-6-methoxypyridin-3-amine (0.150 g, 0.512 mmol, 1.0 eq) and 4-chloro-1,6-naphthyridine (0.093 g, 0.563 mmol, 1.1 eq) in dioxane (10 mL) was added Cs₂CO₃ (0.508 g, 1.536 mmol, 3.0 eq) at rt. The resulting mixture was purged with nitrogen for 10 min followed by addition of Pd₂(dba)₃ (0.056 g, 0.062 mmol, 0.12 eq) and xantphos (0.041 g, 0.0716 mmol, 0.15 eq), again purged with nitrogen for 10 min. The reaction mixture was heated at 120° C. for overnight. The progress of reaction was monitored by LCMS. The reaction mixture was diluted with EtOAc (50 mL), washed with water (50 mL), with brine (50 mL), dried over Na₂SO₄, concentrated and purified by combi flash chromatography [silica gel 100-200 mesh: elution 0-10% MeOH in DCM] to afford the desired compound (135 mg, 62.79%) as brown solid. LCMS: (M+1)⁺421.1.

### Step-6: Synthesis of 5-((1,6-naphthyridin-4-yl)amino)-3-(5-chloro-2-fluorophenyl)-6-cyclopropylpyridin-2(1H)-one

N-(5-(5-chloro-2-fluorophenyl)-2-cyclopropyl-6-methoxypyridin-3-yl)-1,6-naphthyridin-4-amine (0.13 g, 0.308 mmol, 1.0 eq) was dissolved in aq. HBr (47%) (2.0 mL) at rt. The reaction mixture was stirred at 70° C. for 4 h. The progress of reaction was monitored by LCMS. The reaction mixture was basified with saturated aqueous solution of NaHCO₃(100 mL), extracted with EtOAc (2×100 mL). The combined organic layers were washed with water (50 mL), with brine (50 mL), dried over Na₂SO₄, and concentrated and purified by combi flash chromatography [silica gel 100-200 mesh: elution 0-10% MeOH in DCM] to afford the desired compound (0.038 g, 30.15%) as yellow solid. LCMS: (M+1)⁺: 407.3; H NMR (400 MHz, DMSO-d₆): δ11.36 (brs, 1H) 9.78 (s, 1H) 9.25 (brs, 1H) 8.62 (d, J=6.14 Hz, 1H) 8.53 (d, J=5.26 Hz, 1H) 7.67 (d, J=5.70 Hz, 1H) 7.51-7.64 (m, 2H) 7.44 (ddd, J=8.66, 4.28, 2.85 Hz, 1H) 7.30 (t, J=9.21 Hz, 1H) 6.37 (d, J=5.26 Hz, 1H) 1.96-2.08 (m, 1H) 1.03-1.13 (m, 2H) 0.85-0.94 (m, 2H).

### Example-68: Synthesis of 4-((5-(5-chloro-2-fluorophenyl)-2-cyclopropyl-6-oxo-1,6-dihydropyridin-3-yl)amino)-N-methylnicotinamide (Compound 1.68)

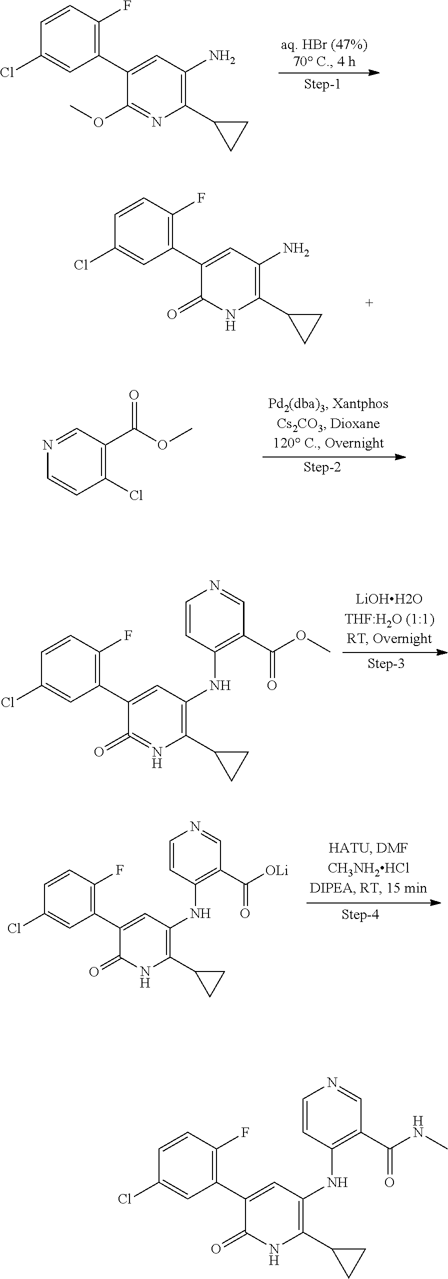

### Step-1: Synthesis of 5-amino-3-(5-chloro-2-fluorophenyl)-6-cyclopropylpyridin-2(1H)-one

5-(5-chloro-2-fluorophenyl)-2-cyclopropyl-6-methoxypyridin-3-amine (2.5 g, 8.54 mmol, 1.0 eq) was dissolved in aq. HBr (47%) (5.0 mL) at rt. The reaction mixture was stirred at 70° C. for 4 h. The progress of reaction was monitored by LCMS. The reaction mixture was basified with saturated aqueous solution of NaHCO₃(500 mL), extracted with EtOAc (2×100 mL). The combined organic layers were washed with water (50 mL), with brine (50 mL), dried over Na₂SO₄, and concentrated to afford the desired compound (2.10 g, 88.60%) as brown solid. LCMS: (M+1)⁺279.1

### Step-2: Synthesis of methyl 4-((5-(5-chloro-2-fluorophenyl)-2-cyclopropyl-6-oxo-1,6-dihydropyridin-3-yl)amino)nicotinate

To a stirred solution of compound 5-amino-3-(5-chloro-2-fluorophenyl)-6-cyclopropylpyridin-2(1H)-one (0.50 g, 1.783 mmol, 1.0 eq) and methyl 4-chloronicotinate (0.340 g, 1.973 mmol, 1.1 eq) in dioxane (20 mL) was added Cs₂CO₃ (1.75 g, 5.35 mmol, 3.0 eq) at rt. The resulting mixture was purged with nitrogen for 10 min followed by addition of Pd₂(dba)₃ (0.195 g, 0.213 mmol, 0.12 eq) and xantphos (0.155 g, 0.267 mmol, 0.15 eq), again purged with nitrogen for 10 min. The reaction mixture was heated at 120° C. for overnight. The progress of reaction was monitored by LCMS. The reaction mixture was diluted with EtOAc (50 mL), washed with water (50 mL), with brine (50 mL), dried over Na₂SO₄, concentrated and purified by combi flash chromatography [silica gel 100-200 mesh: elution 0-10% MeOH in DCM] to afford the desired compound (350 mg, 47.23%) as brown solid. LCMS: (M+1)⁺414.2

### Step-3: Synthesis of lithium 4-((5-(5-chloro-2-fluorophenyl)-2-cyclopropyl-6-oxo-1,6-dihydropyridin-3-yl)amino)nicotinate

To a stirred solution of methyl 4-((5-(5-chloro-2-fluorophenyl)-2-cyclopropyl-6-oxo-1,6-dihydropyridin-3-yl)amino)nicotinate (0.350 g, 0.845 mmol, 1.0 eq) in THF (16 mL) and H2O (4.0 mL) was added LiOH.H₂O (0.071 g, 1.691 mmom, 2.0 eq) at rt. The reaction mixture was stirred at rt for overnight. The progress of reaction was monitored by LCMS. The reaction mixture was concentrated to afford the desired compound (0.33 g, 96.20%) as brown solid. LCMS: (M+1)⁺400.1

### Step-4: Synthesis of 4-((5-(5-chloro-2-fluorophenyl)-2-cyclopropyl-6-oxo-1,6-dihydropyridin-3-yl)amino)-N-methylnicotinamide

To a stirred solution of lithium 4-((5-(5-chloro-2-fluorophenyl)-2-cyclopropyl-6-oxo-1, 6-dihydropyridin-3-yl)amino)nicotinate (0.20 g, 0.492 mmol, 1.0 eq) in DMF (2 mL) was added HATU (0.280 g, 0.739 mmol, 1.5 eq) at rt. The resulting mixture was stirred at rt for 5 minutes, followed by addition of CH₃NH₂.HCl (0.050 g, 0.739 mmol, 1.5 eq) and DIPEA (0.26 mL, 1.476 mmol, 3.0 eq) and stirred at rt for 15 min. Reaction mixture was diluted with water (20 mL) and extracted with ethyl acetate (2×20 mL). The combined organic extracts were washed with water (20 mL), with brine (20 mL) dried over Na₂SO₄ and concentrated under reduced and purified by combi flash chromatography [silic gel 100-200 mesh; elution 0-10% MeOH in DCM] to afford the desired compound (0.027 g, 13.70%) as light yellow solid. LCMS: (M+1)⁺413.3; ¹H NMR (400 MHz, DMSO-d₆): δ 11.42 (brs, 1H) 10.19 (brs, 1H) 8.81 (brs, 1H) 8.66 (s, 1H) 8.20 (d, J=7.02 Hz, 1H) 7.59 (brs, 1H) 7.49 (brs, 1H) 7.43 (brs, 1H) 7.30 (t, J=9.21 Hz, 1H) 6.67 (d, J=6.58 Hz, 1H) 2.81 (d, J=4.39 Hz, 3H) 1.89 (brs, 1H) 1.06 (brs, 2H) 0.91 (d, J=5.70 Hz, 2H).

### Example-69: Synthesis of N-(4-((5-(5-chloro-2-fluorophenyl)-2-cyclopropyl-6-oxo-1,6-dihydropyridin-3-yl)amino)pyridin-2-yl)acetamide (Compound 1.69)

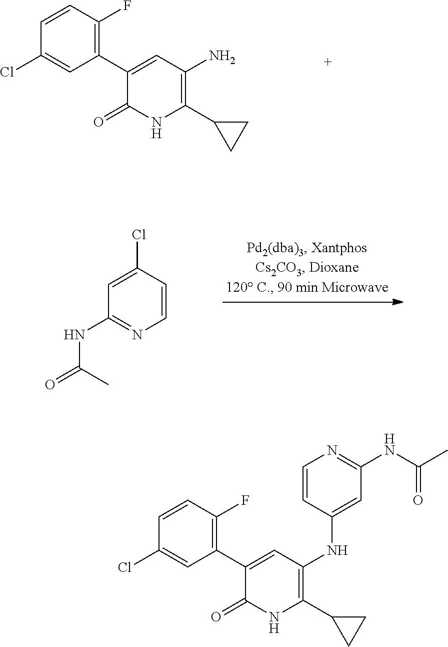 [SMILES: Nc1cc(-c2cc(Cl)ccc2F)c(=O)[nH]c1C1CC1 ; CC(=O)Nc1cc(Cl)ccn1 ; CC(=O)Nc1cc(Nc2cc(-c3cc(Cl)ccc3F)c(=O)[nH]c2C2CC2)ccn1]

To a stirred solution of compound 5-amino-3-(5-chloro-2-fluorophenyl)-6-cyclopropylpyridin-2(1H)-one (0.20 g, 0.717 mmol, 1.0 eq) and N-(4-chloropyridin-2-yl)acetamide (0.135 g, 789 mmol, 1.1 eq) in dioxane (2.0 mL) was added Cs₂CO₃ (0.70 g, 2.151 mmol, 3.0 eq) at rt. The resulting mixture was purged with nitrogen for 10 min followed by addition of Pd₂(dba)₃ (0.78 g, 0.086 mmol, 0.12 eq) and xantphos (0.062 g, 0.107 mmol, 0.15 eq), again purged with nitrogen for 10 min. The reaction mixture was heated at 120° C. for 90 min in microwave. The progress of reaction was monitored by LCMS. The reaction mixture was diluted with EtOAc (20 mL), washed with water (50 mL), with brine (50 mL), dried over Na₂SO₄, concentrated and purified by combi flash chromatography [silica gel 100-200 mesh: elution 0-5% MeOH in DCM] to afford the desired compound (0.019 g, 6.41%) as off white solid. LCMS: (M+1)⁺413.3; ¹H NMR (400 MHz, DMSO-d₆): δ 11.20 (brs, 1H) 10.12 (brs, 1H) 8.21 (brs, 1H) 7.83 (d, J=5.70 Hz, 1H) 7.59 (brs, 1H) 7.36-7.46 (m, 3H) 7.26-7.32 (m, 1H) 6.25 (brs, 1H) 2.02 (s, 4H) 1.06 (brs, 2H) 0.87-0.97 (m, 2H).

### Example-70: Synthesis of N-(4-((5-(5-chloro-2-fluorophenyl)-2-ethyl-6-oxo-1,6-dihydropyridin-3-yl)amino)pyridin-2-yl)acetamide (Compound 1.70)

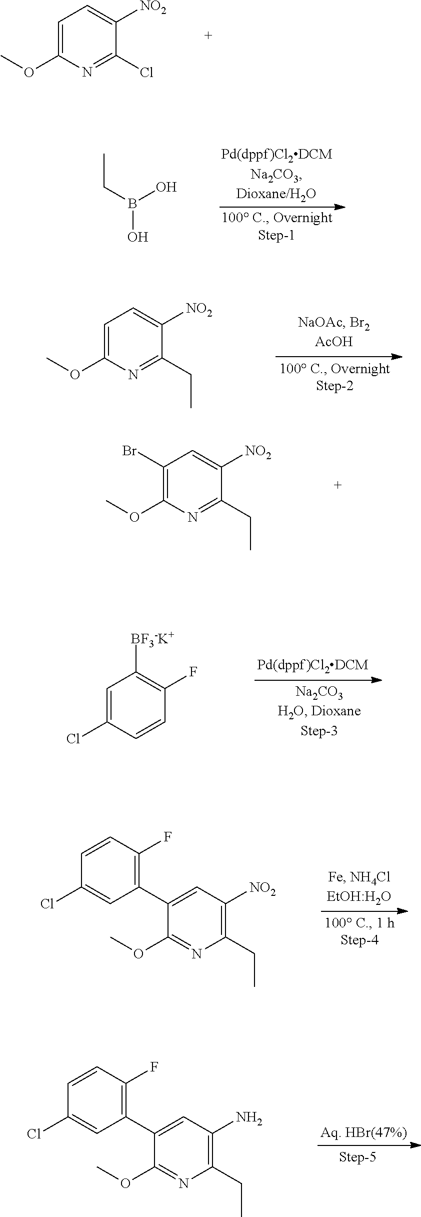

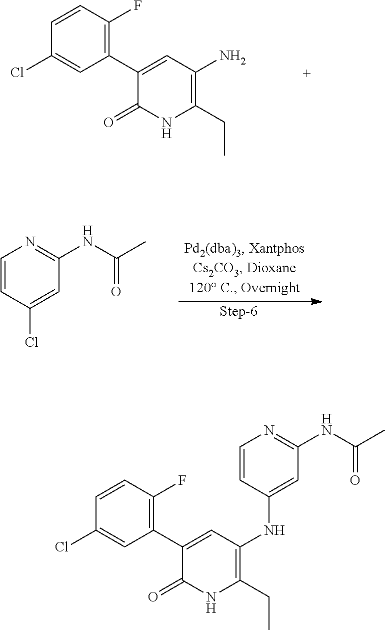 [SMILES: CCc1[nH]c(=O)c(-c2cc(Cl)ccc2F)cc1N ; CC(=O)Nc1cc(Cl)ccn1 ; CCc1[nH]c(=O)c(-c2cc(Cl)ccc2F)cc1Nc1ccnc(NC(C)=O)c1]

### Step-1: Synthesis 2-ethyl-6-methoxy-3-nitropyridine

To a stirred solution of 2-chloro-6-methoxy-3-nitropyridine (10.0 g, 53.03 mmol, 1.0 eq), and ethylboronic acid (4.70 g, 63.636 mmol, 1.2 eq) in dioxane (100.0 mL) was added a 2 M solution of sodium 5 carbonate (11.24 g, 106.06 mmol, 2.0 eq) in water (53.0 mL) at rt. The resulting mixture was purged with nitrogen for 10 minute followed by addition of Pd(dppf)Cl₂.DCM (0.87 g, 1.06 mmol, 0.02 eq), again purged with nitrogen for 10 minute. The resulting mixture was heated at 120° C. for overnight. The progress of reaction was monitored by LCMS. The reaction mixture was filtered through celite the residue was washed with EtOAc (100 mL), the filtrate was concentrated and purified by combi flash chromatography [silica gel 100-200 mesh: elution 100% Hexane] to afford the desired compound 2-ethyl-6-methoxy-3-nitropyridine (5.0 g, 51.75%) as light yellow solid. LCMS: (M+1)⁺182.9

### Step-2: Synthesis of 3-bromo-6-ethyl-2-methoxy-5-nitropyridine

To a stirred solution of 2-cyclopropyl-6-methoxy-3-nitropyridin2-ethyl-6-methoxy-3-nitropyridine (5.0 g, 27.445 mmol, 1.0 eq) in AcOH (50 mL) was added NaOAc (9.0 g, 109.78 mmol, 4.0 eq) at rt, followed by dropwise addition of bromine (2.83 mL, 54.89 mmol, 2.0 eq). The resulting mixture was heated at 100° C. for overnight. The progress of reaction was monitored by LCMS. The reaction mixture was neutralized with saturated solution of NaHCO₃(1.0 L) extracted with EtOAc (2×500 mL). The combined organic layers were washed with water (500 mL), with sodiumthiosulphate solution (500 mL), dried over Na₂SO₄, concentrated and purified by combi flash chromatography [silica gel 100-200 mesh: elution 100% Hexane] to afford the desired compound (1.75 g, 24.44%) as yellow solid. LCMS: (M+1)⁺261.1

### Step-3: Synthesis 3-(5-chloro-2-fluorophenyl)-6-ethyl-2-methoxy-5-nitropyridine

To a stirred solution of 3-bromo-6-ethyl-2-methoxy-5-nitropyridine (1.75 g, 6.703 mmol, 1.0 eq), and potassium, (5-chloro-2-fluorophenyl)trifluoroborate (2.06 g, 8.713 mmol, 1.3 eq) in dioxane (30.0 mL) was added a 2 M solution of sodium carbonate (1.42 g, 13.406 mmol, 2.0 eq) in water (6.7 mL) at rt. The resulting mixture was purged with nitrogen for 10 minute followed by addition of Pd(dppf)Cl₂.DCM (0.11 g, 0.134 mmol, 0.02 eq), again purged with nitrogen for 10 minute. The resulting mixture was heated at 120° C. for overnight. The progress of reaction was monitored by LCMS. The reaction mixture was diluted with EtOAc (100 mL), washed with water (50 mL), with brine (50 mL), dried over Na₂SO₄, concentrated and purified by combi flash chromatography [silica gel 100-200 mesh: elution 100% Hexane] to afford the desired compound 1.50 g, 72.46%) as yellow solid. LCMS: (M+1)⁺311.0

### Step-4: Synthesis of 5-(5-chloro-2-fluorophenyl)-2-ethyl-6-methoxypyridin-3-amine

To a stirred solution of 3-(5-chloro-2-fluorophenyl)-6-ethyl-2-methoxy-5-nitropyridine (1.50 g, 4.827 mmol, 1.0 eq) in EtOH (20.0 mL) and water (20.0 mL) was added Fe (2.16 g, 38.621 mmol, 8.0 eq) and NH₄Cl (2.60 g, 48.27 mmol, 10.0 eq) at rt. The resulting mixture was heated at 100° C. for 60 min. The progress of reaction was monitored by LCMS. The reaction mixture was filtered through celite the residue was washed with EtOH (50 mL) the filtrate was concentrated and the residue was dissolved in EtOAc (100 mL), washed with water (2×50 mL), dried over Na₂SO₄, and concentrated to afford the desired compound (1.30 g, 96.29%) as brown brown. LCMS: (M+1)⁺281.1

### Step-5: Synthesis of 5-amino-3-(5-chloro-2-fluorophenyl)-6-ethylpyridin-2(1H)-one

5-(5-chloro-2-fluorophenyl)-2-ethyl-6-methoxypyridin-3-amine (1.10 g, 3.918 mmol, 1.0 eq) was dissolved in aq. HBr (47%) (5.0 mL) at rt. The reaction mixture was stirred at 90° C. for 4 h. The progress of reaction was monitored by LCMS. The reaction mixture was basified with saturated aqueous solution of NaHCO₃(100 mL), extracted with EtOAc (2×100 mL). The combined organic layers were washed with water (50 mL), with brine (50 mL), dried over Na₂SO₄, and concentrated to afford the desired compound (0.90 g, 86.12%) as brown solid. LCMS: (M+1)⁺267.1.

### Step-6: Synthesis of N-(4-((5-(5-chloro-2-fluorophenyl)-2-ethyl-6-oxo-1,6-dihydropyridin-3-yl)amino)pyridin-2-yl)acetamide

To a stirred solution of compound 5-amino-3-(5-chloro-2-fluorophenyl)-6-ethylpyridin-2(1H)-one (0.20 g, 0.749 mmol, 1.0 eq) and N-(4-chloropyridin-2-yl)acetamide (0.14 g, 0.824 mmol, 1.1 eq) in dioxane (50 mL) was added Cs₂CO₃ (0.732 g, 2.247 mmol, 3.0 eq) at rt. The resulting mixture was purged with nitrogen for 10 min followed by addition of Pd₂(dba)₃ (0.082 g, 0.062 mmol, 0.12 eq) and xantphos (0.065 g, 0.112 mmol, 0.15 eq), again purged with nitrogen for 10 min. The reaction mixture was heated at 120° C. for 90 mi in microwave. The progress of reaction was monitored by LCMS. The reaction mixture was diluted with EtOAc (50 mL), washed with water (50 mL), with brine (50 mL), dried over Na₂SO₄, concentrated and purified by combi flash chromatography [silica gel 100-200 mesh: elution 0-10% MeOH in DCM] to afford the desired compound (0.095 g, 31.66%) as off white solid solid. LCMS: (M+1)⁺401.3; ¹H NMR (400 MHz, DMSO-d₆): δ 12.06 (brs, 1H) 10.11 (brs, 1H) 8.07 (brs, 1H) 7.82 (d, J=5.70 Hz, 1H) 7.63 (dd, J=6.36, 2.85 Hz, 1H) 7.38-7.48 (m, 2H) 7.28 (t, J=9.43 Hz, 1H) 6.23 (brs, 1H) 2.43-2.48 (m, 2H) 1.98-2.05 (s, 3H) 1.10 (t, J=7.45 Hz, 3H).

### Example-71: Synthesis of 5-((1,6-naphthyridin-4-yl)amino)-3-(5-chloro-2-fluorophenyl)-6-ethylpyridin-2(1H)-one (Compound 1.71)

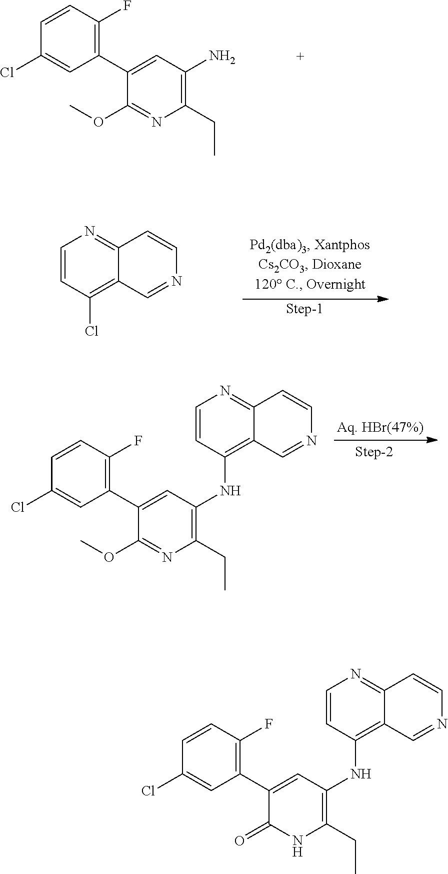 [SMILES: CCc1nc(OC)c(-c2cc(Cl)ccc2F)cc1N ; Clc1ccnc2ccncc12 ; CCc1nc(OC)c(-c2cc(Cl)ccc2F)cc1Nc1ccnc2ccncc12 ; CCc1[nH]c(=O)c(-c2cc(Cl)ccc2F)cc1Nc1ccnc2ccncc12]

### Step-1: Synthesis of N-(5-(5-chloro-2-fluorophenyl)-2-ethyl-6-methoxypyridin-3-yl)-1,6-naphthyridin-4-amine

To a stirred solution of compound 5-(5-chloro-2-fluorophenyl)-2-ethyl-6-methoxypyridin-3-amine (0.20 g, 0.712 mmol, 1.0 eq) and 4-chloro-1,6-naphthyridine (0.130 g, 0.783 mmol, 1.1 eq) in dioxane (10 mL) was added Cs₂CO₃ (0.70 g, 2.154 mmol, 3.0 eq) at rt. The resulting mixture was purged with nitrogen for 10 min followed by addition of Pd₂(dba)₃ (0.078 g, 0.085 mmol, 0.12 eq) and xantphos (0.062 g, 0.106 mmol, 0.15 eq), again purged with nitrogen for 10 min. The reaction mixture was heated at 120° C. for overnight. The progress of reaction was monitored by LCMS. The reaction mixture was diluted with EtOAc (100 mL), washed with water (50 mL), with brine (50 mL), dried over Na₂SO₄, concentrated and purified by combi flash chromatography [silica gel 100-200 mesh: elution 0-5% MeOH in DCM] to afford the desired compound N-(5-(5-chloro-2-fluorophenyl)-2-ethyl-6-methoxypyridin-3-yl)-1,6-naphthyridin-4-amine (50 mg, 17.18%) as brown solid. LCMS: (M+1)⁺409.2

### Step-2: Synthesis of 5-((1,6-naphthyridin-4-yl)amino)-3-(5-chloro-2-fluorophenyl)-6-ethylpyridin-2(1H)-one

N-(5-(5-chloro-2-fluorophenyl)-2-ethyl-6-methoxypyridin-3-yl)-1,6-naphthyridin-4-amine (0.05 g, 0.122 mmol, 1.0 eq) was dissolved in aq. HBr (47%) (2.0 mL) at rt. The reaction mixture was stirred at 70° C. for 4 h. The progress of reaction was monitored by LCMS. The reaction mixture was basified with saturated aqueous solution of NaHCO₃(100 mL), extracted with EtOAc (2×50 mL). The combined organic layers were washed with water (50 mL), with brine (50 mL), dried over Na₂SO₄, and concentrated and purified by combi flash chromatography [silica gel 100-200 mesh: elution 0-10% MeOH in DCM] to afford the desired compound 5-((1,6-naphthyridin-4-yl)amino)-3-(5-chloro-2-fluorophenyl)-6-ethylpyridin-2(1H)-one (0.010 g, 20.83%) as light yellow solid. LCMS: (M+1)⁺395.3; ¹H NMR (400 MHz, DMSO-d₆) δ 12.23 (brs, 1H) 9.76 (s, 1H) 9.10 (brs, 1H) 8.63 (d, J=6.14 Hz, 1H) 8.52 (d, J=5.26 Hz, 1H) 7.61-7.70 (m, 2H) 7.59 (s, 1H) 7.38-7.46 (m, 1H) 7.23-7.32 (m, 1H) 6.37 (d, J=5.70 Hz, 1H) 2.67 (q, J=7.45 Hz, 2H).1.12 (t, J=7.45 Hz, 3H).

### Example-72: Synthesis of 4-((5-(5-chloro-2-fluorophenyl)-2-ethyl-6-oxo-1,6-dihydropyridin-3-yl)amino)-N-methylnicotinamide (Compound 1.72)

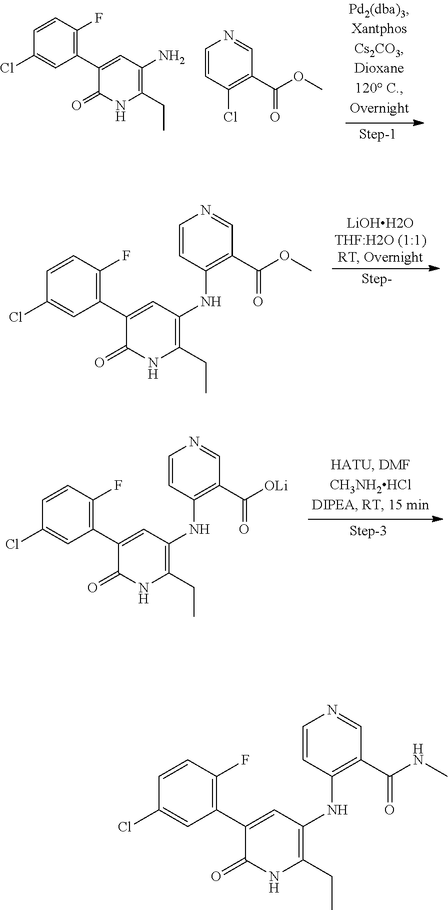

### Step-1: Synthesis of methyl 4-((5-(5-chloro-2-fluorophenyl)-2-ethyl-6-oxo-1,6-dihydropyridin-3-yl)amino)nicotinate

To a stirred solution of compound 5-amino-3-(5-chloro-2-fluorophenyl)-6-ethylpyridin-2(1H)-one (0.70 g, 2.624 mmol, 1.0 eq) and methyl 4-chloronicotinate (0.495 g, 2.887 mmol, 1.1 eq) in dioxane (30 mL) was added Cs₂CO₃ (2.56 g, 7.872 mmol, 3.0 eq) at rt. The resulting mixture was purged with nitrogen for 10 min followed by addition of Pd₂(dba)₃ (0.288 g, 0.315 mmol, 0.12 eq) and xantphos (0.227 g, 0.393 mmol, 0.15 eq), again purged with nitrogen for 10 min. The reaction mixture was heated at 120° C. for overnight. The progress of reaction was monitored by LCMS. The reaction mixture was diluted with EtOAc (1000 mL), washed with water (50 mL), with brine (50 mL), dried over Na₂SO₄, concentrated and purified by combi flash chromatography [silica gel 100-200 mesh: elution 0-5% MeOH in DCM] to afford the desired compound methyl 4-((5-(5-chloro-2-fluorophenyl)-2-ethyl-6-oxo-1,6-dihydropyridin-3-yl)amino)nicotinate (0.250 g, 23.76%) as light yellow solid. LCMS: (M+1)⁺402.2

### Step-2: Synthesis of lithium 4-((5-(5-chloro-2-fluorophenyl)-2-ethyl-6-oxo-1,6-dihydropyridin-3-yl)amino)nicotinate

To a stirred solution of methyl 4-((5-(5-chloro-2-fluorophenyl)-2-ethyl-6-oxo-1,6-dihydropyridin-3-yl)amino)nicotinate (0.250 g, 0.622 mmol, 1.0 eq) in THF (4 mL) and H₂O (1.0 mL) was added LiOH.H₂O (0.052 g, 1.244 mmol, 2.0 eq) at RT. The reaction mixture was stirred at RT for overnight. The progress of reaction was monitored by LCMS. The reaction mixture was concentrated to afford the desired compound lithium 4-((5-(5-chloro-2-fluorophenyl)-2-ethyl-6-oxo-1,6-dihydropyridin-3-yl)amino)nicotinate (0.24 g, 97.95%) as brown solid. LCMS: (M+1)⁺388.0

### Step-3: Synthesis of 4-((5-(5-chloro-2-fluorophenyl)-2-ethyl-6-oxo-1,6-dihydropyridin-3-yl)amino)-N-methylnicotinamide

To a stirred solution of lithium 4-((5-(5-chloro-2-fluorophenyl)-2-ethyl-6-oxo-1,6-dihydropyridin-3-yl)amino)nicotinate (0.250 g, 0.634 mmol, 1.0 eq) in DMF (2 mL) was added HATU (0.362 g, 0.952 mmol, 1.5 eq) at rt. The resulting mixture was stirred at rt for 5 minutes, followed by addition of CH₃NH₂.HCl (0.065 g, 0.952 mmol, 1.5 eq) and DIPEA (0.33 mL, 1.902 mmol, 3.0 eq) and stirred at rt for 15 min. Reaction mixture was diluted with water (20 mL) and extracted with ethyl acetate (2×20 mL). The combined organic extracts were washed with water (20 mL), with brine (20 mL) dried over Na₂SO₄ and concentrated under reduced and purified by combi flash chromatography [silica gel 100-200 mesh; elution 0-10% MeOH in DCM] to afford the desired compound 4-((5-(5-chloro-2-fluorophenyl)-2-ethyl-6-oxo-1,6-dihydropyridin-3-yl)amino)-N-methylnicotinamide (0.018 g, 07.50%) as brown solid. LCMS: (M+1)⁺401.3, ¹H NMR (400 MHz, DMSO-d₆) δ 12.14 (brs, 1H) 9.69 (brs, 1H) 8.67 (d, J=4.38 Hz, 1H) 8.63 (s, 1H) 8.15 (d, J=6.14 Hz, 1H) 7.64 (dd, J=6.14, 2.63 Hz, 1H) 7.40-7.51 (m, 2H) 7.23-7.35 (m, 2H) 6.50 (d, J=5.70 Hz, 1H) 2.79 (d, J=4.82 Hz, 3H) 2.45 (d, J=7.89 Hz, 2H) 1.10 (t, J=7.45 Hz, 3H).

### Example-73: Synthesis of 5-((1, 6-naphthyridin-4-yl)amino)-3-(5-chloro-2-fluorophenyl)-6-methoxy Pyridin-2(1H)-one (Compound 1.73)

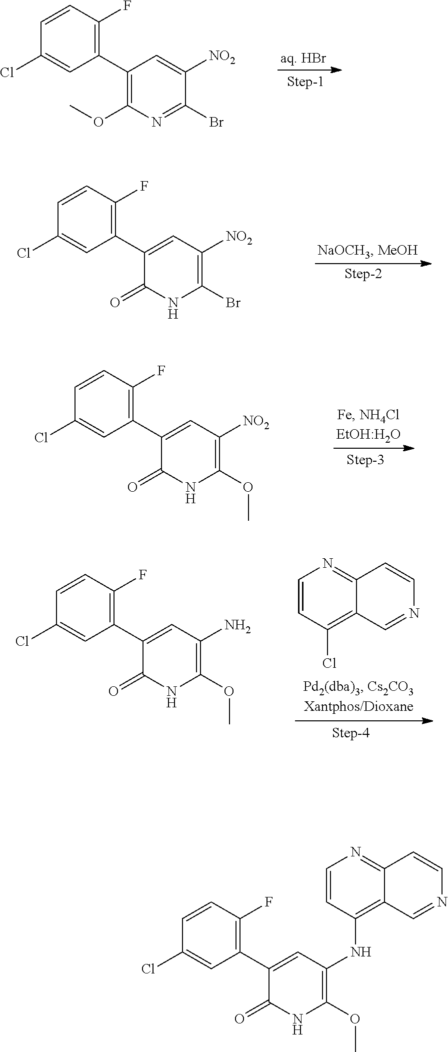 [SMILES: COc1nc(Br)c([N+](=O)[O-])cc1-c1cc(Cl)ccc1F ; O=c1[nH]c(Br)c([N+](=O)[O-])cc1-c1cc(Cl)ccc1F ; COc1[nH]c(=O)c(-c2cc(Cl)ccc2F)cc1[N+](=O)[O-] ; COc1[nH]c(=O)c(-c2cc(Cl)ccc2F)cc1N ; Clc1ccnc2ccncc12 ; COc1[nH]c(=O)c(-c2cc(Cl)ccc2F)cc1Nc1ccnc2ccncc12]

### Step-1: Synthesis of 6-bromo-3-(5-chloro-2-fluorophenyl)-5-nitropyridin-2(1H)-one

2-bromo-5-(5-chloro-2-fluorophenyl)-6-methoxy-3-nitropyridine (0.80 g, 2.212 mmol, 1.0 eq) and aq. HBr(47%) (3.0 mL) were stirred at 70° C. for 4 h. The progress of reaction was monitored by LCMS. The reaction mixture was neutralized with with saturated solution of NaHCO₃(100.0 mL), extracted with EtOAc (2×50 mL), the combined organic layers were washed with water (50.0 mL), with brine (50.0 mL), dried over Na₂SO₄, concentrated to afford the desired compound 6-bromo-3-(5-chloro-2-fluorophenyl)-5-nitropyridin-2(1H)-one (0.650 g, 84.41%) as yellow solid. LCMS: (M+1)⁺347.0

### Step-2: Synthesis of 3-(5-chloro-2-fluorophenyl)-6-methoxy-5-nitropyridin-2(1H)-one

To stirred solution of 6-bromo-3-(5-chloro-2-fluorophenyl)-5-nitropyridin-2(1H)-one (0.40 g, 1.151 mmol, 1.0 eq) in MeOH (3.0 mL) was added CH₃ONa (0.248 mg, 4.60 mmol, 4.0 eq) at rt. The resulting mixture was stirred at 70° C. for overnight. The progress of reaction was monitored by LCMS. The reaction mixture was concentrated and diluted with EtOAc (50.0 mL), washed with water (10 mL), with brine (10.0 mL), dried over Na₂SO₄, concentrated to afford the desired compound 3-(5-chloro-2-fluorophenyl)-6-methoxy-5-nitropyridin-2(1H)-one (0.340 mg, 98.83%) as yellow solid. LCMS: (M+1)⁺299.0

### Step-3: Synthesis of 5-amino-3-(5-chloro-2-fluorophenyl)-6-methoxypyridin-2(1H)-one

To a stirred solution of 3-(5-chloro-2-fluorophenyl)-6-methoxy-5-nitropyridin-2(1H)-one (0.550 g, 1.841 mmol, 1.0 eq) in EtOH (20.0 mL) and water (20.0 mL) was added Fe (0.822 g, 14.73 mmol, 8.0 eq) and a solution of NH₄Cl (0.985 g, 18.41 mmol, 10.0 eq) at RT. The resulting mixture was heated at 100° C. for 60 min. The progress of reaction was monitored by LCMS. The reaction mixture was filtered through celite the residue was washed with EtOH (50 mL) the filtrate was concentrated and the residue was dissolved in EtOAc (50 mL), washed with water (2×50 mL), dried over Na₂SO₄, and concentrated to afford the desired compound 5-amino-3-(5-chloro-2-fluorophenyl)-6-methoxypyridin-2(1H)-one (0.380 g, 76.76%) as brown solid. LCMS: (M+1)⁺269.0

### Step-4: Synthesis of 5-((1,6-naphthyridin-4-yl)amino)-3-(5-chloro-2-fluorophenyl)-6-methoxypyridin-2(1H)-one

To a stirred solution of 5-amino-3-(5-chloro-2-fluorophenyl)-6-methoxypyridin-2(1H)-one (0.20 g, 0.745 mmol, 1.0 eq) and N-(4-chloropyridin-2-yl)acetamide (0.135 g, 0.818 mmol, 1.1 eq) in dioxane (5.0 mL) was added Cs₂CO₃ (0.728 g, 2.235 mmol, 3.0 eq) at rt. The resulting mixture was purged with nitrogen for 10 min followed by addition of Pd₂(dba)₃ (0.082 g, 0.089 mmol, 0.12 eq) and xantphos (0.065 g, 0.111 mmol, 0.15 eq), again purged with nitrogen for 10 min. The reaction mixture was heated at 120° C. for overnight. The progress of reaction was monitored by LCMS. The reaction mixture was diluted with water (50.0 mL), extracted with EtOAc (2×50 mL), the combined organic layers were washed with water (50.0 mL), with brine (50.0 mL), dried over Na₂SO₄, concentrated and purified by combiflash [silica gel 100-200 mesh elution 0-10% MeOH in DCM] to afford the desired compound 5-((1,6-naphthyridin-4-yl)amino)-3-(5-chloro-2-fluorophenyl)-6-methoxypyridin-2(1H)-one (0.011 g, 3.70%) as yellow solid. LCMS: (M+1)⁺397.3, ¹H NMR (400 MHz, DMSO-d₆) δ 11.36 (brs, 1H) 9.76 (s, 1H) 9.21 (brs, 1H) 8.62 (d, J=6.14 Hz, 1H) 8.49 (d, J=4.82 Hz, 1H) 7.65-7.73 (m, 2H) 7.54 (dd, J=6.58, 2.63 Hz, 1H) 7.43-7.49 (m, 1H) 7.27-7.36 (m, 1H) 6.31 (d, J=5.26 Hz, 1H) 3.89 (s, 3H).

### Example-74: Synthesis 4-((5-(5-chloro-2-fluorophenyl)-2-cyclopropyl-6-oxo-1,6-dihydropyridin-3-yl)amino)nicotinamide (Compound 1.74)

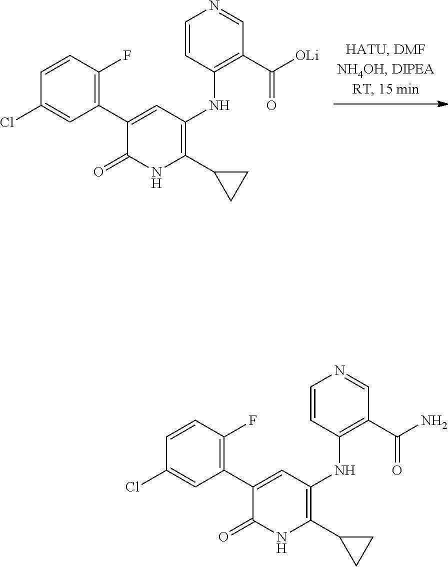

To a stirred solution of lithium 4-((5-(5-chloro-2-fluorophenyl)-2-cyclopropyl-6-oxo-1,6-dihydropyridin-3-yl)amino)nicotinate (0.015 g, 0.369 mmol, 1.0 eq) in DMF (2 mL) was added HATU (0.210 g, 0.554 mmol, 1.5 eq) at rt, followed by addition of NH₄OH (0.08 mL, 0.554 mmol, 1.5 eq) and DIPEA (0.192 mL, 1.107 mmol, 3.0 eq) and stirred at rt for 15 min. Reaction mixture was diluted with water (50 mL) and extracted with ethyl acetate (2×50 mL). The combined organic were washed with water (50 mL), with brine (50 mL) dried over Na₂SO₄ and concentrated under reduced and purified by combiflash [silica gel 100-200 mesh elution 0-10% MeOH in DCM] to afford the desired compound 4-((5-(5-chloro-2-fluorophenyl)-2-cyclopropyl-6-oxo-1,6-dihydropyridin-3-yl)amino)nicotinamide (0.0024 g, 16.32%) as brown solid. LCMS: (M+1)⁺399.3, ¹H NMR (400 MHz, DMSO-d₆) δ 11.31 (brs, 1H) 10.07 (brs, 1H) 8.71 (s, 1H) 8.10-8.27 (m, 2H) 7.60 (brs, 2H) 7.47 (brs, 2H) 7.23-7.30 (m, 1H) 6.51 (d, J=5.70 Hz, 1H) 1.90 (brs, 1H) 1.05 (brs, 2H) 0.91 (d, J=6.14 Hz, 2H).

### Example-75: Synthesis of N-(4-((5-(5-chloro-2-fluorophenyl)-2-methoxy-6-oxo-1,6-dihydropyridin-3-yl)amino)pyridin-2-yl)acetamide (Compound 1.75)

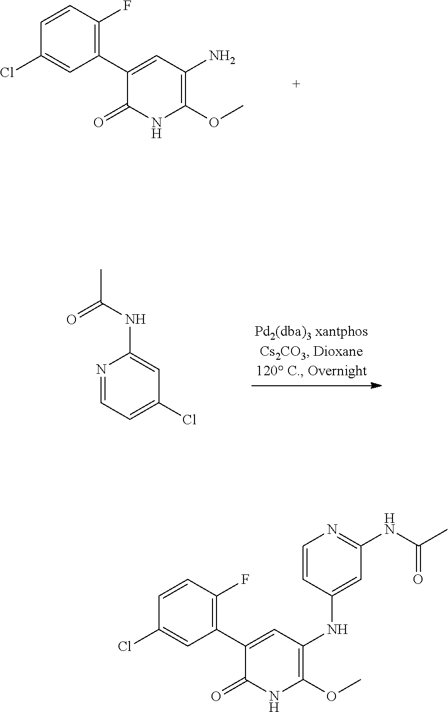 [SMILES: COc1[nH]c(=O)c(-c2cc(Cl)ccc2F)cc1N ; CC(=O)Nc1cc(Cl)ccn1 ; COc1[nH]c(=O)c(-c2cc(Cl)ccc2F)cc1Nc1ccnc(NC(C)=O)c1]

To a stirred solution of 5-amino-3-(5-chloro-2-fluorophenyl)-6-methoxypyridin-2(1H)-one (0.15 g, 0.55 mmol, 1.0 eq) and N-(4-chloropyridin-2-yl)acetamide (0.104 g, 0.61 mmol, 1.10 eq) in 1,4-Dioxane (10 mL) was added Cs₂CO₃ (0.545 g, 1.60 mmol, 3.0 eq). The reaction mixture was then purged vigorously with N2 atmosphere for 10 min. Pd₂(dba)₃ (0.061 g, 0.067 mmol, 0.12 eq) and Xantphos (0.048 g, 0.083 mmol, 0.15 eq) was added to the reaction mass followed by heating it at 130° C. for overnight. The progress of reaction was monitored by TLC & LCMS. The reaction mixture was diluted with EtOAc (2×25 mL) and water (25 mL). The combined organic layers were washed with brine (25 mL), dried over Na₂SO₄, concentrated and purified by RP-HPLC to afford the desired compound N-(4-((5-(5-chloro-2-fluorophenyl)-6-hydroxy-2-methoxypyridin-3-yl)amino)pyridin-2-yl)acetamide (0.070 g, 31.25%) as white fluffy solid. LCMS: (M+1)⁺403.3, ¹H NMR (400 MHz, DMSO-d₆) δ 11.06 (brs, 1H), 10.12 (brs, 2H), 8.14 (brs, 2H), 7.82 (d, J=5.70 Hz, 1H), 7.53 (d, J=10.96 Hz, 1H), 7.42 (brs, 1H), 7.26-7.38 (m, 1H), 6.32 (brs, 1H), 3.89 (s, 3H) 2.03 (s, 3H).

### Example-76: Synthesis of 5-((1H-pyrrolo[2,3-b]pyridin-4-yl)amino)-3-(5-chloro-2-fluorophenyl)-6-methoxypyridin-2(1H)-one (Compound 1.76)

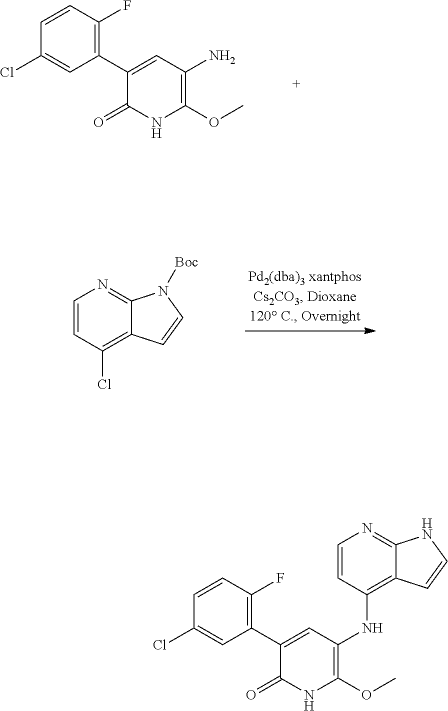 [SMILES: COc1[nH]c(=O)c(-c2cc(Cl)ccc2F)cc1N ; CC(C)(C)OC(=O)n1ccc2c(Cl)ccnc21 ; COc1[nH]c(=O)c(-c2cc(Cl)ccc2F)cc1Nc1ccnc2[nH]ccc12]

To a stirred solution of 5-amino-3-(5-chloro-2-fluorophenyl)-6-methoxypyridin-2(1H)-one (0.15 g, 0.55 mmol, 1.0 eq) and tert-butyl 4-chloro-1H-pyrrolo[2,3-b]pyridine-1-carboxylate (0.155 g, 0.61 mmol, 1.10 eq) in 1,4-Dioxane (10 mL) was added Cs₂CO₃ (0.545 g, 1.60 mmol, 3.0 eq). The reaction mixture was then purged vigorously with N2 atmosphere for 10 min. Pd₂(dba)₃ (0.061 g, 0.067 mmol, 0.12 eq) and Xantphos (0.048 g, 0.083 mmol, 0.15 eq) was added to the reaction mass followed by heating it at 130° C. for overnight. The progress of reaction was monitored by TLC & LCMS. The reaction mixture was diluted with EtOAc (2×25 mL) and water (25 mL). The combined organic layers were washed with brine (25 mL), dried over Na₂SO₄, concentrated and purified by RP-HPLC to afford the desired compound 5-((1H-pyrrolo[2,3-b]pyridin-4-yl)amino)-3-(5-chloro-2-fluorophenyl)-6-methoxypyridin-2-ol (0.013 g, 6%) as white fluffy solid. LCMS: (M+1)⁺385.3; ¹H NMR (400 MHz, DMSO-d₆) δ 11.30 (brs, 1H), 8.03 (brs, 1H), 7.78 (d, J=5.70 Hz, 1H), 7.48-7.64 (m, 1H), 7.43 (dd, J=8.33, 3.51 Hz, 1H), 7.31 (t, J=9.21 Hz, 1H), 7.12 (brs, 1H), 6.52 (brs, 1H), 6.03 (d, J=5.26 Hz, 1H), 3.85 (m, 3H).

### Example-77: Synthesis of 3-(5-chloro-2-fluorophenyl)-6-cyclopropyl-5-((2-((5-methylthiazol-2-yl)amino)pyridin-4-yl)amino)pyridin-2(1H)-one (Compound 1.77)

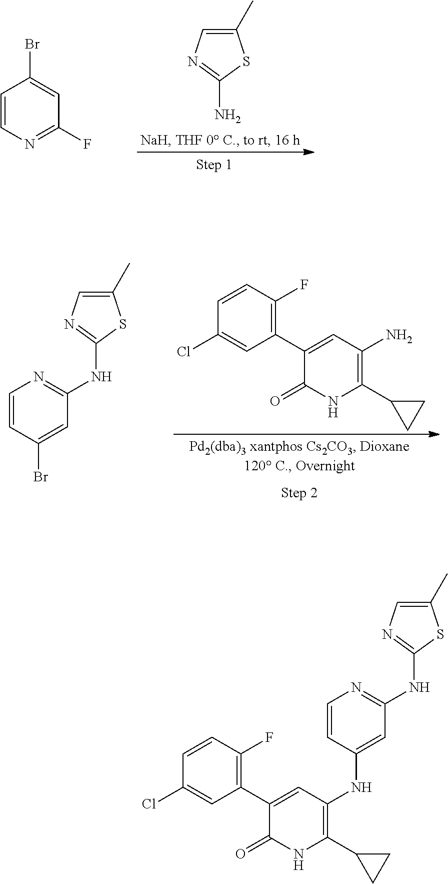

### Step 1: Synthesis of N-(4-bromopyridin-2-yl)-5-methylthiazol-2-amine

To a stirred solution of 5-methylthiazol-2-amine (0.50 g, 4.30 mmol, 1.0 eq), in THF (10 mL) was added NaH (350 mg, 8.60 mmol, 2.0 eq) portion-wise at 0° C. Reaction mass was then allowed to stir at rt for 15 min. 4-bromo-2-fluoropyridine (770 mg, 4.30 mmol, 1.0 eq) was then added drop-wise dissolved in THF (2 mL). Reaction mass was then allowed to stir at rt for 16 h. After completion of the reaction, monitored by TLC and LCMS, reaction mass was then quenched with water (25 mL) followed by extraction with EtOAc (2×25 mL) and water (25 mL). The combined organic layers were washed with brine (25 mL), dried over Na₂SO₄, concentrated and purified by combi-flash to get the desired product (100 mg, 13%) as off-white solid. LCMS: (M+1)⁺271.8

### Step 2: Synthesis of 3-(5-chloro-2-fluorophenyl)-6-cyclopropyl-5-((2-((5-methylthiazol-2-yl)amino)pyridin-4-yl)amino)pyridin-2(1H)-one

N-(4-bromopyridin-2-yl)-5-methylthiazol-2-amine (0.10 g, 0.37 mmol, 1.0 eq) and 5-amino-3-(5-chloro-2-fluorophenyl)-6-cyclopropyl pyridine-2(1H)-one (0.113 g, 0.40 mmol, 1.10 eq) in 1,4-Dioxane (8 mL) was added Cs₂CO₃ (0.362 g, 1.11 mmol, 3.0 eq). The reaction mixture was then purged vigorously with N₂ atmosphere for 10 min. Pd₂(dba)₃ (0.040 g, 0.044 mmol, 0.12 eq) and Xantphos (0.032 g, 0.055 mmol, 0.15 eq) was added to the reaction mass followed by heating it at 130° C. for overnight. The progress of reaction was monitored by TLC & LCMS. The reaction mixture was diluted with EtOAc (2×25 mL) and water (25 mL). The combined organic layers were washed with brine (25 mL), dried over Na₂SO₄, concentrated and purified by RP-HPLC to afford the desired compound 3-(5-chloro-2-fluorophenyl)-6-cyclopropyl-5-((2-((5-methylthiazol-2-yl)amino) pyridin-4-yl)amino)pyridin-2(1H)-one (0.008 g, 4.50%) as white fluffy solid. LCMS: (M+1)⁺468.3, ¹H NMR (400 MHz, DMSO-d₆) δ 11.40 (brs, 1H) 9.80 (brs, 1H) 7.93 (brs, 1H) 7.58 (dd, J=6.14, 2.63 Hz, 1H) 7.42-7.54 (m, 2H) 7.31 (t, J=9.21 Hz, 1H) 7.12 (brs, 1H) 6.45 (brs, 2H) 6.04 (brs, 1H) 2.33 (brs, 3H) 1.96 (brs, 1H) 1.09 (d, J=2.63 Hz, 2H) 0.87-0.99 (m, 2H).

### Example-78: Synthesis of 4-((5-(5-chloro-2-fluorophenyl)-2-isopropyl-6-oxo-1,6-dihydropyridin-3-yl)amino)-5,7-dihydro-6H-pyrrolo[2,3-d]pyrimidin-6-one. (Compound 1.78)

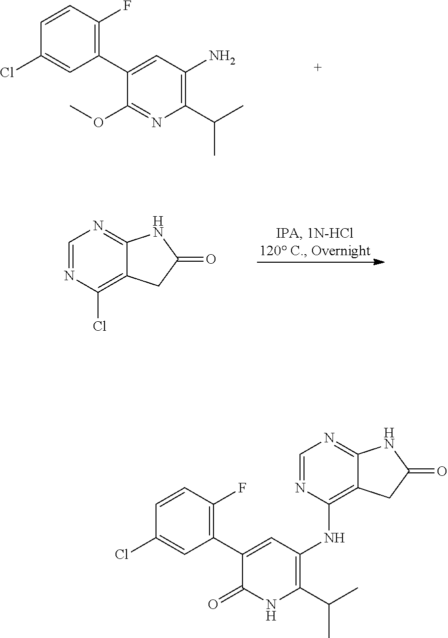 [SMILES: COc1nc(C(C)C)c(N)cc1-c1cc(Cl)ccc1F ; O=C1Cc2c(Cl)ncnc2N1 ; CC(C)c1[nH]c(=O)c(-c2cc(Cl)ccc2F)cc1Nc1ncnc2c1CC(=O)N2]

To a stirred solution of 5-(5-chloro-2-fluorophenyl)-2-isopropyl-6-methoxypyridin-3-amine (0.30 g, 1.018 mmol, 1.0 eq) and 4-chloro-5,7-dihydro-6H-pyrrolo[2,3-d]pyrimidin-6-one (0.172 g, 1.018 mmol, 1.0 eq) in isopropyl alcohol (0.5 mL) was added 1N—HCl (0.509 mL, 0.509 mmol, 0.5 eq) at RT. The reaction mixture was heated at 120° C. for overnight. The progress of reaction was monitored by LCMS. The reaction mixture was neutralized with saturated solution of NaHCO₃(100 mL), extracted with EtOAc (2×50 mL). The combined organic layers were washed with brine (50 mL), dried over Na₂SO₄, concentrated and purified by combi-flash chromatography [silica gel 100-200 mesh: elution 0-10% MeOH in DCM] to afford the desired compound 4-((5-(5-chloro-2-fluorophenyl)-2-isopropyl-6-oxo-1,6-dihydropyridin-3-yl)amino)-5,7-dihydro-6H-pyrrolo[2,3-d]pyrimidin-6-one (0.015 g, 3.57%) as brown solid. LCMS: (M+1)⁺414.4, ¹H NMR (400 MHz, DMSO-d₆) δ 12.06 (brs, 1H) 8.09 (s, 1H) 7.51-7.59 (m, 1H) 7.39-7.51 (m, 2H) 7.31 (t, J=9.43 Hz, 1H) 6.83 (s, 2H) 3.52 (s, 1H) 3.44 (s, 1H) 2.69-2.81 (m, 1H) 1.17 (d, J=6.58 Hz, 3H) 1.18 (d, J=6.58 Hz, 3H).

### Example-79: Synthesis of N-(4-((2-isopropyl-6-oxo-5-(thiophen-3-yl)-1,6-dihydropyridin-3-yl)amino)pyridin-2-yl)acetamide (Compound 1.79)

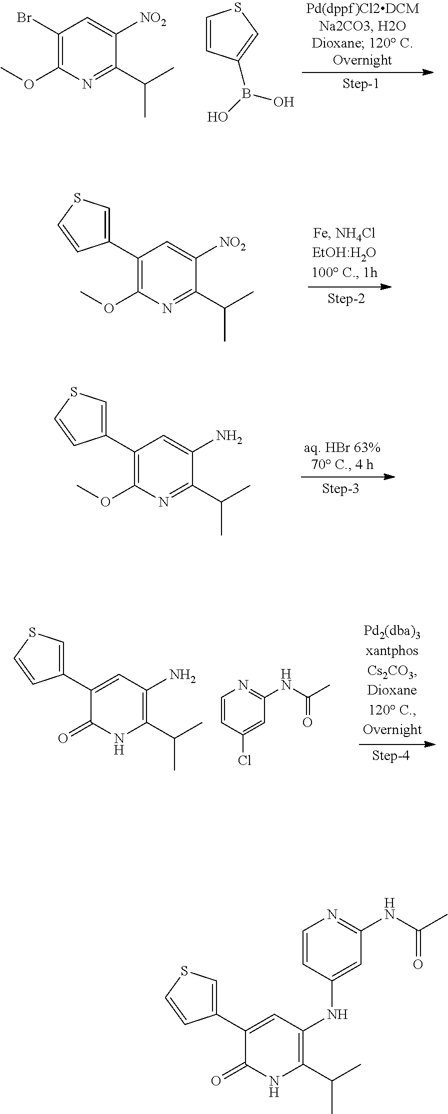

### Step-1: Synthesis 2-isopropyl-6-methoxy-3-nitro-5-(thiophen-3-yl)pyridine

To a stirred solution of 3-bromo-6-isopropyl-2-methoxy-5-nitropyridine (1.8 g, 6.543 mmol, 1.0 eq), and thiophen-3-ylboronic acid (1.0 g, 7.851 mmol, 1.2 eq) in dioxane (30.0 mL) was added a 2 M solution of sodium carbonate (1.4 g, 13.08 mmol, 2.0 eq) in water (6.5 mL) at RT. The resulting mixture was purged with nitrogen for 10 minute followed by addition of Pd(dppf)Cl₂.DCM (0.11 g, 0.130 mmol, 0.02 eq), again purged with nitrogen for 10 minute. The resulting mixture was heated at 120° C. for overnight. The progress of reaction was monitored by LCMS. The reaction mixture was diluted with EtOAc (100 mL), washed with water (50 mL), with brine (50 mL), dried over Na₂SO₄, concentrated and purified by combi flash chromatography [silica gel 100-200 mesh: elution 100% Hexane] to afford the desired compound 2-isopropyl-6-methoxy-3-nitro-5-(thiophen-3-yl)pyridine (1.60 g, 88.88%) as yellow solid. LCMS: (M+1)⁺278.9

### Step-2: Synthesis of 2-isopropyl-6-methoxy-5-(thiophen-3-yl)pyridin-3-amine

To a stirred solution of 2-isopropyl-6-methoxy-3-nitro-5-(thiophen-3-yl)pyridine (1.60 g, 5.79 mmol, 1.0 eq) in EtOH (25.0 mL) and water (25.0 mL) was added Fe (2.58 g, 46.329 mmol, 8.0 eq) and NH₄Cl (3.10 g, 57.90 mmol, 10.0 eq) at RT. The resulting mixture was heated at 100° C. for 60 min. The progress of reaction was monitored by LCMS. The reaction mixture was filtered through celite the residue was washed with EtOH (50 mL) the filtrate was concentrated and the residue was dissolved in EtOAc (100 mL), washed with water (2×50 mL), dried over Na₂SO₄, and concentrated to afford the desired compound 2-isopropyl-6-methoxy-5-(thiophen-3-yl)pyridin-3-amine (1.4 g, 98.59%) as brown solid. LCMS: (M+1)⁺249.2

### Step-3: Synthesis of 5-amino-6-isopropyl-3-(thiophen-3-yl)pyridin-2(1H)-one

2-isopropyl-6-methoxy-5-(thiophen-3-yl)pyridin-3-amine (1.40 g, 5.637 mmol, 1.0 eq) was dissolved in aq. HBr (63%) (5.0 mL) at RT. The reaction mixture was stirred at 70° C. for 4 h. The progress of reaction was monitored by LCMS. The reaction mixture was basified with saturated aqueous solution of NaHCO₃(100 mL), extracted with EtOAc (2×100 mL). The combined organic layers were washed with water (50 mL), with brine (50 mL), dried over Na₂SO₄, and concentrated to afford the desired compound 5-amino-6-isopropyl-3-(thiophen-3-yl)pyridin-2(1H)-one (0.90 g, 67.16%) as brown solid. LCMS: (M+1)⁺235.1

### Step-4: Synthesis of N-(4-((2-isopropyl-6-oxo-5-(thiophen-3-yl)-1,6-dihydropyridin-3-yl)amino)pyridin-2-yl)acetamide

To a stirred solution of compound 5-amino-6-isopropyl-3-(thiophen-3-yl)pyridin-2(1H)-one (0.20 g, 0.853 mmol, 1.0 eq) and N-(4-chloropyridin-2-yl)acetamide (0.16 g, 0.938 mmol, 1.1 eq) in dioxane (5.0 mL) was added Cs₂CO₃ (0.835 g, 2.559 mmol, 3.0 eq) at RT. The resulting mixture was purged with nitrogen for 10 min followed by addition of Pd₂(dba)₃ (0.094 g, 0.102 mmol, 0.12 eq) and xantphos (0.074 g, 0.127 mmol, 0.15 eq), again purged with nitrogen for 10 min. The reaction mixture was heated at 120° C. for overnight. The progress of reaction was monitored by LCMS. The reaction mixture was diluted with EtOAc (50 mL), washed with water (50 mL), with brine (50 mL), dried over Na₂SO₄, concentrated and purified by reverse phase purification to afford the desired compound N-(4-((2-isopropyl-6-oxo-5-(thiophen-3-yl)-1,6-dihydropyridin-3-yl)amino)pyridin-2-yl)acetamide (0.120 mg, 38.21%) as off white solid. LCMS: (M+1)⁺369.4, ¹H NMR (400 MHz, DMSO-d₆) δ 11.70 (brs, 1H) 10.07 (s, 1H) 8.35 (brs, 1H) 8.03 (s, 1H) 7.81 (d, J=5.70 Hz, 1H) 7.61-7.73 (m, 2H) 7.45-7.55 (m, 1H) 7.35 (brs, 1H) 6.21 (brs, 1H) 2.97-3.10 (m, 1H) 1.99 (s, 3H) 1.19 (d, J=7.02 Hz, 6H).

### Example-80: Synthesis of 5-((1,6-naphthyridin-4-yl)amino)-6-isopropyl-3-(thiophen-3-yl)pyridin-2(1H)-one (Compound 1.80)

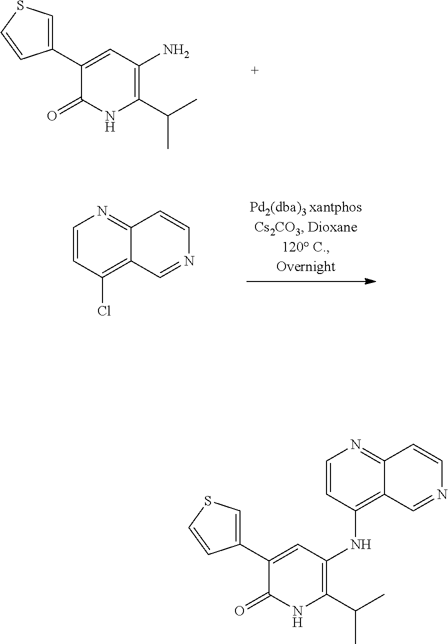 [SMILES: CC(C)c1[nH]c(=O)c(-c2ccsc2)cc1N ; Clc1ccnc2ccncc12 ; CC(C)c1[nH]c(=O)c(-c2ccsc2)cc1Nc1ccnc2ccncc12]

To a stirred solution of compound 5-amino-6-isopropyl-3-(thiophen-3-yl)pyridin-2(1H)-one (0.160 g, 0.682 mmol, 1.0 eq) and 4-chloro-1,6-naphthyridine (0.124 g, 0.751 mmol, 1.1 eq) in dioxane (5.0 mL) was added Cs₂CO₃ (0.670 g, 2.046 mmol, 3.0 eq) at RT. The resulting mixture was purged with nitrogen for 10 min followed by addition of Pd₂(dba)₃ (0.075 g, 0.082 mmol, 0.12 eq) and xantphos (0.060 g, 0.102 mmol, 0.15 eq), again purged with nitrogen for 10 min. The reaction mixture was heated at 120° C. for overnight. The progress of reaction was monitored by LCMS. The reaction mixture was diluted with EtOAc (50 mL), washed with water (50 mL), with brine (50 mL), dried over Na₂SO₄, concentrated and purified by reverse phase purification to afford the desired compound 5-((1,6-naphthyridin-4-yl)amino)-6-isopropyl-3-(thiophen-3-yl)pyridin-2(1H)-one (0.048 mg, 32.65%) as yellow solid. LCMS: (M+1)⁺363.4, ¹H NMR (400 MHz, DMSO-d₆) δ 11.87 (brs, 1H) 9.78 (s, 1H) 9.13 (brs, 1H) 8.64 (d, J=5.70 Hz, 1H) 8.49 (d, J=5.26 Hz, 1H) 8.41 (brs, 1H) 7.89 (s, 1H) 7.67-7.74 (m, 2H) 7.51 (dd, J=5.26, 3.07 Hz, 1H) 6.32 (s, 1H) 3.01-3.11 (m, 1H) 1.21 (d, J=7.02 Hz, 6H).

### Example-81: Synthesis of N-(4-((5-(3-chlorophenyl)-2-isopropyl-6-oxo-1,6-dihydropyridin-3-yl)amino)pyridin-2-yl)acetamide (Compound 1.81)

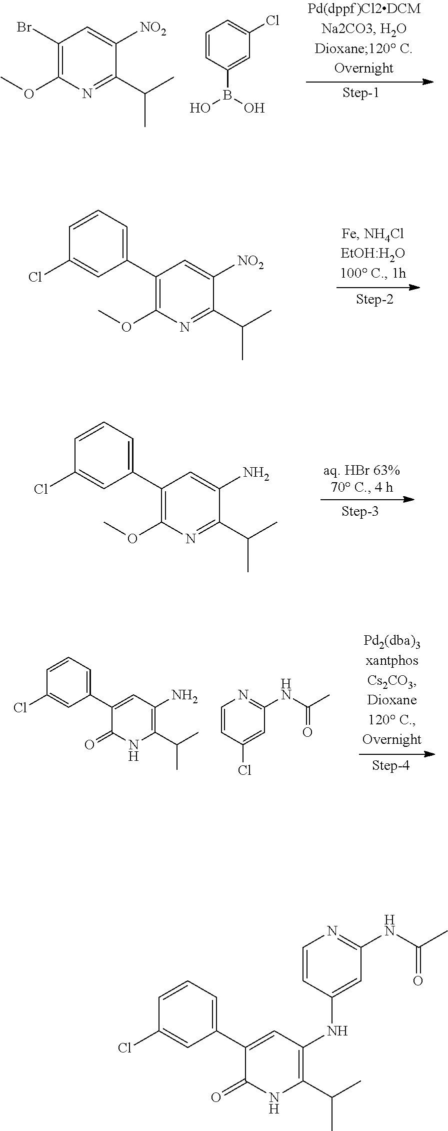

### Step-1: Synthesis 3-(3-chlorophenyl)-6-isopropyl-2-methoxy-5-nitropyridine

To a stirred solution of 3-bromo-6-isopropyl-2-methoxy-5-nitropyridine (1.0 g, 3.635 mmol, 1.0 eq), and (3-chlorophenyl)boronic acid (0.68 g, 4.362 mmol, 1.2 eq) in dioxane (20.0 mL) was added a 2 M solution of sodium carbonate (0.77 g, 7.27 mmol, 2.0 eq) in water (3.6 mL) at RT. The resulting mixture was purged with nitrogen for 10 minute followed by addition of Pd(dppf)Cl₂.DCM (0.060 g, 0.073 mmol, 0.02 eq), again purged with nitrogen for 10 minute. The resulting mixture was heated at 120° C. for overnight. The progress of reaction was monitored by LCMS. The reaction mixture was diluted with EtOAc (100 mL), washed with water (50 mL), with brine (50 mL), dried over Na₂SO₄, concentrated and purified by combi flash chromatography [silica gel 100-200 mesh: elution 100% Hexane] to afford the desired compound 3-(3-chlorophenyl)-6-isopropyl-2-methoxy-5-nitropyridine (1.00 g, 89.92%) as yellow solid. LCMS: (M+1)⁺307.0

### Step-2: Synthesis of 5-(3-chlorophenyl)-2-isopropyl-6-methoxypyridin-3-amine

To a stirred solution of 3-(3-chlorophenyl)-6-isopropyl-2-methoxy-5-nitropyridine (1.0 g, 3.26 mmol, 1.0 eq) in EtOH (20.0 mL) and water (20.0 mL) was added Fe (1.46 g, 26.08 mmol, 8.0 eq) and NH₄Cl (1.74 g, 32.60 mmol, 10.0 eq) at RT. The resulting mixture was heated at 100° C. for 60 min. The progress of reaction was monitored by LCMS. The reaction mixture was filtered through celite the residue was washed with EtOH (50 mL) the filtrate was concentrated and the residue was dissolved in EtOAc (100 mL), washed with water (2×50 mL), dried over Na₂SO₄, and concentrated to afford the desired compound 5-(3-chlorophenyl)-2-isopropyl-6-methoxypyridin-3-amine (0.80 g, 88.20%) as brown solid. LCMS: (M+1)⁺277.1

### Step-3: Synthesis of 5-amino-3-(3-chlorophenyl)-6-isopropylpyridin-2(1H)-one

5-(3-chlorophenyl)-2-isopropyl-6-methoxypyridin-3-amine (0.8 g, 2.89 mmol, 1.0 eq) was dissolved in aq. HBr (63%) (2.0 mL) at RT. The reaction mixture was stirred at 70° C. for 4 h. The progress of reaction was monitored by LCMS. The reaction mixture was basified with saturated aqueous solution of NaHCO₃(100 mL), extracted with EtOAc (2×100 mL). The combined organic layers were washed with water (50 mL), with brine (50 mL), dried over Na₂SO₄, and concentrated to afford the desired compound 5-amino-3-(3-chlorophenyl)-6-isopropylpyridin-2(1H)-one (0.72 g, 94.73%) as brown solid. LCMS: (M+1)⁺263.0

### Step-4: Synthesis of N-(4-((5-(3-chlorophenyl)-2-isopropyl-6-oxo-1,6-dihydropyridin-3-yl)amino)pyridin-2-yl)acetamide

To a stirred solution of compound 5-amino-3-(3-chlorophenyl)-6-isopropylpyridin-2(1H)-one (0.20 g, 0.761 mmol, 1.0 eq) and N-(4-chloropyridin-2-yl)acetamide (0.143 g, 0.837 mmol, 1.1 eq) in dioxane (5.0 mL) was added Cs₂CO₃ (0.745 g, 2.283 mmol, 3.0 eq) at RT. The resulting mixture was purged with nitrogen for 10 min followed by addition of Pd₂(dba)₃ (0.084 g, 0.092 mmol, 0.12 eq) and xantphos (0.072 g, 0.125 mmol, 0.15 eq), again purged with nitrogen for 10 min. The reaction mixture was heated at 120° C. for overnight. The progress of reaction was monitored by LCMS. The reaction mixture was diluted with EtOAc (50 mL), washed with water (50 mL), with brine (50 mL), dried over Na₂SO₄, concentrated and purified by reverse phase purification to afford the desired compound N-(4-((5-(3-chlorophenyl)-2-isopropyl-6-oxo-1,6-dihydropyridin-3-yl)amino)pyridin-2-yl)acetamide (0.1350 mg, 44.70%) as off white solid. LCMS: (M+1)⁺397.4, ¹H NMR (400 MHz, DMSO-d₆) δ 13.20 (brs, 1H) 11.94 (brs, 1H) 11.07 (brs, 1H) 9.27 (brs, 1H) 7.83-7.96 (m, 2H) 7.72 (d, J=7.45 Hz, 1H) 7.63 (s, 1H) 7.35-7.42 (m, 2H) 6.58 (brs, 2H) 2.92-3.07 (m, 2H) 2.10 (s, 3H) 1.21 (d, J=7.02 Hz, 6H).

### Example-82: Synthesis of 4-((5-(4-fluoro-3-methylphenyl)-2-isopropyl-6-oxo-1,6-dihydro pyridine-3-yl) amino)-5, 7-dihydro-6H-pyrrolo[2, 3-d]pyrimidin-6-one (Compound 1.82)

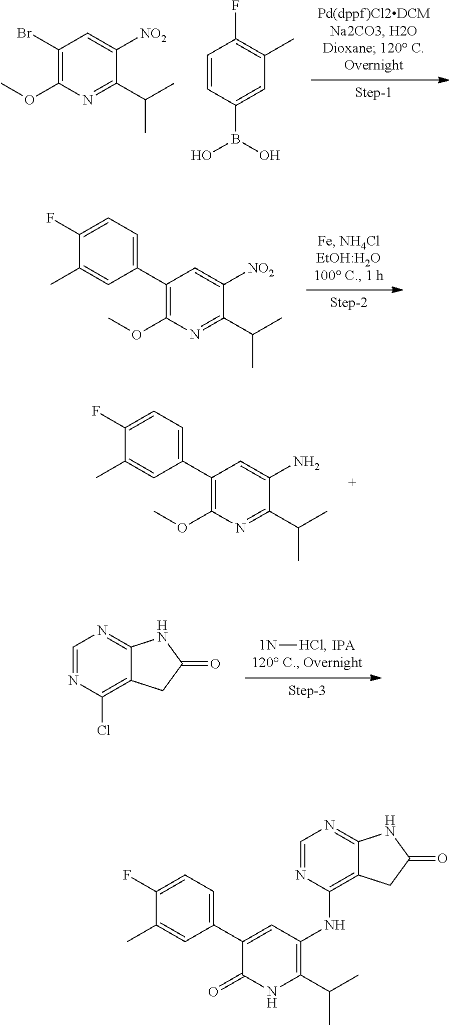

### Step-1: Synthesis 3-(4-fluoro-3-methylphenyl)-6-isopropyl-2-methoxy-5-nitropyridine

To a stirred solution of 3-bromo-6-isopropyl-2-methoxy-5-nitropyridine (1.0 g, 3.635 mmol, 1.0 eq), and (4-fluoro-3-methylphenyl)boronic acid (0.68 g, 4.362 mmol, 1.2 eq) in dioxane (20.0 mL) was added a 2 M solution of sodium carbonate (0.77 g, 7.27 mmol, 2.0 eq) in water (3.6 mL) at RT. The resulting mixture was purged with nitrogen for 10 minute followed by addition of Pd(dppf)Cl₂.DCM (0.060 g, 0.073 mmol, 0.02 eq), again purged with nitrogen for 10 minute. The resulting mixture was heated at 120° C. for overnight. The progress of reaction was monitored by LCMS. The reaction mixture was diluted with EtOAc (100 mL), washed with water (50 mL), with brine (50 mL), dried over Na₂SO₄, concentrated and purified by combi flash chromatography [silica gel 100-200 mesh: elution 100% Hexane] to afford the desired compound 3-(4-fluoro-3-methylphenyl)-6-isopropyl-2-methoxy-5-nitropyridine (1.00 g 90.90%) as yellow solid. LCMS: (M+1)⁺305.2

### Step-2: Synthesis of 5-(4-fluoro-3-methylphenyl)-2-isopropyl-6-methoxypyridin-3-amine

To a stirred solution of 3-(4-fluoro-3-methylphenyl)-6-isopropyl-2-methoxy-5-nitropyridine (1.0 g, 3.286 mmol, 1.0 eq) in EtOH (20.0 mL) and water (20.0 mL) was added Fe (1.47 g, 26.288 mmol, 8.0 eq) and NH₄Cl (1.75 g, 32.86 mmol, 10.0 eq) at RT. The resulting mixture was heated at 100° C. for 60 min. The progress of reaction was monitored by LCMS. The reaction mixture was filtered through celite the residue was washed with EtOH (50 mL) the filtrate was concentrated and the residue was dissolved in EtOAc (100 mL), washed with water (2×50 mL), dried over Na₂SO₄, and concentrated to afford the desired compound 5-(4-fluoro-3-methylphenyl)-2-isopropyl-6-methoxypyridin-3-amine (0.80 g, 88.88%) as brown liquid. LCMS: (M+1)⁺275.1

### Step-3: Synthesis of 4-((5-(4-fluoro-3-methylphenyl)-2-isopropyl-6-oxo-1,6-dihydropyridin-3-yl)amino)-5,7-dihydro-6H-pyrrolo[2,3-d]pyrimidin-6-one

To a stirred solution of 5-(4-fluoro-3-methylphenyl)-2-isopropyl-6-methoxypyridin-3-amine (0.20 g, 0.729 mmol, 1.0 eq) and 4-chloro-5,7-dihydro-6H-pyrrolo[2,3-d]pyrimidin-6-one (0.125 g, 0.729 mmol, 1.0 eq) in isopropyl alcohol (0.36 mL) was added 1N—HCl (0.36 mL, 0.0.36 mmol, 0.5 eq) at RT. The reaction mixture was heated at 120° C. for overnight. The progress of reaction was monitored by LCMS. The reaction mixture was neutralized with saturated solution of NaHCO₃(100 mL), extracted with EtOAc (2×50 mL). The combined organic layers were washed with brine (50 mL), dried over Na₂SO₄, concentrated and purified by SFC to afford the desired compound 4-((5-(4-fluoro-3-methylphenyl)-2-isopropyl-6-oxo-1,6-dihydropyridin-3-yl)amino)-5,7-dihydro-6H-pyrrolo[2,3-d]pyrimidin-6-one (0.015 g, 3.57%) as brown solid. LCMS: (M+1)⁺394.4, ¹H NMR (400 MHz, DMSO-d₆) δ 11.89 (brs, 1H) 8.08 (s, 1H) 7.59 (d, J=7.45 Hz, 2H) 7.53 (s, 1H) 7.08-7.17 (m, 1H) 6.82 (s, 1H) 3.58 (s, 1H) 3.53 (s, 1H) 2.67-2.77 (m, 1H) 2.24 (s, 3H) 1.06-1.21 (m, 6H).

### Example-83: Synthesis of N-(4-((5-(4-fluoro-3-methylphenyl)-2-isopropyl-6-oxo-1, 6-dihydropyridin-3-yl)amino)pyridin-2-yl)acetamide (Compound 1.83)

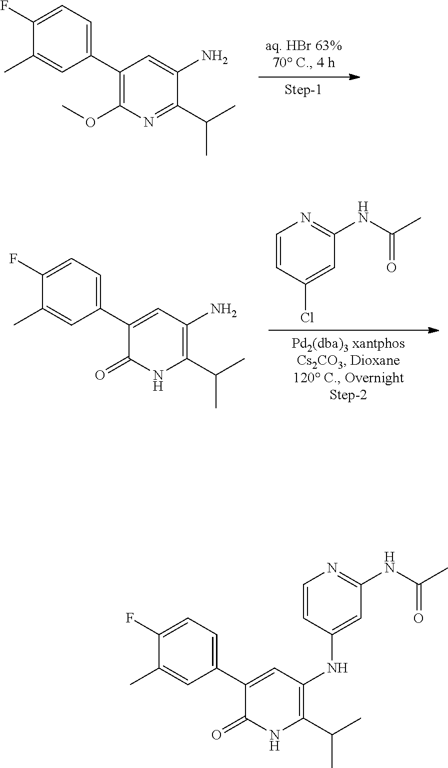

### Step-1: Synthesis of 5-amino-3-(4-fluoro-3-methylphenyl)-6-isopropylpyridin-2(1H)-one

5-(4-fluoro-3-methylphenyl)-2-isopropyl-6-methoxypyridin-3-amine (0.50 g, 1.822 mmol, 1.0 eq) was dissolved in aq. HBr (63%) (3.0 mL) at RT. The reaction mixture was stirred at 70° C. for 4 h. The progress of reaction was monitored by LCMS. The reaction mixture was basified with saturated aqueous solution of NaHCO₃(100 mL), extracted with EtOAc (2×100 mL). The combined organic layers were washed with water (50 mL), with brine (50 mL), dried over Na₂SO₄, and concentrated to afford the desired compound 5-amino-3-(4-fluoro-3-methylphenyl)-6-isopropylpyridin-2(1H)-one (0.45 g, 94.93%) as brown solid. LCMS: (M+1)⁺261.0

### Step-2: Synthesis of N-(4-((5-(4-fluoro-3-methylphenyl)-2-isopropyl-6-oxo-1,6-dihydropyridin-3-yl)amino)pyridin-2-yl)acetamide

To a stirred solution of compound 5-amino-3-(4-fluoro-3-methylphenyl)-6-isopropylpyridin-2(1H)-one (0.20 g, 0.768 mmol, 1.0 eq) and N-(4-chloropyridin-2-yl)acetamide (0.144 g, 0.845 mmol, 1.1 eq) in dioxane (10.0 mL) was added Cs₂CO₃ (0.750 g, 2.304 mmol, 3.0 eq) at RT. The resulting mixture was purged with nitrogen for 10 min followed by addition of Pd₂(dba)₃ (0.085 g, 0.092 mmol, 0.12 eq) and xantphos (0.066 g, 0.115 mmol, 0.15 eq), again purged with nitrogen for 10 min. The reaction mixture was heated at 120° C. for overnight. The progress of reaction was monitored by LCMS. The reaction mixture was diluted with EtOAc (50 mL), washed with water (50 mL), with brine (50 mL), dried over Na₂SO₄, concentrated and purified by combiflash chromatography [silica gel 100-200; elution 0-5% MeOH in DCM to afford the desired compound N-(4-((5-(4-fluoro-3-methylphenyl)-2-isopropyl-6-oxo-1,6-dihydropyridin-3-yl)amino)pyridin-2-yl)acetamide (0.079 mg, 26.07%) as off white solid. LCMS: (M+1)⁺395.5, ¹H NMR (400 MHz, DMSO-d₆) δ 11.68 (brs, 1H) 10.15 (brs, 1H) 8.12 (brs, 1H) 7.82 (d, J=5.70 Hz, 1H) 7.64 (d, J=6.58 Hz, 2H) 7.42 (s, 1H) 7.32 (brs, 1H) 7.12 (t, J=8.99 Hz, 1H) 6.25 (brs, 1H) 3.06 (dt, J=13.81, 7.13 Hz, 1H) 2.24 (s, 3H) 2.01 (s, 3H) 1.03-1.23 (m, 6H).

### Example-84: Synthesis of 5-((1H-pyrazolo[3,4-b]pyridin-4-yl)amino)-3-(3-chloro-4-fluoro phenyl)-6-isopropylpyridin-2(1H)-one (Compound 1.84)

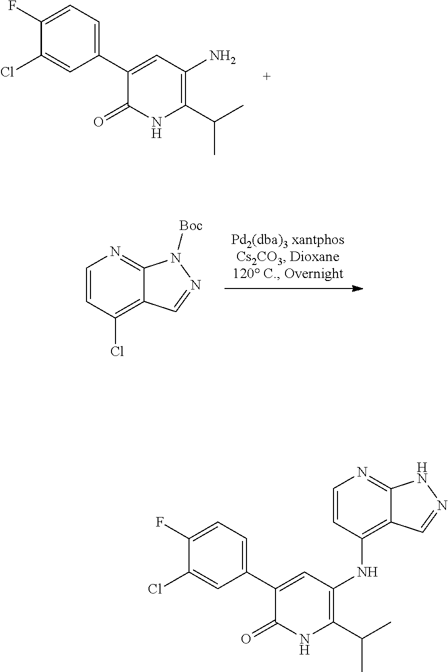 [SMILES: CC(C)c1[nH]c(=O)c(-c2ccc(F)c(Cl)c2)cc1N ; CC(C)(C)OC(=O)n1ncc2c(Cl)ccnc21 ; "Pd2(dba)3 xantphosCs2CO3, Dioxane120° C., Overnight" ; CC(C)c1[nH]c(=O)c(-c2ccc(F)c(Cl)c2)cc1Nc1ccnc2[nH]ncc12]

To a stirred solution of 5-amino-3-(3-chloro-4-fluorophenyl)-6-isopropylpyridin-2(1H)-one (0.20 g, 0.71 mmol, 1.0 eq) and tert-butyl 4-chloro-1H-pyrazolo[3,4-b]pyridine-1-carboxylate (0.20 g, 0.78 mmol, 1.10 eq) in 1,4-Dioxane (10 mL) was added Cs₂CO₃ (0.70 g, 2.10 mmol, 3.0 eq). The reaction mixture was then purged vigorously with N2 atmosphere for 10 min. Pd₂(dba)₃ (0.078 g, 0.085 mmol, 0.12 eq) and Xantphos (0.060 g, 0.010 mmol, 0.15 eq) was added to the reaction mass followed by heating it at 120° C. for overnight. The progress of reaction was monitored by TLC & LCMS. The reaction mixture was diluted with EtOAc (2×25 mL) and water (25 mL). The combined organic layers were washed with brine (25 mL), dried over Na₂SO₄, concentrated and purified by RP-HPLC to afford the desired compound 5-((1H-pyrazolo[3,4-b]pyridin-4-yl)amino)-3-(3-chloro-4-fluorophenyl)-6-isopropylpyridin-2(1H)-one (0.005 g, 1.76%) as white fluffy solid. LCMS: (M+1)⁺398.4; ¹H NMR (400 MHz, DMSO-d₆) δ 13.16 (s, 1H) 11.91 (s, 1H) 8.66 (s, 1H) 8.08 (d, J=7.45 Hz, 1H) 7.99 (d, J=5.26 Hz, 1H) 7.79 (brs, 1H) 7.71 (s, 1H) 7.40 (t, J=9.21 Hz, 1H) 5.99 (s, 1H) 3.05-3.18 (m, 1H) 1.20 (d, J=7.02 Hz, 6H).

### Example-85: Synthesis of 4-((5-(3-chloro-4-fluorophenyl)-2-isopropyl-6-oxo-1,6-dihydropyridin-3-yl)amino)-5, 7-dihydro-6H-pyrrolo[2,3-d]pyrimidin-6-one (Compound 1.85)

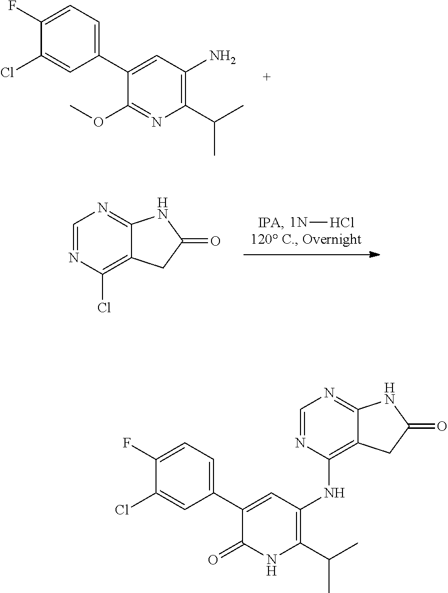

To a stirred solution of 5-(3-chloro-4-fluorophenyl)-2-isopropyl-6-methoxypyridin-3-amine (0.20 g, 0.678 mmol, 1.0 eq) and 4-chloro-5,7-dihydro-6H-pyrrolo[2,3-d]pyrimidin-6-one (0.115 g, 0.678 mmol, 1.0 eq) in isopropyl alcohol (0.34 mL) was added 1N—HCl (0.34 mL, 0.34 mmol, 0.5 eq) at RT. The reaction mixture was heated at 120° C. for overnight. The progress of reaction was monitored by LCMS. The reaction mixture was neutralized with saturated solution of NaHCO₃(100 mL), extracted with EtOAc (2×50 mL). The combined organic layers were washed with brine (50 mL), dried over Na₂SO₄, concentrated and purified by reverse phase to afford the desired compound 4-((5-(3-chloro-4-fluorophenyl)-2-isopropyl-6-oxo-1,6-dihydropyridin-3-yl)amino)-5,7-dihydro-6H-pyrrolo[2,3-d]pyrimidin-6-one (0.004 g, 1.42%) as white solid. LCMS: (M+1)⁺414.4; ¹H NMR (400 MHz, DMSO-d₆) δ 11.96 (s, 1H) 8.09 (s, 1H) 8.00 (d, J=4.82 Hz, 1H) 7.74-7.80 (m, 1H) 7.71 (s, 1H) 7.44 (t, J=9.21 Hz, 1H) 6.83 (s, 2H) 3.60 (s, 1H) 3.54 (s, 1H) 3.44 (s, 1H) 2.73-2.82 (m, 1H) 1.06-1.32 (m, 6H).

### Example-86: Synthesis of 5-((7H-pyrrolo[2,3-d]pyrimidin-4-yl)amino)-3-(5-chloro-2-fluorophenyl)-6-isopropylpyridin-2(1H)-one (Compound 1.86)

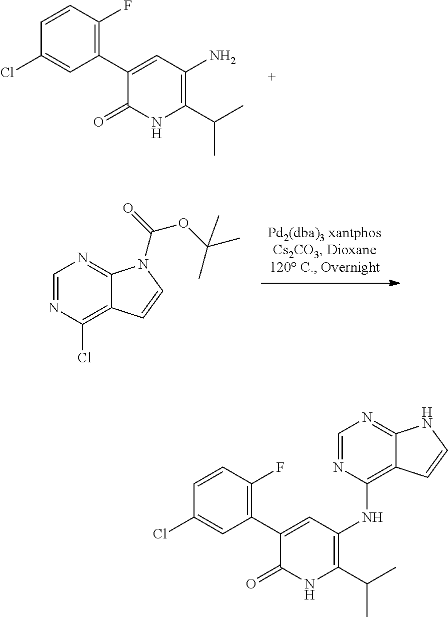 [SMILES: CC(C)c1[nH]c(=O)c(-c2cc(Cl)ccc2F)cc1N ; CC(C)(C)OC(=O)n1ccc2c(Cl)ncnc21 ; CC(C)c1[nH]c(=O)c(-c2cc(Cl)ccc2F)cc1Nc1ncnc2[nH]ccc12]

To a stirred solution of compound 5-amino-3-(5-chloro-2-fluorophenyl)-6-isopropylpyridin-2(1H)-one (0.20 g, 0.714 mmol, 1.0 eq) and tert-butyl 4-chloro-7H-pyrrolo[2,3-d]pyrimidine-7-carboxylate (0.220 g, 0.856 mmol, 1.2 eq) in dioxane (20.0 mL) was added Cs₂CO₃ (0.700 g, 2.142 mmol, 3.0 eq) at RT. The resulting mixture was purged with nitrogen for 10 min followed by addition of Pd₂(dba)₃ (0.078 g, 0.086 mmol, 0.12 eq) and xantphos (0.062 g, 0.107 mmol, 0.15 eq), again purged with nitrogen for 10 min. The reaction mixture was heated at 120° C. for overnight. The progress of reaction was monitored by LCMS. The reaction mixture was diluted with EtOAc (50 mL), washed with water (50 mL), with brine (50 mL), dried over Na₂SO₄, concentrated and purified by reverse phase purification to afford the desired compound 5-((7H-pyrrolo[2,3-d]pyrimidin-4-yl)amino)-3-(5-chloro-2-fluorophenyl)-6-isopropylpyridin-2(1H)-one (0.004 mg, 1.41%) as white solid. LCMS: (M+1)⁺398.4; ¹H NMR (400 MHz, DMSO-d₆) δ 11.80 (s, 1H) 11.64 (brs, 1H) 8.77 (brs, 1H) 8.16 (s, 1H) 8.07 (s, 1H) 7.60 (d, J=3.95 Hz, 1H) 7.49 (s, 1H) 7.43 (dd, J=8.33, 3.51 Hz, 1H) 7.26-7.35 (m, 1H) 7.14 (brs, 1H) 3.08-3.16 (m, 1H) 1.03-1.28 (m, 6H).

### Example-87: Synthesis of 5-((1,6-naphthyridin-4-yl)amino)-3-(3-chloro-4-fluorophenyl)-6-ethylpyridin-2(1H)-one (Compound 1.87)

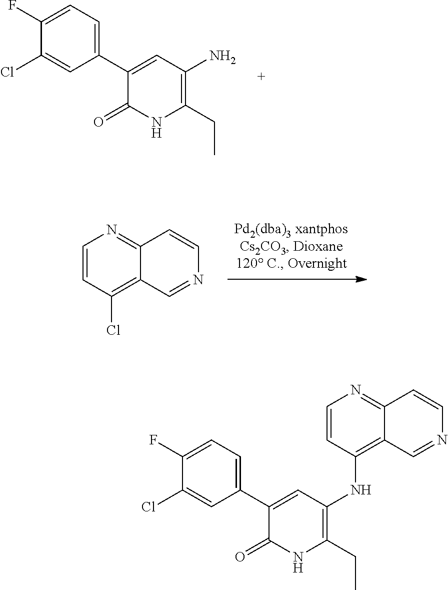 [SMILES: CCc1[nH]c(=O)c(-c2ccc(F)c(Cl)c2)cc1N ; Clc1ccnc2ccncc12 ; CCc1[nH]c(=O)c(-c2ccc(F)c(Cl)c2)cc1Nc1ccnc2ccncc12]

To a stirred solution of compound 5-amino-3-(3-chloro-4-fluorophenyl)-6-ethylpyridin-2(1H)-one (0.200 g, 0.749 mmol, 1.0 eq) and 4-chloro-1,6-naphthyridine (0.135 g, 0.824 mmol, 1.1 eq) in dioxane (5.0 mL) was added Cs₂CO₃ (0.735 g, 2.247 mmol, 3.0 eq) at RT. The resulting mixture was purged with nitrogen for 10 min followed by addition of Pd₂(dba)₃ (0.082 g, 0.084 mmol, 0.12 eq) and xantphos (0.065 g, 0.114 mmol, 0.15 eq), again purged with nitrogen for 10 min. The reaction mixture was heated at 120° C. for overnight. The progress of reaction was monitored by LCMS. The reaction mixture was diluted with EtOAc (50 mL), washed with water (50 mL), with brine (50 mL), dried over Na₂SO₄, concentrated and purified by reverse phase purification to afford the desired compound 5-((1,6-naphthyridin-4-yl)amino)-3-(3-chloro-4-fluorophenyl)-6-ethylpyridin-2(1H)-one (0.066 mg, 22.29%) as yellow solid. LCMS: (M+1)⁺395.4; ¹H NMR (400 MHz, DMSO-d₆) δ 12.20 (brs, 1H) 9.77 (s, 1H) 9.09 (brs, 1H) 8.63 (d, J=5.70 Hz, 1H) 8.50 (d, J=5.26 Hz, 1H) 8.11 (dd, J=7.45, 2.19 Hz, 1H) 7.77-7.87 (m, 2H) 7.68 (d, J=5.70 Hz, 1H) 7.41 (t, J=9.21 Hz, 1H) 6.36 (d, J=5.26 Hz, 1H), 2.50 (q, 2H) 1.11 (t, J=7.45 Hz, 3H).

### Example-88: Synthesis of N-(4-((5-(3-chloro-4-fluorophenyl)-2-ethyl-6-oxo-1,6-dihydropyridin-3-yl)amino)pyridin-2-yl)acetamide (Compound 1.88)

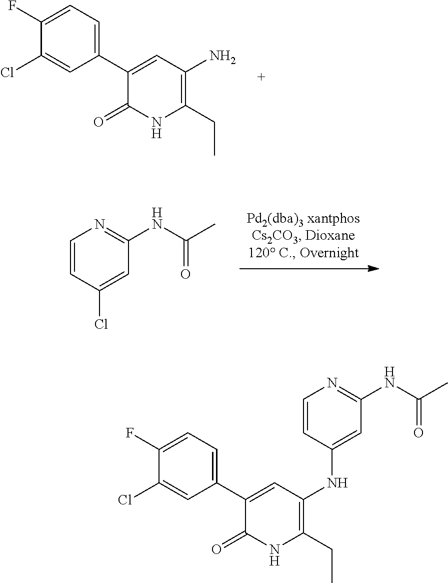 [SMILES: CCc1[nH]c(=O)c(-c2ccc(F)c(Cl)c2)cc1N ; CC(=O)Nc1cc(Cl)ccn1 ; CCc1[nH]c(=O)c(-c2ccc(F)c(Cl)c2)cc1Nc1ccnc(NC(C)=O)c1]

To a stirred solution of compound 5-amino-3-(3-chloro-4-fluorophenyl)-6-ethylpyridin-2(1H)-one (0.20 g, 0.749 mmol, 1.0 eq) and N-(4-chloropyridin-2-yl)acetamide (0.140 g, 0.824 mmol, 1.1 eq) in dioxane (5.0 mL) was added Cs₂CO₃ (0.735 g, 2.247 mmol, 3.0 eq) at RT. The resulting mixture was purged with nitrogen for 10 min followed by addition of Pd₂(dba)₃ (0.082 g, 0.082 mmol, 0.12 eq) and xantphos (0.065 g, 0.114 mmol, 0.15 eq), again purged with nitrogen for 10 min. The reaction mixture was heated at 120° C. for overnight. The progress of reaction was monitored by LCMS. The reaction mixture was diluted with EtOAc (50 mL), washed with water (50 mL), with brine (50 mL), dried over Na₂SO₄, concentrated and purified by reverse phase purification to afford the desired compound N-(4-((5-(3-chloro-4-fluorophenyl)-2-ethyl-6-oxo-1,6-dihydropyridin-3-yl)amino)pyridin-2-yl)acetamide (0.13 mg, 43.47%) as yellow solid. LCMS: (M+1)⁺401.4; ¹H NMR (400 MHz, DMSO-d₆) δ 12.05 (brs, 1H) 10.10 (s, 1H) 8.16 (brs, 1H) 8.03-8.12 (m, 2H) 7.73-7.89 (m, 2H) 7.60 (s, 1H) 7.33-7.48 (m, 2H) 6.24 (brs, 1H) 2.40-2.47 (m, 2H) 2.01 (s, 3H) 1.09 (t, J=7.45 Hz, 3H).

### Example-89: Synthesis of 5-((1,6-naphthyridin-4-yl)amino)-3-(4-fluoro-3-methylphenyl)-6-isopropylpyridin-2(1H)-one (Compound 1.89)

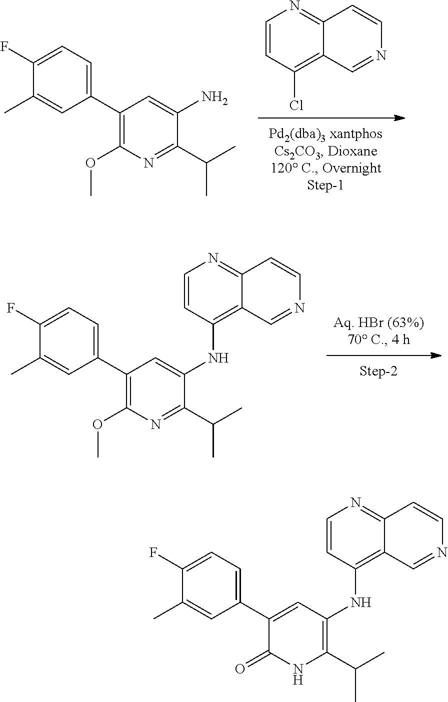 [SMILES: COc1nc(C(C)C)c(N)cc1-c1ccc(F)c(C)c1 ; Clc1ccnc2ccncc12 ; COc1nc(C(C)C)c(Nc2ccnc3ccncc23)cc1-c1ccc(F)c(C)c1 ; Cc1cc(-c2cc(Nc3ccnc4ccncc34)c(C(C)C)[nH]c2=O)ccc1F]

### Step-1: Synthesis of N-(5-(4-fluoro-3-methylphenyl)-2-isopropyl-6-methoxypyridin-3-yl)-1,6-naphthyridin-4-amine

To a stirred solution of compound 5-(4-fluoro-3-methylphenyl)-2-isopropyl-6-methoxypyridin-3-amine (0.200 g, 0.729 mmol, 1.0 eq) and 4-chloro-1,6-naphthyridine (0.132 g, 0.802 mmol, 1.1 eq) in dioxane (5.0 mL) was added Cs₂CO₃ (0.712 g, 2.187 mmol, 3.0 eq) at RT. The resulting mixture was purged with nitrogen for 10 min followed by addition of Pd₂(dba)₃ (0.080 g, 0.087 mmol, 0.12 eq) and xantphos (0.063 g, 0.109 mmol, 0.15 eq), again purged with nitrogen for 10 min. The reaction mixture was heated at 120° C. for overnight. The progress of reaction was monitored by LCMS. The reaction mixture was diluted with EtOAc (50 mL), washed with water (50 mL), with brine (50 mL), dried over Na₂SO₄, concentrated and purified by combi flash [silica gel 100-200 mesh elution 0-5% meOH in DCM] to afford the desired compound N-(5-(4-fluoro-3-methylphenyl)-2-isopropyl-6-methoxypyridin-3-yl)-1,6-naphthyridin-4-amine (0.160 g, 54.42%) as brown solid. LCMS: (M+1)⁺403.2.

### Step-2: Synthesis of 5-((1,6-naphthyridin-4-yl)amino)-3-(4-fluoro-3-methylphenyl)-6-isopropylpyridin-2(1H)-one

N-(5-(4-fluoro-3-methylphenyl)-2-isopropyl-6-methoxypyridin-3-yl)-1,6-naphthyridin-4-amine (0.160 g, 0.398 mmol, 1.0 eq) was dissolved in aq. HBr (63%) (2.0 mL) at RT. The reaction mixture was stirred at 70° C. for 4 h. The progress of reaction was monitored by LCMS. The reaction mixture was basified with saturated aqueous solution of NaHCO₃(100 mL), extracted with EtOAc (2×100 mL). The combined organic layers were washed with water (50 mL), with brine (50 mL), dried over Na₂SO₄, and concentrated to afford the desired compound 5-((1,6-naphthyridin-4-yl)amino)-3-(4-fluoro-3-methylphenyl)-6-isopropylpyridin-2(1H)-one (0.144 g, 93.50%) as yellow solid. LCMS: (M+1)⁺389.4; ¹H NMR (400 MHz, DMSO-d₆) δ 11.85 (brs, 1H) 9.79 (s, 1H) 9.27 (brs, 1H) 8.66 (d, J=5.70 Hz, 1H) 8.50 (d, J=5.70 Hz, 1H) 7.55-7.74 (m, 4H) 7.13 (t, J=9.43 Hz, 1H) 6.37 (d, J=5.70 Hz, 1H) 3.00-3.13 (m, 1H) 2.24 (s, 3H) 1.22 (d, J=7.02 Hz, 6H).

### Example-90: Synthesis of 5-((1H-pyrrolo[2,3-b]pyridin-4-yl)amino)-3-(3-chloro-4-fluorophenyl)-6-ethylpyridin-2(1H)-one (Compound 1.90)

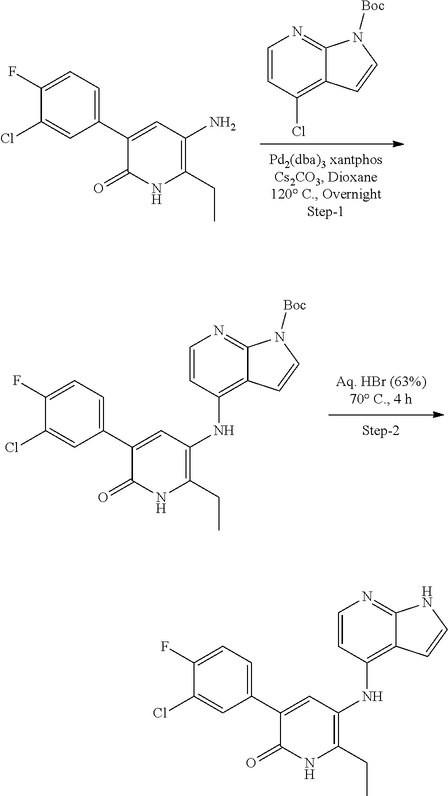 [SMILES: CCc1[nH]c(=O)c(-c2ccc(F)c(Cl)c2)cc1N ; CC(C)(C)OC(=O)n1ccc2c(Cl)ccnc21 ; CCc1[nH]c(=O)c(-c2ccc(F)c(Cl)c2)cc1Nc1ccnc2c1ccn2C(=O)OC(C)(C)C ; CCc1[nH]c(=O)c(-c2ccc(F)c(Cl)c2)cc1Nc1ccnc2[nH]ccc12]

### Step-1: Synthesis of tert-butyl 4-((5-(3-chloro-4-fluorophenyl)-2-ethyl-6-oxo-1,6-dihydropyridin-3-yl)amino)-1H-pyrrolo[2,3-b]pyridine-1-carboxylate

To a stirred solution of compound 5-amino-3-(3-chloro-4-fluorophenyl)-6-ethylpyridin-2(1H)-one (0.180 g, 0.674 mmol, 1.0 eq) and tert-butyl 4-chloro-1H-pyrrolo[2,3-b]pyridine-1-carboxylate (0.190 g, 0.742 mmol, 1.1 eq) in dioxane (2.0 mL) was added Cs₂CO₃ (0.660 g, 2.022 mmol, 3.0 eq) at RT. The resulting mixture was purged with nitrogen for 10 min followed by addition of Pd₂(dba)₃ (0.075 g, 0.080 mmol, 0.12 eq) and xantphos (0.060 g, 0.101 mmol, 0.15 eq), again purged with nitrogen for 10 min. The reaction mixture was heated at 120° C. for overnight. The progress of reaction was monitored by LCMS. The reaction mixture was diluted with EtOAc (50 mL), washed with water (50 mL), with brine (50 mL), dried over Na₂SO₄, concentrated and purified by combi flash [silica gel 100-200 mesh elution 0-5% meOH in DCM] to afford the desired compound tert-butyl 4-((5-(3-chloro-4-fluorophenyl)-2-ethyl-6-oxo-1,6-dihydropyridin-3-yl)amino)-1H-pyrrolo[2,3-b]pyridine-1-carboxylate (0.250 g, 69.06%) as brown solid. LCMS: (M+1)⁺483.1

### Step-2: Synthesis of 5-((1H-pyrrolo[2,3-b]pyridin-4-yl)amino)-3-(3-chloro-4-fluorophenyl)-6-ethylpyridin-2(1H)-one

tert-butyl 4-((5-(3-chloro-4-fluorophenyl)-2-ethyl-6-oxo-1,6-dihydropyridin-3-yl)amino)-1H-pyrrolo [2,3-b]pyridine-1-carboxylate (0.250 g, 0.517 mmol, 1.0 eq) was dissolved in 4.0M HCl in dioxane (2.0 mL) at RT. The reaction mixture was stirred at RT for overnight. The progress of reaction was monitored by LCMS. The reaction mixture was basified with saturated aqueous solution of NaHCO₃(100 mL), extracted with EtOAc (2×100 mL). The combined organic layers were washed with water (50 mL), with brine (50 mL), dried over Na₂SO₄, concentrated and purified by combi flash [silica gel 100-200 mesh elution 0-5% MeOH in DCM] to afford the desired compound 5-((1H-pyrrolo [2,3-b]pyridin-4-yl)amino)-3-(3-chloro-4-fluorophenyl)-6-ethylpyridin-2(1H)-one (0.06 g, 30.30%) as brown solid. LCMS: (M+1)⁺383.4; ¹H NMR (400 MHz, DMSO-d₆) δ 12.09 (brs, 1H) 11.27 (brs, 1H) 8.09 (dd, J=7.45, 2.19 Hz, 1H) 8.03 (s, 1H) 7.73-7.81 (m, 2H) 7.68 (s, 1H) 7.39 (t, J=8.99 Hz, 1H) 7.13 (d, J=3.07 Hz, 1H) 6.49 (d, J=3.07 Hz, 1H) 5.96 (d, J=5.26 Hz, 1H) 3.38 (q, J=7.02 Hz, 2H) 1.04-1.16 (m, 3H).

It is understood that compounds from the Table-2 are synthesized using the General Synthetic Schemes 1-7 or according to the experimental details as exemplified in Examples 1-90 using the appropriate starting materials and reagents.

### Biological Examples

### Example-B1: In Vitro Kinase Assay (ALK5 IC₅₀ Determination)

In Vitro Kinase Assay—

Compounds of the invention were screened in an in vitro kinase assay against ALK 5, a members of the TGF-β family of Ser/Thr kinases. Standard kinase testing conditions and techniques were employed. IC₅₀ values of compounds against TGF-ß kinase was determined by LanthaScreen™ Terbium Labeled TR-FRET assay. Kinase assays were performed in 1X kinase buffer (# PV6135, Invitrogen, Life Technologies Grand Island, N.Y.) where total reaction volume was 10 μL in low-volume 384-well plates (#4511, Corning). Serially diluted compounds (3-fold) were incubated with TGF-β kinase (6 nM) for 10 min, following which a mixture of ATP (10 μM) (# A1852, Sigma, St-Louis, Mo.) and fluorescent-PolyGT substrate (200 nM); Cat # PV3610, Invitrogen, Life Technologies Grand Island, N.Y.) was added and incubated in dark at room temperature for 1 h. After 1 h, 10 μL stop solution containing Terbium labeled antibody (4 nM; Cat # PV3529, Invitrogen, Life Technologies Grand Island, N.Y.) and EDTA (# E5134, Sigma, St-Louis, Mo.; 20 mM) in TR-FRET dilution buffer (Cat # PV3574, Invitrogen, Life Technologies Grand Island, N.Y.) was added. Readings were taken in a Synergy Neo Plate reader (BioTek, Winooski) at single excitation of 340 nm and Dual emission at 495 nm and 520 nm respectively.

The % activity of test samples was calculated as (Sample-Min)×100/(Max −Min). [Max: DMSO control, complete reaction with enzyme with DMSO and Min: No enzyme with DMSO]. Percent inhibition (100-% activity) was fitted to the “four-parameter logistic model” in XLfit for determination of IC₅₀ values. IC₅₀ values are presented in Table-3.

TABLE 3

| | Enzyme Activity | | Enzyme Activity |
|---|---|---|---|
| Comp. No. | ALK 5 IC₅₀ (uM) | Comp. No. | ALK 5 IC₅₀ (uM) |
| 1.1 | C | 1.2 | D |
| 1.3 | C | 1.4 | C |
| 1.5 | A | 1.6 | B |
| 1.7 | D | 1.8 | D |
| 1.9 | C | 1.10 | A |
| 1.11 | D | 1.12 | A |
| 1.13 | C | 1.14 | D |
| 1.15 | D | 1.16 | D |
| 1.17 | C | 1.18 | A |
| 1.19 | C | 1.20 | D |
| 1.21 | B | 1.22 | C |
| 1.23 | D | 1.24 | D |
| 1.25 | D | 1.26 | D |
| 1.27 | D | 1.28 | D |
| 1.29 | B | 1.30 | C |
| 1.31 | D | 1.32 | A |
| 1.33 | C | 1.34 | D |
| 1.35 | C | 1.36 | A |
| 1.37 | C | 1.38 | B |
| 1.39 | A | 1.40 | A |
| 1.41 | C | 1.42 | C |
| 1.43 | A | 1.44 | D |
| 1.45 | D | 1.46 | D |
| 1.47 | A | 1.48 | A |
| 1.49 | D | 1.50 | A |
| 1.51 | D | 1.52 | D |
| 1.53 | D | 1.54 | B |
| 1.55 | C | 1.56 | D |
| 1.57 | A | 1.58 | A |
| 1.59 | D | 1.60 | D |
| 1.61 | A | 1.62 | D |
| 1.63 | D | 1.64 | D |
| 1.65 | D | 1.66 | B |
| 1.67 | A | 1.68 | A |
| 1.69 | A | 1.70 | A |
| 1.71 | A | 1.72 | A |
| 1.73 | B | 1.74 | A |
| 1.75 | A | 1.76 | A |
| 1.77 | C | 1.78 | A |
| 1.79 | A | 1.80 | B |
| 1.81 | A | 1.82 | C |
| 1.83 | A | 1.84 | A |
| 1.85 | B | 1.86 | A7 |
| 1.87 | A | 1.88 | A |
| 1.89 | C | 1.90 | A |

wherein A = IC₅₀ ≤ 0.5; B = 0.5 < IC₅₀ ≤ 2.0; C = 2.0 < IC₅₀ ≤ 5.0; and D = IC₅₀ > 5.0 (unit: μM)

### Example-B2: Cell Viability Assay in Liver, Lung, Pancreatic and Glioblastoma Cell Lines

Different cell lines such as pancreatic, (MIA PaCa-2; ATCC®CRL-1420™ & and Panc-1; CRL-1469™), liver carcinoma (HepG2; ATCC® HB-8065™), lung epithelial carcinoma (A549; ATCC® CCL-185™) and others were seeded in their respective medium (DMEM, Cat #10569044, MEM, Cat #41090036; RPMI, Cat #22400089; all with 10% FBS, Gibco, ThermoFisher) at a cell count of 3000 cells per 100 μl per well in a 96 well edge plate (167425; ThermoFisher). Cells were allowed to grow at 37° C. for 24 hr in 5% CO₂ (culture conditions) in a Nuaire incubator (humidified). Serially diluted compounds (100 μl) were added to the culture plate 24 hr later and the cultures were further incubated in culture conditions for specified time depending on doubling time of specific cell line (ranging from 72-144 hr). Experiment was terminated at the completion of incubation time with the drug by replacing the medium with 100 μl of 1 mM of resazurin (R7017; Sigma) prepared in respective culture medium and the plates were further incubated in culture conditions for 4 hr. Fluorescence was then measured using a multimodal plate reader (Biotek Synergy Neo) at an excitation wavelength of 535 nm and emission wavelength of 590 nm to obtain relative fluorescence units. Data analysis was done by subtracting the background fluorescence (only medium blank) value from each reading and then normalizing with the vehicle control (DMSO treated cells) to obtain percent survival/proliferation. Percent survival at different doses was used to calculate IC₅₀ by fitting the curve to the “four-parameter variable slope logistic model” using Prism Graph Pad. IC₅₀ values are presented in Table-4 and Table-5.

TABLE 4

| | HCT116 | A549 | HepG2 | MiaPaCa-2 | Panc-1 | MDA_MB-231 | 22Rv1 |
|---|---|---|---|---|---|---|---|
| | IC50 | IC50 | IC50 | IC50 | IC50 | IC50 | IC50 |
| Comp. No. | (uM) | (uM) | (uM) | (uM) | (uM) | (uM) | (uM) |
| 1.12 | 2.64 | 2.765 | 2.635 | NA | NA | 2.75 | 1.53 |
| 1.29 | >30.0 | NA | NA | NA | NA | NA | NA |
| 1.32 | >30 | >30 | NA | NA | NA | >30 | NA |
| 1.40 | 13.44 | 28.5 | 12.56 | NA | NA | 12.63 | NA |
| 1.43 | NA | >30 | >30 | NA | >30 | >30 | >30 |
| 1.48 | >30 | >30 | >30 | >30 | NA | NA | >30 |
| 1.50 | 1.965 | >30 | >30 | >30 | NA | NA | NA |
| 1.61 | >30 | NA | >30 | NA | NA | >30 | NA |
| 1.69 | >30 | NA | 15.71 | NA | NA | 15.32 | NA |
| 1.70 | >30 | NA | 18.2 | NA | NA | >30 | NA |

### Example-B3: Mouse Splenocyte Assay (for Evaluation of Cytokine Release; mIFN-γ)

TABLE 5

| | A427 | AsPC-1 | HT29 | MV-4-11 | MCF7 | THP1 | Panc-10.05 |
|---|---|---|---|---|---|---|---|
| | IC50 | IC50 | IC50 | IC50 | IC50 | IC50 | IC50 |
| Comp. No. | (uM) | (uM) | (uM) | (uM) | (uM) | (uM) | (uM) |
| 1.12 | 3.1 | 1.82 | NA | NA | NA | 0.635 | 2.205 |
| 1.32 | >30 | >30 | NA | NA | >10 | NA | >30 |
| 1.40 | 15.24 | NA | NA | NA | NA | 6.09 | 28.47 |
| 1.43 | >30 | NA | NA | NA | >10 | NA | NA |
| 1.48 | >30 | >30 | 14.69 | 19.88 | >10 | >30 | >30 |
| 1.50 | 3.55 | NA | 3.82 | 0.70 | >10 | 13.42 | 4.72 |
| 1.61 | NA | >30 | NA | NA | >30 | NA | NA |
| 1.69 | >30 | NA | NA | 16.3 | 18.7 | 13.0 | NA |
| 1.70 | >30 | NA | NA | 29.2 | >30 | >30 | NA |

NA = Not availabe

IC₅₀ values of compounds for reversal of TGF-β suppressed and CD3e/CD28 induced mIFNγ release was determined in mouse splenocytes isolated from Balb/c mice cultured in RPMI, Cat #22400089; 10% FBS, Gibco, ThermoFisher). Mouse splenocytes (2×10⁵ cells/well) were activated with Anti-mouse CD3e (2.5 μg/ml, coated overnight at 4° C.; Cat #14-0032-82, eBioscience) and then incubated with serial dilutions of compounds (3 fold, 8 point dose response starting at 3 μM) in the presence of mouse TGF-β (10 ng/ml; Cat #14-8342-80, ebiosciences) for 30 min at 37° C., 5% CO₂ in an incubator (cell culture conditions) prior to treating them with Anti-mouse CD28 (0.1 μg/ml soluble; Cat #16-0281-82, eBiosciences). Splenocytes were further incubated under cell culture conditions for 72 hr after which the supernatant was harvested, diluted to 1:50 and ELISA was performed as per the manufacturer's protocol (mIFN-γ kit; Cat #555138 & 550534, BD Biosciences). Plates were read in Biotek plate reader by measuring absorbance at 450 nm.

The reversal of TGF-β suppressed mIFN-γ release by compounds was quantified with reference to standard by normalizing the blank (negative) subtracted test readings from average of TGF-β treated activated cells and the IC₅₀ was calculated by fitting the curve to the “four-parameter variable slope logistic model” using Prism Graph Pad.

The reversal of TGF-β suppressed mIFN-γ release by compounds was quantified by first obtaining the amount of mIFN-γ (in pg/ml) released at each concentration of the compound with respect to the mIFN-γ standard. The value of mIFN-γ released in the blank (negative control wells) was then subtracted from each test value and then normalized with the average of TGF-β treated activated cells. The IC₅₀ was calculated by fitting the curve to the “four-parameter variable slope logistic model” using Prism Graph Pad.

$$Normalized mIFN \text{-} γ release = \frac{( {[ mIFN \text{-} γ ]}_{t e s t} - {[ mIFN \text{-} γ ]}_{b l a n k} )}{( {[ mIFN \text{-} γ ]}_{TGF \text{-} β} - {[ mIFN \text{-} γ ]}_{b l a n k} )}$$

IC₅₀ values are presented in Table-6.

TABLE 6

| | Inhibition of mIFN-γ release by activated |
|---|---|
| Compound No. | Mouse Splenocyte, IC₅₀ (uM) |
| 1.48 | 0.156 |
| 1.50 | 0.107 |

### Example-B4: Mouse Splenocyte Viability Assay

IC₅₀ values of compounds for determining viability in CD3e/CD28 activated mouse splenocytes isolated from Balb/c mice cultured in RPMI, Cat #22400089; with 10% FBS, Gibco, ThermoFisher). Mouse splenocytes (2×10⁵ cells/well) were activated with Anti-mouse CD3e (2.5 μg/ml, coated overnight at 4° C.; Cat #14-0032-82, eBioscience) and then incubated with serial dilutions of compounds (3 fold, 8 point dose response starting at 30 μM) for 30 min at 37° C., 5% CO₂ in an incubator (cell culture conditions) prior to treating them with Anti-mouse CD28 (0.1 μg/ml soluble; Cat #16-0281-82, eBiosciences). Splenocytes were further incubated under cell culture conditions for 72 hr following which experiment was terminated by replacing the medium with 100 μl of 1 mM of resazurin (R7017; Sigma) prepared in respective culture medium and the plates were further incubated in culture conditions for 4 hr. Fluorescence was then measured using a multimodal plate reader (Biotek Synergy Neo) at an excitation wavelength of 535 nm and emission wavelength of 590 nm to obtain relative fluorescence units. Data analysis was done by subtracting the background fluorescence (only medium blank) value from each reading and then normalizing with the average of vehicle control (DMSO treated cells) to obtain percent survival/proliferation. Percent survival at different doses was used to calculate IC₅₀ by fitting the curve to the “four-parameter variable slope logistic model” using Prism Graph Pad.

IC₅₀ values are given in Table-7.

TABLE 7

| Compound No. | Mouse Splenocyte IC₅₀ (uM) |
|---|---|
| 1.48 | >30 |
| 1.50 | >30 |

### Example-B5: Evaluation of Viability in Human PBMC's

IC₅₀ values of compounds for determining viability in CD3e/CD28 activated human PBMC's (hPBMC-34033, Cat # CC-2702; Lonza) cultured in LGM3 medium, Cat # CC #3211; Lonza). Human PBMC's (0.25×10⁶ cells/well) were activated with Anti-human CD3e (0.5 μg/ml, coated overnight at 4° C.; Cat #16-0037-81, eBioscience) and then incubated with serial dilutions of compounds (3 fold, 8 point dose response starting at 30 μM) for 30 min at 37° C., 5% CO₂ in an incubator (cell culture conditions) prior to treating them with Anti-human CD28 (0.25 μg/ml soluble; Cat #16-0289-81, eBiosciences). Human PBMC's were further incubated under cell culture conditions for 72 hr following which experiment was terminated by replacing the medium with 100 μl of 1 mM of resazurin (R7017; Sigma) prepared in culture medium and the plates were further incubated in culture conditions for 4 hr. Fluorescence was then measured using a multimodal plate reader (Biotek Synergy Neo) at an excitation wavelength of 535 nm and emission wavelength of 590 nm to obtain relative fluorescence units. Data analysis was done by subtracting the background fluorescence (only medium blank) value from each reading and then normalizing with the vehicle control (DMSO treated cells) to obtain percent survival/proliferation. Percent survival at different doses was used to calculate IC₅₀ by fitting the curve to the “four-parameter variable slope logistic model” using Prism Graph Pad.

IC₅₀ values are given in Table-8.

TABLE 8

| Compound No. | Viability in human PBMC's IC₅₀ (uM) |
|---|---|
| 1.10 | 24.46 |
| 1.12 | 18.2 |
| 1.43 | >30 |

### Example-B6: Determination of Inhibition of Phosphorylation of Smad-2 in THP-1 Cells (IC₅₀ Determination)

IC₅₀ values of compounds for determining inhibition of phosphorylation of Smad-2 in THP-1 cells (ATCC, Cat # ATCC® TIB-202™) cultured in RPMI medium, (Cat #22400089; with 10% FBS, Gibco, ThermoFisher). THP-1 cells (1.0×10⁶ cells/well; 6 well plate) were incubated with serial dilutions of compounds (3 fold, 5 point dose response starting at 10 μM) for 60 min at 37° C., 5% CO₂ in an incubator (cell culture conditions) prior to treating them with Recombinant Human TGF-β (20 ng/ml soluble; Cat #100-21, Peprotech) for 90 min. The cells were then pelleted and processed for protein estimation and evaluation of pSmad using ELISA kit from Cell Signaling (PathScan(R) Phospho-Smad2 (Ser465/467) Sandwich ELISA; Cat #7348C) as per manufacturer's protocol. Data analysis was done by subtracting the background absorbance (only negative control) value from each reading and then normalizing with the vehicle control (DMSO treated cells) to obtain percent levels of pSmad-2. Percent inhibition thus obtained (by subtracting expression levels of pSmad from 100) at different doses was used to calculate IC₅₀ by fitting the curve to the “four-parameter variable slope logistic model” using Prism Graph Pad.

IC₅₀ values are given in Table-9 and % inhibition are given in the Table-10.

TABLE 9

| Compound No. | pSmad2 IC₅₀ (uM) |
|---|---|
| 1.48 | 0.123 |
| 1.50 | 0.539 |
| 1.61 | 1.331 |

TABLE 10

% Inhibition of pSmad2

| Compound No. | 10 μg | 3 μg |
|---|---|---|
| 1.5 | 53.3 | 47.3 |
| 1.10 | 45.1 | 36.7 |
| 1.12 | 48.4 | 51.6 |
| 1.32 | 50.5 | 51.3 |

It is understood that the foregoing examples and embodiments described above are for illustrative purposes only and that various modifications or changes in light thereof will be suggested to persons skilled in the art and are to be included within the spirit and purview of this application and the scope of the appended claims.

1. A compound of Formula (I): 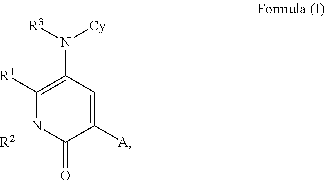
or a tautomer, salt, polymorph, solvate or stereoisomer thereof, wherein:
Cy is 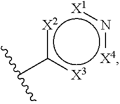
wherein
X¹ is N, CH or CR⁴;
X² is N, CH or CR⁵;
X³ is N, CH or CR⁶;
X⁴ is N, CH or CR⁷; wherein 0, 1 or 2 of X¹, X², X³ and X⁴ are N;
A is C₆ aryl, 5- to 6-membered heteroaryl, C₃-C₆ cycloalkyl or 5- to 6-membered heterocyclyl, wherein A is optionally substituted with 0 to 3 R⁹;
R¹ is independently hydrogen, halogen, —CN, C₁-C₆ alkyl, C₂-C₆ alkenyl, C₂-C₆ alkynyl, C₃-C₆ cycloalkyl, 3- to 6-membered heterocyclyl, 5- to 6-membered heteroaryl, C₁-C₃ alkoxy, C₁-C₃ haloalkoxy, C₁-C₃ haloalkyl, —OR¹¹, —NR¹¹R¹², —C(O)NR¹²R¹³, —C(O)R¹¹, —OC(O)R¹¹, —C(O)OR¹¹, —OC(O)NR¹²R¹³, —NR¹¹C(O)R¹², —NR¹¹C(O)OR¹², —NR¹¹C(O)NR¹²R¹³, —SR¹¹, —S(O)R¹¹, —S(O)₂R¹¹, —NR¹¹S(O)R¹², —C(O)NR¹¹S(O)R¹², —NR¹¹S(O)₂R¹², —C(O)NR¹¹S(O)₂R¹², —S(O)NR¹²R¹³, —S(O)₂NR¹²R¹³, —(C₁-C₃ alkylene)CF₃, —(C₁-C₃ alkylene)OR¹¹, (C₁-C₃ alkylene) cycloalkyl, —(C₁-C₃ alkylene)NR¹²R¹³, —(C₁-C₃ alkylene)C(O)R¹¹, —(C₁-C₃ alkylene)C(O)OR¹¹, —(C₁-C₃ alkylene)C(O)NR¹²R¹³, —(C₁-C₃ alkylene) —C(O)OR¹¹, —(C₁-C₃ alkylene)OC(O)NR¹²R¹³, —(C₁—C₃ alkylene)S(O)R¹¹ or —(C₁-C₃ alkylene)S(O)₂R¹¹, wherein each R¹ is optionally substituted with R¹⁰;
R² is hydrogen, C₁-C₆ alkyl, C₂-C₆ alkenyl, C₂-C₆ alkynyl, C₃-C₆ cycloalkyl, 3- to 6-membered heterocyclyl, C₁-C₃ alkoxy, C₁-C₃ haloalkoxy, —C(O) R¹¹ or —(C₁-C₃ alkylene)OR¹¹;
or R¹ and R² are taken together with the atom to which they are attached to form a 5- to 7-membered heterocyclyl containing 1 to 3 heteroatom independently selected from N, O or S; each of which is optionally substituted by 0 to 3 R⁸;
R³ is hydrogen or C₁-C₆ alkyl;
R⁴, R⁵, R⁶ and R⁷ are independently hydrogen, halogen, oxo, —CN, C₁-C₆ alkyl, C₁-C₆ cycloalkyl, C₁-C₃ alkoxy, C₁-C₃ haloalkoxy, C₁-C₃ haloalkyl, 3- to 6-membered heterocyclyl, 5-to 6-membered heteroaryl, —NR¹¹R¹², —S(O)₂NR²R¹³, —NR¹¹S(O)₂R¹², —C(O)NR¹²R¹³, —NR¹¹C(O)R¹² or —C(O)OR¹, each of which is optionally substituted by halogen, oxo, —CN, —OR¹⁵ or —NR¹⁵R¹⁶;
or R⁴ and R⁵ are taken together with the atom to which they are attached to form a 5- to 6-membered cycloalkyl or C₆ aryl; or 5- to 6-membered heterocyclyl or heteroaryl containing 1 to 3 heteroatom independently selected from N, O or S; each of which is optionally substituted by 0 to 3 R⁸;
each R⁸ is independently hydrogen, halogen, oxo, —CN, C₁-C₆ alkyl, C₁-C₆ cycloalkyl, C₁-C₃ alkoxy, C₁-C₃ haloalkoxy, C₁-C₃ haloalkyl, 3- to 6-membered heterocyclyl, -NR¹¹R¹², —C(O)NR¹²R¹³, —NR¹¹C(O)R¹², —C(O)OR¹¹, —OR¹¹, —(C₁-C₃ alkylene)NR¹¹R¹² or —(C₁-C₃ alkylene)3- to 6-membered heterocyclyl, each of which is optionally substituted by C₁-C₆ alkyl, halogen, oxo, —CN, —OR¹⁵ or NR¹⁵R¹⁶;
each R⁹ is independently halogen, —CN, C₁-C₆ alkyl, C₃-C₆ cycloalkyl, 3- to 6-membered heterocyclyl, C₁-C₃ haloalkoxy, C₁-C₃ haloalkyl, —OR¹¹, —NR¹¹ R¹², —C(O) NR¹² R¹³, —C(O) R¹¹, —SR¹¹, —S(O)R¹² or —S(O)₂R¹¹, each of which is optionally substituted by halogen, oxo, —CN, —OR¹⁵ or —NR¹⁵R¹⁶;
R¹⁰ is independently hydrogen, halogen, oxo, —CN, C₁-C₆ alkyl, C₃-C₆ cycloalkyl, 3- to 6-membered heterocyclyl, C₁-C₃ haloalkoxy, C₁-C₃ haloalkyl, —OR¹⁵, —NR¹⁵ R¹⁶, —C(O) NR¹⁵ R¹⁶, —C(O) R¹⁵, —SR¹⁵, —S(O)R¹⁵ or —S(O)₂R¹⁵;
R¹¹, R¹² and R¹³ are independently hydrogen, C₁-C₆ alkyl, C₂-C₆ alkenyl, C₂-C₆ alkynyl, C₃-C₆ cycloalkyl, 3- to 6-membered heterocyclyl, 5- to 6-membered heteroaryl or —(C₁-C₃ alkylene)OR¹⁵, each of which is optionally substituted with C₃-C₆ cycloalkyl, halogen, oxo, C₁-C₃ alkoxy, 3- to 6-membered heterocyclyl, —OR¹⁵, —NR¹⁵R¹⁶, —C(O)NR¹⁵R¹⁶, —NR¹⁵C(O)R¹⁶, C(O)R¹⁵, —S(O)₂R¹⁵ —C(O)NR¹⁵S(O)₂R¹⁶ or C₁-C₆ alkyl optionally substituted by halogen, oxo, CN, —NH₂ or —OH;
or R¹¹ and R¹² are taken together with the atom to which they attached to form a 3- to 6-membered heterocyclyl optionally substituted by C₁-C₆ alkyl, halogen, oxo, —CN, —OR¹⁵ or —NR¹⁵R¹⁶;
or R¹² and R¹³ are taken together with the atom to which they attached to form a 3- to 6-membered heterocyclyl optionally substituted by C₁-C₆ alkyl, halogen, oxo, —CN, —NH₂ or —OH;
R¹⁵ and R¹⁶ are each independently hydrogen, C₁-C₆ alkyl, C₂-C₆ alkenyl, or C₂-C₆ alkynyl, wherein each is optionally substituted by C₁-C₆ alkyl, halogen, oxo, —CN, —NH₂ or —OH; or
or R¹⁵ and R¹⁶ are taken together with the atom to which they attached to form a 3- to 6-membered heterocyclyl optionally substituted by halogen, oxo, —CN, —NH₂ or —OH;
provided that:
i) when R¹ is hydrogen and R⁴ and R⁵ are taken together with the atom to which they attached to form a 5-membered heteroaryl then R⁸ is other than 6-membered heterocyclyl; or
ii) when R³ is —CH₃ then R⁴ or R⁷ is not —NR¹¹R¹².

2. The compound of claim 1, wherein Cy 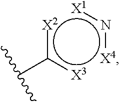
wherein:
X¹ is N, CH or CR⁴;
X² is N, CH or CR⁵;
X³ is N, CH or CR⁶;
X⁴ is N, CH or CR⁷; wherein 0, 1 or 2 of X¹, X², X³ and X⁴ are N;
R⁴, R⁵, R⁶ and R⁷ are independently hydrogen, halogen, oxo, —CN, C₁-C₆ alkyl, C₁-C₆ cycloalkyl, C₁-C₃ alkoxy, C₁-C₃ haloalkoxy, C₁-C₃ haloalkyl, 3- to 6-membered heterocyclyl, 5- to 6-membered heteroaryl, —NR¹¹R¹², —S(O)₂NR¹²R¹³, —NR¹¹S(O)₂R¹², —C(O)NR¹²R¹³, —NR¹¹C(O)R¹² or —C(O)OR¹¹, each of which is optionally substituted by halogen, oxo, —CN, —OR¹⁵ or —NR¹⁵R¹⁶;
R¹¹, R¹², R¹³, R¹⁵ and R¹⁶ are as defined in claim 1;
provided that when R³ is —CH₃ then R⁴ or R⁷ is not —NR¹¹R¹².

3. The compound of claim 1, wherein Cy 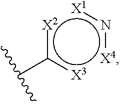
wherein:
X¹ is CR⁴;
X² is CR⁵;
X³ is N, CH or CR⁶;
X⁴ is N, CH or CR⁷; wherein 0, 1 or 2 of X¹, X², X³ and X⁴ are N;
R⁴ and R⁵ are taken together with the atom to which they attached to form a 5-membered cycloalkyl; or 5-membered heterocyclyl or heteroaryl containing 1 to 3 heteroatom independently selected from N, O or S; each of which is optionally substituted by 0 to 3 R⁸;
R⁶, R⁷ and R⁸ are as defined in claim 1;
provided that when R¹ is hydrogen and R⁴ and R⁵ are taken together with the atom to which they attached to form a 5-membered heteroaryl then R⁸ is other than 6-membered heterocyclyl.

4. The compound of claim 1, wherein Cy 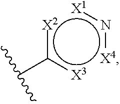
wherein:
X¹ is CR⁴;
X² is CR⁵;
X³ is N, CH or CR⁶;
X⁴ is N, CH or CR⁷; wherein 0, 1 or 2 of X¹, X², X³ and X⁴ are N;
R⁴ and R⁵ are taken together with the atom to which they attached to form a 6-membered cycloalkyl or C₆ aryl; or 6-membered heterocyclyl or heteroaryl containing 1 to 3 heteroatom independently selected from N, O or S; each of which is optionally substituted by 0 to 3 R⁸;
R⁶, R⁷ and R⁸ are as defined in claim 1.

5. The compound of claim 2, wherein Cy is selected from the group consisting

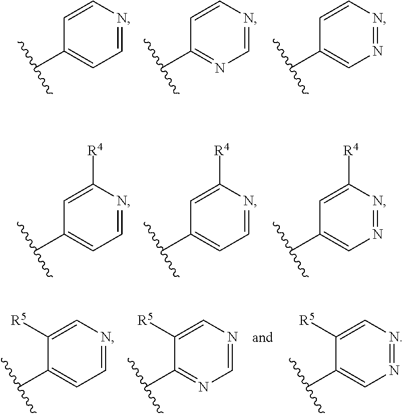

6. The compound of claim 5, wherein R⁴ is selected from the group consisting of —NH₂,

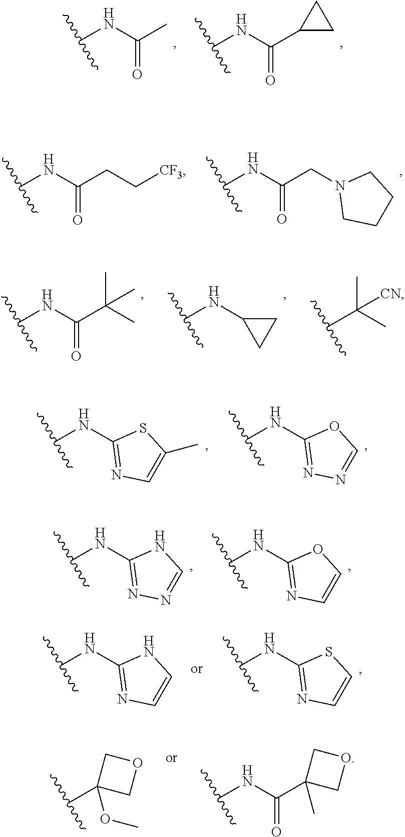

7. The compound of claim 5, wherein R⁴ is

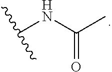 [SMILES: *NC(C)=O]

8. The compound of claim 5, wherein R⁵ is selected from the group consisting of, —CN, F,

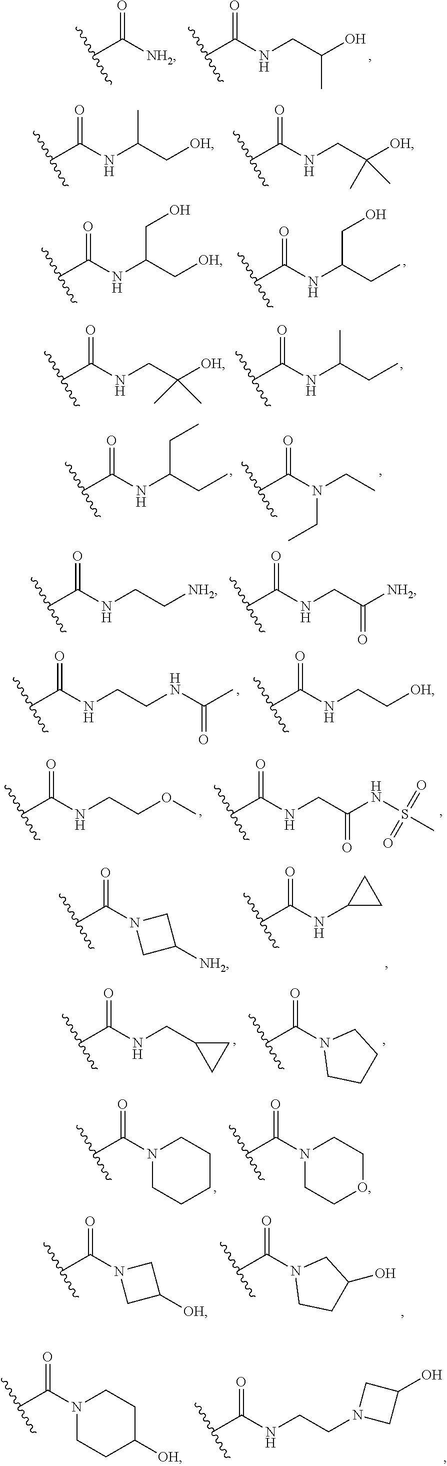

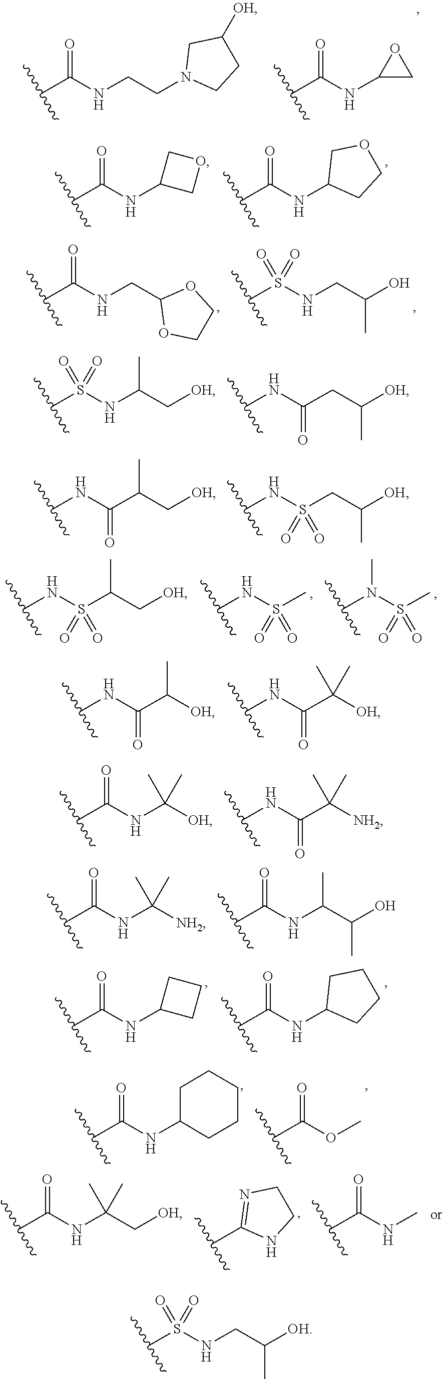

9. The compound of claim 5, wherein R⁵ is selected from the group consisting of

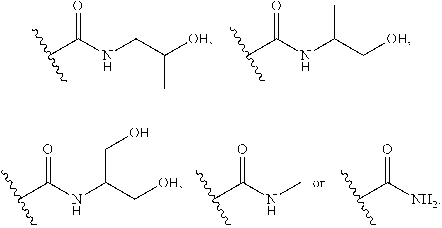

10. The compound of claim 3, wherein Cy is selected from the group consisting of

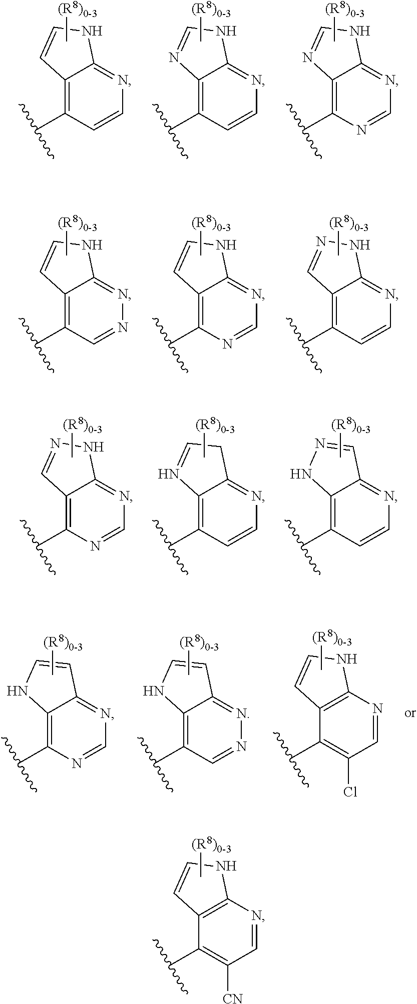

11. The compound of claim 3, wherein Cy is selected from the group consisting of

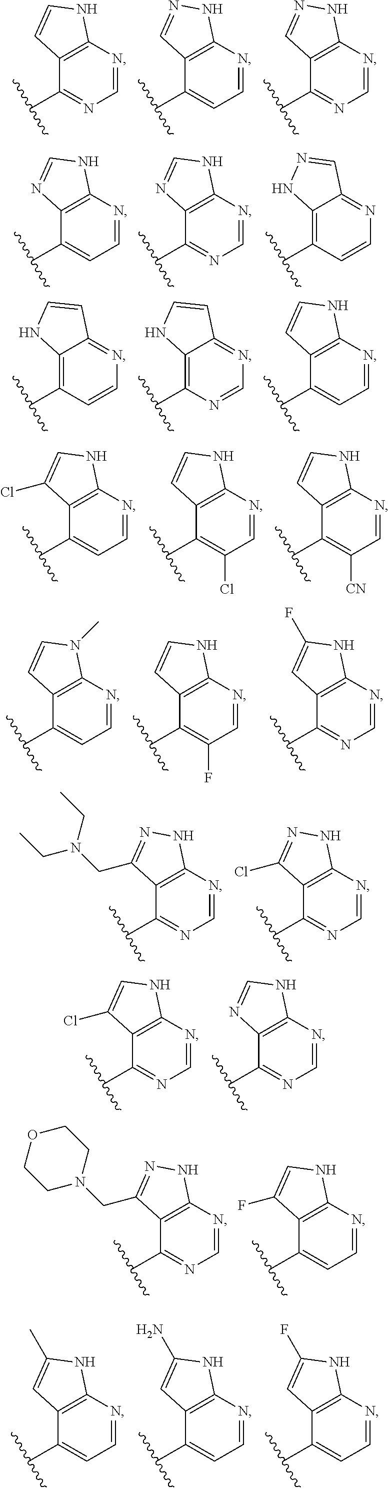

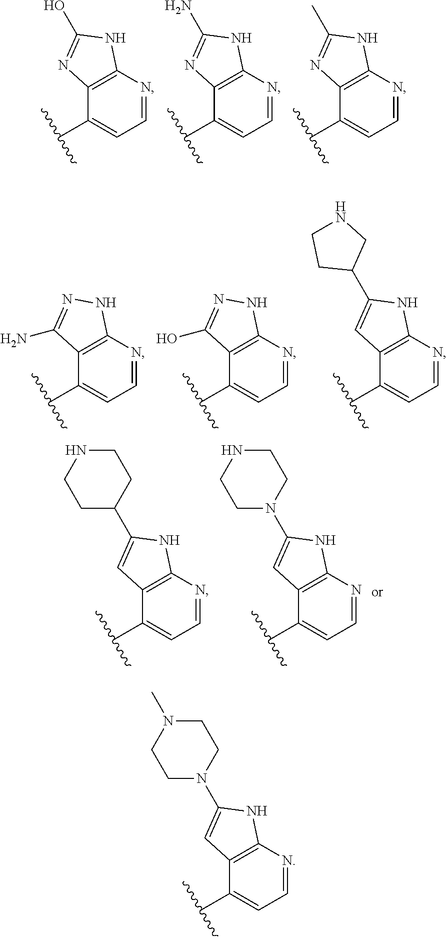

12. The compound of claim 4, wherein Cy is selected from the group consisting of

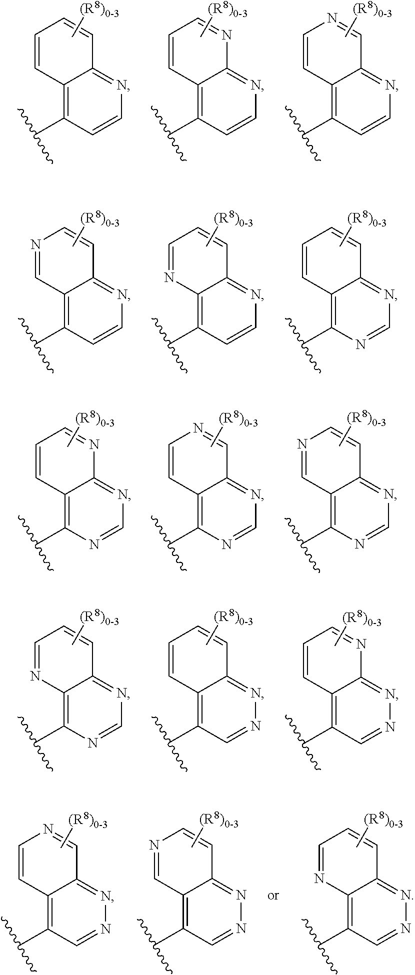

13. The compound of claim 4, wherein Cy is selected from the group consisting of

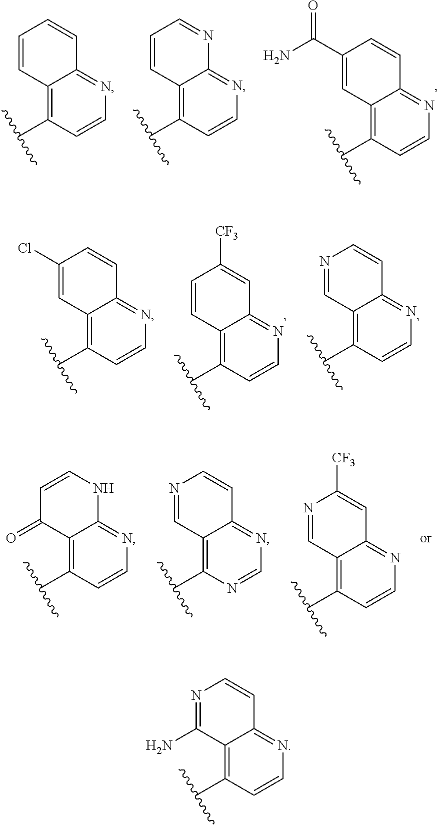

14. The compound of claim 1, wherein Cy is selected from the group consisting of

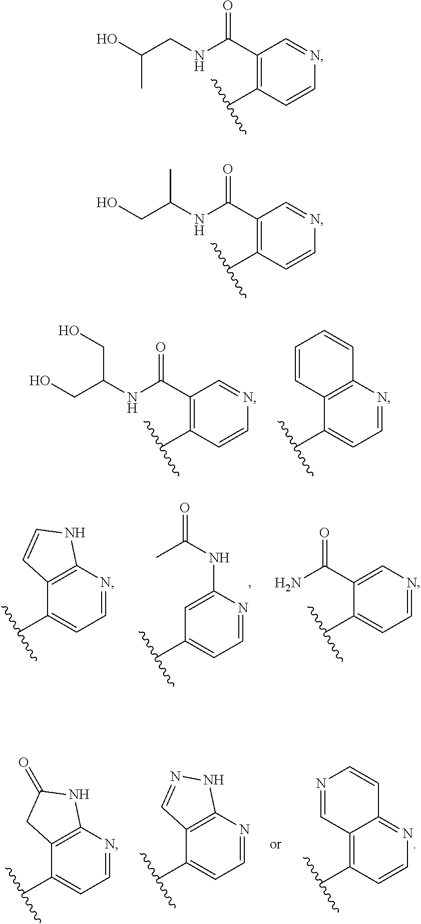

15. The compound of claim 1, wherein A is C₆ aryl or phenyl optionally substituted with 0 to 3 R⁹.

16. The compound of claim 1, wherein A is 5- to 6-membered heteroaryl optionally substituted with 0 to 3 R⁹.

17. The compound of claim 15, wherein A is selected from the groups consisting of

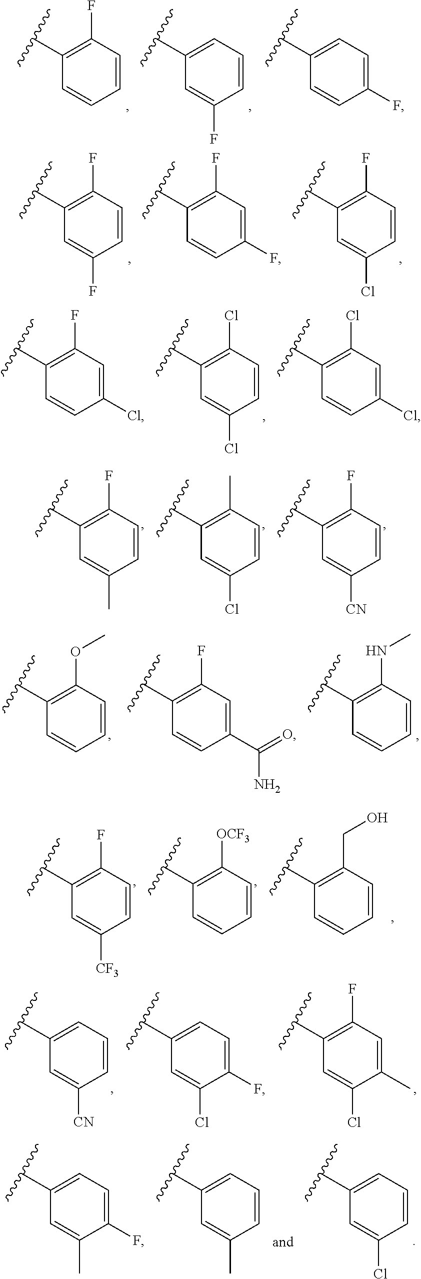

18. The compound of claim 15, wherein A is selected from the groups consisting of

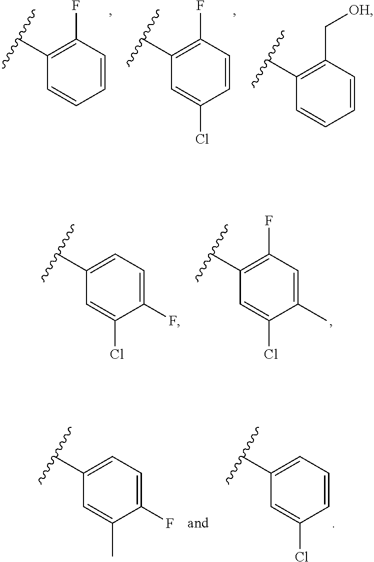

19. The compound of claim 16, wherein A is selected from the groups consisting of,

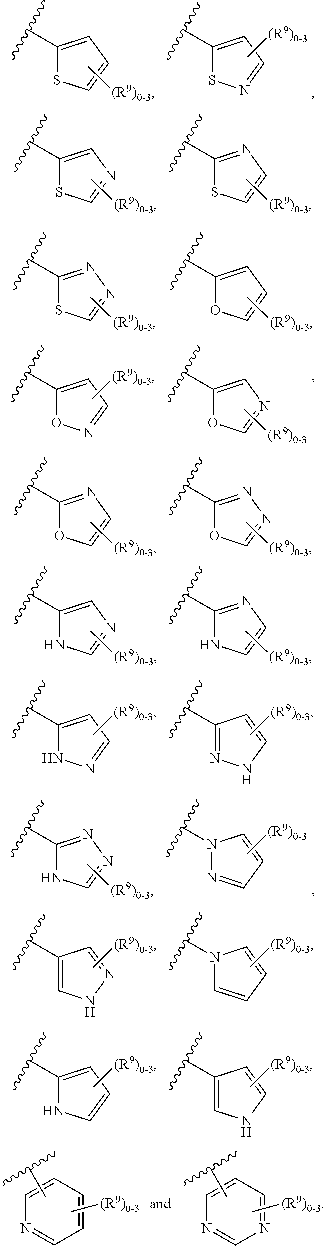

20. The compound of claim 16, wherein A is selected from the groups consisting of

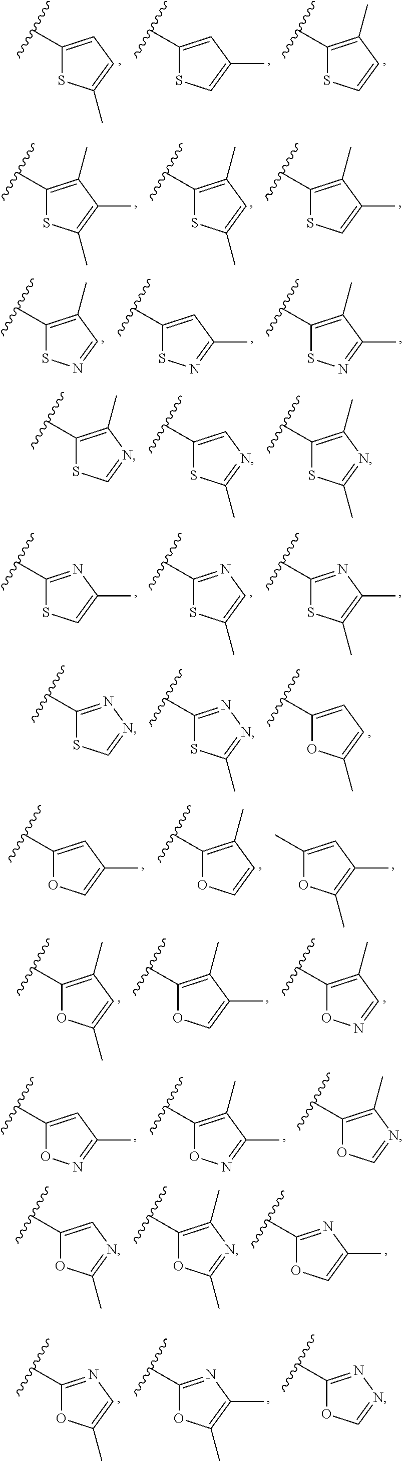

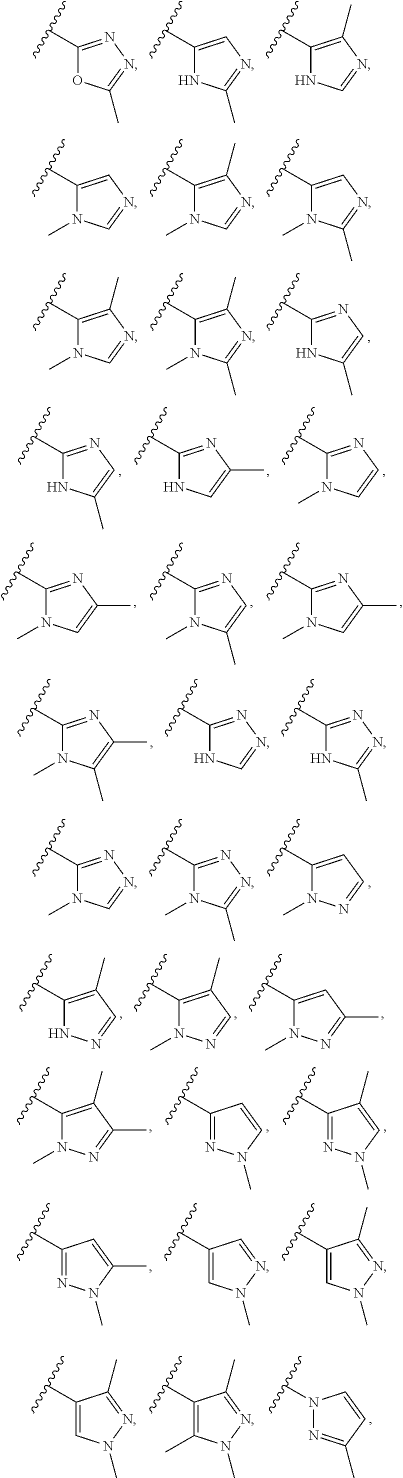

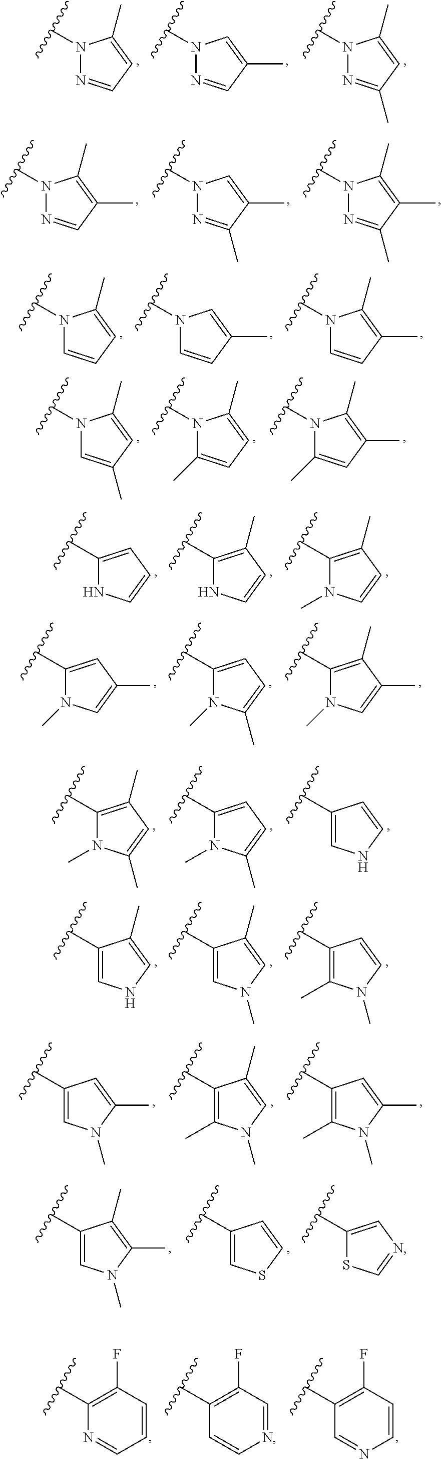

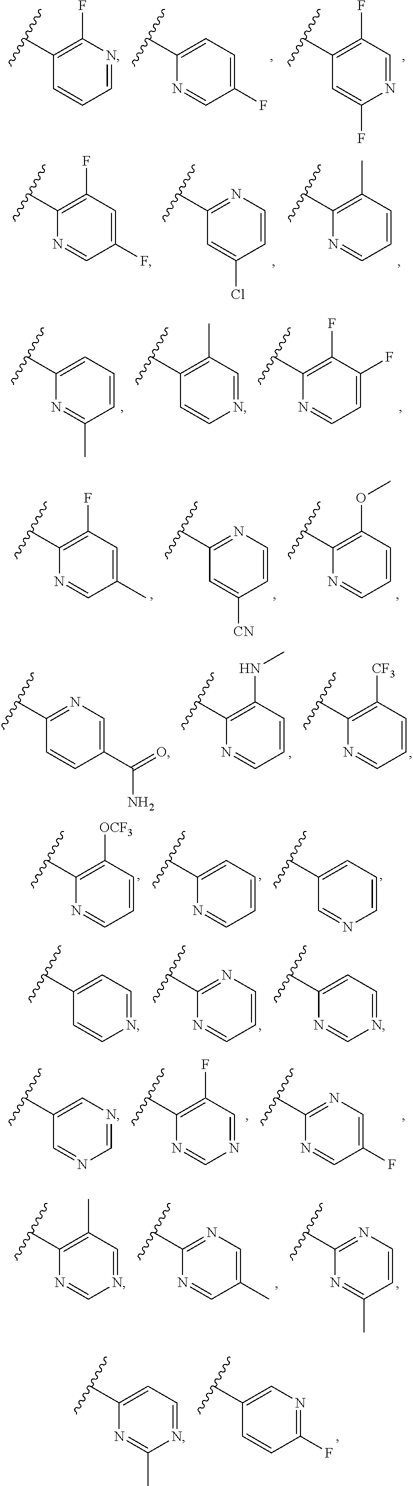

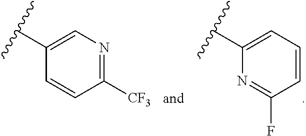 [SMILES: *c1ccc(C(F)(F)F)nc1 ; *c1cccc(F)n1]

21. The compound of claim 16, wherein A is

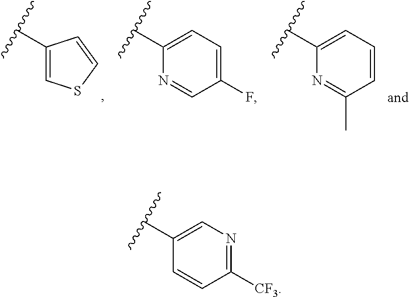

22. The compound of claim 1, wherein R¹ is selected from hydrogen, —CN, C₁-C₆ alkyl, 3- to 6-membered heterocyclyl, 5- to 6-membered heteroaryl, C₁-C₃ alkoxy, C₁-C₃ haloalkoxy, C₁-C₃ haloalkyl, —OR¹¹, —NR¹¹ R¹², —C(O) NR¹² R¹³ or —S(O)₂R¹¹.

23. The compound of claim 22, wherein R¹ is selected from hydrogen, ethyl, isopropyl, cyclopropyl, dimethyl amine, —N(CH₃)₂, —S—CH₃ and —O—CH₃.

24. The compound of claim 22, wherein R¹ is hydrogen.

25. The compound of claim 22, wherein R¹ is ethyl.

26. The compound of claim 22, wherein R¹ is isopropyl.

27. The compound of claim 22, wherein R¹ is cyclopropyl.

28. The compound of claim 22, wherein R¹ is —N(CH₃)₂.

29. The compound of claim 22, wherein R¹ is —S—CH₃.

30. The compound of claim 22, wherein R¹ is —O—CH₃.

31. The compound of claim 1, wherein R² is selected from the groups consisting of hydrogen, C₁-C₆ alkyl.

32. The compound of claim 31, wherein R² is hydrogen.

33. The compound of claim 31, wherein R² is methyl.

34. The compound of claim 1, wherein R¹ and R² are taken together with the atom to which they attached to form a 5- to 7-membered heterocyclyl containing 1 to 3 heteroatom independently selected from N, O or S; each of which is optionally substituted by 0 to 3 R⁸.

35. The compound of claim 34, wherein the R¹ and R² are taken together to form

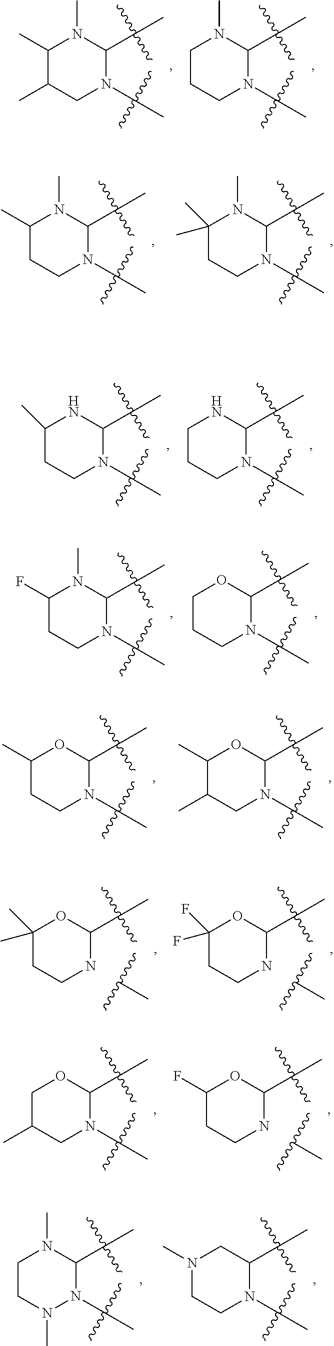

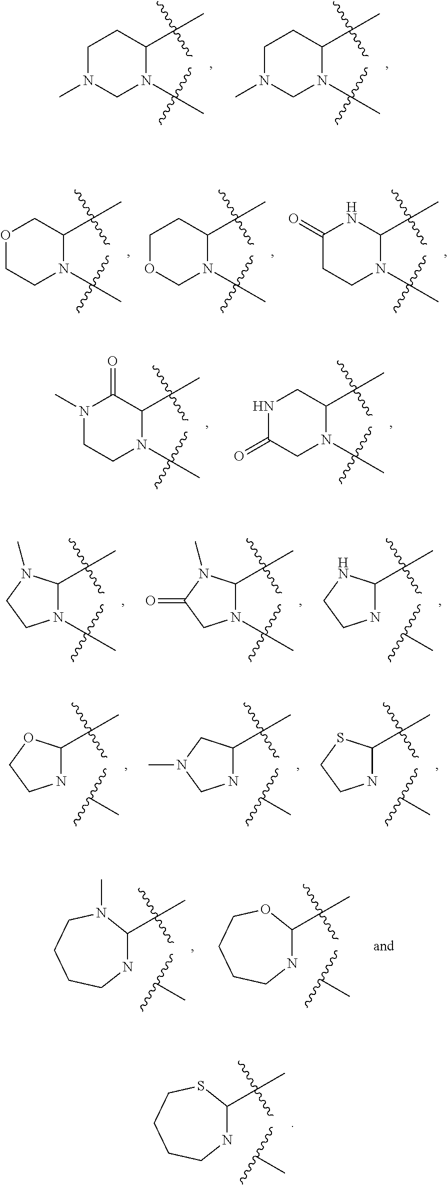

36. The compound of claim 34, wherein the R¹ and R² are taken together to form

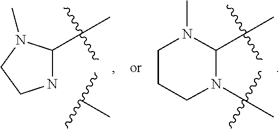

37. The compound of claim 1, wherein R³ is H.

38. The compound of claim 1, wherein R³ is CH₃.

39. The compound of claim 1, wherein the compound is a compound of formula (II): 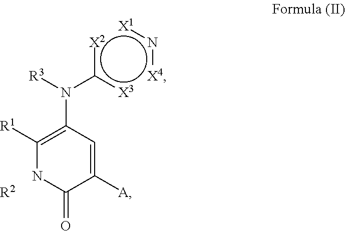
or a tautomer, salt, polymorph, solvate or stereoisomer thereof, wherein:
X¹ is N, CH or CR⁴;
X² is N, CH or CR⁵;
X³ is N, CH or CR⁶;
X⁴ is N, CH or CR⁷; wherein 0, 1 or 2 of X¹, X², X³ and X⁴ are N;
R⁴, R⁵, R⁶ and R⁷ are independently hydrogen, halogen, oxo, —CN, C₁-C₆ alkyl, C₁-C₆ cycloalkyl, C₁-C₃ alkoxy, C₁-C₃ haloalkoxy, C₁-C₃ haloalkyl, 3- to 6-membered heterocyclyl, 5-to 6-membered heteroaryl, —NR¹¹R¹², —S(O)₂NR¹²R¹³, —NR¹¹S(O)₂R¹², —C(O)NR¹²R¹³, —NR¹¹C(O)R¹², —C(O)OR¹¹, each of which is optionally substituted by halogen, oxo, —CN, —OR¹⁵ or —NR¹⁵R¹⁶;
A, R¹, R², R³, R¹¹, R¹², R¹³, R¹⁵ and R¹⁶ are as defined in claim 1;
provided that when R³ is —CH₃ then R⁴ or R⁷ is not —NR¹¹R¹².

40. The compound of claim 1, wherein the compound is a compound of formula (III): 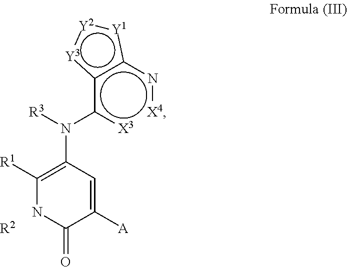
or a tautomer, salt, polymorph, solvate or stereoisomer thereof, wherein:
X³ is CH, N or CR⁶;
X⁴ is CH, N or CR⁷;
Y¹ and Y³ are independently N, NH, NR⁸, CH or CR⁸, S, O;
Y² is N, CH or CR⁸;
A, R¹, R², R³, R⁶, R⁷ and R⁸ are as defined in claim 1;
provided that when R¹ is hydrogen then R⁸ is other than 6-membered heterocyclyl.

41. The compound of claim 1, wherein the compound is a compound of formula (IV): 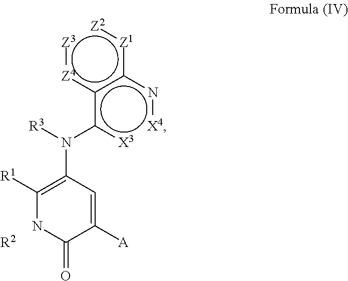
or a tautomer, salt, polymorph, solvate or stereoisomer thereof, wherein:
X³ is N, CH or CR⁶;
X⁴ is N, CH or CR⁷;
Z¹, Z², Z³ and Z⁴ are independently N, CH or CR⁸;
A, R¹, R², R³, R⁶, R⁷ and R⁸ are as defined in claim 1.

42. The compound of claim 1, wherein the compound is a compound of formula (V): 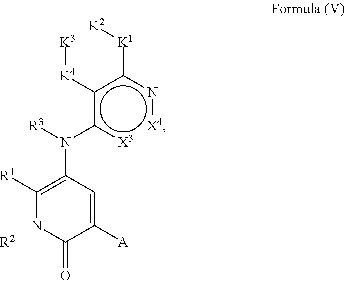
or a tautomer, salt, polymorph, solvate or stereoisomer thereof, wherein:
X³ is N, CH or CR⁶;
X⁴ is N, CH or CR⁷;
K¹ is S, O, NH, NR⁸, CH₂, CHR⁸, C═O or CR⁸R^{8′};
K² is NH, NR⁸, CH₂, CHR⁸, C═O or CR⁸R^{8′};
K³ is NH, NR⁸, CH₂, CHR⁸, C═O or CR⁸R^{8′} or absent;
K⁴ is S, O, NH, NR⁸, CH₂, CHR⁸, C═O or CR⁸R^{8′};
provided that:
i) no more than two of K¹, K², K³ and K⁴ are NH or NR⁸;
ii) if K³ is absent, then at least one of K¹ and K⁴ is not O or S;
iii) no more than two of K¹, K², K³ and K⁴ is C═O;
R⁸ and R^{8′} are independently hydrogen, halogen, oxo, —CN, C₁-C₆ alkyl, C₁-C₆ cycloalkyl, C₁-C₃ alkoxy, C₁-C₃ haloalkoxy, C₁-C₃ haloalkyl, 3- to 6-membered heterocyclyl, —NR¹¹R¹², —C(O)NR¹²R¹³, —NR¹¹C(O)R¹², —C(O)OR¹¹, —OR¹¹, —(C₁-C₃ alkylene)NR¹¹R¹² or —(C₁-C₃ alkylene)3- to 6-membered heterocyclyl, each of which is optionally substituted by C₁-C₆ alkyl, halogen, oxo, —CN, —OR¹⁵ or —NR¹⁵R¹⁶;
A, R¹, R², R³, R¹¹, R¹², R¹³, R¹⁵ and R¹⁶ are as defined in claim 1.

43. The compound of claim 39, wherein the compound is selected from any of the compound of formula II-A to II-X.

44. The compound of claim 40, wherein the compound is selected from any of the compound of formula III-A to III-P.

45. The compound of claim 41, wherein the compound is selected from any of the compound of formula IV-A to IV-P.

46. The compound of claim 42, wherein the compound is selected from any of the compound of formula V-A to V-L.

47. The compound of claim 1, wherein the compound is selected from Compound Nos. 1.1 to 1.90 in Table 1 or a tautomer, salt, polymorph, solvate or stereoisomer thereof.

48. The compound of claim 1, wherein the compound is selected from Compound Nos. 2.1 to 2.386 in Table 2 or a tautomer, salt, polymorph, solvate or stereoisomer thereof.

49. A pharmaceutical composition comprising a compound of claim 1, or a tautomer, salt, polymorph, solvate or stereoisomer thereof, and a pharmaceutically acceptable carrier.

50. A method of treating disease associated with excessive activity of transforming growth factor beta (TGF-β) in an individual in need thereof comprising administering to the individual a therapeutically effective amount of a compound of claim 1, or a pharmaceutically acceptable salt thereof.

51. The method of treating of claim 50, wherein the transforming growth factor beta (TGF-β) is type 1 transforming growth factor beta (TGF-β) or activin-like kinase 5 (ALK-5).

52. A method of treating cancer in an individual in need thereof comprising administering to the individual a therapeutically effective amount of a compound of claim 1, or a tautomer, salt, polymorph, solvate or stereoisomer thereof.

53. A method of inhibiting transforming growth factor beta (TGF-β) type 1 or activin-like kinase 5 (ALK-5) receptor comprising administering a compound of claim 1, or a tautomer, salt, polymorph, solvate or stereoisomer thereof.

55. Use of a compound of claim 1, or a pharmaceutically acceptable salt or solvate thereof, in the manufacture of a medicament for treatment of a disease mediated by a TGF-β receptor kinase; preferably type 1 or activin-like kinase 5 (ALK-5).

56. A kit comprising a compound of claim 1, or a tautomer, salt, polymorph, solvate or stereoisomer thereof.
